(19) United States
(12) Patent Application Publication
(10) Pub. No.: US 2004/0023267 A1
(43) Pub. Date: Feb. 5, 2004

(54) Novel compositions and methods in cancer

(76) Inventor: David W. Morris, Davis, CA (US)

Correspondence Address: MORRISON & FOERSTER LLP, 755 PAGE MILL RD, PALO ALTO, CA, 94304-1018, US

(21) Appl. No.: 10/394,948

(22) Filed: Mar. 21, 2003

Related U.S. Application Data
(60) Provisional application No. 60/367,025, filed on Mar. 21, 2002.

(51) Int. Cl. C12Q 1/68; C07H 21/04; C12M 1/34

(52) U.S. Cl. 435/6; 435/287.2; 536/23.2

(57) ABSTRACT

The present invention relates to novel sequences for use in detection, diagnosis and treatment of cancers. The invention provides cancer-associated (CA) polynucleotide sequences whose expression is associated with cancer. The present invention provides CA polypeptides associated with cancer and provides diagnostic compositions and methods for the detection of cancer. The present invention provides monoclonal and polyclonal antibodies specific for the CA polypeptides. The present invention also provides diagnostic tools and therapeutic compositions and methods for screening, prevention and treatment of cancer.

### CROSS-REFERENCE TO RELATED APPLICATIONS

This application claims priority to U.S. provisional application Ser. No. 60/367,025 entitled “Novel Compositions and Methods in Cancer,” filed Mar. 21, 2002. This application is related to U.S. Applications entitled “Novel Compositions and Methods in Cancer,” U.S. Ser. No. 09/747,377, filed Dec. 22, 2000, U.S. Ser. No. 09/798,586, filed Mar. 2, 2001, U.S. Ser. No. 10/004,113, filed Oct. 23, 2001, U.S. Ser. No. 10/052,482, filed Nov. 8, 2001 and U.S. Ser. No. 09/997,722, filed Nov. 30, 2001, all of which are expressly incorporated herein by reference in their entirety.

### TECHNICAL FIELD OF THE INVENTION

This invention relates generally to the field of cancer-associated genes. Specifically, it relates to novel sequences for use in diagnosis and treatment of cancer and tumors, as well as the use of the novel compositions in screening methods. The present invention provides methods of using cancer associated polynucleotides, their corresponding gene products and antibodies specific for the gene products in the detection, diagnosis, prevention and/or treatment of associated cancers.

### BACKGROUND OF THE INVENTION

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes. Carcinogenesis is fundamentally driven by somatic cell evolution (i.e. mutation and natural selection of variants with progressive loss of growth control). The genes that serve as targets for these somatic mutations are classified as either protooncogenes or tumor suppressor genes, depending on whether their mutant phenotypes are dominant or recessive, respectively.

There are a number of viruses known to be involved in human cancer as well as in animal cancer. Of particular interest here are viruses that do not contain oncogenes themselves; these are slow-transforming retroviruses. They induce tumors by integrating into the host genome and affecting neighboring protooncogenes in a variety of ways. Provirus insertion mutation is a normal consequence of the retroviral life cycle. In infected cells, a DNA copy of the retrovirus genome (called a provirus) is integrated into the host genome. A newly integrated provirus can affect gene expression in cis at or near the integration site by one of two mechanisms. Type I insertion mutations up-regulate transcription of proximal genes as a consequence of regulatory sequences (enhancers and/or promoters) within the proviral long terminal repeats (LTRs). Type II insertion mutations cause truncation of coding regions due to either integration directly within an open reading frame or integration within an intron flanked on both sides by coding sequences. The analysis of sequences at or near the insertion sites has led to the identification of a number of new protooncogenes.

With respect to lymphoma and leukemia, retroviruses such as AKV murine leukemia virus (MLV) or SL3-3 MLV, are potent inducers of tumors when inoculated into susceptible newborn mice, or when carried in the germline. A number of sequences have been identified as relevant in the induction of lymphoma and leukemia by analyzing the insertion sites; see Sorensen et al., J. of Virology 74:2161 (2000); Hansen et al., Genome Res. 10(2):237-43 (2000); Sorensen et al., J. Virology 70:4063 (1996); Sorensen et al., J. Virology 67:7118 (1993); Joosten et al., Virology 268:308 (2000); and Li et al., Nature Genetics 23:348 (1999); all of which are expressly incorporated by reference herein. With respect to cancers, especially breast cancer, prostate cancer and cancers with epithelial origin, the mammalian retrovirus, mouse mammary tumor virus (MMTV) is a potent inducer of tumors when inoculated into susceptible newborn mice, or when carried in the germ line. Mammary Tumors in the Mouse, edited by J. Hilgers and M. Sluyser; Elsevier/North-Holland Biomedical Press; New York, N.Y.

Breast cancer is one of the most significant diseases that affects women. At the current rate, American women have a 1 in 8 risk of developing breast cancer by age 95 (American Cancer Society, 1992). Treatment of breast cancer at later stages is often futile and disfiguring, making early detection a high priority in medical management of the disease.

The pattern of gene expression in a particular living cell is characteristic of its current state. Nearly all differences in the state or type of a cell are reflected in the differences in RNA levels of one or more genes. Comparing expression patterns of uncharacterized genes may provide clues to their function. High throughput analysis of expression of hundreds or thousands of genes can help in (a) identification of complex genetic diseases, (b) analysis of differential gene expression over time, between tissues and disease states, and (c) drug discovery and toxicology studies. Increase or decrease in the levels of expression of certain genes correlate with cancer biology. For example, oncogenes are positive regulators of tumorigenesis, while tumor suppressor genes are negative regulators of tumorigenesis. (Marshall, Cell, 64: 313-326 (1991); Weinberg, Science, 254: 1138-1146 (1991)).

SNL, which also in known as fascin, is an actin-bundling protein that was first isolated from cytoplasmic extracts of sea urchin eggs (Kane, 1975: J. Cell Biol. 66:305-315) and was the first bundling protein to be characterized in vitro. Subsequent work has shown that fascin bundles actin filaments in fertilized egg microvilli and filopodia of phagocytic coelomocytes (Otto et al., 1980, Cell Motil. 1:31-40; Otto and Bryan, 1981, Cell Motil. 1: 179-192). Fascin is a widely expressed protein found in a broad spectrum of tissues and organisms.

In vivo, AP-1, or transcription factor activating protein 1, is a heterogenous mixture of heterodimers of several related protein subunits in addition to c-Fos and c-Jun including FosB, Fra-1, Fra-2, c-Jun, JunB, JunD, etc. (The FOS and JUN Families of Proteins, Angel and Herrlich, eds., CRC Press, Boca Raton, Fla., 1994). While AP-1 has been implicated in abnormal cell proliferation and tumor formation, events that thus might be controlled by modulating the expression of c-fos and/or c-jun, the precise contribution of each of these proteins to cell proliferation or tumor formation is unclear.

The c-myc protein is a member of the helix-loop-helix/leucine zipper (HLH/LZ) family of transcription factors that forms heterodimers with Max. In general, trans-activating Myc:Max heterodimers are found in proliferating cells, while trans-repressing Mad:Max heterodimers are found in differentiated cells. The c-myc protein level influences cell proliferation, differentiation, and neoplastic transformation, presumably by affecting the balance between Myc:Max and Mad:Max heterodimers.

Cyclin D1 is a protein derived from the PRAD1, CCND1 or bcl-1 gene on chromosome 11q13, which is involved in both regulation of the cell cycle. In the G1 (resting) phase of the cell cycle, cyclin D1 together with its cyclin dependent kinase (cdk) partner, is responsible for transition to the S (DNA synthesis) phase by phosphorylating the product of the retinoblastoma gene (pRB), which then releases transcription factors important in the initiation of DNA replication. The nuclear factor-κ B is an inducible transcription factor which participates in the regulation of multiple cellular genes after treatment of cells with factors such as phorbol ester, lipopolysaccharide (LPS), interleukin-1 (IL-1) and tumor necrosis factor-α (TNF-alpha). These genes are involved in the immediate early processes of immune, acute phase, and inflammatory responses. NF-κB has also been implicated in the transcriptional activation of several viruses, most notably the type 1 human immunodeficiency virus (HIV-1) and cytomegalovirus (CMV) (Nabel, et al., Nature, 326:711, 1987; Kaufman, et al., Mol. Cell. Biol., 7:3759, 1987; Sambucetti, et al., EMBO J, 8:4251, 1989). NF-κB is a dimeric transcription factor that binds and regulates gene expression through decameric cis-acting NF-κB DNA motifs. Although a p50/p65 heterodimer has traditionally been referred to as NF-κB and remains the prototypical and most abundant form, it has been recognized recently that several distinct but closely related homo- and heterodimeric factors are responsible for NF-κB site-dependent DNA binding activity and regulation. The various dimeric factors are composed of members of the family of Rel-related polypeptides. One subclass of this family, distinguished by its proteolytic processing from precursor forms and lack of recognized activation domains, includes p50 (NFKB1) and p50B (NFKB2, p52), whereas the second subclass contains recognized activation domains and includes p65 (RelA), RelB, c-Rel, and the Drosophila protein Dorsal. All Rel-related members share a 300-amino acid region of homology, RHD, responsible for DNA binding and dimerization, called the Rel homology domain. In the cytoplasm, NF-κB and Rel proteins form a “Rel complex”.

NF-κB gene regulation is involved in many pathological events including progression of acquired immune deficiency disease (AIDS), the acute phase response and the activation of immune and endothelial cells during toxic shock, allograft rejection, and radiation responses. In addition, NF-κB gene transactivation may be critical for HIV and CMV replication.

The PVT-1 locus is associated with the c-myc locus. Frequently, mutations in one or the other loci correlate with disease such as lymphoma. While rearrangements and amplification of the PVT locus have been found in lymphomas, the role of PVT-1 remains unclear.

Accordingly, it is an object of the invention to provide polynucleotide and polypeptide sequences involved in cancer and, in particular, in oncogenesis.

Immunotherapy, or the use of antibodies for therapeutic purposes has been used in recent years to treat cancer. Passive immunotherapy involves the use of monoclonal antibodies in cancer treatments. See for example, Cancer: Principles and Practice of Oncology, 6^{th} Edition (2001) Ch. 20, pp. 495-508. Inherent therapeutic biological activity of these antibodies include direct inhibition of tumor cell growth or survival, and the ability to recruit the natural cell killing activity of the body's immune system. These agents are administered alone or in conjunction with radiation or chemotherapeutic agents. Rituxan® and Herceptin®, approved for treatment of lymphoma and breast cancer, respectively, are two examples of such therapeutics. Alternatively, antibodies are used to make antibody conjugates where the antibody is linked to a toxic agent and directs that agent to the tumor by specifically binding to the tumor. Mylotarg® is an example of an approved antibody conjugate used for the treatment of leukemia.

Accordingly, it is another object of this invention to provide antigens (cancer-associated polypeptides) associated with a variety of cancers as targets for diagnostic and/or therapeutic antibodies. These antigens are also useful for drug discovery (e.g., small molecules) and for further characterization of cellular regulation, growth, and differentiation.

### SUMMARY OF THE INVENTION

In accordance with the objects outlined above, the present invention provides methods for screening for compositions that modulate cancer, especially lymphoma and leukemia. The present invention also provides methods for screening for compositions which modulate carcinomas, especially mammary adenocarcinomas. Also provided herein are methods of inhibiting proliferation of a cell, preferably a lymphoma cell or a breast cancer cell. Methods of treatment of cancer, including diagnosis, are also provided herein.

In one aspect, a method of screening drug candidates comprises providing a cell that expresses a cancer-associated (CA) gene or fragments thereof. Preferred embodiments of CA genes are genes that are differentially expressed in cancer cells, preferably lymphatic, breast, prostate or epithelial cells, compared to other cells. Preferred embodiments of CA genes used in the methods herein include, but are not limited to the nucleic acids selected from Tables 1-6. That is, they include but are not limited to PVT1, SNL1, CCND1, FOSB, MYC, or NFKB1. The methods further include adding a drug candidate to the cell and determining the effect of the drug candidate on the expression of the CA gene.

In one embodiment, the method of screening drug candidates includes comparing the level of expression in the absence of the drug candidate to the level of expression in the presence of the drug candidate.

Also provided herein is a method of screening for a bioactive agent capable of binding to a CA protein (CAP), the method comprising combining the CAP and a candidate bioactive agent, and determining the binding of the candidate agent to the CAP.

Further provided herein is a method for screening for a bioactive agent capable of modulating the activity of a CAP. In one embodiment, the method comprises combining the CAP and a candidate bioactive agent, and determining the effect of the candidate agent on the bioactivity of the CAP.

Also provided is a method of evaluating the effect of a candidate carcinoma drug comprising administering the drug to a patient and removing a cell sample from the patient. The expression profile of the cell is then determined. This method may further comprise comparing the expression profile of the patient to an expression profile of a heathy individual.

In a further aspect, a method for inhibiting the activity of an CA protein is provided. In one embodiment, the method comprises administering to a patient an inhibitor of a CA protein preferably selected from the group consisting of the sequences outlined in Tables 1-6 or their complements.

A method of neutralizing the effect of a CA protein, preferably a protein encoded by a nucleic acid selected from the group of sequences outlined in Tables 1-6, is also provided. Preferably, the method comprises contacting an agent specific for said protein with said protein in an amount sufficient to effect neutralization.

Moreover, provided herein is a biochip/microarray comprising a nucleic acid segment which encodes a CA protein, preferably selected from the sequences outlined in Tables 1-6. That is, they include but are not limited to genes including PVT1, SNL1, CCND1, FOSB, MYC, or NFKB1. In different embodiments, a microarray of the invention comprises one, two, three, four, five or more human sequences from Tables 1-6 including, but not limited to, sequences related to PVT1, SNL1, CCND1, FOSB, MYC, or NFKB1.

Also provided herein is a method for diagnosing or determining the propensity to: cancers, especially lymphoma or leukemia or carcinoma (including breast cancer) by sequencing at least one carcinoma or lymphoma gene of an individual. In yet another aspect of the invention, a method is provided for determining cancer including lymphoma and leukemia gene copy numbers in an individual.

Novel sequences associated with cancer are also provided herein. Other aspects of the invention will become apparent to the skilled artisan by the following description of the invention.

### BRIEF DESCRIPTION OF THE FIGURES

FIG. 1 depicts PCR amplification of host-provirus junction fragments.

FIG. 2 shows an example of average threshold cycle (CT) values for a housekeeper gene and target gene.

FIG. 3 shows an example of the calculated difference (ΔΔC_{T}) between the C_{T} values of target and housekeeper genes (ΔC_{T}) for various samples.

FIG. 4 shows the ΔΔC_{T} and comparative expression level for each sample from FIG. 3.

FIG. 5 depicts mRNA expression of SNL1 in breast cancer tissue compared with expression in normal tissue. Samples 1-50 are breast cancer samples. Samples 51 and 52 are normal tissues. Bars represent the mean of expression level. Error bars represent standard deviation.

FIG. 5 depicts mRNA expression of FOSB in colon cancer tissue compared with expression in normal tissue. Samples 1-11 are normal samples. Samples 12-31 are colon cancer tissues. Bars represent the mean of expression level. Error bars represent standard deviation.

FIG. 7 depicts mRNA expression of FOSB in lung cancer tissue compared with expression in normal tissue. Samples 1-9 are normal samples. Samples 10-43 are lung cancer tissues. Bars represent the mean of expression level. Error bars represent standard deviation.

FIG. 8 depicts mRNA expression of FOSB in pancreas cancer tissue compared with expression in normal tissue. Samples 1-10 are normal samples. Samples 11-31 are pancreas cancer tissues. Bars represent the mean of expression level. Error bars represent standard deviation.

FIG. 9 depicts mRNA expression of FOSB in ovary cancer tissue compared with expression in normal tissue. Samples 1-16 are normal samples. Samples 17-44 are ovary cancer tissues. Bars represent the mean of expression level. Error bars represent standard deviation.

FIG. 10 depicts mRNA expression of FOSB in stomach cancer tissue compared with expression in normal tissue. Samples 1-10 are normal samples. Samples 11-39 are stomach cancer tissues. Bars represent the mean of expression level. Error bars represent standard deviation.

FIG. 11 depicts mRNA expression of FOSB in breast cancer tissue compared with expression in normal tissue. Samples 1-9 are normal samples. Samples 10-30 are breast cancer tissues. Bars represent the mean of expression level. Error bars represent standard deviation.

FIG. 12 depicts mRNA expression of FOSB in prostate cancer tissue compared with expression in normal tissue. Samples 1-7 are normal samples. Samples 8-37 are prostate cancer tissues. Bars represent the mean of expression level. Error bars represent standard deviation.

FIG. 13 depicts mRNA expression of MYC in breast cancer tissue compared with expression in normal tissue. Samples 1-50 are breast cancer samples. Bars represent the mean of expression level. Error bars represent standard deviation.

FIG. 14 depicts DNA amplification of MYC in breast cancer tissue compared with expression in normal tissue. Samples 1-49 are breast cancer samples. Bars represent the mean of expression level. Error bars represent standard deviation.

FIG. 15 depicts mRNA expression of CCND1 in breast cancer tissue compared with expression in normal tissue. Samples 1-50 are breast cancer samples. Samples 51 and 52 are normal tissues. Bars represent the mean of expression level. Error bars represent standard deviation.

FIG. 16 depicts mRNA expression of CCND1 in colon cancer (sigmoid) tissue compared with expression in normal tissue. Samples 1-12 are normal samples. Samples 13-29 are colon cancer tissues. Bars represent the mean of expression level. Error bars represent standard deviation.

FIG. 17 depicts mRNA expression of CCND1 in colon cancer (transverse) tissue compared with expression in normal tissue. Samples 1-12 are normal samples. Samples 13-30 are colon cancer tissues. Bars represent the mean of expression level. Error bars represent standard deviation.

FIG. 18 depicts mRNA expression of CCND1 in lung tissue compared with expression in normal tissue. Samples 1-9 are normal samples. Samples 10-43 are lung cancer tissues. Bars represent the mean of expression level. Error bars represent standard deviation.

FIG. 19 depicts mRNA expression of CCND1 in ovary tissue compared with expression in normal tissue. Samples 1-14 are normal samples. Samples 15-41 are ovary cancer tissues. Bars represent the mean of expression level. Error bars represent standard deviation.

FIG. 20 depicts mRNA expression of NFKB1 in breast cancer tissue compared with expression in normal tissue. Samples 1-50 are breast cancer samples. Samples 51 and 52 are normal tissues. Bars represent the mean of expression level. Error bars represent standard deviation.

FIG. 21 depicts mRNA expression of NFKB1 in lung tissue compared with expression in normal tissue. Samples 1-9 are normal samples. Samples 10-44 are lung cancer tissues. Bars represent the mean of expression level. Error bars represent standard deviation.

FIG. 22 depicts mRNA expression of NFKB1 in skin tissue compared with expression in normal tissue. Samples 1-9 are normal samples. Samples 10-46 are skin cancer tissues. Bars represent the mean of expression level. Error bars represent standard deviation.

FIG. 23 depicts mRNA expression of PVT1 in breast cancer tissue compared with expression in normal tissue. Samples 1-50 are breast cancer samples. Bars represent the mean of expression level. Error bars represent standard deviation.

FIG. 24 depicts DNA amplification of PVT1 in breast cancer tissue compared with expression in normal tissue. Samples 1-50 are breast cancer samples. Bars represent the mean of expression level. Error bars represent standard deviation.

### DETAILED DESCRIPTION OF PREFERRED EMBODIMENTS

The present invention is directed to a number of sequences associated with cancers, especially lymphoma, breast cancer or prostate cancer. The relatively tight linkage between clonally-integrated proviruses and protooncogenes forms “provirus tagging”, in which slow-transforming retroviruses that act by an insertion mutation mechanism are used to isolate protooncogenes. In some models, uninfected animals have low cancer rates, and infected animals have high cancer rates. It is known that many of the retroviruses involved do not carry transduced host protooncogenes or pathogenic trans-acting viral genes, and thus the cancer incidence must therefore be a direct consequence of proviral integration effects into host protooncogenes. Since proviral integration is random, rare integrants will “activate” host protooncogenes that provide a selective growth advantage, and these rare events result in new proviruses at clonal stoichiometries in tumors. In contrast to mutations caused by chemicals, radiation, or spontaneous errors, protooncogene insertion mutations can be easily located by virtue of the fact that a convenient-sized genetic marker of known sequence (the provirus) is present at the site of mutation. Host sequences that flank clonally integrated proviruses can be cloned using a variety of strategies. Once these sequences are in hand, the tagged protooncogenes can be subsequently identified. The presence of provirus at the same locus in two or more independent tumors is prima facie evidence that a protooncogene is present at or very near the provirus integration sites. This is because the genome is too large for random integrations to result in observable clustering. Any clustering that is detected is unequivocal evidence for biological selection (i.e. the tumor phenotype). Moreover, the pattern of proviral integrants (including orientations) provides compelling positional information that makes localization of the target gene at each cluster relatively simple. The three mammalian retroviruses that are known to cause cancer by an insertion mutation mechanism are FeLV (leukemia/lymphoma in cats), MLV (leukemia/lymphoma in mice and rats), and MMTV (mammary cancer in mice).

Thus, the use of oncogenic retroviruses, whose sequences insert into the genome of the host organism resulting in cancer, allows the identification of host sequences involved in cancer. These sequences may then be used in a number of different ways, including diagnosis, prognosis, screening for modulators (including both agonists and antagonists), antibody generation (for immunotherapy and imaging), etc. However, as will be appreciated by those in the art, oncogenes that are identified in one type of cancer such as lymphoma or leukemia have a strong likelihood of being involved in other types of cancers as well. Thus, while the sequences outlined herein are initially identified as correlated with lymphoma, they can also be found in other types of cancers as well, outlined below.

Definitions

Accordingly, the present invention provides nucleic acid and protein sequences that are associated with cancer, herein termed “cancer associated” or “CA” sequences. In one embodiment, the present invention provides nucleic acid and protein sequences that are associated with cancers that originate in lymphatic tissue, herein termed “lymphoma associated,” “leukemia associated” or “LA” sequences. In another embodiment, the present invention provides nucleic acid and protein sequences that are associated with carcinomas which originate in breast tissue, herein termed “breast cancer associated” or “BCA” sequences.

Suitable cancers that can be diagnosed or screened for using the methods of the present invention include cancers classified by site or by histological type. Cancers classified by site include cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and, mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); lymphomas (Hodgkin's disease and non-Hodgkin's lymphoma), multiple myeloma, and leukemias (lymphocytic leukemia; myeloid leukemia; monocytic leukemia; and other leukemias).

Other cancers, classified by histological type, that may be associated with the sequences of the invention include, but are not limited to, Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Bronchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma w/ squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malig melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovarii, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleukemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia.

In addition, the CA genes may be involved in other diseases such as, but not limited to, diseases associated with aging or neurodegeneration.

“Association” in this context means that the nucleotide or protein sequences are either differentially expressed, activated, inactivated or altered in cancers as compared to normal tissue. As outlined below, CA sequences include those that are up-regulated (i.e. expressed at a higher level), as well as those that are down-regulated (i.e. expressed at a lower level), in cancers. CA sequences also include sequences that have been altered (i.e., truncated sequences or sequences with substitutions, deletions or insertions, including point mutations) and show either the same expression profile or an altered profile. In a preferred embodiment, the CA sequences are from humans; however, as will be appreciated by those in the art, CA sequences from other organisms may be useful in animal models of disease and drug evaluation; thus, other CA sequences are provided, from vertebrates, including mammals, including rodents (rats, mice, hamsters, guinea pigs, etc.), primates, and farm animals (including sheep, goats, pigs, cows, horses, etc). In some cases, prokaryotic CA sequences may be useful. CA sequences from other organisms may be obtained using the techniques outlined below.

CA sequences include both nucleic acid and amino acid sequences. In one embodiment, the CA sequences are recombinant nucleic acids. By the term “recombinant nucleic acid” herein is meant nucleic acid, originally formed in vitro, in general, by the manipulation of nucleic acid by polymerases and endonucleases, in a form not normally found in nature. Thus a recombinant nucleic acid is also an isolated nucleic acid, in a linear form, or cloned in a vector formed in vitro by ligating DNA molecules that are not normally joined, are both considered recombinant for the purposes of this invention. It is understood that once a recombinant nucleic acid is made and reintroduced into a host cell or organism, it will replicate using the in vivo cellular machinery of the host cell rather than in vitro manipulations; however, such nucleic acids, once produced recombinantly, although subsequently replicated in vivo, are still considered recombinant or isolated for the purposes of the invention. As used herein a “polynucleotide” or “nucleic acid” is a polymeric form of nucleotides of any length, either ribonucleotides or deoxyribonucleotides. This term refers only to the primary structure of the molecule. Thus, this term includes double- and single-stranded DNA and RNA. It also includes known types of modifications, for example, labels which are known in the art, methylation, “caps”, substitution of one or more of the naturally occurring nucleotides with an analog, internucleotide modifications such as, for example, those with uncharged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelators (e.g., metals, radioactive metals, etc.), those containing alkylators, those with modified linkages (e.g., alpha anomeric nucleic acids, etc.), as well as unmodified forms of the polynucleotide.

As used herein, a polynucleotide “derived from” a designated sequence refers to a polynucleotide sequence which is comprised of a sequence of approximately at least about 6 nucleotides, preferably at least about 8 nucleotides, more preferably at least about 10-12 nucleotides, and even more preferably at least about, 15-20 nucleotides corresponding to a region of the designated nucleotide sequence. “Corresponding” means homologous to or complementary to the designated sequence. Preferably, the sequence of the region from which the polynucleotide is derived is homologous to or complementary to a sequence that is unique to a CA gene.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises about 50-75% by weight of the total protein, with about 80% being preferred, and about 90% being particularly preferred. The definition includes the production of a CA protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

In a preferred embodiment, the CA sequences are nucleic acids. As will be appreciated by those in the art and is more fully outlined below, CA sequences are useful in a variety of applications, including diagnostic applications, which will detect naturally occurring nucleic acids, as well as screening applications; for example, biochips comprising nucleic acid probes to the CA sequences can be generated. In the broadest sense, use of “nucleic acid,” “polynucleotide” or “oligonucleotide” or equivalents herein means at least two nucleotides covalently linked together. In some embodiments, an oligonucleotide is an oligomer of 6, 8, 10, 12, 20, 30 or up to 100 nucleotides. A “polynucleotide” or “oligonucleotide” may comprise DNA, RNA, PNA or a polymer of nucleotides linked by phosphodiester and/or any alternate bonds.

A nucleic acid of the present invention generally contains phosphodiester bonds, although in some cases, as outlined below (for example, in antisense applications or when a nucleic acid is a candidate drug agent), nucleic acid analogs may have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done for a variety of reasons, for example to increase the stability and half-life of such molecules in physiological environments for use in anti-sense applications or as probes on a biochip.

As will be appreciated by those in the art, all of these nucleic acid analogs may find use in the present invention. In addition, mixtures of naturally occurring nucleic acids and analogs can be made; alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. As will be appreciated by those in the art, the depiction of a single strand “Watson” also defines the sequence of the other strand “Crick”; thus the sequences described herein also includes the complement of the sequence. The nucleic acid may be DNA, both genomic and cDNA, RNA, or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

As used herein, the term “tag,” “sequence tag” or “primer tag sequence” refers to an oligonucleotide with specific nucleic acid sequence that serves to identify a batch of polynucleotides bearing such tags therein. Polynucleotides from the same biological source are covalently tagged with a specific sequence tag so that in subsequent analysis the polynucleotide can be identified according to its source of origin. The sequence tags also serve as primers for nucleic acid amplification reactions.

A “microarray” is a linear or two-dimensional array of preferably discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide primers placed on a chip or other surfaces used to amplify or clone target polynucleotides. Since the position of each particular group of primers in the array is known, the identities of the target polynucleotides can be determined based on their binding to a particular position in the microarray.

A “linker” is a synthetic oligodeoxyribonucleotide that contains a restriction site. A linker may be blunt end-ligated onto the ends of DNA fragments to create restriction sites that can be used in the subsequent cloning of the fragment into a vector molecule.

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical, chemical, or any other appropriate means. The term “label” is used to refer to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The label may also be a compound that is a member of a binding pair, the other member of which bears a detectable physical property.

The term “support” refers to conventional supports such as beads, particles, dipsticks, fibers, filters, membranes, and silane or silicate supports such as glass slides.

The term “amplify” is used in the broad sense to mean creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases.

As used herein, a “biological sample” refers to a sample of tissue or fluid isolated from an individual, including but not limited to, for example, blood, plasma, serum, spinal fluid, lymph fluid, skin, respiratory, intestinal and genitourinary tracts, tears, saliva, milk, cells (including but not limited to blood cells), tumors, organs, and also samples of in vitro cell culture constituents.

The term “biological sources” as used herein refers to the sources from which the target polynucleotides are derived. The source can be of any form of “sample” as described above, including but not limited to, cell, tissue or fluid. “Different biological sources” can refer to different cells/tissues/organs of the same individual, or cells/tissues/organs from different individuals of the same species, or cells/tissues/organs from different species.

Cancer-Associated Sequences

The CA sequences of the invention were initially identified by infection of mice with a retrovirus such as murine leukemia virus (MLV) or mouse mammary tumor virus (MMTV) resulting in lymphoma. The CA sequences wre subsequently validated by determining expression levels of the corresponding gene product (e.g., mRNA) in breast cancer and other cancer tissue samples. Retroviruses have a genome that is made out of RNA. After a retrovirus infects a host cell, a double stranded DNA copy of the retrovirus genome (a “provirus”) is inserted into the genomic DNA of the host cell. The integrated provirus may affect the expression of host genes at or near the site of integration—a phenomenon known as retroviral insertional mutagenesis. Possible changes in the expression of host cell genes include: (i) increased expression of genes near the site of integration resulting from the proximity of elements in the provirus that act as transcriptional promoters and enhancers, (ii) functional inactivation of a gene caused by the integration of a provirus into the gene itself thus preventing the synthesis of a functional gene product, or (iii) expression of a mutated protein that has a different activity to the normal protein. Typically such a protein would be prematurely truncated and lack a regulatory domain near the C terminus. Such a protein might be constitutively active, or act as a dominant negative inhibitor of the normal protein. For example, retrovirus enhancers, including that of SL3-3, are known to act on genes up to approximately 200 kilobases from the insertion site. Moreover, many of these sequences are also involved in other cancers and disease states. Sequences of mouse genes according to this invention, that are identified in this manner are shown in Tables 1-6.

A CA sequence can be initially identified by substantial nucleic acid and/or amino acid sequence homology to the CA sequences outlined herein. Such homology can be based upon the overall nucleic acid or amino acid sequence, and is generally determined as outlined below, using either homology programs or hybridization conditions.

In one embodiment, CA sequences are up-regulated in cancers; that is, the expression of these genes is higher in cancer tissue as compared to normal tissue of the same differentiation stage. “Up-regulation” as used herein means increased expression by about 50%, preferably about 100%, more preferably about 150% to about 200%, with up-regulation from 300% to 1000% being preferred.

In another embodiment, CA sequences are down-regulated in cancers; that is, the expression of these genes is lower in cancer tissue as compared to normal tissue of the same differentiation stage. “Down-regulation” as used herein means decreased expression by about 50%, preferably about 100%, more preferably about 150% to about 200%, with down-regulation from 300% to 1000% to no expression being preferred.

In yet another embodiment, CA sequences are those that have altered sequences but show either the same or an altered expression profile as compared to normal lymphoid tissue of the same differentiation stage. “Altered CA sequences” as used herein also refers to sequences that are truncated, contain insertions or contain point mutations.

CA proteins of the present invention may be classified as secreted proteins, transmembrane proteins or intracellular proteins. In a preferred embodiment the CA protein is an intracellular protein. Intracellular proteins may be found in the cytoplasm and/or in the nucleus. Intracellular proteins are involved in all aspects of cellular function and replication (including, for example, signaling pathways); aberrant expression of such proteins results in unregulated or disregulated cellular processes. For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing intracellular proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate.

In a preferred embodiment, the CA sequences are transmembrane proteins. Transmembrane proteins are molecules that span the phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors are classified as “seven transmembrane domain” proteins, as they contain 7 membrane spanning regions. Important transmembrane protein receptors include, but are not limited to insulin receptor, insulin-like growth factor receptor, human growth hormone receptor, glucose transporters, transferrin receptor, epidermal growth factor receptor, low density lipoprotein receptor, leptin receptor, interleukin receptors, e.g. IL-1 receptor, IL-2 receptor, etc. CA proteins may be derived from genes that regulate apoptosis (IL-3, GM-CSF and Bcl-x) or are shown to have a role in the regulation of apoptosis.

Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted.

The extracellular domains of transmembrane proteins are diverse; however, conserved motifs are found repeatedly among various extracellular domains. Conserved structure and/or functions have been ascribed to different extracellular motifs. For example, cytokine receptors are characterized by a cluster of cysteines and a WSXWS (W=tryptophan, S=serine, X=any amino acid) motif. Immunoglobulin-like domains are highly conserved. Mucin-like domains may be involved in cell adhesion and leucine-rich repeats participate in protein-protein interactions.

Many extracellular domains are involved in binding to other molecules. In one aspect, extracellular domains are receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell for example via a glycosylphosphatidylinositol (GPI) anchor, or may themselves be transmembrane proteins. Extracellular domains also associate with the extracellular matrix and contribute to the maintenance of the cell structure.

CA proteins that are transmembrane are particularly preferred in the present invention as they are good targets for immunotherapeutics, as are described herein. In addition, as outlined below, transmembrane proteins can be also useful in imaging modalities.

It will also be appreciated by those in the art that a transmembrane protein can be made soluble by removing transmembrane sequences, for example through recombinant methods. Furthermore, transmembrane proteins that have been made soluble can be made to be secreted through recombinant means by adding an appropriate signal sequence.

In a preferred embodiment, the CA proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. CA proteins that are secreted proteins are particularly preferred in the present invention as they serve as good targets for diagnostic markers, for example for blood tests.

CA Sequences and Homologs

A CA sequence is initially identified by substantial nucleic acid and/or amino acid sequence homology to the CA sequences outlined herein. Such homology can be based upon the overall nucleic acid or amino acid sequence, and is generally determined as outlined below, using either homology programs or hybridization conditions.

As used herein, a nucleic acid is a “CA nucleic acid” if the overall homology of the nucleic acid sequence to one of the nucleic acids of Tables 1-6 is preferably greater than about 75%, more preferably greater than about 80%, even more preferably greater than about 85% and most preferably greater than 90%. In some embodiments the homology will be as high as about 93 to 95 or 98%. In a preferred embodiment, the sequences that are used to determine sequence identity or similarity are selected from those of the nucleic acids of Tables 1-6. In another embodiment, the sequences are naturally occurring allelic variants of the sequences of the nucleic acids of Tables 1-6. In another embodiment, the sequences are sequence variants as further described herein.

Homology in this context means sequence similarity or identity, with identity being preferred. A preferred comparison for homology purposes is to compare the sequence containing sequencing errors to the correct sequence. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

One example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters include a default gap weight of 3.00, a default gap length weight of 0.10, and weighted end gaps.

Another example of a useful algorithm is the BLAST (Basic Local Alignment Search Tool) algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460-480 (1996); http://blast.wustl.edu/]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A percent amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Thus, “percent (%) nucleic acid sequence identity” is defined as the percentage of nucleotide residues in a candidate sequence that are identical with the nucleotide residues of the nucleic acids of Tables 1-6. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer nucleotides than those of the nucleic acids of Tables 1-6, it is understood that the percentage of homology will be determined based on the number of homologous nucleosides in relation to the total number of nucleosides. Thus homology of sequences shorter than those of the sequences identified herein will be determined using the number of nucleosides in the shorter sequence.

In another embodiment of the invention, polynucleotide compositions are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC (“saline sodium citrate”; 9 mM NaCl, 0.9 mM sodium citrate), 0.5% SDS, 1.0 mM EDTA (pH 8.0); hybridizing at 50-60° C., 5×SSC, overnight; followed by washing twice at 65° C. for 20 minutes with each of 2×, 0.5× and 0.2×SSC containing 0.1%-SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C., or 65-70° C. Stringent conditions may also be achieved with the addition of destabilizing agents such as formamide.

Thus nucleic acids that hybridize under high stringency to the nucleic acids identified in the figures, or their complements, are considered CA sequences. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for longer probes (e.g. greater than 50 nucleotides). In another embodiment, less stringent hybridization conditions are used; for example, moderate or low stringency conditions may be used, as are known in the art; see Maniatis and Ausubel, supra, and Tijssen, supra.

In addition, the CA nucleic acid sequences of the invention are fragments of larger genes, i.e. they are nucleic acid segments. Alternatively, the CA nucleic acid sequences can serve as indicators of oncogene position, for example, the CA sequence may be an enhancer that activates a protooncogene. “Genes” in this context includes coding regions, non-coding regions, and mixtures of coding and non-coding regions. Accordingly, as will be appreciated by those in the art, using the sequences provided herein, additional sequences of the CA genes can be obtained, using techniques well known in the art for cloning either longer sequences or the full-length sequences; see Maniatis et al., and Ausubel, et al., supra, hereby expressly incorporated by reference. In general, this is done using PCR, for example, kinetic PCR.

Detection of CA Expression

Once the CA nucleic acid is identified, it can be cloned and, if necessary, its constituent parts recombined to form the entire CA nucleic acid. Once isolated from its natural source, e.g., contained within a plasmid or other vector or excised therefrom as a linear nucleic acid segment, the recombinant CA nucleic acid can be further used as a probe to identify and isolate other CA nucleic acids, for example additional coding regions. It can also be used as a “precursor” nucleic acid to make modified or variant CA nucleic acids and proteins. In a preferred embodiment, once a CA gene is identified its nucleotide sequence is used to design probes specific for the CA gene.

The CA nucleic acids of the present invention are used in several ways. In a first embodiment, nucleic acid probes hybridizable to CA nucleic acids are made and attached to biochips to be used in screening and diagnostic methods, or for gene therapy and/or antisense applications. Alternatively, the CA nucleic acids that include coding regions of CA proteins can be put into expression vectors for the expression of CA proteins, again either for screening purposes or for administration to a patient.

Recent developments in DNA microarray technology make it possible to conduct a large scale assay of a plurality of target CA nucleic acid molecules on a single solid phase support. U.S. Pat. No. 5,837,832 (Chee et al.) and related patent applications describe immobilizing an array of oligonucleotide probes for hybridization and detection of specific nucleic acid sequences in a sample. Target polynucleotides of interest isolated from a tissue of interest are hybridized to the DNA chip and the specific sequences detected based on the target polynucleotides' preference and degree of hybridization at discrete probe locations. One important use of arrays is in the analysis of differential gene expression, where the profile of expression of genes in different cells, often a cell of interest and a control cell, is compared and any differences in gene expression among the respective cells are identified. Such information is useful for the identification of the types of genes expressed in a particular cell or tissue type and diagnosis of cancer conditions based on the expression profile.

Typically, RNA from the sample of interest is subjected to reverse transcription to obtain labeled cDNA. See U.S. Pat. No. 6,410,229 (Lockhart et al.) The cDNA is then hybridized to oligonucleotides or cDNAs of known sequence arrayed on a chip or other surface in a known order. The location of the oligonucleotide to which the labeled cDNA hybridizes provides sequence information on the cDNA, while the amount of labeled hybridized RNA or cDNA provides an estimate of the relative representation of the RNA or(cDNA of interest. See Schena, et al. Science 270:467-470 (1995). For example, use of a cDNA microarray to analyze gene expression patterns in human cancer is described by DeRisi, et al (Nature Genetics 14:457-460 (1996)).

In a preferred embodiment, nucleic acid probes corresponding to CA nucleic acids (both the nucleic acid sequences outlined in the figures and/or the complements thereof) are made. Typically, these probes are synthesized based on the disclosed sequences of this invention. The nucleic acid probes attached to the biochip are designed to be substantially complementary to the CA nucleic acids, i.e. the target sequence (either the target sequence of the sample or to other probe sequences, for example in sandwich assays), such that specific hybridization of the target sequence and the probes of the present invention occurs. As outlined below, this complementarity need not be perfect, in that there may be any number of base pair mismatches that will interfere with hybridization between the target sequence and the single stranded nucleic acids of the present invention. It is expected that the overall homology of the genes at the nucleotide level probably will be about 40% or greater, probably about 60% or greater, and even more probably about 80% or greater; and in addition that there will be corresponding contiguous sequences of about 8-12 nucleotides or longer. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein. Whether or not a sequence is unique to a CA gene according to this invention can be determined by techniques known to those of skill in the art. For example, the sequence can be compared to sequences in databanks, e.g., GeneBank, to determine whether it is present in the uninfected host or other organisms. The sequence can also be compared to the known sequences of other viral agents, including those that are known to induce cancer.

A nucleic acid probe is generally single stranded but can be partly single and partly double stranded. The strandedness of the probe is dictated by the structure, composition, and properties of the target sequence. In general, the oligonucleotide probes range from about 6, 8, 10, 12, 15, 20, 30 to about 100 bases long, with from about 10 to about 80 bases being preferred, and from about 30 to about 50 bases being particularly preferred. That is, generally entire genes are rarely used as probes. In some embodiments, much longer nucleic acids can be used, up to hundreds of bases. The probes are sufficiently specific to hybridize to complementary template sequence under conditions known by those of skill in the art. The number of mismatches between the probes sequences and their complementary template (target) sequences to which they hybridize during hybridization generally do not exceed 15%, usually do not exceed 10% and preferably do not exceed 5%, as determined by FASTA (default settings).

Oligonucleotide probes can include the naturally-occurring heterocyclic bases normally found in nucleic acids (uracil, cytosine, thymine, adenine and guanine), as well as modified bases and base analogues. Any modified base or base analogue compatible with hybridization of the probe to a target sequence is useful in the practice of the invention. The sugar or glycoside portion of the probe can comprise deoxyribose, ribose, and/or modified forms of these sugars, such as, for example, 2′-O-alkyl ribose. In a preferred embodiment, the sugar moiety is 2′-deoxyribose; however, any sugar moiety that is compatible with the ability of the probe to hybridize to a target sequence can be used.

In one embodiment, the nucleoside units of the probe are linked by a phosphodiester backbone, as is well known in the art. In additional embodiments, internucleotide linkages can include any linkage known to one of skill in the art that is compatible with specific hybridization of the probe including, but not limited to phosphorothioate, methylphosphonate, sulfamate (e.g., U.S. Pat. No. 5,470,967) and polyamide (i.e., peptide nucleic acids). Peptide nucleic acids are described in Nielsen et al. (1991) Science 254: 1497-1500, U.S. Pat. No. 5,714,331, and Nielsen (1999) Curr. Opin. Biotechnol. 10:71-75.

In certain embodiments, the probe can be a chimeric molecule; i.e., can comprise more than one type of base or sugar subunit, and/or the linkages can be of more than one type within the same primer. The probe can comprise a moiety to facilitate hybridization to its target sequence, as are known in the art, for example, intercalators and/or minor groove binders. Variations of the bases, sugars, and internucleoside backbone, as well as the presence of any pendant group on the probe, will be compatible with the ability of the probe to bind, in a sequence-specific fashion, with its target sequence. A large number of structural modifications, both known and to be developed, are possible within these bounds. Advantageously, the probes according to the present invention may have structural characteristics such that they allow the signal amplification, such structural characteristics being, for example, branched DNA probes as those described by Urdea et al. (Nucleic Acids Symp. Ser., 24:197-200 (1991)) or in the European Patent No. EP-0225,807. Moreover, synthetic methods for preparing the various heterocyclic bases, sugars, nucleosides and nucleotides that form the probe, and preparation of oligonucleotides of specific predetermined sequence, are well-developed and known in the art. A preferred method for oligonucleotide synthesis incorporates the teaching of U.S. Pat. No. 5,419,966.

Multiple probes may be designed for a particular target nucleic acid to account for polymorphism and/or secondary structure in the target nucleic acid, redundancy of data and the like. In some embodiments, where more than one probe per sequence is used, either overlapping probes or probes to different sections of a single target CA gene are used. That is, two, three, four or more probes, with three being preferred, are used to build in a redundancy for a particular target. The probes can be overlapping (i.e. have some sequence in common), or specific for distinct sequences of a CA gene. When multiple target polynucleotides are to be detected according to the present invention, each probe or probe group corresponding to a particular target polynucleotide is situated in a discrete area of the microarray.

Probes may be in solution, such as in wells or on the surface of a micro-array, or attached to a solid support. Examples of solid support materials that can be used include a plastic, a ceramic, a metal, a resin, a gel and a membrane. Useful types of solid supports include plates, beads, magnetic material, microbeads, hybridization chips, membranes, crystals, ceramics and self-assembling monolayers. A preferred embodiment comprises a two-dimensional or three-dimensional matrix, such as a gel or hybridization chip with multiple probe binding sites (Pevzner et al., J. Biomol. Struc. & Dyn. 9:399-410, 1991; Maskos and Southern, Nuc. Acids Res. 20:1679-84, 1992). Hybridization chips can be used to construct very large probe arrays that are subsequently hybridized with a target nucleic acid. Analysis of the hybridization pattern of the chip can assist in the identification of the target nucleotide sequence. Patterns can be manually or computer analyzed, but it is clear that positional sequencing by hybridization lends itself to computer analysis and automation. Algorithms and software, which have been developed for sequence reconstruction, are applicable to the methods described herein (R. Drmanac et al., J. Biomol. Struc. & Dyn. 5:1085-1102, 1991; P. A. Pevzner, J. Biomol. Struc. & Dyn. 7:63-73, 1989).

As will be appreciated by those in the art, nucleic acids can be attached or immobilized to a solid support in a wide variety of ways. By “immobilized” herein is meant the association or binding between the nucleic acid probe and the solid support is sufficient to be stable under the conditions of binding, washing, analysis, and removal as outlined below. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as streptavidin, to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

Nucleic acid probes may be attached to the solid support by covalent binding such as by conjugation with a coupling agent or by, covalent or non-covalent binding such as electrostatic interactions, hydrogen bonds or antibody-antigen coupling, or by combinations thereof. Typical coupling agents include biotin/avidin, biotin/streptavidin, Staphylococcus aureus protein A/IgG antibody Fc fragment, and streptavidin/protein A chimeras (T. Sano and C. R. Cantor, Bio/Technology 9:1378-81 (1991)), or derivatives or combinations of these agents. Nucleic acids may be attached to the solid support by a photocleavable bond, an electrostatic bond, a disulfide bond, a peptide bond, a diester bond or a combination of these sorts of bonds. The array may also be attached to the solid support by a selectively releasable bond such as. 4,4′-dimethoxytrityl or its derivative. Derivatives which have been found to be useful include 3 or 4 [bis-(4-methoxyphenyl)]-methyl-benzoic acid, N-succinimidyl-3 or 4 [bis-(4-methoxyphenyl)]-methyl-benzoic acid, N-succinimidyl-3 or 4 [bis-(4-methoxyphenyl)]-hydroxymethyl-benzoic acid, N-succinimidyl-3 or 4 [bis-(4-methoxyphenyl)]-chloromethyl-benzoic acid, and salts of these acids.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. The solid phase support of the present invention can be of any solid materials and structures suitable for supporting nucleotide hybridization and synthesis. Preferably, the solid phase support comprises at least one substantially rigid surface on which the primers can be immobilized and the reverse transcriptase reaction performed. The substrates with which the polynucleotide microarray elements are stably associated may be fabricated from a variety of materials, including plastics, ceramics, metals, acrylamide, cellulose, nitrocellulose, glass, polystyrene, polyethylene vinyl acetate, polypropylene, polymethacrylate, polyethylene, polyethylene oxide, polysilicates, polycarbonates, Teflon®, fluorocarbons, nylon, silicon rubber, polyanhydrides, polyglycolic acid, polylactic acid, polyorthoesters, polypropylfumerate, collagen, glycosaminoglycans, and polyamino acids. Substrates may be two-dimensional or three-dimensional in form, such as gels, membranes, thin films, glasses, plates, cylinders, beads, magnetic beads, optical fibers, woven fibers, etc. A preferred form of array is a three-dimensional array. A preferred three-dimensional array is a collection of tagged beads. Each tagged bead has different primers attached to it. Tags are detectable by signaling means such as color (Luminex, Illumina) and electromagnetic field (Pharmaseq) and signals on tagged beads can even be remotely detected (e.g., using optical fibers). The size of the solid support can be any of the standard microarray sizes, useful for DNA microarray technology, and the size may be tailored to fit the particular machine being used to conduct a reaction of the invention. In general, the substrates allow optical detection and do not appreciably fluoresce.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference). In addition, in some cases, additional linkers, such as alkyl groups (including substituted and heteroalkyl groups) may be used.

In this embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside. In an additional embodiment, the immobilization to the solid support may be very strong, yet non-covalent. For example, biotinylated oligonucleotides can be made, which bind to surfaces covalently coated with streptavidin, resulting in attachment.

The arrays may be produced according to any convenient methodology, such as, preforming the polynucleotide microarray elements and then stably associating them with the surface. Alternatively, the oligonucleotides may be synthesized on the surface, as is known in the art. A number of different array configurations and methods for their production are known to those of skill in the art and disclosed in WO 95/25116 and WO 95/35505 (photolithographic techniques), U.S. Pat. No. 5,445,934 (in situ synthesis by photolithography), U.S. Pat. No. 5,384,261 (in situ synthesis by mechanically directed flow paths); and U.S. Pat. No. 5,700,637 (synthesis by spotting, printing or coupling); the disclosure of which are herein incorporated in their entirety by reference. Another method for coupling DNA to beads uses specific ligands attached to the end of the DNA to link to ligand-binding molecules attached to a bead. Possible ligand-binding partner pairs include biotin-avidin/streptavidin, or various antibody/antigen pairs such as digoxygenin-antidigoxygenin antibody (Smith et al., “Direct Mechanical Measurements of the Elasticity of Single DNA Molecules by Using Magnetic Beads,” Science 258:1122-1126 (1992)). Covalent chemical attachment of DNA to the support can be accomplished by using standard coupling agents to link the 5′-phosphate on the DNA to coated microspheres through a phosphoamidate bond. Methods for immobilization of oligonucleotides to solid-state substrates are well established. See Pease et al., Proc. Natl. Acad. Sci. USA 91(11):5022-5026 (1994). A preferred method of attaching oligonucleotides to solid-state substrates is described by Guo et al., Nucleic Acids Res. 22:5456-5465 (1994). Immobilization can be accomplished either by in situ DNA synthesis (Maskos and Southern, Nucleic Acids Research, 20:1679-1684 (1992) or by covalent attachment of chemically synthesized oligonucleotides (Guo et al., supra) in combination with robotic arraying technologies.

In addition to the solid-phase technology represented by biochip arrays, gene expression can also be quantified using liquid-phase arrays. One such system is kinetic polymerase chain reaction (PCR). Kinetic PCR allows for the simultaneous amplification and quantification of specific nucleic acid sequences. The specificity is derived from synthetic oligonucleotide primers designed to preferentially adhere to single-stranded nucleic acid sequences bracketing the target site. This pair of oligonucleotide primers form specific, non-covalently bound complexes on each strand of the target sequence. These complexes facilitate in vitro transcription of double-stranded DNA in opposite orientations. Temperature cycling of the reaction mixture creates a continuous cycle of primer binding, transcription, and re-melting of the nucleic acid to individual strands. The result is an exponential increase of the target dsDNA product. This product can be quantified in real time either through the use of an intercalating dye or a sequence specific probe. SYBR® Greene I, is an example of an intercalating dye, that preferentially binds to dsDNA resulting in a concomitant increase in the fluorescent signal. Sequence specific probes, such as used with TaqMan® technology, consist of a fluorochrome and a quenching molecule covalently bound to opposite ends of an oligonucleotide. The probe is designed to selectively bind the target DNA sequence between the two primers. When the DNA strands are synthesized during the PCR reaction, the fluorochrome is cleaved from the probe by the exonuclease activity of the polymerase resulting in signal dequenching. The probe signaling method can be more specific than the intercalating dye method, but in each case, signal strength is proportional to the dsDNA product produced. Each type of quantification method can be used in multi-well liquid phase arrays with each well representing primers and/or probes specific to nucleic acid sequences of interest. When used with messenger RNA preparations of tissues or cell lines, an array of probe/primer reactions can simultaneously quantify the expression of multiple gene products of interest. See Germer, S., et al., Genome Res. 10:258-266 (2000); Heid, C. A., et al., Genome Res. 6, 986-994 (1996).

Expression of CA Proteins

In a preferred embodiment, CA nucleic acids encoding CA proteins are used to make a variety of expression vectors to express CA proteins which can then be used in screening assays, as described below. The expression vectors may be either self-replicating extrachromosomal vectors or vectors which integrate into a host genome. Generally, these expression vectors include transcriptional and translational regulatory nucleic acid operably linked to the nucleic acid encoding the CA protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice. The transcriptional and translational regulatory nucleic acid will generally be appropriate to the host cell used to express the CA protein; for example, transcriptional and translational regulatory nucleic acid sequences from Bacillus are preferably used to express the CA protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression; and in a prokaryotic host for cloning and amplification. Furthermore, for integrating expression vectors, the expression vector contains at least one sequence homologous to the host cell genome, and preferably two homologous sequences that flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

The CA proteins of the present invention are produced by culturing a host cell transformed with an expression vector containing nucleic acid encoding a CA protein, under the appropriate conditions to induce or cause expression of the CA protein. The conditions appropriate for CA protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect, plant and animal cells, including mammalian cells. Of particular interest are Drosophila melanogaster cells, Saccharomyces cerevisiae and other yeasts, E. coli, Bacillus subtilis, Sf9 cells, C129 cells, 293 cells, Neurospora, BHK, CHO, COS, HeLa cells, THP1 cell line (a macrophage cell line) and human cells and cell lines.

In a preferred embodiment, the CA proteins are expressed in mammalian cells. Mammalian expression systems are also known in the art, and include retroviral systems. A preferred expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. Examples of transcription terminator and polyadenylation signals include those derived form SV40.

The methods of introducing exogenous nucleic acid into mammalian hosts, as well as other hosts, are well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, CA proteins are expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the CA protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes that render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

In one embodiment, CA proteins are produced in insect cells. Expression vectors for the transformation of insect cells, and in particular, baculovirus-based expression vectors, are well known in the art.

In a preferred embodiment, CA protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The CA protein may also be made as a fusion protein, using techniques well known in the art. Thus, for example, for the creation of monoclonal antibodies. If the desired epitope is small, the CA protein may be fused to a carrier protein to form an immunogen. Alternatively, the CA protein may be made as a fusion protein to increase expression, or for other reasons. For example, when the CA protein is a CA peptide, the nucleic acid encoding the peptide may be linked to other nucleic acid for expression purposes.

In one embodiment, the CA nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the CA nucleic acids, proteins and antibodies at any position. For example, the label should be capable of producing, either directly or indirectly, a detectable signal. The detectable moiety may be a radioisotope, such as ³H, ¹⁴C, ₃₂P, ³⁵S, or ¹²⁵I, a fluorescent or chemiluminescent compound, such as fluorescein: isothiocyanate, rhodamine, or luciferin, or an enzyme, such as alkaline phosphatase, beta-galactosidase or horseradish peroxidase. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Accordingly, the present invention also provides CA protein sequences. A CA protein of the present invention may be identified in several ways. “Protein” in this sense includes proteins, polypeptides, and peptides. As will be appreciated by those in the art, the nucleic acid sequences of the invention can be used to generate protein sequences. There are a variety of ways to do this, including cloning the entire gene and verifying its frame and amino acid sequence, or by comparing it to known sequences to search for homology to provide a frame, assuming the CA protein has homology to some protein in the database being used. Generally, the nucleic acid sequences are input into a program that will search all three frames for homology. This is done in a preferred embodiment using the following NCBI Advanced BLAST parameters. The program is blastx or blastn. The database is nr. The input data is as “Sequence in FASTA format”. The organism list is “none”. The “expect” is 10; the filter is default. The “descriptions” is 500, the “alignments” is 500, and the “alignment view” is pairwise. The “query Genetic Codes” is standard (1). The matrix is BLOSUM 62; gap existence cost is 11, per residue gap cost is 1; and the lambda ratio is 0.85 default. This results in the generation of a putative protein sequence.

In general, the term “polypeptide” as used herein refers to both the full-length polypeptide encoded by the recited polynucleotide, the polypeptide encoded by the gene represented by the recited polynucleotide, as well as portions or fragments thereof. The present invention encompasses variants of the naturally occurring proteins, wherein such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant polypeptides have a sequence that has at least about 80%, at least about 81%, at least about 82%, at least about 83%, at least about 84%, at least about 85%, at least about 86%, at least about 87%, at least about 88%, at least about 89%, usually at least about 90%, at least 91%, at least 92%, at least 93%, at least 94%, at least 95%, at least 96%, at least 97%, at least 98% and more usually at least about 99% sequence identity with a differentially expressed polypeptide described herein, as determined by the Smith-Waterman homology search algorithm using an affine gap search with a gap open penalty of 12 and a gap extension penalty of 2, BLOSUM matrix of 62. The Smith-Waterman homology search algorithm is taught in Smith and Waterman, Adv. Appl. Math. (1981) 2: 482-489. The variant polypeptides can be naturally or non-naturally glycosylated, i.e., the polypeptide has a glycosylation pattern that differs from the glycosylation pattern found in the corresponding naturally occurring protein.

Also within the scope of the invention are variants. Variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the protein (e.g., a functional domain and/or, where the polypeptide is a member of a protein family, a region associated with a consensus sequence). Selection of amino acid alterations for production of variants can be based upon the accessibility (interior vs. exterior) of the amino acid (see, e.g., Go et al, Int. J. Peptide Protein Res. (1980) 15:211), the thermostability of the variant polypeptide (see, e.g., Querol et al., Prot. Eng. (1996) 9:265), desired glycosylation sites (see, e.g., Olsen and Thomsen, J. Gen. Microbiol. (1991) 137:579), desired disulfide bridges (see, e.g., Clarke et al., Biochemistry (1993) 32:4322; and Wakarchuk et al., Protein Eng. (1994) 7:1379), desired metal binding sites (see, e.g., Toma et al., Biochemistry (1991) 30:97, and Haezerbrouck et al., Protein Eng. (1993) 6:643), and desired substitutions within proline loops (see, e.g., Masul et al., Appl. Env. Microbiol. (1994) 60:3579). Cysteine-depleted muteins can be produced as disclosed in U.S. Pat. No. 4,959,314.

Variants also include fragments of the polypeptides disclosed herein, particularly biologically active fragments and/or fragments corresponding to functional domains. Fragments of interest will typically be at least about 8 amino acids (aa) 10 aa, 15 aa, 20 aa, 25 aa, 30 aa, 35 aa, 40 aa, to at least about 45 aa in length, usually at least about 50 aa in length, at least about 75 aa, at least about 100 aa, at least about 125 aa, at least about 150 aa in length, at least about 200 aa, at least about 300 aa, at least about 400 aa and can be as long as 500 aa in length or longer, but will usually not exceed about 1000 aa in length, where the fragment will have a stretch of amino acids that is identical to a polypeptide encoded by a polynucleotide having a sequence of any one of the polynucleotide sequences provided herein, or a homolog thereof. The protein variants described herein are encoded by polynucleotides that are within the scope of the invention. The genetic code can be used to select the appropriate codons to construct the corresponding variants.

While altered expression of the polynucleotides associated with cancer is observed, altered levels of expression of the polypeptides encoded by these polynucleotides may likely play a role in cancers.

Also included within one embodiment of CA proteins are amino acid variants of the naturally occurring sequences, as determined herein. Preferably, the variants are preferably greater than about 75% homologous to the wild-type sequence, more preferably greater than about 80%, even more preferably greater than about 85% and most preferably greater than 90%. The present application is also directed to proteins containing polypeptides at least 80%, 85%, 90%, 95%, 96%, 97%, 98% or 99% identical to a CA polypeptide sequence set forth herein. As for nucleic acids, homology in this context means sequence similarity or identity, with identity being preferred. This homology will be determined using standard techniques known in the art as are outlined above for the nucleic acid homologies.

CA proteins of the present invention may be shorter or longer than the wild type amino acid sequences. Thus, in a preferred embodiment, included within the definition of CA proteins are portions or fragments of the wild type sequences herein. In addition, as outlined above, the CA nucleic acids of the invention may be used to obtain additional coding regions, and thus additional protein sequence, using techniques known in the art.

In a preferred embodiment, the CA proteins are derivative or variant CA proteins as compared to the wild-type sequence. That is, as outlined more fully below, the derivative CA peptide will contain at least one amino acid substitution, deletion or insertion, with amino acid substitutions being particularly preferred. The amino acid substitution, insertion or deletion may occur at any residue within the CA peptide.

Also included in an embodiment of CA proteins of the present invention are amino acid sequence variants. These variants fall into one or more of three classes: substitutional, insertional or deletional variants. These variants ordinarily are prepared by site-specific mutagenesis of nucleotides in the DNA encoding the CA protein, using cassette or PCR mutagenesis or other techniques well known in the art, to produce DNA encoding the variant, and thereafter expressing the DNA in recombinant cell culture as outlined above. However, variant CA protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the CA protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed CA variants screened for the optimal combination of desired activity. Techniques for making substitution mutations at predetermined sites in DNA having a known sequence are well known, for example, M13 primer mutagenesis and LAR mutagenesis. Screening of the mutants is done using assays of CA protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the CA protein are desired, substitutions are generally made in accordance with the following chart:

CHART 1

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made full length to more significantly affect one or more of the following: the structure of the polypeptide backbone in the area of the alteration (e.g., the alpha-helical or beta-sheet structure); the charge or hydrophobicity of the molecule at the target site; and the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the CA proteins as needed. Alternatively, the variant may be designed such that the biological activity of the CA protein is altered. For example, glycosylation sites may be altered or removed, dominant negative mutations created, etc.

Covalent modifications of CA polypeptides are included within the scope of this invention, for example for use in screening. One type of covalent modification includes reacting targeted amino acid residues of a CA polypeptide with an organic derivatizing agent that is capable of reacting with selected side chains or the N- or C-terminal residues of a CA polypeptide. Derivatization with bifunctional agents is useful, for instance, for crosslinking CA polypeptides to a water-insoluble support matrix or surface for use in the method for purifying anti-CA antibodies or screening assays, as is more fully described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the a-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the CA polypeptide included within the scope of this invention comprises altering the native glycosylation pattern of the polypeptide. “Altering the native glycosylation pattern” is intended for purposes herein to mean deleting one or more carbohydrate moieties found in native sequence CA polypeptide, and/or adding one or more glycosylation sites that are not present in the native sequence CA polypeptide.

Addition of glycosylation sites to CA polypeptides may be accomplished by altering the amino acid sequence thereof. The alteration may be made, for example, by the addition of, or substitution by, one or more serine or threonine residues to the native sequence CA polypeptide (for O-linked glycosylation sites). The CA amino acid sequence may optionally be altered through changes at the DNA level, particularly by mutating the DNA encoding the CA polypeptide at preselected bases such that codons are generated that will translate into the desired amino acids.

Another means of increasing the number of carbohydrate moieties on the CA polypeptide is by chemical or enzymatic coupling of glycosides to the polypeptide. Such methods are described in the art, e.g., in WO 87/05330 published Sep. 11, 1987, and in Aplin and Wriston, LA Crit. Rev. Biochem., pp. 259-306 (1981).

Removal of carbohydrate moieties present on the CA polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of CA comprises linking the CA polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

CA polypeptides of the present invention may also be modified in a way to form chimeric molecules comprising a CA polypeptide fused to another, heterologous polypeptide or amino acid sequence. In one embodiment, such a chimeric molecule comprises a fusion of a CA polypeptide with a tag polypeptide that provides an epitope to which an anti-tag antibody can selectively bind. The epitope tag is generally placed at the amino-or carboxyl-terminus of the CA polypeptide, although internal fusions may also be tolerated in some instances. The presence of such epitope-tagged forms of a CA polypeptide can be detected using an antibody against the tag polypeptide. Also, provision of the epitope tag enables the CA polypeptide to be readily purified by affinity purification using an anti-tag antibody or another type of affinity matrix that binds to the epitope tag. In an alternative embodiment, the chimeric molecule may comprise a fusion of a CA polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Also included with the definition of CA protein in one embodiment are other CA proteins of the CA family, and CA proteins from other organisms, which are cloned and expressed as outlined below. Thus, probe or degenerate polymerase chain reaction (PCR) primer sequences may be used to find other related CA proteins from humans or other organisms. As will be appreciated by those in the art, particularly useful probe and/or PCR primer sequences include the unique areas of the CA nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

In addition, as is outlined herein, CA proteins can be made that are longer than those encoded by the nucleic acids of the figures, for example, by the elucidation of additional sequences, the addition of epitope or purification tags, the addition of other fusion sequences, etc.

CA proteins may also be identified as being encoded by CA nucleic acids. Thus, CA proteins are encoded by nucleic acids that will hybridize to the sequences of the sequence listings, or their complements, as outlined herein.

CA Antigens and Antibodies Thereto

In one embodiment, the invention provides CA specific antibodies. In a preferred embodiment, when the CA protein is to be used to generate antibodies, for example for immunotherapy, the CA protein should share at least one epitope or determinant with the full-length protein. By “epitope” or “determinant” herein is meant a portion of a protein that will generate and/or bind an antibody or T-cell receptor in the context of MHC. Thus, in most instances, antibodies made to a smaller CA protein will be able to bind to the full-length protein. In a preferred embodiment, the epitope is unique; that is, antibodies generated to a unique epitope show little or no cross-reactivity.

Any polypeptide sequence encoded by the CA polynucleotide sequences may be analyzed to determine certain preferred regions of the polypeptide. Regions of high antigenicity are determined from data by DNASTAR analysis by choosing values that represent regions of the polypeptide that are likely to be exposed on the surface of the polypeptide in an environment in which antigen recognition may occur in the process of initiation of an immune response. For example, the amino acid sequence of a polypeptide encoded by a CA polynucleotide sequence may be analyzed using the default parameters of the DNASTAR computer algorithm (DNASTAR, Inc., Madison, Wis.; http://www.dnastar.com/).

Polypeptide features that may be routinely obtained using the DNASTAR computer algorithm include, but are not limited to, Garnier-Robson alpha-regions, beta-regions, turn-regions, and coil-regions (Gamier et al. J. Mol. Biol., 120: 97 (1978)); Chou-Fasman alpha-regions, beta-regions, and turn-regions (Adv. in Enzymol., 47:45-148 (1978)); Kyte-Doolittle hydrophilic regions and hydrophobic regions (J. Mol. Biol., 157:105-132 (1982)); Eisenberg alpha- and beta-amphipathic regions; Karplus-Schulz flexible regions; Emini surface-forming regions (J. Virol., 55(3):836-839 (1985)); and Jameson-Wolf regions of high antigenic index (CABIOS, 4(1):181-186 (1988)). Kyte-Doolittle hydrophilic regions and hydrophobic regions, Emini surface-forming regions, and Jameson-Wolf regions of high antigenic index (i.e., containing four or more contiguous amino acids having an antigenic index of greater than or equal to 1.5, as identified using the default parameters of the Jameson-Wolf program) can routinely be used to determine polypeptide regions that exhibit a high degree of potential for antigenicity. One approach for preparing antibodies to a protein is the selection and preparation of an amino acid sequence of all or part of the protein, chemically synthesizing the sequence and injecting it into an appropriate animal, typically a rabbit, hamster or a mouse. Oligopeptides can be selected as candidates for the production of an antibody to the CA protein based upon the oligopeptides lying in hydrophilic regions, which are thus likely to be exposed in the mature protein. Additional oligopeptides can be determined using, for example, the Antigenicity Index, Welling, G. W. et al., FEBS Lett. 188:215-218 (1985), incorporated herein by reference.

In one embodiment, the term “antibody” includes antibody fragments, as are known in the art, including Fab, Fab₂, single chain antibodies (Fv for example), chimeric antibodies, etc., either produced by the modification of whole antibodies or those synthesized de novo using recombinant DNA technologies.

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1-6, or fragment thereof or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Monoclonal antibody technology is used in implementing research, diagnosis and therapy. Monoclonal antibodies are used in radioimmunoassays, enzyme-linked immunosorbent assays, immunocytopathology, and flow cytometry for in vitro diagnosis, and in vivo for diagnosis and immunotherapy of human disease. Waldmann, T. A. (1991) Science 252:1657-1662. In particular, monoclonal antibodies have been widely applied to the diagnosis and therapy of cancer, wherein it is desirable to target malignant lesions while avoiding normal tissue. See, e.g., U.S. Pat. No. 4,753,894 to Frankel, et al.; U.S. Pat. No. 4,938,948 to Ring et al.; and U.S. Pat. No. 4,956,453 to Bjorn et al.

In one embodiment, the antibodies are bispecific antibodies. Bispecific antibodies are monoclonal, preferably human or humanized, antibodies that have binding specificities for at least two different antigens. A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European-Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients. In the present case, one of the binding specificities is for a protein encoded by a nucleic acid of Tables 1-6, or a fragment thereof, the other one is for any other antigen, and preferably for a cell-surface protein or receptor or receptor subunit, preferably one that is tumor specific.

In a preferred embodiment, the antibodies to CA are capable of reducing or eliminating the biological function of CA, as is described below. That is, the addition of anti-CA antibodies (either polyclonal or preferably monoclonal) to CA (or cells containing CA) may reduce or eliminate the CA activity. Generally, at least a 25% decrease in activity is preferred, with at least about 50% being particularly preferred and about a 95-100% decrease being especially preferred.

In a preferred embodiment the antibodies to the CA proteins are humanized antibodies. “Humanized” antibodies refer to a molecule having an antigen binding site that is substantially derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) that typically originate from different species. Typically, in these chimeric antibodies, the variable region of both light and heavy chains mimics the variable regions of antibodies derived from one species of mammals, while the constant portions are homologous to the sequences in antibodies derived from another. Most typically, chimeric antibodies comprise human and murine antibody fragments, generally human constant and mouse variable regions. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework residues (FR) regions are those of a human immunoglobulin consensus sequence. The humanized antibody optimally also will comprise at least a portion of an immunoglobulin constant region (Fe), typically that of a human immunoglobulin (Jones et al., Nature, 321:522-525 (1986); Riechmann et al., Nature, 332:323-329 (1988); and Presta, Curr. Op. Struct. Biol., 2:593-596 (1992)). One clear advantage to such chimeric forms is that, for example, the variable regions can conveniently be derived from presently known sources using readily available hybridomas or B cells from non human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However; the definition is not limited to this particular example.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy. Methods for humanizing non-human antibodies are well known in the art. Generally, a humanized antibody has one or more amino acid residues introduced into it from a source that is non-human. These non-human amino acid residues are often referred to as import residues, which are typically taken from an import variable domain. Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies [Cole et al., Monoclonal Antibodies and Cancer Therapy, Alan R. Liss, p. 77 (1985) and Boerner et al., J. Immunol., 147(1):86-95 (1991)]. Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995); Jones et al., Nature 321:522-525 (1986); Morrison et al., Proc. Natl. Acad. Sci, US.A., 81:6851-6855 (1984); Morrison and Oi, Adv. Immunol., 44:65-92 (1988); Verhoeyer et al., Science 239:1534-1536 (1988); Padlan, Molec. Immun. 28:489-498 (1991); Padlan, Molec. Immunol. 31(3):169-217 (1994); and Kettleborough, C. A. et al., Protein Eng. 4(7):773-83 (1991) each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept, of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu. One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region that disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g, via Ashwell receptors. See, e.g., U.S. Pat. Nos. 5,530,101 and 5,585,089 which are incorporated herein by reference.

Humanized antibodies to CA polypeptides can also be produced using transgenic animals that are engineered to contain human immunoglobulin loci. For example, WO 98/24893 discloses transgenic-animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. The monoclonal antibodies can be tested for the ability to inhibit or neutralize the biological activity or physiological effect of the corresponding protein.

In the present invention, CA polypeptides of the invention and variants thereof are used to immunize a transgenic animal as described above. Monoclonal antibodies are made using methods known in the art, and the specificity of the antibodies is tested using isolated CA polypeptides. Methods for preparation of the human or primate CA or an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld, J. Am. Chem. Soc. 85:2149, 1963 which is incorporated by reference) or the FMOC strategy on a Rapid Automated Multiple Peptide Synthesis system (E. I. du Pont de Nemours Company, Wilmington, Del.) (Caprino and Han, J. Org. Chem. 37:3404, 1972 which is incorporated by reference).

Polyclonal antibodies can be prepared by immunizing rabbits or other animals by injecting antigen followed by subsequent boosts at appropriate intervals. The animals are bled and sera assayed against purified CA proteins usually by ELISA or by bioassay based upon the ability to block the action of CA proteins. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. (Milstein and Kohler, Nature, 256:495-497, 1975; Gulfre and Milstein, Methods in Enzymology: Immunochemical Techniques 73:1-46, Langone and Banatis eds., Academic Press, 1981 which are incorporated by reference). The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of a CA polypeptide by treatment of a patient with specific antibodies to the CA protein.

Specific antibodies, either polyclonal or monoclonal, to the CA proteins can be produced by any suitable method known in the art as discussed above. For example, murine or human monoclonal antibodies can be produced by hybridoma technology or, alternatively, the CA proteins, or an immunologically active fragment thereof, or an anti-idiotypic antibody, or fragment thereof can be administered to an animal to elicit the production of antibodies capable of recognizing and binding to the CA proteins. Such antibodies can be from any class of antibodies including, but not limited to IgG, IgA, IgM, IgD, and IgE or in the case of avian species, IgY and from any subclass of antibodies.

By immunotherapy is meant treatment of a cancer with an antibody raised against a CA protein. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen.

In a preferred embodiment, oncogenes which encode secreted growth factors may be inhibited by raising antibodies against CA proteins that are secreted proteins as described above. Without being bound by theory, antibodies used for treatment, bind and prevent the secreted protein from binding to its receptor, thereby inactivating the secreted CA protein.

In another preferred embodiment, the CA protein to which antibodies are raised is a transmembrane protein. Without being bound by theory, antibodies used for treatment, bind the extracellular domain of the CA protein and prevent it from binding to other proteins, such as circulating ligands or cell-associated molecules. The antibody may cause down-regulation of the transmembrane CA protein. As will be appreciated by one of ordinary skill in the art, the antibody may be a competitive, non-competitive or uncompetitive inhibitor of protein binding to the extracellular domain of the CA protein. The antibody is also an antagonist of the CA protein. Further, the antibody prevents activation of the transmembrane CA protein. In one aspect, when the antibody prevents the binding of other molecules to the CA protein, the antibody prevents growth of the cell. The antibody may also sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity. Thus, cancers may be treated by administering to a patient antibodies directed against the transmembrane CA protein.

In another preferred embodiment, the antibody is conjugated to a therapeutic moiety. In one aspect the therapeutic moiety is a small molecule that modulates the activity of the CA protein. In another aspect the therapeutic moiety modulates the activity of molecules associated with or in close proximity to the CA protein. The therapeutic moiety may inhibit enzymatic activity such as protease or protein kinase activity associated with cancer.

In a preferred embodiment, the therapeutic moiety may also be a cytotoxic agent. In this method, radioisotopes, natural toxins, chemotherapy agents, or other substances (such as biological response modifiers) are chemically linked or conjugated to a monoclonal antibody to form “immunoconjugates” and “immunotoxins” which target the cytotoxic agent to tumor tissue or cells resulting in a reduction in the number of afflicted cells, thereby reducing symptoms associated with cancers, including lymphoma. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against CA proteins, or binding of a radionuclide to a chelating agent that has been covalently attached to the antibody. Targeting the therapeutic moiety to transmembrane CA proteins not only serves to increase the local concentration of therapeutic moiety in the cancer of interest, i.e., lymphoma, but also serves to reduce deleterious side effects that may be associated with the therapeutic moiety. A number of investigators have used monoclonal antibodies as carriers of cytotoxic substances in attempts to selectively direct those agents to malignant tissue. More particularly, a number of monoclonal antibodies have been conjugated to toxins such as ricin, abrin, diphtheria toxin and Pseudomonas exotoxin or to enzymatically active portions (A chains) thereof via heterobifunctional agents. See, e.g., U.S. Pat. No. 4,753,894 to Frankel et al.; Nevelle, et al. (1982) Immunol Rev 62:75-91; Ross et al. (1980) Eur. J Biochem 104; Vitteta et al. (1982) Immunol Rev 62:158-183; Raso et al. (1982) Cancer Res 42:457-464, and Trowbridge et al. (1981) Nature 294:171-173.

In another preferred embodiment, the CA protein against which the antibodies are raised is an intracellular protein. In this case, the antibody may be conjugated to a protein that facilitates entry into the cell. In one case, the antibody enters the cell by endocytosis. In another embodiment, a nucleic acid encoding the antibody is administered to the individual or cell. Moreover, wherein the CA protein can be targeted within a cell, e.g., the nucleus, an antibody thereto contains a signal for that target localization, e.g., a nuclear localization signal.

The CA antibodies of the invention specifically bind to CA proteins. By “specifically bind” herein is meant that the antibodies bind to the protein with a binding constant in the range of 10⁻⁴-10⁻⁶ M⁻¹, with a preferred range being 10⁻⁷-10⁻⁹ M⁻¹.

In a preferred embodiment, the CA protein is purified or isolated after expression. CA proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the CA protein may be purified using a standard anti-CA antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the CA protein. In some instances no purification will be necessary.

Detection of Cancer Phenotype

Once expressed and purified if necessary, the CA proteins and nucleic acids are useful in a number of applications. In one aspect, the expression levels of genes are determined for different cellular states in the cancer phenotype; that is, the expression levels of genes in normal tissue and in cancer tissue (and in some cases, for varying severities of lymphoma that relate to prognosis, as outlined below) are evaluated to provide expression profiles. An expression profile of a particular cell state or point of development is essentially a “fingerprint” of the state; while two states may have any particular gene similarly expressed, the evaluation of a number of genes simultaneously allows the generation of a gene expression profile that is unique to the state of the cell. By comparing expression profiles of cells in different states, information regarding which genes are important (including both up- and down-regulation of genes) in each of these states is obtained. Then, diagnosis may be done or confirmed: does tissue from a particular patient have the gene expression profile of normal or cancer tissue.

“Differential expression,” or equivalents used herein, refers to both qualitative as well as quantitative differences in the temporal and/or cellular expression patterns of genes, within and among the cells. Thus, a differentially expressed gene can qualitatively have its expression altered, including an activation or inactivation, in, for example, normal versus cancer tissue. That is, genes may be turned on or turned off in a particular state, relative to another state. As is apparent to the skilled artisan, any comparison of two or more states can be made. Such a qualitatively regulated gene will exhibit an expression pattern within a state or cell type which is detectable by standard techniques in one such state or cell type, but is not detectable in both. Alternatively, the determination is quantitative in that expression is increased or decreased; that is, the expression of the gene is either up-regulated, resulting in an increased amount of transcript, or down-regulated, resulting in a decreased amount of transcript. The degree to which expression differs need only be large enough to quantify via standard characterization techniques as outlined below, such as by use of Affymetrix GeneChip® expression arrays, Lockhart, Nature Biotechnology, 14:1675-1680 (1996), hereby expressly incorporated by reference. Other techniques include, but are not limited to, quantitative reverse transcriptase PCR, Northern analysis and RNase protection. As outlined above, preferably the change in expression (i.e. upregulation or downregulation) is at least about 50%, more preferably at least about 100%, more preferably at least about 150%, more preferably, at least about 200%, with from 300 to at least 1000% being especially preferred.

As will be appreciated by those in the art, this may be done by evaluation at either the gene transcript, or the protein level; that is, the amount of gene expression may be monitored using nucleic acid probes to the DNA or RNA equivalent of the gene transcript, and the quantification of gene expression levels, or, alternatively, the final gene product itself (protein) can be monitored, for example through the use of antibodies to the CA protein and standard immunoassays (ELISAs, etc.) or other techniques, including mass spectroscopy assays, 2D gel electrophoresis assays, etc. Thus, the proteins corresponding to CA genes, i.e. those identified as being important in a particular cancer phenotype, i.e., lymphoma, can be evaluated in a diagnostic test specific for that cancer.

In a preferred embodiment, gene expression monitoring is done and a number of genes, i.e. an expression profile, is monitored simultaneously, although multiple protein expression monitoring can be done as well. Similarly, these assays may be done on an individual basis as well.

In this embodiment, the CA nucleic acid probes may be attached to biochips as outlined herein for the detection and quantification of CA sequences in a particular cell. The assays are done as is known in the art. As will be appreciated by those in the art, any number of different CA sequences may be used as probes, with single sequence assays being used in some cases, and a plurality of the sequences described herein being used in other embodiments. In addition, while solid-phase assays are described, any number of solution based assays may be done as well.

In a preferred embodiment, both solid and solution based assays may be used to detect CA sequences that are up-regulated or down-regulated in cancers as compared to normal tissue. In instances where the CA sequence has been altered but shows the same expression profile or an altered expression profile, the protein will be detected as outlined herein.

In a preferred embodiment nucleic acids encoding the CA protein are detected. Although DNA or RNA encoding the CA protein may be detected, of particular interest are methods wherein the mRNA encoding a CA protein is detected. The presence of mRNA in a sample is an indication that the CA gene has been transcribed to form the mRNA, and suggests that the protein is expressed. Probes to detect the mRNA can be any nucleotide/deoxynucleotide probe that is complementary to and base pairs with the mRNA and includes but is not limited to oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a CA protein is detected by binding the digoxygenin with an anti-digoxygenin secondary antibody and developed with nitro blue tetrazolium and 5-bromo-4-chloro-3-indoyl phosphate.

In a preferred embodiment, any of the three classes of proteins as described herein (secreted, transmembrane or intracellular proteins) are used in diagnostic assays. The CA proteins, antibodies, nucleic acids, modified proteins and cells containing CA sequences are used in diagnostic assays. This can be done on an individual gene or corresponding polypeptide level, or as sets of assays.

As described and defined herein, CA proteins find use as markers of cancers, including lymphomas such as, but not limited to, Hodgkin's and non-Hodgkin's lymphoma. Detection of these proteins in putative cancer tissue or patients allows for a determination or diagnosis of the type of cancer. Numerous methods known to those of ordinary skill in the art find use in detecting cancers. In one embodiment, antibodies are used to detect CA proteins. A preferred method separates proteins from a sample or patient by electrophoresis on a gel (typically a denaturing and reducing protein gel, but may be any other type of gel including isoelectric focusing gels and the like). Following separation of proteins, the CA protein is detected by immunoblotting with antibodies raised against the CA protein. Methods of immunoblotting are well known to those of ordinary skill in the art.

In another preferred method, antibodies to the CA protein find use in in situ imaging techniques. In this method cells are contacted with from one to many antibodies to the CA protein(s). Following washing to remove non-specific antibody binding, the presence of the antibody or antibodies is detected. In one embodiment the antibody is detected by incubating with a secondary antibody that contains a detectable label. In another method the primary antibody to the CA protein(s) contains a detectable label. In another preferred embodiment each one of multiple primary antibodies contains a distinct and detectable label. This method finds particular use in simultaneous screening for a plurality of CA proteins. As will be appreciated by one of ordinary skill in the art, numerous other histological imaging techniques are useful in the invention.

In a preferred embodiment the label is detected in a fluorometer that has the ability to detect and distinguish emissions of different wavelengths. In addition, a fluorescence activated cell sorter (FACS) can be used in the method.

In another preferred embodiment, antibodies find use in diagnosing cancers from blood samples. As previously described, certain CA proteins are secreted/circulating molecules. Blood samples, therefore, are useful as samples to be probed or tested for the presence of secreted CA proteins. Antibodies can be used to detect the CA proteins by any of the previously described immunoassay techniques including ELISA, immunoblotting (Western blotting), immunoprecipitation, BIACORE technology and the like, as will be appreciated by one of ordinary skill in the art.

In a preferred embodiment, in situ hybridization of labeled CA nucleic acid probes to tissue arrays is done. For example, arrays of tissue samples, including CA tissue and/or normal tissue, are made. In situ hybridization as is known in the art can then be done.

It is understood that when comparing the expression fingerprints between an individual and a standard, the skilled artisan can make a diagnosis as well as a prognosis. It is further understood that the genes that indicate diagnosis may differ from those that indicate prognosis.

In a preferred embodiment, the CA proteins, antibodies, nucleic acids, modified proteins and cells containing CA sequences are used in prognosis assays. As above, gene expression profiles can be generated that correlate to cancer, especially lymphoma, severity, in terms of long term prognosis. Again, this may be done on either a protein or gene level, with the use of genes being preferred. As above, the CA probes are attached to biochips for the detection and quantification of CA sequences in a tissue or patient. The assays proceed as outlined for diagnosis.

Screening for CA-Targeted Drugs

In one embodiment, any of the CA sequences as described herein are used in drug screening assays. The CA proteins, antibodies, nucleic acids, modified proteins and cells containing CA sequences are used in drug screening assays or by evaluating the effect of drug candidates on a “gene expression profile” or expression profile of polypeptides. In one embodiment, the expression profiles are used, preferably in conjunction with high throughput screening techniques to allow monitoring for expression profile genes after treatment with a candidate agent, Zlokarnik, et al., Science 279, 84-8 (1998), Heid, et al., Genome Res., 6:986-994 (1996).

In another embodiment, the CA proteins, antibodies, nucleic acids, modified proteins and cells containing the native or modified CA proteins are used in screening assays. That is, the present invention provides novel methods for screening for compositions that modulate the cancer phenotype. As above, this can be done by screening for modulators of gene expression or for modulators of protein activity. Similarly, this may be done on an individual gene or protein level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the CA genes herein, a variety of assays to evaluate the effects of agents on gene expression may be executed. In a preferred embodiment, assays may be run on an individual gene or protein level. That is, having identified a particular gene as aberrantly regulated in cancer, candidate bioactive agents may be screened to modulate the gene's regulation. “Modulation” thus includes both an increase and a decrease in gene expression or activity. The preferred amount of modulation will depend on the original change of the gene expression in normal versus tumor tissue, with changes of at least 10%, preferably 50%, more preferably 100-300%, and in some embodiments 300-1000% or greater. Thus, if a gene exhibits a 4 fold increase in tumor compared to normal tissue, a decrease of about four fold is desired; a 10 fold decrease in tumor compared to normal tissue gives a 10 fold increase in expression for a candidate agent is desired, etc. Alternatively, where the CA sequence has been altered but shows the same expression profile or an altered expression profile, the protein will be detected as outlined herein.

As will be appreciated by those in the art, this may be done by evaluation at either the gene or the protein level; that is, the amount of gene expression may be monitored using nucleic acid probes and the quantification of gene expression levels, or, alternatively, the level of the gene product itself can be monitored, for example through the use of antibodies to the CA protein and standard immunoassays. Alternatively, binding and bioactivity assays with the protein may be done as outlined below.

In a preferred embodiment, gene expression monitoring is done and a number of genes, i.e. an expression profile, is monitored simultaneously, although multiple protein expression monitoring can be done as well.

In this embodiment, the CA nucleic acid probes are attached to biochips as outlined herein for the detection and quantification of CA sequences in a particular cell. The assays are further described below.

Generally, in a preferred embodiment, a candidate bioactive agent is added to the cells prior to analysis. Moreover, screens are provided to identify a candidate bioactive agent that modulates a particular type of cancer, modulates CA proteins, binds to a CA protein, or interferes between the binding of a CA protein and an antibody.

The term “candidate bioactive agent” or “drug candidate” or grammatical equivalents as used herein describes any molecule, e.g., protein, oligopeptide, small organic or inorganic molecule, polysaccharide, polynucleotide, etc., to be tested for bioactive agents that are capable of directly or indirectly altering either the cancer phenotype, binding to and/or modulating the bioactivity of a CA protein, or the expression of a CA sequence, including both nucleic acid sequences and protein sequences. In a particularly preferred embodiment, the candidate agent suppresses a CA phenotype, for example to a normal tissue fingerprint. Similarly, the candidate agent preferably suppresses a severe CA phenotype. Generally a plurality of assay mixtures are run in parallel with different agent concentrations to obtain a differential response to the various concentrations. Typically, one of these concentrations serves as a negative control, i.e., at zero concentration or below the level of detection.

In one aspect, a candidate agent will neutralize the effect of a CA protein. By “neutralize” is meant that activity of a protein is either inhibited or counter acted against so as to have substantially no effect on a cell.

Candidate agents encompass numerous chemical classes, though typically they are organic or inorganic molecules, preferably small organic compounds having a molecular weight of more than 100 and less than about 2,500 Daltons. Preferred small molecules are less than 2000, or less than 1500 or less than 11000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, or amidification to produce structural analogs.

In one embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and norleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of prokaryotic and eukaryotic proteins may be made for screening in the methods of the invention. Particularly preferred in this embodiment are libraries of bacterial, fungal, viral, and mammalian proteins, with the latter being preferred, and human proteins being especially preferred.

In another preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In one embodiment, the candidate bioactive agents are nucleic acids. As described generally for proteins, nucleic acid candidate bioactive agents may be naturally occurring nucleic acids, random nucleic acids, or “biased” random nucleic acids. In another embodiment, the candidate bioactive agents are organic chemical moieties, a wide variety of which are available in the literature.

In assays for testing alteration of the expression profile of one or more CA genes, after the candidate agent has been added and the cells allowed to incubate for some period of time, a nucleic acid sample containing the target sequences to be analyzed is prepared. The target sequence is prepared using known techniques (e.g., converted from RNA to labeled cDNA, as described above) and added to a suitable microarray. For example, an in vitro reverse transcription with labels covalently attached to the nucleosides is performed. Generally, the nucleic acids are labeled with a label as defined herein, especially with biotin-FITC or PE, Cy3 and Cy5.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions that allow formation of the label probe hybridization complex only in the presence of target. Stringency can be controlled by altering a step parameter that is a thermodynamic variable, including, but not limited to, temperature, formamide concentration, salt concentration, chaotropic salt concentrations pH, organic solvent concentration, etc. These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents in the assays. These include reagents like salts, buffers, neutral proteins, e.g. albumin, detergents, etc which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target. In addition, either solid phase or solution based (i.e., kinetic PCR) assays may be used.

Once the assay is run, the data are analyzed to determine the expression levels, and changes in expression levels as between states, of individual genes, forming a gene expression profile.

In a preferred embodiment, as for the diagnosis and prognosis applications, having identified the differentially expressed gene(s) or mutated gene(s) important in any one state, screens can be run to test for alteration of the expression of the CA genes individually. That is, screening for modulation of regulation of expression of a single gene can be done. Thus, for example, in the case of target genes whose presence or absence is unique between two states, screening is done for modulators of the target gene expression.

In addition, screens can be done for novel genes that are induced in response to a candidate agent. After identifying a candidate agent based upon its ability to suppress a CA expression pattern leading to a normal expression pattern, or modulate a single CA gene expression profile so as to mimic the expression of the gene from normal tissue, a screen as described above can be performed to identify genes that are specifically modulated in response to the agent. Comparing expression profiles between normal tissue and agent treated CA tissue reveals genes that are not expressed in normal tissue or CA tissue, but are expressed in agent treated tissue. These agent specific sequences can be identified and used by any of the methods described herein for CA genes or proteins. In particular these sequences and the proteins they encode find use in marking or identifying agent-treated cells. In addition, antibodies can be raised against the agent-induced proteins and used to target novel therapeutics to the treated CA tissue sample.

Thus, in one embodiment, a candidate agent is administered to a population of CA cells, that thus has an associated CA expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e. a peptide) may be put into a viral construct such as a retroviral construct and added to the cell, such that expression of the peptide agent is accomplished; see PCT US97/01019, hereby expressly incorporated by reference.

Once the candidate agent has been administered to the cells, the cells can be washed if desired and are allowed to incubate under preferably physiological conditions for some period of time. The cells are then harvested and a new gene expression profile is generated, as outlined herein.

Thus, for example, CA tissue may be screened for agents that reduce or suppress the CA phenotype. A change in at least one gene of the expression profile indicates that the agent has an effect on CA activity. By defining such a signature for the CA phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual genes and gene products (proteins). That is, having identified a particular differentially expressed gene as important in a particular state, screening of modulators of either the expression of the gene or the gene product itself can be done. The gene products of differentially expressed genes are sometimes referred to herein as “CA proteins” or “CAP”. The CAP may be a fragment, or alternatively, be the full-length protein to the fragment encoded by the nucleic acids of Tables 1-6. In a preferred embodiment, the CAP is selected from the human protein sequences shown in Tables 1-6 embodiment, the sequences are sequence variants as further described herein.

Preferably, the CAP is a fragment approximately 14 to 24 amino acids in length. More preferably the fragment is a soluble fragment. Preferably, the fragment includes a non-transmembrane region. In a preferred embodiment, the fragment has an N-terminal Cys to aid in solubility. In one embodiment, the C-terminus of the fragment is kept as a free acid and the N-terminus is a free amine to aid in coupling, e.g., to a cysteine.

In one embodiment the CA proteins are conjugated to an immunogenic agent as discussed herein. In one embodiment the CA protein is conjugated to BSA.

In a preferred embodiment, screening is done to alter the biological function of the expression product of the CA gene. Again, having identified the importance of a gene, in a particular state, screening for agents that bind and/or modulate the biological activity of the gene product can be run as is more fully outlined below.

In a preferred embodiment, screens are designed to first find candidate agents that can bind to CA proteins, and then these agents may be used in assays that evaluate the ability of the candidate agent to modulate the CAP activity and the cancer phenotype. Thus, as will be appreciated by those in the art, there are a number of different assays that may be run; binding assays and activity assays.

In a preferred embodiment, binding assays are done. In general, purified or isolated gene product is used; that is, the gene products of one or more CA nucleic acids are made. In general, this is done as is known in the art. For example, antibodies are generated to the protein gene products, and standard immunoassays are run to determine the amount of protein present. Alternatively, cells comprising the CA proteins can be used in the assays.

Thus, in a preferred embodiment, the methods comprise combining a CA protein and a candidate bioactive agent, and determining the binding of the candidate agent to the CA protein. Preferred embodiments utilize the human or mouse CA protein, although other mammalian proteins may also be used, for example for the development of animal models of human disease. In some embodiments, as outlined herein, variant or derivative CA proteins may be used.

Generally, in a preferred embodiment of the methods herein, the CA protein or the candidate agent is non-diffusably bound to an insoluble support having isolated sample receiving areas (e.g. a microtiter plate, an array, etc.). The insoluble support may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon®, etc. Microtiter plates and arrays are especially convenient because a large number of assays can be carried out simultaneously, using small amounts of reagents and samples.

The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the CA protein is bound to the support, and a candidate bioactive agent is added to the assay. Alternatively, the candidate agent is bound to the support and the CA protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate bioactive agent to the CA protein may be done in a number of ways. In a preferred embodiment, the candidate bioactive agent is labeled, and binding determined directly. For example, this may be done by attaching all or a portion of the CA protein to a solid support, adding a labeled candidate agent (for example a fluorescent label), washing off excess reagent, and determining whether the label is present on the solid support. Various blocking and washing steps may be utilized as is known in the art.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the proteins (or proteinaceous candidate agents) may be labeled at tyrosine positions using ¹²⁵I, or with fluorophores. Alternatively, more than one component may be labeled with different labels; using ¹²⁵I for the proteins, for example, and a fluorophore for the candidate agents.

In a preferred embodiment, the binding of the candidate bioactive agent is determined through the use of competitive binding assays. In this embodiment, the competitor is a binding moiety known to bind to the target molecule (i.e. CA protein), such as an antibody, peptide, binding partner, ligand, etc. Under certain circumstances, there may be competitive binding as between the bioactive agent and the binding moiety, with the binding moiety displacing the bioactive agent.

In one embodiment, the candidate bioactive agent is labeled. Either the candidate bioactive agent, or the competitor, or both, is added first to the protein for a time sufficient to allow binding, if present. Incubations may be performed at any temperature which facilitates optimal activity, typically between 4 and 40° C. Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate bioactive agent. Displacement of the competitor is an indication that the candidate bioactive agent is binding to the CA protein and thus is capable of binding to, and potentially modulating, the activity of the CA protein. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In an alternative embodiment, the candidate bioactive agent is added first, with incubation and washing, followed by the competitor. The absence of binding by the competitor may indicate that the bioactive agent is bound to the CA protein with a higher affinity. Thus, if the candidate bioactive agent is labeled, the presence of the label on the support, coupled with a lack of competitor binding, may indicate that the candidate agent is capable of binding to the CA protein.

In a preferred embodiment, the methods comprise differential screening to identity bioactive agents that are capable of modulating the activity of the CA proteins. In this embodiment, the methods comprise combining a CA protein and a competitor in a first sample. A second sample comprises a candidate bioactive agent, a CA protein and a competitor. The binding of the competitor is determined for both samples, and a change, or difference in binding between the two samples indicates the presence of an agent capable of binding to the CA protein and potentially modulating its activity. That is, if the binding of the competitor is different in the second sample relative to the first sample, the agent is capable of binding to the CA protein.

Alternatively, a preferred embodiment utilizes differential screening to identify drug candidates that bind to the native CA protein, but cannot bind to modified CA proteins. The structure of the CA protein may be modeled, and used in rational drug design to synthesize agents that interact with that site. Drug candidates that affect CA bioactivity are also identified by screening drugs for the ability to either enhance or reduce the activity of the protein.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Screening for agents that modulate the activity of CA proteins may also be done. In a preferred embodiment, methods for screening for a bioactive agent capable of modulating the activity of CA proteins comprise the steps of adding a candidate bioactive agent to a sample of CA proteins, as above, and determining an alteration in the biological activity of CA proteins. “Modulating the activity of a CA protein” includes an increase in activity, a decrease in activity, or a change in the type or kind of activity present. Thus, in this embodiment, the candidate agent should both bind to CA proteins (although this may not be necessary), and alter its biological or biochemical activity as defined herein. The methods include both in vitro screening methods, as are generally outlined above, and in vivo screening of cells for alterations in the presence, distribution, activity or amount of CA proteins.

Thus, in this embodiment, the methods comprise combining a CA sample and a candidate bioactive agent, and evaluating the effect on CA activity. By “CA activity” or grammatical equivalents herein is meant one of the CA protein's biological activities, including, but not limited to, its role in tumorigenesis, including cell division, preferably in lymphatic tissue, cell proliferation, tumor growth and transformation of cells. In one embodiment, CA activity includes activation of or by a protein encoded by a nucleic acid of Tables 1-6. An inhibitor of CA activity is the inhibition of any one or more CA activities.

In a preferred embodiment, the activity of the CA protein is increased; in another preferred embodiment, the activity of the CA protein is decreased. Thus, bioactive agents that are antagonists are preferred in some embodiments, and bioactive agents that are agonists may be preferred in other embodiments.

In a preferred embodiment, the invention provides methods for screening for bioactive agents capable of modulating the activity of a CA protein. The methods comprise adding a candidate bioactive agent, as defined above, to a cell comprising CA proteins. Preferred cell types include almost any cell. The cells contain a recombinant nucleic acid that encodes a CA protein. In a preferred embodiment, a library of candidate agents is tested on a plurality of cells.

In one aspect, the assays are evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, bioactive agents are identified. Compounds with pharmacological activity are able to enhance or interfere with the activity of the CA protein.

Applications of the Invention

In one embodiment, a method of inhibiting cancer cell division is provided. In another embodiment, a method of inhibiting tumor growth is provided. In a further embodiment, methods of treating cells or individuals with cancer are provided.

In one embodiment, a method of inhibiting carcinoma cancer cell division, is provided. The method comprises administration of a carcinoma cancer inhibitor. In oned embodiment, the carcinoma cell is a lymphoma carcinoma, in another embodiment, the carcinoma cell is a breast camcer carcinoma.

In another embodiment, a method of inhibiting tumor growth is provided. The method comprises administration of a carcinoma cancer inhibitor. In a particular embodiment, a method of inhibiting tumor growth in lymphatic tissue is provided comprising administration of a lymphoma inhibitor. In another embodiment, a method of inhibiting tumor growth in mammary tissue is provided comprising administration of a breast cancer inhibitor.

The method comprises administration of a cancer inhibitor. In particular embodiments, the cancer inhibitor is an antisense molecule, a pharmaceutical composition, a therapeutic agent or small molecule, or a monoclonal, polyclonal, chimeric or humanized antibody. In particular embodiments, a therapeutic agent is coupled with a an antibody, preferable a monoclonal antobody.

In other embodiments, methods for detection or diagnosis of cancer cells in an individual are provided. In particular embodiments, the diagnostic/detection agent is a small molecule that pereferentially binds to a CAP according to the invention. In one embodiment, the diagnostic/detection agent is an antibody, preferably a monoclonal antibody, preferably linked to a detectable agent.

In other embodiments of the invention, animal models and transgenic animals are provided, which find use in generating animal models of cancers, particularly lymphomas and carcinomas.

(a) Antisense Molecules

In one embodiment, the cancer inhibitor is an antisense molecule. Antisense molecules as used herein include antisense or sense oligonucleotides comprising a single-stranded nucleic acid sequence (either RNA or DNA) capable of binding to target mRNA (sense) or DNA (antisense) sequences for cancer molecules. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen, Cancer Res. 48:2659, (1988) and van der Krol et al., BioTechniques 6:958, (1988).

Antisense molecules may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide may be introduced into a cell containing the target nucleic acid sequence by formation of an oligonucleotide-lipid complex, as described in WO 90/10448. It is understood that the use of antisense molecules or knock out and knock in models may also be used in screening assays as discussed above, in addition to methods of treatment.

(b) Pharmaceutical Compositions

Pharmaceutical compositions encompassed by the present invention include as active agent, the polypeptides, polynucleotides, antisense oligonucleotides, or antibodies of the invention disclosed herein in a therapeutically effective amount. An “effective amount” is an amount sufficient to effect beneficial or desired results, including clinical results. An effective amount can be administered in one or more administrations. For purposes of this invention, an effective amount of an adenoviral vector is an amount that is sufficient to palliate, ameliorate, stabilize, reverse, slow or delay the progression of the disease state.

The compositions can be used to treat cancer as well as metastases of primary cancer. In addition, the pharmaceutical compositions can be used in conjunction with conventional methods of cancer treatment, e.g., to sensitize tumors to radiation or conventional chemotherapy. The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

Where the pharmaceutical composition comprises an antibody that specifically binds to a gene product encoded by a differentially expressed polynucleotide, the antibody can be coupled to a drug for delivery to a treatment site or coupled to a detectable label to facilitate imaging of a site comprising cancer cells, such as prostate cancer cells. Methods for coupling antibodies to drugs and detectable labels are well known in the art, as are methods for imaging using detectable labels.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. The effective amount for a given situation is determined by routine experimentation and is within the judgment of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg of the compositions of the present invention in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

The pharmaceutical compositions of the present invention comprise a CA protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

The compounds having the desired pharmacological activity may be administered in a physiologically acceptable carrier to a host, as previously described. The agents may be administered in a variety of ways, orally, parenterally e.g., subcutaneously, intraperitoneally, intravascularly, etc. Depending upon the manner of introduction, the compounds may be formulated in a variety of ways. The concentration of therapeutically active compound in the formulation may vary from about 0.1-100% wgt/vol. Once formulated, the compositions contemplated by the invention can be (1) administered directly to the subject (e.g., as polynucleotide, polypeptides, small molecule agonists or antagonists, and the like); or (2) delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, intratumoral or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Once differential expression of a gene corresponding to a CA polynucleotide described herein has been found to correlate with a proliferative disorder, such as neoplasia, dysplasia, and hyperplasia, the disorder can be amenable to treatment by administration of a therapeutic agent based on the provided polynucleotide, corresponding polypeptide or other corresponding molecule (e.g., antisense, ribozyme, etc.). In other embodiments, the disorder can be amenable to treatment by administration of a small molecule drug that, for example, serves as an inhibitor (antagonist) of the function of the encoded gene product of a gene having increased expression in cancerous cells relative to normal cells or as an agonist for gene products that are decreased in expression in cancerous cells (e.g., to promote the activity of gene products that act as tumor suppressors).

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic compositions agents includes local or systemic administration, including injection, oral administration, particle gun or catheterized administration, and topical administration. Preferably, the therapeutic polynucleotide composition contains an expression construct comprising a promoter operably linked to a polynucleotide of at least 12, 22, 25, 30, or 35 contiguous nt of the polynucleotide disclosed herein. Various methods can be used to administer the therapeutic composition directly to a specific site in the body. For example, a small metastatic lesion is located and the therapeutic composition injected several times in several different locations within the body of tumor. Alternatively, arteries that serve a tumor are identified, and the therapeutic composition injected into such an artery, in order to deliver the composition directly into the tumor. A tumor that has a necrotic center is aspirated and the composition injected directly into the now empty center of the tumor. An antisense composition is directly administered to the surface of the tumor, for example, by topical application of the composition. X-ray imaging is used to assist in certain of the above delivery methods.

Targeted delivery of therapeutic compositions containing an antisense polynucleotide, subgenomic polynucleotides, or antibodies to specific tissues can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g., for enhancing or inhibiting levels of the encoded gene product) and efficacy of transformation and expression are considerations that will affect the dosage required for ultimate efficacy of the antisense subgenomic polynucleotides. Where greater expression is desired over a larger area of tissue, larger amounts of antisense subgenomic polynucleotides or the same amounts re-administered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell Biol. (1994) 14:2411, and in Woffendin, Proc. Natl. Acad. Sci. (1994) 91:1581.

Further non-viral delivery suitable for use includes mechanical delivery systems such as the approach described in Woffendin et al., Proc. Natl. Acad. Sci. USA (1994) 91(24):11581. Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

The administration of the CA proteins and modulators of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the CA proteins and modulators may be directly applied as a solution or spray.

In a preferred embodiment, CA proteins and modulators are administered as therapeutic agents, and can be formulated as outlined above. Similarly, CA genes (including both the full-length sequence, partial sequences, or regulatory sequences of the CA coding regions) can be administered in gene therapy applications, as is known in the art. These CA genes can include antisense applications, either as gene therapy (i.e. for incorporation into the genome) or as antisense compositions, as will be appreciated by those in the art.

Thus, in one embodiment, methods of modulating CA gene activity in cells or organisms are provided. In one embodiment, the methods comprise administering to a cell an anti-CA antibody that reduces or eliminates the biological activity of an endogenous CA protein. Alternatively, the methods comprise administering to a cell or organism a recombinant nucleic acid encoding a CA protein. As will be appreciated by those in the art, this may be accomplished in any number of ways. In a preferred embodiment, for example when the CA sequence is down-regulated in cancer, the activity of the CA gene product is increased by increasing the amount of CA expression in the cell, for example by overexpressing the endogenous CA gene or by administering a gene encoding the CA sequence, using known gene-therapy techniques. In a preferred embodiment, the gene therapy techniques include the incorporation of the exogenous gene using enhanced homologous recombination (EHR), for example as described in PCT/US93/03868, hereby incorporated by reference in its entirety. Alternatively, for example when the CA sequence is up-regulated in cancer, the activity of the endogenous CA gene is decreased, for example by the administration of a CA antisense nucleic acid.

(c) Vaccines

In a preferred embodiment, CA genes are administered as DNA vaccines, either single genes or combinations of CA genes. Naked DNA vaccines are generally known in the art. Brower, Nature Biotechnology, 16:1304-1305 (1998).

In one embodiment, CA genes of the present invention are used as DNA vaccines. Methods for the use of genes as DNA vaccines are well known to one of ordinary skill in the art, and include placing a CA gene or portion of a CA gene under the control of a promoter for expression in a patient with cancer. The CA gene used for DNA vaccines can encode full-length CA proteins, but more preferably encodes portions of the CA proteins including peptides derived from the CA protein. In a preferred embodiment a patient is immunized with a DNA vaccine comprising a plurality of nucleotide sequences derived from a CA gene. Similarly, it is possible to immunize a patient with a plurality of CA genes or portions thereof. Without being bound by theory, expression of the polypeptide encoded by the DNA vaccine, cytotoxic T-cells, helper T-cells and antibodies are induced that recognize and destroy or eliminate cells expressing CA proteins.

In a preferred embodiment, the DNA vaccines include a gene encoding an adjuvant molecule with the DNA vaccine. Such adjuvant molecules include cytokines that increase the immunogenic response to the CA polypeptide encoded by the DNA vaccine. Additional or alternative adjuvants are known to those of ordinary skill in the art and find use in the invention.

(d) Antibodies

In one embodiment, a cancer inhibitor is an antibody as discussed above. In one embodiment, the CA-proteins of the present invention may be used to generate polyclonal and monoclonal antibodies to CA proteins, which are useful as described herein. Similarly, the CA proteins can be coupled, using standard technology, to affinity chromatography columns. These columns may then be used to purify CA antibodies. In a preferred embodiment, the antibodies are generated to epitopes unique to a CA protein; that is, the antibodies show little or no cross-reactivity to other proteins. These antibodies find use in a number of applications. For example, the CA antibodies may be coupled to standard affinity chromatography columns and used to purify CA proteins. The antibodies may also be used therapeutically as blocking polypeptides, as outlined above, since they will specifically bind to the CA protein.

The present invention further provides methods for detecting the presence of and/or measuring a level of a polypeptide in a biological sample, which CA polypeptide is encoded by a CA polynucleotide that is differentially expressed in a cancer cell, using an antibody specific for the encoded polypeptide. The methods generally comprise: a) contacting the sample with an antibody specific for a polypeptide encoded by a CA polynucleotide that is differentially expressed in a prostate cancer cell; and b) detecting binding between the antibody and molecules of the sample.

Detection of specific binding of the antibody specific for the encoded cancer-associated polypeptide, when compared to a suitable control is an indication that encoded polypeptide is present in the sample. Suitable controls include a sample known not to contain the encoded CA polypeptide or known not to contain elevated levels of the polypeptide; such as normal tissue, and a sample contacted with an antibody not specific for the encoded polypeptide, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay. In general, the specific antibody will be detectably labeled, either directly or indirectly. Direct labels include radioisotopes; enzymes whose products are detectable (e.g., luciferase, β-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (green fluorescent protein), and the like. The antibody may be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The biological sample may be brought into contact with and immobilized on a solid support or carrier, such as nitrocellulose, that is capable of immobilizing cells, cell particles, or soluble proteins. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls, and to appropriate standards.

In some embodiments, the methods are adapted for use in vivo, e.g., to locate or identify sites where cancer cells are present. In these embodiments, a detectably-labeled moiety, e.g., an antibody, which is specific for a cancer-associated polypeptide is administered to an individual (e.g., by injection), and labeled cells are located using standard imaging techniques, including, but not limited to, magnetic resonance imaging, computed tomography scanning, and the like. In this manner, cancer cells are differentially labeled.

(e) Detection and Diagnosis of Cancers

Without being bound by theory, it appears that the various CA sequences are important in cancers. Accordingly, disorders based on mutant or variant CA genes may be determined. In one embodiment, the invention provides methods for identifying cells containing variant CA genes comprising determining all or part of the sequence of at least one endogenous CA genes in a cell. As will be appreciated by those in the art, this may be done using any number of sequencing techniques. In a preferred embodiment, the invention provides methods of identifying the CA genotype of an individual comprising determining all or part of the sequence of at least one CA gene of the individual. This is generally done in at least one tissue of the individual, and may include the evaluation of a number of tissues or different samples of the same tissue. The method may include comparing the sequence of the sequenced CA gene to a known CA gene, i.e., a wild-type gene. As will be appreciated by those in the art, alterations in the sequence of some CA genes can be an indication of either the presence of the disease, or propensity to develop the disease, or prognosis evaluations.

The sequence of all or part of the CA gene can then be compared to the sequence of a known CA gene to determine if any differences exist. This can be done using any number of known homology programs, such as Bestfit, etc. In a preferred embodiment, the presence of a difference in the sequence between the CA gene of the patient and the known CA gene is indicative of a disease state or a propensity for a disease state, as outlined herein.

In a preferred embodiment, the CA genes are used as probes to determine the number, of copies of the CA gene in the genome. For example, some cancers exhibit chromosomal deletions or insertions, resulting in an alteration in the copy number of a gene.

In another preferred embodiment CA genes are used as probes to determine the chromosomal location of the CA genes. Information such as chromosomal location finds use in providing a diagnosis or prognosis in particular when chromosomal abnormalities such as translocations, and the like are identified in CA gene loci.

The present invention provides methods of using the polynucleotides described herein for detecting cancer cells, facilitating diagnosis of cancer and the severity of a cancer (e.g., tumor grade, tumor burden, and the like) in a subject, facilitating a determination of the prognosis of a subject, and assessing the responsiveness of the subject to therapy (e.g., by providing a measure of therapeutic effect through, for example, assessing tumor burden during or following a chemotherapeutic regimen). Detection can be based on detection of a polynucleotide that is differentially expressed in a cancer cell, and/or detection of a polypeptide encoded by a polynucleotide that is differentially expressed in a cancer cell. The detection methods of the invention can be conducted in vitro or in vivo, on isolated cells, or in whole tissues or a bodily fluid e.g., blood, plasma, serum, urine, and the like).

In some embodiments, methods are provided for detecting a cancer cell by detecting expression in the cell of a transcript that is differentially expressed in a cancer cell. Any of a variety of known methods can be used for detection, including, but not limited to, detection of a transcript by hybridization with a polynucleotide that hybridizes to a polynucleotide that is differentially expressed in a prostate cancer cell; detection of a transcript by a polymerase chain reaction using specific oligonucleotide primers; in situ hybridization of a cell using as a probe a polynucleotide that hybridizes to a gene that is differentially expressed in a prostate cancer cell. The methods can be used to detect and/or measure mRNA levels of a gene that is differentially expressed in a cancer cell. In some embodiments, the methods comprise: a) contacting a sample with a polynucleotide that corresponds to a differentially expressed gene described herein under conditions that allow hybridization; and b) detecting hybridization, if any.

Detection of differential hybridization, when compared to a suitable control, is an indication of the presence in the sample of a polynucleotide that is differentially expressed in a cancer cell. Appropriate controls include, for example, a sample that is known not to contain a polynucleotide that is differentially expressed in a cancer cell, and use of a labeled polynucleotide of the same “sense” as the polynucleotide that is differentially expressed in the cancer cell. Conditions that allow hybridization are known in the art, and have been described in more detail above. Detection can also be accomplished by any known method, including, but not limited to, in situ hybridization, PCR (polymerase chain reaction), RT-PCR (reverse transcription-PCR), TMA, bDNA, and Nasbau and “Northern” or RNA blotting, or combinations of such techniques, using a suitably labeled polynucleotide. A variety of labels and labeling methods for polynucleotides are known in the art and can be used in the assay methods of the invention. Specificity of hybridization can be determined by comparison to appropriate controls.

Polynucleotides generally comprising at least 10 nt, at least 12 nt or at least 15 contiguous nucleotides of a polynucleotide provided herein, such as, for example, those having the sequence as depicted in Tables 1-6, are used for a variety of purposes, such as probes for detection of and/or measurement of, transcription levels of a polynucleotide that is differentially expressed in a prostate cancer cell. As will be readily appreciated by the ordinarily skilled artisan, the probe can be detectably labeled and contacted with, for example, an array comprising immobilized polynucleotides obtained from a test sample (e.g., mRNA). Alternatively, the probe can be immobilized on an array and the test sample detectably labeled. These and other variations of the methods of the invention are well within the skill in the art and are within the scope of the invention.

Nucleotide probes are used to detect expression of a gene corresponding to the provided polynucleotide. In Northern blots, mRNA is separated electrophoretically and contacted with a probe. A probe is detected as hybridizing to an mRNA species of a particular size. The amount of hybridization can be quantitated to determine relative amounts of expression, for example under a particular condition. Probes are used for in situ hybridization to cells to detect expression. Probes can also be used in vivo for diagnostic detection of hybridizing sequences. Probes are typically labeled with a radioactive isotope. Other types of detectable labels can be used such as chromophores, fluorophores, and enzymes. Other examples of nucleotide hybridization assays are described in WO92/02526 and U.S. Pat. No. 5,124,246.

PCR is another means for detecting small amounts of target nucleic acids (see, e.g., Mullis et al., Meth. Enzymol. (1987) 155:335; U.S. Pat. No. 4,683,195; and U.S. Pat. No. 4,683,202). Two primer oligonucleotides that hybridize with the target nucleic acids are used to prime the reaction. The primers can be composed of sequence within or 3′ and 5′ to the CA polynucleotides disclosed herein. Alternatively, if the primers are 3′ and 5′ to these polynucleotides, they need not hybridize to them or the complements. After amplification of the target with a thermostable polymerase, the amplified target nucleic acids can be detected by methods known in the art, e.g., Southern blot. mRNA or cDNA can also be detected by traditional blotting techniques (e.g., Southern blot, Northern blot, etc.) described in Sambrook et al., “Molecular Cloning: A Laboratory Manual” (New York, Cold Spring Harbor Laboratory, 1989) (e.g., without PCR amplification). In general, mRNA or cDNA generated from mRNA using a polymerase, enzyme can be purified and separated using gel electrophoresis, and transferred to a solid support, such as nitrocellulose. The solid support is exposed to a labeled probe, washed to remove any unhybridized probe, and duplexes containing the labeled probe are detected.

Methods using PCR amplification can be performed on the DNA from a single cell, although it is convenient to use at least about 10⁵ cells. The use of the polymerase chain reaction is described in Saiki et al. (1985) Science 239:487, and a review of current techniques may be found in Sambrook, et al. Molecular Cloning: A Laboratory Manual, CSH Press 1989, pp.14.2-14.33. A detectable label may be included in the amplification reaction. Suitable detectable labels include fluorochromes,(e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)), radioactive labels, (e.g. ³²P, ³⁵S, ³H, etc.), and the like. The label may be a two stage system, where the polynucleotides is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The detection methods can be provided as part of a kit. Thus, the invention further provides kits for detecting the presence and/or a level of a polynucleotide that is differentially expressed in a cancer cell (e.g., by detection of an mRNA encoded by the differentially expressed gene of interest), and/or a polypeptide encoded thereby, in a biological sample. Procedures using these kits can be performed by clinical laboratories, experimental laboratories, medical practitioners, or private individuals. The kits of the invention for detecting a polypeptide encoded by a polynucleotide that is differentially expressed in a cancer cell may comprise a moiety that specifically binds the polypeptide, which may be an antibody that binds the polypeptide or fragment thereof. The kits of the invention used for detecting a polynucleotide that is differentially expressed in a prostate cancer cell may comprise a moiety that specifically hybridizes to such a polynucleotide. The kit may optionally provide additional components that are useful in the procedure, including, but not limited to, buffers, developing reagents, labels, reacting surfaces, means for detection, control samples, standards, instructions, and interpretive information. Accordingly, the present invention provides kits for detecting prostate cancer comprising at least one of polynucleotides having the sequence as shown in Tables 1-6 or fragments thereof.

The present invention further relates to methods of detecting/diagnosing a neoplastic or preneoplastic condition in a mammal (for example, a human). “Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and therametrics (e.g., monitoring a subject's condition to provide information as to the effect or efficacy of therapy).

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

An “effective amount” is an amount sufficient to effect beneficial or desired results, including clinical results. An effective amount can be administered in one or more administrations.

A “cell sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “cell sample” encompasses a clinical sample, and also includes cells in culture, cell supernatants, cell lysates, serum, plasma, biological fluid, and tissue samples.

As used herein, the terms “neoplastic cells”, “neoplasia”, “tumor”, “tumor cells”, “cancer” and “cancer cells”, (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign.

The terms “individual,” “subject,” “host,” and “patient,” are used interchangeably herein and refer to any mammalian subject for whom diagnosis, treatment, or therapy is desired, particularly humans. Other subjects may include cattle, dogs, cats, guinea pigs, rabbits, rats, mice, horses, and so on. Examples of conditions that can be detected/diagnosed in accordance with these methods include cancers. Polynucleotides corresponding to genes that exhibit the appropriate expression pattern can be used to detect cancer in a subject. For a review of markers of cancer, see, e.g., Hanahan et al. Cell 100:57-70 (2000).

One detection/diagnostic method comprises: (a) obtaining from a mammal (e.g., a human) a biological sample, (b) detecting the presence in the sample of a CA protein and (c) comparing the amount of product present with that in a control sample. In accordance with this method, the presence in the sample of elevated levels of a CA gene product indicates that the subject has a neoplastic or preneoplastic condition.

Biological samples suitable for use in this method include biological fluids such as serum, plasma, pleural effusions, urine and cerebro-spinal fluid, CSF, tissue samples (e.g., mammary tumor or prostate tissue slices) can also be used in the method of the invention, including samples derived from biopsies. Cell cultures or cell extracts derived, for example, from tissue biopsies can also be used.

The compound is preferably a binding protein, e.g., an antibody, polyclonal or monoclonal, or antigen binding fragment thereof, which can be labeled with a detectable marker (e.g., fluorophore, chromophore or isotope, etc). Where appropriate, the compound can be attached to a solid support such as a bead, plate, filter, resin, etc. Determination of formation of the complex can be effected by contacting the complex with a further compound (e.g., an antibody) that specifically binds to the first compound (or complex). Like the first compound, the further compound can be attached to a solid support and/or can be labeled with a detectable marker.

The identification of elevated levels of CA protein in accordance with the present invention makes possible the identification of subjects (patients) that are likely to benefit from adjuvant therapy. For example, a biological sample from a post primary therapy subject (e.g., subject having undergone surgery) can be screened for the presence of circulating CA protein, the presence of elevated levels of the protein, determined by studies of normal populations, being indicative of residual tumor tissue. Similarly, tissue from the cut site of a surgically removed tumor can be examined (e.g., by immunofluorescence), the presence of elevated levels of product (relative to the surrounding tissue) being indicative of incomplete removal of the tumor. The ability to identify such subjects makes it possible to tailor therapy to the needs of the particular subject. Subjects undergoing non-surgical therapy, e.g., chemotherapy or radiation therapy, can also be monitored, the presence in samples from such subjects of elevated levels of CA protein being indicative of the need for continued treatment. Staging of the disease (for example, for purposes of optimizing treatment regimens) can also be effected, for example, by biopsy e.g., with antibody specific for a CA protein.

(f) Animal Models and Transgenics

In another preferred embodiment CA genes find use in generating animal models of cancers, particularly lymphomas and carcinomas. As is appreciated by one of ordinary skill in the art, when the CA gene identified is repressed or diminished in CA tissue, gene therapy technology wherein antisense RNA directed to the CA gene will also diminish or repress expression of the gene. An animal generated as such serves as an animal model of CA that finds use in screening bioactive drug candidates. Similarly, gene knockout technology, for example as a result of homologous recombination with an appropriate gene targeting vector, will result in the absence of the CA protein. When desired, tissue-specific expression or knockout of the CA protein may be necessary.

It is also possible that the CA protein is overexpressed in cancer. As such, transgenic animals can be generated that overexpress the CA protein. Depending on the desired expression level, promoters of various strengths can be employed to express the transgene. Also, the number of copies of the integrated transgene can be determined and compared for a determination of the expression level of the transgene. Animals generated by such methods find use as animal models of CA and are additionally useful in screening for bioactive molecules to treat cancer.

(g) Characterization of CA Sequences

The CA nucleic acid sequences of the invention are depicted in Tables 1-6. The sequences in each Table include genomic DNA sequence, sequence corresponding to the mRNA and amino acid sequences of the proteins encoded by the mRNA for both mouse and human genes. The different sequences are assigned the following SEQ ID Nos:

TABLE 1
(mouse gene: Fscn1; human gene SNL)
Mouse genomic sequence (SEQ ID NO: 1)
Mouse mRNA sequence (SEQ ID NO: 2)
Mouse coding sequence (SEQ ID NO: 3)
Human genomic sequence (SEQ ID NO: 4)
Human mRNA sequence (SEQ ID NO: 5)
Human coding sequence (SEQ ID NO: 6)

| | |
|---|---|
| MOUSE SEQUENCE - GENOMIC | (SEQ ID NO: 1) |

CCTCTCAAACTCTTGGTAATTCTCCTGCTTCGGCACGCTGGGGACTGGGACTACACCTGTGTGCCACCATGGCTGGCTATTTCTCTCTC
TTGAACACTGGAAGAGTGCTCAGAGTTTACTGGATCTGGGAGAAGCTCGAGGCTGGGTTATGGAAGCCGGCTCTGCTTGCTCCAGCTCA
GGGAGGAGTTGCCTGAGGGCCAGGCACTTCGAAGGACAGATGGGACCCAAGGCCAGACACTGGGCCCCAGCTCACCAGAAGTGGAGCCC
TGACTGTCTCTGAGAGGGTAAGCTGGGGTGGCTGCCAGGCGGGCAAGGCCAAAGCCTGGCAGCAGCCGGTGGCCCCTCTTCTGGCAGAG
ATATCTTGGCATGAGCCTGGCTCTGCCCATGACACTAAAGTGCCCTCTTAATTAGCCAGGCTCTTGCCAAGCACTGGCCACGATTGCCT
TGTTTCACAGAATTCACCTTCGACTGGCACAGATGGGGAGGGTGGATAGCCCGGTGTTTTGTCTTTCTTCTAGGGGAGCTGGGCTCAAG
GGCAGGACTCCTGGGCCAGCCCAGGTTTTTCCCTCAGAAACCACAGCATGAATACTGGCATGATGGAGCACACCTGTAATCAGCAGAAA
TTGAGACAGGAAGATTGTTGAGAATTTGAGGCCAACCTAGGCTAAATAGGGAGACCCTATCTCTACACACTCCCCCTCCCCCCGCCCCT
GGAAATGCTGGAGGGTCTGGGGAAGATGGCTCAGTGGTTAAGGGCACTTGCTGCTCTTGCAGGGGACCCAAGTTTGATTCCCAGCATCT
ATAACTTGGTGGCTCACAATTCCAGTTCCAGGCACTCTGACACCTTCCTGTGGTCTGCAAGGCACCTGCATTCATTCTCACGTGCATGC
GCACACACATGCGCGCGCGCACACACACACACACACACACACACACATCTCATAAATACAAATAAAATAAATCTTGAAAACAACAGTGA
CAACAACACATGCTGAACATGGTGGTGCTCCTGACATGGTGGTGCTCCTGACATGGTGGTGCTCCTGACATGGTGATGCTCCTGACATG
GTGGTGCTCCTGACATGATGGTAAGCCTGACATGGTGGTGCTCCTGACATGGTGGTGCTCCTGACATGGTGATGCTCCTGACATGGTGG
TGCTCCTGACATGGTGGTAAGCCTGACATGGTGGTAAGCCTGACATGGTGGTGCTCCTGACATGGTGGTGCTCCCTGCACTGACAGTTC
TGAGGAGGTGGAGATAGGAGGATCCACACTTGGAAGCTTGCCTGAGATACACCCTGTCTCAGAATAGGCTCGAGGCTGCCTCTGCCCTT
TTGCTTCTATCTCTTTTTCCATATGTGTGTGTGCGTGTGTGCATATGTGTATGCATATTTGTAAGTATATGTATATAAATAAACACACA
CACACACAAACTTACTCTGTATACTAGGCTGGCCTTGAACTCAGAGACCTGCCTGTTTCTGCCTTCCCAGTGCTGGGATTAAAAGCGTG
TGCCATGGGCTGGTGAGATGGCTCAGTGGGTAAGAGTACCTGACTGCTCTTCCAAAGGTCCAGTGTTCAAGTCCCAGCAACCACATGGT
GGCTCACAACCATCCGTAACAAGATCTGACGCCCTCTTCTGGTGTGTCTGAAGACAGCTACAGTGTACTTAAATAAATAAATCTTAAAA
AAAAAAAAAAAAAAAAAAAAAGGCGTGCACCACCACTGCCCGGCTAGTTAGCTTTATCTTCCGTGGATGTTTTGTCTGCAGTATGTCTG
AGTGAGGGTGTCAGATCCTCTGTAACTGGAGTTATAGACAGTTGTGAGCAGCCATGTAAGTGCTGGGAATTGAATCCGGGTTCTCTGGA
AGAGCAGTTCATGCTCTTAAGCACTGCGCCATCTCTCCAGCTGTCCTTATTTATTTTTAAGGGTCTCTTGTAGCCATACAGGCCAGAAA
CCCACCATTAGCCACGGACCTTGAGCTCCAAGTGCTGGGTGACAGGCCCGCATCTCTCCTGGTTTGTGATCTGCTAGGGATGGGGTCTA
GGACCTCACACAGGCTGCATAAGCATTCCCCTCACCCACCCTCATCCCCACCCAGTCCTGGACAGGCTCTGGCTGTGTAGGCTGGTCTC
CTCCTTTCTATCCCCCTGCCTCAGCCTCCTGAGTGCTGAGGNNNNNNNNNNNNNNNNNNNNNNNNNNNNNNNNNNNNNNNNNNNNNNNN
NNNNNNNNNNNNNNNNNNNNNNNNNNNNNNNNNNNNNCTGTGGTGGCTCCCGAGTGCTGTCATCCCAGCACTGGGGAGGTTGAAGCAGG
AAGGAGAATCACAGGTTCAAGGCCAGCTTGGTTTACTTGAGACTCTGTCTCAAAAAGACCAAACCAGGGACTGGGAGGATGGCCAGTAA
AATGCTTTTCCAGCTATCGAGGGAACCTGAGTTTCATCCTGGAACTCATACAAAAAGCTGGCTGTGGTCAGGTGGCGCATGCCTTTAAT
CCCATCACTCAGGAGACAGAGGCAGGCAGATTCTTGAGTTCGAGGCCAGCCTGGTCTACAGACTGAGTTCCAGGATAGCCAGGTCTACA
CAGAGAAACCCTATCTTGGAAAACAAAACAAACAAAAAAAACTAGGTCTGGCTAGCCTGTAACCCCAGTGCTGGGTAAACAGGGGCAGG
TGAATACATCTCCGGGATTCGCCAGCCTGTCCTATCTATGAGCTCTAGGTCTGGGGAGACAGCCTGTCAAATCAATGCAAGCAGGTGAC
AACCGAGGAAAGACACCCAAGGTTGATGCACGGGCAAGTGTGCACACACACACACCACAGCAAACAAAAACTAACAAAACCCAAACCAT
ATTTTGCTGTGGTGGTGGTTGTTAAATTTTATTTATTCTGTGTGTATGGGTATTTCACCTGCATGAATATCTACCTGTGTAGCATATAC
ATGCCGGGTACTTTCGGAGGTCAGAAGAGGACATCAAATCCCGTAGAACTGGAGTTACAGAAAATGAACTTGTGGCTTCTGGGAATCTA
ACTTGGATCCTCTAAACGACAGGCAGGACTCTGAATTCTTGAGCCACAGATACAGCTTTCCAAACCCATGTTTTGTAACAGCCACCAGG
CCTGAAAACTCTGGGCCATGTCCCGTTTGTTGTGTCTAGGCCTCAGTTTCCTGGAAGGCAGAGAATGCGTAGGTCCGTTGTTAGTGGAG
ACAGCAGTGGTCGTAACAGCCTGGTCTACCCTCTTGGTCAGAGGGAGGCAGGCGGAGAGTAACTCAGGGCCTGGGGGCAACACCCAGCT
GGGCCTGGGGCTAGTGGGGGCTCAGCCAGGCAGGACGTGGGTCCTCCTACTTCCTAAGCTCTGATGGGCAAGTGGTGCCAGTGGTACCA
GGCTGCCCTGAGGGGCCCTTAGAGCAGGTGACCACAGAGCCACAAAGAGGCTATTCATGGCCTCCGGTTCACGAGGCTGCCCCTTTATT
GAGGGCTTGAACCACAGAAAGCTGTGTGGTCTGCAGGGAAGCTAGTTCTGAGCTGTCTGGCCAGCTACAGTAGTCTGTGTGTGTGTGTG
TGTGTGTATACACATGGTACTTTTTTCTATCTATCTATCTATCTATCTATCTATCTATCTATCTATCTATCTATCTATCTTTGTGTCTA
TGTATCTAATAATAAATTGATCTATTTAACTATGTAGATATCAATCCATTGATCTATGTATCTATCAACTCATGTCTATCTATGTATGG
ATGTACCTACATCCGTACTTGAGGCACGACTTAAAGTACCCCAGGCTAGCCTTACCACATTTCATAGCACAGGATGGTCTTGAACTTCA
GGTCCTCCTGTCTCTACCTCGTCACTGCTCCGATTACAGGTTTGTGCCACCACGCCTGGCTTACCAGCTTCTGTGGTTAGGTTCCAGGA
TTTCACTATCAACTGCGCCACATCGTCAGCCATATTCAATATCCTTTCTTTTACATTCTTACTTATTTACTACAAATCGGTCCCAGGAA
CCAAGGTCGCCAAGCCTGATCGTCAGTGCTCTTCCCTGCTCCCTGACTTATTTGCTCTTTTTTTTTTTTTTTTTTTTTTCTAGACAGGG
TTTCTCTCTGTAGCCCTGGCTGTCCTGGAACTCACTTTGTAGACCAGGCTGGCCTCGAACTCAGAAATCTGCCTGCCTCTGTCCCCAAC
CCCACCCCCAGTACTGGGATTAAAGGCATGCGCCACACGCCCAGCTCTTATTTGCTCCTTTGAATTAAGGTCTCATGTACACAAGGCTG
ACCGTTCATGCACAATACAGCTAAGCCTTCCTTTGAATTTCTCAGTTTCTTTCTTCTACCCAACAGTTTCTGAGATTTCAGGTCCGTCC
TATGTCATCCCCAGGTTTTTTGTTTGTTTCTTTGTTTGTTTTTTGGTTTTGTTTTGCTGTTTTTCAACACAGAGTTTCTCTGTGGAACC
CTGGCTATCCTGGAACTCTGTATACCAGGCTGTCCTTGAACTCACAGACCCTCTCCCTCCCTAGTGCTGCGTTAAAGGTCCCTGCCATC
ACCGTGAAACTTAAGCCCACCTTCTCATTTGGTGGCTGGAAATTTGCCTGCCTCTGCCTCTCTCCAGCTGCCTAGCACTGATCTCTGGT
TGAGAGCAGGGGAAACTGAGGTGCCGCTAGGACAAGATCTCATGCAACCGTGAACAGCCCTACTGAGCCATCCTCCTGGGACAGCCTCT
CAGGCCAGGCCTTCTGTGTCTTCTGCTATAGTAGCAGTACAAAGTTGACACAACCTTGCTCTTCCCAGCAGCTGGGAGTTCCTGAGAAG
TGTAAATGAGGTTTCCAGGAAAGCCAAGAGGTGAGAGACTGAAAAGTTGGAGGGATAAGGGGGTGCCCATCAAGCTGCAGTGGAGACTT
GTTATCTCTTCCGCATCCCTGACATCTCAGCAGGAGGATCAGCAGTTCAAGGGCATCCTCAGCAACATAGTAAGTCTATGGAGCTAGCC
TGGGCTACACAGATGCCATTACAAAAAAAAAAAAAAAAGTAAAAAAAAAATCTTGGGATATGGGGGATGGGTGGGTGAACTGTGTAGCC
CAGGCACTCACTACTGGTTCCTTTGGAAGTCCCTCTTCTGCATCGAGATCATGGAGATTCTGGGGACACTCCAGGACTCCTCCTTCAGG
GAACTAAGTTGGGGAGCACCGCAGCCTATCCTGTGGCCTTATTTCCTGTCTGCTCCAAGCGCCAGGCAAGGCATCCAGCCAGGAGCCAC
TCAGGCCTGACCTCCGCTAACTGTTCTGTTTTGTGCTGCACCCTCATCTCAGCAGGGATCAGGTGCCACTTGGGTCCCCAGGGGATCCA
GGGACACAGTGTCCATGTCCCCATCTTTCCCCACAGGCACCTGGGGCAGGGCCGAAGAGGAGACCTCGTGATGTGTACAGGTCTAGCTC
ATCCCTCCGAGGACTCAGGGGACACTTGGGGAAGGTGTGACACAGAGCAGCCTGGAGCTCTGTGCGGCTCTTTATTAAGTGTGCCTACA
ACAGGGGCAGAGACACAGCAGCGACAATGATAACAGCCCACTCCCAACCGAAGCTCAATGGTTACCAGACTCAGCCAACTGCAGAATTC
CAGTCCATAACGGCACCTCCTTTCCTTTCTCAGGAGCTTACCAGAGAGGTCTCACCCCAAGCCTTCACCCCATCTAGAGCAAATGCTTA
TATCCCCCATGTTTGCTCAGAGCAGAGCATGCACCGACCTTCCTGTCACCAGAGGCAGAGGCATTCTGTATACGTGCATACACGCCTCT
GTGTGTGTGTGTGTGTGTCTGTGTGTGTATTTGGTACAGATCTAAGCATAGAAAATTGAAAAGCATTGAGGTTGGTGATTGAAAGCCTC
ACTGATGGGCAAGTCGGGTTCGAGTGCGCTAACTGTATGACTCTATGGATCCGAATATCATGCTGTTTTGGAAAGGAATGAGGGAGCTA
CTGTTTCTAAGGAAGAGAAGAATGGGCACCAAGACATAGGGAGGGAAGGTGGGACAGTAAGCACTTACAGCCGAAGCCCAAGACCTGGA
ACCCAAGGGGAAAAACAGGTGTCGCCAGGCTTGGTGGCACGCGCCTTTAGTCTCTGAGTTCAAGTCCAGCCAGGATTACATAGCAAAAC
TTTCCCTCAAGAAAAAAGGTAGGGGCTGGAGAGATGGCTCAGCAGTTAAGAGCTCTTCCGAAGGTCCTGAGTTCAAATCCCAGCAACCA
CATGGTGGCTCACAACCATCTGTAATGAGATCTGACGCCCTCTTCTGCTGTGTCTGGAGACAGCTACAGTGTACTTACATATAATAAAT
AAATAAATCCTAAAAAAAAAAAAAAAAGAAAAAGAAAAAGAAAAAAGGTAGAGGAGGAGGAAGAGGAGGAGGAGACGGCAAGACAAAAC
TAACTACATAGCTGGGGGAGGTGGTGCTTGCCTATAATCCCAGCACTTGGAAGGCTGAAAGAGGAAATCAGGAGTTCCTATGCAGCTTT
GGCTTATGTGAGCCTTTCTCATAAAACCAAGAGCAAAAAAGCAAAACATGAGGTGGTAACTCAGCTGGAGAGTGCTTGTCTAGATTCCC
TCAGTGAGGGGCTGGGGTGTGACTCAGTGCCTAGAATCCCCCAGGGAGGGGCTGGGGGCGTGGCTCAGTGGTAGAGCCCCTGGCTAGAA
TCCCCCATTGACGGGCTGGGGTGTGGCTCAGTAACAGACTGCTCACCTAAGATGTTCAAGGCCATGACTTTCATTGAAAACATCACAAA
ACAAACAAAACCATCAGTGAGGTTGAATGTGTATCCATGTTTGCTTGTCTCCATTTAAAGAACACCTGGAAGATCTCTAGCTGCTGGTG
GAGGCGGCAGGGTGAAGGCTGCTTCATCCTTTGTATTTGAACTGTGTGGCTGTGGGTGTATTATCTGTTCCCAAGGACTGCACATGAAA
TGAGAACAATGAAGGCTTCGGTTCTGCTGAGATGAACCTCAATTCTCCACAGATGATCTCACCCAGAGCCAGATGGAAGTCCCCCAACA
TTTAAGCCTCTGTTCTGAGTGCTGGACCCAAACTCATCTTAAGGGAAAACCCAAAGAGCCTTTCTGTGGTTTCCTTCCACAACTGTCTT
TCAATGTGTGTATGTGTGCACGTGTGTTTGCTTGTGTCTGTGTGTGTGTACTATGTGCATGTGTGTGAGCATGTGTGTATATGTATGTG
CATGAGTGTGTGTGTGTGTGTGTATACATGTGAGCATGTATGTATAGGCCAGAGGTTAGTCATGGGTAACTTTCTCCACAGCTCTCCAC
CTTACTTTTATTGTCTGTGTGTGAGCCGCTGTGGGCACTGTACACCACAGCACATGTGTGCAAATCAGAGAACAACTTGTGGCTGTCAC
TTCTCTCCCTCTGTCTGGGTCTAAGACCTGGATCCACCGCTTTCGCTTGCTTGAGACATCTCGTTGACTAGCTCCTCCGTTGTTAACAC
AGGGTCTCACTATGTAGGTCCAGTTATCTTGGAATTTGCTCTGTAGACCAGGCCGGCATCAAACTCAGAGATAGACCTGCCTTTGCCTC
CAGAGTACTGGGATTAAAGGTGTGCACCACTACCACCCAGTTTAAAATTCTAAAAAGATTTGTGGGTGCAGGGATTTGAGGAGGCCAGG
AGAGACTGTTGGATCTCTGGGAACTGGAGTTAGAGGTGGTTGTGAGCCCCCACGGGTGCTGAGAACTGAACTAGGGTGCTTGAGAGGAG
CTCACTCACTGCTGAGCCCCCACTTTAGATTTGACTCCCTCGTACTGAAGCTGAGTTCAGTGATTTCGCTGAGCTGACCGCTCAGGGTG
GGCTTTTCGATGTGCACCACAGGCCTGGCTTTATAGGTGAGGTCATGCTTGCACAACTGGCCTTTTGGTGACTCAGCCAACTCCCCAGA
CCTTTTTTCTTTTGCATTTACGTGTGTGTGTGTGTGTGTGTGTGTGTGTGTGTCTGTGTGTGTACATGAAGTGAGAGGGGGCAGTCTGG
GAAGGGTCAGTGGACAACTTAAGGGAGTTGGTTTTGCCTTCCATCATGATGGTCTAGAGACTAAATTCAGCCCCTCAGCCTGGCAGCGT
CCCTTTACCCTCCGAGCTCCCAGCTTTGGGTTTTTGAGGCAAGGTTTCATATAGCTTAGGCTGGCCTGGCATTCAAGGTCATCTCAGAG
GCCTAGGATGACCTTGTGTTGTCGGTTCTCCGCCCTTTACCTCTGGTGTGCTGGCGTTTGCCTTACCACACCCAGGCATCCCCCATAGC
TTAAGCCTAAAGCTCTCGGATCGCCAGCACGCCCATGCCCTGACGTGAAGTTCAGTGACTAATCAATACAGAGCGTTCACGGAGCCCTG
ACACATAGCAGCATTGACTTAACCTTAACAGCCTGGTGTGTAATGAGGCCAACTTGTCCCATTCTGCCCACGATGCGATGCTCTGGGTT
AAACAGGTTTGAACACAGTACCGGATGGCAGGGACGGCACCCTCAGTACAGGGTTTTGGCTGTTACTGCTTCCAGTCTCCTGACCTGCT
CCCCCAAGGCTGGTCTTAAACTCAAAGCAATCCTCCTGTCCCTGCTTCTCAAGTTCTGGAATTACAAGTGTGACTCACTGTACCCAGGA
ATATGTGTCTTTATTACAGAGGAAGCACACCTCAGAGACTGAGAGTAACCTGTGCAAAGTCCCACAGCAGGAGAGGGTGGCTGGCTATC
GGCCGTGAACTTCCTGACCTTTCCTGTGACCTCTAAGGGAGGAACTAGATGTCCCAGCAAGGATTCTAGGGACTTGTCAGGACTCACCT
TTGATGATGGGAAAGCCATCCCATCTCCCCTTCCCCTTTGATGGTCGCTCCAGGGGTTTGCCTGGGACAGAGATGTGTTTCCACACAGA
TCACCCTGAAGACTGGTCAGTTTCAGTACCCTGCCCAGCCTGTGGCTGGGAAGGGCCAGGGGCCCAGATCTTGTGGGCAGCCTGGGGTA
GGGCCAGCATTCCAGGGGCCTGCCTGGCATGTGGGGAATGTCCCAGGAGAAGTCTCAGTCCTCCAGAAACTCAAAGGGAAGTGTTCTTA
CGTGGGGTGGTGTGTGGTGGGAGGTGGGAGGTGGTGTTTGTGGGAATGTCCCTCTGGTCTGGAACCCTGAGGGGCTGCAGTTACCAGGT
ACATTGGGCCTGTGCCATTCTAGGCACCTGACCTGCTTTTCTGATGTCTTCATAAACAGAGACCACTATTCCTGCCTTACCTACCCTGC
TTCCTGGTAGACCAGCACCCCGGGGCTTCAGCATCACCACAGTAGGGACTTTTGTGGGCCACTGACTTGGCCCACAGTTACCACAATTT
ATTTAGAGTCAGCAACGTGGCTGTACTCTATGCTGACCCGGGAATACAAATCTCGGTTTCTGCTCAGGAGGCCCAGGCTACAAGGCCTT
GAAAAACCCTGAGGCGGGCGGGGCAGGTACGGCTCTGCATGCGCCTCTCTAGAAATTAACCTCCTTTCTACCCCAGACCCTTCTCGCGT
CTCTGGCTTCTTCAAGGACCGGCTCTAAGGCTACTTCCTGCTACACCCTGGACCCTCCCTCCATCCTGAAGGGATCATTATCTTAAACC
TGGTCCTTCCCATCGACTGGGTTTGGGGGTAGGCGTGTTTCAGGATGCCCTCAGAGACCAGGAGAAAGTTGGAGATCACCCAACTACTG
CAGAGCACGACTGTGGAGGAACAGTTAAATGGACTAGCAAACACGAACGGGCCAGAGGCAGAAGGACTGCTATGAGTTCCAGTCCAGCC
TGAGCCACACAGATACTTCAAGCCTAGTCTGAGTTACAGAGTGAGACCCGGTCTTAAACCCCAGAAAAACACTCCGGGGAAGCGCAGGG
GACCGGGCGCGCGCTGCAGAGCCCCCTCCCCGGCAGGCCCGGGTAGGGGCGTGGCCACGGTGACGTCATCCTCCTATAAAACCCTGGGC
GCCGCCGGGCTGGCTTTGTGGAGAACTGCAGCCGGCTAAGCCGTGTTGAACAAAGGACGTCGGGCACACCTATCCAAGCTCCCGCGGCC
ACCCGGCCGCCCTCCCCCACCATGACCGCCAACCGCACCGCAGAGGCTGTGCAGATTCAGTTCGGGCTCATCACCTGCGGCAACAAGTA
CCTGACAGCCGAGGCGTTCGGGTTCAAGGTGAACGCATCCGCTAGTAGCTTGAAAAAGAAGCAGATCTGGACGCTGGAGCAACCTCCCG
ATGAGGCGGGCAGCGCGGCCGTGTGTCTGCGCAGCCACCTGGGTCGCTACCTGGCCGCCGACAAGGACGGCAACGTGACCTGCGAGCGC
GAGGTGCCCGACGGCGACTGCCGCTTTCTCGTCGTGGCGCACGACGACGGCCGCTGGTCGCTGCAGTCCGAGGCTCACCGGCGCTACTT
TGGCGGCACCGAGGACCGCCTGTCCTCCTTCGCGCAGACCGTGTCGCCGCCCGAGAAGTGGAGCGTGCACATCGCCATCCACCCGCACC
TTAACATCTACAGCGTTACCCGCAAGCGCTACCCCCATCTGACCGCGCGGCCGGCCCACCAGATCGCGGTAGACCGCGACGTGCCTTCC
GGCGTCGACTCGCTCATCACCTTCGCCTTCCAGGACCAACGCTACAGTGTGCAGACGTCCGACCACCCCTTCCTGCGCCACGACCGGCG
CCTTGTCGCACCCCCGGACCCCGCCACGGGCTTCACGCTGGAGTTCCGCTCCGGCAAGGTGGCCTTTCGCGACTGCGAAGGTCGCTACC
TGGCTCCGTCCGGGCCCAGCGGCACCCTCAAGGCTGGCAAGGCCACCAAGGTGGGCAAAGATGAGCTCTTCGCCCTGGAACAGAGCTGC
GCTCAGGTGGTGCTGCAGGCGGCCAACGAGAGGAACGTCTCCACGCGCGAGGGTGAGTTGGGGACATGTCCCCTCCCTTGCGTCCTGAC
CCAGTGCACAAAAGCATCTCTGCACTCCTTCTATTTCCCTATTTCCTCCTCGGCTCCCTTTAGGCTCACAGGCCTCTGGGCTCCGCCAT
GGGGAGGTGGGCTTCTATTTTGAAAGACCTGATCTCACCAGATAGCCTTGGCTATCCTCGAACTCGCTATGTACACTGGGCTGGCCCCG
AACTCGCAGAAATCTTCCTTCCTGTCTCTTCTGCTGGGAATAAACCCATGTACCACCCCTCTGTTCAGGACGTAGGCTTCTTCTCCTGG
GAGAAGGTGACGTGGGGAAGTGGGTAAGGCCTAACACATTCTAAAGCAATACTGTCCACCACCACCCCCCCACCCCCCCACCCCCCCGG
CTGGGGGCGTGGGACTCCATAGACCTAGCTTAGATCTTCTCCGTTTCCTCTTCCTTCCCAGTCTGAGCCATCCCCATTCATGAGCCGAT
TCTTTAGGATAGGCTGTCCCAGGTCTTGGAGAGGAAGGGTTCCCCCTACACCCCCCCTTGTTCATTTCCTCCTGCGTCCTCTCCCGTTT
CGCGGGCCTTTGGCTCGAGGGCCAGGGGACAGTCACCCCTGCCCTCCCCGCCTTAAATGGCCAGATAGAGGAGGTTCGAGCGGCTTTTG
ATCCCGGCGGGGTGTGCTGGGCCGGCTCGGCCCTGCGACTCTTGGTGCTGGTGGGGTTGTCCCTAGGGGCTTCCCCTCCCCCACTCCAT
CTCCCCTCGCTAAGGCCTGGCGCTCCGGGCCGCGGCCTTTGTGAGCAGGGGCGCGGGTCGCCACGGCTGGGCCCTCCCTCGTCCCCTTT
GTCCTGCTAAGGCTCTGCACATCGCAAAACCAGATGCGGCGGACCTGGGGCAGGGGATCGGCTTACGCGGGTCCCTCTGCGGAAGACCC
ACCCCCAACCCTCCAGGCGTTGTCCCTGGTCCAGGGTTCATTGAGAAGGCGCGGAGTGAAGCCGCCTGGCTCTTCCTTCGCACCCCGGC
TGGCAGGCGGCGTGGCGCCGGTGGCTGCGCGCCAGGCGCCCTCCCTGCCCTCCTATGCACGCCTTGCGGTCCGGCACTCGGCGGACTGT
CCCTCCAAAAGCCTCTACTCTCGAGTGAACCGGTCCCCAAGGCCTCTCTGGCTGGGAACAACCGTTTGCACCTCTATTTGTCCCCATCC
AGGGCCTCCAGCACCCTGGCAGGCCATTTCCGACCGGCATGGGCATGGTACAAGTAGGCGCCTCCTTTCTGGCGTACCCCCCACTCTTC
AAAGGGTCTTTGCTCCCTGCTGGTAGGGGCTTGTGAGATCTCGCTTCCTTGGTTGGGAAACTGAACCCCAGGCTTTGGAGATAGCTGGA
TGGGAGCCAGTCTGTGGGGAGGAAGACCCCCCTCCCCCAATGCAGAGGGCAAAGCCTTGGGTGGGGCCTGCTTGAGTTTGGCTGAATAA
CGCTCAGAAAAGTGCGACCATTTGCTTACTGTTACACAGTAAGTCAGCACCATGCTGAACTGGGTGTACTTGATGCCAGAATGTTCTCT
CATTATACACACACACACACACACACACACACACACACACACACACCAATGAACCATGGCCTCTCTTGTTTCACAGTGCTGGGCGAGGG
GGTCAGAGTTCCCACCTGTTTAGCAAACATTGAGATACGAGCTGCGGTAAACGTCTCTTGGGAATGTGACTGGTATGTAAAGCAGAAGA
TTGGGGGTACAGGATCGGGGTAGTAGATTATAAAGATCTGGGGTCCCTTCTGAGGACCCAGGAGCTGCCCCAGGAGAGTGAATAGAAAT
GAGCATGCACCAAGTTTATGGACTGGTTTTGCAGGCAGGGGCCTCAGCAAAGCCCCGGAGGCAGGGAGTTGCAAATAGAGAAGCCCTGC
ATAGCTAGGAGGAAGGCTCGGATTGCACGGAATCCTGCAGATCACGGGTACTCCAGAGCCTAGAGAGACCAGCTCTCTGCTTACACAGC
TGTGGCCGAGGCTTGTGCCCTGGGGAAGGTTAGGCAAGGCACAGGTGACTTGGGCCCTTGGGGCCTGAAACGAGCTAGAGGGTGTGTTT
TGCGAGCCTGCACAGGTACCCTTTTCTGAGGGTGCCATGATTGTGCATGATTCACACTGATGCGGCAGGAGGGGGCGGCAATTGAAGGG
GTGAGAATCTGACGGGAACTTGGGGTCACACACAGTCACACCTCTTCTGCCCTCCTATGTCTGCGTCCAAGTCTCATTCCCGAGTCAGC
CAACTCCTCCCCCGGCTTTTTGAGGGTCAGCTGTTGGGTGGGTGTGCCGGCGAGGCCATTTTGGGCAGGCTTGCGTGGCAGGGAGGCGG
GGCTGGGCTTCGGGGAAACACGTGTTGAACTGCTACCCAGGAGTAGGGACAGGGAAGGCTCAATCTGGTCTGGGGTTGTGAATCTTCCA
TGCAGATTTGCTGCTTCCGGCTTCCTGGCCTCAGTTTCCCCATCTGAAAGCACAGAGGTACATTGAGCCAGGATCTTCCTGGGAGGTGG
AGATCCTATGAGAGTAGGATGGGGGGTAGTGGGTAGGGTCTTTTCCTCCTTGCTTCTCATGACCCTCCCAAGCCCCTGCTGGCTCAAGG
ACACCCCGTGCTATGTCTCCCCACACTGCCCACCCTGCCTTACTTGTTGGGTTCTAATGCTTTCAAAGAATTTTGTCCCCATCATTTCT
AGGGCGGCTGGCTAAGAATGAGTCACAGGTGAGGCTGGGCTTTGTACCGTATCCTTTTGACGGCTTCCCTCTTGGGTCGAGGAATGGCT
TAGTTTTGAGCACTGTACCACTTTGGGCGGTGAAGTCCTGGAGGGTTCTAAACTAACCCGCCTTCTGGGTTGTGAGGATTGCTTATTTC
GAACAACAACGCTAGGCACTCTGGCAAACAGCTAAGCCCAGGAAGGGGGTTGCGGGTTAGAGGACCCTCTGAGTCACCGCAGAGACGCC
TAGGAAGCTGGAAGACAGCCACGCCCCCTCCGTGCGCTCCTAGGAGCCAGTGACTCAGGTTTTCTGTGCTGCTCCTGCTGAAAAAAAAA
AAAATCCCACAGAAAACCTGTTACCATAGCAACGCAGCTCCAATATGATTCCAAGGCCAAGTGGGGCCTTTCCCCCTGGCTGAGCCTTA
GAGGGCCCATTCTGGAAGGGGGCTAACTTCCCTTTCCGGCTGAGGTGGCAGGGCCCACCATCTCCTTGGCAACAGGCCCTCACGTCCTT
TCACGGTCCCAACAGAATTGAGAGGCGATTCTTTTTGGCAAGGGAGGGAAACTGAGCCAGAGAGCGGAGAAGACATTCCCCGAACGGTG
CCTGGAGTCCACCTTTGGTCCCTTGGTGGCCTCTTTATTAAAAAGTGGTTGCACAGGCCCCTTTGTTCCAGTACCTGGACATGAGACCC
ACATGGTCAGGCCCTTTTTGTTCTCTGTCTCGACGTCTCATTTTTCAGAGCTACTGGGAGGTTGAAGGTTGGTCTATGTCCACAGCACT
GTGCTGTCTTGACTCAGTGCGCATGACATCTTGGGCCAGGGCCTCTCCTTTGTGTGAGGCAAAGCTAGCATGCTGGGAAACTCCCACTC
CTTTAGTCCACCCACTTTAGTTGTACTTTTTGTCTTGTAGCCCATCTGCCTTCCAACTCTCCATCCTCCTGCCTCAGTCTCCAGGGTGC
GGCGATGACACGCATAGGTCACCACAGGCTTCTCGCCTCCACTTGATGGTTGCCGGAGTGGCTGGGAGTGGCTTTTCTTTTCTTTTCTT
TTCTTTTCTTTACTTTCAGGCAGGGTTTATCTGTGTAGTCCGGCTGTTCTGGAACTATGTNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNAGAGAGAGAGATTG
GGACTGACTGGTCCCACTTCTGGCTTTCCCTGACTGTTCCATTCCCCGCAGTGGGCACGGTTAGATCAGGTGGGGTCACTTACGCTCGC
TAGCATTGGGTGTGGCTCTTTTCTGCCCAGTATGCTGTGCTAACTCAGCTGTCCCTGCACGTGGCAGGTCAATTACAGGTAGGCTCAGG
GGACAGTGCGTGACACCCTGTATATGTGTAGCCAATTGTTCTCTTAATATTTACCCATCCCTAGTGTGGGCCACCGGCACGAGAGCCAT
TTCTGCCTGAGAAGGTTGAACAGACTACAGCCCAGAGCTGTGGCCTTGGGGTGGCTGTCTAGCCTGGGATGGCCCTCCTACTCTCCTCT
GTGCTTCAGTGGTAGATCCTGTGGAGGCACTGGGCCAGGGTCCTTCCGGAGCCATCATGGAAGCCATGTCTGCTGATCCATATGTCTAG
AAGGGGAGGCATCTGTCCATCCCCACTCCTGAAGTCAAGGGACCCCTTGGTCTTGCAGGGTGACCTTGGGCCAAGGCCAGCCACCTCTG
AGCTCCATCTTCTGCTTTGTGAGGTAGAGGTAAGAGCCTTTGGCATTGACAACACAGATAGAGACTCGGTTGGGTCAGGGTGGCATTGG
CTCGCATACTCTTGGGTGTAGTGTCAACTAAGGATGAGTTCCTAGGGGCAAGGCTGAAGGTGGCATCTACCATCCCATACCACGTTCTG
CTGCCTAAGGACGCCTGAAGACAACTGTGACACTGGCAGTGTACACTGCTTCCAAGCTGAGGACATTGTTAGAATCCCAGATCCTTGAA
ACAATGAGTTTATCTTGTTAGGACCCAACATCCCACTCCTACCTGGAGTCAAAGCCTTGGGAGCGCCACCTTATTGCGCCTCAGTCAGG
TCATGTGACATGGAAACCATGTAGTGTTACTATGTAGCAAGAAGTTAGAAAGCCATGCAACAAACCTGTAATTCCAACTCCTGAGGCAG
AGGCAGGTGGATCTCTGATTTCAAAGCTAGCTCCAGATACACTGTGACACCTTCTCAAAACAAGACAAGCAGACTGGAGAGATACCTCA
GAGGTTAAGAGCACTGGCTACTCTTCTAGAGGTCCTGAGTTCAATTCCCAGCAACCACTTGGTGCTCACAACCATCTGTAATGGGATTC
GATACCCTCTTCTGGGGTGTCTGAGGAGAATGACAGTGTAGTCACATACGTAAAATAAATAAATCTTTAAAAAAAGAAAAGAAAAATGA
AAACGCAGGGGGGTGATCGAGACATGGCTCACACAGTTTTTTGTTTGTTTTGGCTTTTTGGTGTCTTTTCTTCTTTCTTTCTTTCTTTC
TTTCTTTCTCTCTCTCTCTCTCTCTCTCTCTCTCTCTCTCTCTCTCTCTCTCTCTTTCTCTATCTTTTCTCTTCTTGTCTTGTCTTGTA
TCTGTGTAGCAGCCCTGGCTACCCTGGAACTCACCTATAGGTCAGGCTAGCCTTGAACTCACAGATCACCCTGCCTTTGCCTTCCAATG
CTGGGTTAAAGGTGTGCTCCAGCACTGTCCACTCTGGACCACTTGTTCTTGTATTCACCTAGTTCTCACAGGGTGGCTCATGACACTTC
CAGCCGACCTCACCTCTTAGGGCACCAGGCACATGTGCGCATATTCATACACACAGGCAAGACGTTCACACACGATACATACAATACAA
GACGTATCTTTCTAAAAATTAAAGAAAATAAATGAGAAGCGAGAGGGAAAGGAGGGAGAGCCACTGTTTGGACTGGACCACCACTGGCA
GGGAAATGCCAGAAGGTTCAGACAGCATTCAGGGTTTGTGGCTGGTGGGGGCAGGGATAAAGGAGGACTTTTCTAAAGGAACATGTGGT
CTGTGCGTCTGTGTGTGTGTGTGTCTGTGTGTGTGTGTGTGTGTGTCTTACTACGGGTACACTCCAGGGCCTTCCACCATGCTGGGCAA
ACAATTTACACTGAGCTATATTTTCGACCCCCTCTTTATTTTATTTTTATTTTATTTTATATATAGCTCTGACTGGCCTAGAACTCATT
ATGTAAACTAAGGTGACCTCAAACTCACCCATACTCAAATGAGATTCACCTGCCTCTGCCTCCTGAGTGCTAGAGACAGTCATGAAGCA
CCACGCCCACCTTCTCTTTAAAAAAAAAAAAAAGCCTATTGCTTTTGTTTTTATTTTTGTCTGTGGAGGGTGGTGCCCACACCCAGTGG
TGCCAGTTTGGAATCAGTTCTCCTGCCCTATTCTACCTAGCCATCGAACGCAGGTGGGTAGGTTTGGCAGCACCTGCTTTTACCCCACG
ACCCATCTCGCTGGCCTCTAGCCCTTTGTGTGTCTGTCTGTCTGTCTGTCTCTCTGCATGGAGTGGAGTCTCATCTAGCTCAGGCTGGC
CTGCGAGTTATGATCTTCCTGACCCTGGTATCCAAGTGCGGAGACTGCAGGCTCACTCTGCCACGCCCTTTTACACAGTTCTGGCATGG
ACTCTGCATGTCAACTGAGTTATAGCCACGGCCCTATTTTATTTTATTATTTTTGAGATCGGGCCCCACTCATCATGGAGGCTAAGGTC
GGCCTCCTCAGTGGTGGCATGACAGGCGTGGGTCGATGTGCCTGGCTCCTACTCTATTCTTTTACTTTCACACATACCCACTACAGGAA
GCGAGTAGCGAGCCCCCTTTTACCAGCACCCCGCCATGTGACATTCCGCAGAACCAGCGCCATCTGTCTGTTTATTCAATGGTTTTGGT
TGTATTTAAAGCAAATTCCCCACACTGGTGCTTCTTACCCGGAAACGCCTCCATCTCTAGCTACAAAACCAGGCCATTCCTTACATAAC
GGCAGCGTTGCCTAACTGGAACCGATCTTTAAGACCCAATCCAGGCCCACGGGAGAGTTCTGCTACGCCTTAGAACATCTTTGTGGCTC
GTTCGTTTGAAGTTTGATGGCTTCCTTCCTCCCCTGCTCCCCTTTCTCCTTGCCATTTTGGGCACTAGAGTTTAAAGAAACCCGCCTGC
CACTGAACTAAATCCCCAGATTTTTTTTTTTTTAAGTTTTTTTTCGAGACAGGGTTTCTCTGTGTAGCCCTGGCTGTCCTGGAACTCAC
TCTGTAGACCAGGCTGGCCTCGAACTCAGAAATCCACCTGCCTCTGCCTCCCGAGTGCTGGGATTAATGTCTTATGTATCCCAGACTGG
TCTTGAACTTGTGCTCATGCCTGGAGTATCCTATTTTGATTGTTTATGTGTATACCCCTAATATGGATGTCAGAGATTCATATCATATG
TGCCGTCTTTGGTCATCCTCCATACTTTTGGGGGATGTATGGGGGCAGGGATGGAGTCTAGCCTCGGCTGTGGTAACACACGCTTTTCA
TTCCAGCATTAAAGCAGAGATAGGTCGATCTCTGAGTTCTAGGACAGCCTGGCCTACACAGGGAAATTCTGTCTCAAAAAAAAAAAAAA
ACAAAAAGAAAGAAAGAAAGGAAAAGAAATAAGAGTCTCCCTATATAGCCCTGGCTGTCCTAGAACTTATTATGTAGACCACGCTGGTC
TTGGACTCACAACAGAGATCCATCTGCCTCTGCCTCCTGAATGTCCACTCTCTGCTTGGAGACAAGAATCGCTCAGTGGCCCGGATTCA
GGGATTCAGCTAAGCTGGCTGGTCCCTGACCTCCAGGCCTCCATCTGTCTTGGCTTCCCTGTGACAGGATTATCGGCTCATGCCACCGC
GCCTGGCTCTTACACCATTCCTAGCGATCTGAATTTAATACTCTATGGTCAAGCCATCCTTCAATGCTCCCTCCCCTCCTGTTGAGACA
GAGTCTTACTCACTCGGTAGCCTCGGGTCCCAGAACTCACAGTTAGTCTCCCGTTTGTTTCCTGAGTGGTGAGATCGTTCCTGGCTAGC
CTTGGCTGCTGATTTGGTAGCCTGGTCCCGCCCACTGCTTAGGATTATCACTGAGGAGTTTAAGCCACACTTCAGGAAGCCCTGTGGAT
CCTGGAATCTGAGTGAGACAAGGGTCACACAGTCGCCTGACCGGCTCTGGTCTCTCTCTGCAGGAATGGACCTGTCAGCCAATCAGGAT
GAAGAGACCGATCAGGAGACCTTCCAGCTGGAGATCGACCGCGACACAAGAAAGTGTGCCTTTCGCACCCACACGGCCAAGTACTGGAC
ACTGACGGCGACCGGAGGTGTGCAATCCACTGCGTCCACCAAGTGAGTGACACCCTACACCCCTTCATCACCTGGCCTGGCTCTTCCCC
CAGGACTGGGCAGCCTGCTCGATGCCCCCACGTTGCCAGCCCCTCTTCTCTTCCCCAGGAACGCCAGCTCCTACTTTGACATCGAGTGG
TGTGACCGCCGGATCACTCTGAGAGCCTCCAACGGCAAGTTTGTGACCGCCAAGAAAAATGGCCAGCTGGCCGCCTCGGTGGAGACAGC
AGGTAGTCACTTGGCGGCACGTACCCTAAGCCTGTTTCCCTAGTACCCCGTGGTCAACCATCAGTCCCACCTGGACCTCTCTGTGTGTT
CAGCGAATCCCTGTAAGCTGCTGTACCCTCAGGCCAGCAGCCTGTGACCCTTAGCTTCTTGGTATCCCTCTCTGCTGAGCCATTCCCTG
ACTGGCCCATCTTTGTTGCTGTGAAGCTCACTCCCTCCCTTTCCCTGTGGCAGGGGACTCGGAACTCTTCCTCATGAAGCTGATTAACC
GCCCCATCATCGTGTTCCGGGGGGAACACGGGTTCATTGGCTGCCGCAAGGTCACGGGCACTCTCCATGCCAACCGTTCCAGTTACGAT
GTCTTCCAGTTCGAATTCAATGACGGCGCCTACAACATCAAAGGTGGGTTCACTGGGTGAGGATGCACCTGGCCATTCAAAGCCGACAT
TAGGGAACGGGTGTCCTATGACCGCCTGGGGTAGCCCCTCCCCCCTTGTCTTAGAACTTTCCTGGCCCAACCTGGGCAGACAGCGAAGG
TGGGAAGCAGCTAGGGAGAGTGGTCGTGGCTCCAATCTGGGAATCGCACACAGGAGAATTATCTTTTTTTTTTTTTTGGTGTTGAGGAC
AGAACCCAGGGCTTTGAGCTTATTAGGCAAGCATTCTACTGCTGAGTTAAATCCCCAACCCCAAGAATCATCTCAAAAAGAAAGAAAGA
AAAGGGAAAAAGAAGGTCGAGCCTGGTGGTGGTGGTGGCGGCGGCCGCGGCACACGCCTTTAGTCCCAGCACTCAAGAGGCAGAGGCAG
GTGGGTCTCTGAGTTCTTGCCAGCCTGCTCTACAGAATACATTCTAAGACAGCCAGAGCTATGCAGAGAAACCCTGTCTCAAAAACAAA
CAAAAAAAGGGGGTGGGGAGAGAAGATAGAAGATAAGGATATTCCTTCTTAGCTAGCTGTGGGACCAAAACCAGTGAGAGGACACAGCA
ATCCAGAGGTGTCAGTCAGAATCAGAGCCCTGTGCTGTGTGTGGCCTTAGCAAGGCAAACGGGACTCCTTTCTTACAGTGCTGAGGACT
GGACTCAGAGCTAAGATCCAGCCCCTCCCTGCCGGATTCTAGGCAGCGGCTCTACCACTGACCCACGCCCCCAGCCCCTCACTGGCGGA
TTCTAGGCAGGTGTTCTACCACTGAGCCACGCCCCCAGCCCCTCACTCGGGGATTCTAGGCAGGGGCTCTACCACTGAGCCACACCCCC
ACCCCCTCACTGGGGGATTCTAGGCAGCGGCTCTGCTGCTGATATATGTCCTTTTTGTTTGTTTACTTGTTTGTTTGTTTGTCTTATTG
TGGCCCTAGCTGTCCTAGAACTTGCTTTGTAGACCAGAGATATACCTGTCTCTGCCTCCCAAATGTTGGATTTAAAGGCATGTGCCACC
ATGCCCCACCCTAGCCTACCCCTCTTATGTTTTGTAGTTTGAGATGGCATGTCAGTAGATTTCCCAGGATCGCCTTGAATCTAGGTAAT
CCTCCTGCCTCAGTTTCTTGAGTATTTGAATTATGCGACTGTACCTTACATCCAGCTGAGCCCACTCAGTTCTGGAAGCTCACAGCGGG
AGGGAGTGTTAATCCTTTGGGGACTGACAAGGTGGGGTTAACATGTAGTCTCCCTCCTCAGACTCCACGGGCAAGTACTGGACGGTCGG
TAGTGATTCCTCGGTCACCAGCACCAGCCACACCCCTGTGGATTTCTTCCTTGAGTTCTGTGACTACAATAAGGTGGCTCTCAAGGTGG
GCGCCCGCTACCTGAAGGGCGACCACGCTGGCGTCCTGAAGGCCTGCGCGGAGACTATCGACCCCGCCTCACTCTGCCAGTACTAGGGC
CACCTGCCCTCTGCACGCCGCTCTCGTCAGTCCCTCCTGTTATCCTTACTCATCGGGTGGCCCTGCAGCACGTGGCAAACCCCTTGCCT
TTCAAACTGGAAACCCAAGAGAAAACGGTGCCCTTGCTGTCACCCTCTGTGGACCCCTTTTCCCTAACTCACTGCTCCCCATGGGTCGG
TGGCTGCAGACTGTCCCCAGGAGGGACTCTGGTTCCCTCTGTCCCCTTCTTTCCATGGGGAACTCTGGCACCTTTCTTCTCACCTCAGT
CAACTCTGACCCTTATTTCCCCCCAGGAAGTGGCCTAGGAGAAGCTACAGGGCCTAGGGACTTACCCTGAGCTTGTAACTGGAAGACCC
CGTCCCTATCCCCGCTCCCGCCCCCACCCCACCCCACCCCTGCTCTGCCCCCAGCCTCTGGAGGCCAGCCTTTTGGCGGGACTGAAGCC
CGCCATGGCCAACCTTGCCCACAAGTCTTTTTCTGGATCTTGGCTGGAAGGCACTCTGTCCCATCCTGCAGTGTTTGGGCCTGGCTCTT
TGACTCAAAGCTAGCTACGTGGCACTCCGTGTCGCTCCTGCACATTCTGGAAGGGGCGGGCCTCTCACCCACCTCATTCCTTTTCCCCC
TGGCCTGACTGGAAGCAGAAAAATGACCAAATCAGTATTTTTTTTTTTTTTCTTTAAGGAAATGTTACTGTTGAAAGGCCCTAGGCAAG
CCTGCCCTGTTGGTTGTAGTCGTGAGTGGTCTTGGGGGCACATGCTTGGCTCCTGTCCCTCCCTCCCCAGCGGGTTCCCTCCCTCCCTC
CTGCCTGACCACCCCAGCTCTGGCTCTGTGATTGGTGCTCCACGTCTTCCCAGACACCTCGGGGCTCCTGGGCGCGAGAAAGCCGGATG
TGCCCCTCCCTCGCAGCCCTCGACTAAACCTCACGGCGCCCTTTCCCAATCACCCCCTTCCACCGACCCCTCAACACCATCCATCTCAC
TCTGGGTGTCTCGCTCCTTTATTTTTTTGTAACTCTCATTTCTATAACTCTGAAGACCCATGATAGTAAGCTTTGAACTGGAAAATAAA
GTAAAATCAAGTCTGCGGCCCGTGTGTGTCTCAGGGGAAGTGGATGCTGGAGTGGGCAGAGGGCCGGCTGGGAGGGAGGCAGCGCTGAT
TGATTCCCAGCCCGCAGACTCTTGTTTCGGTCTCGGACCCTAGCCGCTTCAGCATATCCCTTAGTAACTCTGGACTGAGAAATGGCTGA
CACGAGGTTCCCCAGCCTTACAAGCTGGGCAGGCTACAGCGTTCAGCAGGTAAAGTTGTGTGCTCTCAAGCTTGATCAGACTCAGTCCC
CACCACCGTGTGAAAAGTTGGGCACAACGCTGTACACCCACCCCACCCCCTCTGGCAAGCACAGAGACAAGAGGATCCTTGGAGCCTCC
TGGCGGCCCAACCTTGCCCAATCTGTGATTCCCATTGACTGAGCCATAAGGTGGAGATGGGTTTTGGAAGATACCCCACATAGGCCTGC
GGCCCCCCACACCCGGAAATAAAAACAACCCAGCCTTGTCATCCCAGTTATTCGGGGAGCTGAGGCAGGACGATCACAGATTTAAGACT
CCCCACGTTGAAGAGTAAGTTCAAGGGCAGCCTCCACATCTTGCTACATTCTTTTTTTGTTGGTTGGTTCCTTTTTGTTTTGTTCTTTT
TTTCGAGACAGGCTTTCTCTGTGTAGCCCTGGCTGTCCTGGAACTCACTCTGTTGACCAGGCTGCCCTCGAACTCAGAAATCCGCCTGC
CTTTGCCTCCCGAGTGCTACCATTAAAGGTGTGCGCCACCATGCCACGCTTAGATGTTTTAAATGAACGGCTCTGGAGTGTGGTTCACG
GGTAGCCTCCCTCAGACAGGCTGGCGCAGGACAAATGGATCGCTCAGCATTGGCTCCTGCCTTAGGGTTTCCATTCCTGTGAACAGACA
CCATGACCTGGCAGCTCTTCTGAAGGACATTGGACTGGGGCTGGCTTACAGGTTCATCAAGGCAGGAAGCAAAGCACAGGCATGGTGCA
GGAGGGGCTCAAGTCTACGACCCGGGTCTCAAAGCCCACCCCCACAGTGACAGACTTTCTCCAACAAGGCCACACCCACTCCATCAAGG
CCACACCTCCTAGTAGTGCCACTCCTTGAGCCAAGCTTATTCAAACCACCAATCACAACTGCTTTTCTAGAGCACCCAGCTTCAATTCC
CACCATCCACATGACAGCTTACAATTGTAACTACAATTCCAGGGGATCCAACACCCTTACACAGGCACACTTACAGGAAAAGCACCAAT
CAGTGCACATAAAAATACATAAATCGTTAGAGAGAAGAGACCATTAGCTACCTTCACCCTTAACAGGCAAGCTGAGGTCCTGGGAGTGT
CTTAGAGGTCCGCAGCTTGCAGTCTATAGGGTAGTGGCATCGTCTATACCTTCCTTCCTTGACAGGTAGGTACTTAGCTGAGACCTCTG
GGGTATCCCAGAGACCCATTTTACAGAGCGAGGCACTGAGGCGGAGGGAAAAGACAACCTTGTTTGCTGGCTCACAACAGTGCCAGTGG
GGTACCCACCCACAAGGCTCAGCATCCCGAGGCACTGTGGAGGCAGTTACAAAGCCGCATTGATTGGCTGCTGATGACGAAATGAAGAG
CCCCGCGGTGTGATTGGCGGTCCCACACGGAAGCCATGTGGGGAGATTCACACGGGCAGATGGCCACAAAGGGTCCAGTGACTTCCCGC
AGGGTGGAAGCTCTGGGCCCAAGCCAGTAAAGTGTCAATGACATGCAGAGACAGATGAATCCTTCCCAGTTGAATTCTCAGGCTCTGGT
CGGGCCGCGCTGGGGAGAAAAGACACCCTGTCCGTTACAACCACCAAGCGCAGCCAGGCTACGTGATGACTGGGCCAAGTCCCAGACGT
GACTTCCTCCAGCACTGGAGGGGAGGGTCCGACGGAGGAGGCTGAGAGTCTGTTGGAGGCAGCCGAGGATCGGACGCAACAGGCACAGG
TCTTACATGGGAGGCATAAAGTCTGGTGGGAGTCCAAGGGAGGACGGATGTAGGGTGATGGGAAGAAAGGACCTAATCCAGAAAAAGGA
ACAGAAGAGCCACCTACCTGGTTAGGGGACGCTGGCCCTCCGCAGGGAAAGCTGTCAAAGAAAGGCCTGGCTAATGAGACCATGTCGTG
GGGGGCCCAGGCAACTGTGGAGGCATCAGACACCTTGGGGCAAGGAGGGCACCCATGACCTAGTACACACCACTAAGTATCTCACAGTC
CTGCACACACACTCATTGACACACGTGGGTGAGCAGCACCCACGGCCAGCTGAAGCTGAGTATCCTGCCCGCCCCTTTGTTTCTTCCAA
CTGAGCAGGGACAAGAAGGATCCCTATGAATATGCCTTTTTTTGTTTGTTTGTTTGGTTGGTTGGGTTTTTGACTTGCTTTGGTTTTTC
CAGACAGGGAGTAAAGACATACACCACCATAACCCAGCTTTGTAAATCTGGTTTTTGTGTGTGAACCTATGAATTCCACTCCTGTTGCA
CTGATGGGAAATTGAGGCAAAGTCTCAAAGAAGCTGACAGCACAGGCTAGTGTGTTCATTTTCTCTCTACAAAAAGAGGAAAAATTGGG
GCTGGTGAGATGCCTCAGCGGTTAAGAGCATTGACTCCTCTTCCAAAGCAGGTCCCGAGTTCAAATCCCAGCAACCACATGGTGGCTCA
CAACCATCTATAATGAGATCTGACACCCTCTTCTGGGGTATCTGAAGACAGCTACAGTGTACTTAGATATAATAAATAAATAAATCTTT
AAAAAAAGGAAAAATTATTTATTATTTTATTATTAAATTAATTGTTGTATTTATTTATTTTATTAATCTATATTTAATTTATTTTTTTA
TTTTGTGTGCATGAGTGTTTTGCCTGCATGTGTGTGCACTACCTGTGTGTCCACTGCCTATGGAGGTCAGAAGAAGGCGTTGGGTCCGG
GAACTGGGGTTGTATGGTTGTTAGCCGCCGCGTTCATGCTGGTTCTCTGCGAGGCGGCACTTAGTCCTGAGCCATCTTCTGTCAGCTCC
TCTACTTTCTTTTAGACAGGGTCTCCTGTAGCTCAGGCTCGGCTTCAGCTTGCCTCGTGGTTAAGGGTGACTAAGTTAAATCATCTTAC
ATTTCTGCCTCACTTCCACCTCCTGAGTGCTGGGCTTGCAGGCATGTGCCACTGGGATGGGTTTTGTTTCCTGCTGCCTTCTCACCCCG
TAGCTCCGTGTGTTGGGTCAGCGCCACCCCTGTGCCCACTGAGCCATTAACCCTACTGAGCCCACTGTAGGGAAAAACCATAGAGGAGC
TTCTATGACCCTCATGGGTCCCTCCAGGTCTGTCACAGTTCAGGAGGCATGGATCTCTCTGGCAGGGAGCTGTTGACTTTAGCCTTGGA
ATTTCCCAACCAAGTCTACACTGAGCACCCTGTCGACGACCTGCTTCTGACTCCTCCTGTGAGCCTCTTTTGGAGTGATCTTAGTCCCC
ACAGGGCCCCCATCTTCTCCAGAGCTGAGTGGCCTCTCCTACTGGACAGAGAAGAGCTGTCCTGACTTGGTTAAAATGTTGAATGCAAG
GAGCGGAGGAAACGGCCCAGCTGGTGAAGTGTTTGGCTCGATCCCCAGAGCTGTGGAGTTCCCAACAGGATGGGGCTCACTGGCTGCCC
TGATCAGCAAGCTCAGAAACCAAGATGGATAGCTCCTAAGGACTGGAATTGGAGGATGACCCCTGGTGACAGACACACTTGCATATGCA
TCTGTCTGCACACATACACATATGCATGTAAAACCCTGATCCACGTGGTAGTGCCTGACTGTAATCCCAGTACTTCGCACACAGGAACA
CAGAATCACCTTCAGTTTCAGGCCAACCTCGTCTACATATCCAGCACCTTAGGAGACTCTGTTTCCAACTCTTCAGGGTGGCTCACATC
TCTAATCTCAACCCTCAAGAAGCCAAGACTGGAAGATCCCTAGAGAGGAAGGTTTGCTTGGGTTCAGTGCCAGCCTCCAGAGGCAGAGG
CAAGCAGATTTCTGACTTCGAGGCCAGCCTCTACAGAGTGAGTTCCAGGACAGCCAGGCACCCAGGGCTATACAGAGCAACCCTGTCTC
AAACAACAACAATAACAACAACAACACAACAAGAAAACTTGAGTGTGCGTGTGCGTGTGCGTGTGCGTCTGTGTGTGTGTGTGTGTGTG
TGTGTGTTTACTATTTACAGTAAACGAGTCTTTGGCGAGGCAGCCAAAGTAGGCCACGAACTCCTTGTCCCCTGCTGGGCTCTCTGGGT
TGACATCTTGTCTTGATGGCGAGATAGGAAGGGATTCTCTGTCCCTCACGGTGCTATCAGCAGTGCGAACAGATGGGGTCGGGAGACAG
ACTGGGGCACGGGAGAATCAGGGTGGAGGAGGGCTGTGATTTAGGTTTACCATGAAACCGGAGTCATCACCGGTGGGAGAGGCTGGTCA
GAGTTCCTCTTTTTTCCTCTTCTTTCCCTAGGACCCAGGCCTGATCCGGTGGGGAGGCGAATGATTGGAAAATGCTGCCTTCTAGAGTC
CCTAGCGAGAGCCACACCCCAGGCCTCAGCCATCAATGACAACCTTTCTGTTCTCACTGAAAGCAATTAAAGATCTGTAAGATGAGTCA
GTGTTACCCATTCTAAGCTGCTGACTGCCTTGCTCCCATCCCTGGGACCCACATCGTGGAAGGAAAGAATGGACTGATGCCCTTGGACC
CCCACGTGTGTGCTGTGGCACACACACACACGCATGCACACAAAAATAAGTAATGTAGTTCAAAATTTAGAATAAATCAGGCGTGGTGG
TGCACACCTTTAATCCCAGTACTCGGGAGGCAGAAGCAAGTAGATCTCTGTGAGTTTGAGACCAGCCTGCTCTACAGAGTGAGTTTCAA
GCCAGCCATAGATAGCTACATAGCCAGATCTTGTGTTAGCCTCCTCCTTCTACCAACAGAAATTAAGATCACAAATAAAGAGGAAAGAG
GGTGGAAGAGACAACTCAGTGGCTATGAACATTTGCTGTTCTTGCAGAGAAACCAGGATTGAGTCCCGGCACCCACATAGTGGGTAAAC
ATTATCTCTAGCTCAAATTCCAGAGGACCTGGCTTCTGAGAGCACGGTACACATGGGGtGTTCTCTCTCTCTCTCTCTCTCTCACACAC
ACACACACACACACACACACACACACACACACACGCAGGAAAAACAGAAAAGCACATAAAATAAAAACAAATATATAAAAATAGAATCC
AAGGGGATTCAACGATGTCATGAGTCCACAGAAATCCTATCCTCCCCTTTTTCGGGGTGTTACACGGGCGGATCAAACCCAGGGNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNTGACTGCTCTTCCAAGGT
CTTGAGTTCAATCCCAGCAACCACATGGTGGCTCACAACCATCCGTAATGAGATCTGACCCCCTCTTCTCGTGTGTCCGAAGACACCTA
CAGTGTACTTACATATAATAAATAAATAAATCTTAAAAAAAAAAGAAAGAAAGAAAGAAAGAAAGAGAGAGAGAAAGAAAGAAAGAGGT
GAGGGCTGGCTCTGGTCGCGCACACCTTTAATCCCAGCACTTGGGAGGCAGAGGCAGGTGGATCTCTGTGAGTCTGAGGCCAGCTTGGT
CTACAGAGTGAGTTCCAGGACAGCCAGGGCTACACAGAGAAACCCTGTCTTGAAAGAAAGAAAGAAAGAAAGAAAGAAAGAAAGAAAGA
AAGAAAGAAAGAAAGAAAGAAAGAAAGAAAGAAAGGAAGGAAGGAAGGAAGGAAGAGGTGAATAGGATAAAATGAGGTCCAATAGGTTG
GGGGCATGGCTCAGTGTTAGAGGGCTTGCCTAGCAAGTGAGAGGCCTGGGTTCAACCTCTCGCACTGAAGAGGGATGGTAGGTAGCTAG
GTAGGTAGGTAGGTAGGTATCTAGATAGATAGATGATAAATAGGTAGATGGATGGTAGATATATAGATAATAGATGTGGGATAGATAGA
TGATAGAGATATACATAGATGATAGATTATAGATAGATAGATAGATAGATAGATAGATAGATAGATAGATAGATAGACAGACAAATGGT
AGGTATATAGATGATAGAAAGATGAGGGATAAATAGATGGTAGATAAGTAGATGATAGTAGGTACATGAGAGGGAGAGAGAGAGAGAGC
GAGAGAGAGCGAGAAAGGATATAATAAGGTCATTACGCTAATTTGATCCAAAGACAGATTGGGGTTCTTATAAGACAACTGATCAAGCA
GGCCAGATATGGGGGGAACTGATCTGTAATTCTAGCACCTGGGGTGGGAGATGGAGGCAGGAGGAAGGGGAGGTCGAGGGTATCCGCAG
ATACTTAGTGAGTTTGAGGCTATCCCCAGCCACGTGAGACTCTGGCAAATCCTTCCGCACTAACAACAAAAATTCTGAAAGGCAAACCT
CCCTTAACTCATAAAGCATGAATTGGGGCGTATGCGGGCCTTCAGAGCCATGTGGTGTGTTTGCGAACAGACGACAGGAAACACTCTTG
ACAGGACTCAGACCCAGAGACGAAGGCACAGCAGAGTCACCATCTGGACAGGCGTTGGGGCTTGCTGGGCCGCTCACAGGAAGCAGCGC
ACACCAGAGCCATCCGCCTAGGAGCTCGGCCACAGTTTCCTGCAGAGTGCGCCCTGACACACCCGGCCAAAGCAAGCACTGGCCTGTGG
ACAGAAAGCAGACACAGGACGGAGCCCGTTCCGGGGTCAGCTCACTGTACTTGAAGATGTGAGCCCAAAGAAGTGGGGGGTGCCACGGC
GTCTTTTGCAGATGTTACTATGCCAGTTGGTGTTAGACACTTAGATATATTTTTAAGAAGAGCAGTCGGTACTCTTAACTACTGAACCA
TCTCTCCAGCCCCACAATGACATTCTTCAAAGAGCAGAGCTTAGAGGCACAGGCACTTGTCACCAAGCAGCAACATGAGTCAGATCCCT
GAAAATCCACAGGCAGAAGGAGAGAGCTGACTCCTGTCCGTTGACCTTCATGTGAATACAGTGGTGTGTGGTGGTGTGTGTGTGTGTGT
GTGTGTGTGTGTGTGTGTGTGTGCATTTATGCCATGTGTGTCAAGAGTGTGAGGAGGACCCAAGAGAGTTCCTAGACTTAAAGCTGGTT
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNN

| | |
|---|---|
| MOUSE SEQUENCE - mRNA | (SEQ ID NO: 2) |

GAGCTAAGCCGTGTTGAACAAAGGAGGTCGGGCACAGCTATCCAAGCTCCCGGGGCCACCGGGCCGCCCTCCGCCACCATGACCGCCAA
CGGCACGGCACAGGCTGTGCAGATTCAGTTCGGGCTCATCAGCTGCGGCAACAAGTACCTGACAGCCGAGGCGTTCGGGTTCAAGGTGA
ACGCATCCGCTAGTAGCTTGAAAAAGAAGCAGATCTGGACGCTGCAGCAACCTCCCGATGAGGCGCGCAGCGCGGCCGTGTGTCTGCGC
ACGCACCTCGGTCGCTACCTGGCCGCCGACAACGACCGCAACGTGACCTGCGAGCGCGACGTGCCCGACGGCGACTGCCGCTTTCTCGT
CGTGGCGCACGACGACGGCCGCTGGTCGCTGCAGTCCGACCCTCACCGGCGCTACTTTGGCCGCACCCAGGACCGCCTGTCCTGCTTCG
CGCAGAGCGTGTCGCCGGCCGAGAAGTGGAGCGTGCACATCGCCATGCACCCGCAGGTTAACATCTACAGCGTTACCCGCAAGCGCTAC
GCGCATCTGAGCGCGCGCCCGGCCGACGAGATCCCGGTAGACCGCGACGTGCCTTGGCGCGTCGACTCGCTCATCACCTTCGCCTTCCA
GCACCAACGCTACAGTGTGCAGACGTCCGACCACCGCTTCCTGCCCCACGACGGGCGCCTTGTGGCACCCCCCCAGCCCGCCACGGGCT
TCACGCTGCAGTTCCGCTCCGGCAAGGTGGCCTTTCGCGACTGCGAAGGTCCCTACCTGGCTCCGTCCGGGCCCAGCGGCACCCTCAAG
GCTGGCAAGGCCACCAACGTGGGCAAAGATGAGCTCTTCGCCCTCGAACAGAGCTGCGCTGAGGTGGTGCTGCAGGCGGCCAACGAGCG
GAACGTGTCCACGCGCCAGGGAATGGACCTGTCAGCCAATCAGGATGAAGAGACCGATCAGGAGACCTTCCAGCTGGAGATCGACCGCG
ACACAAGAAACTGTGCCTTTCGCACCCACACGCGCAAGTACTGGACACTGACGGCCACCGCACGTGTGCAATCCACTGCGTCCACCAAG
AACGCCAGCTGCTACTTTGACATCGAGTGGTGTGACCGCCGGATCACTCTGAGAGCCTCCAACGGCAAGTTTGTGACCGCCAAGAAAAA
TGGCCACCTGGCCGCCTCGGTGGAGACAGCACGGGACTCGGAACTCTTCCTCATGAAGCTGATTAACCCCCCCATCATTGCGTTCCGGC
GGGAACACGGGTTCATTGCGTGCCGCAAGCTCACGCCCACTCTGGATGCCAACCGTTCCAGTTACGATGTCTTCCAGTTGGAATTCAAT
CACGGCGCCTACAACATCAAAGACTCCACGGGCAAGTACTCGACCCTGGGTAGTGATTCCTCGGTCACCAGCAGCAGCGACACCCCTGT
CGATTTCTTCCTTGAGTTCTGTGACTACAATAAGGTGGCTCTCAAGGTGCCCCGCCGCTACCTGAAGGGGGACCACGCTGGGGTCCTGA
AGGCCTGCCCGGAGACTATCGACCCCGCCTCACTCTGGGAGTACTAGGGCCACCTGCCTCTGCAGCCGCTCTCGTCAGTCCTCCTGTTA
TCCTTACTCATCGGGTGGCCTGCAGCAGGTGGCAAACCCCTTGCCTTTCAAACTGGAAACCCAAGAGAAAACGGTGCCCTTGCTGTCAC
CCTCTGTGGACCCCTTTTCCCTAACTCACTGCTCCCCATGGGTCGGTGGCTGCAGACTGTCCCCAGGAGGACTCTGCTTCCCTCTGTCC
CCTTCTTTCCATGGGGAACTCTGGCACCTTTCTTCTGACCTCAGTCAACTCTGAGCCTTATTTCCCCCCAGGAAGTGGCCTAGGAGAAG
CTACAGGGCCTAGGGACTTACCCTGAGCTTGTAACTGGAAGACCCCGTCCCTATCCCCGCTCCCGCCCCCACCCCACCCCACCCCTGCT
CTGGCCCCAGCCTCTGGAGGCCAGCCTTTTGGCGGGACTGAAGCCGGGCATGGCCAACCTTGCCCACAAGTGTTTTTCTGGATCTTGGC
TGGAAGGCAGTCTGTCCCATCCTGCAGTGTTTGGGCCTGGCTCTTTGACTCAAAGCTAGCTAGGTGGCACTCCGTGTCGCTCCTGCACA
TTCTGGAAGGGCCGGGCCTCTCACCCACCTCATTCCTTTTCCCCCTGGCCTGACTGGAAGCAGAAAAATGACCAAATCACTATTTTTTT
TTTTTCTTTAAGGAAATGTTACTGTTGAAACGCCCTAGGCAAGCCTGCCCTGTTGGTTGTAGTCGTCAGTGGTCTTGGCGGGAGATGCT
TGGCTCCTGTCCCTGCCTCCCCAGCGGTTCCCTCCCTCCCTCCTGCCTGACCACCCCAGCTCTGGCTCTGTGATTGGTGCTCCACGTCT
CCAGACACCTCGGGGCTCCTGGGCGGAGAAAGCCGATGTGCCCCTCCCTGGGAGCCCTGAGTAAACCTCAGGGGGCCCTTTCCCAATCA
CCCCTCCACCGACCCCTCAACACCATGCATCTCACTCTGGGTGTACTCGCTCACATTTATTTTTTTGTAACTGTCATTTCTATAACTCT
GAAGACCCATGATAGTAAGCTTTGAACTGGAAAATAAAGTAAAATCAAGTCTG

| | |
|---|---|
| MOUSE SEQUENCE - CODING | (SEQ ID NO: 3) |

ATGACCGCCAACGGCACGGCAGAGGCTGTGCAGATTCAGTTCGGGCTCATCAGCTGCGGCAACAAGTACCTGACAGCCGAGGCGTTCGG
GTTCAAGGTGAACGCATCCGCTAGTAGCTTGAAAAAGAAGCAGATCTGGACGCTGGAGCAACCTCCCGATGAGGCGGGCAGCGCGGCCG
TGTGTCTGCGCACGCACCTGGGTCGCTACCTGGCCGCCGACAAGCACGCCAACGTGACCTGCGAGCGCGAGGTGCCCGACGGCGACTGC
CGCTTTCTCGTCGTGGCGCACGACGACGGCCGCTGGTCGCTGCAGTCCGACGCTCACCGGCGCTACTTTGGCGGCACCGAGGACCGCCT
GTCCTGCTTCGCGCAGAGCGTGTCGCCGGCCGAGAAGTGGAGCGTGCACATCGCCATGCACCCGCAGGTTAACATCTACAGCGTTACCC
GCAAGCCCTACGCGCATCTGAGCGCGCGGCCGGCCGACGAGATCGCGGTAGACCGCGACGTGCCTTGGGGCGTCGACTCGCTCATCACC
TTGGCCTTCCAGGACCAACGCTACAGTGTGCACACGTCCGACCACCGCTTCCTGCGCCACGACGGGCGCCTTGTGGCACGGCCGGACCC
CGCCACGGGCTTCACGCTGGAGTTCCGCTCCGGCAAGGTCGCCTTTCGCGACTGCGAAGGTCGCTACCTGGCTCCGTCCGGGCCCAGCG
GCACCCTCAAGGCTGGCAAGGCCACCAAGGTGGGCAAAGATGAGCTCTTCGCCCTGGAACAGAGCTGCGCTGAGGTGGTGCTGCAGGCG
GCCAACGAGGGGAACGTGTCCACGCGCCAGGGAATGGACCTGTCAGCCAATCAGGATGAAGAGACCGATCAGGAGACCTTCCAGCTGGA
GATCGACCGCGACACAAGAAAGTGTGCCTTTCGCACCCACACGGGCAAGTACTGGACACTGACGGCGACCGGAGGTGTGCAATCCACTG
CGTCCACCAAGAACGCCAGCTGCTACTTTGACATCGAGTGGTGTGACCGCCGGATCACTCTGAGAGCCTCCAACGGCAAGTTTGTGACC
GCCAAGAAAAATGGCCACGTGGCCGCCTCGGTGGAGACAGCAGGGGACTCGGAACTCTTCCTCATGAAGCTGATTAACCGCCCCATCAT
TGCGTTCCGGGGGGAACACGGGTTCATTGCGTGCCGCAAGGTCACGGGCACTCTGGATGCCAACCGTTCCAGTTACGATGTCTTCCAGT
TGGAATTCAATGACGGCGCCTACAACATCAAAGACTCCACGGGCAAGTACTGGACGGTGGGTAGTGATTCCTCGGTCACCAGCAGCAGC
GACACCCCTGTGGATTTCTTCCTTGAGTTCTGTGACTACAATAAGGTGGCTCTCAAGGTGGGCGGCCGCTACCTGAAGGGGGACCACGC
TGGGGTCCTGAAGGCCTGCGCGGAGACTATCGACCCCGCCTCACTCTGGGAGTACTAG

| | |
|---|---|
| HUMAN SEQUENCE - GENOMIC | (SEQ ID NO: 4) |

TTGCATTTGAAGCTACCATGACTGCAAGAGGGAGTTCCCGAAGGGGTCTGGGAATGGCCCAGGGAAATAGGGGTGAGGTGAGACTCCAC
TGCCCCGACAGTGCCCATGTATGAATTAGAAAAAGTGGGCCAGGCGTGGTGGCTCACACCTGTAATCCCAGCATTTTGGGAGGCTGAGT
GGGCAGATCATGAGGTCAGGAGTTCGAAACCAGCCTGGCCAACATGGTGAAACCCCATCTCTACTAAAAATACAAAAATTAGCTGGGCT
TGGTGGCACACATCTGTAGTCCCAGCTACTCAGGAGGCTGAGGCAGAAGAATCGCTTGAACCTGGGAGGTGGAGGTTGCAGTGAGCCGA
GGTCATGCCACTGCACTCCAGCCTGGGTGACAGAGTGAGACTTTGTCTCAAAAAAAAAAAAAAAAAAAAAAAGAAAAGAAAGAAAGAAA
AAGAAAAAAAGTGGGCCAGGTGTGGTGGCTCATACCTGTAATCCCAGCACTTTGGGAGGCTGAGGTGGGAGGATTGCTTACAGCCAGGA
GTTTGAGACCAGGTTGGACAACATGGTGAGACCTTGTCTTTACAAAAAAATACAAAAACCTATCTGGGCATGGTGTTGCATTCCTTTAG
TCCCAGCTACTTAGGAGGTTGAGGTGGAAGGATCACTTGAGTTAAGGGAGGAGACCACCCCTCATATTGTCTTATGCCCAATTTCTGCC
TCCAAAGAAAGAAAAAGTAAAAACTAAAAGGCAGAAATGAAAACCACAGGCAGACAGCCCAGCGCCACACCCTGGGCCTCGTAGTTAAA
GATCGACCCCTGATCTAATCGGTGATGTTATCTATAGACTACAGACATTGTATAGAAATGCACTGTGAAAATCCCTATCTGGTTTTGTT
CTGATCTAATTACCGGTGCATGCAGCCCCCAGTCACGTACCCCCTGCTTGCTCAATCACGACCCTCTCACGTGCACCCCCTTAGAGTTG
TGAGCCCTTAAAAGGGACAGGAATTGCTCACTCGGGGAGCTTGGCTCTTGAGACAGGAGTCTTGCTGATGCCCCTGGCCAAATAAACCC
CTTCCTTCTTTAACTCGGTGTCTGAGTTTTGTCTGCGGCTCATCCTGCTACAGAGTCTAGGAGGCAGAGGTTGCAGTAAGCCAAATTCA
CGCCACTGCACTCCAGCCTGGGTGACAGAGCAAGACCCCACCAAAAAAAGAAAAGAGGCCAGGCGCAGTGGCTCACGCCCAGCTAATTT
TTGTACTTTTAGTAGAGACGGGGTTTCACCATGTTGGCCAGGCTGATCTCAAACTCCTGTCCTCAGGTGATCCGCCCACCTTGGCCTCC
CAAAGTGCTGCGATAACAGGAGTGGAGGCTCAACTTTTTAAAGAAGAAAAGGACGAATCAGGACAGGAGACAATTACAAGCTCTGTTCA
TTCGGAATTCTCATTGGCTTACAGAAATAACTTTGGCTAGTGATTGGTTATATGTTGTAGACAGACCCACAGGGTGGATGGCGTCTGTG
GCCACTTGGGCATTAGCTGGTCCAGAGCCCAGAGTCCATGTAGCAAGTAGCTTCAGGACGTAATTATTTAGCTCAATAGAAAGTGAGAT
GTGACTGCTGTTACTTTTTTTTTTTTTTGAGACGGAGTTTCACTCTTGTTGCCCAAGCTGGAGTGCAATGGCGCGATCTCGGCTCACTG
CAACCTCTGTCTCCCAGGTTCAAGTGATTCTCCTGCCTCAGCCTCCCGAGTAGCTGGGATTACAGGCGTGTGCCACCAAGCCCGGCTAA
TTTTTGTATTTTTAGTAGAGACAGGGTTTCACTATGTTAGCCAGGATGGTCTCGAACTCCTGACCTTGTCATCCACCGGCCTCGGCCTC
TCAAGGTGCTAGGATTACAGGCATGAGCCACCACGCCCGGCCTTTTAACTGCTGTTACTTTTTTTTTTTTAATCATTTTTATTCTGCTA
GTTCCATAAAAGCAATGTAAACACCAGCATTCTATACATCTTGCTGGTGAACTCACATAATGCTTAGTTCCCTGATCCTTTGACCTCCT
TGTCTTCTCCAGTTATTTTCTGTTTGGACCACTGGCCACAGGAATGGAGTGGGGTTGGGGCTTAGGAGAGAGCAATGTGGCTTTTTGGT
ATGACTTGTTTCATTGACTGCTGTTACATTTTAAATGCCTTTCTGGGCCTGATAATTTAAGGGGGCTGGCATTTCTCACATCAAAGGGC
AAAGGGTTTTTTTGTTTGTTTCTCACTATTGCTCAAAAGCCACATGTGTCAAGGACAGCTCTTGTTAACGGGCAGAGAAGCTTATAGAA
CCTTTCCAGGGCTGGCGCGGTTGTTCATGTCTGGGGGACAAGAGCGAAACTCCATCTCAAAAAGAAAAAAAAAAAAAAGAACATTTCCA
GGCCAGGCACAGTGGCTCACACCTGTAAGTCCCAGCACTTTGGGAGATTGAGCTGGGAGGATCACTTGAGGCCAGGAGTTTGAGACCAG
CATAGGCCACATGGCAAGAACCCAGTTTCTACAAAAACATTTTTTTTTTAAATTAGCTGGGCTCGGTGGTGCACACTTGTGGTCCCAGC
TACTCGGGAGGCTGAGGTGGGAGAATCGCTTGAGCCTGGGAGGCGGAGGTTGCAGTGAGCTGTGATCGCACCACTGCATTTCAGCCTGG
GCAACAGAGGGAGACTCTGTCTCAAAAAAAATCAAATAAAGAAAAAAGAACATTTCCATTATTGCAGAATGTTCTATTGGACGGCGCTG
GGCCAGATGCCTGCCCCAAGCCCTGGGACACCCAAACCTGGTGAAAGTGCCAAGCTCCCATCTGGGGGTGCTCACGGAAGGGGCCGGGA
GCTGGGATTACAAGCGTGGAGGCAGGCGCCACCCCAGAGGAGGTGGGTGATGTCTGAGCCAGAGTCTTTGGGATGAGCAGGGCTTTGGC
AGGCAGCTTTGTTGGGCAGGCAACAGGCACAGCAGGTGCAATGGCATCGAGGTTGGAGAGGTGGACTGGCGGGGAGAAGAGAAGGGAGG
GTGGCAGGAGAGATGGGCAGAGGCCCCCCAGGAGCCCAGAGCCTTCAGGGCTTTGGGCCCTCTTGGGGCACTGGGGAGCCACGGGAGAA
GTGTGGGGTGGAGAGGGGCGCCCTGGATTTGACCACCTTCAGGAACCTACCTTGGCTGCCCGGGTGGGTGGGATGGAGGTGATGAAGGT
GGAGGTCGTAGCATTGTCACTGAGAGCGATGCTTATTCTGATTTTTGCCCCCGTGAGCCTCAGAAATCGGTCAGAACAGTGCTCGGGCT
GGGCGTGGTGGCTCACGCCTGGAATCCCAGCACTTTGGGAGGCCGAGGCGGGCGGATCACCTGAGGTCAGGAGTTTGAGACCAGCCTGG
CCAACATGGCAAAACCCCGTCTCTACTAAAAACACAAAAATTAGCTGGGTGTAGTGGTGCATGCCTGTAATCCAGCTACTCCAGAGGCT
GAGGCCTCCCTGGGAGGCGGAGTGCAGTGAGCCAAGATTGTACCACCGCCCCTCCAGCCTGGGTAACAGAGTGAGACTCTGTCTCAAAA
CAAACAAAAAAGAACAAACCAAAACCAGTGCTCCAATTCACCTTGCTGAATTGGAGAAGAACAGGTGCCTGGGTCCTCCCGGCTGACGG
TGACGTCACTATGAGCCACCACGCCTGGCCCGGTGGGTGTTTACAGCAGAGGTGGTTCAGGGAGGAGTGAGTGGCAGATGGTGCAGGCT
CAGGGAGGAGTGAGTGGCAGGTGGGGCCTGAGGCCAGAGGCAGGAGGACCGTTCAAGACTGAGGTGTCTTGGGCACTGGTGCCGGGGCC
GGGGAGAGCCCTGGCATGCCGGGCCCCGGCCCTCAGTGAAAGCAGGATGAGGTGGCGGCCAAGCAGGGAGGCCAAGGCCTGGCAGCAGC
CGGCAGCCCCTCTCCTGGCAGGGACATCTTGGCAGGAGCCGGGCTCTGCCCAGGGCGCTAATGTCCTCTTAATTAGCCAGGCACTTGCC
AAGCACTTGCCGCTGCGGCCTCACTTTGCAGAACGCACTTGGGTTTGGAGCAGGACCAGCATCCCCCACCCGGTGAGTAGGGGGGATGG
CCAGGTGAGTACCATGGCCAGAAGGTGCGGCCTCTGAGTCCCCACTGAAGCCAATCCAGGCCCCGACCTCTGGTCATCATCAACAGAAG
AGTGACAGAAACAGAAAAGATATCAAGGAACACCTTTTCAGCGCCAGATTCCAGGTCCCCCACCTATTTTCCTTTGCTCCTCCCTTGCC
TGGCTCTGCCCCCTTGAACTCTTATTCACCCAGTGTAGGCATCACCTCCTCCTGAAGGCCCTCCTGGACTGCACAGTTAACTCTGGAGC
CTCCCCTGCACAGGGCAGCTCATCTATGGATCCACAGGTGAGTTCCTGTCTTTCAACGGTTACAGTTTACGTCCTCGAGCAGTCGTGGC
GAGGTCACACCTCCACCCACATCCCTCTTTGCAAATCCCATGGGAACTATGAGCCTGGGTCCCCCAAACTGCATGTTCCCCACTTAAAG
CTGGGCCAGATCCTGTTTGCTGTATCCAGGCCTCAGTTTCCCTGCTTGTAACCAGAAGTCCATGCAGCCAGAAATTGTCAGGGAGCTGG
GCACGGTGGCTCATGCCTGTAATCCCACCACTTTCACAGCCTGAGGCAGGAGGTTCACTTGTGATCACGAGTTTGGGACCCCAGCCTGC
GCAATACAATGAGATCCCTGCCTCTACCAAAAAATTAAAAACAATTAGCCAGGCCTGGAGGCATACACTTGTAGTCCTAGCTATTCAAG
GGAGGCTGAGCTGGGACCATCACTTGAGTCCAGGAGTTTGTGGCTGGGGTGAGCTCTGATTGTACCACTGCACTCCAGCCTGGGTAAGA
CAATGAGACCTTGTTTTATTTTATTTTATTTTTATTTATTTTGAGATGGAGTCTCACTCTGTCACCCAGGCTGCAGTGCAGTGCTGCAA
TGTCGGTTCACTCCAACCTCTGCTTCCCGGCTTCAAGAGATTCTCCCCCCTCAGCCTCCCAAGTAGCTGGGATTACAGGCGTGTGCCAC
CACGCCTGGCTAATTTTTTGTGTTTTTAGTAGACACAGGGTTTCACCATGTTTGCCAGGCTGCACTTGAACTCCTGACCTTAGGTGATC
CATGCCCCCTTGGCCTCCTAAATTGCTGGGATTATACGCATGAGCCACCCGGCCTGGCCGAGACCCTGTTTTAAAAAAATTAATCAGGT
CCAGTGCGGTGGCTCACGCCTGTAATCCCAGCACTTTGGGAGGCCGAGGACGGCAGATCACCTCAGATCGGGAGTTCGAGACCAGCCTG
ACCAACATGCAGAAACCCCGTCTCTACTAAAAATACAAAATTAGCAGGGCGTGGTGGCGCACACCTGTAATCCCAGCTACTTGGGAGGC
TGAGGCAGGAGAATCACTTGGACCTGGGAAGCAGAGCTTGTGCTCAGCCGAGATTGCACCATTCCACTCCAGCCTGGGCAAAAAGAGTG
AAACTCCATCTCAAATAATAATAATAATAATAATAATAATAATAATAATAATAATTGGTAGAGATGTTGGTAGAGGCAGGACAGTGGGA
GTGACAGCCTGGTCCTGTCCTCCCGGGCTCTGGCTGACCATGCTGTAGTCTGGCTCAGGGCCCTGGAGGGCACAGCTGTCCTGTCCCCC
TGTGGTCAGCCGGGAAGCCAGGCCTGGCAGGACTCCTGCCCTAGAATCAGCTCCTCCCACTCCCCAGGGTCCCATGCAGGGCGCTGGCC
GTACCAGGCCTCCTCGCGGGTGCCACAGAGCCACCGGCTGCACAGCAACAGAGACGCCATTCACAGGCTCTCGGCCACGAGGCCGCTCA
TTTACAGAGGGCTGAGCCGCTCACATCTGTGCGTTTGTAACATGGACAGCATTTCTCGTCCCATACAGACGCCTGGGCCAGCCACATTT
ATCTCCCCTTCAGAGAAGGGAAACGAGGCTCGGGAGGGAATGAGGGATTGGCTTGGGGTCACACAACTTTAATGATGACCCTGGGCAGG
AAGCTCCCAGCATGGCAACAGAGACTCAGGGCAGGCTGCGCATGGTGGCTCACACTTGTCATCCCAGCCCTTTGGGAGGCAGAGGTGCA
AGGATCACTCGAGCCCAGGAGCTCAAGGCCAGCCTGGGGAATATAGTGAGAACCCATCTCTAAAAAAAAAAAAAATAGGCCGGTCGCGG
TGACTCACGCCTGTAATCCCAGCACTTTGGGAGGCTGAGGCGGGTGGATCACGAGGTCAGGAGATTGAGACCATCCTGGCTAACACGGT
GAAACCCCGTCTCTACTAAAAATACAAAAAATAGGCCGGGCGTGGTGGCTCATGCCTGTAATCCCAGCACTTTGGGAGGCTGAGGCCGG
CGGATCACGAGGTCAGGAGTTCGAGATCAGCCTGACAAACATGACGAAACCCTGTCTCTACTAAAAATACAAAAATTAGCCGGGCGTGG
TGACACGCGCCTGTACTCCCAGCTACTCAGGAGGCTGAGGCAGGACAATCGCTTGAACCATCGAGGTGGAGGTTGCGGTGAGCCGAGAT
CGCACTACTGCACTCCAGTCTGGGAGACAGAGTGAGACTCCGTCTCAAAAAAAAAAAAAAAAATTAGCCGCGCGTGGTGGCGGGCGCCT
GTAGTCCCAGCTACTCGGGAGGCTGAGGTAGCAGAATGCCGTCAACCCGGGAGGCGGAGCTTGCAGTGAGCCGAGATCGCGCCACTGCA
CTAAAGCCTGGGCAACAGAGCGAGACTCTATCTCAAAAAAAAAAAAAAAAATTTAGCCGGGCATGGTGGTGCATGTCTGTACCCCCAGC
AATTTGGGAAGCTGAGGTGGGAGCATTGCTTAAGCCCAGGAGTTTGAGGCGGGAGTGAGCTGTGATGGCACCACTGCACTCCAGCCTGG
GTGACAGAGTGAGACCCCGTCTCTCCCTATCCCCCGCAAAAAAAAAAAAACAACTCAGGGCTTTCCCACCCCTGTCCTTGCACAGATGA
AGAACCGAAGCTTCTAGAAGGGGTATATTTTGCCCCCAGGCCCCAAATCCTGGTCTTTGGACTACAGTCAAGACCTAGCACAGGGCTCA
GCCCTAAAAAAAAGCTGTTGAGGAGGGTGTCAGGCTGCAACGTTGCGGTGAGAAGGGGGTCCCCAGGGAGGGCGCAGCAGGAAGCCCCA
GGGAAGTGCCTGGGACAGGGAGGTTGTGCAGCCAGAAGGAGCAGCCCGCAGCCTTTGGTTGTTGACTCCTGCTTTGTAAGTGGCAGTCT
GGTTGGGTAGGACAGGGTCCGACTCCTCACTCAGGGAACTGAGTCCAGGGTGAGCTCAGCAGCCCTTCCTGGTGGCCTCACTTTCCCCT
GCAGAGCCTGCTGCATGGTGTCCAGTCGCCAGCCTGGCAGGAGCTCAGGACTGGCCTCACCTCGTGCCACCCCCTCGTACCAAGGTGGC
TCAGATGGCACCTGGGTTCCCGAAGGGCCCAGGAACACAGCGTCCATGTCCCCATCCTTCCCCGGAGGGACTTGGGGTCGCGCCTGCAC
GAAGGATCATGTGACTTGTTCAAGGGCAGCTTGTCCTCCCCCGGACACAGAGGTGCCCATCGTAAGCGAGATGCAGGCAAGGTGAGGAC
AGGGCATGGTGCCGAGCAGGAATGATTTTCGAAAATGCTACTCTGTGGTCGGGCACGGTGGCTCACTCCTGTAATCCCAGTACTTTGGG
AGGCCCAGGCGGGCAGATCATGAGGTCAGGAGTTCAAGACCAGCCTGGCCAACATGGTGAAACCCCTTCTCTACTGAAAATACAAAAAA
CTACCCGGGCGTCCTGGTGGATGCCTGTAATCCCAGCTACTCAGGAGGCTGAGGCAGGAGAATCGCTTGAACCCTGGAGGTGAAGGTTG
CAATGAGCCGAGATTGCACCACTGCACTCCGACCTGGGCGACAGAGAAAGACTCCGTCTGAAAAACAAACAAACAAAACCCCGAGATTC
TCTTTTCCCCCGTCCGGAGCTCTATGGCCATCTGGAGCTCGTTCTGTCCACAAGGACACATTTCCTGGCAGCAGCTGTGGACCAGGGCT
CACTCACTCACATCTGTACCCAATCTAGAGCAGGACAAACACCTATCACCTGCACTGGCAATGGACAGAGGACACTGCCAGCCCCATCA
CCAGAGGCATTCAAGCCAACGCATTTTCTATCGTCTATTTATCTATCTATCTATCTATCTATCTATCTATCTATCTATCTATCTAGATG
AGATCTTGCTATGTTGCCCAGGTTGAACTCCTGGCCTCAAGCGATCCTCCTGCTTCAGCCTCCCAAAGTGTTGGGATTTCAGGTGTGAA
CCACTGTACCTGGCTGGAGTGCAGTGGCACTGTCATAGCTCACTGCAGCCTCCACCTCCTGGGCTCAAGGAATCCTCTTTCCTCAGCCT
CCTGAGTAGCTGGGACCACAGGCATGCACCATCACACCCAGCTAAACCTTCATTTTTTCCATAGTATATGCCTCAGGGCAGAGCAAAGA
AGCACAAAAACATGTTGGCTTGCGGTTGAGGGCTGATGGGAGGGGGTTCTGGCCAAAGCCCAAAATTAACAGCCACAACGTCAGTGTCT
GTGGGAGGGGTTGCCAGGGGCGTGCGGGTTCTGGGGCTCAAGGCCCTGCCGGTAAACCCATTTGAAGCAGGATGGCAAGAAGGTGACAC
CATCTTCCCCCGCGCACTGAAAGCCCCTGGCTGGGATTGCCTGGCGCAGACACAGGCTCGGACCAGCCCCAGCAATCCCAGTTTATCAG
CGAGCCGGCTGAGGGCCCGGAGTTATCTCAGTGCCCGGCCTGAGACCTTGTGGGCAGCCTGTGGTCATGCTGGCATTCCAGGGGCCTTT
TGGCATGTGGGGAATGTCCAGGAAAAGCCTCAGCCTTCGGTGAGGCGCAGAAAAGGGAAGTGTCCCTAGAGGGGGTGGGTGAGGGCGTG
GGAGGTCGTGTCTGCAGGGAATGTCCCCTTTGCGCGAGGAGGATGGAGGGTTGGGATTCTGAGGATGGGGGCGGGGGCTGTAGCCAGCA
CCATGTCCCTCCTGTGTGACCAGCTCAGAGTCCCATGAAATTGGGGCTTGGGAGGGGAAGGGACACTGGCCTGGGAACCAGAGACCTGG
GCTGGTCTGGCTCACAGTTGCTGCACCTCTGTGATCCGTGTCAAAAAACGAGAACACCAATTCCTGTCCTGCCCACTCACCACCAGGTG
GGACCTGAACCCTGGCATCGCCAGCATTGGGAATGTCCGCCACTGACTCAACCACTCTCCCGGAGACCTATTTGGGCCACCCGAGGCGG
GTGCCTGGGCCACACGGAGGGGTCCTGGTGGTCTTCAGGGCAGCGGCTGTGGGGCTGAAGCCTCAAGGAACCACATCTCTGCATAGGAG
CCCCAGGCTGCAGGGCCTCGGAGACAACCTAGTTGGGCGTGTTGGGGTCTGTGGGTCCCACGTCCTGGCCTCACCGGGTCCCCACCGCG
CTGTCAGCTCCCAGCCTCTTTCCCTCGTCTCCCTCTGGGCTTCTGTAAGGCTATGTGCTCCAAGCCCACCTCCTCCAGGCAGCCCTCAG
ACCCCCACCTTCGCCCAGTACCGATCTGCACCGTGGTCTCTGAAGTCTCCATCGTGACCTCAAACCTCGCTCGTCCTTGCTCCTACCGA
GGCTTCGGGTCGGGGTGTCCGACGTGGGGGACATCCGGGGGGGTTAGGTGGCTGGCGCGGGGAGCCGGGGTTGTGAGGGGTGATGTCCT
CAGGCGGCGGCGCTGCGGGGTGCGGCGAGGACACCGGTGGGGTGAGAGCACCGGCGGGGCAGCAGCGGGGGCCGCAGCGCCGGGTCCCT
CGGCCCGGGGCCCCTCCCCCGCGGAGCCAGGGGCGGGACAGGGGGCCCTGGCCTGGTCGCCCTGACGTCACCTCGCCTATAAAATGTCC
GGGGCGCCGCTAGCTGGGCTTTGTGGAGCGCTGCGCAGGGTGCGTGCGGGCCGCGGCAGCCGAACAAACGAGCAGGCGCGCCGCCGCAG
GGACCCGCCACCCACCTCCCGGGGCCGCGCAGCGGCCTCTCGTCTACTGCCACCATGACCGCCAACGGCACAGCCGACGCGGTCCACAT
CCAGTTCGGCCTCATCAACTGCGGCAACAAGTACCTGACGGCCGAGGCGTTCGGGTTCAAGGTGAACGCGTCCGCCAGCAGCCTGAAGA
AGAAGCAGATCTGCACGCTGGAGCAGCCCCCTCACGAGCCGGGCAGCGCGGCCGTGTGCCTGCGCAGCCACCTGCGCCGCTACCTCGCG
GCGGACAACGACCCCAACGTCACCTGCCACCGCGAGGTCCCCGGTCCCCACTGCCGTTTCCTCATCGTCCCGCACGACCACGCTCGCTG
GTCGCTGCAGTCCGAGGCGCACCCGCGCTACTTCGGCCGCACCGAGGACCCCCTCTCCTCCTTCGCGCAGACGGTGTCCCCCGCCGAGA
AGTCGAGCGTCCACATCGCCATGCACCCTCAGGTCAACATCTACAGCGTCACCCGTAAGCGCTACCCGCACCTCAGCGCCCGGCCGGCC
GACGAGATCGCCGTGGACCGCGACCTGCCCTCCGGCGTCGACTCGCTCATCACCCTCGCCTTCCAGGACCAGCGCTACAGCGTGCAGAC
CGCCGACCACCGCTTCCTGCGCCACGACGCGCGCCTCGTGGCGCGCCCCGACCCGCCCACTGGCTACACGCTGCAGTTCCGCTCCGGCA
AGGTCCCCTTCCCCGACTGCGAGCGCCGTTACCTGGCGCCGTCGGGGCCCAGCGGCACGCTCAAGGCCGGCAAGGCCACCAAGGTCGGC
AAGGACGAGCTCTTTGCTCTGGAGCAGAGCTGCGCCCAGGTCGTGCTGCAGGCGGCCAACGAGAGGAACGTGTCCACGCGCCAGGGTGA
GTGGGGACGCTGCCCCCGCCTCTCCTGGTCCGTGCACAAAGCGCACCCCACCCGCGCCCCTCCAGCCTCCCGCCCTTTCTCGCTCGCGG
CGCCGCTGCGGTCCGGAGCACTGCCCATTGCCCCCCCGCTAGGCACCCGCCCTACCCCGCCTGGAGGGGGCGAGGAGTGGGGCTTTGCC
CATCCTCGCGTGCCGCTGCCCACCTCCCACCCCGCGCTGGGATCATGGGCTCCCCTAGGCCCCCCCGAGTCGCAACCGTACGTGCACCC
TCCTAACCCCCCCCCCCGCCCAATCTTGGCTCTCCCCACGCGCGCTGATCCCTCGGATCAGCTGTCCCAGCTCTTGCGGAGTCCCAGCC
CCTCACCCATTTCCTCGTGCCCCTCCCCCCGCCGGCTGTCCTGGAGCTCGGGGGTCCGAGGCAAGGGTCGCCACCCGCAAGGGCGCGCC
TCCACCCCCACCGGCAGCCTTTCGCGGGCGACATGGCGGAGGTTAGCCAGGCCTTTCATCCCGGCGGGGCGCGCCTCCACCTCCCCGTC
TGCCCGGCCTTCCTCCACCTCCTCCCCCTGCCGGGCGGGCTCCCCCTCCCCTGGTGGGGGGGGGCGCGCGGTGTCAGCCCTTCCCCCCA
GCCCCTCCTCCCGCGTCTGCCCCGCCCTCGAGGCCGCGGCCTTTGTGAGCACGGGGGCGGGTCGCCTCGACTGCCTCCTCCCTCGCCCC
CTTTGTCCTCCTCCATCTCTGCAGATGGGAAAACCAGATGCGGCGGCGCGGGGGAGGGGATCGGCTTTTGCCGTTCACCCCTGCAGACG
AGCCCCCCGCGCCGCCCCCGGCCCCAGGGCTCGGGTCCCGAGGTTCCCCAGGAGGCGGTCTCCCTCCTCGCGCCGCGGCCCGGGAACGG
CGTGGCGCGGATGGCGGCCCTCCAGGCACCCCGCCCTTCGCCCGCCGCGCGCGTCTCCAGGCCGGTGCGCTGAGCTCCGCTCCGCGGGC
GACCGAGGGCGGCTTCAGCGCGAGCCGGGAGACCCCAGGCCAGCTCCCTCGGGAAGCCCCTACCCTCTGGTGAACCCATCCCCTAGGGC
GCTCGCCGGGAACAATTGACTGCAACTCGATCCGTCCCCTCCGGGGCCTCCTGCAGCCACCGCTATTTGCGACCGGCCTGTCCCCCACT
CTTTCCCTTCTTTCTCAGATTCTCCCCGAGGGGCTTTCTCTTCCTTTTGGCGTCCTCTTCCACTCCTCGCGGAGAAGCTGTTCCTTGCT
TCCAGACAGAGGCCCCGCTGGAGGGAGCCGCGTGGGGGGATCTTTTTCCCTGGATAGGAAGTCGGACCCCAGGCCTTGGACATGGCTGG
ACGGGAGCCCAGTTTGTGTCCTCATCCCTCCTCTTGGAAGGAGCCCTCCTGACGGCCCCCCTCTTGGCTGTGGGCTCTGCAGGAGGGGG
AAAGACCCCCCATGGCAGTGCGGGTGAGGGTGGAGGCCTGGGTGGCCTAATGGCCATTTTGTGCCTGAAGTTTGCTACCTTAAGTCCCA
GAGAGGTGAAGCTGCTTGTCATGGGTCACACAGCAAGTGACCACAAGGAAGCAGCATGCAGAAGTGGGTGCATTTGGCTCCAGAAACCC
CGTTTCCTGTTTCCCACAGCGCATGGCGGGCAGAGCTCCTCACCTGATCAGCAGGCATTGAGCCCTACTAAGGGGACCTGCTGAGCCAT
GAGTGAACCAGTGGGGTTCACGTTCTCTTCGGGACATTACGAAGTGTCAAGGAAAAGGCAGCGGGTACAGGTTATTAGATGCCTGAGTC
CTTTCTGAGCAGGACATTTGAGCTGGCCTGAAGCATGAATAGAAGGTGGCAGGTATGGGGGGGGTGCTGTGTGGACCAGCGTTGTAGGC
TGTGGGTGTCTCCTGTGCAAAGGTCTTATGCCAGGAAGGTACAGAGAAAGCCCAGTGTGGCTGGGAGGAAGCTTGTGAGGTCTGGGCCA
CGGCACCTTGGGTGAGAGCTTAGTGCAGGGCTATGGTCCTTGCCTTGGGGTTGCAGCCACAGCCTCCCGTGGGGAAGAAGTCGCAGAAG
TAGGTGGCCTTGGCCCTCCGGGCCAGGAAGTTGCAGAGAGCCTCAGGGTGTTTTTGGCGAGCCTGGGCAGATAACTCCCCTCTCCTGAG
AAGCTTGCTGGGGGCGTCTGGTGTGTGATTCAGGCTGATAGGGTGGGAGCAACCAGAAATTGCAGCCAGGACAGGTCGTCGTGATGGCT
CAGCTTGGAGTTGAAAGGGATGTGGGACGGCGCGGGGCTGGGGGTACTTGGGGGCCGAGGTTGCGGCTCCTCTGGCCTCAGATCTTGGG
GTCCTTATCTCGTTTCTATGTCAGCCAACTCCCGGGGGGCTTTTGGGAACTGGAGTCTGCAGGGGTGGGCGTGGGGGGCGTGTCTGCAG
AGCCCATTTTGGACGGGCCTGCGTGGCAAGGGGGAGGCGTCTGCTTCCGACACACATGCTGGGGAACGCCAGCCAGGAAGGGGAGGATC
CGGACTTAGCTGGCAGGGGGGATGTGAATCATCTCTGCAGGCCTGCACGGGCGGGCCCGGTGCGGGCAGGGTGCTCCCCTTTGAAAAAC
GCTGCGCCCTGCTCTTCAGCCTCAGTTTCCCCATCTGTAAAGTGTGCATTCCAGGCCTCCTTGGGCTGGCCCACACCTGCCCTGGGCAG
GGCCTTTCAGCCCTTCACACCAGAGGCTCCGTCAGCTGAGTGAGTGCTCCTTTGTCCTCCCCCCATGCCCCAGGCTCCTCCCTGCTCCC
CAGGATCCACACCTACCCTGGCCCCGACCCTGGGCCATGCCTCACCACACTTCCTGTCTTCTCTCACATCTCTCACATCTGGGAGTCCT
CTCCCGCCAGCCTGTGCTTGCATCTGGGGATCCATGCCAGGGATACGACCTGTCCTCCCTGCTGGCTTGGGACATGCCCTCTCCCAGCC
ACTCTGAGGAGGGCTGACTGAGGAAGGGCTCGTCAAGCTGGGCTTTGCAGGATGTGTAAGAGTTCTCCCCACGAGGCGCAGGGCATTCT
GAGCCCAGGGAATGGCTTGTATAAGGATGCAGAGGCATTTGAAATGGCCAAGTAGTTGCAGGAATCGACTGGAAACCGGGGTGGTAAGG
TGAAGCCATAGAACATTCCAGGCCCCCTCCCCTAAATGAGATGGAAGGAGTGCCTGTTTTGAACAAGCCAGGGGCATCTGGGGACCGTT
AAGGCCTGGGGGTGGTGATGGGGACTGGAGGGTGTGAGGCAACCAGGGGGTGCCCCTCTGAGCCACCACACACAGAATGGTTCAGAAGG
CCAGGCACACTGGCTCTCGCCTGTAATCCAAGCACTTTGGGAGGCCAAGGAGGGAAGATGGCTTCGGCCAAGGAGGGAAGATGGCTTCA
GCCCAGGAGTTCAAGGCCAGCCTGGAAAACATGGCAAAACCTCTGTCTACAAAAAATACAAAAAATTAGCCAGGCATGGTGATGCACGC
CTCTCGTCCCAGCTACTCAGGAGGCTTAGATGGGAGGATCACTTGAGCCGGGCAGTTTGAGGCTGCAGTCAGCCGAGATCGTGCCACTG
CACTCCAGCCTGGGCAACAGAACGAGACCCTGTCTTAAAAAAATTTTTTTTCGCCTGGTGCGGTGGCTCACGCCTGTAATCCCAGCACT
TTGGGAGTCCAACGTGGGTGGATCACCTGGAGGTCGCGAGTTTGAGACCAGTCTCACCAACATGGAGAAACCCCATCTCTACTAAAAAC
ACAAAAATTAGCCGGGCGTGGTGCCGCATGCCTGTAATCCCAGCTACTCTCGAGGCTGAGGCAGGAGAATCCCTTGAACCCGGGAGGCG
GAAGTTGCAGTGAGCCGAGATCGCACCATTGCACTCCAGCCTGGGCAACAAGAGTGAAACTCCATCTCAAAAAACAAAACAAAACAAAA
AAAACAACCAAAACAAACAAACAAAAAACTGTAATTAAAATAATTAAAAGAAAAAAGTTAAAAAAAAAAAAAAGAGAGGATGGCTCAAA
GGCTGGAAACAGGGAAGGCCACTGTGAGGGGAGGACAGGCAGGGCTAGACCTCTCTGGAAGGAGGAGGGAGAGGTACCCCTGGGCCCGG
CACAGGAGACTCTTAATCTCCTGGCTCCCGGGGGCTTCTGTGCGCCTGTGACTCAGGATTTCTGTGCCTCTGTTGATGAAAAGAAAAAT
CCTGAAGAAAACACTGTTCCCATAGTAACACAGCTCTAATTAGAGACTCCAAGGCCAAGCGAGGGCCTTGGAGCCAGGAGGGGCCCTGC
TCTACTGCGCGGACGACACCCCTTTCCCCTATCCCTCCCTGCTGGGGTGTGGCCTGATGTCTACGTGGCACCAGGCCCCTAAATCCTTT
CAACATCCTGGCGCACCGGAGACGCTGGGCCCTTTTGACAGGTGGGGAAACTGAGGAAGGTTGCACCGAAGGCACTCATCCAAGGGTGC
CTGGCCCCCCGCGGTGGGTGCCTCACTGGCTGGCCTAAACCTTTGCATCAAACCAGTTCCAGGATTGAACGCAACAGGCTGATGCGGAC
TGAGTCGTCGTACAGATGGCGGCAGTTGTGTGGCCCTGGGCCCAGGAATGGCTGGGAAGCTCCCTTTCTTCTCTGGCTTGGGGGATGAG
GTTAGTGTAAATTTCATGGAGTTGCCAGGAGGATTAAGGCCGACGTGCATTCACACGTGGCCCCGTGGCTGCCGCCAGTTAAGCCCTGC
GCAGCCATTGGAAGTCTCATTGAGAAGGAGCCTCCCGGCTTCCTGCATTTGTTCCAGGCGGGCTGGTAAGCGCCCTGCTTATTTGCAAG
GCTGTGTGAGGACGAGCCCTGTAAACCCTAGCACGAGGCTGTGCTAATGTGGTTTCTGTTCCACTGTCTCCCCTTCTCCTTTTGCCCCT
CTTTCTGGGGAACCTCGAGCCCCTGATGCCCTCTGTTTTTTCCTGCTGCTGGGGTGGACCTAGAACCTCTCCCTCCATCCTTTCTGCTG
CTGGGGTGGGCCTGCAGGAGGCTGTCCTCTTACCTGGTCCCTAACTTCCCTCCGATCAGCGGGGCTTCAGCGCAGCCCCAGCGCATTGG
ACAGCTCGTGCTCTGTGGCCGGCACAGCCCAGGGCCCAGGGTGCGGCCCCTGCCTGTAGGTAGACATGCAGGCCCTTCTGAGCAGAATG
AGGGGGCGGTGAGGGCAGCTGTGCGGGTGTGGCTGGCCTCACGCCTCCCTGCAGCCCTGCGGCTTCTGTGCAGTGGGGGTGGGGCGGGC
CAGACCTTTCCAGGGCCTCTTCGCTGGTGGAGGGCTGAGTGAGCAGAGGCCCCAGCCCTCGTGTTCCCTGGGGTCTGCCCTTACGATGG
TCCCGGCACCCTGGGGACCCAGCCCCTGCTCACCTTATCCTCCTCCCGTGCTTGGCTGGGGTGTGGTGGCTGAGCCAGCCCACCCAGTG
CGGTGGATGCTCGTCGGAGGGCAGAGGGTGGGCTACCGGCTTAAGGGGCCCCAGGGAACCTGGGGGGTGGAGCCCAAGGGGTTCCAAGA
AGGGGCGGGGCCAGGAACCCATAGACAAGAGGGTGGGGCAGGGAAAGGGCGTGGCAAGAGGGCCACCCACCTCCGGTCCAGAGAGCCAG
CCAGACCCTTACTTTCTCTTGCTGTGTGACCTTAGGCAAGCACATGCCACCACCGGCCTTAGTCTCCCTCTTTGTGAACTAGGATGAAG
ATCCCCACCTGTGGAGCAGATGTGGGGCTGCATGGTTGGGTCAGGATGGAACAGGATGGGGAGGCCGGGCGTGGTGGCTTACCCCTGTA
ATCCCAGCACTTTGGGAGGCAAAGGTGGGAGGACTGCTTGAGTCCTGGAGTTTGGGCAACATAGCGAGACCACCCCCATCTCTACAAAA
TAATGTTAAAGTTAGCCAGGTCTAGTGGTGAGTGCCTGTGGTCCCACCTACTGGGGAGACTGAGGCAAGAGGATCCTTTGAGCCCAGGA
GGTGGAGGCTGTAGAGAGCCATGCTTGGGCCACTTGCACTCCAGCCTGGGCAACAGAGCAAGACCCTATCTCTAAAAAAAAAATGGAAT
TGAGGATGTGTCCTCTCGGTGTGGGCTCTACCACCCACCAGCCTCCCTCTCCTGTGGGTCCTGCCTGCCACCCACCGCTGAGGTCCCTG
CAGCATCAAGCTGCCCAAACGAAGACACTCTCCAGCTCTGACCCAGGCACCCATACAGAGCCCGCCAGACGCCTCTGCTGCTGCAGTTC
TTAGAATCCAGAGCGCGTGGGGAATGTGAATTTGTGCTGCTGGAGCCAAACATCCCACCTCCAGGTTTTACCTGGAGGAATCCATGTGG
ATATGCAAAGCAATGTAGTTATAATGAATAGCAATTGTGGGCTGGCCGCAGTGGCTCATACCTGTAATCCCAGCACTTTGGGAGGCTGA
GGTGGGTGGATCACCTGAGGTCAGGAGTTTGAGACCGGCCTGGCCAACATAATGAAACTCCGTCTCTACTAAAAATACAAAAAAATTAG
CCGGGCATAGTGGCGGGTGCCTGTAATCCCAGCTACTCAGCAGGCTGAGGCAGGAGAATCACTTGAACCCACCAGGCAGAGGTTTCAGT
GAGGCGAGATCGTGCCATTGCACTCCAGCCCGGGAGACACACCGAGACTCTCTTTCTCAAAAAAAGAATAGCAGCTTTCCTATAGCTTA
GGGGAGCAAAACCCCAAAGCCACCTTAAGTTTCCCCAAGATAGAGATCCCTGGGGTCCTATTCTGAGACACAGTCTTGCTCTGTCGCCC
AGGCTCGAGTGCAGTGGCCGATCTCAGCTCACTGCAGCCTCCGCCTCCTGGGTTCAAGCAATTCTCCTGCGTCAGCCTCCCGACCAGCT
GGGATTACAGGCCCCCGGCACCACCCCCAGCTAATTTTTTTTTTTTTCAGACAGAGTCTCGCATTGTCCCCCAGGCTGGAGTGCAGTGG
CGCGATCTTGGCTCACTGCAAGCTCCACCTCCCGGGTTCACGCCATTCTCCTGCCTCAGCCTCCCGAGTAGCAGGGACTACAGGCGCCC
GCCACCGTGCCCGGCTAATTTTTTTTTTTTCTATTATTAATAGAGACGGGTTTCACCGTGTTAGGATGATCTCGATCTCCTGACCTCGT
GATCCGCCTGCCTCGGCCTCCCAAAGTGCTGGGATTACAGGCATGAGCCACCTTGCCTGGTCCTTTTTTTTGTATTTTTAGTAGAGATG
AGGTTTCACCATGTTGGCCAGGCTGGTCTCAAACTCCTGACCTCATGATCCGCCCGCCTCGGCCTCCCAAAGTGCTGGGATTACACGTG
TGGGCCACTGTGCCCGGCCAGTCCCATTCTACTTTTTAGGGACATGGAATCATGTAAAGATGCCATACAGAAAACAGTATGCAAATCCA
GAGACAGAGGACATCTGTGCATGTTTCTTTCTTTCTTCCTTTTTTTTTTTTTTTTTTTTTTTGAGATGGATTCTTGCTCTGTCGCCCAG
GCTGGACTGCAGTGGTGCAATCTCACCTCACTGCAACCTCTGCCTCCCAGGTTCAAGTGATTCTCCTCCCTCAGACTTCCAAATAGCTG
GGATTACAGGCATGCCCCACCATGCCCAGCTAATTTTTGTATTTTTAGTAGAGGCGGGGTTTCACCATGTTGGCAAGGCTGGTCTTGAA
CTCATGACCTCAGGTGATCCACCCGCCTCAGCTTCCCAAAGTGCTGGGATTACAGGCGTGAGCCGCCGTGGCAACCCGCATGCTTCTTA
TGTATAAGAAAAAAACAGCTAGAAAGTTGTGGGTTCACTGGCTGGGCACATGGTGGCTTGTGTCTGTAACCTCAGCACTTTGGGAGGCT
GAGTTAGGAGGATCATGTGAGGCCGGGAGTTCAAGACCATCCTAGGCAACATAGCCAGACCCTGTCTGTACCAAATAGAAAAAAAAATT
AACTTGATCTGGTGGTGTGTGCCTGTAGTCTCAGCTATTTGGGAGCCTGAGGTGGGAGGATCACTTCAGCCCAGGAAGTCGAGGCTACA
GTGAGCTATGATTGTGCCACTGCACTCCAACCTGGGCAACAGAGCAAGACCCTGAATCAAAAAGAAAGAGATGTATTTGCCAACACAGG
GGTGTTCTGCAGTGGGGTGGGAACGGGACGTGTAGGACTTCATAGTTTCCTTTTTTTTGTTTTAGAGACAGGGTTTAAAAAAAAATTTT
TTTTTTTTTTTTGAGGCGGAATTTCATTCTTGTTGCCCAGGCTGGAGTGATCTCGGCTCACTGCCATCTCCGCCTCCCAGGTTGAAGTG
ATTCTCCTGCCTCAGCCTCACGCAATTTTGTATTTTTAGTAGAGACGGGGTGACTCCATCTTGGTCAGGCTGGTCTCAAACTCCCGACC
TCGGGTGATCTGCCTGCCTTGGCCTCCCAAACTGCTGGGATTACAGGTGTGAGCCACTGTGCCTGGCCCCTTTTTTTTTTTTTTAAGTA
GGGTCTTGCTAAGTTCTCCAGGCTGGTCTTGAACTCCTGGTCTCAAATGATCCTCCTGACTTGGCCTCCCAAAAGGCTCCGATTGCAGG
AGATATGAGCCACCGCACCCGGCCCTTTTATAAGTTTTCTAAAGGAAAGGAACATGTACCATCGTCTTTTATTTTAATTTTTACATACA
AGCAGGAACAATAGCATGAACCCCCAGACACGGCCCCCCAACATGCCCTGTTCCGCTGTGCTGCAGCCCCATTTGTCCTTTTCTATACC
ATTTTTGGTTGTATTTGAAGCAAATCCCTGACATCAGGGCATTTCATCCCAAAATACTTCCATCTGTGTTTCTAAAAAAACGAGGCCAT
TTTCTTATGTAACCACAACGTGATCAACAGTAATCAATGCTTAACATCTAATTCTGGGCCACGTTAAGACCCTCCTGATTGGCTCAAGA
ATGTCTTTTGTGCAGTTGGTTTCTTGATTCTTTTTGTTTTTTTTCAGACAGGCTCTCACTGTGTCACCCAGGCTCCAGTGCAGTGGTGC
AATCACGGCTCACTGCAGCCTCAGCCTCCTGCCTCAGCGATCCTCCTGCCTCAGCCTCCCAACTAGCTGGGACCACAGGTTTGCACCAC
CACTCCCAGCTAATTAAAAAAAGTAATTTGTAGAGACGGGGGTGGGGTGGCGGTCTTGCTATGTTGCCCAGGCTGGTCTCAAACTCCTA
GACTCAAACAATCCTCCTGCCTTGGCCTCCCAAAGTGTTGCGATCACAGGCATGAGCCCCTGTCCCTGGCCCCTATTATTTCTCTAACT
GGCGAAAGTCATGTGGGAACACATGTAGCTTGCCTTGGCCTCTGACCGGCCCTGCCTGCGTTCCTGGGTGCTCTCTGCTGCTTCTCATG
TGTGCCACTGTGGGGACTCGGCCGCCCACCCCACCCCGTGGTGTTACCTTGCGTGTGTAGTTCTGTGAGCTCAGGGCTATGGTCTGCCA
GAACTAGGGGGCGTGGGGCCCCAGTACCAGCCCAAGGCCTCCTCTCTGCAGGTATGGACCTGTCTGCCAATCAGGACGAGGAGACCGAC
CAGGAGACCTTCCAGCTGCAGATCGACCGCGACACCAAAAACTGTGCCTTCCGTACCCACACGCGCAAGTACTGGACGCTGACGGCCAC
CGCGGGCGTGCAGTCCACCGCCTCCACCAAGTCAGTGCCTCGCTCCCACCTCTCACCGCCCCCACCACCTTGCCTGGGCTACCCCGCCT
GACCCTGTCCCGCCATCCCCCAGGAATGCCAGCTGCTACTTTGACATCGAGTGGCGTGACCGGCGCATCACACTGAGGGCGTCCAATGG
CAAGTTTGTGACCTCCAAGAAGAATGGCCACCTGGCCGCCTCGGTGGAGACAGCAGGTAACACTAAAGCCCCAGTTCCCTGGAGCCGTC
CTGGAGTCCTGGAGGGTCTGGCCATGCCGTGGTCACTTGGTAGCCCCAGCCAAGGCCTGCTCTGTGCTGGGCATCCCCCCCGACTGGCC
CCGCACTGTCCTACCCTGGGGCTCACTGCTGTGTGACCCCAGCTCCTGGCCCTCCCTCTCTGGTCACCCCAGCCTCCACCCCACTCCCT
GCCAGGAGGCTCACTGACTCCCCTCTTTCTGCCACAGGGGACTCAGAGCTCTTCCTCATGAAGCTCATCAACCGCCCCATCATCGTGTT
CCGCGGGGAGCATGGCTTCATCGGCTGCCGCAAGGTCACCGGCACCCTGGACGCCAACCGCTCCAGCTATGACGTCTTCCAGCTGGAGT
TCAACGATGGCGCCTACAACATCAAAGGCAGGTTCTCCTGTGGGCAGCTGCTGGGCAGGGAACCCCTCGGTCGGCGCTGGGGTCAGTGC
TGCCGGGAGCGCCCTCTGCATCCACACTGGACCCTGGCTTGGCTCAGGGCCATTCCAGGCCCTAAAGGGACACGTGTCTGATGGCCACC
AGGGGCTCTGGGATGCAAGCAGCCCCTTTCCCTCTTGTCTGTGTGGTTCCGGGGACTTACCTTGCCCACCTGACAGAGAGGTGTGTGGA
GGGGAGAGCAGGGACGGGAAGGAGAGCAGCGAAGGGGAGGGAGGACAGCAGGGGAGGGGAGAGCCGGGAAGGAGAGGAGAGCAGGGGTG
GGGAGGTTCTGGAAAGGGTGTGCAGGGGAGGACGCGCCTCGGTTATGGGACTGGAGCCCCTTCCCAGGAGGACCCCCAACAATCCAGAG
GTGCCTGTTAGGATTCAGAACATGCTTTTTTTGTTTGTTTTTTTGAGACTCACTCCCTCACCCTGGCTGGACTGCAGTGGCGTTATCTC
GGCTCACTGCAACCTCTGCCTCCTGGGTTCAAGTGATTCTCCTGCCTCAGCCTCCCAAGTAGCTGGGATTACAGGTCTGCACCACCACG
CCTCGCTAATTTTTATATTTTTAAAATTTATTATTTATTTATTTTGAGACCCAGTCTGCAGTGCAGTCGCGTTATCTCGGCTCTCTGCA
ACCTCTGCCTCCTGGGTTCAAGCGATTCTCCTGCCTCAGCCTCCCCAGTAGCTGGGACTATGTGTGGCAGCCACCATGCCTGGCTAATT
TTTTTGTATTTTTCATAGAGACGGGTTTCACCATGTTGTCCAGGCTGGTCTTGAATTCGTGGCCTCAAGTGATCCGCCCACCTCAGCCT
CCCACAGTGCTGGGTTTATAGGTGTGAGCCACCACACCCGGCTAATTGTTTTGTATTTTTACTAGAGACGGAGCTTCACTATGTTGGCA
AGGCTGGCTCGAACTCCTGACCTCAAGTGATCCGCCCACCTCACCCTCCCAAAGTGCTGGGATTACAGGCGTGAGCCACTGCGGCCCAG
CAGAACACGTTCTAGGACCCTTGTTCATGTGTCCATCATGGACAGGAGGACGTGCGGGCCATAGGGACCCTGGCTCATTCCGGAGCCGG
GACTGGAGGGTGGGGCGTCACCCTTGGGAACACCCCTGCCCACCCTCCGCTGCCCACGGTAGGGGTGGGGAGCCAGGCTTTGGCCCCCA
CTTGATAAAGTCCCCTCCCCAGACTCCACAGGCAAATACTGGACGCTCGGCAGTCACTCCGCGGTCACCACCAGCGGCGACACTCCTGT
GGACTTCTTCTTCGAGTTCTGCGACTATAACAAGGTGGCCATCAAGGTGGGCGGGCGCTACCTGAAGGGCGACCACGCAGGCGTCCTGA
AGGCCTCGGCGGAAACCGTGGACCCCGCCTCGCTCTGGGACTACTAGGGCCGGCCCGTCCTTCCCCGCCCCTGCCCACATGGCGGCTCC
TGCCAACCCTCCCTCCTAACCCCTTCTCCGCCAGGTGGGCTCCAGGGCGGGAGGCAAGCCCCCTTGCCTTTCAAACTGGAAACCCCAGA
GAAAACGGTGCCCCCACCTGTCGCCCCTATGGACTCCCCACTCTCCCCTCCGCCCGGGTTCCCTACTCCCCTCGGGTCAGCGGCTGCGG
CCTGGCCCTGGGAGGGATTTCAGATGCCCCTGCCCTCTTGTCTGCCACGGGGCGAGTCTGGCACCTCTTTCTTCTGACCTCAGACGGCT
CTGAGCCTTATTTCTCTGGAAGCGGCTAAGGGACGGTTGGGGGCTGGGAGCCCTGGGCGTGTAGTGTAACTGGAATCTTTTGCCTCTCC
CAGCCACCTCCTCCCAGCCCCCCAGGAGAGCTGGGCACATGTCCCAAGCCTCTCAGTGGCCCTCCCTGGTGCACTCTCCCCGAAACCCC
TGCTTGGGAAGGGAAGCTGTCGGGTGGGCTAGGACTGACCCTTGTGGTGTTTTTTTGGGTGGTGGCTGGAAACAGCCCCTCTCCCACGT
GGCAGAGGCTCACCCTCCCTCCCTTCCCTCGAGCGGCAGGCCCTGACGGCCACAGGGTCTGCCCCCTGCACCTTCTGCCAAGGTGGTCG
TGGCGGGCGGGTAGGGGTGTGGGGGCCGTCTTCCTCCTGTCTCTTTCCTTTCACCCTAGCCTGACTGGAAGCAGAAAATGACCAAATCA
GTATTTTTTTTAATGAAATATTATTGCTGGAGGCGTCCCAGGCAAGCCTGGCTGTAGTAGCGAGTGATCTGGCGGGGGGCGTCTCAGCA
CCCTCCCCAGGGGGTGCATCTCAGCCCCCTCTTTCCGTCCTTCCCGTCCAGCCCCAGCCCTGCGCCTCGCCTGCCGACACCTGGGCCAG
AGCCCCTGCTGTGATTGGTGCTCCCTGGGCCTCCCGCCTCGATGAAGCCAGGCGTCCCCCCCTCCCCCAGCCCTGGGGTGAGCCCCCCC
GGCCCCCCTCCTGCCAGCCTCCCCCGTCCCCAACATCCATCTCACTCTGGGTGTCTTGGTCTTTTATTTTTTGTAAGTGTCATTTGTAT
AACTCTAAACGCCCATGATAGTAGCTTCAAACTGGAAATAGCGAAATAAAATAACTCAGTCTGCAGCCCCAGGCCGGCCTCTCTGTGTC
TTCGGGCTGACGTGGGTGGGCGGGCTGAGGTCGCTGGGAGGGCTGGCGGGACAGGTAGGCGCCCTGCCTCCCCAGCTCAGTGCTGGGAG
TGTGCAGTGGGACGGAGGCCGTGGCTCCAGTGGGTGCTCCGGACCTCGTGGGCCCAGCACACCTCCTTAAGCGGCCCATCGAGCGCTGG
GAGGGGGTGGACTGTGGCCCATGCCACCCCCAGAGCCATTAGGAGGAGTTCTGTGGTGAGAAGTGGCTGTGGCTCCTCGTAGGCTACGT
CCACCATGCGGGGGACCTCGGGGGTGTCTGGCGGTGGCACGCTGGATGTTGAGAAGGCGCAGCCCAGGGAACACTCAAACCAGGAGACC
CCACATTATCCTCTAGTTCACATGTGCCCTTCGACTAGGGGACTTGGTGGTAGGGCCAGCTCCGCCCCCATACTGCGAGGATCCGGGCC
TTCCACTTCCCCAGACACAGCAGGTTGGGGTGCCCCTGGGGCTGGGAGATGCTGCCCTGGGCCCCTCAGCCGGCGGCCGTGGTAGGATG
GACCGGGCCGAGCCTCCAGACCCTGGCCAGGTCCCTACATCGGAACCACCAGCCCTGGCGATATCTGGCATATCAGAGGCTCAGGACTG
TTTCCAGACTTTACCAAGGCCACAGTCAGCGCGACCTGGGTTCTGAGCCTCCCTTTGCATCCTAGGCTGCAGGGACAGGGGCTGGGCAG
AGGCTCGCGGACCTCGTCGAGCTTCATTCACTCCCAGGTGCCCCTAAGGATAGCAGCTGCGAGAAGACTGAGGGGAGGAGCAGTAAGGC
GCAGCCTGGAGCGGAGGGACCTGGCGTCCAGTCATTGTGCGCTGGGAAGGCGGTGATGGCCACGGAATTTGGGACACGCGGGGCGTCCC
GGGGGCACCCAGGGCACTGCAGGCCGGAGCCCCCTGTTCCCCCGCATCCTCCCCGCCGTCGGCAGCAGAGCAACCGCTGATTGGCTGCT
GATGACGCAACTGGGAGGGCTGCGCTGTGATAGGTGGTCCCTCGGGGGCGTGGGGCCCAAGTCTTGAGATTGGCAGGGGCAGGTGGCTG
CTGCAGAGGGAAGTCGGGGTCACCTCAGCGAGGTTCCGGCGGCCACCCCTCTCCCCTCCCCCATGAAAGCCAGGCTGGAAGCCAGAAAA
ATCCCAGTGACTGGAAGGGACAGATTAATCCTCCGGAACCAAACTCTTTGAGACCTGGGGAGGAGGGTGGGAGGCACTGGGAAGGGGGA
TTGGGGGGAGCCTGGCTCATTTCCACCCATGGAGCTGACCTAGAATGACCCTAAGGTCCCCGGACCCACCAGCTGATTCCGAATTCCAT
TCATTCTGCAAAATTTGGCGCCTACCACGTGGCCGACATTCCCGAGGCTGCAGCCAATGAGCAGCATAGTCCGATGCGGGAGGCTGGAA
GGGCCGTTCACGAGATCTCTCGGGAGGAATCGGTAGGAGCTGCCCAACTGAAAGGGACGGGGGAATAACCCGGGGCGAGGGACCTGCCC
ACCCCCTGCCCTCTGGGGGGAAGCCAGGCCAGCGGCCGGGGGCATCTCTGGGGTCCCAGGTGAGATGCGCGTCGAGGGAGCCAGGCCAC
GGTTCTCCAGACTCGCGTGGAGGCCACGAGCGTCTTCTGGAGGAGCGGCCACTGCGCGGACCGGCAGACTCTGGGGCGCTCGTGCTCCC
CATCTCCTGACCTTTTCCTACCTTCAGTTCCCTCCTGTCAGGACAGGTTCTGGGGCTCGCCGCGGCAGGGAGCCAGGGCAGAGGGTGCA
GTTTCCGCTCCTGCCTGAGTCACCTTCGCTTTCTCAGCGATTCTTCATTCACAAGGGCGTGGCCCATCACACGCACTCACACACTTGTG
AACTTGCACGCACACACACATATCGACCTGTGCAAAGAGCCCCATGCACACCCAGGTACACGTGCATGTAGGCGTGCACACAGACACGC
ACAGATGCAGGCAGACCAACTCCCAGGAATCTGTCTCCAGCCTACAGGGATTCAGCACTCAGGGTACCAGCCTCTGTCCCGTGCACCCC
CATTCCCGGCAGTTCTCCCAGGTGGGTCGGGGGCCACTAGGGACCCCATACCCCGAGGGTGGCTCCTCCATTAAGCCTGCCATGGGGTT
GGGAGTGAGAGTCCCACCTCCCACTTCAGAGTCCCCAGTGAGTCAAGGCAAGTGCAGCCAGGGCTCCAAGATCCTTGTTGGGATTCACT
GTGATCCCCAAAGATCACGACTTAAGGGAAAACATTCAATTAAACTTGTTAGAGCAAAGAAAGGAGAGACTGGGCTGGGAGCAGTGGCT
AACGCCTATATAGTTCAGCTACTCAGGAGGCCCAGGTGTGAGGATTGCTTGAGTCCAGGAGGCTGAGGCTGCAGTGAGCTATGATGGCA
CCACCCACTGCACTCCAGCCTGGGTGACAAAGCAAGACCCTGTCTCAAAAAAAAAAAAAAAAAAAAAAAAAAAGGAAAAAGAGGCCGGG
CACAGTGGCTCACACCTGTAATCTACTAAAAATAAGAAAATTAGCCAGACATGGTGGTGGGTGCCTGTAATCTCAGCTACTTGGGAGGC
TGGGGCAGCAGAATCGCTTGAACCCACGAGGCGGAGGTTGTGTGAGCCGACATTACACCACTGCACTCCAGCCTGGGCGACAGAGTGAG
ACTCTGTCTCCAAATAATAATAATAATAATAATAAAATAAAAAGAAAAAAGAAAGAAAGAAAGGAGAGACTGAGCTATATTGCCCAGTC
CAGGCATGTCGGCAGTCTGGGCGACAGTGGGGCATACTCGCTGTCTCAGGGTCCCAGCCCTGGGTGCCCCCACTGCTCTCTGCTGTGAC
CTCCTCAGGAGGCCTCATTGGGAGGGACCCTGAGTCCTGCGGGGGGCTGTGGAGCTGTCACCAGCCCCAGGAGGACTCAGGCAAGTCCC
TCTCGTCGTCGAGGCCTCTGAGCTTCCCAGTGTGTCTCCACCGGACACCCCCCCTCCAGACCTTCTGCCCCAGCTCAGCCTCTTCCAAT
TCATTATCAGGGGGTGGGCGAGGGGACAGGAAGCCCCAGACCCAGCTGCCCCCCATTTCCTCCAGGGCCACAGTGGCCTCTCCCAGACT
CACGAGGATGGGGGCTGATCTCACTCTTTTTTTTTTTTGAGACGGACTCTCACTCTGTCGCTTTTTTTTTTTTTTTTTTTTTTGAGACG
GAGTCTCACTCTGTCCCCCAGGCTGGAGCGCAGTGGCGTGATCTCGGCTCACTGCAATCTCCACCTCCCACGTTCAAGCCATTCTCCTG
CCTCAGCCTCCTGAGTAGCTGCGACTACAGCTACGTGCCACCACACCTAACTAATTTTTGTATTTTTTAGTAGAGATGGGGCTTCACCA
TATTACCCTCCCAAAGTGCTGGGATTACAGGCATCAGCCACCATGCCCGGCCTTGACTTCACTCTTTTAAAAAAGTCAAATACTTGGTG
CTTGAAATCCCAGCACTTTGGCAGGCTGAGGCAGGAGAACTGCTTGAGCTCAGGAGTTTGAGACCAGCCTGGGCAACATGGTGAGACTC
CTGTCTCTGTAAAAACTACGAAAATTAGTCAGGCATGGTGGTGCGTGTCTGTCGTCCCAGCTGCTCAGGAGGCTGAGGTGGGAGGATGG
TTTGATCCTGGAAGCTTGAGACTGCAGTGAGCTGTGATTCCACCACTGCAGTCCAGCCTGGGCCGCAGAGCAAGACTCTCTCTCAAAAA
TAAATAAAATAAAATAAAAAGTCTAAAACTTTTCTTTCAAACGACGGCTTTTGGGGCTCATAAGGTGCGTGAAGATGGAAAGGGGGCCC
GGCAGTGCCCATTTCGGCTCCCTTGGGACCCCGGCTTCCCTGAAGACCCCTGAGCAATGGTTGGCTGGGTGCCTGGTCTCCCTGAACGG
GCTGTGGGCAACAAGGTTGTCTGGATGGGGGAGGGGCTCTGAGACCCTTGGGGGCAGCAGAGGGAGAAGGGAACAGATGGGGTCTCCCA
GGCAGAGAGGCCTGGAGGGGTGGGGCGGGGCAGGGCATTTGGCGTGAAACCCAAGCCCCTTAACCGGGCCCTAGCTTAAACCGGGAGGA
GGGGCCTCTGTGATGTGCAAATAATTAACACAGAAGCCTTCACTGTGCTCCCCTCCTCATCCCAGGACCCAGGTCTGACCCTCAGGAGG
GGAGGAGGTGAGCAAGGGGGGAACCCACCTTCCAGGGAACCCCCAGATAGTCCCAGGCACCAGGCAAGTGCTCTAAAAGGCTTTACTAA
TATTATTTGAAGAAACTAAGCTAATAAATAAATACGGTATAAAATCAAAGGGTACCCACAGATATAAACAAAAATCCACAGCTAACCCG
GCCCCAGCCCTCTTTCCTTTGGAGGTAACTGTGTTAATCGTTTTTTACACCCCTCTGGAATTTTTTTTTTTTTTTTTTTTTTTTTTTTT
TTTTTGGGAAGGAGTCTGGCTCTGTCGCCCATGCAGCGCAGTAGCGTGATCTCGGCTCACTGCAACTTCCACATCCCGGGTTAAAACGA
TTCTCCTGCCTCAACCGCCTGAGTAGCTGGGACTACACGGGCGCACCACCATGCCCGGCTAATTTTTTTTTTTTTTTTTTTTTTTTTTG
AGCTGGAGTCTTGCTCTGTCGCCCAGGCGGGAGTGCAGTGGCGCAATCTCGGCTCACTTGCAAGCTCTGCCTCCCGGGTTCACGCCATT
CTCCTGCCTCAGCCTCCTGAGTGGGTGGGACTACAGGCTCCAGCCACCACGCCCGGCTAATTTTTTGTATTTATTTTTTAATAGAGATG
GGGTTTCACTGTGTTCGCCAGGATGGTCTCGATCTCCCTTTTTTTTTTTTTTTTTTTAGACGGAGTCTCGCTCTGTCGCCCAGGCTGGA
GTGCAGTGGCATGATCGTGACTCACTGCAAACTCCACCTCCCGGGTTCACGCCATTCTCCCGCCTCAGCCTCCCGACTAGCTGGGACTA
CAGGCGCCCACCACCACACCCGGCTATTTTTTTGTATTTTTAGTAGAGACAGCGGTTTCACCATGTTAGCCAGGATGGTCTCGATCTCC
TGACCTTGTCATCCTCCTGCCTCGGCCTCCCCAAGTGCTGGGATTACAGGCGTGAGCCACCGCGCCCGGCCTCTTGTTTTTTTTTTTTT
TTTTTTTTATAGACTTCTAAGGAAAGCAACACTAAGAAAATATATTTTTAAGAAATTATTCAGCCAGGCGTGGTGGCTCACGCCTGTAA
TCCCAACACTTTGGGAGGCTGAGGCGCCTCCATCACTTGAGGCCAGGAGTTTGAGACCAGCCTGGCTCACATGGTGAAACCCTGACTCT
ACTAAAAATACAAAAAATTAGCCGGGCACAGTGGCATGCTCCTGCAATGCCAGCTACTTGGGAGGCTGAGGCAGGAGAATCCCTTGAAC
TGGGGAGGCAGAGGTTTCAGTGAACCAACATCGACCCACTGCACTCCAGCGTGGGTGACAGACTGAAACTGTCTCTCAAAAAAAAATTT
TTCTTTTTGGAGATGGGGTCTTGCTCTGTCACCCAGGCTGGAGTACAATGGGGTGATCTAGGCTCACTGCAACCTCAAACTCCTGGCCT
CAGGTGATCTTCCTGCCTCACCCCCTGCAGTAACTGGCACTACAGGTGCATGTCAGAACACCTGGCTAGTTTTTAACTTTTTTGTCTGT
AGACAGGGTCTTGATATGTTGCCCAGGCTGGTCTCCATCACCTTCTGGGCTCAGTGATCCTCCTGCTTCAGCCTCCCAAAATGCTGGGA
TTACAGGTGTGAGCCACCATGCCCGACCCTGAGGGATACCTTAATGTTTATTTCTCACATTTCTTCTAGAACAAACATTGCCAGCGCAT
CCCAGACTCTGAACTGTGGCTCCCGACTGCAGCATGGAGGTCTTTCCAGGCACTCTGGGAAACATGCCACAAGCAGGGTATTAACCCAA
GACAGTCAGTCCCTCCAATGAGAAAAAGGAGCCGAGGGACTGTGCATGCCTCAGAGCCACATGCAGCAGAACTGGTGGGTTGAGGCGGC
ACTGAGCGCCTGGTGCCTCCTGCTGCCCCCCACCAGCTCCCACAGTCCTCGTCCTTGTCCTCCCCATCAGAAGGGAAGAGTGAATTGTC
TGTGGCATGTGCCAGTGTCCCCCGTTCTGAGGTTGGGCAGGCTGGGGAGCCCCACACATGGCTTGCTGCCCACTCTGCAGCCTCCTGGA
CTTAGTTCCAGGCTCTGCCCGATCCTCTGAGGGTGTGGCTTCAAAGCCCTGCTTGGGCCTAAGGCCATCCCAGGGCAGTGACTGACGGT
ACACTGAGATGTCCCTTGCGCACCAGTCCTAGGGTACCTGGGCTGGGGGTCTTCAGCTCAGGCCCCCTAGTGCCCGTCCTTTGGGCTGT
GCGCAGGTTTCCACAGCACTGGCCCTTTCTGACCTTACACCCCTGTGCATCCTCACTGCCCCTCTGTGAAGGTGAAAGTCACACTAAGG
ACTGTGCCTATGCTGGCCAGGGCACCCCACCGGCAGCACACCTCAGACGCCTCATCCTAGGCGCTCCTCACTTTTTTTTCTTTTTTTTC
CTTTTTTTTTTTTTTTTTTTTTCCCAGACACAGTCTTGCTCTGTCACCTACGCTCGAGTGCAGTGGCATGATCTCGCCTCACTGCGACC
TCCTCCTCCTCCTGGGTTTAAGCAATTCTCTTCCCTCAGCCTCCTGAGTAGCTGGCATTACAGCTGCCCGCCACCACGCCCGGCTATTT
TTGTAGTTTTAGTAGAGACCGGGTTTCACCATGTTGGCCAGGCTGGTCTCAAACTCCTGACCTTGTGCTCTGCCCCCCTTGGCCTCCCA
AACTGCTGGGATTACAGGCGTGAGCCACTCTGCGTGGCCCTGCTCATTTTCTTACCACAGCTTTCCAGAAATAAAGTTAGGATCGAAAG
AGAGAGAGAGAGATTGAGAGAGAGAGAGAGCACCACATTCAGATAGAGACAGGAGAGTTCTCAGCAAGAAGAGAACTCAGCTCAGAGTT
GAGAATGTCTCAACTCAGCTGTCTCCCTCCTGACACGTCCTGGGCTGAAGTGTCCCCTCACCCCTGCGGGGCTGTGACTCCACACTCCA
CACATGGGTCACAGCAGGGCTTGGCGTTTGGAGCAGGCCGGGTTGGTGGCTTGTCCTCATCTCTTGCCTGCAGCTGTGCTGGCCTGTGG
TTTGCTTTGGCTGCTACCATGTCCAGGGAGAGGTGCTTGTGTCCATGCCTGGCCTCTTGGAAGGCCACCACTGTGATCGACAGAGGAAG
GCCACCACTGTCATGGAGAGAAGCCGGGGCCAGACCGATGAGCGCTGAGAGCCCATGGGCAGCGCTTGTCGTCTCGGCCAAACCCCAGC
TGGCAGCTGATGGCAGCCACCCCGGGGTCCTCAGATATGGCACAGCTGCCTGGCTGAGCCTCGGCAAAGGGAAGCCTTGTGGAAAATAG
CAACTCAGGGATGTTGTGAGTAGTGGGCTGAGCTGTGGTTTATTTTTTAGAGACACGGTCTTGCTCTGTCACCCAGGCTAGAGTCCAGT
GGTGTAATCATAGCTCACTGCAGCCTTGACCCCCCAGGCTCAAGTGATCCTTGTGTCTTGGCCTCCTGAGTAGCTACGACTATAGGTGC
ATACCACCATGCCTCGCTTGTTTTTTGTTTTGATAGAGATGGGGGTCTTGCTAGGTTGCCCAGGCTGGTCTGTAACTCCTGGGCTCAAG
TGATCCTCCTGCTTTGGCCTCCCAAAGTGCTGGGATTACAGGCGTGAGCCACTGTGCTGGCCTTGGGGTGTGGTTTTATGTGGCCATAG
ATGACTGAGATGGGTGAGAAGCGGGACCTAGGGGAATATACAGGGCAGCGGGATCCGACAGGGGGACAGGAGGGCAGACAGCTCCTGGA
AGGCCTGGGAAAGAGATATCAGGGCTGGACCCTGGAGAAGTGAGGCTTTGAGCGAGGGGAGGAGATGGGGCCTGGCGGGGTACGGGCGC
TGCTCCTAGGTCTGAGGGCATCGGCGGTCTGAGAGCTTCCGGGAGGGAGGGCTGCCTCCCGGTGCTCGGTGCCCTGGCTCTCCTCCTTG
CTGCTGGCTGGCCTCCTCCTCCTCATCCATCTGTGTGTCCTGGAACACAGGGCACTGTCCCTCTGCCTGTCCAGAAAAGCACGTGCTCC
TTGGGGCCTGGCCTGCTCAGCCCTATGTCCCACCCCCTCCCTGCTCCTATTCCCAGGCCTTCGGGCCTCACTGCCTGGGGTGGCCGTGC
TCAGCCCCACCCTGTGGTGCCATGGCCTCCCCACCCCTGCCCCGAACTGCCCCTGGGACCAGTTCCTTCAGCTCCCAGGACAGGGCCTG
GCCATCCCTGTGGATTCTCCCTGTTCGTGGACCCCCATTGGTGCCCAGTCTCCTTGGACTGTCTTCTGAGCCTCTCTCAGATGTACCCC
CAACTGTACCCTTTCACCCAGAGGCCATTTGGCAGCGTCCTCCAGGACTCCTGCCGTGGGGGAAGTCTCCACCAAGGCCAACGCGTGAC
TGCATCCCGAGGAGCTGATTTAAGATCTGGTTCTCAGCAACACCTGGAACTGGCCAGTCAGCATCCCCGGCCCTGCGGTCTGCACTTGG
ACAAGGGTCAGGCAATGAGGATGAGCAGCAGGCGGGGCTTCTCCTGGAAGCCTTCCCAGCCTCCTGGGCCACTCCAGTGGGTCCCCTCC
ACAGACTCAGCCTGTTCCCTGCCACCTGGTGCCGGTTTCTCTGTGGCACCTACTAGGGGCCGACTGCCCCGGGACTGCCCAGCTGAGCA
GCGCCGTCCAGGTCTGCACCCAGCTACACTCAGGGCACGTGCACAGGTGAGCAGCCGGGCACGCAGGCACCTGCCAGGTGTGGGTGTGG
ACTACGTGCTCCCGG

| | |
|---|---|
| HUMAN SEQUENCE - mRNA | (SEQ ID NO: 5) |

GCGGAGGGTGCCTGCGGGCCGCGGCAGCCGAACAAACCAGCAGGGGCGCCGCCGCAGGGACCCGCCACCCACCTCCCGGGGCCGCGCAG
CGGCCTCTCGTCTACTGCCACCATGACCGCCAACGGCACAGCCGACCCGGTGCAGATCCAGTTCGGCCTCATCAACTGCGGCAACAAGT
ACCTGACGGCCGAGGCGTTCGGGTTCAAGGTGAACGCGTCCGCCAGCAGCCTGAAGAAGAAGCAGATCTGGACGCTGGAGCAGCCCCCT
GACGAGGCGGGCACCCCGGCCGTGTGCCTGCGCAGCCACCTGGGCCGCTACCTGGCGGCGGACAAGGACGGCAACGTGACCTGCGAGCG
CGAGGTGCCCGGTCCCGACTGCCGTTTCCTCATCGTGGCGCACGACGACGGTCCCTGGTCGCTGCAGTCCGAGGCGCACCCGCGCTACT
TCGGCGGCACCCAGGACCGCCTGTCCTGCTTCGCCCACACGGTGTCCCCCGCCGAGAAGTGGAGCGTGCACATCGCCATGCACCCTCAG
GTCAACATCTACAGTGTCACCCGTAAGCGCTACGCGCACCTGAGCGCGCGGCCGGCCGACGAGATCGCCGTGGACCGCGACGTGCCCTG
GGGCGTCGACTCGCTCATCACCCTCGCCTTCCAGGACCAGCGCTACAGCGTGCAGACCCCCGACCACCGCTTCCTGCGCCACGACGGGC
GCCTGGTGGCGCGCCCCGAGCCGGCCACTGGCTACACGCTGGAGTTCCGCTCCGGCAAGGTGGCCTTCCGCGACTGCGACGGCCGTTAC
CTCGCGCCGTCGGGGCCCAGCGGCACGCTCAAGGCGGGCAAGGCCACCAAGGTGGGCAAGGACGAGCTCTTTGCTCTGGAGCAGAGCTG
CGCCCAGGTCGTGCTGCAGGCGGCCAACGAGAGGAACGTGTCCACGCGCCAGGGTATGGACCTCTCTGCCAATCAGGACGAGGACACCG
ACCAGCAGACCTTCCAGCTGGACATCGACCGCGACACCAAAAAGTGTGCCTTCCGTACCCACACGGGCAAGTACTGGACGCTGACGGCC
ACCCGCGGCGTGCAGTCCACCGCCTCCAGCAAGAATGCCAGCTGCTACTTTGACATCGAGTGGCGTGACCGGCGCATCACACTGAGCGC
GTCCAATGGCAAGTTTGTGACCTCCAAGAAGAATGGGCAGCTGGCCGCCTCCGTGGAGACAGCAGGGGACTCAGAGCTCTTCCTCATGA
AGCTCATCAACCGCCCCATCATCGTGTTCCGCGGGGAGCATGGCTTCATCGGCTGCCGCAAGGTCACGGGCACCCTGGACGCCAACCGC
TCCAGCTATGACGTCTTCCAGCTGGAGTTCAACGATGGCGCCTACAACATCAAAGACTCCACAGGCAAATACTGGACGGTGGGCAGTGA
CTCCGCGGTCACCAGCAGCGGCGACACTCCTGTGGACTTCTTCTTCGAGTTCTGCGACTATAACAAGGTGGCCATCAAGGTGGGCGGGC
GCTACCTGAAGGGCGACCACGCAGGCGTCCTGAAGGCCTCGGCGGAAACCGTGGACCCCGCCTCGCTCTGGGAGTACTAGGGCCGGCCC
GTCCTTCCCCGCCCCTGCCCACATGGCGGCTCCTGCCAACCCTCCCTGCTAACCCCTTCTCCGCCAGGTGGGCTCCAGGGCGGGAGGCA
AGCCCCCTTGCCTTTCAAACTGGAAACCCCAGAGAAAACGGTCCCCCCACCTGTCGCCCCTATGGACTCCCCACTCTCCCCTCCGCCCG
GGTTCCCTACTCCCCTCGGGTCAGCGGCTGCGGCCTGGCCCTGGGAGGCATTTCAGATGCCCCTGCCCTCTTGTCTGCCACGGGCCGAG
TCTGGCACCTCTTTCTTCTGACCTCAGACGCCTCTGAGCCTTATTTCTCTGGAAGCGGCTAAGGGACGGTTGGGGGCTGGGAGCCCTGG
GCGTGTAGTGTAACTGCAATCTTTTGCCTCTCCCAGCCACCTCCTCCCAGCCCCCCAGGAGAGCTGCGCACATGTCCCAAGCCTGTCAG
TGGCCCTCCCTGGTGCACTGTCCCCGAAACCCCTGCTTGGGAAGGGAAGCTGTCGGGACGGCTAGGACTGACCCTTGTGGTCTTTTTTT
GGGTGGTCCCTGGAAACAGCCCCTCTCCCACCTGGGAGAGGCTCAGCCTGGCTCCCTTCCCTGGAGCGGCAGGCCGTGACGGCCACAGG
GTCTGCCCGCTGCACGTTCTGCCAAGGTCCTGGTGGCGGGCGGGTAGGCGTGTGGGGGCCGTCTTCCTCCTGTCTCTTTCCTTTCACCC
TAGCCTGACTGGAAGCAGAAAATGACCAAATCAGTATTTTTTTTAATGAAATATTATTGCTGGACGCGTCCCAGGCAAGCCTGGCTGTA
GTAGCGAGTGATCTGGCGGGGGGCGTCTCAGCACCCTCCCCAGCGGGTGCATCTCAGCCCCCTCTTTCCGTCCTTCCCGTCCAGCCCCA
GCCCTGGGCCTGGGCTGCCGACACCTGGGCCAGAGCCCCTGCTGTGATTGGTGCTCCCTGGGCCTCCCGGGTGGATGAAGCCAGGCGTC
GCCCCCTCCGGGAGCCCTGGGGTGAGCCCCCCGCCCCCCCCTGCTGCCAGCCTCCCCCGTCCCCAACATCCATCTCACTCTGGGTCTCT
TGGTCTTTTATTTTTTGTAAGTGTCATTTGTATAACTCTAAACGCCCATGATAGTAGCTTCAAACTGGAAATAGCGAAATAAAATAACT
CAGTCTGC

| | |
|---|---|
| HUMAN SEQUENCE - CODING | (SEQ ID NO: 6) |

ATGACCGCCAACGGCACAGCCGAGGCGGTGCAGATCCAGTTCGGCCTCATCAACTGCGGCAACAAGTACCTGACGGCCGAGGCGTTCGG
GTTCAAGGTCAACGCGTCCGCCAGCAGCCTGAAGAACAAGCAGATCTGGACGCTGGAGCAGCCCCCTGACGAGGCGGGCAGCGCGGCCG
TGTGCCTGCGCAGCCACCTGGCCCGCTACCTGGCGGCGGACAAGGACCGCAACGTGACCTGCGAGCGCGAGCTGCCCCGTCCCGACTGC
CGTTTCCTCATCGTGGCGCACGACGACGGTCGCTGCTCGCTGCAGTCCGAGGCGCACCGCCGCTACTTCGGCGGCACCGAGGACCGCCT
GTCCTGCTTCGCGCAGACGGTGTCCCCCGCCGAGAAGTGCAGCGTGCACATCGCCATGCACCCTCAGGTCAACATCTACAGTGTCACCC
CTAAGCGCTACGCGCACCTGAGCGCGCGGCCGGCCGACGAGATCGCCGTGGACCGCGACGTGCCCTGGGGCGTCGACTCGCTCATCACC
CTCGCCTTCCAGGACCAGCGCTACAGCGTGCAGACCGCCGACCACCGCTTCCTGCGCCACGACGGGCGCCTGGTGGCGCGCCCCGAGCC
GGCCACTGGCTACACGCTGGAGTTCCGCTCCGGCAAGGTGGCCTTCCGCGACTGCGAGGGCCGTTACCTGGCGCCGTCGGGGCCCAGCG
GCACGCTCAAGGCGGGCAAGGCCACCAAGGTGGGCAAGGACGAGCTCTTTGCTCTGGAGCAGAGCTGCGCCCAGGTCGTGCTGCAGGCG
GCCAACGAGAGGAACGTGTCCACGCGCCAGGGTATGGACCTGTCTGCCAATCAGGACGAGGAGACCGACCAGGAGACCTTCCAGCTGGA
GATCGACCGCGACACCAAAAAGTGTGCCTTCCGTACCCACACGGGCAAGTACTGGACGCTCACGGCCACCGGGGGCGTGCAGTCCACCG
CCTCCAGCAAGAATGCCAGCTGCTACTTTGACATCGAGTGGCGTGACCGGCGCATCACACTGACGGCGTCCAATGGCAAGTTTGTGACC
TCCAAGAAGAATGGGCAGCTGGCCGCCTCGGTGGAGACAGCAGGGGACTCAGAGCTCTTCCTCATGAAGCTCATCAACCGCCCCATCAT
CGTGTTCCGCGGGGAGCATGGCTTCATCGGCTGCCGCAAGGTCACGGGCACCCTGGACGCCAACCGCTCCAGCTATGACGTCTTCCAGC
TGGAGTTCAACGATGGCGCCTACAACATCAAAGACTCCACAGGCAAATACTGGACGGTGGGCAGTGACTCCGCGCTCACCAGCAGCGGC
GACACTCCTGTGGACTTCTTCTTCGAGTTCTGCGACTATAACAAGGTGGCCATCAAGGTGGGCGGGCGCTACCTGAAGGGCGACCACGC
AGGCGTCCTGAAGGCCTCGGCGGAAACCGTGGACCCCGCCTCGCTCTGGGAGTACTAG

TABLE 2
(mouse gene Fosb; human gene FOSB)
Mouse genomic sequence (SEQ ID NO: 7)
Mouse mRNA sequence (SEQ ID NO: 8)
Mouse coding sequence (SEQ ID NO: 9)
Human genomic sequence (SEQ ID NO: 10)
Human mRNA sequence (SEQ ID NO: 11)
Human coding sequence (SEQ ID NO: 12)

| | |
|---|---|
| MOUSE SEQUENCE - GENOMIC | (SEQ ID NO: 7) |

CTCACTCCGCCAGTCTGAAGCATCCGGGCTCTGTCTGTGAAACAGTCCGCGAACTGGGAGGGTCTACCCACTGGCCCCAGCACCCCTCC
TCCCAAGGCCCCCCATGCTGCTGCTTCATGTGACCAGGGCTGGGCCGAATCTCTGAGCAGACAGAGAGAAGCATGGTGTGACCGCATGA
GTGGAGTGAGTTCTGGGGACAGTGCCCAGTTGTGCGATAGACAGCAGATTCCGGAGGACCACTGTGCATGTGTGTGTGTTGTTGTATTT
GGTTTGTGTTTGTGAGTATGTCTGTGTGTGTGTGTGTGTGTGAGAGAGAGAGAGAGAGAGAGAGAGAGAGACAGACAGACAGACAGAGA
CAGAGACAGAGACAAAGAGAGTTGTGTGAGGACCACTGTGCATGTGTGTGTGTTGTGTGAATGTTTGTGAGTGTGTGTGTTGTTGTATT
TGGTTTGTGTTTGTGAGTGTATCTGTGTGTGTGAGAGAGAGACAGAGAGACAGAGAGAGAGAGAGAGAGAGAGAGACAGACAGACAGAC
AGACAGACAGAGAGACAGACAGAGAGAGACAGAGAGAGACAGAGAGAGAGAGAGAGTTGTGTGAGGGCCTCCCAGGTGATCTGGGAAGC
TTGTGAATGTGAAAGTGCCTGTGGGTGGCTTCACCTCTGGCAGAGGCATTCTGCCTCCTGTATATTAACTATGTGTGCTCAGACAATCC
ACTCAAAGATGCTCTGGGGCTGCAGCTGATAGAGCAGCTTACTTAGTGTGCACAAGGCCGTGGGTGCAAACACCCCAGAACTACAATCC
CAGAACTCTCTCTGCGCACTGAACTCGACTTCCTCATTTGTTTCTTGTTTCTTTTTTTTGACGGAGTCTAACATTGTCCGGATTGCTTC
AAACTTTATAGCGGAGGAAGATCGATGGCAATTCTTCTGCCTCCACCTTCTGAGTGCTAGGATTGCAGGCATGTACCCTCATGCCCAGT
TGAGACAGTATCAGTGATTGAACCCAGGGCCTCACGCCTCCTGGAGAAGCCCTGTACCATGGAGCTACAAGTCTAGTTCCTTTTCTTGT
CCTTAAAAAAAATAAAAAGCTGGGTCTTCTTATAGCCCAGCCTGGCTTTGTACTCCCCATCCTCCTGCCTCAGCCTCCCAAGTGCTGGG
ATGATAGGCTTATGGCACGGCCATGATAGCACCTACAATTTCCAGAGTTGCCTGTCCTTTGCAGTGTACTATGCCTTGTGAGGGACAGG
CTCTGAGCGTCTGCTTGTGATCACACACCTGAGCACCTGTTTGCTGGGAGTTCTGTCCCCATTTCTTTGCCAGGCTGGAAGACACTGAC
ACACCCACCATGGTGACAGTACCCATGGACTTAAATGGTGTGACTCCCTCTGCTCTAATGGGAGGGTCTCAGTGCCCCGTATCTGCTGT
TTCTCAAATGGGGTAGGTAATACAGAGATAGGAGGGCTGACACAAAACATATCAAACACCAAGATAGCAAAGGCTAGAATGAGACCCTG
TCTCAAGCTTTCCCATAGAGAAGGGGAGTGTATAATTGCACCCACTCACAAGACTGTTGGCCATAAACAAGTTTGTATGGAAAATGTTT
ACAAAGTGCTTGGCACATCCAGGGGTGGTGGCGCACACCTGGATTCTCATACTTGGAGAGAAGAGCCAGGAAGATTAGGGACGGTTCCA
GTCAGGCCTACATATTGAGACCTTATCTCAAAAACAAAACAACACAACACACACACACACACACACACACACACACACCTCAAAAACCT
CTGGAAGTTCACATTTTGGGAAAGGCATCTGAATGGTAAAGTGTAGGGATTAAGAGTAGAGACTAGAGTCCCTCCATGGGACCGAGATG
TAGAAAAGAAAGAAAAGAGAGCTTGCTAAAGCAGGCCAGCCTAAGGGCAGACTCCTGAAAGGACTTGCTCCAAATGATTGTTGAGGGTG
GAGGACTCTAAGAAACTGGAATTGTGGGAAAAACTCTCGGGGAATGACAAAAGCCGGTTTGCAGGTTGGTGAGATTAGAAAGTAATGGC
CACAATATAGGGCACAATATACAGGCTGGTCTTGAACTTGTAATCCTCCTGCCTCAACCTCCCAACTATTATAGGTATATGTCACCAGC
TGGATGCTTTTGCCTGTCTTGTTTCTTTCTGTGCCCCAGGGCTTCAGTGAATAGGGATGGCTGCCGATTAAATAACTGAATTGCTAGCA
GGGTGGGTGAGTCAGGGCTTTAATCCCAGCCTTCAGGAGGCAGAGGCACACAGATCTTTGTGAGTTCGAGGCTAACCTGGTTAACAGAG
AGAGTTCCAGGACAGTCAGAGCTACACAGAGAAACCTTGGCTCAAAAAACAACAAAATATCTGAATGGCTGATTCAATGACTGGGCAGA
TGCACAGATAGACAGATGGACATGTACGATCCACAGACATGTACGATCTTTGCGATGGTGGGGAAGGCTTGGTAGCTAACACAGGCTGG
CTCAAGACCTGCTTCTGGGAACCCTTTCTTTCCTTTGTATCCTCAGCTCTGGGTCATGAAGGGTGTGGAGTGGGCCGGGAAGGGAGTGT
CAGGCTCAGTGGCTGCTTGCCTTGGATTCTGGCCCAGAGTGTCTGGAGGAAGAAGAGGGTATCTCGGCATCTGAGTCACCTTAGAGAAA
CCCTAGCCACATACCTGACTTCTGACATCACACAACCAGGGCAACCCTGGTGGCCGAGACAAGGAGACTGAATCATGGACTGAAATCAG
TCACCTTCGGCATCCCCACCTGGCCCCTAGTGACCCCCTGAGACACCCCCGAGTCGCTGTCTGCCTGTGTGGCCATCCAAACCACATCA
AGGTCACTAAACCCTCTCCAGCCTCACTCAGGGACAGGGTGGCCACTGCCACAGACCACCCACCACAGCATCTGCAGGACAGCAAGAGA
AAGTTGAGGGAGAAAAGGCAGAGGGTAGGCCCCAGTCAGGGAGAGGGAAGTGCCTGTGGGAGAAAGGGGAAGCTGTGGCTTGCGGCCAG
TAGCCTGGTGACCTCTCGCTGGGATCTGGAATGGGGTCTTAGTGTTTGCGCACAGAATGGCGAAGTTGCAAGCCACCCTCAGAGGACAA
TGGAGTCACCAGACAATCGGCTCTTACACTTGCACACGCAGCCACAGGCCCACAAAAGCCAGCGTGCGAGCCCTACCCTCCAGCCACCC
CAGAGCTACAGCTTGCCACACACACACAACACTCTCCCCACAAGACTCGCTTCCTTTACCCTATTAAGTGACATCCGTAATTCTCATTG
TGAAGATTCCAACTCTCGGTATTTTGATGATCTCATTTGATATCAATAACTTACACATTTCCAGTGCCAGGCACTCTTGTAGACTATTT
AGCTGTGCTAGGGATTGAACCCAGGGCTTCTTCCATTCTGGGCAAACATCCTGCCACTGACCTTGCTCCCAGACCCACGTGCCCATGGT
TTGTCCCCAGACCCCTGTTTTATATGTAATTCTGAGACAACGTCTCACTGTAATGCCCAGAGACCCTTCACCTCACTCTATTACCCAGC
CTTGAACTCACTCATCCTTCTGCAGGGATTGTAGGCATGACTAGCTAGACACAGCTAAGTATCTGTTAGAGCGTGTATCTGTACCTTGG
GGCAGACATCTGTCCAACTGTATATGTCTATACATTGTTAGGTTTCAAACAAATGGCTGAGGTGCCTATTTAACATATCAAGAGGACCT
GGCCCCCAATTTCTCCCTGCAATAACTCAGCTCCTATATGCCCTCCTGGCTGGCATACCCCACCCGCTATCCTGAACTTCTCCAGCCCA
GGGGCTGGGCTGTTCATCCCTATGTAATCCAACTATTTTAGCTCTCCCCTCTCTTTCTACCTTAAGGCTGCACCTGGCTCCCATGGCTC
CCCTCCCTCTCTCCCCACACGGCTCAGGCTTAAGTCGACTCTCGACTATCCTAGAGGTCCCTGCCTCTGCCTATGCTCTCCCACATATC
TATAATAAACTCTCCTCTCCATACCTAGGAACAGTCATGTTCCCTTCCTTTCTCTTTCTTAATTTCCTTTTCTTTTCATTCATACATAC
GTTTCTTTGGATTCTGTATTCTATTCTCCCTCCTTAAAAATATACACAGCTAGCCAGGCATGGTGATGCACACCTTTAATCCCAGCACT
CGAGAGGCAGACACACGTGGATCTCTGTGAGTTCGAGACCAGCCTAGTCTACAGATTGACTTCCAGGCCTGTCTGGCCTATACAGTGAG
ATCCTCAAAAAGAAAAATGTACACACACTACCCCCTTCAAGTCAGAAGACCCTGAGACACAAGCCCATCCATCGCCGGGCAGCATGGGA
AAAGCACCTTGTTACAAATGCCTGGAGTCAGGTCTGACACTCACACACAAGGTCCCTGCCACCAGTTGATCCAGCACCTACATTCGAAC
GATCAGAGAGGTCCAGATAACTGTGCTGCCCTGTCAGACCTTATGAATGAAGACCTGAATGGAGGCTGATGTCTGTAATTCCAGCACTC
GGGACCCACTCAGGACGCAAGGAGGATTGGAGGGACTTAGCTGGAGGCCAGCCTATGCTATAGAGTAAGACTGCTTCAAACAAGAGAAA
TGAGAACTGAAAAGATTGCTCAGTGGGTAGAGGCGCTTGCCTGCAAGTCTAAGGAACTGAGTTCAATTCCTGACATTTGCATGATGGAA
GAAAAGAAACTCCTTCAAGGTCACACACACACACACACACACACTCAAAGAAGTAAAAATACTTTTTAATTTAAAAGAACAGAGAAACT
TGAACACATCGTCGGTCTGCCATGTGGCTCTGCACTAATGCCTGCCTTGGATGTATGTAGTCCTGGTTTTGACCCCACCATTATACAGA
CCAGGTGTGGTGATACAAACCTGTAATTCCAGCACTGGGGGCTGGAGCCAAGACGATGGGAGAAGTTCTAGGTGGCTCATCCTTGGCTA
AAAAAACATAGCCTGAGCTACCTGACACTGGTTGCTAGTGTGAGCTATCTGAGACTCTGCCTCACAAATAATTAAGTAAAAATATGGTA
GATTCATCACGACACCACCTACAGATTGCCTGTCCTGTGCTGGGACACAGGGCAGCTTCACACTAACCCTGATGGGAACACCTGGGAGA
CCACATGCTTGTTGCAAGGCGCTGGCTCATCTCAGACCAAAGCGACTTCAGGAGCCAAGTAGACCACAATCCGTGTGCTTCAGACAGTG
GGAAGGGCATACAGAAACCTCAGGAACTGAGGCACAGCAGGCAGATGGGAGGCTGGGACAAGGGACCACTGACAGACACCATACATCAT
ACCATCTTGTCCCTCTGAATCTCCATGGGCACCCCCTCTGGGAGGACTCCAGGACTGCAAGCAAAGGTTAACTGAGTCAGCCAATTCAA
CCCTGGCCTAGGGGGTGGAGGCAGGGGGTCTATCAGCGCGAGGAGACAGGACAAGGACACTCATGTATTGCCATAGGGATGGCTCTCTT
GTCAAACCATCAGAACACCCACAAACATCCCGGCTCACAGGGGTCTGCAGGCGTAGGGCTGGGATGTGGCCCGAGACTTGTCCCAACTG
AGGAATGTAGTGGCAAAAGTTTGGCCAAGGCAAGACCAGAGGACTCTGTATGCATGAAGGCCAGGGTGATGACATTTCAGGAAAGAAAA
GCACAGAATCAAGCATGGTAGGTAGTTGGAAAGTGGAAGCCCGNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNACCACATTTACTAAGCCCCTCCCTGCCAAAG
TAAGCAATAAATTATGGCTAAGAGTCTCCCCACTCCTAAGATGGAAAGATGCCAAGCTGCAAAGGAGATCCTGACAACAGACCAGCTAC
TTGGAAGACTCAGAACCTAGCCAAGCTGCCTGCAAGAGGTTTAGACCAACTAAGACATCTGGAGAGAGCACCCTCCAATCTGTGGAGCA
GTCTTCAGCCTCTGCAGGGTCCTCCAGGCTCCCAGCTTTGTAAGCTGTCACTCATGCTGGGGCGCGCTTGGGTGACGCAGCTGTCTTTG
AGTGACTTCTCCCGTAAGTCACCCCTCACCCATGCACACTCCTGTAAGTAACTCTAATAGAAAATTCATCGGTTCAGCAAATTGGACTT
TCGTCTCTTCTGAGCTGGCTCCCTGTCTAGGGTGAATGGACATGTGTGTGTGGCATCTCCCCTGGAAAAGTTTTCTTTCCCTAGCCTTG
GCCTCGGGTCTCCCAGGCTTTTTGCAGTTGCTGGTGGCATCCTTCTTAGTGGGTACTTAAGAATCACGACCCTGGCCAGCGATGCAGCA
GCCGTTGGCACAGCGCTCCCCTAGCATGCAGGAGGCCCCTGCATTACATCCCCAGTAATAAACCAGGCCTGGCGACAATATAGACGTGA
GAGCAAGATGTTCATTGTCATCCTTGGGTACATAGTAAATTCTAGTCTTTGTTTTAAAAACAAACAAACAAACAAACAAGCAGGACATA
AGATCAGATATGCCTGGCATGGTGGAAGGTGCCTTTAACTCCAACACTCAAGAGGTAGAGGTCATCCTGGCCTACAGAATGAATTCCAG
GCCAAAGGTGGTACATAATGAGACCCCATCTCAAAAACAGACAGAGAAAGGAGGAGGGCAAAGGGTAGAAAGAGGAGGGGATGTTCACA
ATAGGGGGAGGGGATGTTCACAATACGTGTATATATATTCTTTCCTTCTGCATGTGGATAGAACCCAGGACTTCACACATGCTAGACAT
TCATTCCACTTTAAGCTACATTCCCGCCCTCACATCCCCTTTTGTTTAAATCATTAATATTGATATTATTATTATTATTATTATTATTA
TTATTACTACATATGAGTACTGGTTGTTTTCTGTCTTTCCATGCATGTGGAGATCAGAGAACAATTTTTACGACTCATTTTTCTTCTTC
CACCGTGAGTCCCAGGGATCGCACTCAGGTTCTCAGGTATGTGTAGCAAGCTCCTTTACCTGTGAACCATCTTGCTGGGGCTGATACCT
GCTGATTGCTAAACACATGCCACCGATTATATTAATGCACTAGTATTTTATTTATTTATTTTTCGAGACAGGGTTTCTCTGTGTAGCCC
TGGCTGTCCTGGAACTCACTCTGTAGAACAAGCTCGTTTCGAACTCACAGAGATCTCCCTGCTTCTGCTGGGATTAAAGATCTGTGCTG
CTGCCGCAGCTGCCACCACCACCAGACACCACCAATTATTATTGTTCAATTATTATTGTATACACACTTTCTTTTCTGTCTCCTGAGCT
AAGTGGGAAGGAGAGGCTTCCAACACCATCAAGCCTGAAGAACTCACCGGGTGTGGGTTCCTACCTGCTCCAATTCTGCAGGTCCCCCA
GATTCAGGGACCCGGAAAAGCCACCTATCCTTTGCTTAGCTATGAGTACTGAAGTTTTAGATCAGCCAGGGAGCACATTTTCCAAGCTC
ACCGAGTCACACAGAATCCGTGACAAAGCTACTGGGGAGGGGACTGCATCCTTACCCATCCCTACACCTGGGGATATCACAGACTCTCC
AATTCCCCCTGAGCCTCAGTTTCCCCACCTACACTGATTATACCCCTCTCGCCAGAATCTGCACTGGGGACCTTCCTCTTTTTCACCAC
CAGGGGCGCTCCACCGCTTTGGGGAGGGTGGGGTCCCACGGGTATAAGCAGACCTCCCATCTGGAGTTCCACCTTCTCCAACCCGGGTC
AGCAGGGGCCTTCTGAGGGAGTTTAGGCGCCTGTCAATCTCAGCCTCCGGGACAGCGTGGAACTGCGCAGGCGCGGGCGGGTTCCGCAC
AGCGCAACAGCGGGCGCGCGCGGGAGCGAGGGATTCCCTCTGACGTAATTGCTAGGATACCAAACAAACACTGGGCCGCGCTGGCCGAG
CTCCTTATATGGCTAATTGCGTCACAGGAACTCCGGGGAGGGCGGGGCGGGATCCCCTCCCGCGAAGCCCCTCAGAACGCAGCCTTGGG
GACCCCCCAGACCCCCAGGGTCACACTATCGCCAGGTGGCAGGTGCACGAGGCCCTCGAGGGCGCCACAAGGAGCTCAGTCGGCGGGAA
GGGAGAGTTTGCGAGGTTTGTGCATGGAGTTGCGGGTGACGCAAGCGCGGGGGGCGGGTCCCGGAGGCATAAATTCAGCCCGGCAGCTC
CCGGTTTCATTCATAAGACTGGAGCGGCTACGCCGGGGACACCCCCACCCGGGGCTTGCTGGACTTGACTACTGGTCACTCTTTCTTTT
TCTTCTTCTTGGAGGCCGTGAAAAATTTATATATATATATTTATATATATTTTTAAACTGGAGAGAAAGTTTTGTGGGTTTCCTTTTTG
CAAGACTCTTTCTACATCGTTCTTCCGATTCGTCCCCACGATACACTTTCTTTCTGTGGGCTTCTGGATCGCGCCCCTCTGCTCTCCCT
CATTTCTTGGGACGCTTCGACAGGATTCCAACTCCATCTGAAGCGCACGCTGCACCGCGGCACTGCCCGGCGGGTTTCTCCGCGGGGAG
CGATCCCCGCGTCGCCCCCCGTGAAACCGACAGACCCTGGACTTTCAGGAGGTACAGCGCCGCTCTGAAGGGGATCTGGGATCTTGCAG
AGGGAACTTGCATCGAAACTTCCGCAGTTCTCCGAACCGGAGACTAAGCTTCCCCGAGCAGCGCACTTTGGAGACGTGTCCGGTCTACT
CCGGACTCGCATCTCATTCCACTCGGCCATAGCCTTGGCTTCCCGGCGACCTCAGCCTGGTCACAGGGGCCCCCCTGTGCCCAGGGAAA
TGTTTCAAGCTTTTCCCGCAGACTACGACTCCGGCTCCCGGTGTAGCTCATCACCCTCCGCCGAGTCTCAGTACCTGTCTTCGGTGGAC
TCCTTCGGCAGTCCACCCACCGCCGCCCCCTCCCAGGTAAGTTCTAGATCGTAAAGATTCTACTTTAGTGGTTGGGGCGGGGTGTTTCT
TTCAGGCTAAAGTGTAGATTGAGCATCCTCTCAAATAGAGACGCGCCAAAACCCAGGATCTGGGATTGCAATAGTTCGGTTTGCACTTT
GGTTTTTTGTTGTTTGCAAACTGCAAAGAATGGAGTGATGTTTGCAAAAGGTTATTTGCGCGGAGCGCGGGAAAGGAATGCAGCTGGGC
AAACGTTGGCGATGCCCGGTGCAAAGTATATACCCGGTGGTTAGCAGAAGCTGAGAACTTTTAGCCGAAAGCCGGCTCCCTAAGCCGAA
GCTAGGCAAGTAGGGGAAGAAAAAGAAACAAAAAATTCCAGAGAAGCTTCCAGGAGCCTCCTCCTCTTCCCTCTTCCTTCAAAACGCGG
ACTGCAAGTCCGCAGTCACCCTCCACCCAGCAAGAGTTAGGGCCTCGAACCCCGGTCACCCTGCCTCCGCCTCCTGCGCGGAGACCTAA
CGGGGGACCCGTGCGTAAAGGCTGACGCGCTGGAATCCTCCGTCTGACGCGGGGCACGCACAGCGCCCAGCGCCCCCTCCGCCCGCCCC
GCCCCTGACGTCCCGGGCACGTTCTATTTTGGAACGCCGAGGCCACGTTGCTAAGGGAGGGGGCAGCGTGGCTTTGTGATTGGCTGTCG
CGGCGCGAGCTTTAGCCAATCAGCGTTCCCTTCCTATTTGTACAGCGTAGCTCCCTTCCTTGCTTTTTGTGGTTCTTCCCGTGCTGGGG
GTCTCCAAGAGGGACAGCTAGGCGATTCTTGTCGCGATCGGGGGACTCGTTGTCACCCCATGGGTCTGCGACGACCTTGTGTGGACCTG
GTCCTGTTGTCATAAGCTAGAGGCTTTTGGCTGAGTGTTAGCGCCTCTAAGGGGGAACTGAAGGCCTCATCCCTTCTCCAGGCACACAT
ATACGTGCTCCCTGACCTCTAGACACTCAGTCCTTCCCAGGTGTTCAAACACTAGATGAGCTAGCCTACGGAGAGGCAGCCAGGTGGTC
TCTAAAAGGTCCGCCTTCCCTTACTTCCCAGGGCTCTGATTGGCCAGGGATTCACCCCTTCCCTCGCCACGCCCCCTAGACTAGTTAAG
CCTCTAGGATTCCACTTGCGGGAAGGGGGGGGGGCGCGTGATGGACGCTTCTTGGGTTCGGCGACCCAGATCCTATGTCACCCCATCCC
CTGCAAGACAGTCTGAGAGATTCTCGCTGTCACTTTTCTCTGCCTATCAGTTCACTGAAACCTGTCACTCTCACTCGGGAAGAGACAGA
CACTCGGAAGGGATGCTCTCAACTCTTAGGCCGGTCCCCCAACACCGTTGGAACTGGGATCTCCGCCCCTGCGGGAGCCCTCATGCAGT
GGGGGGTGTGTTTGTGTCTCAGTGGAGGAGAGGAAGGCTTGGGCTAAGGCCTCTCCCTCTCCCTACCTACTGTGGTGGGGGTGGGGTGT
TTTGGCTGTATGTGTGTGTGANNNNNNNNNNNNNNNNNNNNCTGACTTGAAGGGGGTAGTGTGTGTACATTTTTCTTTTTGAGACAGGA
TCTCACTGTATAGGCCAGACAGGCCTGGAACTCAATCTGTAGACTAGGCTGGTCTCGAACTCACAGAGATCCACCTGTGTCTGTGGCTC
CAGTCCCGGGATTAAAGGTTTGCATCACCAGGTTCTGTCCAGCCTCCGTCTCCCACCCACCCCCCCACACCTAAGAGTCACCAACCCGG
GGTGTGATTCACCACCCGCTGGAACCGTGCAACCTTTCCCCGAGGAAGAAGGAGGAGGTAGAAGGCAGTTGAACAGAATCTCTCATTAA
CCACTGCGTCACGGTGTAGTGGAAGGGTGGGTGTTGTGGCTTTTTGCCTGTGACACACACATCCACACCCGCTCACCCTGTGCTCACTC
ACAGGGGTCGGTGTGTGTTATGTGTGTTGGGCGTGTGTGTGTCCGTGGCTTTGTTTGTGTGTCTACGCCTGTGTGTGTATGTCTCACCC
CGTACGAGTGCGCCGGTCTCGGGGAAATGCCCGGCTCCTTCGTGCCAACGGTCACCGCAATCACAACCAGCCACGATCTTCAGTGGCTC
GTGCAACCCACCCTCATCTCTTCCATGGCCCAGTCCCAGGCGCAGCCACTGGCCTCCCAGCCTCCACCTGTTGACCCTTATGACATGCC
AGGAACCAGCTACTCAACCCCAGGCCTGAGTGCCTACAGCACTGGCGGGGCAAGCGGAAGTGGTGGGCCTTCAACCAGCACAACCACCA
GTGGACCTGTGTCTGCCCGTCCAGCCAGAGCCACGCCTAGAAGACCCCGAGAAGAGACAGTAAGTATGAGGCCTCAGGAGTTGGGATGG
AGGAGCCTAGCTAGGCATGTGGGCTCAGTTTGTACAGTGCTTGCTGCCATGCATGAAGATCCCTAGCACAGCATAAGCCAGGAGTGGTT
ATGCACACCTGTAACCCCAGCTCTCAGAACGTGGAGGCAGGAGGAGGAGGAGTTCGAGGCCAGCCTGTGCTACTTATGCAGTCCAGCCT
GCACTGCAAGACATCATTATTTTCAAAAGTTGGCCTTGGGGGGAGGTGGGTGAGGGAAGTAAGAGAAAGTGACAGTAATTTTATCACTT
AATAGTTGGAGGTTCCTCTGAGGCCTCAAGTCTGAAGGAACTTTACCATTCTGGCCAGTGAGGAGTAGGGGTTATTATTTGGGGTTCAG
GAGGAAGGAGTTTTCTTAGGGCTGATAGAGGTACCCCCAGATCTCATGGTCCTTATCTCTGACTCAGCTTACCCCAGAAGAAGAAGAAA
AGCGAAGGGTTCGCAGAGAGCGGAACAAGCTGGCTGCAGCTAAGTGCAGGAACCGTCGGAGGGAGCTGACAGATCGACTTCAGGCGGTA
AGGAGGAGTCTGGGGGTGTCTTGAGGCCGTGCTGGGAGCACTCTGCCTTGTTCTTCCCCCGTTTCTCACTGTGCCTGTGTCCTAAACGA
GGAAACCCCCTCTTAGGGAACAGGGGTCAGTATAGGCTGATGGAGTGGCTCCATATGCATGCTCAGACCCATGCCCACTTACTTTCGAC
TGTTCCCCACTTTCCCTGAATATGTCCCCACATGTCACCCTCCTGGCTTTCTCTCAGCCTAAGGAGACAAGCTGGAGGAGGTAATTCTC
TCACCTTCTTTCCTTCACTAAATAATAATCCATTTTGCCTTCCTGCCTCCATTTTTTTTTCCTGAGCTGGGGATCTACCTGTGTAGTTC
AGCCCTCCTCCCCCAACTTGATAGCCTCAAGTTTCACCCCTTGGCTGAGATGCCATCATCCTGACTGGCTCTGGCTGGAAACTATTTTG
TGCTAAGTCAATTCCTTACCTGCTACTCCAGCTATCTACAGTTCTGCCGAACTTGAGCTCGTGGCGCCCACCAAGCCCACTTCTTTCTC
TCTTCTACCTCAGTGCAACCCCCACACACACACACACACCTCATGCCTGCCCCTTGAAACCAGGGTGTGTCTCTGATTTCCCGTCGGGA
GGCTGAAGGAGATGGGTAACAGAACCTCATTAAAAACAACACATAAGCATTACCTACTGACTCAACAAACTGTAGTGTTTTTCTTTTTT
CCTCTCAAAAAATTATTTCGTTTGTTTATTTATTATTTGCTTATGTTTGAGTGAGTCCTGGTGCACCACAGCACACATACGACCTCAGA
GGGAAATTTTCATAGTTTGTTCTCTCCTTCCGTGTTGTGGGTGCTTGCTGGCAATCTCCTTCACTCAGTGAGCTACAATGCCCCCTTCT
GCCCTTTAAGGCAGAGTACTCCTTAGTACAGGGGGACCCTTTCCTCCGCCTCTCAAAGTTGAGATTACAAATGTTCACCATCACACCAG
GCTTGGAGTTCTTGCCTATCAGTGACGTCCACTCCTGCCTAGCTTCTTCCCAACCATCTCTTAGTCTGATGGGGAAACCGAGGCACGAG
TAGCATGGTCTACCAGGATTTCCTCTTAGGGGACGCTCCCCTCACTTGGGAGGGAGCTGTCCAGCCCCCTGGATCAGCAGCAAGAATGT
ATGAGTGTGGGGTTGGGCCCGTGAAGCTACTCTGTGTGGTCCCTGACCAGCAATTCTCCTTTCTCTGTCTCCTATGACCTGGCCCTGCT
GGGATCCATTAGGAAACTGATCAGCTTGAAGAGGAAAAGGCAGAGCTGGAGTCGGAGATCGCCGAGCTGCAAAAAGAGAAGGAACGCCT
GGAGTTTGTCCTGGTGGCCCACAAACCGGGCTGCAAGATCCCCTACGAAGAGGGCCCGGCGCCAGGCCCGCTGGCCGAGGTCAGAGATT
TGCCAGGGTCAACATCCGCTAAGGAAGACGGCTTCGGCTGGCTCCTCCCGCCCCCTCCACCACCGCCCCTGCCCTTCCAGAGCAGCCGA
GACGCACCCCCCAACCTGACGGCTTCTCTCTTTACACACAGTGAAGTTCAAGTCCTCCCCGACCCCTTCCCCGTTGTTACCCCTTCGTA
CACTTCCTCGTTTGTCCTCACCTGCCCGGAGGTCTCCCCGTTCGCCGGCGCCCAACGCACCAGCGGCAGCGAGCAGCCGTCCCACCCGC
TGAACTCGCCCTCCCTTCTTGCTCTGTAAACTCTTTAGACAAACAAAACAAACAAACCCGCAAGGAACAAGGAGGAGGAAGATGAGGAG
GAGAGGGGAGGAACCAGTCCGGGGGTGTGTGTGTGGACCCTTTGACTCTTCTGTCTGACCACCTGCCGCCTCTCCCATCGGACATGACG
GAACGACCTCCTTTGTGTTTTGTGCTCTGTCTCTGGTTTTCTGTGCCCCGGCGAGACCGGAGAGCTGGTGACTTTCGGCACAGGGGGTG
GGGCGGCCATGGACACCCCTCCTCCATATCTTTGTCCTGTTACTTCAACCCAACTTCTCGGGATACATGGCTGCCTCCGTGGGTAGGGT
GGGGTGCAACGCCCACCTTTGGCGTCTTGCGTGAGCCTGGAGGGGAAAGCGTGCTGAGTGTGGGGTGCAGCGTGGGTTGACCTCGAGCT
GCCATCCACCTCCAGAGAGACCCAACGAGGAAATGACAGCACCCTCCTCTCCTTCTTTTCCCCCACCCACCCATCCACCCTCAACCGTC
CAGGGTCACCAACATAGCTCTCTTTTCCTCCCTCGCGCCTTAGCTGATTAACTTAACATTTCCAAGAGGTTACAACCTCCTCCGGGACG
AATTGAGCCCCCGACTGAGGGAAGTCGATGCCCCCTTTGGGAGTCTGCTAACCCCACTTCCCGCTGATTCCAAAATGTGAACCCCTATC
TGATTCCTCAGTCTTGCCCTCCTGGGAAAACTGGCTCAGGTTGCATTTTTTTCCTCATCTGCTACAGAGCCCCCTCCCAACTCAGGCCC
GCTCCCACCCCTCTGCAGTATTATCCTACCTCCCTCTCACCCTCACCCCCACCCCAGGCGCCCTTGGCCGTCCTCGTTGGGCCTTACTG
GTTTTGCCCAGCAGGGGGCGCTGCGACGCCCATCTTGCTGGAGCGCTTTATACTCTCAATGAGTGGTCGGATTGCTGGGTGCGCCGGAT
GGCATTCACCCCCAGCCCTCCAAAACTTTCCCTGGGCCTCCCCTTCTTCCACTTCCTTCCTCCCTCCCCTTCACAGGGAGTTAGACTCG
AAAGGATGACCACGACGCATCCCGGTGGCCTTCTTCCTCAGGCCCCAGACTTTTTCTCTTTAAGTCCTTCGCCTTCCCCACCCTAGGAC
GCCAACTTCTCCCCACCCTGGGAGCCCCGCATCCTCTCACAGAGGTCCAGGCAATTTTCAGAGAAGTTTTCAGGGCTCAGGCTTTGGCT
CCCCTATCCTCGATATTTGAATCCCCAAATATTTTTGGACTAGCATACTTAAGAGGGGGCTCACTTCCCACTATCCCACTCCATCCAAT
TCCTTCAGTCCCAAAGACGAGTTCTGTCCCTTCCCTCCAGCTTTCACCTCGTGAGAATCCCACGAGTCACATTTCTATTTTTTAATATT
GGGGAGATGGGCCCTACCGCCCGTCCCCCGTGCTGCATGGAACATTCCATACCCTGTCCTGGGCCCTAGGTTCCAAACCTAATCCCAAA
CCCCACCCCCAGCTATTTATCCCTTTCCTGGTTCCCAAAAAGCACTTATATCTATTATGTATAAATAAATATATTATATATGAGTGTGC
GTGTGTGTGCGTGTGCGTGCGTGCGTGCGTGCGAGCTTCCTTGTTTTCAAGTGTGCTGTGGAGTTCAAAATCGCTTCTGGGGATTTGAG
TCACACTTTCTGGCTGTCCCTTTTTGTCACTTTTTTGTTGTTGTCTCGGCTCCTCTGGCTGTTGCAGACAGTCCCGCCCTCTCCCTTTA
TCCTTTCTCAAGTCTGTCTCGCTCAGACCACTTCCAACATGTCTCCACTCTCAATGACTCTGATCTCCGGTCTGTCTGTTAATTCTGGA
TTTGTCCGCGACATGCAATTTTACTTCTGTAAGTAAGTGTGACTGGGTGGTAGATTTTTTACAATCTATATCGTTGAGAATTCTGGGTG
GAAATGTCTGATCAGGAGAAGGGCCTGCCACTGCCGACCACAATTCATTGACTCCATAGCCCTCACCCAGGCTGTATTTGTCATTTTTT
TCATTTTGTTTTTTTGTATTTTGCACCTGACCCCGGGGGTGCTGCGGCAGTCTAGCACTGGGCAGCTCCCCTCCCCCCCTTGGTTCTGC
ACTGTCGCCAATAAAAAGCTTTTAAAAAACTGTATTCTTCAGGTCAAAGTGTCTGTTTTCCCTGCACATCTACTACATCGCTTCCTTTC
AGAAAAACGGAGTTTGGATTGCTAGGGAGGTCTTGCTCGCACTTAGTGGGACGCCTAATGAATCAGAACCTACAACGGGACTAAAAGGA
AGTGGAGACTTGCTAGGTTTTCCCATGTTCCCAGGCTGGGCCACCTACTTGAAAAAATAAGGGGCGGAAAAGTGTAAGGTACAAATTTG
GTGAAGGGTCTGGAAGACTTCATGATCGGAAAAGAATTTATTCACCTTGGGTGTGCAATGACTTCAGCAACAGCTAAGGGCAAGGTGTA
AAAGCTGGGCACACTTGTAAATCCTAGCATTTGAGAGGTGGAGGCAAGCGGATCACTGGTGGACTTCAGTCTCATGTGGATCGTAGATA
CCAAGCGCAAAGATCTGCTATGGGGACAGGGCTTGGTACACCAGGGGAGCCAGAAGTTCGTGGTGAGGGTAGTGGAGCCCAGCTCGAGA
CTCAGAGTTAGCCTCAGGGAGATTCTACAGGCAATGATGCAGAGTTCAGACGCTCCTTTGAAAGCACTAGAGAGCCGCAGCAGGTTTTG
AGCAGAGAAGGTTAGCGTTAGGTGGTCTCTTCTAGCCCATCCCAGGCTGAGGAGGACGCTGAGGGTTTCAAGAAGGATCGAGAATGGAA
AGCAGAGGAGAAAAAGGATCCAAGAGGCATGGAGGAGGCAGAACACATTTCTCTTCTTTAATAGCAAGCCTGGAAAGGATAACTTGCTG
CAGGAGGAGATGCTCACCAGTCGGGTGGTCTAGGGGGTTCTTGGAAAAGAGAACGCATTTGCTCAAGCCTCGGTTCCCCCATTCTCGCT
CTTCTGTCAGCTTGTCTTCCATTAAGTGTGTGTCTCAAGGCCACCCTGCTCAGGACTCCTTGTGAGACGACCTTCTATGCTCGAGTTCA
TTAAAAACACAATTGCCTGGTGCCCTGCTCTCTCCACTGGCTCAGTTACCTCAAAAGACCAGGGCTAAAGGTGTGATCACAACTCTATC
CCCATTACTGCTCCAACGCAGAGACAGGACTGAGCCGGAGTGAACAAATGAACAAAAATGACTAATAATGCATGCGTGATTAAATACAT
AAAAGAGCAGATGACTGGATGAGCAAATCGTTTAAGGAGAGACAGCAAGATCCTAGAATTTTGGAGACTAATTTAAATCCATCTTTGAG
ATGTATTTGGTCGGAAATTCCTGGGAGGAAAAAAAGTGTAAGTATGAAGAGAGAATAAATGAGAATAGGGGTGGCTTCAGAGAGGTTAA
CTGCGCGCTGGTCGCTTTTGTACAAGAATGTGAATTGCAGGGAGCAAAATGGGATAGATACTCCCGCCCCAAAGCTGGAATTGAACCAC
TCTGTCGCTAAACAGCTACAGGTTTGAAGCCTGCACCCCAGACCACTGAGGATCATCCGGGCGAAAGGAGCTATTTTCGGTTAGTTATA
TAAACCCCAGATACTACTACTTTTTTCACTTATGGTCATTATTTCTGGTATACAGTAGATAATTAATTTCAATGCTTTCGAACATTTTT
TTTCACTTTTTCTTGTGAACATGTGTTTCCTCAGTAAAGTGTTCCGTGAATGACTCTACTAACTAAAAAGTAAGTAGCTTCATTTGCAT
AGCGCCTTGCATTCTGGGAAGCAGCGCCTAAAGTGCCTGTCTCCCTAACTAAAAGCAGAATTTTTTGCAAAGTGAAAAGTCAGTTTTAT
TTTTGTTTGTTTGTTTGTTTGTTTGTTTTTAATGGAAAAACTTCTCACCCGCCCCATTCGTAGCAGAATTCGAGATTTTCTGCAAGCGA
GACCGTAGGGTCTGACGGCACGCGCCCGCAGAGCCACACCTGCCGTTGCTTTATAGAACTGCAAGTATGTAGGGAATCTACTCACTCCC
TAAGGTGATGGAGTTGACAACCAACTCCCCTTGCGTTTAGACGCTAAAAACCATCCCTTTTTATATCTATGTGATTAGCCCAGGGAAAC
TAAGGCTCAGACATGGATAATACCACAGCCGAGTTCCTGTAGCCCAACTCCCTAGGGGAAATGAAACCTACAGTTGTGGTTTTAATATG
CTTGGCCCAGGGGCAGTGGCCCTATTGGCAGGAGTGGCCTTATTAGAGGAGGTGTACCTTGTTAGAGGAAGTGTGTCACTTGGAGGCGA
GGTTTTGAGGTACGTATGCTCAAGTCTGGCCAGTGTGATCCTGGCTGTCTGCAGAACGCCGTCTCCTGGCTGCCTTCGGATCAAGGTGT
AGAACTCTCAGCTCCTTCTCCAGCACCATGTCTGCCTGCTTAATGCTTTGCTTCTTTCCATGACGATAATGAACTGTGCCTCTGAAACT
GTAAGTCAACCCCAATTACACAATTACATGTTTTCTTTTATAAGAGTTGCATATATATATATATATGTCACACTCTTTAGCTCTGGCTG
GCCTGAAATTCACTATGTAGCCCAGGATTGCCTGAACTTTGAAGCAATCTTCCTGCCTCAGCCTCCCAATGGTATTACAGGCATGAGTC
ACAACAAGCCATTTAAATCTTATGATGACTTATAAGAAGACAGAAAATCAGAGTTCCTTTACCTAGTTCACAGATCCCTACAATCTAAC
CTCGTTCGCTCCATAAACAGCCCTACCCCACCCTCCTGGAACTGCTTTGAGGAATGCTCCAGCCTCTCACAGGCACACTCCTCCTTGGT
TAATCTCTTCAGCCTGGTTGCCTTCCCTCCCCATGTCCATGTGGCCCAAAGCCTCTCATCCTGTTCTCAAATACCACTAGCTAGTAAGG
CCTCCCCGACCTGACCCCGGTTTAAATATTAGAAAAGGGTCACTTTCTCCCCTGCCACAGACAACCAAACCACCATATGCTTGTCACTT
ACTACCTGACTATGAAGGTGAATAGATGTCTTCACAACCTTTCTCTGAGCCTCAGTTTCCCCACCTCCATAATGCATCTGAGACACAGA
ATTCCCTAGAGCTGTGGTTCTCCTCATTCCTAGTGCTGGGACCCTTTAATACATTTCCTCATGTTGTGGTGACTCCACCACCACCATAA
AATTATTTTCATTGATACTTCATAACTGTAATTTTTTCTATTGTTATGAATAGTAATGTAAGCATTTGTGTTTCCCAGTGATCTTAGAT
GACCCTGTGGAAGAGTCATTCCACCCCCAAAGGGGTCCCCACCACAAGTTAAGAATTCCTGCCATAGAGCAATCACAAGGAACCAATGA
ATTACACTTTGGGTCGACTTTTGGGCTGGCCTCTGGGAGGCGNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNCTTG
GATTACAAACATGTGCCACCAAGTCTGGCTGCCCATTATGCATTTCTGTGGCAGGCGTTGATGGCTATTAAACCCAAGCCTTGTATATA
CTAGGCAACTACCCATCCACTGAACTACAGCCCTAGCATGGACCTCACAGGGCAATGGCCTGACATCTGGGAAACACTATCACAGAGTA
TAATAATAAAACCGTAGCATCCTTTTCACATTGAGCATTTCTGCTGGGTCACACTTCCAAAACCTTCTTATTGTTATCATGAGTATGTG
TGCATACATGTCGAGGTGAGAGGATGACGTCATAGAGACAGCTCTGTCTCCCATTCTTTATTGCCAATATTATTTTATGTTTTGTCTGC
GTGTATGTTTGTATACCAAAGTCCATGCAGTGCCTGTAGAGACCAAAAGGGGGCATCCCTGAGACTGGAGTTAGAAGTGGCTGTGAACC
ACTCACTCTGTGGGTGCTGGGAATCAAACCCCGGTTCTCTGGCAGAGCAACTGGTGCTCTTAACCACCGAATCAACTTCTCCAGCCCTG
CATTGCTTTAATCTTTTGGTCGATTCCCCCAATCCAACTCATACCGCCAAGTCTGAGTAGCAAGTGCCTTTATCTGGGAGACTCACTGA
TCTTATTTTTTTTTCCTTTCTTTTTCTTTCTTTTTTTCTTTTTTTTTTTGAGTCAGGTATGATTATTTAGATGGCCTCGAACTCACAGA
GATCCACCTCCCTCTGTTATGCCAGGCTTTTAAAAAACAACATTTATTTTTGAATGCATATATAGGGCTGTGCAAACACAGCCTGAAGG
AGTCGTTTCTGATTGCTCACAACGTGGTTCCAGGGATGGAACGCGGGTGACCAGCTTGAAGGTAGGTGCTTTCACCAGCCCCAAGAATT
TATTTTTTAACTTTATTTTTTTAATTAAATCATTAAATGACACATGTTCATTGTGGGTAGTGCAGGTATGCCATCGAGCACATGTAGAG
GTGACATTACTGCAGGCGGTCTCTCTACCATGGGGATTCAACTCAGACCACTGCTTCCTTTACCAGATCATCATTCTTGCATCCCCCAA
TCATTTCTTTTTGTTGATGGTTTTGTTTGTTTGTTTTAGTTTTGGGGGGTTTTTAAAGTTTTTTTCGAGACAGNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNATTAAAGGCGTGTACTACCACTGCCTGGGCA
TAACCGGCCTTTCTGCCTTATTTGTTTCGAAACGGAGTCTCATGTAGCCTGAGGTGGCCTCAAACATGCAATGTAGCTGCAGCTGGGAT
TACATATCTTCACCACCACAACCTGTTTTATGCACTGCTAAGAGATTAAACCTGGGCCTTCGTGCTAGGTAAGGACTCTATTAACAGAG
CTACAACCTCTGCCTGGTTTTTAGTTTTTCAGACAGGGCTTTGCGACATAGCCCTAACTGGCCTTGAACATGCAATGATCCTCCTGCCT
CGGCCTCCTGGGTCCTCAGATTGTTGATCCTACACCCACATATCTCAGACCTGAGGGTTGGCAGCTTGGGCCATGCTCTGATTTGGGAG
TGACATATTAACAGTAGTTTAATACGAATTTGAATCCACCTTACTTTTTTGGTGGCACTGACATTGAACTCATGGCCTTCCACAGGCTT
TTACGTGAGAAATGTTTTACCTCCCTTCCTTGAGCCATGAGCGAGCCCCTCACTGGTGGAGTCTATGCAGCTGGTCTGAAACTCATACC
ATCTTCCTGCCCCACTCTCCGAGTGCTGAGTGTCTACAGAGCTCCTGACTCTGCCTGGGACGTCATGTCTGTGGGACCTTGCCTATAGA
AGGGAGGCAGTCACACCACCCATTTTTGGCTTCCATGTCGGCAATGGACACCTTGCCATACTCACCTGATGTTGTCTGTACAGAAGGGG
GACGGTAAACAAAGCAACCACTCCTACAGATGCAGAGAGAGAGATGGGGGATGGGGGGTGTCAGGTTCTCCTGGCAAAGGGTCAGCTCC
GTTGAGCGCTTTTCCACTCCCGACCAAATTCCCAAAATAACGACTCTGAGACTCAATATTTCATGAACAAATGCTTGGGCTAGCTTGCG
CTTGTTCCCTGACAAGCCCCTATCTCATTTAACCCGTTTATTCTATTCTATGAATGCCACATGCCTGGTTACCTCGTCTCAGTTTCATG
AGACCCTCTCATCAGCACTAGGGCAAACCCTGATTCTATTACTCTCTTCCTGCAGGAGCTCGGGCCCCCTATTTCCTCCCTAAGCTAAT
AGCCCAACAGCTTTTCATTGACAGGTGATGCTTCCACACAGTGCACACGAAGATTCTCTTTACCCTCCATGTGNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNATTTTTGTAGAGAGGGCTGAGGTCAAGTTTAAGATTAGGAAAGAGAATCAAGAATTATCAAGGATAAAGGGA
GAGGTCAGGATTTACAGGGGAGCAGGGTTCATGGTCAGAATGAAAATGGATCAGAGAGGCCAATGCGTGAGCTGAGGTCAAGGGTCACA
ATGGACGGTCCGTCGGCGGGGGCTGGGCAGGGCCAAGCATTCTCACTGGAAACGGTTGGTGCCGTCGCCCCGGTGCACGCCCTGCAGCA
CTTTGCGGTAAACCCTGAGAGAGATGGTGGCGCAGAGACCCAACAGGGCCAGGTGCGCAGCCACGGACACGATGCTAAAGTGCAGGAGG
CAGAGGAGAGAGGCCATGAGGCCCGTGAAGACCGCTCCCGACGTCCTGGTGTCCTTCCAGTACAACAGGTCTGCCACTGTGGAAAAGAA
GACAATGTTGGGACCCGACCTGACCAAGCTCACTCCCTTTCTGTTTGTTTTTTAGAGATAAGGTCTCACTATGAAGCTAAGGCTGACTT
CAAACTATCAAACCCTCGCTGCTCTTGAACTCCTGATTATTCTAATTCATCCTTTCTTTTTCTTAGACTGAGTTAAGTTTGACCGGCTT
CAAACTCAAGACAAATACTCCTGCCTCTACCCTTTAGTGCTTCAAGGAGGTGCTAAGCATCAAATTTAGGGCTTCATGCATGCTAGGTA
AAACCCAACTGAGCTACTTCCCTGAC

| | |
|---|---|
| MOUSE SEQUENCE - mRNA | (SEQ ID NO: 8) |

ATAAATTCTTATTTTGACACTCACCAAAATAGTCACCTGGAAAACCCGCTTTTTGTGACAAAGTACAGAAGGCTTGGTCACATTTAAAT
CACTGAGAACTAGAGAGAAATACTATCGCAAACTGTAATAGACATTACATCCATAAAAGTTTCCCCAGTCCTTATTGTAATATTGCACA
GTGCAATTGCTACATGGCAAACTAGTGTAGCATAGAAGTCAAAGCAAAAACAAACCAAAGAAAGGAGCCACAAGAGTAAAACTGTTCAA
CAGTTAATAGTTCAAACTAAGCCATTGAATCTATCATTGGGATCGTTAAAATGAATCTTCCTACACCTTGCAGTCTATGATTTAACTTT
TACAGAACACAAGCCAAGTTTAAAATCAGCAGTAGAGATATTAAAATGAAAAGGTTTGCTAATAGAGTAACATTAAATACCCTGAAGGA
AAAAAAACCTAAATATCAAAATAACTGATTAAAATTCACTTGCAAATTAGCACACGAATATGCAACTTGGAAATCATGCAGTGTTTTAT
TTAAGAAAACATAAAACAAAACTATTAAAATAGTTTTAGAGGGGGTAAAATCCAGGTCCTCTGCCAGGATGCTAAAATTAGACTTCAGG
GGAATTTTGAAGTCTTCAATTTTGAAACCTATTAAAAAGCCCATGATTACAGTTAATTAAGAGCAGTGCACGCAACAGTGACACGCCTT
TAGAGAGCATTACTGTGTATGAACATGTTGGCTGCTACCAGCCACAGTCAATTTAACAAGGCTGCTCAGTCATGAACTTAATACAGAGA
GAGCACGCCTACCCACCAAGCACAGCTTGCTGGGCCACTTTCCTCCCTCTCGTGACACAATCAATCCGTCTACTTGGTGTATCTGAAGC
GCACCCTCCACCGCCGCACTGCCCCGCGGGTTTCTCGGCGCCCAGCGATCCCCCCGTCGCCCCCCGTGAAACCGACAGACCCTGGACTT
TCAGGAGGTACAGCGGCGGTCTGAAGGGGATCTGGGATCTTGCAGAGGGAACTTGCATCGAAACTTGGGCAGTTCTCCGAACCGGAGAC
TAACCTTCCCCCACCAGCGCACTTTGGAGACCTGTCCGGTCTACTCCGGACTCGCATCTCATTCCACTCGGCCATAGCCTTGGCTTCCC
GGCGACCTCAGCCTGGTCACAGGGCCCCCCCTGTGCCCAGCGAAATGTTTCAAGCTTTTCCCGGACACTACCACTCCGGCTCCCGGTGT
AGCTCATCACCCTCCGCCGAGTCTCAGTACCTCTCTTCGGTGGACTCCTTCGGCAGTCCACCCACCGCCGCCGCCTCCCAGGAGTGCGC
CGGTCTCGCCCAAATGCCCGGCTCCTTCGTGCCAACGGTCACCGCAATCACAACCAGCCAGGATCTTCAGTGGCTCGTGCAACCCACCC
TCATCTCTTCCATGGCCCAGTCCCAGGCGCAGCCACTGCCCTCCCACCCTCCAGCTGTTGACCCTTATGACATGCCAGGAACCACCTAC
TCAACCCCAGGCCTGAGTGCCTACAGCACTGGCGGGGCAAGCGGAAGTGGTGGGCCTTCAACCAGCACAACCACCAGTGGACCTGTGTC
TGCCCGTCCAGCCAGAGCCAGGCCTAGAAGACCCCGAGAAGAGACACTTACCCCAGAAGAAGAAGAAAAGCGAAGGGTTCGCAGAGAGC
GGAACAAGCTGGCTGCAGCTAAGTCCAGGAACCGTCGGAGGGAGCTGACAGATCGACTTCAGGCGGAAACTGATCAGCTTGAAGAGGAA
AAGGCAGAGCTGGAGTCGGAGATCGCCGAGCTGCAAAAAGAGAAGGAACGCCTGGAGTTTGTCCTGGTGGCCCACAAACCGGGCTGCAA
GATCCCCTACGAAGAGGGGCCGGGGCCAGGCCCGCTGGCCGAGGTGAGAGATTTGCCAGGGTCAACATCCGCTAAGGAAGACGGCTTCG
GCTCGCTGCTGCCGCCCCCTCCACCACCCCCCCTGCCCTTCCAGAGCAGCCGAGACGCACCCCCCAACCTCACGGCTTCTCTCTTTACA
CACAGTGAAGTTCAAGTCCTCGCCGACCCCTTCCCCGTTGTTAGCCCTTCGTACACTTCCTCGTTTGTCCTCACCTGCCCGGACCTCTC
CGCGTTCGCCGGCGCCCAACGCACCAGCGGCACCGAGCAGCCGTCCGACCCGCTGAACTCGCCCTCCCTTCTTGCTCTGTAAACTCTTT
AGACAAACAAAACAAACAAACCCGCAAGGAACAAGGAGGAGGAAGATGAGGAGGAGAGGGGAGGAAGCAGTCCGGGGGTGTGTGTGTGG
ACCCTTTGACTCTTCTGTCTGACCACCTGCCGCCTCTGCCATCGGACATGACCGAAGGACCTCCTTTGTGTTTTGTGCTCCGTCTCTGG
TTTTCTGTGCCCCGGCGAGACCGGAGAGCTGGTGACTTTGGGGACACGGGGTGGGGCGGGGATGGACACCCCTCCTGCATATCTTTGTC
CTGTTACTTCAACCCAACTTCTGGGGATAGATGGCTCGCTGGGTGGGTAGGGTGGGGTGCAACGCCCACCTTTGGCGTCTTGCGTGAGC
CTGGAGGGGAAAGGGTGCTGAGTGTGGGGTGCAGGGTGGGTTGAGGTCGAGCTGGCATGCACCTCCAGAGAGACCCAACGAGGAAATGA
CAGCACCGTCCTCTCCTTCTTTTCCCCCACCCACCCATCCACCCTCAAGGGTGCAGGGTGACCAAGATAGCTCTGTTTTGCTCCCTCGG
GCCTTAGCTGATTACTTAACATTTCCAAGAGGTTACAACCTCCTCCTGGACGAATTCAGCCCCCGACTGAGGGGAAGTCGATGCCCCCT
TTGCGAGTCTGCTAACCCCACTTCCCGCTGATTCCAAAATGTGAACCCCTATCTGACTGCTCAGTCTTTCCCTCCTGGGAAAACTGGCT
CAGGTTGGATTTTTTTCCTCGTCTGCTACAGAGCCCCCTCCCAACTCAGGCCCGCTCCCACCCCTGTGCAGTATTATGCTATGTCCCTC
TCACCCTCACCCCCACCCCAGGCGCCCTTGGCCGTCCTCGTTGGGCCTTACTGGTTTTGGGCAGCAGGGGGCGCTGCGACGCCCATCTT
GCTGGAGCGCTTTATACTGTGAATGAGTGGTCGGATTGCTGGGTGCGCCGGATGGGATTGACCCCCAGCCCTCCAAAACTTTCCCTGGG
CCTCCCCTTCTTCCACTTGCTTCCTCCCTCCCCTTGACAGGGAGTTAGACTCGAAAGGATGACCACGACGCATCCCGGTGGCCTTCTTG
CTCAGGCCCCAGACTTTTTCTCTTTAAGTCCTTCGCCTTCCCCAGCCTAGGACGCCAACTTCTCCCCACCCTGGGAGCCCCGCATCCTC
TCACAGAGGTCGAGGCAATTTTCAGAGAAGTTTTCAGGGCTGAGGCTTTGGCTCCCCTATCCTCGATATTTGAATCCCCAAATATTTTT
GGACTAGCATACTTAAGAGGGGGCTGAGTTCCCACTATCCCACTCCATCCAATTCCTTCAGTCCCAAAGACGAGTTCTGTCCCTTCCCT
CCAGCTTTCACCTCGTGAGAATCCCACGAGTCAGATTTCTATTTTTTAATATTGGGGAGATGGGCCCTACCGCCCGTCCCCCGTGCTGC
ATGGAACATTCCATACCCTGTCCTGGGCCCTAGGTTCCAAACCTAATCCCAAACCCCACCCCCAGCTATTTATCCCTTTCCTGGTTCCC
AAAAAGCACTTATATCTATTATGTATAAATAAATATATTATATATGAGTGTGCGTGTGTGTGCGTGTGCGTGCGTGCGTGCGTGCGTGC
GAGCTTCCTTGTTTTCAAGTGTGCTGTGGAGTTCAAAATCGCTTCTGGGGATTTGAGTCAGACTTTCTGGCTGTCCCTTTTTGTCACCT
TTTTGTTGTTGTCTCGGCTCCTCTCGCTGTTGGAGACAGTCCCGGCCTCTCCCTTTATCCTTTCTCAAGTCTGTCTCGCTCAGACCACT
TCCAACATGTCTCCACTCTCAATGACTCTGATCTCCGGTNTGTCTGTTAATTCTGGATTTGTCGGGGACATGCAATTTTACTTCTGTAA
GTAAGTGTGACTGGGTGGTAGATTTTTTACAATCTATATCGTTGAGAATTC

| | |
|---|---|
| MOUSE SEQUENCE - CODING | (SEQ ID NO: 9) |

ATGTTTCAAGCTTTTCCCGGAGACTACGACTCCGGCTCCCGGTGTAGCTCATCACCCTCCGCCGAGTCTCAGTACCTGTCTTCGGTGGA
CTCCTTCGGCAGTCCACCCACCGCCGCCGCCTCCCAGGAGTGCGCCGGTCTCGGGGAAATGCCCGGCTCCTTCGTGCCAACGGTCACCG
CAATCACAACCAGCCAGGATCTTCAGTGGCTCGTGCAACCCACCCTCATCTCTTCCATGGCCCAGTCCCAGGGGCAGCCACTGGCCTCC
CAGCCTCCAGCTGTTGACCCTTATGACATGCCAGGAACCAGCTACTCAACCCCAGGCCTGAGTGCCTACAGCACTGGCGGGGCAAGCGG
AAGTGGTGGGCCTTCAACCAGCACAACCACCAGTGGACCTGTGTCTGCCCGTCCAGCCAGAGCCAGGCCTAGAAGACCCCGAGAAGAGA
CACTTACCCCAGAAGAAGAAGAAAAGCGAAGGGTTCGCAGAGAGCGGAACAAGCTGGCTGCAGCTAAGTGCAGGAACCGTCGGAGGGAG
CTGACAGATCGACTTCAGGCGGAAACTGATCAGCTTGAAGAGGAAAAGGCAGAGCTGGAGTCGGAGATCGCCGAGCTGCAAAAAGAGAA
GGAACGCCTGGAGTTTGTCCTGGTGGCCCACAAACCGGGCTGCAAGATCCCCTACGAAGAGGGGCCGGGGCCAGGCCCGCTGGCCGAGG
TGAGAGATTTGCCAGGGTCAACATCCGCTAAGGAAGACGGCTTCGGCTGGCTGCTGCCGCCCCCTCCACCACCCCCCCTGCCCTTCCAG
AGCAGCCGAGACGCACCCCCCAACCTGACGGCTTCTCTCTTTACACACAGTGAAGTTCAAGTCCTCGGCGACCCCTTCCCCGTTGTTAG
CCCTTCGTACACTTCCTCGTTTGTCCTCACCTGCCCGGAGGTCTCCGCGTTCGCCGGCGCCCAACGCACCAGCGGCAGCGAGCAGCCGT
CCGACCCCCTGAACTCGCCCTCCCTTCTTGCTCTGTAA

| | |
|---|---|
| HUMAN SEQUENCE - GENOMIC | (SEQ ID NO: 10) |

CACTAAGCACTCTCACCACCCAATGCCTGGAGTGGTTGTAGTCAGTGAGTGACACATTGCACAGTGTGTGCCCCCTGGATTGGGGGTGG
GTGAGAGACAGCCCCCACAGTGAGTGGCACCCCTGGCCAGGGCCTGGGACAAGTCTGTATCCAAGGGTGGCTCTCTGCTTAGGTCTGTG
TTTGTACCTGGGTGTGTCTGTGTCTGCCCTACTCTGTGCATACTCATATATGTGAACCCGTGTGTGTGTGTGTAGTTGTGAGTGTGTGT
GGAAGAGGCTGCATGGCAGTGGAAGCTAGGTGTGTGATTCGTGTATCTGTGTGCCTAGAGTGCCTTGTTCTATAGATTTGGATGCCACT
CCAAGCAAGTCAGTGGGTCTTTTTGTTTTTTGTTTTTTGAGATGGAATCTCACTCTGTTGCCCAGGCTGGAGTGCAGTGGCACGATCTT
GCCTCACTGCAACCTCTACCTCCTGGGTTCAAGTGATTCTCCTGCCTCGGCCCCCCGAGTAGCTGGGATTACAGGTGCCCACCAATACA
CTCAGCTAATTTGTTGTATTTTTAGTAAAGATGGGGTTTCACCATGTTGGCCAGGCTGGTCTCAAACTCCTGACCTCATGATTCTCCTG
CCTCGGCCTCCCAAAGTGCTGGGATTACAGGCATGAGCCACCGCGCCCGGCCAAGTCAGTGGGTCTTTAGGAGCTGTTTCGTACGGTGT
GACTGTGAGTGAACCTTCGCACACGTGTCTGTACGGATATCTAAGAAATTCTTCAAGTAGGCCGGGCACAGTGGTTCAGGCCTGTAGCC
TTAGCACTTTGGGAGGCCCAGGTGAGTGGATCGCTTGAGCTCAGGAGTTCGAGAACAGCCTGGGCAACATAGTGAGACTTCGTATCTAT
AAAAAATATGAAAATTAGCCAGGCATGACAGTGGGCACCTGTAGTCCCAGCTACTACTTGGGGGGCTGAGGCAGGAGGATCCCTTGAGC
CTGGGAGGTGGAGGCTGCAGTGAGCTGAGATCGAGCCACTGCACTCCAGCCTGGGCGACAGAGGGAGACCCTGTCTCAAAAAAAAAGAA
AAGAAAAGAAAAAAACAAGACATTCTTCAAGTCCACAACTCTGAGGGTATCACTGTGAGAGCAGGGGTCCTAGCTCTACCCATTTGTGT
GTGTGTGGAGATGTGTAAGTCTATGTAGACAAAAGTGTGTGTCATTTACTGTGTTTGGGGTGTGAACACCTATGTGATGTGTTTGCACA
ACTGCACGTGTTTTGTTCTGTGTGTGTGATCGTGTGTTCAAATAAGTCATCTTGTCGCCGGGCGTGGTCGCTCATGCCTATAATCCCAG
CACTTTGGGAGGCCTAGGCAGGAGGACCATTGAGCCCAGGAGGTCCAGACTGCAGTGAGCCGAGATTGCGCCACTGCACTCCAGCCTGG
GCGACAGCAAGACCTTGTCTCAAAACAAAAGCAAAAACAAAAATAAACAAATAGTCATCAGGTGCCTGCACAGACAAAGGTGAAAGGTC
TCTCCCTGTTGAGATCTGTGGATAGGGTGTATATATGCACATCTCACCCTCTCTACGTGTCTATCTGTCTCTGTCCTCACGGCAAAAGA
GAGGTTGGCCGGGTGTGTGGTGGCTCACGCCTGTAATCCCAGCACTTTGGGAGGCTGAGGCGGGCGGATCACCTGAGGTGACGAGTTCA
AAACCACCCTGGCCAACATCGCGAAACTCCATCTCTTCTAAAAATACAAAAAAATTAACCCCCCGTGGTCGCACACGCTTGTAATCCCA
GCTACTCAGGGAGGCTCAGCCATTAGAATTGCTTGAACCCAGGAGGTGGAGGTTGCAGTCAGCCAAGATTGCGCCACTCGACTCCAGCC
TCGCCAACAGAACAAGACTCCGTCTCAAAAAAAAAAAAAAAAAAAAAAAGAGGCTCAGATATGTTTCTGTCTGAGAGTCTGTCAGAGTT
TAGGAATTACTAAATGAATGAATGGTGAAGATCCATTTACTCAACAAACATTTATTTATTTATTTATTTATTGACACAGAGTCTCCCTC
TGTCACCCAGGATGGAGTGCAGTGGCGCAATCTCCGCTCACTGAAGCCTCTGCCTCCTGGGTTTAAGAGAGTCTTGTGCCTCAGCCACC
AACTACCTGGGATTACAGGTGTGTGCCACTCCCCCCAGCTAATTTTTGTATATTTAGTAGACATCCGGTTTCACCATGTTGCCCAGGCT
GGTCTCGAACTCCTGCCCTGAAGTGATCTGCTCACCTCAGTCTCCCAATATCCTGGGATTACAGGCATGAGCCACCAGTCCTGCCTCAT
TTATTCTTATATTATATTATATTGTTTTTATTTTAAATTTTTTTGTAGTGACAGGGTCTTACTATGTTGACTGGGCTGGCCTCAAACTG
GCCTCAAGTGATTCTCCTGCCTCAGCCTCCCAAAGTGCTGGGATTACAGGCATGAGCCACTGTGCCTGGCTTGAACAAATATTTAATAA
CCACCTATTCGGTACCACCTGCTATGCTGGGGACAAGGCAGTGACCGGGATGGTTTTGGCCGAGCTTTCACCCAGCTCACAACCCAATG
AGGGAGACAAACCTATCCCCAGACAATGATGACCCCAGATTGGCAGAGTTGGAGGGAGGGACCCCAGGAGACGCGGCCTTGACTCAGCC
TGGAGATCAGGGAGGGCTTCCTGGAGGAGAGGTTATGGGAGTTAAGACTTGGAGGAAAAGACTATGAGCCACAGGAAGCGAAGGGAAGA
GTGACCCAGGCAGAGGGAAAAGCACCTGCAAAGGCCTGAAGTCCAGGGAGAGAGGCCAGGCATCTGGAACACAACGGGGAGAGGAGAGA
CGAGACTAGCGCACCCTCGGAGCAGGTCGTTCAGGGCCTCACGAGCCATCGGGAGCAGTATAATGCAGGATGCAGGGGTACTGGGAGGG
TCAAGCTAACAGTGTCCAATCTGGGTTACTCTGCTTCCCTGAAACTCCAGGGACATCTCCTCCAGGAAGCCCTAGGGATTGCTCAGTGT
GGAGCCGCTCAGCCACCCAGTCACACTCACTATTCAATGCTGAGCACTGAGGAGGCAGCCGTTACTAAATCAGCCTGGATTTGCCCTCC
CAGAGGTCACAGTCACAACAGTCAAAACACACACCTGCACGTGCACACTCATGAACGTCCCCCTCCCCTCCTCTGGAGCCTCAGAACCC
ACAGACCTTGTTCCTCCCAGGGGGTGTCCCTGGGGCCAAAGCTTCTGCACTACCAGGGGGCTTGTTGGAAATGCAAAAGATGGCTGGGC
ACAGTGGCTCACACCTGTAATCCCAGCAGTTTGGGAAGCTCAGGCAGGCACATCATTTGACGTCAGGAGTTCATTTGAGGTTGGGAGTT
CAAGACCAGCTTGGCCAACATAGCGAAACTCTGTCTCTACTAAAAATACAAAAAAATTAGCCGGGCGTGGTAGCACACGCCTGTAATCC
CAGCTACTTGGGAGGCTGAGGCAGGAGAATCTTTTGAACCCGAGAGGTGAAGGCTGCAGTGAGCCAAGATGGCACCACTGCACTCCAGC
CTGGGTGACAGAGTGAGTCTTAAAAAAATAATAATAAAATAGGCTGGGCGCGGTGGCTCACGCCTGTAATCCCAGCACTTTGGGAGGCT
GAGGCAGGCGGATCACCTGAGGTCAGGAGTTCGAGACCAGCCTGACCAAAATAGTGAAACCCCGTCTCTACTAAAAATACAAAAAAATT
TAGCTGGGCGTAGTGGCTAACGCCTGTAATCCCAGCTACAGGCTGAGGCAGGAAAATTGCTTGAACCCAGGGGTGCAGAGGTTGCAGTC
AGCCAAGATGGCACCATTGCACTCCAGCCTGGGCAACAACAGTGAAACTCCGTCTCAAAAAACAAAATACAAAAATTAGCCAGGCATGG
TGGCACACACCTCTAATCCCAGCTACTCGGGAGGCTGAGGCAGGAGAATCACTTGAACCCGGGAGGCAGAGGTTGCAGTGAGCTCAGAT
CGTGCCACTGCACTCAAGCCTGTGTAACAAGAGTGAAATTCCGTCTCAAAATAAAATAAAATAATAAAAATAAAAAGATGACCACAGCA
GATCTGCTGGCTCTCCAGGCAATTCGTGATCACCACGAATTGAGAAGCGCTGCTCTCAGGCTTAACCCCCCTGTACCTCAGTTTCCTTT
TCTGTACAAGGAGATCAAAACAGAATCCATGTAGAGCACTGACGTGGACTCAGGCTTAACTGAGATAATGAGTGTGAAAGTGCTAAGCA
CAGCTTCCTAAGTGCTTGTTTTCAGCACCTCCTCTTCCTCTTTTCTATTTTTTTATTTTTATTTTTTTTGGGGGGGGAACAGGGTCTCG
CTCTGTTCCCCAGGCTGGAGTATAGTGGCAGAGTCATACCTCACTGCAACCTCAATCTCTCAGGGCTCAAGTGATGCCCGTGCCTCAGC
CTCCTGAGTGGCTGGGACTACAGGTGTGTGCCACCATGCCCAGCTAATTTTTAAATTTCTTGTAGAGCTTGGGGGTGGGTCTCACTATG
TTGCCCAGGCTGGTCTTGTACTCCTGGCCTCTGGCGATCCTCTTACCTAGGCCTCCCAAAGTGCTGGGATTACAGGCGTGAGCCACCAC
ACCCAGCCCTCCTCCTCCTCTGTATTATTAAATTTCAGAGCTGGAAAGAAATTTCGAAGACCGCCATCACTCAACCTTCTTGCTCTACA
GATGGGGGAACTGAGGAACACAGGAGCAGAGGTTTGGCCAGAGGAAGGTGGGAAGGACGTGGTGTGCCTGGCCTTCACAGACTCTGCCG
GGGCCTCTTTACAGGATACTCTGAGGAAAGCCACGATGGGCACTGATACCCACCTCCACCCTGCGGGAGGCAGAAGGCTAAGGCTCCCC
CAGCCGTCTCCCCCAAGCAGAGCTGAGCCCTTGGGAGCCAGCCCAGAGATGCAACCCTCTCCCCCATGCTCAGGTCCTCCTCCTCCTGG
AAGCTCTCCTTCCAAGAGGAGCCTCTTATTTTATTTTTTATTATTATTATTTTTGAGACAAGGTCTCACTTTCTCACCCAGGCTGGAAG
TGCCGTGGCATGATCATGGCTCACTGAAGCCTTCCCCTCCCCAAGCTCAAGTGATCCTCTCACCTCAGCCTCCCAAATAGCTGAGACTA
CAGGCACACGCCACCACCCCTGGCTAGTTTTTGTATTTTTAGTAGAGATCGCCTCTTGCCATTGTCCAGGCTGGTCTTGAACTCCCGGG
CTCAAGTGATCTGCCCACCTTGGCCTCCCAAAGTGCTGGGATTACAGGCATGAGACACTGAGCCTGGCAAGGAATGAGCCTTATGGAGA
AGAACTTGATTTACCCACTCCATGCTCTGCAGGCAGGTGTGTCATGTCTGTGAGTGCAGGTGTGTGTGTGTGTGTGTATGTGTGTGTGT
GTGTGTCTCCAGGGCCAATTTCTCCCTTAGGCCCAGGGGCACAGTGCCTAGGGCCCATGATAAATTTAACCGACCCACGGATATGGGTC
AGGAGGGTATACATGTATAAAGTTCAGGAAGACTTTCTTTTAGTAACAAAACGAGCATGTACAATCTGTCAGGACCCTTATGATATCTT
CCCACCAGTCTCATGACTGGGCACTACATCTTGTTCTCCTTTGTCGGGTAGCCAAACTGAGGCCCCGGATGTCCTAGCTCAAGTTCACC
AAGCTACTTCGTGATAGAAGCGGAACATAACCCTAGGCCACCTAGCTTCTGGGTCTGCGATCGTCTGAGGATAAATGCCCAGTGTGTTC
GTGAACATGAGCATCCCTGTGTGAATAAATTGACATACATAAACCCATTTAATAAATTTCAAAGCCAGGCGCCGTGGCTTACTCCTGCA
ATCTCAGCGCTCTGGGAGGCTGAGCTGGGAGCATCGCTTGAAGCCAGCAGTTTGAGATCAGCCTGCAACAAAGTGAGACCCTGACTCTA
AAAACATTTTTTTGAATAAAAAAATTAGGCTGGACACAGTGGCTCACGCCTGTAATCCCAGCACTTTGGGAGGCCAAGGTGGGTGGATC
ACTTGAGGTCAGGAGTTTGAGACCAGCCTGGTCAACAGGGTGAAACCCTTTCTCTACTAAAAATAGAAAAATTAGCCAGGCGTGGTGGC
ACATGCCTGTAATCCCACCTACTGGGGAGGCTGAGGCCGGAAAATTGCTTGAATCTGGGAGGTAGAGCTTGCAGTGAGCCAAGATCATG
CCATTGCACTCCAGCCTGGGCGAAGAAACGAGACTCTGTCTCAAAAAAAAAAAAAAAAAATTAGTCAAGCATGGTGGTACCCACCTGTA
GTTGTTAGTTACTTGGGAGGCTGAACCAGGAGGATTACTTGAGGCCAGGAGTTCGAGGTTACAGTGAACTATGATTGCATCACTGCACT
CTAGCCTGGATGACAGAGCAAGATCCTATCTCAAAATAATACTAATAGTTCAAGGAACAGACTTTGAAGCCTGACACCCTGCATGGTGT
TATTCCAGCTTTGCTACTTACTTGCTGTGTGACTCTGGGTGAATAACTTAACCTCTCTGGGCTTCTGTTTCCCTTCCTGTAAAATGATC
ATTTGTACCTCACAGGAGTGTTGTGAGAATTAAATAAGTTAATATAAGCTCTTGGGAAGAATTAGCTCTTGTTATGGTTGTGTGAAGAG
CTTTACACCAGTCCCTCTGCAGGATGCATGGTTGTTGGTCTGTGTGTGTGTATATATATATATGTGTGTGTCTATGTGTGTATCTATGT
GTATATGTGTATATGTGTGTGTTGCTCTCTTGATCTCTGAGTGTTGTGTGTGTGTCAATGTGTGAGATCATATATGCACCTTTAGTAAA
GATCTAGGGGTCTCTGTGTGTTTTCGCGGTCTGTACCTGTCCTTGGCTGTACACCTGAACCCTACGTCTTCTTGTGTGTGTAGGGATGT
CTGTCATGTGTGTTGATAACCATATGACAGTGTCTGTTTCCACATGCCAGTGTGTGTCTGTGAAGCTCTTTCTATGTGGCTGTGGGTGA
TTGCCCACGCGTGACCTGGTGAGCGGCTGTGTGGAAGTCTGTGTTGCCTGTGTCGGCTGTGGAGTCTCTCGCCAGCCAGGCTAGCTCTT
TGCATGTTTCCTTTATTCCATGGAGGAGCAGATGAGGGGCTTGGATGAGACAGAAGAATGAGGGGTCACTCCCTGTCTGATGCTGGGGC
CGAGTCACTGCCTACTAGTGGCTGCTGTGTCATCTCCCAGTCTCTGTCCCTCCCTCCTGGCTGGTGGCACACACAGCGGGCGTGGAGGA
ACAGGGCAGGGGGTGCGCTTGAGGCTGGACTTTTGTCTGGAAACTTGAACCTCCACCTATGCCCCCCACGTCCCTCTAGTCTTCCACAT
CTCCCTACACCCTCCCTACTATTGGGGCAGGGAATCAGGACTCCCTGAAAATTAAGAACCATCGCCTTGGCTCTGCCACAGGCTTGCTG
TGTAGCCCGGAGACAGGCCCTGCCCCTCTCTCAACCTGTGAAATTCAATGTTTTAAAAGTGCTTCTTCCTTTGTGCCCAGGGCTGGGCC
AGGCTGTGCACTGGGAGGGAAGTCCTGGGTTCTCTGAGGTTTGTGAAATAGCTGCTAGCACCCTTCCCAGTGGGTCCTTAAGAGTTGGG
GGCTGTAGCATCTGAAATACCCAAGTCAGTGCAGGCATAGTGGCTCATGCCTGTAATCCCAGCACTTTGGGAGACCGAGGCAGGCGGAT
CACCTGAGGTTGGGAGTTCCAAACCAGCCTGGCCAACATGGTGAAACCCCCATCTCTATTAAAAATACAAAAGTTAGCCCGGGCACACT
GGCTTACCCCTGTAATCCCACCATTTAAGAAGGCCAACGCCCCACGATCACCTCAGTTTCGGAGTTTGACAGCAGCCTGACCAATATGG
AGAAACTCTGTCTCTACTAAAAATACAAAAAAACTACCCAGGCATGGTGGCACATCCCTGTAATCCCAGCTACTCGGGAGGCTGAGGCA
CGACAAGCACTTCAACCCGGGACGCGCAGGTTGCGGTGAGCCGAGATCGTGCCATTGCACTCCAGCCTGCCCAACAAGAGCAAAACTCT
GTCTCAAAAAACAAAACAGAACAAAACAAAAATTAGCCAGGTGTGGTGGCGCACACCTGTAATCCCAGCTACTCCGGACGCTGAGGCAG
CAGAATCACTTGAACCCAGGAGATGTAGGCTGCAGTGAGCCGAGATTGTGGCACTGCACTCCAGCCTGGGCAACAGAGTGAGACTCTGT
CAAAAAAAAAAAAAAAAAACGTCAGATTCATGAACCCCATTTGCTAAGAAGATTTGCTCAGGTAGTCACCTGTACCCCTGTGAGCCAAG
ATTGCAACAGTACATGCTAAGAGCACTAACAAGCACTTTGTAAAGCTTAGAAAGACCTGACAGGCCAGGCGCTGTGGCTCACGCCTGTA
ATCTCGACACTTCGGGAGGCCGAGGCAGGCGGATCACCAGGTCAGGAGTTCAAGACCATCCTCACCAATATGGTGAAACCCCGTCTGTA
CTAAACATATAAAAATTAGCTTCACGTGGTGGCCCGCGCCTGTAGTCCAGCAACTCGAGAGGCTAAGGTGAGGCGGAGGTTGCAGTGAG
CCCAGATCGCCACTGCACTCCAGCCTGGCGGCAGAGCAAGACTCCGTCTCAAAAAAAAAAAAAAAAAAAAAAAAAAAAGACCTGACAAG
TGAAAGCTACTAATATTGCCATTATCATTTAAAAAAACCCCAGACACAGGTTTTTCAGGGAGTTTCATCCAACCAGGCAGGTCTCAGAA
ATCAGAAAAGAAATGGAAACCGTAGCAAATCTGGAGTTTGACAATTCTGAGTTTGAATTTCTTGTGATGGGATCTTGGGCAAGTCATTT
AACCTCCCTGAGTATCATTTTTTTCTTTTATAAAATGAAGATTTTTCTCTCTTAACCTTCCACAGCTGTTTTAAGGATTACAAATCTTC
TACTGAAAGGCGTAGCACACGAGCTGTGCTTGGCAAGTGCCAAATACAAGGCATTAATTATTATTATTAGAATTAATAATAATATCCCC
TCCCTCTTACACATTCTTTGTCTCCGGGTGGATTAAAAGGTGGAACCAGACCCTACCAACACCATCAGAAGAGAGGCTTCTCTCTAAGT
TTCATTTCCCATCTCCTCCAAATCCGCATCCCTCCCAAACGCCGGACCTGTAAGGCCAGCAGGGTCCAAGACACACATCCTTTGCCCAG
CGGGGAAGATTAAAGCTAAAGCTCAGACAGCGAAAACATTTCCTAAGCTCGCACAGCCAATCAGGACAGAAACCAGGACGAGCCTCGGA
ATCCCTCCATTACCTCCACTTTCACCTGAGCATCACAGCCCCCTCGGGACTCAGTTTCCCCACCTACGTCACCACACCACACTAATCAG
GGTCTCCTTTTGGAGATCTGCTCTTCTTCTCGAATGGGGGCGCTGCACCATCCGTAGAACAGGGTAGGTGGGGGCGCCAGAGGTGAAGG
GGACCTCCAGCCTGGGGTCTTCCCCGCCCGCGTCAGCGGGGTCCCTGCGCGGCTAGTCTAAGCGCCTATTATTACCAGCCCCCGGGGCG
GCGTTGCACTGCGCAGGCCCGCGCGGGGCGCGGGCGCGCGCCCGAGCGAGCGAGGGATTCCCTCTGACGTCATTGCTAGGATACCAAAC
AAACACTCCGCCGCGCCGGCCGAGCTCCTTATATGCCTAATTGCGTCACAGGAACTCCGGGAAGGCCGGGCCGGGATCCCCTCCCGCCG
AGTGCCCCGGAACGCAACCCCCGAGACCCCCAGGGCCCCGAGGGTCATGCAAGTGACCAGATCGAGTCTAGAACAGACCTCTTGCTGGA
CAGTGCGGGACTCGATTTGGCGGGGCCCGACATTTGGGGAAGTTTGTCCAGCAAGGGGCGGGTGACGTAAGCAGGGGGGCGGGTCCCGG
GCATATAAATACAGCCTGGCGGGTCTGTGCTTCATTCATAAGACTCAGAGCTACGCCCACGGCAGGGACACGCGGAACCAAGACTTGGA
AACTTGATTGTTGTGGTTCTTCTTGGGGGTTATGAAATTTCATTAATCTTTTTTTTTCCGGGGAGAAAGTTTTTGGAAAGATTCTTCCA
GATATTTCTTCATTTTCTTTTGGAGGACCGACTTACTTTTTTTGGTCTTCTTTATTACTCCCCTCCCCCCGTGGGACCCGCCGGACGCG
TGGAGGAGACCCTAGCTGAAGCTGATTCTGTACAGCGGGACAGCGCTTTCTGCCCCTGGGGGAGCAACCCCTCCCTCCCCCCTGGGTCC
TACGGAGCCTGCACTTTCAAGAGGTACAGCGGCATCCTGTGGGGGCCTCGGCACCGCAGGAAGACTGCACAGAAACTTTGCCATTGTTG
GAACCGGACGTTGCTCCTTCCCCGAGCTTCCCCGGACAGCGTACTTTGAGGACTCGCTCAGCTCACCGGGGACTCCCACGGCTCACCCC
GGACTTGCACCTTACTTCCCCAACCCGGCCATAGCCTTGGCTTCCCGGCGACCTCAGCGTGGTCACAGGGGCCCCCCTGTCCCCAGGGA
AATGTTTCAGGCTTTCCCCGGAGACTACGACTCCGGCTCCCGCTGCAGCTCCTCACCCTCTGCCGAGTCTCAATATCTGTCTTCGGTGG
ACTCCTTCGGCAGTCCACCCACCGCCGCCGCCTCCCAGGTAAGTTTTTGATAGTAGGCGTGCTGCTTTGTAGGTTTTATTTTTTAAGTC
AAGGGTGAAAAGAATAAACCCCAACCCCCACAAAAAGGCGCATCAGAACCCTAGATCTGAGATGGAAAAGGCTCACAGCGCACTTTGCA
AACTGCAAAGAGTCGGGAGATGTTTGCAATTGGTTGCGTCCGTGGAGCGCAAGGAGGGAACGCGGCAGGGAGGGTAGGCTTTGGGGCGA
GGTGGGGGTCGGCTCCGTAATGCGCTGCTCAATGCAACGTGTATGCGGTAGCGGGGCTGAGAACTTTGAGCCGGCCCCGGGACTGCCCC
CTGCTCGGGTCCCAGACCTGAAGCTAGCGCAGTTAGGCAGGTGGGGGAAATCCCGGGGAAGCTTCCAGCAGTCTCTTTTCCTTTCCTCC
TCCTTCGGAGCGCCCACTTCGGTGCCGGGTCGCCCTCCACCCATCGGGAAGAGGGGCCTCGAACCCTCAGCCGCGCTGCCTCCGCCTCC
TGCGCGGAGACGTAACGGGGCACCCGTGCGTAACGGCTGACGCGCTGGAATCCTCCGTCTGACGCGGGGCACGCACGCCGCGCCGCGCC
CCCTTCGTCCGCCCCGCCCCTGACGTCCCGCGAGCGTTCTATTTTGGAACGCCGGGGCCACGTTGCTAAGGGAGGGGGCAGCCCGGCGT
TTCGATTGCCCGCCGGGGCGCACGCCTTGGCCAATCAGCTTTCCCTTCCTATTTCTAGGGTGCATTTTCCTTCCCCCCTCTCTGTCCCC
GGAACCCGTGGTTCCTTGGCGCCTGGGTCTCTTTTCGGCCCCTCTAGAGGCAGAGGGAGGGGATCCCTGTCGTGACAAGAGCGCCTGTC
TGCGACCCAATGGATCTGCGAGGCCCTTGCGGGCATCTAGTCCCTGGGCTCTCAGGAGAAGGGGGTGTCTGCTTGTGTGCTGGCGTTTC
TTGGAGAGATACGGTCGCTGTCACCCTCTTCTCCAGGCACACACAGACACATTCCCACTCCCTCTGCTCCTCATCCCCGGTTCCTCCGG
TGTGTCCCAAGACAGGACTAGAACCCCCAACCGAAGGGCAACCAGCCAGGTGGTCTCCAGGAGCTCCGCCCCCTCTGGGTTCCCAGGAC
TCTGATTGCTCGGCGACCCAGCCCTTCCCTCACCACGCCCCCCGAGAGAGTAGTTAAGCCTTCACACCAGTTCCAGGAGTCCATTTACG
GGAGGGGGGAGATGAGCGCTGCTGAGGCTTGGGGGCTCAGGTCCCGCACCATTCCCCCTCCGCGACAATCTGAGAGAGCTCCAGTGGTT
ACTTTTATCTACCTGTCCGTTCACCCTAAACTGTCACTCGTCAGTCTCACTCTGAGAAGAGACAGTAACTTGAAACGTTGTTCTAAACT
CCTAGGCCCGTCCCCCAAACACCCTTTTGACTGGGACCCCCGCCCCTGCATGGGACCTCGCGCAGAGGGGGGTGTCTATGTGTGTGAGT
GTAGAGGAAGGCTTGGCCTAAGGCCTCTCCTTCTCCCTCCCCTTGCCTCTGGGGTGGGGGTGGGGTGTTGTGGCTGTGTGTGTGGCTGT
GGCTCCGTCCCGGGGGTTCTGTCACCCGGCTGTGTCCAGCCTCCTCTCCACCCCCCATACCTAACACTCACCAACCCGGGGTGTGATTC
ACCACCCGCTGGAACCGTGCAACCTTTCCCCGAGGAAGAAGGAGGAGGTAGAAGCCAGTTGAGCAGAAATCCTCTCATTAACCACTGCG
TCACCGTGTAGTGGAAGGGTGGGTGTTGTGGCTTTTTCCCTGTGACACACACATCCACACTCCCTCACCCTGTGCTCACTCACGGGGTC
GGTGTGTCTTATGTGTGTTGGGTCTGTGTGTGTCGGTGTCTTTGTTTGTGTGTCTACGCCTGTGTCTGTATCTGTCACCCCGTAGGAGT
GCGCCGGTCTCGGGGAAATGCCCGGTTCCTTCGTGCCCACGGTCACCGCGATCACAACCAGCCAGGACCTCCAGTGGCTTGTCCAACCC
ACCCTCATCTCTTCCATGGCCCAGTCCCAGGGGCAGCCACTGGCCTCCCAGCCCCCGGTCGTCGACCCCTACGACATGCCGGGAACCAG
CTACTCCACACCAGGCATGAGTGGCTACAGCAGTGGCGGAGCGAGTGGCAGTGGTGGGCCTTCCACCAGCGGAACTACCAGTGGGCCTG
GGCCTGCCCGCCCAGCCCGAGCCCGGCCTAGGAGACCCCGAGAGGAGACGGTGAGTAAGGGACATCAGAACTTGGCCTGGGTAGGGGGA
AGCAAGAGAGGCAGGAAGTTTCTTATGAATGGAGGGGGGCTCCACTAAGGCCTCAGTGTTACAGAAACCCCAAGATCCTTGCTACACGA
GCGAGGACCCCAGGCTTGTTTTTTGGCTCTTGGGGGTTCTGAAAGAAGTAGAGGTTCGGGATCCCTCCACGAGTGCTGTATCCCCAAAT
CTCATGGCCTCTATCTCCCTGACTCACCTCACCCCACAGGAAGAGGAGAAGCGAAGGGTGCGCCGGGAACGAAATAAACTAGCAGCAGC
TAAATGCAGGAACCGGCGGAGGGAGCTGACCGACCGACTCCAGGCGGTGACGACAGGCCCTGGGGTCGGAGAGGGGATGTTGAGGGGAG
CTCTCTCCCCATTCTCTGCCCCCTCTCCACCTGTACCCTTATCCTGGGTTGAGAACTAGACGTTCCACACATGGAACTAGGTACTGCTG
TGGCCAGACTGGGTAGCCCAGGGCACAAACACAGACCCCCATGCACTTAAGTCAACTCCTGGTCCCCCCCCCATTTCCTGACCCCACCG
ACTACTCTCCTAGCCTTCATTTCATCCCAAGGGGCCACATGGGGCCCTTGAGGAGAGGGGCGCCCCCATCATTTCTCCCATCTGGTCTT
CAGCAAGTAACAACCCATTTTGCCTCAGTTTCTCCATCTCTGCAAACCCTCATCAAATCTCCCGCGCTCTTTCTACCCTTAACACTCTC
GAAAGCCTGTGAAATGAAATTATTCCACCTCCTGCCCTACCCACCCACACCTCTCCTGGTGCTGGTGGCATCCCCCAAAACCCACTCCC
TTCCTACGTCCTCCCTTGGTCTGAGAGTTCCCTGCTGTATGCCTGCAGGGTGAGCTGTTACTCCTTGAGGGAACAAGGGAATTGTCAAC
TTTCCTTCTCTACTTTTTCTCTTCCCCGGGAGGTAGAGAGGGAGGGGTAATAGAAGGGAACACATTAAAAACACATAACAGTGGCTCAT
GCCTGTGATCCCAGCACTTTGAGAGGCCAAGGCAGGAGGATTGCTTGAGCCCACGAGTTTGAGACCAGCCTGGGAAACATAGGGAGGAC
TTGTCTCTACCAAGAAAAAAAAAAATTAGTTGGGCATCGTGGTGCACACCACTGTCCTCCCACCTACTATGGAGGCTTTACTGCGAGGA
TCGCTTGAGCCGACCAGGTCCAGCCTCCAGTGAGCCATGATTGCACTATTGCACTCCAGCCTGCGGGACAGAGCGAGACTCTGGCTCAA
AAGCAAAACAAAACCAACCACATAACGATTGTTCATTCATTCAACAAAACCTCCTGCACACCAGAAACTTCCCCATAAACTCCCCCCTT
CTGTACCATCCTTTCGACACATGGGGGAAACTGAGGTTCCTCCAACCCAACACCCTTGCCCCAACTCCCATGGCCACCACGACTCCATC
TCAGCAGCTCTTTTTTCTCAGCCCGCGGCTGCCTTCCAGCAGCAAGTCCAAGGGAGCCATGACCCGAGTTTCCCCCTCACTCACATGCT
TTTTTCTCCTTCCTCTCTCTCTTCTGTGACCTGCCCTCCCTGGCCTCAGGACACAGATCAGTTGGAGGAAGAAAAAGCAGAGCTGGAGT
CGGAGATCGCCGAGCTCCAAAAGGAGAGGAACGTCTCGACTTTGTCCTGCTGCCCCACCAAACCGGGCTGCAAGATCCCCTACGAAGAG
GGGCCCGGGCCGGGCCCGCTGCCCGAGGTGAGAGATTTGCCGGGCTCAGCACCGGCTAAGGAAGATGGCTTCACCTCCCTCCTGCCCCC
CCCGCCACCACCGCCCCTGCCCTTCCAGACCACCCAAGACGCACCCCCCAACCTGACGCCTTCTCTCTTTACACACAGTGAAGTTCAAG
TCCTCGGCGACCCCTTCCCCGTTGTTAACCCTTCGTACACTTCTTCGTTTGTCCTCACCTGCCCGGAGCTCTCCGCGTTCGCCGGCGCC
CAACGCACCAGCGGCAGTGACCAGCCTTCCGATCCCCTGAACTCGCCCTCCCTCCTCGCTCTGTGAACTCTTTAGACACACAAAACAAA
CAAACACATGCGGGAGAGAGACTTGGAAGAGGAGGACGACCAGGAGAAGGACGAGACACACCGGAAGAGACAAAGTGGGTGTGTGGCCT
CCCTGGCTCCTCCCTCTGACCCTCTGCGCCCACTGCGCCACTGCCATCGGACAGGACGATTCCTTGTGTTTTGTCCTGCCTCTTGTTTC
TGTGCCCCGGCGAGGCCGGAGAGCTGGTGACTTTGGGGACAGGGGGTGGGAACGGGATGGACACCCCCAGCTGACTGTTGGCTCTCTGA
CGTCAACCCAAGCTCTGGCCATGCGTGGGGAGCCGGGCGGGTGACGCCCACCTTCGGCCAGTCCTGTCTGAGGATTAACGGACGGCGGT
GGGAGGTAGGCTGTGGGGTGGGCTGGAGTCCTCTCCAGACAGGCTCAACAAGGAAAAATGCCACTCCCTACCCAATGTCTCCCACACCC
ACCCTTTTTTTGCGGTGCCTAGGTTGGTTTCCCCTCCACTCCCCACCTTACCTTATTGATCCCACATTTCCATCGTGTGAGATCCTCTT
TACTCTGGGCAGAAGTGAGCCCCCCCCTTAAAGGGAATTCGATGCCCCCCTAGAATAATCTCATCCCCCCACCCGACTTCTTTTGAAAT
GTGAACGTCCTTCCTTGACTGTCTAGCCACTCCCTCCCAGAAAAACTGGCTCTGATTGGAATTTCTGGCCTCCTAAGGCTCCCCACCCC
GAAATCAGCCCCCACCCTTGTTTCTGATGACAGTGTTATCCCAAGACCCTGCCCCCTGCCAGCCGACCCTCCTGGCCTTCCTCCTTGGG
CCGCTCTGATTTCAGGCAGCAGGGGCTGCTGTGATGCCGTCCTGCTGGAGTGATTTATACTGTGAAATGAGTTGGCCAGATTGTGGGGT
GCAGCTGGCTGGCCCAGCACACCTCTGGGGGGATAATGTCCCCACTCCCGAAAGCCTTTCCTCGGTCTCCCTTCCGTCCATCCCCCTTC
TTCCTCCCCTCAACAGTGAGTTAGACTCAAGGGGGTGACAGAACCGAGAAGGGGGTGACAGTCCTCCATCCACGTGGCCTCTCTCTCTC
TCCTCAGCACCCTCAGCCCTGGCCTTTTTCTTTAAGCTCCCCCGACCAATCCCCAGCCTAGGACGCCAACTTCTCCCACCCCTTGGCCC
CTCACATCCTCTCCAGGAAGGGACTGAGGGGCTGTGACATTTTTCCGGAGAAGATTTCAGAGCTGAGGCTTTGGTACCCCCAAACCCCC
AATATTTTTGGACTGGCAGACTCAAGGGGCTCGAATCTCATGATTCCATGCCCGAGTCCGCCCATCCCTGACCATGGTTTTGGCTCTCC
CACCCCCCCCTTCCCTGCGCTTCATCTCATGAGGATTTCTTTATGAGGCAAATTTATATTTTTTAATATCGGGGGGTGGACCACGCCGC
CCTCCATCCGTGCTGCATGAAAAACATTCCACGTGCCCCTTGTCGCGCGTCTCCCATCCTGATCCCAGACCCATTCCTTAGCTATTTAT
CCCTTTCCTGGTTTCCGAAAGGCAATTATATCTATTATGTATAAGTAAATATATTATATATGGATGTGTGTGTGTCCGTGCGCGTGAGT
GTGTGAGCGCTTCTCCAGCCTCGGCCTAGCTCACGTTGGCCCTCAAAGCGAGCCGTTGAATTGGAAACTGCTTCTAGAAACTCTGGCTC
AGCCTGTCTCGGGCTGACCCTTTTCTGATCGTCTCGGCCCCTCTGATTGTTCCCGATGGTCTCTCTCCCTCTGTCTTTTCTCCTCCGCC
TGTGTCCATCTGACCGTTTTCACTTGTCTCCTTTCTGACTGTCCCTGCCAATGCTCCACCTCTCGTCTGACTCTGGGTTCGTTGGGGAC
ATGAGATTTTATTTTTTGTGAGTCACACTGAGGGATCGTAGATTTTTACAATCTGTATCTTTGACAATTCTGGGTGCGAGTGTGAGAGT
GTGAGCAGGGCTTGCTCCTGCCAACCACAATTCAATGAATCCCCGACCCCCCTACCCCATGCTCTACTTGTGGTTCTCTTTTTGTATTT
TGCATCTGACCCCGCCGCGCTGGGACAGATTGGCAATGGGCCGTCCCCTCTCCCCTTGGTTCTGCACTGTTCCCAATAAAAAGCTCTTA
AAAACGCATTCGCCAGGCTACAGTGTTTATTTCCTCCTAACACCCATTGCCTGGCTTCCTTTTAGTAAGAGAGGATGAGGTTAGATTGT
CGAAGGACTACACACACACAGAGATCATACACCCATGCACACACACACACAGACACACACCTATGCAGGAACTCCTTCTAGTCAATGGG
TCTACAGCAGGATAAAAGGAAGGCAGACAGTCAGGAGGACTTCCTGTACTCGCAGGCAGTGCCTCCTACTACAAAAAGTAAGTGGCTAC
AGCTGGGCGCAGTGGCTTGCGCCTGTAATCCCAGCATTTTGGGAGGCCAAGATGGGCGGATCACTTGAGGTCAGGAGTTTGAGACCAGC
CTTCCCAAGATGGTGAAACCCTGTCTCTACAAAAAATACAAAAATTAGCCTGGTGTGGTGGTGCACACCTGTAATCCCAGGCTACTTGA
GAGGTTGAGGCAGGAGAATCACTTTAACTTAGGAGGCAGAGGTTGCAGTGACACAAGATTGCACCACTGCACTCCAGCCTGGGTCACAA
AGTGAAACTGTCAAAAAAAAAAAAAAAAGTAAGGGGTCAGAAGGTTAGCCTGCAGGTGTGGGATCACAGAGGTCTCCTAGTGATGGAGC
TTGTATGCTCTATCGGTTAAAAACAGACGCTAAGGAGATAAACTATACACAGAAGAAGCTTAATGCCCTGTCCACAGTGGCTTGCACTT
GCAATCCCAGCTCTTTCGGAGGCCGAGGTAGGAGGCTAGGAGTTCGAGAACAGCCTGGGGCAACATAGTGAGACACCCCCCACCCCAAC
CCATCTCATTATGTTGAGAAAAAAAAAAAAAAGAGGATCTTGAGAAACTTGCACAGCAAACTACTAAAGACCACTCAGGCTAGGAATGA
GGCGTCATCTTGGATTTTTTACAAACTACAACTACAAAAAACAATTATTTTACACTGAAGATGTGTTTCCTTTTGTTTTCCATATGACC
ACTATTGCGCCCAACCCAAGACTGACTGAAGCAAATATATGACCATTTTTTTTTTTTACACATAGAGTGGAGCCATCCCCATTGCAAAA
TCCTAGCCGTCTCTTATTTGCATAACAGCAAGTCCTCTGGGAATTGAGGTGAGGGTCCAGCGGTCGCTCACACGCTTTGGTAAATCAAT
AAGCAAGGGAATTTGTCATGGTGGAATCCATTTATTAATTCACTCAAAAAAATGTGCTAAGCACCTACCCAACTGTGCTGGGAGTTGGG
GACACTATAGGGACTGAGCCTCAGGTCTTGCCCTCCTGGGGCTCACAGTCCATTCGGCGACACACACCGTTGCCAGTCAGGGACAACCT
AAAGTGGATTGGGTTTCGGAAGCCCATGGGGCTAGGGAAGCACAGCAGGGACACCTACCTTGGACTGGGAGTCGGGAAAAACTTCCTGC
CGGAGGGCACAGCTGAGCTGAGTCCTAGAGGAGGATAGAGAGTATAGAAAGAAATAGAAAAAGGCCAAGGCCAAGAAAATAGCACAGCC
ATAGATCTGTTGGTGGGGACAAGGCTTGATACATTGAGAACGCTTAGAGGGTCGGGTGCGATGGCTCATGCCTGTAATCCCAGCACTTT
GGGAGGCCGAGGTGGGTGGATCACCTGACCTCAGGCATTCAAGACCAGTCTGGCCAACGTGGCGAAACCCCGTCTCTACTAAAAAATAT
ATATATATATATACAAAAAATTAGCTAGGCATGGTGGTGCCCACCTGTCATCCCAGCTACTCGGGAGGCTGAGGCAGGAGAATCGCTTG
AACCTGGAAAGTGGACATTGCAGTGAGCTGAGATTGTGCCACTGCACTCCAGCCTGGGCAACACAGCGAGACTCTGTCTCAAAAAAAAA
AAAAAAAGAAAGAAAAAAAAGAGAAAACTCAGAGATTCGTGGAGACTGGAACCACGGGTGTGGAGAGAGGGGTTAGTACAGACCAGATT
CTGCAGGTACTATAATGACATTCCCAGGCTAAGGAGTTTAGATCTTTTCTTCACGCCACTCCGCAGCTATTGCAGGTTTTGAACACGAG
AGGGGCAGGGGCAGGTTTCTCATTTAGGAAAGACCCCTCTGGCCCCAGACTGAGCGTGACTGGAAGGCAGAGCAGTCACACTGAGCAGG
AGACCAGGGAGGAAGTGGGGTCGGGACCCTGCACGGTGGGGTGAGGAGGACTCCAGAACAGGGCCAGGGACATGGGACAGAGAGGAGGG
CAAGCATTCAGGAGACACTCGGGAGGCAGGACAGTTGGGCTTCGTCAGCAGCCGGCTTGGGAAGGAAAATACAGGGCAGTAATAAATAG
CATGGGTCAAGTGGTCTGAGTGAGGCTGGGAGTGGTGGCACGCACCTATGGTCCCAGCTACTCAGGAGGCTCACGTCGCAGGATTGCTT
GAGCCTGGAAGTTTGAGGCTGCAGTGACCTATGATCGTACCGCTGCACTCCACTTTCGGTGACAGAGTAAGACCCTGTCTCAAAAAAAA
AAAAAAAAAAAAAAAAAAAAAAAAGACACTGCCTATGAGTTAACCCTGCTCTACAAGGAGCAGTTTTTAAAGTAAAATTAAAAAAAAAA
AAAAAAAAGAAAGAAAGAAAGAAAGAAACAGAGAGAAAAAAGAAAAAAGCCTGGGTTGCCTTCGACAACAGACTTCATCTCCCTGAGCC
TCCATTTCCTCATCTGTAGAATGGGGGCTGTTAAGAGGAGTTGCAAGGCTTGTGCATGCCAGCAGTAAGTGCAGAGTGACGGTGCAATT
ATCATTACCCCCATCATCTTTATTGGGGTCAGCCTGAACCCTCGATATCCCATAATATTCACCCCCATCCTTCAAGGGTCTGCCCTAAT
GTTCCCATGACACCCGACCACCTCAGCTCTCCTTATGAGAGGGCCTCACTTTTCTATTCCCTTTGCAGCCGTTATCCCCTTTGTAGTTG
TTAATTAAGTGTGTAATTACATGATGGCTTAATGTTTGTCTCCCCCACTGGCCTGACTCCACCACGAGACCAAGGCCAGGGCTGTCACC
CACCGCTGCGTCCTCAGCAAATGCTTGTTTGATGAGTGAATGACAGATGAACAAATGGGCAAGTATTTGACTGATTAATCACGGCATGC
GTGATTAAATAAATGAGTGAGCAAACGACTGAATGAGTAAGCAATTGAAGGGGAGAGATCTAGGATAATTTCCAAACTGCCAATATCCC
AGAACTGGGTAGATGACTTTTTCTCCGTCTTTTCGAGTGGGTTTTTATACTCCCCAGCGCGAAATAACTAACGACAATCAGAGATCGGA
GTGACACACAATGAGTTTCCGATCCGGAACGCCGCACCCACCTCTCCAGGTGCAGGCTGGAGGAGCGCCTCCCGGGCAACAAGCAAAAT
AAGTCCCCTTCCATAAGTAACATTCAAGCCCCTTAGTTACTACCGCCCGAAAGGTGGAATTCAACCACTCTCTCGCTACACACCTACAG
CTTTGAAGCCTGCACCCCAGACCACTGAGGATCATCCGGGCACCACAACCTTCTTCCCGCGCAGCTATATAACGATCGCAAAAGCTTAC
CTTTTAGAGTTATGGTCGCCTCCTTTGCAAAGGTGTGAGATGGTTGACTTCAAGGGCTATAAGTTCTCCCCACTCATTTATCGTGAGAG
ATGTCCTTGTAAAACCTTGCCTTCACTTCATATAGTATGAATCATCCTCGCTCCCCAAACCGTCTCATTTACATAGCCCCACGCATTCT
GGCAAGCACCGTTTGAGCACCTGGCAAGCTGTTAAAGGGCCCACGACCCCCTTTTTCCAAAATGAAAACTCAGCTTTTTAAGACGAGAG
ATTCATGATTCCCCATTCAGAAGCGCCTTCTCGCAAAATTCCGAATTCTCTTTCTGCGGAGGAGTGGTGACTGGGTTCCTGTTACAGTC
TGATTCACCCTGGCCCCAAAGGCGCTGAGCACGCGGCTATAGGGCTGGTGCGACACCTGCTGTTCTTTTATGGAATTGCGTAGCCCGAT
GCAGCTCCCCCTTCATTCAAACCTTTATTGAGCGTCTACTGACTCCCAGGAACTGTCTCGGTGCTCGTGACGACAGACATCCTCTCCCG
ATTGAGTTTAAACACTACACATAACCAATTTCTGCACTTCCCTTTTATGGTTGGAGAAACTGAGACAGACTCAGAAACGGATAGTACCA
TATTCCGGTTAAAACAGCCCATCCACTTAGGGAAATCGAACTTGTGTTACAATTTTTATTACAAAAAAGGTGATTCTTTTTTCTTAAAT
TTTTACATATTTAGGAGCTACAGGTGCCCATTTCTACATGCATATGTTGATTGCATCGTGGTGAAACCTCGCCTTTTAGGTGTACTCAT
CACGAAAAATTATTCAATATTCTAAGTCAGTGATTCTTTCACAATACCATTCTCCTCCATGAAACTCTCCCATGACTTCCATTTCAGAG
TAAAAGTCAAAGTACTCACCCTGCTTTACAAATCCCTGTAATCCAGCCCCTTTGGTCCCTCTGACCCTGTCTCACATACTCCTCTTCCC
TTGGATATTATTGGATTTAGGCACACAGATCTCTTGCTGCACACATCGATCATGCTACCACCACAGGGACTTTGTGCTTGCCGATCCCC
TAGCCTCGATCTCCTCTTCACATGTTCCCATGGCTCATCGCCTCCCTTCACTCTCTCATCTGATCAAATGTCAATACCAAAAACTCACT
GAGTGACCAGCACTCAGTCCGGCCTTCCCTGATCCCAGTTTAAAATAGCGAGTGGCGGCCGGGCGCCGTAGCTCACGCCTGTAACCCCA
CCACTTTGGGATGCCAAAGCGGGTGGATCACGAGATCAGGAGATGGAGACCATCCTGGCTAACACGGTGAAACCTTGTCTCTACTAAAA
ATACAAAACATTAGCTGGGCGTGGTGGCGGGCGCCTGTAGTCCCAGCTACTCGGGAGCCTGAGGCAGGAGAATGCACTGAACCCGGGAG
GCGGAGCTTGCAGTCAGCTCAGATTGCTCCACTGCACTCCAGCCTGGGGGACAGAGCGAGGCTCCGTCTCAATAAATAATAAATAAATA
AAATACCTAGTGGCTGGGATCGGTGGCTCAGGGCCTGTAATCCCAGAACTTTGGGAGGCCGAGGCGGGTGGAtCACCTGAGCTCAGGAG
TTCGAGGCCAGCCTGGCCAACATGCTCAAACCCCGTCTCCACCAATGATACAAAAATTAGTCAGGCATAGTGGCTCACGCCTGTAATCC
CAGCTACTCGGGAGTTTGAGGCAGGAGAACCGCTTGAACCGGCGACGCAGAGGTTGCAGTGAGCTGAGATCGTGCCATTGTACTCCAGC
CTTGGTGAAAAGACCAAAACTCCATCTCCAAATAAAAGTAATAATAATAATAAATAAAATAAAAATTTTTAAAAAGCGACCTCTGTCAC
TCTATCCCCTTACCCTGCTACACATTTCCCCACCACTCTCATCACCACCTGCCATTTTCTCTGTCTATTTGCTTACCGTCTGTCTGCTT
CTCTACAATATCAGCACCATGAAACTATGCACTTTATTTTTGATCATTCCTGTATCTCTAGTGCCTAAAAAGTGCTTGAGAACATAGCA
GATGTTCAGTAAATGTTTGTCCAATGAATAAAAGAATATCATCACTGTCTTTTTTTCATTCTTCTTCCAGACAGGATCTTACTTTGTCA
CCCAGGCTGGAATGCAGTGGCGCAAAAACGGCTCACTGCAGCCTCGACCTCCCAACCTCAAGTGATCCTCTTGCCTCAGCCTCCCTGGG
ATGACAGGCATATACCATCACGCCCAGCTAATTTTAATTTTTTTTGTAGAAACAGGGCCCTCACTTTGTTGGCCACACTGCCCTCGAAC
TCCTGGCCTCAAGTGATGCTTCTGCCTTGGGCTCCTAAAGTCCTGGAATTTCACGCGTGAGCCACAGCACCTGGCCTCACTATCCTTCT
TTCAGCCTCAGTTTTCTCATTTGTATAACCAGACTAGTACAACTGATCTCACTGGAGAAATCATCATATAAAATTCTGACACTGGCTGA
GGCAACTGGGAGGAGCTCAGTAAACGCTCTTTCTGCTGGGCACGGTCGCTCACACATGTAATCCCAGCACTTTGGGAGGCCGAGGTGGG
TGGATCACAGGAGATTAGAAGTTCCAGACCATCTGGCAAGCATGGTAAAACCCCATCTCTACTAACAATTCAAAAAGTAGCCAGGCATG
GTGGCTCACACCTGTGATCCCAGCTACTCGGGAGGCTAAGGCAGGAGAATCCCTTGAACCCAGGAGGCTGACGTTGCAGTGAGCCAAGA
TTGTGCCACTGCACTCCATCCTGGGCCACAGAGCAAGACTCTGTCAAAAAAAAAAAAAAAGTTTTTTTTTTTTGGCTGGAATTACAGGC
GCCTGCCCCCACACCTGGCTAATTTTTGTTTTTTGTTTTTTTAGGAGACACCCGGTTTCACCATGTTCACCACACTGGTCTTGAACTCC
TGACCTCACCTAATCCAACTCCCTCAGCTTCCCAAAGTGCTGAGATTACAGGCGGGAGCCACTACACCTGGCCAATAAAGGCCGTTTCA
GTCTTCAATCTGTTTTGAGCTTGGAGGCTTTAGTCATTCCCAGACCCAAAATCTCAATCAGACCCTCTTCCACCACTTTTTGTGATAGA
TCAATAAACATTTTGTCTTATGGGAAGTTTAACTAAGAGTATCTTTAGAAACTTTTGGACAGGCGCTGTAATCCCAGCACTTTGGGAGG
CCGAGATGAGCGGATAGCTTCAGCCCAGGAGTTAGAGACAAGCCTGGGCAACATAGTGAGACTCTGTCTCAAAAAAAAAAAAAAGAAAC
AAAGGAAAAGAAAAAAAGAAAAAAAAAGTATTCCCTCTGCTTGGCAAATCCAGATTCAAGATATATCCCCTAAACCCTCTTTGTTTTAA
TTAGATAGTTGCTGCTAGCCACCTCTGTATACAAATTCTGAGGATGTAAGGACTCCTTGGAACACCGTTATCTGTCTCCTAATATGTGA
CGTGTGTATCACGAACAAGTCAGTTTTTTTTTTTTTTGTTTCTTTTTTTTGGTTTTTTGTTTTGAGACGCAGTCTCGCTCTGTCGCCCA
GGCTGGAGTGTAGTGCCGCGATCTTGGCTCACTGCAAGCTCTGCCTCCCAGGTTCACCCCATTCTCCTGCCTCAGCCTCCCTAGTAGCT
GGAACTACAGGCCCCCGCCACCACTCCCCGCTAATTTTTTTGTATTTTTAGTAGAGACGGGGTTTCACCGTGTTAGCCACCATGGTCTC
GATCTCCTGACCTCATGATCTGCCCGCCTCGGCCTCCCAAAGTGCTGGGATTACAGGCGTAAGCCACCGAGCCCGGCTTTTTTTTGAGA
TGGAGTTTCCCTCTTGTTGCTCAGGCTGGAGTGCAGTGGTGTGATCTCGGCTCACTGCAACTTCCGCCTCCCGGGTTCAAGCGATTTTC
CTGCCTCAGCCTCCCAAGTAGCTCCGATTACAGGCATGCACCACCACGCCCGGCCAATTTTGTATTTTTAGTAGAGACATGGTTTCTCC
ATGTTGGTCAGGCTGGTCTCCAACTCCCGACCTCAGGTGATCCACCTGCCTTCCCCTCCTAAAGTGCTGAGATTACAGGCGTGAGCCAC
TGCGCCTGGCTGCACAAACATTTTAAACGTCAATATTTTTGTGTTTATTTTTACTATTGTCTTATATTTCTGGCAAGCAATACTGGTTT
CTGGTGGCAAGGTAACCTTTCTTCTTAAAATACATTTGTTATTATATTATTAAATATTAAACCAATTTTCAAGAAAAATATTAGATAAA
TACCACAACAGGTTGTAGAAAGATGAAGGTCATGTGGGGAATGACTGAGGTTTGGGAAACAATATTATAACTCCTTACTGTGCATTTAT
TATATGCCAGGCCCAATGCCAAGGGCTTTACATGTACGTATAGTTATGGTTGACAGTTTTCTTCTTTTTCTTTTCTTTTTTTTTGAAAC
AGGGTCTTGCTCTGTCGCCCTGTCTGGAGTGCAGTGACGCAATCTCAGCTCACTACAACCTCCACCTCCTTAGTTCAAGTGATTCTCCT
GCCTCAGCCTCCCGTGTAGCTGGTCTTACAGGTGCCCACCACCACATCCGGTATTTTTAGTAGGGACGAGGTTTTACCATCTTGCCGAG
GCTTGTCTTGAACTCCTGACCTCAGGTGATCCACCTTCCTTGGCCTCCCAAAGGCTGGGATTACAGGTGTGAGCCACTGCACCTGGCCT
CTTTTTTTCTTACTTAATTTTTTTGTAGAGATGAGGTCTATGTTGCCCAAACTTGTCTCCAACTCCTGGCCTTAAGTGATCCTCCCTCC
TTGCCCTTCCAAAGTCCTGCGATTGCACACGTGAGCCATCGTGCCCGGCCTAATAAGTTTTCCATTTGAGGAAACTCAGGCTTTGAGAG
ATGAAGTCATATGCTCAAGGTCATACAGCGAAGGGCTAGTAGAGCCACGATTCAAACCCAAGTCTGTGGATCTTTAGACCTCAGTGTTC
TCCCAGTCTCCCCACGGGTCCCTTTGCTTCATCCTTTCCGAGTTTAGTTGTCTTTTTTGTTGTTGTTCTTGTTGTTGCTGTTTTTTGAT
AGAGTCTCGCCCTGTTGCCCAGGCTGGAGTCCAGCGGCATGATCCTGGCTCACTGCAACCTCTGCCTCCTGGGTTCAAGTGATTCTCCT
GCCTCAGGCTCCCGAGTAGCTGGGATTACAGGTATGCGCCGCCACACCCAGCTAATTTTTGTATTTTTAGTAGAGATGGAGTTTCATCA
TGTTGGTCAGCCTGGTCTGGAACTCCTGACCTCAGTGATCTGCCCACCTTGCCCTCCCAAAGTGCTGGGATTACAGGCGTGAGCCACTA
CGCCCAGTATGTACTTTTTTTGTGTTTTGTTTTTGTTTTTGTTTAGAGACAGCGTCTCCCTCTGTAACTCACGCTGAAGTGCAGTGGTG
TGATCATAGCTCTCTGTAACCTCGAACTCCTGGCCTCAACCAATTTTCCTCTCTTAGCCTCCCAAGTACCTGGGACTACAGGCATGCAC
CACCACGCCCGCATAAATTTTTTTTTTTTTTAGAGACGCGGGTCTTGTTATGTTGCTCAGCCTGGTCTCGAACTTTTCCCCTCAACTGA
TCTTCCCACATTGGCCTCCCCAACTCTTGGGATCACAGATCTGAGTCATCATGCCAGGCCTCAGATTAGTGTTGAAACTGGAAGTCCAG
GGAGGCCCTCTATTTGTTCCCGACGCAATCAGGGTGGGGAGACGTGGGTGGATGAGAGAAGTTTGTGAGGCAGGATAGACACAGCATTG
TGACTGATTGTTTGTGCGAGGTGAGTGTGTAGAAGTCCAGAGCCATACCTGGGTGTCTGGCCTGGTGACACCGTGGGCAGTCAAAACAT
CTCTGAGTTGGAAATCAGGTAGGATCAAGAACACCTTTGGTTAGATTTAGGGTGGAAGGGGCATCCAGAGGCCGTTGGACACGTGGGTT
TGGGCTCAGTTGAGGTGGCTGCACAGTAGGCAGGGTGTGGGATCCATTAGTGATGTCTGCCTTGAGCACACGCATGGGAAGTGGCTCCC
AAGGCTTGGCTAACCCTGGAATGGTGGGTCTGTCAGCATCTTCCCCTCAATCATTTCTTTCTTTTTTTTTTTTTTGAGACAGAATCTCG
CTCTGTCGCCGAGGCTGGAGTGCAGTCGCGCAATCTTCCCTCACTACAACCTCCATCTCCCACATTCAAGCGACTCTCATGCCTCAGCC
TCCCGACTACCTGTGATTACACGCATGCACCACCACACCCGGCTAAATTTTGTATTTTTAGTAGAGATGACCTTTCACCATGTTGGCCA
GGCTGGTCTTGAACTTCTGGTCTCAACTCATCCGCCCTCCTCGGCCTCCCAAAGTGCTGCCATTACAGGCGTGAGCCACTGCGCCCGGC
CTCCCCTCAATCATTTCATAACCTACACAGATGGCAGAG

| | |
|---|---|
| HUMAN SEQUENCE - mRNA | (SEQ ID NO: 11) |

CATTCATAAGACTCACAGCTACGGCCACGGCAGGGACACGCGGAACCAAGACTTGGAAACTTGATTGTTCTCGTTCTTCTTCCCGGTTA
TGAAATTTCATTAATCTTTTTTTTTTCCCCCGAGAAAGTTTTTGGAAACATTCTTCCAGATATTTCTTCATTTTCTTTTGGACGACCCA
CTTACTTTTTTTGGTCTTCTTTATTACTCCCCTCCCCCCCTCGCACCCCCCGGACCCGTCGAGGACACCGTAGCTGAAGCTGATTCTGT
ACAGCGGGACAGCGCTTTCTCCCCCTGGGGGAGCAACCCCTCCCTCGCCCCTGGGTCCTACGGAGCCTGCACTTTCAAGAGCTACAGCG
GCATCCTGTGGGGGCCTGGGCACCGCAGCAACACTCCACAGAAACTTTGCCATTGTTGGAACGGGACGTTGCTCCTTCCCCGAGCTTCC
CCGGACACCGTACTTTGACCACTCGCTCAGCTCACCGGGGACTCCCACGGCTCACCCCGGACTTGCACCTTACTTCCCCAACCCGGCCA
TAGCCTTGGCTTCCCGGCCACCTCACCCTGGTCACAGGGGCCCCCCTGTGCCCAGGGAAATGTTTCAGGCTTTCCCCGGAGACTACGAC
TCCGGCTCCCGGTCCAGCTCCTCACCCTCTGCCGAGTCTCAATATCTGTCTTCGGTGGACTCCTTCGCCAGTCCACCCACCGCCGCGGC
CTCCCAGGAGTGCGCCGGTCTCGGGGAAATGCCCGGTTCCTTCGTGCCCACGGTCACCGCGATCACAACCAGCCAGGACCTCCAGTGGC
TTGTGCAACCCACCCTCATCTCTTCCATGGCCCAGTCCCAGGGCCACCCACTGGCCTCCCAGCCCCCGGTCGTCGACCCCTACGACATG
CCGGGAACCAGCTACTCCACACCAGGCATGAGTGGCTACAGCAGTGGCGGAGCGAGTGGCAGTGGTGGGCCTTCCACCACCGGAACTAC
CAGTGGGCCTGGGCCTGCCCGCCCAGCCCGAGCCCCCCCTACGAGACCCCGAGACGAGACCCTCACCCCAGAGGAAGAGGAGAAGCGAA
GGGTGCGCCGGGAACGAAATAAACTAGCAGCAGCTAAATGCAGGAACCGGCGGAGGGAGCTGACCCACCCACTCCAGGCGGAGACAGAT
CAGTTGGAGGAAGAAAAAGCAGAGCTGGAGTCGGAGATCGCCGAGCTCCAAAAGGAGAAGGAACGTCTGGAGTTTGTGCTGGTGGCCCA
CAAACCGGGCTGCAAGATCCCCTACGAAGAGGGGCCCGGGCCGGCCCCGCTGCCCGACGTGAGAGATTTGCCGGGCTCAGCACCGGCTA
AGGAAGATCGCTTCACCTGGCTGCTGCCCCCCCCGCCACCACCGCCCCTGCCCTTCCAGACCAGCCAAGACGCACCCCCCAACCTGACG
GCTTCTCTCTTTACACACAGTGAAGTTCAAGTCCTCCCCCACCCCTTCCCCGTTGTTAACCCTTCGTACACTTCTTCGTTTGTCCTCAC
CTGCCCGGAGGTCTCCGCGTTCGCCGGCGCCCAACGCACCAGCGGCAGTGACCAGCCTTCCGATCCCCTGAACTCCCCCTCCCTCCTCG
CTCGGTGAACTCTTTAGACACACAAAACAAACAAACACATGGGGGAGAGAGACTTGGAAGAGGAGGAGGAGGAGGAGAAGGAGGAGAGA
GAGGGGAAGAGACAAAGTGGGTGTGTGGCCTCCCTGGCTCCTCCGTCTCACCCTCTGCGGCCACTGCGCCACTGCCATCGGACAGGAGG
ATTCCTTGTGTTTTCTCCTCCCTCTTGTTTCTGTGCCCCGGCGAGGCCGGAGAGCTGGTGACTTTGGGGACAGGGGGTGGGAAGGGGAT
GGACACCCCCAGCTGACTGTTGGCTCTCTGACGTCAACCCAAGCTCTGGGGATGGGTGGGGAGGGGGGCGGGTGACGCCCACCTTCGGG
CAGTCCTGTCTGAGGATGAAGGCACGGGGGTGGGAGGTAGGCTGTGGGGTGGCCTGGAGTCCTCTCCAGAGAGGCTCAACAAGGAAAAA
TGCCACTCCCTACCCAATGTCTCCCACACCCACCCTTTTTTTGGGGTGCCCAGGTTGGTTTCCCCTGCACTCCCGACCTTAGCTTATTG
ATCCCACATTTCCATGGTGTGAGATCCTCTTTACTCTGGGCAGAAGTGAGCCCCCCCTTAAAGGGAATTCGATGCCCCCCTAGAATAAT
CTCATCCCCCCACCCGACTTCTTTTGAAATGTGAACGTCCTTCCTTGACTGTCTAGCCACTCCCTCCCAGAAAAACTGCCTCTGATTGG
AATTTCTGGCCTCCTAAGGCTCCCCACCCCGAAATCAGCCCCCAGCCTTGTTTCTGATGACAGTGTTATCCCAAGACCCTGCCCCCTGC
CAGCCGACCCTCCTGGCCTTCCTCGTTGGGCCGCTCTGATTTCAGGCAGCACGGGCTGCTGTGATGCCGTCCTGCTGGAGTGATTTATA
CTGTGAAATGAGTTGGCCAGATTGTGGGGTGCAGCTGGGTGGGGCAGCACACCTCTGGGGGGATAATGTCCCCACTCCCGAAAGCCTTT
CCTCGGTCTCCCTTCCGTCCATCCCCCTTCTTCCTCCCCTCAACAGTGAGTTAGACTCAAGGGGGTGACAGAACCGAGAAGGGGGTGAC
AGTCCTCCATCCACGTGGCCTCTCTCTCTCTCCTCAGGACCCTCAGCCCTGGCCTTTTTCTTTAAGGTCCCCCGACCAATCCCCAGCCT
AGGACGCCAACTTCTCCCACCCCTTGGCCCCTCACATCCTCTCCAGGAAGGCAGTGAGGGGCTGTGACATTTTTCCGGAGAAGATTTCA
GAGCTGAGGCTTTGGTACCCCCAAACCCCCAATATTTTTGGACTGGCAGACTCAAGGGGCTGGAATCTCATGATTCCATGCCCGAGTCC
GCCCATCCCTGACCATGGTTTTGGCTCTCCCACCCCGCCGTTCCCTGCGCTTCATCTCATGAGGATTTCTTTATGAGGCAAATTTATAT
TTTTTAATATCGGGGGGTGGACCACGCCGCCCTCCATCCGTGCTGCATGAAAAACATTCCACGTGCCCCTTGTCGCGCGTCTCCCATCC
TGATCCCAGACCCATTCCTTAGCTATTTATCCCTTTCCTGGTTTCCGAAAGGCAATTATATCTATTATGTATAAGTAAATATATTATAT
ATGGATGTGTGTGTGTGCGTGCGCGTGAGTGTGTGAGCGCTTCTGCAGCCTCGGCCTAGGTCACGTTGGCCCTCAAAGCGAGCCGTTGA
ATTGGAAACTGCTTCTAGAAACTCTGGCTCAGCCTGTCTCGGGCTGACCCTTTTCTGATCGTCTCGGCCCCTCTGATTGTTCCCGATGG
TCTCTCTCCCTCTGTCTTTTCTCCTCCGCCTGTGTCCATCTGACCGTTTTCACTTGTCTCCTTTCTGACTGTCCCTGCCAATGCTCCAG
CTGTCGTCTGACTCTGGGTTCGTTGGGGACATGAGATTTTATTTTTTGTGAGTGAGACTGAGGGATCGTAGATTTTTACAATCTGTATC
TTTGACAATTCTGGGTGCGAGTGTGAGAGTGTGAGCAGGGCTTGCTCCTGCCAACCACAATTCAATGAATCCCCGACCCCCCTACCCCA
TGCTGTACTTGTGGTTCTCTTTTTGTATTTTGCATCTGACCCCGGGGGGCTGGGACAGATTGGCAATGGGCCGTCCCCTCTCCCCTTGG
TTCTGCACTGTTGCCAATAAAAAGCTCTTAAAAACGC

| | |
|---|---|
| HUMAN SEQUENCE - CODING | (SEQ ID NO: 12) |

ATGTTTCAGGCTTTCCCCGGAGACTACGACTCCGGCTCCCGGTGCAGCTCCTCACCCTCTGCCGAGTCTCAATATCTGTCTTCGGTGGA
CTCCTTCGGCAGTCCACCCACCGCCGCGGCCTCCCAGGAGTGCGCCGGTCTCGGGGAAATGCCCGGTTCCTTCGTGCCCACGGTCACCG
CGATCACAACCAGCCAGGACCTCCAGTGGCTTGTGCAACCCACCCTCATCTCTTCCATGCCCCAGTCCCAGGGGCAGCCACTGGCCTCC
CAGCCCCCGGTCGTCGACCCCTACGACATGCCGGGAACCAGCTACTCCACACCAGGCATGAGTGGCTACAGCAGTGGCGGAGCGAGTGG
CAGTGGTGGGCCTTCCACCAGCGGAACTACCAGTGGGCCTGGGCCTGCCCGCCCAGCCCGAGCCCGGCCTAGGAGACCCCGAGAGGAGA
CGCTCACCCCAGAGGAAGAGGAGAAGCGAAGGGTGCGCCGGGAACGAAATAAACTAGCAGCAGCTAAATGCAGGAACCGGCGGAGGGAG
CTGACCGACCGACTCCAGGCGGAGACAGATCAGTTGGAGGAAGAAAAAGCAGAGCTGGAGTCGGAGATCGCCGAGCTCCAAAAGGAGAA
GGAACGTCTGGAGTTTGTGCTGGTGGCCCACAAACCGGGCTGCAAGATCCCCTACGAAGAGGGGCCCGGGCCGGGCCCGCTGGCGGAGG
TGAGAGATTTGCCGGGCTCAGCACCGGCTAAGGAAGATGCCTTCAGCTGGCTGCTGCCGCCCCCGCCACCACCGCCCCTGCCCTTCCAG
ACCAGCCAAGACGCACCCCCCAACCTGACGGCTTCTCTCTTTACACACAGTGAAGTTCAAGTCCTCGGCGACCCCTTCCCCGTTGTTAA
CCCTTCGTACACTTCTTCGTTTGTCCTCACCTGCCCGGAGGTCTCCGCGTTCGCCGGCGCCCAACGCACCAGCGGCAGTGACCAGCCTT
CCGATCCCCTGAACTCGCCCTCCCTCCTCGCTCGGTGA

TABLE 3
(mouse gene: Cend1; human gene: CCND1)
Mouse genomic sequence (SEQ ID NO: 13)
Mouse mRNA sequence (SEQ ID NO: 14)
Mouse coding sequence (SEQ ID NO: 15)
Human genomic sequence (SEQ ID NO: 16)
Human mRNA sequence (SEQ ID NO: 17)
Human coding sequence (SEQ ID NO: 18)

| | |
|---|---|
| MOUSE SEQUENCE - GENOMIC | (SEQ ID NO: 13) |

CACGGGATCCAAAACCCTCTCCTGACTTCTGCAAGCGCTGCACACACATGGTACATACACATGCACACGCACGTGCGTAAACATGCTGG
CTTTCTGATCCTACGGCTTCTAATTTAGCACTAACACTTAGACTTTTTATTCCCTGTTGCTGCCATGACTGACCCCAGACCCTGCCTTT
CACGCTTCTCCTTGCTTTTCAGACATGGGACAGTTCTGCGGTCTCTTTTGCAAATTGAGACCTGTGCTCACCTAGCACACATGTGGCCT
TGGGTTCCATCCCCAGAACTGAGTGAATGAGGCATGGTGGCTCACCCTGTGATTCCAATATTCAGAATGTGGAGACAGTAGAATGAAGA
GTTCCAGTTTCAGGTACACCAAACCTTGTACCAGAAAGAAGAGGGGGAAGAGAAGGAAGAGGAGGGAGAAATCTAGCAAAAGATGGACG
AAGCATACAGTGACTTTGAAGATGTGTTAACTTTCCCTTGCTGTAAGGAAATACCTGCGGTAATTAACTTATAAAGAACAGAGGTTATG
TGAACTCACAGTTTGGGGATTTTAGTCCAGAACAGGACTCTATTCTACTTCGAGTCTCTAATGCAGATGATGAGAGCGAAATGCTGAGC
AAACAGAACTGAACTGCCCTGTACTACATGGCAAATCAGAGGAAGAGTAGTCCCATCGTCCTCTCTGAGGCCACCTTCAGTAACCTAAG
GACTGCCAGGCTGTGCTGTTTGTTTGTTCTCTGTCTCTAGGGTGTTTGTTTTCGGGTTTGTTTTGGTTTTGTTGGTGGTAGGTTTTGTT
CATTTGCTTGTTTGTTTTTGATATAGAATCATGCTATGCAGCCCAGGCTGTTCTCAAACTTGTAATCCTCCTGTCTTCGCCCCCTGACT
ACTAGGATCACAGTATGCGCCACCACACCTGGCTCTAGAATCCATCTCTTGGAAGTTCTACCACCTCCCAATAAAGCCACCCTGAGAAT
GAAGCCTTGGACAACCAGAGAATATTGCTGAGCCAAACTACAGCAAGAAGGCTGACCAGAAGGCTCTGCCAAATTCACAAGCCACTGGC
CCATCTGCAATACATGCCATCTGTAAAGGAGTCACTTCTCCCCAGGGTGACAGATTCAATGCAACCTCAATCAAAACCCTAGCTGTGGT
GGGTTTGTGTGTGTGTGTGTGTCTGTGTGTGTTTATGTGTGTGTCTCTCTATGCATATCTGTGTGTGTGTTGGGGGTGCTAACCTGTGT
CTGTGCATGCATGCACAGTGTCTGAGAAAGAGGGTCCAAGATGCCGGCCACTGTAGGCCATGTTACTGTTCGCATGGGACAGGCCTTAA
AGAAGAGCTGCAAAAGAGCCTTGTACCCACCTCCTCCAATGAGACCCACCCCAGTCTGCTTTCTCAGTTAATCTCTTCCTCTCTCAACT
TGAAGCCTGTCTGGGAATTTGCTGTTGAGTAGATTATAGGCTTGGCCTCAGCTGGTCCACTAAAAGCAGGAGGTGAGGGCCCAGGCCTG
AGGAAGCATTGGGGAGGCAGATCCCAGGCCTAAGTTTGTTGGAGACACATTCTCCAGGACCACAAATAGCTGACATGCTGTTACCAGAT
TGTCATCCCAGTACCTGCAAAGTGGGAGAACCAGAATCAGGTGTTCAAGGCCAGCCTCTCAAAAAACAAAACTGAAGTAAGTACATCAA
CTCTACTCTGCCTTTTAAGAGTTAAAAAGGGGCCTGGAGAGATGACTCAGCAGTTAAGAGCACTGGCTGTTCATCCAGAAGAACCTAGG
CTCAATTCCCAGCACCCATATGGCAGCTCACAACTGTCCATGACTTCAGTTCCATAATTCCAGGGGATTTGTCATGTTCACACAAACAT
ACATGCAGGCAAAACAGCAATGCACATAAAAAAAAATAAATCATAAAACAAACAACAAAGAGTTAAAAGGACAGGAGAAGAGGAACAAG
AGAACGTTCATGTGGCCAGGTTTTCTTTTCCCCAATTTAAAAGTTTCTTCTGAGGTTTACATCTCACACCCATAAAGAGCTCAGAAGTC
AACAAAGCGAGGAGGCTTGATTCATCATGAAGTCATTTCAAAATTTCTCTTTCTGAATATAAACCAAAATATTTAATTAATTCCACATA
CCACAGGAAGTACATTACAGCCTTCAAATTCACTCTGCTTAACAGTAAACTCGTGAACGAATGCACAGAACAGACCCCAGTCACCCACC
AGCACTATGCTAAATGTCCTATGCACATTACCTGAATTACTTCTGGCCATGGCCTCGCAGAGGAAATCATCCCACTGCCCAGCAGAGAT
GAGGGCATCTTTGCCCACAGCCTCTCCAATGCCAGAGAGCACAGCTCTGTACGTTGCCTCCTGTAGTTACAGAAATGTTTTCCAACTTC
CTGGCATCCTGAAAAGAAGTGGCTTGCCTTAAAGTGACTTTTCTTTTTCCTTTTCACAAACAGTGCAGGATGGCCTCCTGTGCCAATGA
CAGGTATGTGCTACCTCATCTAGTTCTGGTAGTACGTGGATTTTCTCTCTCTCTCTCTCTCTCTCTCTCTCTCTCTCTCTGTGTGTGTG
TGTGTGAGAGAGAGAGAGAGAGAGAGAGAGAGAGAAAGAGAGAGAGTGAGAGAGAGAGTGTTTGTGTACCTGTTCTTTCTTGTGTATGT
GTGTACCTGCTCTTTCTTGTGTGCACATATGTGCACCTGTTCTTTCCTCTGTGTGTATGTATCAGCTCTTTCTTGGGGGGGGGTGTATG
TCCCCCTGCTTCTGCCTTACAAGGGCTAGTATCTCAAGCAATGAGTTGGTGGAGTTACAGATGCCTGTGTCCAGCTCTCTATGGATTGT
TAAGCTGCTAACCTCAGCACACTGGTTTTCTGCCCTGTCAAATGCCTGCGATGACAGCATCCACCTGGTAAGCTGGAAGGTGGGGTCTG
CTCCTCCATCCCACCTGACAGGGAATGATTTCCTTAGATAGGCTGCGGCTGGAACTCACCTGATCTAGAAGTACACTGGGGGCACACAG
GAGCCATGGAGCCCCTAAGGCCCACCAGCAGCTTCTGTCCTATCCCTTCTGGCAAAGGGGCCATCTGTCAAAGGAGGGGTTGAAAGATG
GCCCAGAAGAGGTCTCCTCAAATCTCTGACCCTTACAGGACTCTGATCATAAATGATACAGTCTCAGTAGGCCTATCCTGGCTTCATTT
CTTCTGCTGTGATAAAATGTCATTGGGGGAAAAAAAAAAAAGCAGCTTAGGGAGAAAGGGCTTATTCTGGCTCGGAACTCCAGGTTTCC
AGTCCATTACTACATGAAAGGCAGTGTGGCAGGAACTTGAAGCACATGGTCACATCATATCCTCCCAGAGCAGTGAGATATGAATCCAG
GGGTGGGGACTGGAATGATGGCTCAGGGGTTAAGAGCATCGGCTACTCTTCCAGAGGATGCCGATTAAAACTGTCTGTAACTCCAGTTC
CAGGGGATCTGACACCCTCACGCAGACATACTTCAAATAGACATACATGTGGGCAAACACCAATGTACTTAAAATAAAAATAAATAAAT
TATAATAAATAAATAGGTGCTTAGTTACTTTTCCCACCTTTAATCAGTCCAAGACCCAAGCCTAGGGAATGTGTCACCCACTCCCACAC
TGGGTCCTCTCACATCAGCTAAGATAGCCAAGACACTACCCACAGCCGTACCCATAGGGCTTCTTGAGACTCTCTAGCAACGTGAATCG
AGCTTGTGTCAAGTTGACAATTGAAACTATTATGGTGCTTAACTGCTCCCTGATGAACAGGATGTGGCAGAGACAGGCATCCCATTGGT
TAATGTCAGAAGTGTCATCCCTCCGTGGACTGAATTTTTGCCTTTAACTTATATTTATTTGCTTGTTTGTTTATTAGTGTTTGTATCTA
TGTGCATGTGTGAGTGCGGCTTCCCTCAGAGTCCAGAAGAATGTGTAGAATCCCTCTAGAGCTGGAGTAGGGGGCTGCCGGGTATGGGT
GCTGGAAATCAAACCCGGTCCTCTGGAAAAATCAGCAACCACTCTGACCTGTTAAATCATCTACCTAACCTCATCCTTGCTCATGTTTT
AAAAATAGTTATAGTGGCCAGGTGGTGGTGGCGCTCGCCTTTAATCCCAGCACTTGGGAGGCATTGGCAGGAGGATTTCTGAGTTCGAG
GTCAGCCAAAGTGAGTTCCAAGACAGCCAGCAGGGCTATACAGAGAAACCCTGTCTCAAAGAACCAAAAATAAATGAAATAATTATAGT
GGGTTTTTGGACATTCTCTGTATGCTAAGCACCTTCCAGATGGGCTTCCTGACAAGAGATCCGTGGCCTGACCCACACTGATTTGAAAT
AATGATGAAAATTCAACTTTCAGAAGCAAAGGCTGTAGTTCCTCTCTGGCTTCTACCACAGTCAGTAACCAGTCCAGTTCCTTAGAGAG
TCAGTCACCTTAGTACACATTCCAGCGGAGCCAAGACAGTATAGTGCTCCTTATAAACATGGAAGGGCAGTTCCTACCCAGCGAGTCAC
TGCGGGCTTGGTGCCTCTACTGAAGCGCAGGCTCAACCACCGTTCACCGCGGGGAGCGTCCTCAGGCTCTCGCGAGCCACTGCTGCGCG
TCGCGTCCAAGGTTTACGGTAAGCGCGGCGCTCAGGACGACCACGTGGCGGCGAACACCGGCCGGCGGGAATGCGCGGGAATGCGCGGT
GCGGCCTCGCGCGCTCCCCAAGCCGTGACCCCGGAGTTCCCGGCGCGCGTTTGTGGGACCGCGGAGGCTAGTGCCATGGGTCCGCCCGC
GCGTCCAAGCCACCAAGCACCACAGGGAGCAGGGGCCCCATGGAGGGCTCAGCACGTGAGGCTAGACAACCTTTTCCAAAACCTGGGGC
TAGGTTTGCCCTCCCTGTTTGGGCTCCATCACAGATCTGGAACCCGCTTAGTCCCCATTCTAAAGCCCCCACTGATCTGGATTAAGAAT
GGAGATGCCTGGTTTTGCAGGGAAGCAGGTCAAAGGGCTCTGCGCCCTGACCGCCCTTCGGAGTGCTGAGGGACCAGTGCCAGGGGCTG
AAAGACCCCAGTTCCAGCAGCTGCGCCCTGGGATCGCATGAGGGTTCGCTCACCACGATTGAGCCACGTGGTCCTGCCTGGCCACTGGT
GGCTCACCTTGGCCCGAGTGCCCCCTCCCCACCATTTAGTCTGGGAAGGGCCGAGGGCCAAAGCCCAAAAGGACCCTAGGGTTCTTCAG
AGCACCTGTCATTCGTTCACAGACCTGGACACCGAGCACACATCCCTCCTTCAAATTGCATCTCTACCCTTGGGGGTCCCTGGAGCCAG
GCTTCCCCCTCCACAGCTCATTCCACGGCCCGCGTCTATTCGCTCTGCGTCCTGGGATTTAGGGACTGAAACTTATTTCCTAAGGCACG
CTTCGGAAGATACGATGCGACGCTCCCAGGTGTCCACAGCCGATGAGCTCAGCATACACACCTTAGCCGAAGGGTGCCTGAAATCCCCT
CAGGGTAACCTAGGCGGAGCAGCCGTGTAGCACGTGGGCTGCCACGCGCGCCCCAAAACGCCTTCTGGGTGAGGAGAGGGAAGCCGTAA
TGCCTCCGGAACCTTGGGGTCCATATTCGGAAGTGTTTTCTCTGGGCGACTTGAAGGCTAAATTAACAATGGCTAGCTGGAGCAGAAAC
ACCCAAGTCCTTTCAAGTTGCCCCCAGGTATGCGGCTGCAGGTGACCCCACCTAGGTGCGTCCGCTCTTCTCCAGGACCGGCTACAGCC
AGAGGCTCAGTATTGCCGCCCAGCCCCGCACCCCTAACCCGACCCCCGCCCTTAGTCGCGAGGGTTCCTCAATGAACGCGCTCCCTCCC
ACTTCTCAATGAGTTCCCACAGCCAGGGATAGTGGCAAACGCAAGACTAAATCTCCGCTCTCTTTGGAACCTTGGCTCCAGTCAGGTCG
GGGGTAGGGGTAGGGTTCGAGGAGGCAGGAAATCCGAAACCGGATTAAAGTTACTTTGAGATTTTCTTTCCAAATAACGTGCTTCCCTC
TCGGCTGGGCAAGTGGCCTTTTGTCTCCAAAAGGTCACTGACAAATAAGCGCAGAACACTTTAAGCCCAGAGCGGACAGTTCCTGCCAC
GAGATTTTTTTTAAAGCCAATGTAAAGTTAAAATTCCAAAACAAAGTGGGTGTTTGTGTTTAAGTTACTATTTCGCTGGAAAATAAAAA
TCCAAGTTCTTTCTCTGAGATTCCTAAAGCAGTTGTCCCGGAAAGAACTCTGAATTGCAGCCCACCTCCTGGGCTCGGGATTCGGACAA
GCAGGGTTTCCAGCATGGAGGATGGGCGCTCCCCCCCGCGTCCCAAATTCCTCCGCCTCCCCTACCGGTTCCTGGACGGCTCTACACGC
CCGCCAACCGGACTTGCACTTTTGCTCCTCTCCCTGAGCGCAGAGCTCAACGAAGTTCCTCGTGCAGATCTGCCCGGTCCGCCTAGTAA
CAGCACTGAGTCCGGATTGGCTCATGCAAATTTCAGTTTCCTGGTCTTTCCCGCGGTGGCGGTGGTGCGCGGTGCCTGCGGGTCGCTAG
CTCGTGCTGCAGGGTCCGCGACCCCCTTTATGCCCCGCTGCCCTTCGTGGCGCACCCTCGCTAGGCTAGCCTGCTCAGTTCGCAGGCAC
TCACCCGCGCGTGATAGCGCGCAGCCCTATAAATCATGCTTACTGCCGCCCGGTAGGGATTTTATGAATGAAAAGGCAGCCTGGCCGCC
CTCGTGCTTACGCTAAGGCTCCCAGGCTTGGCCCGCCCTGTCCCCAGAGCAAGCGTGCGCACTCTGCCCGGGCCACCCACCACCTTCGG
AGCTACAGTGGAATCACTGTCCCGAAGGGTCCGGACTTTAGGGACCCCAATGTCGGGGGAGGGGGAGCGGAAGGCATGAGCTCGGAAGG
TGAGGGGTTTCCGTCTTGTGTGCCGCCTGGACGCCGTGCGCTCCTTTACCAGTTTCTTCTGGGACACGAAGGTATTCCGTGGGAAATGT
GTGTGAATAGTTCGCCTAGCTTGAAGTCGGGTATTGTGAAAACGTTTCCAAAACTAGGGAACTAAATAATGGACGAAGGTGGTGGAACC
GCTTTATTCTAAAAATATTTTATTTGAATCTATAAATTATAAACTTAGGACTCCATTCAGTTAACCACGACCATCTATAGATTCTCTTT
AAATATCACCTTATCGGCTCACAAGTTTATCTTGATTCTCACCAGCCGCCCCCCCCCCCCAACTCAAGACTGCAATTCTAAACGTGGAG
AAACACCACCACCCTCAACGAAGCCAATCAAGAAGCTTCCGGTGGTCTGGTTCCTGGAAGGGCGACTAATAACTTGCAGCGATTTACTT
TTCTTAATTAAAAAATAAATTAGGAAGGAGCCTATCGTGTCTCAACCTTTTCTTCAACGGTTTATTTTTCTTGGGCAAACCGCCTGCAG
TGGAGCAGGGGGATACTGGGGGACCCTGGCCAGGATAAACCGGTCACTGTATGAAGACAAATCTCAGATCCCACCCCACCCCCCAGCGA
GGAGGAATACATGAAATAATGGCCACCATCTTGAGCTGTTGCTGGAATTTTCGGGGTTTTATTTTATTTTTGACCGAGCGCATGCTAGG
CTGGGGATCCTTTAAAGTTCAGATACCCCTCTGGCCCTTTGCAACCACCCCAGTGCGCGAGGATGGAGCCTGCACGAGAGCTTAGGGCT
CGTCTGGCATCTTCGGGTGTTACACAGTTCCTGAATTTTACACGTGTTGATGAAATTGAAAGAAGACAGGGACGCTGGGATTTCTAAGC
AATGGGTCCGCCTGTGGGTGCCTCGTGGCGTCCTCGGAAACGCACCCATTCTCCCGGTTTAAGAACAGGGTGTCCTTGCACCCCCAGGC
TCCCCTTCCATACATTCTTCCTTGGCTTGCGTGTGGCCTGGCCTCCCTCCTAGCTGTCCTCCTCTCCAGAGCCGCCACTACCCCACCTC
CACAGGTCTCGGAGGACCCTCTTAGCGAAAGAAGCCCCCCTCCCCCTTCCCCCGCTCTTTCCCAGTTTGGACAGAAGCAGTCCGAGCGA
TTTGCATATCTACGAAGGCTGAGGGGGAAGGGNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNCTTTATTGCAAAGCAAAA
AGTGTTTATTAATAATTGGGGGCAGGGCAGGGAGCGGCAGTAGGGGCGCTGGGACAGGCACGAGGGGCCTCGTGGCGGCCGGGAATCAC
TAAATAAGACAGTGTGAGGAATATTTGGGGTGGGGGATTCTTTGTTCAAACAGCGGACCTGGGTACCCCGCACACGCTGCCTGGCCCAG
AGAAAACGCCAGCCTGCAAGGTCGCGTGGCCCCCTAGTCAATGAGACTGACTGTTGTGCGTGCGGTTCTGGATTTCGAGGAACCTGTCT
AGGTCAAACTGGGGGCGGACGGCGACAAAAGCACATTTTCACATGTTCACCAGGAGACTCAGGGCTCCCAAAATATTTTAAAATAATTT
TTAAAGTGACGCATAATGCCCTTGTAGAGGCAAACAGCGCCCGCACCCTGCAAAAGGCGGGCCCCCCGGAGTTTGAGTTGCTGGAGCTC
ACCCCCCAATGTCCACTGAGCTCCTGACCCTCGGCTTGGATCAAGGGTCGTCCGATCGTTATTTTCAGGTCGTTTTTACCCACCTAGTT
ATTTTTTAAATATTTTTAAATATTTTTTGAAAAGATGACGTCTGGGAAATGCAGCGCGGCGGCCTGGGACGCCACCATTGTGTCTCGCG
TGCGCTCGAGCGGCGCCCAGCCCCCGGCGCCCCGCCAGCCCCGCGGTGGGGTTCTCTGGCTTGTGGCTTCCTAATTTCAACAGGGGACC
GGGGTCTCCGTGGCCACCATTGCTTCTACCGGCCCCGCCAGCTGCCCAAAGACTTTCCCTTTCACTTTCAGGGTGGGTGGGCACTGGGA
ACCCTCGGAGCAGGAGCGCATCCCAGGGAGTTGGCCACCGGAATCCCTCTCTGTGCAATCTAGGAAAAGGGAATGGGGTCGTATCGAGC
GACGCCCCCCTCCCACCTTGACCGTCAGGGTCCGGATCCCCGCAGGTCTGTGAGCAGCAGAAGTGCGAAGAGGAGGTCTTCCCGCTGGC
CATGAACTACCTGGACCGCTTCCTGTCCCTGGAGCCCCTGCCGAAGAGCCGCCTGCAGCTGCTGGGCGCCACCTGCATGTTCGTGGCCT
CTAAGATGAAGGAGACCATTCCCTTGACTGCCGAGAAGTTGTGCATCTACACTGACAACTCTATCCGGCCCGAGGAGCTGCTGGTAACC
ACGTAACCCACCACCGACAATCCCTCTTTATGCTGGGTCGTGGGAGCTGCAAATGGCAGCAGATCCAGCCATCCATGGACAGATCTCCT
GCCCCACGAAAGGCACCCGCACCGCACCGCACGACACCGAAATGGCAGTGAGCAGAATGCCAACCCATTCTACCTCTGTGCCCGCCCTC
TTTCCGTACTGCACTCCCCACCATGTCTGTGGTGAAACCACTCTGAAATGTGCGCGGTGTGCGCCCCCTAGCGACGTGCAGGCCACGGC
GCTGCCGGTGGGAGATTCTTTTTGTCCGCAGTCTGTCCCCTTAGTATTTCTACAGGCACATTTTGTTGTTTACCTGTATGGGGTCCCGG
GTTGACCCAACTCTTTAGACCTTCTCCAATCAGCCTGCCTTCCTTTTGACCGTGTTCCTCATTCTCTGCAGCAAATGGAACTGCTTCTG
GTGAACAAGCTCAAGTGGAACCTGGCCGCCATGACTCCCCACGATTTCATCGAACACTTCCTCTCCAAAATGCCAGAGGCGGATGAGAA
CAAGCAGACCATCCGCAAGCATGCACAGACCTTTGTGGCCCTCTGTGCCACAGGTAAGGCTCCACCCCCATTCTTCCCAGGAAGAGCGG
GCTCCTTCCCACTCCCCCTCGGGTTAAGGCCTCTTTCCTTGCTATCTATTCTGAGCCCAGCGTGCACATAGTTTGTTGGAGAGATAGAT
GATACTGGGTGCCACATTCTCTTGGCTAGTATCTTGGAGACCTAACAGAATCACACAGCTTCCCTGTGCCAACCTTTCCACTGCCAGGA
AGTGGTACGGCCACAGCATCCTTTCTGAAGAGGTTTTTGCCTCTTTTCCTAGGCTTCCTGCCTTGCCTCTGTTCTTAAGGAGCTCCAGC
TATAGAGCACACACACACACACCACCTTCTCCCAGGCTGAGCCACCTCCTAGGGGTTCCTACATGGATGGGACAAATCGTCCAGCCTCA
GCCCCACGAGCTACATACAGTACAGCCCCAGAGCACCAACTTAACAGGCCCCACCAGCGCTACTGCACGCCAGTACTTTTTCCCTGAGT
CCTGCCTGGGAGGGTTGGCTGGAAACAGAGAAACTAGAACCTCCCTATGACTCCTCCAAGGGACCCTCTCTGGCCCCAGACCCCAGGTA
GATTAGCCTGACATAAGTCATGGTAGACAGCCATGCAGGAACCCACACACATACACAACCCTCCTACTTCTGAGTGGTTTCAGTACTAC
TTCTCACACCTGCTCCTCACCTCTAACTGAGAACCCTACCCAGGAGCCTTAGCAATATTGAGGGGGTGTCGTCTTCTCCCACCTGTTCA
GGACCCCCTTCCTCTGTGGCCCTGATCAGACCTCAGTTGCTATAGCTGTTTGGCCTGTGATGTCAGAAGGGCCGGTAGAGTATCTCCCC
CCACCCCCAAACCTCCACCTCACCCCCCCTGTCTCAGTGACATCTTCTTATGTCTGACAGCCCCATAGCCAGTGTTTCTAACTCCTCGT
GTGAGAATTTGGAAATTCTAGCTTGCCAACTACCTACTGAGAGTGCTGAGCCCGCCCCAAGGGTACCAAACAAGGGGGTGGCAGATGGG
AGTAGCAAGGCCATCGGTAGGCTGGAAGGATCTAAGGGGTGGGGGTGGCCCTGCTGGCAGTCAGGGAGGGAGCTGGCTGGGGTTCCTCT
CACGACAGCCCAGCCTTTCCAAGCTCCACCCACCCTTTTATGGAGCTGAAAGTGCCTTCAGAGAGGCAGCACAGTTTCCCAGAGATAAC
TTGGGGATGGAGAGGCACAACCTCCCCCAGCATAGTTCTGGCAGTAGTGTGCCCCTCCCCGCACCCCTAGATGCCACCAGACTAATCAG
TAAGCGCTATATCTGGGTACACCTGGACTCAGCTCTTCCAGCTCTCCTTACTCCCTTTGTCTCTGCCCCTGGAATACAGGTGGGGGTAA
AGGACAGCTTCGTCACCTGCGGTACGCCAGTGTTCTGTGGTCTGGAGCATTTGTTTCTCTGTAGGAGCAACCAGCTCCTTTGTCACACG
GGAACCACTTTGGGAAGAGGCCAGTGTGACATCACCGTGAATGGAAAGATGCTAACATCAAGGCCAGGAGATCTGTCGTCTGTGTGACA
GGGGTTCCCCCTATAGGAGATGGGAAGGCATAACTTCCCTTCTGCAAACCGCCCCCCCCCCAACCTGGATTCCTGACTGCAGTTGAACA
ATGTGCTAGCTAGGGTTGCGGTTGTTAACACAGGTCGCCAGACCGCTGTGTGGATGGATACAGACAGTGGGAAACTGAACTTAATGAAG
TATGTAGGTGTCAGACATGCCACTAAGCCAGGGTACACCTATGTCATCAGCAACCAGTGTTAGTAGCTAGGGATCCTTATAGGACATAA
CAGATCTTCCTACTGCCCGTCGGAGGGAGAAAAACAGGCTCTGGAAGGGGCTGGGGACTCCTGGTCCTGCTGGCTGGAGTCTCACTCCC
TGTAAAAGGGCTGTTTGTGATTTGAACTTGACTCCATAGCCACGTTTTCGTTCACTGGACTCACTAGCATGAACTGTCCTTGGTGACAC
CTGGAGGTATTGCTTGTCTGCTGCATCCCTTTAACAGCCAAAGCTGTATGTTCTCTCAGCGTGGTCCCCAGAGCGCCACCTCGCTACAG
CAACTCTCTGCCTTCCTGATCCCATTCCTTCCAGCCTCCTTGATCCCAGGCTCTGGGTGACACACAAGATGATTGATCGGAGCTGACAT
CTGTTCATTCTTGTGTCCTCCTCTGGCCTGGAAGCCCTGATTCGCAGGCCAACTGACCAGCTGTCCCTCTCACTATTTCTTTGTTGGAC
GTGACAGCGAAGCAGTTTTCAGACTCATCTTTCGTGGGTTTATGCGGAGAGGCATTCCATCGGTGTGACATACAACTGCAGGATGCTGA
GTCCAGTGGACCTGAGAGGTGTGGAGTTGGTGACCCCAGATGAAGTCCTAAAATGACACCCCACTACTTCCCAGTTGGGACATGTACAT
ATGAGAGTCCCTAAGACTCCAGGGCCCCTCATGAGGGACCTTGGCCTGGAGATCTGAGGTGGCTATTCCCAGCTCCCTCTGAGTCTATT
CCTACTGGGTTTTCCTGGACTGGGGTCACATGTCTTCCCCAACCTGGGTCAGTAAGCCGGGAAGGCTAACTCCCTTCTGTGGAGTCTAG
CCTCGCCTCTGAGGAACCTGGGCCTGTCATCTCTACAGCTTTCTCCTGTGTCTGAGATGTGTCTCTAGTCCTTCCGGTGCTGCCAGGCT
GACTCTGGAAGACACAGAGATCCCTGCCTCCCACCTCACCCGCGCAAATACTGGGATGCAGATGTGAAAGCACCACACCTTCATAGATT
CCATCCTCTTAGGGCGACATAGCCAACCCCTCCTTGATGTTTGCTGGAACTCCTGGGGATTGAACCCGGTGTGTATGCCAGGCAGCATT
TTAAAGTTTCAGCCAAGGCCTTATTCACAGCTAGCATCAAGGCTCTGGCCTGGACACCGTAATCCTCCGATCTGCTGGCGTCTAGAGAT
GCAGCACTGTGTGCACCTAGTGCCACTTAGGTCTCTCCAAGGGAGCTAGAGTTGATGTGGGCATCCATTGGAAGGGCTTGAAGACCAGC
CCCGAGGCTGTGGGTGGGAGCTGAATCATAGGCATGTCTCTGTCCCTCTCGCTGTAGATGTGAAGTTCATTTCCAACCCACCCTCCATG
GTAGCTGCTGGGAGCGTGGTGGCTGCGATGCAAGGCCTGAACCTGGGCAGCCCCAACAACTTCCTCTCCTGCTACCGCACAACGCACTT
TCTTTCCAGAGTCATCAAGTGTGACCCGGTGAGTAAGTCTCATCAAAGGAGGCTACAGCTGGCAAGGGCCTGGGAACCTTGGTGGGCAG
TGCCAAGGCACTCTGTGACTGGGCTGGACCTGAGAGGTGCCAAATCTGGGTCCCCACGAGCCTCTTCCCACAGTCATGGGTCCCAAAGG
CATAGAGGGCTGTGGTGTGGTGACAGTGCCCCTGCAATGTGTATGGGTAGTGGCATGTGAAGCCCGGTCTGGCCCAGCCAGCATAGGAC
CATATCTCCCTCAGTGCAGACGTTGGGGATTGAATCCATAAAGGACTCTGGTATAGACCTACACATTCTAGAACTTTCTTTGTGTGAGA
GAGGGGTTTGCATCAGGCAGACTGTGCTACGTCTAACTGTACCTTTCTGCCAAGGAGTACAGCTGATCATGCTTGGGGCTCTGGCCGTG
AATGTTTTCTCAAGGGCATCACAAGTTGGCATAGACAGGCAAAAGTCCAGTTCTGGCTTGAACTTCAGTTGGGGGTTCAATGGAGGTCA
GAGCCACTGGCATGTGGAAGGCTTCCAGGTGTGACAGCTGTTCTGCCCTGCCTGGCATCAGCCACAGGGACACCCCGTGCCCCATTCCT
ACATATCTCAATTGTAGAACATGCAGCCCCCTTCCCCTAAGTGTCTGGGGCTCCTGAGAACTGAGAATAGGGTATGTGGGCAGGTTGGA
GCCCACAGGCTTCAGAGAGGGAAGGGTGTCTGTGGGTCCATACTAGGCTACTTGGGCCTCACTTTCTCCATCTGTGAAATGGGGCCATT
GCCCTTCCATCCTCTAGCCATGGTGTTCATCAAACAAAGGGGGAACAATACACAAGTTTTGAATGTGGGGGCTTCCCAGGTGTTCCCCA
GGAATCCTCCTGGCTTCAGGAATATAGACAGTGCCCTAGCTTCAGCCTGCCTAGGCAGGGCTTCAGTTCATGAGTCTTATTCCTGGGCA
AATGCTTTCCAGACCCTACCTGTGCTGGAGCTGTCCTCTCTCAGAGGCCACCCAAGGCAAAAAGTTATGAGAGATAGACGCTCCTTGTG
GGGAAGTTCAGGGTACAGCCTGGGAGGAGGAACCTAGAGAGTAGCAGTACCTTTCTAGTCTCTGGCAGCTCTCACCGGATTTAGGGGTG
GCTTGGGGAGTTTCCTCAAGCCTTGCTCTTAGCAAAGCCTGCCTCGCAGGTCAAGGCCCTGGCCACCGGCCTCTGGCTAAACAAGGACC
CCCTCCATCTCCGCCCGCCTCTCCAGGACTGCCTCCGTGCCTGCCAGGAACAGATTGAAGCCCTTCTGGAGTCAAGCCTGCGCCAGGCC
CAGCAGAACGTCGACCCCAAGGCCACTGAGGAGGAGGGGGAAGTGGAGGAAGAGGCTGGTCTGGCCTGCACGCCCACCGACGTGCGAGA
TGTGGACATCTGAGGGCCACCGGGCAGGCGGGAGCCACCAAGTAGTGGCACCCGCAAAGAGGAAGGAGCCAGCCCGGGTGCTCCTGACG
ACGTCCCCCTTGGGGACATGTTGTTACCAGAAGAGGAAGTTTTGTTCTCTTTGTTGGTTGTTTTTCCTTAATCTTTCTCCTTTCTATCT
GATTTAAGCAAAAGAGAAAAAAATATCTGAAAGCTGTCTTAAAGAGACAGAGAGAGAGAGATAGAATCTGCATCACCCTGAGACTACCG
AGCCAGGGGGTGCTACAAAAATAGAATTCTGTACCCCAGTAATCAACTAGTTTTCTATTAATGTGCTTGTCTGTTCTAAGAGTAGGATT
AACACAGGGGAAGTCTTGAGAAGGAGTTTTGATTCTTTTATATGTTTTAAAAAAAAAAGCTTAAGAAACATTGCTTTAAAAAGGAAGGA
AAAAAAATACAGCAAACCATTGTTAAAGTAGAAGAGTTTTTAGGTTGAGAAATGTACTCTGCTTTGCTGAAAAGCCACAGCTTAGGCCC
TCAGCCTCACTCCCTGGCTTGCTCAGTGCCTACAGCCCTGTTACCTGATACCTGTGCTTTATCCCAGCCCTGGGCAGACCTCTTAACCT
TATAGATGGTCAGTGCGACCTCTAGTGGTCTCATGGCGTGTGGCACAACCCCCCTCCCCAGGGCTCAGCTTAATGTGCCCTCTCCCCCC
AACAACCTGCAGGTTCACAGCGCCACCCACACAGCGGTAGGGATGAAATAGTGACATAATATATTCTATTTTTGTAACCTTCCTATTTT
GTAGCTCTGTTTAGAGAGATGCTGGTTTTTGCCTGAAGCCCCTGCAGCCTGCCCACATCAGGTTAAACCCACAGCTTTTGTGTGTCGTT
TGTTTTGTTGTGTTTTCTTTCTCTATGTTCCAAAACCATTCCATTTCAAACCACTTTTGGTCAGCTAGCTGGAGGCAGTGTTGCTGGTG
TGTGTTGGGCCGAGGGGTTCTAATGGAATGCATGGGGATGTCCACACACGCATTCAGATGGCTGTACAACAGGTTGTAGGGCTGGTAGT
ATGAGGTGCTTGGGAAGTTTTGTTGGGTCAAGAAGAGAGAACTCTGTTCTCGCACCACCGGGATCTGTCCTGCAAAGTTGAAGGGATCC
TTTGGTGCCAGCTGGTGTTTGGAAGTAGGAACCATGATGGCATTACCTGGACAAGGAGATTGGGGACAACTCTTAAGTCTCACACAGGA
GGCTTTTAAACACTAAAATGTCTAATTTATACTTAAGGCTACAGAAGAGTATTTATGGGAAAGGCTGCCCATGACCAGTGTGACTCAAA
GCAATGTGATCTCCCTTGATTCAAACGCACACCTCTGCCCTGCTGGAGAAGGTTTAGGGCCATGTCTGAGAGATTGGTCTTTTATTGGG
CAACGGGGGGGGGGGGGGGGTCCTTAAAAAAAAAAACCACAAAGACAGAGATTTGGTCTGCTTGACTTTCCCAACCCAATTGGCCCCAT
TGGAGAGCCATCCAAACTGAGGAAAATTAGGGGACTCCAAAAGAGTTTGATTCTGGCACATTCTTGCCGCTGCCCCCAAGTTAACAACA
GTAGGTAATTTGCACACCTCTGGCTCTGTGCCTTTCTATTAGGACTTTTTGGCAAAAGGTGGAGAGCGGGAGGCTTAAGAGGGGATGTG
AGGGAAGAGGTGAAGGTGGCACCACATGGGACAGGCCACGGCTCCTCTCATGCCGCTGCTACCGATGACTCCCAGGATCCCAGGCGTTC
AGAACCACATTCTCATTGCTTTGTATCTTTCACGTTGTTTTCGCTGCTATTGGAGGGTCACTTTTGTTTTGTTTTGTTTTGCAATGTCA
GACTGCCATGTTCAACTTTTAATTTCCTCATAGAGTGTATTTACAGATGCCCTTTTTTGTACTTTTTTTTTTTAATTGTGATCTATTTT
GCCTTAATGTGATTACCGCTGTATTCCAAAAAAAAAAAAAAAAAAAAAAAAACAGGTTCCTGTTCACAATACCTCATGTATCATCTAGC
CATGCACGAGCCTGGCAGGCAGGTGGGCGGTCTCCCTCCAGGGATCCTGGGACCCTGATGGCGATCGTCCTGTCATGCTCGGCCCTTCA
TTTGATCTGGGACATACCATCACAGCAGTCAGGGCACCTGGATTGTTCTGTTATCGATATTGTTACTTGTAGCGGCCTGTTGTGCATCC
CACCATGCTGCTGGCCCGGAGGGATTTGCTCTGAGTCTCCGGTGCATCATTTAATCTGTTAGGTTCTAGTGTTCCGTCTTGTTTTGTGT
TAATTACAGCATTGTGCTAATGTAAAGACTCTGCCTTTGCGAAGCCAGCTGCAGTGCTGTAGGCCCCCAAGTTCCCTAGCAAGCTGCCA
AACCAAAACGCCCACCACCACCTCAGCTGACGCATCCCAGCCAGGCAGGACCCTTCAGGGCCGCTCTGTCCATGGTGATCCGCTCACCT
TTTCCCCAAAAGGCCAAAGACTGGTGGTGGCTCCACCGAATCTGCCCTGTGACATGAAAGGCTTTGAGGGACTCTGGCTGGTGGCCAGG
TTGGCTTTTTGTATTTCTGGTTGACACACCATGGCGCTTCCCAGCACAGACATGTGACCAGCATGGTCCAGGAAAAAAAAAAGACAAAA
AATCTAGAAAATAAAATTGGTAAAATCTCAGCTCACTGTTGTCTGTGTTTTCTGGGAACAGGAGTGGGGTGATGCCACAGCCATGTGGG
GTGGCATCTCTGGCCCTGGCACCAACCTGGGATTCCCAAGGGGAAGGGTGTATGCAAGTGACAGCCAAGAGCAAAGTGGGAGCCTCTGG
CGTCAGGCAGGCCACCGACACCCACTGATCAGAAGCCGCTCTCGCTCCATCACTGCCAGCTCACCCTCCTGCTTATTTCCAGATGGAAG
ATCAACAGAACCCGCGTTCACTCCCCCTTGTCTGGCGCAAATGGGTGTTTGCTCTGGCTCAATCACCGTTTCTTAGGTGCTGCCAAGCG
CCCAGACAGCCATGCTTAGTGTTAGGGCCAACACCCAACGTGGCCTTTCACATAGTTCTGCAGACCTGACTCCCCTCGCAACACTCCTG
TGATGTCTTCCCCGTCGCAGTTACTCATGACTAGTATTGTTTCCAAACCCTGTGGGGACAGGTGGCTTTTAGACATCATCTTCTACAAG
GTAGCTTTTGGGTCTCAGCTTGGCTGTTTTGGTGCCTCGTGGGCCCCTGCCCTTCTGGAAAGTCGACGGTCTAGACCACACCTGGTCTG
AGTGGTGCCACCCAATGATGAAAGCAGAAAGAGATGGACGGAGGGAAGCCCACAGTGACCTTGCCAGGCGACCTACTCAATGAGAGTAT
ATGGGAACACCAGAGCCAGAGCTGGCTTATGAAGACACAGTACTCATCTTGTACCAAATTGTCTGATCGCAGGATCCAGAAGGTATTTT
GTCAGCCCACCTCCATATAAGCATCCGACCCCGGTCCACAGTCACATGGCATCCAGGAGAATGGGCATGGGGCCGTCACCATTCCAGGA
AATGGCCAAAGGCCATTGAGCTGGAACAGTGCTTTCCCCCTTGGTTCTTAGAAGCACTGGCGACCGAGCTTATCGTGTCCAGGTACCCT
GACATCAGGAAATCCGCTGGGGTCCACCAGCCACGCAGCCAGTGCAGCAGAGTGGTACTAGGAATGCAGACACAAATAGCAAGGGGCTA
AGCATCCACTCAAAGGCTGCTCCTGCTGCTCCAGCTCCTGCTTTCCACCACTGCCTATCAAAGGTGCTCATGGAAGGGCGCCCAACTGG
AATGGGCAAGCATCACCATCAACTGCACACCGCTCACACATGGTGCCGTCTCTTAACGGTGCAATTGGCTTGTCTCGTCATCCAGTGGT
CTGTGATTCCCAGCTGTGGCTGATAGCCAAGCCTCTTTTCTTGGTTTTACCACTGAAACCCCAGATACCACTGCCCACTCCAGGCTCCA
ACCCTCAAGTTTAACTGGTAACACTGGAGGCTAGATCCTCTGCTGGGTGTAGAGATACTCCTGTTCTAAGGGTGATGGTAAAATAGCTT
TTTGAGGGCCCTCAGAGATAAGGACTCAGGGAGCCAGCACACCCAACTGTCTCCTCTCAGAAGCTCAATGGCTGGTTCTCTCTCTGCCT
CTACCAGCTCCATCGTCCCCTGCCCTGTGTCCTGGCCAGGCTCTCCTTTTGCAGGCGCCCTCCATCCTGCTGAGCCATCCTACCTCTCT
CAGCCAAGCCAAGCTCTTGAACCTGCCTCGCCTCTCTGAGCCTCAGCTCCCTGAACTGGAAAGATTGCCCCTGGCTCCCCATTCTGTGT
TGCCCCACTGCCTGGACCTGCTCCAGCCTTCCTGTTTGTCCTACAGGACTCACAGTGAATGTGGCCCCTGTGGAGCATGTGGAGCATGA
CATTGCACAGGTGACTCTGTCTAGCCTCTCCCCTCCTGACTCCACATCCTACAACCAAGTGTGAGGAAGTTAGCCTGTTGCCCATGGGT
CAGCTCAACTGCTGCACTTTGTCCCAGAAAGCACGCCAACTTCACCAACCCTACAGAGGATCTGGGCTCCTGGGGCCCACAAAATGCCA
GCAGTGGGAGCTGAGGACTCCAAGGGGTGGGACACAGGCAGAAGAGTAGGACTGTGTGTTCTCCCTGCAGTGCCCCAAAGCCCTGGCCA
CAACATCCCAGGAGACAGTCAATGCGGGTTGGGCTGTGTGTTTGGTGGCAAGGCTATGCAGGCCACATAAGGCCAGTGCTGGGAGCTGG
CACTGCCAAGTGTGCTTGCCAAGCAAACCTGTACTTGAGCTGATAAAGCAGTCTGTTGAGTGTGTGGGGTGGGGTGTATAGGTAACATG
AGGCGGGCATGTTGTATACAGGCCCCCACGTCGGCACAGCACAGTGCTGGTGGGATGCACTACCCCTAGTTCTGAGCTGATCTGGCCTC
TCTTACTCAGGGATGTCATCCCACGAATGTATTTGATCAAACAGTTGTGCAAGAGGACAAAGCAAGCACACGANNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNACTAAGTAACTGCT
AGATCCTTGGACTTCCATTCACAGCTGCTACTGAACCATTGTTGGGAATTGGACTGTAGACTGTAAGTCATCAATAAATTCCTTTACTA
TATAGAGCCTACCCATAAGTTCTGTGACTCTAGAGAACCCTGACTAATACAACATGCATACACATAAAATTAAAATGTGCCAGGCAGTG
GTAGCACATGCCTTTAATCCCAACACTTGGGAGGAAGAGGCACGTGGATTTCTGAGTTTGAGGCCAGCCTGGTCTACAGAGTGAGTTCC
AGGACAGCCAGGGCTACACAGAGAAACCCTGTCTCGAAAAACCAAAAAAATAAAAATTAAAAAAATAAGTAGGTAAATTAAATTAAATT
AAAATGTTTAAAAAGAAGAAATCTTGGCTGAATATTCTACAGTTTTTTAAGAGTTTGTCAAACATTTTGATGTTACAATGGCCAATGAT
GACAGTAGCCCCTCACATAAATACATAGTACAGAGGGCAAAAGTGTGCACACCGCCAGTTCAGAAATTGCTGGAAAATTCTGTCCTGAC
CCTAAGACAAGTTTCACTGAACAATTTTTTTATGAAAATGGTCGGGCTGGAGAGATGGTTAAGAGCACTGACTACTCTTCCGAAGGTCC
TGAGTTCAGCTCCCAGCAACCACATGGTGGCTCACAACTATCCATATTCACACCTGACGCCCCCTCCTGGTGTGTCTGAAGATAGCTAC
GGTGTACTTACATATAATAAATAAATAAATCATCAAAAAAAAGAAAAATGGTCAATTTTTAAAATCAGACATTCTAATATAATACAAAC
CATAGCTATACTAATTTGACACCTGTTGAGGCCTGAGAGCACAACACATTAAAGACTTGGCTTGCCATAAGGAACCCGTCATGCCTCTG
TGAGAGCAGAGAGCTGTGTACAAGATGGATGGACGTTGTACTTCTTACAATCCTGGTCTGGGTTGAGGGGGTTTCATTCGGGTGCTGTG
TGTTGGTGCAGATGGAATGACCTCGTCCAGTACAGAGCACAGAACCGAGACTGCATTGAAGTCTTGGGATGCAATATAGGTGAGCGTCT
AGAAGCACCCTTAGATCTGATTATGCATTTGATGTACTTCATTTTTAGACAGGACCTCACTATGTAGCTTTGGGTGGCCTGCCTGAGTA
GACCAAGCTGGCCTCAAATTCCAGAGGATGCATCTGCCTCTGCCTTCTGGGTGCTTGCAATCAAGGTATGCGTGCCACCAGGCCCAGTA
AATTTAATTTTTTCAAATTAATTCTTTTCTCATGTTTAGAAATCTTTTCCTGGTTTTTGTTGTTGTTTGTTTGTTTGTTTGTGACTCCT
ACATTGGGTTTTTGTGACTCCCATACAGAATTGGGATCCACAGTTTTTAAGAAACTGGGCACTAAATTACCCCTGCCTTATGCAAAGCC
CACAAGGACAATCTGGAGAGACCGCTTATGTGAACAGCAGGAGCTGACTCTGTTCAGGGAGCTAAAGGGTAAAACTGTGTATGCTGGAC
CACTCCATTTCTGGCGGTTCCTGGCAAGCCTTAATGTTACTCATCTGGACCTCCCTCTATTTTCACGCTGTCTTGGCTCTTTACGTTGT
AGCTTCTCATGGCCTCCTCCTCAGGGTCCACTACCACCCACCTTAAACAATGCCAGTTCCAGCCTTTCAAGCTGAAATCAAGTGTAAAA
GGCCCAGCCATGCTTGAGCAGCCCTTCCCTGCACAAGTTCAACCACAAGCAAACTTCTGAGTGAACTGGGTGGCTAGGACCCTTCCAGA
GCAGACTTCCCAGCAGGCCTCTGAGCTTGAGAGCAGGTTCCAAAACTCTTGGTTTCAAGGGTCCGCACCCACTGGCAACAGTAAGGAAC
CTTCTGGGAGAATGGGCAGCACTTCAAAGTCTTCAATCCTGCAGACACTTGGTCTTTAGGCACTGATGCAACCTCTCCAATAGAGGACT
AAAGGACCCACCATCCATGAAGCATGGGGACAGGCACTCCAGCCCTGCAACCCAGGAATGGCCAGCGTCTTATGGAAAGTGCCTCTGGC
AAGCTGACCACAGAAAGGCAAGGAGTGACTTCCCTTCTTAGAATATTAACCTGAGGTCACCCTAACTTCTAGGAAATGTGAGCAAAGCC
CTATTCACCCCAGATGGGGCTCCAGTGACAGACCAAAGTATACTTCTGCCACAGCCCAATTTGGTGAACCAATGAGTTTCTAAGTGGTT
AGTTGGTTATAGGAGTGTGGGAGACACCCCAGCTACAGATAATGATCTTGTGGGAGTTAAAAAAAAAGGGTCCCCACTTCTAGTATACT
CCTGAATCCCCTAAGAGCTCTGCTGCTGGGGACAACCAGGTGAAGTAGTGGCTGGAATCCCAGGGTCCTCCCTGCCTTCTCCCTGTCTG
TCACCCCCTTACCATGTGGTTATCTCTGTAACTAGTTGCCACAGCAACCTACCCTACATCATCATTAAGATGGTGGTAGAGTGAACTGA
ACTACAACACAGAGCCACAGGCTGCTGACCTTTGACACAGGTTCCTGGTTGTCAGGGCTCTGGGGTCCCATCCCTCATTTTCTCTGTCA
GCCCTGTGACTTCAGTTTCCTGAGATACACAGCAATTTCTAGCTCTGCTCATAAAGCTGGGAGCCACTAAGGTCAGTGGGGCAATCTTT
CTAGTCAGCACACTTTGCTCATCTCCAGGTGGAACCTCAAAGTGAACATGGGAGTCTCCCTCCCAGGAAGTCTCCCTGCCACCTCCTCA
GCTAACTGACTGCTCCCCTACCTCTGGGCTCCCAGAACCTCGCAGCCATTATCAGAGAGCCCACACGCGGTCTGAAGCATCTCACACCC
CTTCAACACTGCTCCTTCTCTATAAATCCATGCTTTCCTAGACCTGCTAGATTCAAGACATGCCATAGGCTCAAATGAGCAAGAAACTA
GGCTGGAGCAGCAGGCACAACTCTCACCCCACTATCCTTTGTCCCGCCTGGAATTGGCCCTTTACTGTCACCTCCTGCAGTCTCTCCAC
CAGCCCACGTTGCTGACTACAGTCTGGTACATGTAGACTTAGTGGTATGGGGAAGTCAGGGCTTAGCAAACAGAACTGTGGCCCCTTCT
TCCAGTTTCCTCATCTCACTACCAGGCAGAGGAAGAAAAGGTTTGGGGTAAATAGAAAATAAGGAGGGAGTGAGCCAGAGTTTCTGGGT
GTTTGGTCTCAACACAACTATCCTACAAACATCACCATCAGCACCGCTGGGGGATGGAGAGGTGGTTAAGCGTGCTTGATGATGCTCTT
GAAGAGGACCCTGCTTCAGTCCTGACCACCATATGACAGCTCACAAGCACATGGAACTCCAGTTNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNAGGCAGGCCAATTTCTGAGTTCAACGCC
AGCCTGGTCTACAGATTGAGTTCCAGGACAGCCAGGGCTACACAGAGAAACCCTGTCTCGAAAAACCAAAAAACAAAACAAAACAAAAC
AAAACTCCTCACTAACACTAGCTTTATACACAACTTCAATCTGTATTTAGCTCCAGGCTACAATTGCTCCTGCAGTCACACTTAGGGAT
TACATAGTGTCTCTGCAGGCCCAAAAGGCCTTCTGGATCACAGTTTAGACAGACTGCAGGACCATTCTGCCAGGCCCTCTCTCTGTTTA
GTTACTTTTCTGTGTTGACAAAAAAAAAAAAAAAAAAAAAAAAAAAAAGGCAATTTGCGGGATGAAGGAGTTTATTCTGTCCAGTTTAT
AGGTGGCAATCATTATGGTGGGAGGCATGACAGAAGGAGCCTCACGCAGATGCTCATATCGCATCCACGGCCGGTGAACACTGTTGCTC
AGCTCACTTTCTCCTTTCTTTTCAGTCCAGGACCCCACTTCATGGGACACACCGCATTCAAGGTGGGTCTTCCCTTCTCAGTTAAGCCT
CCCTGAGAAGACCCTCCCAGGCACACCCAGAGGTATCTACGAGGTGATTCTAAATTGTCTCAAGGTGACTATCAAGCAGGCACCAAGCT
GAGCAGAAAGACTTGGGGAAACCCAGCCGAGAAACCTCTGCCTCACACCTCCAGCATGTGACAAGTGATTGCTCATAAGGGATGACATC
TAAGCTAGAGTCACAGtAAGCCAGAACAGGCTGGCATGAGTGATAGAAAACAGACCGGTAGTCGGCACACATAGCTCACAGGACATGGT
CTGACAAGGCAGGGCATTGACCAGCAGCTCAGTGATTATCAAGGACACAGGTTCCACTGGTACCTGACTCTGCCACCTTCAGCGACAGC
TTGGCCACAGCTCCCACACTTGTCTATGCCAGTCCAAGTAACACCTTCATGGTACACCATCCAAAAGCAGAGCATTCTTCTGACGACCT
ATGCCCTCCCCTTTCACAGCAGGTGTGACTCAGTGGTGGGAGGAGGCGGCATGCCCACGTGGCAGAATGGTCCTGTAGTCTGTCTAAAC
TGTGATCCAGAAGGCCTTTTGGGGTAACCAAAGCATTCTGGGGACTTGATGGGGACTTTGAGAAAGGGTCAAGTGGGCAGAGGTCAAGG
CTTGTACAGGAACATGGAAAGTTATCAGGCAAGCGGCTGTGGTGATGCTGATGGGTGGGAGATCTGACACCTGATGTACACAAGGTGAG
GGGATAGGAGGTAGAGATGAAGGGAGAAGAGAGGGGAAGAGACAAGAGAGGAACTAGCAACTGATTGATACCTGTAGTGAATGTCACAG
GCACAGTAGGCCCATATCTGTGAACCTACATCTTGACATGCACCATCTTGGCACTCAAGCACTGCTGCAGCTGTCAGTAGAAATGATCC
CACACAGACTACTGCTTTAAAATACACTATTTATTCTTTTTTCTGAATATAATAGGAAAGTAAAATATGGTGATAACAGCAGCTCAGTT
CTCACACCGAAGAGTCCCTGATCCTGGGCACCTGAGAGAGTGGTCCTGAATACCTGAGAGGTAGCCGCCAGGCCCAGGAGGCCAGGTGC
ATGGTGCCCCAAGCCTCAGACTCAAAACATATTTACAAATCCTTCGGACATACAGCTAGGCTCTATTCTGACTAGATTATAACTGAAGA
TAACCCATTTATTTTAACCTGCATTCTCCCAGCTGGCTCGTTACCTGTGCTCAGGTACCATGTGTCCATCTCCTCACATCCTCCCTGGT
AGATCTCCCACCTGGCTCTATCCCAGAATGCTTTCTCTCTCCCAGATGTTCCGCCTATTTCCTGCCTAAGCCATAGGCCATAGGCTTTT
TGCCACAGGTGAGGCATCCATACAACACAAAGATATTCTCTCTACGAAATGCCTGAGAGCACTGTGCCACTCCTGCCAGCTCGTCTTAT
ATCTGCCTGTAAATACCATGCCAGACACACAAGCTGTTGCTAAAGGAAGTACAGTGTCACCAGGCAAGCCCTACACATTCACAGTGGCG
GCCACCAATTAGTCCTGCTTTGGTTTCCAATAGTTCCCCTAATGCCACTTTTTGTTTGTTTGGTTTTGAATTCACTTTGTAGACTAGGC
TGACCTCAAACTGAGAGATCTGCCTACTATCTCTTGAGCACTGGGATTAAAGGCGTGCACCACCAGCACCCAGCTCCATTCCCACTCCT
TAATGTATACAGCTCACCCCACACCTGTTAGGAGCAGCATGAAGGCCCTGCTTCACCTTCCTCCCTTGTCTCGTCACCCTTGTCTTCTC
AGCTGGGCATCCAGAGGCAAACCAAGTCCAGTGGAGCCTGAGTCCTGTGCAGATAGCAGGAATCAGGTCCTCCAGTGGTGTTGGCAATG
ACAGCGCATGTTGGAGGCAGAGGCACCATGTGGTACTTCACGGCATGGACCTGGGGTGGGGGCCAGCTCTGCCACCACGCCCTTAGACT
AGATGGGTCTATTTTATCACTTCTAAGCCATAAGGTATCCTCCACCTCTGGGGCTGCCAGAGGGATGGAGTGAAACAAGGAGGCCCAAG
CATCGCCAGCAGAGGCTCAGCAGCCAGCAAAGATCCTGTCACGGCTTCCCATGACCCATTTCACATCGGCCTGCCTCTGTGACTCCCAC
CCTCAGGGTAACCCTACCCAGGGCCACTCACTCTGGCCAGTGACAAGCCGTCTTTTGAGTGTGTTGCCCCTGTGCAGAAGGCAGGGTGG
TCAGGTAAGCCACCTAGGCAGAGACTCAGGGAAGAGGCCAAGCTGGTCCTCCCACACAACAATGGATTCCTCTCAGGCTTGCCTTTAAT
CACTGTGACACTTAATGTGCAGACATCTGCTCTTCTGTGGGTCCTCAAAACGCAAGTCCAGAGCCTCCTGGAGTCACCTTGAAGTCTGG
CTGCACGTTGAGCAGTGACCACAGCTGAAAGTACCCAAGAGCCCATGCCTTACACCTCCTGCACTTCTGCTCAGCTTTCCCTATCCCAG
CACAAGCCCAAAGGCTATGTCGCCTTAACGTCCCCAACCCACTTTCAGTCATTCTACAAGGGCCAAGGAAACAAGTTCCTCTGGGTCTA
TATCTGGGCACACATTGCCCAACACAACCATCACATGAACCCTAGGGCCCACGCCAACCTGGCCTTCAAAGCCCCCTCAGGCACACCCC
ATAGAC

| | |
|---|---|
| MOUSE SEQUENCE - mRNA | (SEQ ID NO: 14) |

GAGGCCTGTCGGCGCAGTAGCAGAGAGCTACAGACTCCGCGCGCTCCGGAGACCGGCAGTACAGCGCGAGGCAGCGCGCGTCAGCAGCC
GCCACCGGAGCCCAACCGAGACCACAGCCCTCCCCAGACGGCCGCGCCATGGAACACCAGCTCCTGTGCTGCGAAGTGGAGACCATCCG
CCGCGCGTACCCTGACACCAATCTCCTCAACGACCGGGTGCTGCGAGCCATGCTCAAGACGGAGGAGACCTGTGCGCCCTCCGTATCTT
ACTTCAAGTGCGTGCAGAAGGAGATTGTGCCATCCATGCGGAAAATCGTGGCCACCTGGATGCTGGAGGTCTGTGAGGAGCAGAAGTGC
GAAGAGGAGGTCTTCCCGCTGGCCATGAACTACCTGGACCGCTTCCTGTCCCTGGAGCCCCTGAAGAAGAGCCGCCTGCAGCTGCTGGG
GGCCACCTGCATGTTCGTGGCCTCTAAGATGAAGGAGACCATTCCCTTGACTGCCGAGAAGTTGTGCATCTACACTGACAACTCTATCC
GGCCCGAGGAGCTGCTGCAAATGGAACTGCTTCTGGTGAACAAGCTCAAGTGGAACCTGGCCGCCATGACTCCCCACGATTTCATCGAA
CACTTCCTCTCCAAAATGCCAGAGGCGGATGAGAACAAGCAGACCATCCGCAAGCATGCACAGACCTTTGTGGCCCTCTGTGCCACAGA
TGTGAAGTTCATTTCCAACCCACCCTCCATGGTAGCTGCTGGGAGCGTGGTGGCTGCGATGCAAGGCCTGAACCTGGGCAGCCCCAACA
ACTTCCTCTCCTGCTACCGCACAACGCACTTTCTTTCCAGAGTCATCAAGTGTGACCCGGACTGCCTCCGTGCCTGCCAGGAACAGATT
GAAGCCCTTCTGGAGTCAAGCCTGCGCCAGGCCCAGCAGAACGTCGACCCCAAGGCCACTGAGGAGGAGGGGGAAGTGGAGGAAGAGGC
TGGTCTGGCCTGCACGCCCACCGACGTGCGAGATGTGGACATCTGAGGGCCACCGGGCAGGCGGGAGCCACCAAGTAGTGGCACCCGCA
AAGAGGAAGGAGCCAGCCCGGGTGCTCCTGACGACGTCCCCCTTGGGGACATGTTGTTACCAGAAGAGGAAGTTTTGTTCTCTTTGTTG
GTTGTTTTTCCTTAATCTTTCTCCTTTCTATCTGATTTAAGCAAAAGAGAAAAAAATATCTGAAAGCTGTCTTAAAGAGAGAGAGAGAG
AGAGATAGAATCTGCATCACCCTGAGAGTAGGGAGCCAGGGGGTGCTACAAAAATAGAATTCTGTACCCCAGTAATCAACTAGTTTTCT
ATTAATGTGCTTGTCTGTTCTAAGAGTAGGATTAACACAGGGGAAGTCTTGAGAAGGAGTTTTGATTCTTTTATATGTTTTAAAAAAAA
AAGCTTAAGAAACATTGCTTTAAAAAGGAAGGAAAAAAAATACAGCAAACCATTGTTAAAGTAGAAGAGTTTTTAGGTTGAGAAATGTA
CTCTGCTTTGCTCAAAAGCCACAGCTTAGGCCCTCAGCCTCACTCCCTGGCTTGCTCAGTGCCTACAGCCCTGTTACCTCATACCTGTG
CTTTATCCCAGGGGTGGGCAGACCTCTTAACCTTATAGATGGTCAGTGCGACCTCTAGTGGTCTCATGGCGTGTGGCACAACCCCCCTC
CCCAGGGCTCAGCTTAATGTGCCCTCTCCCCCCAACAACCTGCAGGTTCACAGCGCCAGCCACACAGCGGTAGGGATGAAATAGTGACA
TAATATATTCTATTTTTGTAACCTTCCTATTTTCTAGCTCTGTTTAGACAGATGCTGGTTTTTCCCTGAAGGCCCTGCAGCCTGCCCAC
ATCAGGTTAAACCCACAGCTTTGTGTGTCGTTTGTTTTGTTGTGTTTTCTTTCTCTATGTTCCAAAACCATTCCATTTCAAAGCACTTT
TGGTCAGCTAGCTGGAGGCACTCTTGCTGGTGTCTGTTGGGGGGAGGGGTTCTAATGGAATGGATGGGGATGTCCACACACCCATTCAG
ATGGCTGTACAACACCTTGTAGGGCTGGTACTATGAGGTCCTTGGGAAGTTTTGTTGGGTCAAGAAGAGACAACTCTCTTCTCGCACCA
CCCCCATCTGTCCTGCAAAGTTGAAGCCATCCTTTGGTCCCAGCTGGTGTTTGGAAGTACGAACCATCATCGCATTACCTGGACAAGGA
GATTGGGGACAACTCTTAAGTCTCACACAGCACGCTTTTAAACACTAAAATGTCTAATTTATACTTAACGCTACAGAAGAGTATTTATG
GGAAAGGCTGCCCATGACCAGTGTGACTCAAAGCAATGTCATCTCCCTTGATTCAAACGCACACCTCTGCCCTCCTGGAGAACGTTTAG
GGCCATGTCTGAGAGATTGGTCTTTTATTGGGCAACGGGGGGGGGGGGGGGGCGGTCCTTAAAAAAAAAAACCACAAAGACAGAGATTT
GCTCTGCTTGACTTTTCCCAACCCAACCCAATTCCCCCCATTGGAGAGCCATCCAAACTGAGGAAAATTAGGGGACTCCAAAAGACTTT
GATTCTGGCACATTCTTGCCGCTGCCCCCAAGTTAACAACAGTAGGTAATTTCCACACCTCTGGCTCTGTGCCTTTCTATTAGGACTTT
TTGGCAAAAGGTGGAGACCGGGACCCTTAAGAGCGCATGTGAGGGAAGAGGTGAAGGTGCCACCACATGGGACACCCCACCGCTCCTCT
CATGGCGCTGCTACCGATGACTCCCAGGATCCCAGGCGTTCAGAACCAGATTCTCATTGCTTTGTATCTTTCACGTTGTTTTCGCTCCT
ATTGGAGGGTCAGTTTTGTTTTGTTTTGTTTTACAATGTCAGACTGCCATGTTCAAGTTTTAATTTCCTCATAGAGTGTATTTACAGAT
GCCCTTTTTTGTACTTTTTTTTTTTAATTGTGATCTATTTTGGCTTAATGTGATTACCGCTGTATTCCAAAAAAAAAAAAAAAAAAAAA
AAAGAGGTTCCTGTTCACAATACCTCATGTATCATCTAGCCATGCACGAGCCTGGCAGGCAGGTGGGCGGTCTGCCTCCAGGGATCCTG
GGACCCTGATGGCGATCGTCCTGTCATGCTGGGCCCTTCATTTGATCTGGGACATAGCATCACAGCGGTCAGGGCACCTGGATTGTTCT
GTTATCGATATTGTTACTTGTAGCGGCCTGTTGTGCATGCCACCATGCTGCTGGCCCGGAGGGATTTGCTCTGAGTCTCCGGTGCATCA
TTTAATCTGTTAGGTTCTAGTGTTCCGTCTTGTTTTGTGTTAATTACAGCATTGTGCTAATGTAAAGACTCTGCCTTTGCGAACGCAGC
TGCAGTGCTGTAGGCCCCCAAGTTCCCTAGCAAGCTGCCAAACCAAAACGGGCACCACCAGCTCAGCTGAGGCATCCCAGCCAGGCAGG
ACCCTTGAGGGCCGCTGTGTCCATGGTGATGGGGTGAGGTTTTGGCCAAAAGGCCATAGACTGGTCGTGGGTCCACGGAATCTGCCCTG
TGACATGAAAGGCTTTGAGGACTCTGGCTGGTGGCCAGGTTGGCTTTTTGTATTTCTGGTTGACACACCATGGCGCTTCCCAGCACAGA
CATGTGACCAGCATGGTCCAGGAAAAAAAAAAGACAAAAAATCTAGAAAATAAAATTGGTAAAATCTCAAAAAAAAAAAAAAAAAAAA

| | |
|---|---|
| MOUSE SEQUENCE - CODING | (SEQ ID NO: 15) |

ATGGAACACCAGCTCCTGTGCTGCGAAGTGGAGACCATCCGCCGCGCGTACCCTGACACCAATCTCCTCAACGACCGGGTGCTGCGAGC
CATGCTCAAGACGGAGGAGACCTGTGCGCCCTCCGTATCTTACTTCAAGTGCGTGCAGAAGGAGATTGTGCCATCCATGCGGAAAATCG
TGGCCACCTGGATGCTGGAGGTCTGTGAGGAGCAGAAGTGCGAAGAGGAGGTCTTCCCGCTGGCCATGAACTACCTGGACCGCTTCCTG
TCCCTGGAGCCCCTGAAGAAGAGCCGCCTGCAGCTGCTGGGGGCCACCTGCATGTTCGTGGCCTCTAAGATGAAGGAGACCATTCCCTT
GACTGCCGAGAAGTTGTGCATCTACACTGACAACTCTATCCGGCCCGAGGAGCTGCTGCAAATGGAACTGCTTCTGGTGAACAAGCTCA
AGTGGAACCTGGCCGCCATGACTCCCCACGATTTCATCGAACACTTCCTCTCCAAAATGCCAGAGGCGGATGAGAACAAGCAGACCATC
CGCAAGCATGCACAGACCTTTGTGGCCCTCTGTGCCACAGATGTGAAGTTCATTTCCAACCCACCCTCCATGGTAGCTGCTGGGAGCGT
GGTGGCTGCGATGCAAGGCCTGAACCTGGGCAGCCCCAACAACTTCCTCTCCTGCTACCGCACAACGCACTTTCTTTCCAGAGTCATCA
AGTGTGACCCGGACTGCCTCCGTGCCTGCCAGGAACAGATTGAAGCCCTTCTGGAGTCAAGCCTGCGCCAGGCCCAGCAGAACGTCGAC
CCCAAGGCCACTGAGGAGGAGGGGGAAGTGGAGGAAGAGGCTGGTCTGGCCTGCACGCCCACCGACGTGCGAGATGTGGACATCTGA

| | |
|---|---|
| HUMAN SEQUENCE - GENOMIC | (SEQ ID NO: 16) |

CATGCCTGTAATCCCAGCACTTTGGGAGGCCAAGGCGGGCAGATCACCTGAGGTCAGGAGTTCGAGACCAGCCTGGCCAACATGGTGAA
ACCCGTCTCTACTAAAAATACAAAAAAAAAAAAGAAAAAAAAGAAAGAAAAGAAAAATAGCTGGGCATGGTGGCGCATGCCTCTAATCC
CAACTACTTGGGAGGCTGAGGCAGGAGAATCGTTTGAACCCAGGAGGTGGAGTTTGCAGTGAGCTGAGACCGCGCCACTGCACTCCAGC
CTGGGAGACAGAGCAAGCCTCTGTCTCAACAAAAAAAAAAAAAAAAAAAAAAAAAAAAGGGGAAAAATCACTCCAATCATCCCAAGTCT
CAGCTCCTACTCTTTTGCTATATGTCCTTCAAACCAGCTATGTATTTATTTTGCTGAATTTATGCTGGTTTTATCTTTTGTATCTTGCT
TTTTTTCATTTAATGTTTTTCCATCCAGTGAACACACCCTAATTTGCCATTCCCCTCTTCTTGAACATGAGATTGTCTCTAGGGTATCT
CTTGCAAATAGGATAATTGTCAATGATGTAACGGGTCTCTATGCAACTTGCAAAAGTGATCAGTCTTGGTGTCCTCCTCATCCTAGTGT
TTGGTTCCAAGCAACAGAAACCAATGCTGGCAGATTTCAGCAGAAAAGGAAAATCAGATGCAGAAGATAGGCACGAACAAGGGAGGCAA
GACAGCACGAATCACAATGGAAGCATGCTGCAGATCTTCTCTGATGAGAAGCCAGGGCTACCCCTACCCATCCCAACACCAGACCCTCC
AGTAAAACTGCAGCGGCCCACACCACCTTGTCACCAGAGCCCACAGCTCTATGATGGGCAGCACAGATACTGCTGCTCATAAACTTGAA
TGCCTCACCCAGTTCTACACTAGAAAACATTTCCTACCTTTTCTGATTCTTCCCACCACCAGCTCCCAATTCAAAATCTTGATCCAAAA
AGATGAAATGTATAAATAATACTATTTATAATGACATCAAAATATCAAGTACTTAAGAAGAAATCTAATAAAAGATGCACAACACCCAA
CACACACACACACACTCACACACACACACCTGCAATTAAATTAAGGACAATCTAAATAAACAGAGGTCTATATCATGCCCATGAATTGA
ATGACTCTGTTAGTAAAAGTGTCAATTCCCTCAAAATTGATCTATAGATCCAATACAATCCCAATCAAAATTCCAGCATTGTGTGTGTG
CACATGTGTAAGTTGATTCTGAAATGTTTATGGAGCCCCAAGGGCCAGGAGTAGCCAGGGCAACCATAAAAGAGAAGTGGGTTGGAGGA
TTTAGCCCGCCAGATATCAAGAATTCGTAGCGTATAGCTTCCATGATGCTTCTGGGGTAAGGACTGAAAAAGAGACCAGTGGAACGGGA
TAGAAAGTCCAGAAACAAGAGATTTGTGTTAAGTGAGCAGATTTTAGCTGCTCTGTTCACAAAAAAAAGTAACTATGTGAGATGACAGG
TATGTTACACTAATTCACTATGGTAACCATTTTACTATGTATATGTATCTTATAACATCATGCTGTCAAGTTTATTTCAAAAATAAATT
TTACATAAAATCTTAGCAGTGTTATTCATAATAGACAAAAAGTGGGAAAATTCTATTTTTTTAAAGTACAGAAACTTTATTCCTCAAAG
GGGAAATAGGTTAATAATAATAAGAAGAAGAATTAAGGCCAGGTGTGGTGGCTCATGCCTATAATCCCAGCACTTTGGGAGGCCAAGGC
CAGAGGATTACTTGAGCCCAGGAGTTTGAGACCAGCCTGGGCAACATAGTGAGACCCCATGTTTACAAAAATTAAAAAATTAGCTGGGT
GACGTGACAAGTGCCTATAGTCCCAGCTACTTGGGAGGCGAAGGCAGGAGGATTGCTTGAGACCAGGAGGTCAAGGCTACAGTAAGGTA
TGATCGTGCCACTGCACTCCAGCATGGGCAACAGAGCAAGACCCTGTATCTAGGGGGAAAAAAATGATAAGCCTTGTGAAAGAGAAATG
AAAACAGCATCCACACATAAACTTGTACCTCTGTGTTCATCGGCAGCATTGTCCACAGTAACCAAAGGTAGAAACCATCCTAAGGCCCA
TTGACTGATGAATGGATAAGCAAAATGTGGTCTATCAATACGATTGATAGTATTCAACCATAAAGAGGAATGAACCATGGACACATGCC
ACATGTGGGCCTTGAAAACGTGATGCTAAGTGGAAAAAGCCAGATGCAAGAGGCTGCATAGCATATGATTCCATCTGTTGGAAATGTCC
AGAAAAGGCAGATCCACAGAGAAAGATTAGTGGTTGCCAGGGGCTGGGGAAAAGGGGGAAGAAAAGGGTGAATAACTACAAATGGGTAC
AGGGTTTCTTTGGCGGATGATGAAAATGTTCTCAGATGGTGATGGTGGCTGTCCAACATAACAAAATCCATCGAACTATATACTCTAAA
CGAGTGAACTTTACGGTATGTAAGTTATTTTTCAATAAAGTGGTTTCAAAAAAATAAAAAGGTGAACCCTACCATTTCTAGGTGCTTTG
GAGTTTACCGTTTTTCTCCTTGCATTCTGGAGTCAGAGTGAGAAGGCATCTCAGCTCTACCACAGCTGAATGATGTGTTGGGCTTCGGT
TTCCTCATCTGTAAAATGGGGATAAAGGGCCTCTCCTTGGAGCTGCCATGAAGATTGAATTAGATGCCCTCTGTGAAGATGCAGGGCAG
GTGGAGAGCACAGTAAGCGTCTCGGGGGGCCACGCCATCACCGCCAGCAGGGCTCACCAGTATCTTTGCCCAGAGAATAGCCGGGGTCT
AGGGAGCAGCCAGGGTCTAGGGAGAAGGGCTGCTTCCCTGTGGAGGCTCCTAACTTTCCCATCTCCTCCCTGTTTCTCCCCTGCCCACC
CCTTACTCCTGGAAGTGTGACTCGCTGCCTCCTCCCGTGACTCCCCTACATTGCATCTCCCCAGTTACTGTCGTTATCTCTCATCTCTT
CTTTCCAAACAGCCTCTGTCTAGCACCTTGGAGCTCGCATGGGAATTCATGGGTGGGTGGATTCCAAGCTTGGCTTCATCAGATGACAA
CTAAAACCCACAGGCCTGGGGGGGCAACGGAGCTCCAGCATCTGTCTCCCTTTGGAGAAGCCCAGGTTCACCTTCCCAGCAAGCTGCTC
CCGCCCCACGCCCCTGGCCCTCAATAACCTTCAGTGGCTCCCTTCTTGCAGCTATAATTCCCCTCTTGGGATGCTACAAAAGGGATTCC
ACACATTTTTTTTTCAATAACTCAAAAGGGATGGAGTAAACGATATATGTGCCCAGGACAAGGCAGCTAAGGGAAGCCCTCACCCTTCC
TTTGCCTTTCCCTGGAAGTATACCAACCCCCAGCCCGGACGCCCAGCAGAGAACCAATGCTCAGAGCCTGGGGGCCCAAGGAATGCTTT
CCAGCCTGGCGGTCCCAGGAGCCCCCTCTCCCTTCCCTATAGCAAGATACTAAGAGCAGACCCTGGCCTGGCACCCCCTGTCGACGCCC
TGAGCTCACATCGCCTTCGCACCTTCTTGACAGTCGTGTCTTTTTGTTTCCAGCAGTGACACTCACTGACCCACACCTTTGGCCACCCT
CCCTTGTGACTGCCCCCTCCCGCTTGAGCACAATGGCTCCTATTACACAATGGGTGCCACGAACCTGGCCACTCCATCCAGAGACAGCG
CAGAGCCCTGGGCTACCGACCCTCTGGGGCCAGCAGAAACCCTCCCTATGGCTCTGCCTCTCATCCTGGCATTGACACTTGTGCCCTTA
ATTAAAGTGCTTCTTTTTTAAATCAACATTTCTACTCCTTTTGCAGAGTAAGAGAAAGAAGAGTTAGGGGAGAAGAAAGAAGGAAGGAT
TCATGGGGTTATTTTTTTTTTTTCATGTAAAAGTTTGTTCCGTGATTTAAATCTCATGTCCACAAACAGCTCAGGAGAAGTCAGCATAG
ACAAGCAACCTGACACATTATAAAGTCATTTGTAAAATCCTCTTTCTGCATATAAACTGAAATGTGTAATTAATTCCACATACTGCGTG
TGGCACATGGCCTTCATCCTGCATAAGAACAAATACATGAATGAATAAATCGAGCAGGCCTTGACACTCACCACGCCCTGTGCTAAATG
CTTTCTACATATTACCTTAATTACTCCTCACCGTGGCCTTGCGAAGAAAACCATCACAGCCCGTTTTACAGATGGGGACAGAGATTTGT
CCAAGGCCACACAACTAGTGGCACATGTAGGTCTGTATGTGACATTCTTGTTCAGTTTCATAAATGTGGCTCCCACCTGCCTGAAATTT
TGAGGCAAAAAAAAGTGGTTTGGCTTTGCATGAATTCTTCAAAAAGTAGATTTTTATCTTTACCAGCTTCTAAGCAAGCTGCATTTATT
CTGACAGTGTCCATGAAATGGCTACAGACCATCACCGTTGCCAATGTTGTGAACCTGAACAAGGCACCTCGTTTCTCTGTGCCTCGGTT
TCCTTACCATTAAACGCATTTCCCTGAGATTAACTGTCTCAAACTATAAGTTGGCTGGAGGAAACACCTCATAGAAGTCAGAAGATGAG
GATGCGTGGTCAGTGAAGGCTTTTTCTCCAAACCCTACAGGAGATGGACCTCAAAGCAAATGGCCATGCTTCCCTTAGACTGGGTGAAC
TGGTGCTGCTCAGGGCTAGAACTGTCTCTAGAAGCATGGGGACCAATGCAGCCCCCTCCACCCTAGGGCACATAACCTGTCTCTCTCCC
ATAGGATAGCAGAGACAGCTCTCTAAGTCAGGATTCAGCGAGATGGTCCAGGAGGCGCCTAAGGATTCCTCTGACTTATCCAAGCCTGG
GGAGGCAACACAACCTACCCTGTGTCTCAGGGGGCCCCAATCATGCCCTGAACCTCATGAGCCCAGGCAGCCATCCTGTTGGTTAAGGT
CGGCCTCAGGGTCCAGGGATGCACTAGATTCTTTATCTTAAAGTGACCCTCAAGAAAAAAAAAATAAAATGTAGCTATAGGCAGTTTCT
GGACGTTTTCCTATTTGTTAAGCACCTTCTTGCTCCGAGCTTTCCAGCCACAGTCCACTGGACCCCATCGGAAGGCTGCAGCTGCTATT
TGGTGCTTTACCACCGCTGAGAAGGTGGAATGGTCACGGCCCGGTTCTGTCACTTTCTTCAGCCGGACAGTCGCCTTATTACCGATTCC
AGTAGGGCCGAGCACACTCCTTCCTGCCCGTCTTTACAGATGAACATGCTATCGCTCCAGCAGTACAACCCGCCTTATTACGTAAAAAA
GCAGCCCCTATCTACCCCCAGCGAAGGAGTATGTTCGGCACCACAGCTGGACTGGTGCTCGAGTTAAGCGTCCTGGGACGTCCGCATGC
GCTCCGGAACCGTAATGCGCGCTTTTTCTAAGCCTTACGGTAAACGCGCACGCAGGGCAACCACGTGGCGGTGGAACCGAGGCCCGGCG
GGAATGCCCCCAATGCGCGGCGCGGCCTCGCGCGGTTCCCGAGCCACGGCCCAGGGTCCGGCGGCCCGCGCTCTCGCCTCCTCCCCTCA
CCTCTCCCAGCCGCACCCCGGCCCTGGCCCTGCCACCCACAACTCGCTGGGCAAGTCGTGCCCCGCGTGAACACACAGAAGGGGCTTGG
GGACCGAGCGCGGCCCATCAGTCCCTCAGACCCTGAGGACCCAGAATTCCCTAAGCGGTCCGAATCCGAGTCCTGCCCCCAGCCCTTAA
GGCACGGGCTCCAGGGACCCCAGGGGAAGGGCGCGGGGCATTAGGTACGCAACCCGTTTCCCCGCACCTGGAAAAAAACTCCCTTTCCC
TCCCCTCCCCTGCTTGTTGAGTGTGCGGATAACCAGAACTCTAACCCGCCCCGTAATAACGACCCCGCTGTCCCTCCACCCACCCCCAA
GTGCCAAAGCGAGGGATGGAAGCGCTTTCAAGCGTTCCAAGGGCATTGAGGAGCGAGCTGGAGAGGCGCGGGGATGCGGGGTCCTCCCC
GCAGTCTTCCGGAAAGGGCGGGCGACCCCCCGGCAAGTTCCGGAGTGGGGCATGCCGTGGGAGCCCACGAGGGCCTCAGCCCGGATCCT
CCGCCGGAAAACCGGCTCCCGCGAGCCGCCGCCGCAGGTTTCCTAGGCCCCGCGAGTCCCGCAGCGAAGCCCTGCGTCTCCGTCCGACG
CGGGGGTCTGCTCAGCCTCGGGTCGGCCGCGGCCAGGCCTGACTGCGGGGGAGAGGGCCGAACCTGACCTCCGAGGTCACCCCCAGCCA
GCTTTCTCTCCTGTGGTCGGAAGTGGTTTTCTTCTCGATCTGGGCGCCTACTCCCCACCACTTGGTCTGAGAGGGGCTGGGGCCGGAAG
GCCAGGGAATCTCTGGTGGATTTGGGGGTTCATATTGCTCAGGGTACCACCCGATGCGTTTTGAGGGGCGGGAGTCGAGGAATTAGAAT
CGCCTTTAACCCTCAAGACTTCCGCCTTCAGCCTCGGGATCCCAGATGCGTCGTTGGAGCCAGGGCCGCCCCCCTACCTGTTGGGTTTG
CGTTTTAACTCCAGCGCACACCTTGCCGGCAGCCCTCGGAGCTAGCGGAGGGGTCTCGTTTCCCCGCAGCCCGCCGGACAGACGACTGG
GGCACCCCAGGGGCGGTGGCAGGGTGGTCTGTGTGTGGCTGAAACTAATTGATCTGGACCCGAAACGCACGTCTGCGGTTGGGGCGATG
GGGGGGGCGGTGCGGCTGTCCATCTGCCGAGCGTGTGGCTGTCTCGGGTGGGCACTGGCGCCGGAGTTCGCCCCCGCCCACCTCGCAGT
TTTGGGGCGCCTGGGATCGGCGCTACGTAAGCGAAGCAGAGCTGCCATAGCACGTGGGCCGCCACGCGCACCCCAAAAGCAAGCAGTGT
GGGGGGAAGGGGAGCTCGAGCGCCTTCGGAGCCCAGGGGCCGGCTTTCGGAAGCGTTTTCCCGCGCGACTTAAGGGCTTAACAATGGAA
AACTCGCGGAGCCTGAGCCAAGTCCTTTCAAGTCGCCGCCAGGTATGCGGCTGCAGGTGACCCCACCTGGGTGCGCCCGCCCGCCAGCC
GCCCTGGTGGAAAAGCGGGTGCGGGAGGTCGCTGGCGAAAGGTCGGGACTGGTCCCTGCACCACCCGCCCCCAACCCAAGCCCCGAGCC
CCGCGGCGCGCACCCGCGCTGAGTCCCGGGGTCTGCGTCGCGGCGCGCCGGTTCCTGAATGAACCCGCTCCCTTCCCCCGCCTGAATGA
AGGTTCCCACACCCAGGGACGGTGGCGAACACGCGCCTGCAGCGCAATTCGCTTTCTCCTCACCGACCATCCGCCCAGGCCGCGGTCAC
CGGGGCGGCCCCCAGGGGGCGAGGAAAGCGTGAAGGTGATTTCAGTTAATTTTGGATTTTCTTTCAAACAACGTGGTTACCCTCCCGAC
TGGGCCACTTGCCCTTTGTCTCCAAATGGTCACCAAGAAATAAGAACAGAGCACTTTAAATGAGCCCAGAATCCGCAGTTCCTGCTTCG
TGGTGGGTTTTAAGAAGACAGTGTAAAGTAAAACTGCAACCGAAAAGTTTTTTAAAGTTGCTTTTCTCTTTGGAAAAAATAAAATCAAA
ATGCTTTCTCTGCGCTTCTTGAAGCAATGACCCTCAAAAGCCCAGAGGTATTGGCCCCCTCGGGGGACCCGGGGGCCGCCAAGCAGGGT
TCCCCCAGGTGGGGGCTGGGCAGCTGGCGCTCCCCGCCGGGCCCCAAATTCCAGCGCCGGGCCCGCAAATTCCAGCGCCTCCCCCGCGG
GTTCCTGGACGGCTCTTTACGCTCGCTAACCGGGCTTGCAATTTTGCGCTCGTCCCTGAGCCGGGAAATCAACGAAGTTCCTAGTCGAG
ATCTGCCCGGTCCGCCTAGTAACAGCCCCGCGCCCCCATTGGCTCATGCTAATTCCAGTTTCCTCTGTCTTGCGCCCGGGATGGGGGGG
TGAAGCTCCCTCCTGGACCCAGACCCGGTTGTGCCGGAGTGGGCGAGCCTCTTTATGCCCTGCTGCCCCTAGCCGACTTCGGCCCGCTT
CGCGCCTCGGGCTGGGCCAGCCCGCACGCGGGGCTCGGGGCCCCTCGCCCCACGGGATGGGAGAGGCCGGGTGATAGCTCCGGGCCCCA
TAAATCATCCAGGCGGCCGCCGGGTCGGGATTTTATGAATCAAAAAGCAGCTGGGCCGCCCTTGTGCGCGGGCTGATGCTCTGAGGCTT
GGCTATCCGGGGGCCAACGCGATTGTGGGTGCTCGGGGAGTGGGGGGGGGCACCACCGTAGGTGCTCCCTGCTGGGGCAACCCATCGCT
CCCCATGCCCAATCCGGGGGTAATTACCCCCCCAGGACCCGGAATATTAGTAATCCTAATTCCCGGCGGGGGAGCGCCCGCCGCAGGAA
TTCACCCTGAAAGGTGGGGGTGGGGGGGGTCGCATCTTGCTGTGAGCACCCTGGCGAAGGGGAGAGGGCTTTTTCTATCAGTTTTCTTT
GAGCTTTTACTGTTAAGAGGGTACGGTGGTTTGATGACACTGAACTATATTCAAAAGGAAGTAAATGAACAGTTTTCTTAATTTGGGGC
AGGTACTGTAAAAATAAAAACAAAAGTTAAGACAGTAAAATGTCCTTTTATTTTTTAATGCACCAAAGAGACAGAACCTGTAATTTTAA
AAACTCTGTATTTTAATTTACATCTGCTTAAGTTTGCGATAATATTGGGCACCCTCTCATGTAACCACGAACACCTATCGATTTTGCTA
AAAATCAGATCAGTACACTCGTTTGTTTAATTGATAATTGTTCTGAATTATGCCCGCTCCTGCCAGCCCCCTCACGCTCACGAATTCAG
TCCCAGGGCAAATTCTAAAGGTCAACGGACGTCTACACCCCCAACAAAACCAATTAGGAACCTTCGGTGGTCTTCTCCCACGCAGAGGG
GACTAATATTTCCAGCAATTTAATTTCTTTTTTAATTAAAAAAAATGAGTCAGAATGGACATCACTGTTTCTCAGCTTTCCATTCAGAG
GTGTGTTTCTCCCGGTTAAATTCCCGGCACGGGAAGCGAGGGGGTGCAGTTGGGGACCCCCGCAAGCACCCACTGGTCAAGGTAGGAAG
CCAGCCCGAAGAGTCTCCAGGCTAGAACCACAAGATGAACGAAATGCTGGCCACCATCTTGGGCTGCTGCTGGAATTTTCGGGCATTTA
TTTTATTTTATTTTTTGAGCGAGCGCATGCTAAGCTGAAATCCCTTTAACTTTTACGCTTACCCCCTTGGGCATTTGCAACGACGCCCC
TGTGCGCCGGAATGAAACTTCCACAGGGGTTGTGTCCCCGGTCCTCCCCGTCCTTGCATGCTAAATTAGTTCTTGCAATTTACACGTGT
TAATGAAAATGAAAGAACATCCAGTCGCTGAGATTCTTTGGCCCTCTGTCCGCCCGTGGGTGCCCTCGTGGCGTTCTTCCAAATGCGCC
CATTCTGCCGGCTTGGATATGGGGTGTCGCCGCGCCCCAGTCACCCCTTCTCCTGCTCTCCCCAGGCTCCGTGTCCCCTGCCGGCCTTC
CTAGTTGTCCCCTACTGCAGAGCCACCTCCACCTCACCCCCTAAATCCCGGGGGACCCACTCGAGGCGGACGCGGCCCCCTGCACCCCT
CTTCCCTCCCCCGGACAAACGCTGCAGCGGGGCGATTTGCATTTCTATCAAAACCGGACTACAGGGGCAACTCCGCCCCAGCGCAGGCG
CGGCGCCTCAGGGATGGCTTTTGGGCTCTGCCCCTCGCTCCTCCCGGCGTTTGCCGCCCGCGCCCCCTCCCCCTGCGCCCGCCCCCGCC
CCCCTCCCGCTCCCATTCTCTGCCGCGCTTTGATCTTTGCTTAACAACACTAACGTCACACCGACTACACGGGAGTTTTGTTGAAGTTG
CAAAGTCCTGCACCCTCCAGACCCCTGTCGGCGCAGTAGCAGCGAGCAGCAGAGTCCGCACGCTCCGGCGAGGGGCAGAAGAGCGCGAG
GGAGCGCGGGGCAGCAGAAGCGAGAGCCGAGCGCGGACCCAGCCAGGACCCACACCCCTCCCCAGCTGCCCAGGAACAGCCCCAGCCAT
GGAACACCAGCTCCTGTGCTGCGAAGTGCAAACCATCCGCCGCGCGTACCCCGATGCCAACCTCCTCAACCACCGCGTGCTGCCGGCCA
TGCTGAAGGCGGAGGAGACCTGCGCGCCCTCGGTGTCCTACTTCAAATGTGTCCAGAAGGAGGTCCTCCCGTCCATGCCGAAGATCGTC
GCCACCTGCATGCTGGAGGTGCGGGGCTTCGGGCGGCTCTCTTAAGACTTCCCTGCAACTTGTTGCCCAGACCCACGTTTCTTTGCTAC
TCACCCCCCTCCCTTCTCTCCCCCTAGAACTTTGAAGTTTGCCGTGGTGTTTCTAGGGATCCGTATTTTCAAATAAAAAATTGCGGGTA
TTTTCTGAAGGAGGAAGGGGTGGGGGTGGGGGTGCTAGAAGTAGCGTTTCGTGGGACGGGAGAAGGGGGTCCGGCAGGCGTGCCTTCGG
GAGAAGCCAGTGCCAGGGGCACCCCAATCCGCCCGAGGGTGCGGGCTGGCAGGCTGGCTCCGCTTTGTGTCCCCCGCCTGCGCCCCAGC
CCGGCTGCGCCTCAGCGGCCCGGAGCCGCCAACTCCGGGGGGAGGGGGCATAGATTTGATTTTTAAATTAATATCCATGGACACGTATG
CAAGGGCCGCTCGTGCCAGTATTATGCGCCATCTTTGCTCTTTTATTGCAAAGCAAAAGTGTTTATTAATAATTGGGGGCAGGGTGGGG
GCGGGGAGCCGCCCCCCGGCCCTGGGGCCGCAGCTAAGGGCCGCGCGGCTGCCGGGAGCCCGCGGGAGGGCCGCAGGGACGCGGCATGG
GTAGTTTTGGGGGGACCCCGCTAGGGAAGGGGGGGCCTTTGTTCAAGCACCGAGTCCCGGGGCGCCCCGAACGGGCAGCCTGCGCCGGA
GAGCACCCCGAGCTGCAAGGTCGCGTGGCCCCCAAGACGCCAGGGCTTGATCCCCGTCTGCAGGCATATCGGCTTGCAGGACCTTCTCC
GAGCGAGCCGGGGGCCTGGGAGCACATTTTCAGACCTTCCGTGGGCGCCTGAGCCGCCCGCAAGTATTTTAAAATAATTTTTGAAAGTG
CGGCGTGGTGCCCTTGCGACAGGGAAACGCCGCCCGCGCCCAGGGGGAAGGGGCGCCCCCGGAGTTTGAATTCCTGGGGCTCCCCCCGG
AGCCTGTAACGAACTCCCAACCCCCGGCCTGGGTAAAGGGTCGCCCGAGGGTCATTTTCAGGGTTTTTTTATGCACTTAGTTATTTTTT
TAATATTTTTAAATATTTTTTGAAAAGATGACGTCTGGGCAAATGCGGCGCCGCGGCCTGGGACGCCACCTTTGTGTCTCGCAGGCGCG
GCGCCCAACCCCGCGGCCCGTTCCGCGGCCCCGCACCCCAGTTGGTGTCGACCCCCAGTCAGACGGACCACGGAGCTCCAGGGCGGGCC
AGCGTCCCGGGGGCCGGCAGCCCGCGCCGCCGCGCACGCCGCCCAGCTGTGCCCGCTCCCGCCCCCACCGTGCCACCCTCGCGGGGACT
TTCCCTTTCAGTTTCGGGGAGGGTGGGTACTGGGGACGCGCGGGGGACGGGGCGCATCACGCGAAGCTCCTGCCGCCCCCAGCCCCGAC
CCCTCGGCGCCCTCCAGACCTGGCGGCCCTGCCAAGCGCGATGGGGGGTGCGGGGGCGTGCGGGGGGGCGGCGCGACCTGCCGGCGGCG
GTCACGGGCCCCGTGCCTCCGTAGGTCTGCGAGGAACAGAAGTGCGACGACGAGGTCTTCCCGCTGGCCATGAACTACCTGGACCGCTT
CCTGTCGCTGGAGCCCGTGAAAAACAGCCGCCTGCAGCTGCTGGGGGCCACTTGCATGTTCGTGGCCTCTAAGATGAAGGAGACCATCC
CCCTGACGGCCGAGAAGCTGTGCATCTACACCGACAACTCCATCCGGCCCCAGCAGCTGCTGGTAACCACTGGACCCCGCCGCCCCCCG
CCCCCCGCCAGCCGCACGCAGGACCACGGGGCCGGGGAAGGTGCAGGCGGTGGCGGCCGGCCCCCCTCTGACATATCTGCTCCTCCGAG
GGAGGGCGGCCCCGCCGCCGGGCGTCCCTGTCCCGGGAGCGGCCGGGATCCTAGCCGCCCTCGTCCCGCCGCCCTGTGTGCGCTTGCCT
GCGACTCCCACCCCGTTCGCGCCCCGCGGTGTGGCCGAAAAGTGGGCGGCGCGCGCCCTCCAGCGGCTGCACGAGGAGCGCCGCGCTCG
GCGCTGAGCCTCCAGTTCCAGGTGGTGCGAGGTCTTTTTGTTTCCACTTGCAGAGTCTTTTCACGCGCCGGGCGCCTTTTCTGTTTTGA
TCTGGGATTGCGTGTTGCCCCAGCTCCCTTGAGTCCCCAGCATTCGCCAGCCCTCCCCTCCAACATCCAGGACCGCACGAGACCCAGGG
GCCAGTGCTCTGAGCCGGAGGTGCGGCGTGGCCCGGCCCCCGTGCTGCCCGCTTCCCCGCGCCCCCGGGCTGGCCCGCACCTCCCCTGA
TGGCCGCTCACCCTGTGTTCGCAGCAAATGGAGCTGCTCCTGGTGAACAAGCTCAAGTGGAACCTGGCCGCAATGACCCCGCACGATTT
CATTGAACACTTCCTCTCCAAAATGCCAGAGGCGGACGAGAACAAACAGATCATCCGCAAACACGCGCAGACCTTCGTTGCCCTCTGTG
CCACAGCTAGGGCAGGCCCGGCAGCCCCCGGCCTCCCCTTGAGAGCCGGCTCCTTACCTGACCCTGGCCGGCTTCTTGCTCTCCACCTG
GGTGCTGTCTGGGAAGATCTCCCCACACCCCCTCCTGCGCTGGAGAGCGCTCTTCCACCTCTGGTGAGCAGAGGCCCTGGATTGTTTGT
CGCGCTGGATGCAGGGAGATTTGCTCCCTCACGGCCACCATGCACTACCTTGGGCATTGGTGTGGACGGCTCAGCCTGCCTGTGTCCCG
TTACTCTCCCCTCGTCCTTCAGGCCAGGCAGCCTGTGGCCACTCCATGCTGAAAGCGCTTTACCTTGGCCACAGGGCCGCCTCCTTTCT
CCACCCACCTCCAGCCCTTCTTGTGTCCTTAACGAGCCTGAGCTGCAGAGGCCCCCTCCTGGCCTCTCCCAGGCTCGCCCACCTGCCAG
AGCCGCCTCCAGGGGCGGGGAGAGCTGTCGGCCTGCCTGCACCACGTGCTCTGGGCAGCCGACTGCAGGGGTGTCCAGCAGAGGAGCTC
GGCTGCCTGAGGCCCTGCCACGGCTGCCGCCAGCCAGCCGGGCTCAGCTGAGCCCTGAGGGGGCGCTTCACAGCACTCTCAGCTTGGGC
CGCCACCGTGGGCAGCAGAAGCACCCAGTCCTCACTTCCCCTGGCATGGCCCCACAGGCCCCTCCCTGACATGGCCTTGGCCCCAGAAC
CCAGTGGGGACACACTCGCACATACACAGGGTGCCGCCTCCTGCTGTCCCCAGCCCTGCCTCTGACCCCCCTGTGACCGCCTCCTTCCC
TGGCCCAGGAGGCCTGGTTACCTTCATGGGGGAGCATCGCCCCATCCCACCCAGCTCTGCTGTGGCCCACCTTTGCTCAAGCCTCACTT
CTCACATCTGTTTGGGGGCTCACTCTGGGTGACCTAGGCCACAAGGCCCACGCGGCATCAAAGAGGCAGTAGCATCTTCTCCCCTCCCC
AGAGGGCAGAGCCCCCCAAGCCTACTTCAGAGCTCCCTTCTCACACCGGTAGCCCGCAGCCGGTATTCCAGAATGAATTCTGGTTTAGG
CGTGAGGCCTCCCCCACCTCCTCCACCTGCTTGGGGCATGAACCCCTCCCCCACGTTTCCAAGCGAGTCCCCAAGGTGGGCAGATGAAG
ATGCCAAGGATCTCGACCAGTCTGGATGGGTCTGGGGTGGGGGGGCATGCGGCAGACAGGGAGCCATTCTCTGGCTGGTGCTCCTCAGA
GGAGAGAGGCCTCCGGAGACTCCAGACAGCCTTTTATGGAGCTGAAAGTGGCTTCAGAGAAATGCAAAGTTTCCTGGAGAGAACGTGGG
GCGTGGTTCTTGCACAGCCTCCCTACAGGGTGGCTCCAGCAGTGGAGCTCCCCTCCCACGACCCCTGGGTGCTAGTGGGAGGCAGTGGG
CAGGTGCAGATTCTCGTCCTTCCCACTACTGCACACCCTTTGTCTGCGAAGGCGCCCCCAGCGGTGGGTGAAGGAGGAGGGACACTTGG
GGACCCAGCTGTGCACGTGCTCTCAGTGACTGTGGAGTCCACTCCACGGTGGGTCCCGAGGGAGGGGCAGGAGACCAGGGGACCCACCC
CTGCAAAGTGCTCCGGGTCCTGACCCGTGGCCACCCCATGGAACGTAACTGAGCAGCCACTGCCTTGTTCCTGCTGGACATCTGTGGAG
ACAAGAGTGACTTACGGCTGCTTAAAGTCAGAAACAGGTTGAAGGAGGTGGAGGCGTGGGAAAGAGTCTAGGAAGGTGTTTTTGCCCTC
CACGTGCCAAAGGTTACATTTAAAGGTGATGCTGGGTGTTCTCCCTGCACTAGCCATTCCTGGCCCCAGGTCCCCAGCAGGTGTGCACA
TGCTCCATACACTCACGCATGGGGGTTTCAGGGCAGGTGCGCCCTTGGCTCCGTCCGAGGCCAGGTGAGGAACGTCCAGTGCCAAGGAG
CTTCCGGGACAGCTGTCACTTCCCTTTACAACCAGGCAGCGGATAGGGTCAAATCCTGGAGCTTTGGTGTCTAATTCTGGGTGGCTCCT
AATCTAAGCACAGACAGCACCACACACTGGCGTGGGGGCACGAGCTTCTGAAACAACGTGGCCCCAGTGACTCCACGCTGTGTGTGCCC
CTGGAGACGGGGGGCTGCACAAGGTGCGGAGCCAGCTAGAACCTGTCGCTCCCTGCAGAAGCGGTTTCTGTGTGCGGTTCTGATTTGCC
TCAATGAGAAGGTTTTCATTCATCGCTCCCGGCTCTCAGACTGCGTGGAACTGCTCCCATTTAAAGGGGAAAAGAGGTGCCTCGGCTCG
TTAAGGATTTCTTTTTCTAAGTTGTTACGGCGCCCACCAGCCGGCTTTGTCTCCCCTTCAGGGTGGCTGCCTTTCTTCCCCGCCCCTCG
CCCCCGGCCCTCTCTTTAACAAGGCCGAAGTTGTTTATTCTCTCGGGATGAAGTCTCGCATGGGCCGCCACACCCCTGGCGGCCCGTGG
GGGCCCCTCTCCCTTTGTGCCTGGGTCGGCTCCCATTCAGCTCCCCCGACCCCCCTTGTTCCCGGGCGCTCACTCGCGCGAGATGAGGC
GATGGGGCCCACAAAGATGCCACACTCATCCCTGCCGACCTCCGGCTCCCAGCCCAGGGCCCCTCGTTCCTGTGCAGAATTCCTCGTGG
GTGTGACAAAACCCTGCCCCCAGGCTCCGCTGGCGTGGGGGCCAGGCCAAGAGCCACATCCCACACTGGCCCACCTGTCCACCCTAGCC
GCATGACTCCCCTGAGGAGGGGAGGCCGGCATTCCCCGCCACAAACCAGGACGTAATTGGTGGCAGGGCTCTCTGTGGAAAGAGCCAGT
CTGCTGTTTGTCTAGGAGGTCAGTCACAGAGGCCCCGAGACGCCCACTACTGCAGCCTGGCAGGCGGATGAGCCCAGTATCTGGCAGTG
ACCAGAGGGAGTTTTGTGCAGACCACAAAGGCTGATGCCCTGCCCTAGATTGCTCTCCCTCTTGCAAGTGGGCCCAGATCTGCGGGACA
GTCCCCAGCAAGCCCCAGGTCAGGGCACTGGTGCCCTCTTGGGAAAGCTCCTCCCTCCTGGGGCCCGGCTCCCGGCCCAGTCCTCCAGG
GGTGTCCCATGGTGACTGGTGCTAGCAACCCCACACCTCTTCCCTTACTTGGGAAGTCACTGGAATTGTTCGGCTACATCAGACCGCCC
ACAAAACTGTTTTTCTCATCGGCCAGAAATAGGACAGTTGTGAGTACAGGCCCCGGGTGGAGTTGGCGTGTACTTGGTCTGTCCTCTGA
ACCTCACTGTCACAGTCATGGTCCCATGGTAAGGGGCATGGGTTGCTGGAAGAGCTCTTCCTTCCCGAGTGAGCCAAGCCGGGCTCTCC
TGGCGCCAGGCCCTGAGCCGCAGCCACACCACAGCCCCCCTGAAGGCTGCCGGCCAGGGCTTACCCCTCAAGCCACACGGAATGCCTTC
ATCAGTACCCTCCAGCCCCGTGGCCTGGCCCGGGTCGACCCCTAGGCTTCAGCCATGCGATGTCCCTTCAGAATATGACTTCTCTCCAA
TCCCTGCTGCTGGGGGGTGGCAGGTACTTGGGGTCAGGGTTAGGCTCATAGAAGCGACATCTCTACGTCCTCATATTTGCGTCATCTAA
TTTTGTTTTTGTGAATACGTGATAACATTCACAAGGCTCAAGATCCTAAAAGGATGAGAAGGCAGTGATGTCCCCATCACCTGTCCTCT
GTCTTCCCGTGGCTTTCTCTTTCCTTGGTTATGTTTGAGTCAACAGTGGGGCTGACGTTCCACGACGGTCCGTCCGCCAGGCTCTTGCT
CTCCGAGTGCCCAGGGATGCCTGGAGGCTCAGGAGCGCCTGGATGTGGAGCCTCACATACCAAGTGCTTCCCTTCAGCCCGCCCCGCTT
GCTCAGAGCCACCACACAGGGATGCCCGCATCACGGGGCCCCTCACACGGTCCCCTGCTCACAGCCTCCTTCCCTCTCTCCTTCTGCCT
CAGATGTGAAGTTCATTTCCAATCCCCCCTCCATGGTGGCAGCGGGGAGCGTGGTGGCCGCACTGCAAGGCCTGAACCTGAGGAGCCCC
AACAACTTCCTGTCCTACTACCGCCTCACACGCTTCCTCTCCAGAGTGATCAAGTGTGACCCAGTAAGTGAGGGTGATGTCCCAGGCAG
CCTTGCCGGGGCTTACAGGGGGAGACACCTAGTGCCACGGAAATGCCCAGGCTGGTGCCAAGGCCCCCAAGGGTGACAAGGTTGGGGCT
GGGGCTGGGCCCCTCGGACCCCACGCCACAGACTGACAGGGCACCGGCTTCTTCCACTGCTCCTAGAACTTACTGACTGGCTGGGAGGT
CCTCACACCCTTCTCACGTCCCCTGGGGCTTCCAGGAGCCGTAGACTTTCTGGGCGAAGCGTCCGGGACGGAGGCCCCAGGCGGCCCCA
GCCAATGGTCTGTGTGGTGATCGTGTGTGGGGTTAGGCCCAGGCGAGCTTTGTTTGGGCCACAATGTGCGTGCCCAATAAATAGATGCT
TGAAAAGGGCTCCTGTGAGGTCCGAGACACCGGACAACGGGCGGATAGAGACAGCCTTGTTGTTTACGGCCTCTTTGAGAGCCTGCTCC
TGTTAAACCCTGGGATGACTGTGTCTTTCTTCTTAAAAATGCCATTGTTTTATTCCCGAGTCTTTTCTTAAAGAAAGAATTAAAATGAC
AATCAAAAGGGTTTGTGGCATTTACCAAATTAGACCAGAGACGTGGCCGGGTCAGCCGCCGGCCCCCCGGTGTGTGAGGCAGTGACCGC
CTGACCCCAGCTTGGGGCTGGGTGGGCCTGCAAGACCCGTTTTGGCTCTGGCCTGGGCCCCCTCTTGGTCGTCTGCCCTCGAGCCTCCC
GGGGACTCCGCACGGGTCTCACCAGATGCTATCTAGGGTCCACCTGCCTGTCCCCTGCCTAGTGGTCCCTCTGTCCCGGCGACACTGGG
AGTAGCGGCTGCCCAGCCCATGTGTGTCTCGGAAGACGAAGAAGCTTTTTTGCCGTGCGACACCGAAGTTGGCAGGGGCCTCCCTTCTG
TGTTCTCGGCCATGGCCTCCCTTGCACCCTGCCCCGTGTTATCCTTTGGGGGTGGTGAGGTGTCCTCACCCGCTGTAGGGTGGAGGCCA
GCAGCCCGCAGCTCTCTCAGGAAAATGGCTCAGAAACACCATCGAGGCCTCCAGAAGCCCAGCAAAGAGAAAGCCCCTCCATCAAAATG
AAACTCGCGTCTCCACTTTTCATTTCGAACTCCACGCCCTGAGTGAAAACCGCTTCCCCGCCAGGGGTGACTGCCCTGGGATGTTGCTG
TCTTCGGGCAGTTGTGGGAAGTTGGGCGCTGGCCCTTATTTGAGTAGAGACCATCTTAACTAGATTGGAGGCACACGTCTCACAGCTGA
CACACACACGGGGTGAAGTTACCCGAGGCGGAGTCCACTCTGCCTGATCAGCTAGTGACCAACGTAGCTGAGCCCAGACTCAGAAAAAC
CGTCCACAGCAGAGCCCCCTGCATTTTCTAGGGCGTGTTCTAGAATTTTCTTTGGTGGGTGGAATGTCCATCTGTGCAAATCGGGTGCG
CAGTGCCACACACCAGTGACTTTTCGCGGAGGAGCGTGCTGCCTTTTTGGAGCTTCTGGCTGTGGGAGAACAGCTTTGTCCACCCGGGT
AGCCTTCCAGGCAGCTGTGGGGCCAGAGGAATGAAGGAAGGTCCTGGACTCTACCTCCATGTGTGACCCTGGAGTGGGTCATGGGCGAG
GGACGGGCCGCAGGTGAAGAATCCCTGGATGGAGCTGCCAGGCCCCTGGGGCTGAGAATTGAAGCTGGCTGGTGTTTTAGGTTGAACGT
CAGGAGTCTTGTATCTCACCCCAGGCCTCTGGCCTCAGTTTCCCCATCTGTACAGTGGGACTGTTTGTGCAGCCACCCCGGCCAGCTTC
ATTTGCCATGATCAGAATTTATCTGAGGGGCGGGAGAGGAAAGCCCTCCCTATAAAGGTACAGGCGCTAAAATCTCATGACCTCAGTGG
TCCACCTAAAAGTCGTTCTGGCCTGGGTCATCGCCTGTCGTGCTATGCCTTTGTCCAGCCCCTTCTGGTTGGCAGTTAAGTGGCACCTG
TGCGGCACGTGGTGGCGCTGTGGCCCAGCCCTGCTCCTTGTCGAAGGTCTGTTTCCTGGGCTGCCTAGAGACTTGGCTTGAAGCCCTAG
CGTGGCTTCCTGGCAGTTGGGACACACACAGCCCCAACACATGGAGCCGGTTCTCCATCCAGAAGCCCCCGGGCAGTAAGCAGCCACTT
CAGGCTGCGTGGGACTTGCCCGTGGTGGAGCCTAGGAGAGCCCCCTGGCTGGGCGTGGCGTTCCAGATTTCACGGCTGCTCTTTCCCAC
TGACAGTGTGGTGTGGACGCTGCCAAGGGAGTCTGGAGCCCCAGAGCGTGGAGGTGCAGGACTTCCAGGAGCGTCCGTCGCACTCCACC
CGAGGGCGAGCACCTCACTCGCCGCAGTGGGTGGATGCATGCTGTGCCAGGCTGATGGCTGGCCCCCGGCCACAGGCCTGAGCGGGAGA
GGATGGAGGGGAGGGATCAATGGTCCAGGTCCCCCTGGCCACCCAGCATTCATCCTCAGTCATGCACGGCCCAAGGCTTCGACACCCAT
TGATCATGGAAGGCCAGGTTCACCTCAAGGGCTGCCACATGGAGAGGTTAAGTCTGAAAAGGCTGAAAAGGCAGGGTTAAAAGGGCCTC
CTGTCCAGATCAGATGGCACTGAATTCCCCAGGCAGCTGGCACGGCCAGTGGGAACAGGCGGTGAAGGCGCTCTTGGACATGGGGACGG
GCACGCGGTGTGCACGGTGGGCGGGCAAGCATCTGGTGTCTTGTGCCTCCAGAGACCAGGTGGGAGGTGGAGGCGTTTGGTCCTGAGTG
TCCTGACAGGTGATCCCACCTCCCACATCTCGCTCAGGTTCAGAGGAGGCAGCATGGGCCCAGGGACAGTTTTTGGCTTAGTCTTGCTC
TTATAAAGGCTTCCGGGTCATGGCACCTGGGAAGGGGCCCTCGCTGCAGGCCCCTTCTAAGGACCCCCTCTTCCCACCTCTCCCCACCC
TCTCTCTCTCAGCACTGCCTCCGGGCCTGCCAGGAGCAGATCGAAGCCCTGCTCGAGTCAAGCCTGCGCCAGGCCCAGCAGAACATGGA
CCCCAAGGCCGCCGAGGAGGAGGAAGAGGAGGAGGAGGAGGTGGACCTGGCTTGCACACCCACCGACGTGCGGGACGTGGACATCTGAG
GGCGCCACGCAGGCGCCCGCCACCGCCACCCGCAGCGAGGGCGGACCCCGCCCCAGGTGCTCCACTGACAGTCCCTCCTCTCCGGAGCA
TTTTGATACCAGAACCCAAAGCTTCATTCTCCTTGTTGTTGGTTGTTTTTTCCTTTGCTCTTTCCCCCTTCCATCTCTGACTTAAGCAA
AAGAAAAAGATTACCCAAAAACTGTCTTTAAAAGAGAGAGAGAGAAAAAAAAAATAGTATTTGCATAACCCTGAGCGGTGGGGGAGGAG
GGTTGTGCTACAGATGATAGAGGATTTTATACCCCAATAATCAACTCGTTTTTATATTAATGTACTTGTTTCTCTGTTGTAAGAATAGG
CATTAACACAAAGGAGGCGTCTCGGGAGAGGATTAGGTTCCATCCTTTACGTGTTTAAAAAAAAGCATAAAAACATTTTAAAAACATAG
AAAAATTCAGCAAACCATTTTTAAAGTAGAAGAGGGTTTTAGGTAGAAAAACATATTCTTGTGCTTTTCCTGATAAAGCACAGCTGTAG
TGGGGTTCTAGGCATCTCTGTACTTTGCTTGCTCATATGCATGTAGTCACTTTATAAGTCATTGTATCTTATTATATTCCGTAGGTAGA
TGTGTAACCTCTTCACCTTATTCATCGCTGAAGTCACCTCTTGGTTACAGTAGCGTAGCGTGGCCGTGTGCATGTCCTTTGCGCCTGTG
ACCACCACCCCAACAAACCATCCAGTGACAAACCATCCAGTGGAGGTTTCTCGGGCACCAGCCAGCGTAGCAGGGTCGGGAAACGCCAC
CTGTCCCACTCCTACGATACGCTACTATAAAGAGAAGACGAAATAGTGACATAGTATATTCTATTTTTATACTCTTCCTATTTTTGTAG
TGACCTGTTTATGAGATGCTGGTTTTCTACCCAACGGCCCTGCAGCCACCTCACGTCCAGGTTCAACCCACAGCTACTTCGTTTGTGTT
CTTCTTCATATTCTAAAACCATTCCATTTCCAAGCACTTTCAGTCCAATAGGTGTAGGAAATAGCGCTGTTTTTGTTGTGTGTGCAGGG
AGGGCAGTTTTCTAATGGAATGGTTTGGGAATATCCATGTACTTGTTTGCAAGCAGGACTTTCAGCCAAGTGTGGGCCACTGTGGTGGC
AGTGGAGGTGGGGTGTTTGGGACCCTGCGTGCCAGTCAAGAAGAAAAAGGTTTGCATTCTCACATTGCCACGATGATAAGTTCCTTTCC
TTTTCTTTAAAGAAGTTGAAGTTTAGGAATCCTTTGGTGCCAACTCGTGTTTGAAAGTAGGGACCTCAGAGGTTTACCTAGAGAACAGG
TGGTTTTTAAGGGTTATCTTAGATGTTTCACACCGGAAGGTTTTTAAACACTAAAATATATAATTTATAGTTAAGGCTAAAAAGTATAT
TTATTGCAGAGGATGTTCATAAGGCCAGTATGATTTATAAATGCAATCTCCCCTTGATTTAAACACACAGATACACACACACACACACA
CACACACACAAACCTTCTGCCTTTGATGTTACAGATTTAATACAGTTTATTTTTAAAGATAGATCCTTTTATAGGTGAGAAAAAAACAA
TCTGGAAGAAAAAAACCACACAAAGACATTGATTCAGCCTGTTTGGCGTTTCCCAGAGTCATCTGATTGGACAGCCATGGGTGCAAGGA
AAATTAGGGTACTCAACCTAAGTTCGGTTCCGATGAATTCTTATCCCCTGCCCCTTCCTTTAAAAAACTTAGTGACAAAATAGACAATT
TGCACATCTTGGCTATCTAATTCTTGTAATTTTTATTTAGGAAGTGTTGAAGGGAGGTGGCAAGAGTGTGGAGCCTGACGTGTGAGGGA
GGACACCCGGGAGGAGGTGTGAGGACCAGGCTCCCGAGGGGAAGGGGCGGTGCCCACACCGGGGACAGGCCGCAGCTCCATTTTCTTAT
TCCGCTGCTACCGTTGACTTCCAGGCACGCTTTCGAAATATTCACATCGCTTCTGTGTATCTCTTTCACATTGTTTCCTCCTATTGCAC
GATCAGTTTTTTCTTTTACAATGTCATATACTGCCATGTACTAGTTTTAGTTTTCTCTTAGAACATTGTATTACACATGCCTTTTTTGT
AGTTTTTTTTTTTTTTATGTGATCAATTTTGACTTAATGTGATTACTGCTCTATTCCAAAAAGGTTGCTGTTTCACAATACCTCATGCT
TCACTTAGCCATCGTCGACCCAGCGGGCAGGTTCTCCCTGCTTTGGCGGCCAGACACGCGGGCGCGATCCCACACACGCTGGCGGGGGC
CGGCCCCCAGGCCGCGTGCCTGAGAACCGCGCCCGTGTCCCCACAGACCAGCCTGTGTCCCTCTTCTCTTCCCTGCCCCTGTGATGCTG
GCCACTTCATCTGATCGGGGCCGTAGCATCATAGTAGTTTTTACAGCTGTGTTATTCTTTGCCTGTAGCTATGGAAGTTGCATAATTAT
TATTATTATTATTATAACAAGTGTGTCTTACGTCCCACCACGGCCTTGTACCTGTAGGACTCTCATTCGGGATGATTGGAATAGCTTCT
GGAATTTGTTCAAGTTTTGGGTATGTTTAATCTGTTATGTACTAGTGTTCTGTTTGTTATTGTTTTGTTAATTACACCATAATGCTAAT
TTAAAGAGACTCCAAATCTCAATGAAGCCAGCTCACAGTGCTGTGTGCCCCGGTCACCTAGCAAGCTGCCGAACCAAAAGAATTTGCAC
CCCGCTGCGGGCCCACGTGGTTGGGGCCCTGCCCTGGCAGGCTCATCCTGTCCTCGGAGGCCATCTCGGGCACAGGCCCACCCCGCCCC
ACCCCTCCAGAACACGGCTCACGCTTACCTCAACCATCCTGGCTGCGGCGTCTGTCTGAACCACGCGGGGGCCTTGAGGCACGCTTTGT
CTGTCGTGATGGGGCAAGGGCACAAGTCCTGGATGTTGTGTGTATCGAGAGGCCAAAGGCTGGTGGCAAGTGCACGGGGCACAGCGGAG
TCTGTCCTGTGACGCGCAACTCTGAGGGTCTGGGCGGCGGGCGGCTCCGTCTGTGCATTTCTGGTTGCACCCCGGCGCTTCCCAGCACC
AACATGTAACCGGCATGTTTCCAGCAGAAGACAAAAAGACAAACATGAAAGTCTAGAAATAAAACTGGTAAAACCCCAGCGTGGTGCCT
GCCTCTTTGCTTCCTGGGCTGGCCGTGAGCCAGGGACGCGTGTCCTGGTGCCCTAGAACCAGGGCAGGGTCGCAGGCTTGGCGGATGTG
CGACGCCGCAGCCTGTCCTGTGCGCTCTGGGAAGTTCAGCAGCATCCTGACCTCCATCCCCGGGATCACAGTCACGCCACCCGCCGTGA
CAACCAAGAATCTCTCCTGACACTGCCACATCCCCCGGGGTGGGGACAGAATCCAGCCAGGAGCAGCCACACCCCTCCCAACTGGGAGG
AAGCCCTCAGCACAGGTGTGTGAGCTGGGAGGCGGTGTCCTGTCCCCGGGAGGCTCCAGAGAATAATTTGCAGGCTGCCTGGCTGGGTG
AGCCCACCTCCAACCACGCGAGACAACAGCTCCGGCCTGCGTGACGTGAGCGGTGCCCATTCATGGGGAACATCTTCCCCCTCTTCCTT
GCCCCACCAGTTTGTCTTCCCGGGTTATTTGCAGATAGGAAAATAAATAAAGCCGGCATTCGTTAACCCTCTTCTGGCGCAAACTGCTG
TTTGCTCTGGATGAATCATGGTCCTTTGGCCACGCCAGGCTCCGGGAGAGCAAAGCACCGTGTCAGGGCCATGATCCGGGGTGGCCTTT
CACTGGGATCGTGGGGACCTGGAGGCCGCCTTATAGGACACCCATGACGCCCACCTCTGGATTTCAGGTGCACGTGACTGGACTTAACT
TCAAACCCCAGGGTGGAGGCAGGTAGTGGGAGTGCCCTGGGAAGGTGTCCTCGGACCTTGGTCACTGCTCCTGAACCCATCTGTCAGGC
TGGTTTGTCCTCATCCCAAGCTAAGTGGAAGCTCAGGTCCCAAGCCACCGATGGGTCCTACTTGTCAGCTGCAGGTTGAATCTCCGTGG
CCTTTATGAAGCACCTGCTGTCTACCCTTCCTGCCTTGTAGAGCACTCCTCCCAGGGCTCAACAGTGGGGCCGGGGTGGTCGGTGTGTT
GGCTCCACAGGCGCCTGCCCTGGGAGGAAGCTGGGGTGTGGAGGGAAACGCTTGGCCCCTGTAGGTCTCCACCAGCCTCTCCCCTGAGG
GTGGGGGCTCCGGGAGCCTTCCTCGAGGGAGTCCTATATTGACTGGGTGGCGGAGCCTGCAAGGTGCCCCTGACACGTCACATCAGAAA
GAGCTCAAGGGACAGTCGGAGCCAGAGGTGACACTGGTGGCCACTCGGGTGCCTCACAAGGCCCAGCTCCTCCTTGCTCCTGGGCAAAT
TACTCTCAAGGCAGGGACCAGGTCTGCACCATTGCGGCTCTCCAGTTCCAGGCAATGGCCAGGTCCTCTGTCAGGGCTGGGGTCCTAGG
GAAGCCATGTCCCCACCCCCGCCCTGCAGCTGGGTTTACATTCATCCCCCGAGAGCACATGGGTGTAGCAGGAGCCCTGTGCACAGAGC
TCCGACCATCGCACAGGGCACCTTTGGTTGTTTCACGGAGCAGGCAAGGGAGCCATCGGATCCTGTTAGGTTTGAGCAAGGATGTGGGG
AAGAAGCTGGAGACCCACTTTGCCATGCAGGGAGAGGAGCACATGGGTCTAGGGATCTACTTTAGTGTTTGGAAGGTTTTTTAAGATGA
AAGAGGGATGTGTAGGCTGATAGGTCTGGCAGAGCCAAAAGGCAGCGACATGTCTACTGGGAGAGATGGAGCTGAGCGCGGGGCTCAGG
CAGGGTGGCAGGGCAGGGCCGGGGCCCTGGGTGGGTCAGGTGGGTTCACAGCCAAGTGTGTACAGAGGGCTTGGGCCCAGAGTGAAGCA
GTTGCAAGCTCTCCCACAACCCATTCTCTCTGTCTCGGCATCTCTGGCATCCCGTCATGGGTGGGTCTGTACACACCCCACCCCTGGCT
GTGCCACAATGGGGGTGTCTGTACACCCCTCACCCCTGGCTGTGCCACGATGGGGGGGTCTGTACACCCCCCATCCCTGGCTGTGCCAC
GATGGGGGGTTCTCTACATCCCCCAGTGATGGGTGGGTCTATACATCGTGGTTATGCCACGAGCTCCAAGGCTGTATCAGTCCGTTTTC
ACACTGCTAACAAGACATACCCGAGACTGGGTAAATTTATAAAGAAAAAGAGGTTTAATAGACTCACAGTTCTACGTAGCTGGGAGGCC
TCACCATCACGGCGGAAGGCGAAAGGCCCGTCTTCCATGGCAGCAGGCAAGAGAGAATGAGACTCAAGTGAAAGGGGAAACCCCTATAA
AACCATCAGATCTGCTGAGACTTGTTCACCACCATGGGAACAGTATAGGGGAAACTGCCCCATGATTCAATCACCTCCCACCGGGTCCC
TCCCACAACACGTGGGAATTATGGGAGCTACAATTCAAGATGAGATTTGGGTGGGGACACAGCCAAACCATATCAAAGGCATCGTCTGG
CCACTCTAGGCCCTTGTTCCCAACTCTCAAACTCCACTGACATCACCTCTGTGCCTCCAACCCCTCCTGGGATTCAGTGGAGACCTCGT
CCCTTGTTGGAGTATCTGCGCTACCCAGAGGGTCATGGTAAAAACTGACCTTTGAGGAACTTCAGAGACAAACCCTTCCGCAGTCAGCT
CTCCCTGCAGCCCCTTCGCAGAACCCTGTCCTCACACGCCCGCCACCCCTACCTCCCCTGCCTCCTGGCCCATCTCCCTGCACCCAGAC
AGTCCTCATCCATTCTTCATCCTCTGCCCCCAACCCCCAGCCTTCCTATCTCCCTTCAGCTCCGTCCAAGGTCCCTGGGACTCAGCCCC
ATCTCTCCCCAGCCTTGACCCCAATAAGCTCGATGTCTTTGCCCACCAGCTCCTGACACACCAGACTGCATCCAGCTTCCTGTTCTGCG
CTTGGCCCACCGCCTCGGTCCACAGGAACCTTCATCTCTGCTCTGACTGTAAGGATGGGCTCTCTGAGGGCTTCCCCACCCCACAGCAC
GCAGGTGGTTCTGGGACTCTGGGATTGTCATGCCTGGGCACCTTCCTGCTCTGGCTGAGCCCCTCCAGGACCGAAATCTCCCAGATTTC
TCTCTGCCTCCGTCTCCCCACCCTCCCATACAGTGGGTCCTAGGAAGGGGGCCGAATGGAAGGGAGGTCGTGTCGGCTCCTACAGGGCA
GCCCCAAGCTGCTCCGCTGAGGTGCAGAGCCATGGGTCCAGCCTGACCCGCATGCCGGGAGCTGATTCCATGGCTCGAAGGGTCGGGGG
TTCCCCAGGAGCCCAGACAGCACCTAGAGTTGTCACAAGGAGTGAAAGGCCCTGAGGGGACAGAGGAAGGAGGCTGGGCAGAGTGGGCA
CGTGGACTTCGGACCGCGCTTGTGGGATAACAGTGGCTAGGGCTGTGCAGAGGGTGGTGCACTCCCCATGGGAGTCCAGAGAAGCCAGG
TCCAGTGGCAGTGGCATGCATCCGGCAATCCCTCCGGCTACCCTGCATGCAGCGTGACCCTCCTTGAAGCACCCCAGGAAGCAGTCACA
GCCTCGCTCTGCCCAGCATTGGGGGCCTCCCCTCTCAATCTGGTGGGGAAGGCTGTGTTGCCTGCTGGCACCCGCTCTATCACCACTCC
CACCATAAGCCTAGTCCCCAAAGTCAGCACTGCAGCCAGGGCCAGCGAGGGCCAGCAAGGGCACCCCTGTGCAGGGGCTGCAGCGTGGG
GTGTGCGTCCCGCACTCTGGGCCAGCCTCGCAGCTCACACGGCTCTGTCATCCCAGGAATGTCTTTGATCAACACTTTATGCAAGACGA
GGAAAGCAAGCCCAGGACCTCTAATCCGTTCCCACAGTCCCCTGCCCGCCAGGGCCCCACAAGCCCAGCGCCACACCGCCAGGCCTGAG
CAGACAATAGCAGGCGGACGGGTGGGCGGGCAGCTCTGCGGCCGCTGGCCTTGGGGCCCACATCCGCGACCATTGTCACCGCCACTCAC
GAGGGCACACAAGGAGGGCTGGGGAAGAGAGAATGCAGAGCCCTCTGCTGAAGGGGCTTCGGTGGGCCTTGAGCCTTTCAAAGACGGCG
GTGTCCATTAATCCTTTTAATTTGCACCTAGCGGCAGCTGAGACAAGCCTCTCAACCGGCAGCCACTTTAAGAGCTGAGTGGTCTAACC
CGAGGATCAGCTCCTATTCCAGACCCTGGGCCCTCTGAGCCTGTCGAGGCAGTCCTCCTTGCCTTTGTTTAATCACGGGAGGAATCGCA
GCTGGAGGTTACTGAGCACGCACTGTGTGCCTTGTGCTCAACACCGAACACAGTTTCTCTCTGAGTTTTGCAACCACCCCATGGGTTGG
TGACAGGGAGGGGACACTCTGGTGGCAGCCACAGACACAGCCCTGCCCTCCCAGAGCCTACACTCTCCCGCACACAATCATCCCATCTG
CTAGGTCAGGGATTGCCAGGTTTTCTGGAAAGGGCCACAGAGAGAGGCTTCAGGCTTTGTGGGCCACAGGTTCTCTTGCCCTGTGCAAA
AGCAGCTGCAGCCGCTCTGTCAATGAGTGAGCATGGCTGTGTGCCAATAAAGCTTTATTTACAAAAATAGGCACCAGACTGGATTCAGG
CTGACCCTCCTAGTTTGCCGACCCCTGTTCTAGATGAGTAAACCGAGGCCCAGAGAGCTAAAGTAACTGAGCCAGGACTGCACAGCTAG
TGTGTGACCAAGCCAGCCTGGTGGGTCAGCCTCATGGAGAAGAAAAGGCTCAAGCTTCAAAAAGGGAAATGCACGGAGGCTTCATTGGG
CCATTTGCAGCCAGGCCCCACCCACACAGCTCAGGGCCCTGCTTCGCCAGCTCCACCTGTTTCGAGGCTCCCAGTAGGTTGCCCCTGCG
GGCGGCACTGTGTCTTGGCTGCTGGCCTGGAGAAGGGCAGCCAAACAGGCCGGCAGTGGACATCTGAGGGTGACCAGCTGTTTAAAAAG
AAAACTCACCGGCATGTATACGCACACATTAAGCACACACGTTTTCCAGTAAAAGGCCCTGGCGTTGGATCTCGGATCTGCAGCTTTCC
AGCTGTGCGGCCTCATTTCTCTGGGCCTCAATGTTCACACCTATAACATGGGGAAATCCTGCAAGAACACAGCCCATGATGGGGAGTCG
TCTCCATCTCCTACTCACGACAACGACCAGCACAGAACGGGGGCTCCATAAATCTCTGTCAATAACTGAATTCTTTCTGGGGTGCTGAG
GATTCTATGGCTCAATCTCTGTGAAGCACTGCTGAGCACCGAGCTTGGGGTGCAGTGATCACTCAGGGAACGCCATCATCCCCTCGGCG
CATCCGCATAATCACCATCAGCATTCCATCCACTGCCCAGGTCTCGAAGCACAGGCCTCCATGCCACCGCGGCCGCCTGGGTGGTGCCA
CCAAAGCCGATCTTACTTCCCTCCTTTGCCACATTAAGTACACAGTTCTGGGTCTTCTTCACACCACGGATCCCCCCTCCTGGTGTTGA
CAGCACCCAGACTTTCCTCCAGCGAACCACCATTCCCAGATCTGTGCTTTGAGTGGGACCGAACCCATCCCCAGCTCCAGCCCAGGTCT
TCATAGGCTGAAACCCAACTGCACATCCACACCCACCAGAAGAGCAGCAAGTTAATGGATGGGTGAGAAATCAAATAGAGCCATTGAGT
CTTGAGGACACATTTTCTGCAGCTTCTGAAAAAGATAATGCTTCCCCCTTCCTTAGCATTCCCAGCAGGGAAACACAAGCCCAGGGGTG
GCACTGCCCCCCTGCAGCCCTAAGGGAAGACTGAGCTGCTCCCAGGACCTCACAGGGGAGGCTGAGCGTAAGGCCAGCCCTGAGGGAAA
GCAGGGAATGGGTCTCCAGACAAACTGCTGGATCAAGCCTCTCCTGAAGTCATCCCCCTTTTGTTCCAGAAGCCAATCATTGTCCTTCA
TTAAGTCAGTTTCATATGCGCACTGTGTCATTTGGTTTTCAAAGACACTTAACTATTACAGCTTCTGTGTGTTTTCTTTTTTTTTTTTG
AGACGGAGTCTCACTCTCGCCCAGGCTGGAGTGCAGTGGCCTGCTCTCGGCTCACTGCAAGCTCTGCCTCCCGGATTCACGCCATTCTC
CTGCCTCACCCTCTCGAGTAGCTGGGACTACACGCCCCCGCTACCACGCCCAGCTAATTATTTGTATTTTAGTAGAGACCGGGTTTCAC
CGTGTTAGCCAGGATCGTCTCGATCTCCTGACCTCGTGATCCACCCGCCTCGGCGTCCCAAAGTGCTAGCATTACAGGCCTGAGCCACC
GCACCCCCCCGCTTGTGTGTGTTTTCTACCTCTTCTAGATCTAACATGTTATGATTGATATAAACAACAGGAGCGACTGACAAAGTTAC
CATGTGATCTTGGAATTTCAACATGCCTGTCTTGCTTAGCTGGGAGTTCCACAGGGTCAGGGAATGACTCCTCTTGGGGCCACCAGTGC
CCAGAAGTATGCCTAGAACTGCTCTCTCTGAAGGGACAGGTCCCTCTTCCATGGCACACACTCTGTGTGTAACAGAGCACACGTGTGAC
CAAACTGACCTGGAGAAAGGGCCTGACCACATCCATATCCCAAGTTCTGAGGCCGGATAGTGCCTGCAGGGGCTGAGACCAGGGGAGAG
GCTCAAGCCAGCCTTCTCTGGTGCACCCTGACTCAGTCCTCTGTCCACGGCAGCTGCGCCTCCCAGAAGGCAGAGGCCTCTTGGAGAGT
GAGCCTTCCGGTCACAGCGCCTTTCGGGGCCTCCCGCCACTTGCCCTCAGTGGCGCCTGAGACCTGGGGGAGTCCAGCCAAATCCCTGA
GGAGGCCCCACATGCAAGCCCAGAAGTGTCTGGATGGTGTGGCCGGCACCCTTACCCTCCAGACCAGCAAACGAAGCCCAGGCAGGGAG
AGGGGCCTTGTAGTTTTCTGTGGCTGCTGTAATGAATTTTCACACACTCAGCAGCTTAAAACAACCAACATTTCTCTCATAGTTCCAGA
GGCCAGCAGTGTCTGCAGTGCCCGCTCTCTCCCAGCTTCTGCTGGTTTCGGGCGACCTTCGGCTCTTCTCAGCTGGTCCATGTGCA

| | |
|---|---|
| HUMAN SEQUENCE - mRNA | (SEQ ID NO: 17) |

GCAGTAGCAGCGAGCAGCAGACTCCGCACGCTCCGGCGAGGGCCAGAAGAGCGCGAGGGAGCGCGGGCCAGCAGAAGCGAGAGCCGAGC
GCGGACCCAGCCAGGACCCACAGCCCTCCCCAGCTGCCCAGGAAGAGCCCCAGCCATGGAACACCAGCTCCTGTGCTGCGAAGTGGAAA
CCATCCGCCGCGCGTACCCCGATGCCAACCTCCTCAACCACCGGGTGCTGCGGGCCATGCTGAAGCCGGAGGAGACCTGCGCGCCCTCG
GTGTCCTACTTCAAATGTGTGCAGAAGGAGGTCCTGCCGTCCATGCGGAAGATCGTCGCCACCTGGATGCTGGAGGTCTGCGAGGAACA
GAAGTGCGAGGAGGAGGTCTTCCCGCTGGCCATGAACTACCTGGACCGCTTCCTGTCGCTGGAGCCCGTGAAAAAGAGCCGCCTGCAGC
TGCTGGGGGCCACTTGCATGTTCGTGGCCTCTAAGATGAAGGAGACCATCCCCCTGACGGCCGAGAAGCTGTGCATCTACACCGACGGC
TCCATCCGGCCCGAGGAGCTGCTGCAAATGGAGCTGCTCCTGGTGAACAAGCTCAAGTGGAACCTGGCCGCAATGACCCCGCACGATTT
CATTGAACACTTCCTCTCCAAAATGCCAGAGGCGGAGGAGAACAAACAGATCATCCCCAAACACGCGCAGACCTTCGTTGCCTCTTGTG
CCACAGATGTGAAGTTCATTTCCAATCCGCCCTCCATGGTGGCAGCGGGGAGCGTGGTGGCCGCAGTGCAAGGCCTGAACCTGAGGAGC
CCCAACAACTTCCTGTCCTACTACCGCCTCACACGCTTCCTCTCCAGAGTGATCAAGTGTGACCCAGACTGCCTCCGGGCCTGCCAGGA
GCAGATCGAAGCCCTGCTGGAGTCAAGCCTGCGCCAGGCCCAGCAGAACATGGACCCCAAGGCCGCCGAGGAGGAGGAAGAGGAGGAGG
AGGAGGTGGACCTGGCTTGCACACCCACCGACGTGCGGGACGTGGACATCTGAGGGGCCCAGGCAGGCGGGCGCCACCGCCACCCGCAG
CGAGGGCGGAGCCGGCCCCAGGTGCTCCACATGACAGTCCCTCCTCTCCGGAGCATTTTGATACCAGAAGGGAAAGCTTCATTCTCCTT
GTTGTTGGTTGTTTTTTCCTTTGCTCTTTCCCCCTTCCATCTCTGACTTAAGCAAAAGAAAAAGATTACCCAAAAACTGTCTTTAAAAG
AGAGAGAGAGAAAAAAAAAAAAAAAAAAAAAAAAAAAAAAAAAAAAAAAAAAAAAAAAAAAAAAAAAAAAAAAAAAAAA

| | |
|---|---|
| HUMAN SEQUENCE - CODING | (SEQ ID NO: 18) |

ATGGAACACCAGCTCCTGTGCTGCGAAGTGGAAACCATCCGCCGCGCGTACCCCGATGCCAACCTCCTCAACGACCGGGTGCTGCGGGC
CATGCTGAAGGCGGAGGAGACCTGCGCGCCCTCGGTGTCCTACTTCAAATGTGTGCAGAAGGAGGTCCTGCCGTCCATGCGGAAGATCG
TCCCCACCTGGATGCTGGAGGTCTGCGAGGAACAGAAGTGCGAGGAGGAGGTCTTCCCGCTGGCCATGAACTACCTGGACCGCTTCCTG
TCGCTGGAGCCCGTGAAAAAGAGCCGCCTGCAGCTGCTGGGGGCCACTTGCATGTTCGTGGCCTCTAAGATGAAGGAGACCATCCCCCT
GACGGCCGAGAAGCTGTGCATCTACACCGACGGCTCCATCCGGCCCGAGGAGCTGCTGCAAATGGAGCTGCTCCTGGTGAACAAGCTCA
AGTGGAACCTGGCCGCAATGACCCCGCACGATTTCATTGAACACTTCCTCTCCAAAATGCCAGAGGCGGAGGAGAACAAACAGATCATC
CGCAAACACGCGCAGACCTTCGTTGCCTCTTGTGCCACAGATGTGAAGTTCATTTCCAATCCGCCCTCCATGGTGGCAGCGGGGAGCGT
GGTGGCCGCAGTGCAAGGCCTGAACCTGAGGAGCCCCAACAACTTCCTGTCCTACTACCGCCTCACACGCTTCCTCTCCAGAGTGATCA
AGTGTGACCCAGACTGCCTCCGGGCCTGCCAGGAGCAGATCGAAGCCCTGCTGGAGTCAAGCCTGCGCCAGGCCCAGCAGAACATGGAC
CCCAAGGCCGCCGAGGAGGAGCAAGAGGAGGAGGAGGAGGTGGACCTGGCTTGCACACCCACCGACGTGCGGGACGTGGACATCTGA

TABLE 4
(mouse gene: Myc; human gene MYC)

| | |
|---|---|
| Mouse genomic sequence | (SEQ ID NO: 19) |
| Mouse mRNA sequence | (SEQ ID NO: 20) |
| Mouse coding sequence | (SEQ ID NO: 21) |
| Human genomic sequence | (SEQ ID NO: 22) |
| Human mRNA sequence | (SEQ ID NO: 23) |
| Human coding sequence | (SEQ ID NO: 24) |
| MOUSE SEQUENCE - GENOMIC | |

(SEQ ID NO: 19)

CTTCCTTCTCCCTTCACTGAAACTGAACTGACCTTGGGATGGGGAAACCCCCACTTCATGGCACCCACTCTAAAAGATGGCTGTTGGTC
AAACAGCCTAGCAAGCTCTAAACCAGGTACTCTGCACCTGCCCTCTGACGCCCTGGGATTATATGGCAGAGGGTAAGAACAGGAATGAC
ATAATTAAAACCAGCCTCAGAAGCTGCTGAAGGCATTAGAACTAAAGCCCAGAAAAGCAACAAACTGTTACCATCAAAGCGTGAAAGAG
GAAACGGTTAATCTTTTCAAGGGAAGTGAAGCAACTCGGAAGGCCATAGGAATCCACATCACCTGAACTGCAGACCAAGCCTTGGAACT
GGGGCTTTCAAGCTACATCTGCTACTCACACCTTTGACCTCTCTGGGTGAATTGTTTTCTTACTAGTCACCAATGTTTTCAAACCTGGA
TTTTCACACTCCCTGCAATCCCTGGAAGGGCTCCTACCACCGGACCTATATTGCAGGGCCACCAACTGCCAGGTTCTCAGCCACAGCTC
TTAGCTAGGACCAAGCAAATGGTCCCCAGTGTGTTACAGTCTCCTGTCCTCCAAAGTGAGTTCCTCAGATGCACTCCTTTCCAATCCTN
NNNNNNNNNNNNNNNNNNNGATTTTCCTGTGGGCACATCAGCTTCCTATGCTGAAAAAAACAGAGAGTAGTAGAAAGGGAAAGCCTAAT
CAAGCCTGCAGCTGAGAGATTGCCCTGTGCCACAACCCCCAACTCCACCCCCTTATAGGACAGCACCTTACTCATTCAGCCAAAAGCTT
AATGGATTGCCTTGATTCGCCTTGATTCCATAATGTAATTGACCATGAGTAGAGCAAGCTGCACCAACTCCTATCGTAAATGAAGACAG
GGGTGAGACTGGAAGGGTCAGATACTGGAAAATCTGAGATTCTCTGCCTTGGTTTCTCATCTTGATCAGTTTCAACCAAAAAAGAAGGA
ACCTTTAACCAGGACTCCTTGTTTTTCTCAAAAGAATATGCTAAACTACACATTCCCATTATTGCTCATGAGACCTTGTTCATATGTCA
CCAACAATATTCACTGGTATATGTTTGTTCATACCTTCTTAAACATAATTTAACATTTCTTTATTTGATGTATGTGTTTTCCTTGCATA
ATGTATATGTACCACGTGTGTCCTTGTACCTGCACAGGTCACGAGAAGGTTTCAGTTTCCCTCGATTGATGACTGTGAACTATCATGTG
TATGCTGTGAACTGAACTAAGGTCCTCTGTGGAAGCAACAAGTGTTCTTAACTGATGATCCATCTCTCCAGCTTCCGCTCAGAAATTTT
CACACTTAAAGAAATAAGGGCTGGCTGGTGAGATGGCTCAGCGCTTAAGAGCACCGACTGCTCTTCCAAAGGTCCTGCCTTCAAATCCC
AGCAACCACAAAGAAATAAGGGCAGGGGCTAGAGAGATGGCTCAGCAGTTAAGAGCACTGACTGCTCTTCCAGAGGTCCTGAGCTCAAT
TCCCAGCAACCACATGGTGGTTCACAACCATCTGTAATGTGATCTGATGTCCTTTTCTGATATGTCTGAAGACAGCTAAATTTTACTCA
TATACATAAAATAAAGAATTCTTTAGAAAGAGAGAAAGGAAGGAGGGAAGGGAGGAAGGAAGGGAGAGAGAGAGAGAAAGAACGGAAGG
GAGGGAGGCAGGGAGGGAGGGAGGGACGNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NUNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNCCCTCCTCCTAGCTTTCTGAAGCCAGTCTTCT
CCGGTTTCCCTTCAGAACAAGATGTAAAACTCTCAGCTCCTTTAGCTCCATCCCCGGACTGGATGCTGCCACACTTCCTGCCTTGATGA
TAATGAACTGAACCTCTGAGCCTGTAATCCAGCCCCAATTAAACATTGTCCTTTATAAGAGTTGCCTTGGTCATGATGTCTCCTCACAG
CAATGGAAACCCTAACTAAGACAGCAGTTTAGCAGTAAATGGGGTGGCAGAGACAGGGTGATAGCAGGAAAATACTTTGACATAGCTAG
TGAGACAGCAGGTAATGACAGTAGCCAAACCAGCCTGATAAACAAACAGCTAGCCCAGAATGCATAGCACAGCAGCAAAAACATTGGAG
AACTGGCTCAATGACAAAGTGGAAGGAGGAAAGGCAATTCCTAAATGTTGTCATCTGATCACCATATCTGCACACTGAAGCACACACAC
ACACAAATAAACAACACTTTAAAGAAATAAATATTACATGATGATTGCAACAACCACAGGCCATCGGAAAAGAAAGAGGAAAAAAAATC
AATGGAGCAAAGAGACCAGGCTGCACACACAGGAGGAACATCAGGAGACCCAAATTCACCCAAATGTTCTCAATGTCAGCATGTTTGCA
TCTAGGTCCCACACTCTAGGGGATTGGCAGGATCCCTGACCCCGACTCACTAAATACCAATAGGAAGCCCCAAAACCCCCACCACCTAT
AACAACCTCAAATATCTCCTCCTGTTGGTCAATGTCCCGTGGAAGCAACATTACCTCTAGTTGAGAGGTACTGATCTCTAGAATCCAAA
AGTGTCAGCATTTCCAGAAGCTTTCCCAGCAAACCAACAAAAAGAGATGOTCTCACGGTCACCGGTTGTGGGGAAGATAGCTTGAGTGC
CTTTAGGGAGGTTCATTCAATGATAAAGTCCACATACAGTAGTCAGGAGGGAATGGAGAAGGTAAAACTGAGACCAAGCAAAGGCTGAG
CTGCACTCAAATTCTACCTACAGAAGTCAAGAGCTGTGACCCATAGTTTGGGGTCTGTTCTAGCCAATAAAGACGCACAGGCTACTATT
GCACTGGCTTCTAAATGAAAGTCAAAGATTACGGACAGAAAAAAAGGAAGAAAAAAAAGTAAAAAAGAAAGCAAGACCCGTCATGCACC
TCCTCCCCAGTCACCTTTACCCCGACTCAGCAAGGGTCCATCATCCAGACAGATGCCGCCCAAAAGGACCATCACAAAGGCCGGCCAGT
GAACAAAAGTGCAAGATTGACTTGGGTTTGCCATTTACAGAACAATAAACGAAGCCTTTGAAGCACTGTTAAACAAAAGAGCCATTTGT
CTATGCTGGTGGCTACAAAGGAGAACAGGATTGCAGCAGAGGACTAAGAAAAGAAAGAGAAAATGCCAAACTGAGATGTGCCTGGACTT
TACCGGGTCCTGCCCAGAGACCTTATATGCCAGGACCTAAGATCTAAAAGCCACTGGGTAAGAAAACAATCAAATATGCTTAATGCAAT
ACAGGCAATGTAGGTTCTTGGGTATTGTGTTAAAAATATCTACTAATTTTTTTTCCTTGTCAGCTGCTTTAATTTATGCAGTATGGGAA
GGGATCACATCGGAAAAGACAAGGGAAGAACAGAAAAGAAACACCGTACATTTTCAAATGCACAAAGATCTCATGCACTGCCTTCTGTT
TCTGTGCCTATGAATTAGTGATGCTAAATTTTATCTTATTTTCTTGCCTGGTGCGGCGGGCGGGGAGGGCGGGGAGGGTGACTGTGTTC
CTCTCTGTGGTGGCATAGCTGCCATTTACATGCCTGTGAGGTCCTCTTTTCCTTTAGCATGCCATGGCTAGCTTGGTTTTTAATCTAAC
AGCGCTTCTTTCCAAATGATGTTTCTGCCGCAGACAGGTATTTATCATTGTCTTTGCTCTOGTCACTTCCCGACAGCACTCGTGAGAAC
TGAAGTAATTTTGAAATCGTAGCCCGTCCTTAGGGTCTCCTGCTTCTCTCCCGAGTAGGAGCTAGGTTTTTGATCCAGTTTGTGTCGAT
TTAGTGACTGTTTTTATACATGCAAAAGGTTAGCTCAGTGGTGATGGTTGCGGGGGGGGGCTCTTTGGCATGCAGAAAAGAGATTCAAG
AACATTCGGGGATGTTATCTAGTGGTTTTCTCCTTGTTCTTACAAGAGTGACTTTCGAGAAGAGGGGTATACTTTGGCTCACAGTTTGA
CGGAAAAATCTGGGATCGAAGAAGTCATTGTGGGAGCAGCTGGAAGCTTTGTCACTTGGGGAGCCTTGTCATACTCTAGCCAAAGTTAG
GAAGCAGGTTGGAGGGTGGGGAGACAGATTCTCGAGTTTGGCGCTTCTGTTTTATACCTTCTCTACCCACATTGTGAATAGACCTGACC
CCTGGGTTGACACACATCACATTAGTGTGAGTACCTCTTCCTTCTACACATATACCTCTCTACAAATACCCTCATAGGTAACACCACAT
CTGCATCTCCTAAGTCATCAAGTCGACAATCACACTAACCATCATAGGCAGGGCTGCAACACACCTTGAATCCCGTGACTTAAAGACGC
AAAGACAGGTGGCTCTCTGAGTTCAAGGCCTGCCTGGTCTACAGAGTTTCAGGACAGTCAGGGAAACCCTACCTTGAAAAACAAAAAAC
AAAAAACAAAAAACAAAAAAAAAGAAAAGAAAAGAAACACCCAAATTAAGCGTCTCAAGGATGACCGTTCTTGGTATGTTATTCACACC
ACATATTTGGACCTCCCCTCTTGTCTCTTTGCAAAGTAAGATGTTGGCCCGACTTGTTCATTCTAGCTGGCAATATACAGTTCAGTGAC
TGAATGAATGCTGACCACAAATCAGGCCCTGTTTCAATACTGAGCACACCTTCCTCTCCTTCCCATAACAGCCCTTTCTTTCTGGAGGT
CTTCCTTTTTTTCTTAAAAATGTAAGAGCATCATCACTGTTAGTATGAGACAGTCACGTTCTGCGCACTGTGATTCTTACTTTTTCATA
CTGTAAGAGGGAATCTACTCTCTGCCATCGGGACACCCAGTGGAACTGCTCACCTGGAGTCTTCCCTCCACGAAGACTAGGATCTTCTG
GAAAGCTGTGTGCTTTGGACATCTAACTCACATCCAGCAATAGCTCCCAGAAAAGGACCTCAGAGTTACAACGTTCCAGAGGAACAAAA
CAGTGGGACATCTTCCACCATCTTCCAAAAGGAAACCTCAGAACAGAACAAGCAACCTTTGTCCTTAGTAAGCTTGTCCACAGAGGCAC
GCATGGCTGTCATCCGAACACTGGGAAGTCCAAGGCAAGCGAACAGCAAGTTCAAAACTAGCTTGAACTGGCTGCATAGTGAGGCCCTA
TCTCAAAACATCACCCGCTAACTATGTACCTCACAGACAAGCCCTTGTCCACTGTTTGAAGACTCTGGATTTGATTCCCAACAAAATAA
AAACAACAACATACACTCACATACTCACACACACACACACACACACACACACACACACACACCCAACTCCTTCACTAAAAAAATTTAGT
GTAGGTAGAGTGGCACAGGAAACTCTAAATTCCAGCACTACACCCTGAAGCAGTTTTTATTTTAGTTAGTAACCCATCCTAGGCTACCT
TCTCAACAAGAACATTCCAATCACTACACTTTATTGACAAGATTATAAACTTGTGGCTTTCCTGTCCTTTTTAAAGTATTTTTTAAAAT
TTATTTTACAATGAACTGCTACCCTTTCATTTCCTAATATGTATGGGGATGTCCCCTACCTTCCTCTTACTTAAAAAAGCAAACACCCC
GGGTGATGTCATCAGGCTGGGGTACAGTACGGGCAAAGTCCCCCCACAGGCTATTCCAACTACCTCGGTTCCCACACCTACACTAAAAC
CGGCATCATTTGGACGTAAGATCTTAAAATAATCCATAATTACACCTTTTCAAAAAGCACCTTTTGAGCGTCACCTCTACCCACTGTCA
TATCTGTTGGTGCTGTCCTGTGTTTAGGGAGAGGCAGAGAGAAAGCGGGTGTGCGCAACCTCTAAGAGAGTAATAGTAAAGAGACAAGC
CTACGAAATCTTCCCCAAGAGGCCTTTTGGGGAAGGCTATCCCCCCTTCCGTGGATGTTGAATACCTTGCCTGTTGCTCAGATGCAGTC
AAGCATCTGATGATGCTTGAGGCATGATGCTGACACCACTGTGTAGGTTGCTACAAATTCCCTTTCTATTATACCCACCCTTATGCAGT
TATTTGGGTTTAAATTTTCAACGTTTTAAAAAAAAAAAAAAAAAGTATTGCCGGGCAATAGTGACGCAGGCCTTTAATCCCAGCACTCG
AAAGACAGAGGCACGCACATCTCTGAGTTTGAGACCAACCTGGATGGTCTAAGGCCTTGAACTCAAAAGATCTATCCACCAGCTTGTAT
CCCATTTAATTGAGTGATTTGTTTGCTCTCTCTCTCTCCCGCTCCCCCTCTCTCCCCCCTCTCCCTCCCCTTTTTCAGTTCTTTATTTA
TATTTGAATATTATCTCTCTCTCAGATGTCTACTTGGTGACCGTCTTTTCTACAAAGGATTATCTCTTTCATGACACGCCATTTATTAA
TCTCCTTGTCCCTGGAGCTTCTGTACAGTAGCCTCACCTGATCCTCTAGCTTCTCCATCCCAGATGATTGGGATTCAACGCATTCATCA
CCACGCCTACCTAAAATTCCAACCTTTTAATAAATGGCTCTCTTAAAATATTTTGGCAAGGAATCTCACTCTTTCTCTTCACTAAATAT
AGTTCAACCACCCAATTCGTAACCACTCGGGCTGAGGGTGAGCATCCTTAAGAACATAAGCAAAAAATTTGCAGGCTCAAGATTCAGTT
TAGTTGCTAGAGGGCTCACATAGCATGCCCTCCCCACCCCCGATTCCATTCTCATTTATCGAGGCATAAGGCCAGGTGTGGTGGGATAT
GTGCTGGGATGCATAAGATCTTTTATAAAGAAGAGGAAGAGGAAAAACAGTTATACAAAACTAATAAAACTGTGTGAGTTTCAGGCTAG
CAAAGAATAGTCGTGAGAATCTCTCTCAAAAAAAAAACCCCAAAATAATAATAATGATAATAATGATAATAATAATAATAATAATAAAA
CAAGCCAATAATAAGCTAATGCTCCTGCCTTGCATTCTGACTCCTTTTGCCCAGTTAAATTCAATGCTCTGCTTTGACACGTCCAGCTT
ACAACACGACAAAGGTGTGAGACATGCGTGCTAAAACTTGTTCTATTGCCTTTCCGTTTCTGTTAGATCTCCTTCACTTGTATACCTGT
GACTCATTCATTTTCCCCATCCACAACTAGGGCTCTGTTTTGGGTGACTGTCAGAAAGTAGGGATGGGTTCATCTACTCCCCCTCTTTG
CAACTGAATAGCCACCTCTGAACCATTTTTTTCTCTAGTAATTTCTCTTCCTTTGCCTCCTTTGCAGCCTAA3AACAGTCATTTAAGGA
TGACCGGAAGCTTGTCTTAGGCAAGGAAGCATCTTCCCAGAACCTGCAAACCCTGCAGCCCTGCCCCCATCCGACCTCCGCCCTCGTTG
GCTTCGCAACGCTGTGGTCTCTGTGGCCAGTAGAGGGCACACTTACTTTACTTTCACAAATCCGACAGCCACAACCCGGGTGGTGGGGG
GTGAGGGGGCGGGGAAAGAGTCTCTGCAGCAAAACGCAGACTAGGGATTGGTGGCTCTTGGTGTTTGAGGCAAAATCCTAGAGGCTGTA
GTCATTTTGCAATCCTTAAAGCTGAATTGTGCAATGAGCTCGATGAAGGAAGATACTATCATTCAACAGCTGAATCCTAAATTGCAAAC
TCAGTGGCTAATAACAACTTTGAACAATGAGCACCTTATACACGCTACTGTATTTTCTTTTCTTTCTTTTTTTTTTTTTTTTTTTTTTT
TAAACCGGGTAGCAGTGAGAGAGGTTTCTTTAAGTGCCTTGGGCCGACGAGTCCGGAATAAGAAGACTTCTTTGGGTTTTAAAGTGTAG
GATAAGCAAATCCCGAGGGAATATGCATTATATAATAAATCTAGAACCAATGCACAGAGCAAAAGACTCATGTTTCTGGTTGGTTAATA
AGCTAGATTATCGTGTATATATAAAGTGTGTATGTATACGTTTGGGGATTGTACAGAATGCACAGCGTAGTATTCAGAAAAAAAGGAAC
TGGGAAATTAATGTATAAATTAAAATCAGCTTTTAATTAGCTTAACACACACATACGAAGGCAAAAATGTAACGTTACTTTGATCTGAT
CAGGGCCGACTTTTTTTTTTAAGTCCATAATTACGATTCCAGTAATAAAAGGGGAAAGCTTGGGTTTGTCCTGGCACGAAGGGGTTAAC
GGTTTTCTTTATTCTAGGGTCTCTGCAGGCTCCCCAGATCTGCGTTGGCAATTCACTCCTCCCCCTTTCTGGGAAGTCCGGGTTTTCCC
CAACCCCCCAAATTCATGGCATATTCTCGCGTCTAGCGCCTTGATTTTCCCCACCCCAGCTCCTAAACCAGAGTCTCCTGCAACTGGCT
CCACAGGGGCAAAGAGGATTTGCCTCTTGTCAAAACCGACTGTGGCCCTGGAACTGTGTGGAGGTGTATGGGGGTGTAGACCGGCAGAT
ACTCCTCCCGCAGGAGCCGGGTAGAGCGCACCCGCCGCCACTTTACTGCACTGCGCAGGGAGACCTAACAGGGGAAGAGCCGCCTCCAC
ACCACCCGCCCGCTCGAAGTCCGAACCGGAGGTGCTGGAGTGNNNNNNNNNNNNNNNNNNNNNTGACGCGGTCCAGGGTACATGGCGTA
TTGTGTGGAGCGAGGCAGCTGTTCCACCTGCGGTGACTCATATACGCAGGGCAAGAACACAGTTCAGCCGAGCGCTGCGCCCGAACAAC
CGTACAGAAAGCGAAAGGACTAGCGCGCGAGCAAGAGAAAATGGTCGGGCGCGCAGTTAATTCATGCTGCGCTATTACTGTTTACACCC
CGGAGCCGGAGTACTGGGCTGCCCGGCTGAGGCTCCTCCTCCTCTTTCCCCGGCTCCCCACTAGCCCCCCTCCCCAGTTCCAAAACCAG
AGGGCGGGGAAGCGAGAGGAGGAAAAAAAAATAGAGAGAGGTGGGGAAGGGAGAAAGAGAGATTCTCTGGCTAATCCCCGCCCACCCGC
CCTTTATATTCCGGGGGTCTGCGCGGCCCAGGACCCCTGGGCTCCGCTGCTCTCACCTCCCGGGTCCGACTCGCCTCACTCAGCTCCCC
TCCTGCCTCCTGAAGGGCAGGGCTTCGCCGACGCTTGGCGGGAAAAAGAAGGGAGGGGAGGGATCCTGAGTCGCAGTATAAAAGAAGCT
TTTCGGGCGTTTTTTTCTGACTCGCTGTAGTATTCCAGCGAGAGACAGAGGGAGTGAGCGGACGGTTGGAAGAGCCGTGTGTGCAGAAA
CCGCGCTCCCGGCCGACCTAAGAAGGCAGCTCTGGAGTGAGAGGGGCTTTGCCTCCGACCCTCCCGCCCACTCTCCCCAACCCTGCGAC
TGACCCAACATCAGCGGCCGCAACCCTCGCCGCCGCTGGGAAACTTTGCCCATTGCAGCGGGCAGACACTTCTCACTGGAACTTACAAT
CTCCCACCCAGGACAGGACTCCCCAGGCTCCGGGGAGGGAATTTTTGTCTATTTGGGGACAGTGTTCTCTGCCTCTGCCCGCGATCAGC
TCTCCTGAAAAGAGCTCCTCGAGCTGTTTGAAGGCTGGATTTCCTTTGGGCGTTGGAAACCCCCGTAAGCACAGATCTGGTGGTCTTTC
CCTGTGTTCTTTCTGCGTCTTGAATGTAGCGGCCGGTTAGGACACTCTTTCTTCCATTCCTGTGCTTTTGACACTTTTCTCAAGAGTAG
TTGGGGTAGCCTGGCCTAGATCTCAGTCGGGCTAGAGCGACTTGTCAACATGACAGAGGAAAGGCCAAGGGAAAAACCGGGATGCATTT
TGAAGCGGGGTTCCCGAGGTTACTATGGGCTGACGCTGACCCGGCCGGTTCGACATTCTTGCTTTGCTACATTAATTGATATGTGTCCT
TTGAGGGGTCAAACCGGGAGGTCGCTTCGTGGTGGCCAAAGAAAGCCCTTGGAATCCTGAGGTCTTTGGAGAAGGGATTACCTTTTGCG
TTTGGGAGCGAGAAGGCTCCGTAGCTTCTGACTTACCAGTCTCTGAGAGGGCATTTAAATTTCAGCTTGGTGCATTTCTCACAGCCTGC
GACCGACACGGAGGTGCGTCCCGCCCGCCAATCCCCGGCGGCCATCGCAACCCGTCCCTGAGCCTTTTAAGAAGTTGCTATTTTGGCTT
TAAAAATAGTGATCGTAGTAAAATTTAAGCCTCACCCCCGCGGCACTAGGACTTGATGTTGGGCTAGCGCAGTGAGGAGAAGCAAAATT
GGCACAGGGATGTGACCGATTCGTTGACTTGGGCCAAACCAGAGGGAATCCTCACATTCCTACTTGGGATCCGCGGGTATCCCTCCCGC
CCCTGAATTCCTAGGAAGACTGCGGTGAGTCGTGATCTGAGCCAGTTCCGTACAGCTGCTACCCTCGGCGGGGAGAGGGAAGACGCCCT
GCACCCAGTGCTGAATCGCTGCAGGCTCTCTGGTGCAGTGGCGTCGCGGTTTAGAGTGTAGAAGGGACCTGTCTCTTATTATTTCACAC
CCCCTCCCCTTTTATTTCGAGAGGCTTGTGATAGCCGGAGACTGAGCTCTCTCCTCCAAGTCAGCAATCGGAAAGAAAAGCCGGCAAAG
CCCCTCCCCTTTTATTTCGAGAGGCTTGTGATAGCCGGAGACTGACCTCTCTCCTCCAAGTCAGCAATCGGAAAGAAAAGCCGGCAAAG
CAAGCAAGGGGGCGCGCTGGGCGTGGAGAAAGAGGAGGGCGGAGAGGGGCGGCGCCGCCGGCTGGGTACGAGCGCGGCGACGGCGCGAA
TAGGGACTCGGACCCGGTCGGCGGCCCAGACAGCCGGCACACGGGAGGGGGCCGAGCGACGCGGCGCCTCTCGCCTTTCTCCTTCAGGT
GGCGCAAAACTTTGCGCCTCGGCTCTTAGCAGACTGTATTCCCTACAGTCGCCTCCCTCAGCCTCTGAACCCAAGGCCGATCCCCATTC
CTGGGCGTCTCCAGGGCTAAGTCCCTGCTCGAAGGAGGCGGGGACTCGGAGCAGCTCCTAGTCCGACGAGCGTCACTGATAGTAGGGAG
TAAAAGAGTGCATGCCTCCCCCCCAACCACACACACACACACACACACACACACACACACACACACACACACACTTGGAAGTACAGCAC
CCTGAACGGGAGTGGTTCAGGATTGGCGTACAACGCTGCCCCAGGTTTCCGCACCAACCAGAGCTGGATAACTCTAGACTTGCTTCCCT
TGCTCTGCCCCCTCCAGCAGACAGCCACGACGATGCCCCTCAACGTGAACTTCACCAACAGGAACTATGACCTCGACTACGACTCCGTA
CAGCCCTATTTCATCTGCGACGAGCAAGAGAATTTCTATCACCAGCAACAGCAGACCGAGCTGCAGCCGCCCCCGCCCAGTGAGGATAT
CTGGAAGAAATTCGAGCTGCTTCCCACCCCGCCCCTGTCCCCGAGCCGCCGCTCCGCCCTCTGCTCTCCATCCTATGTTGCGGTCCCTA
CCTCCTTCTCCCCAAGGCAAGACGATGACGCCGCCGGTGGCAACTTCTCCACCGCCGATCAGCTGGAGATGATGACCGAGTTACTTGGA
GGACACATGGTGAACCAGAGCTTCATCTGCGATCCTGACGACGAGACCTTCATCAAGAACATCATCATCCAGCACTGTATGTCGACCGG
TTTCTCACCCCCTCCCAAGCTCGTCTCGGAGAAGCTGGCCTCCTACCAGCCTCCCCGCAAAGACAGCACCAGCCTCAGCCCCGCCCGCG
GGCACAGCGTCTGCTCCACCTCCACCCTGTACCTGCAGGACCTCACCGCCGCCGCGTCCGAGTGCATTCACCCCTCACTGCTCTTTCCC
ACCCGCTCAACGACAGCAGCTCGCCCAAATCCTGTACCTCGTCCGATTCCACGGCCTTCTCTCCTTCCTCCCAACTCGCTGCTGTCCTC
CCAGTCCTCCCCACCCGCCACCCCTCACCCCCTAGTCCTGCATGAGGACACACCGCCCACCACCAGCAGCGACTCTGGTAACCTACCCC
ATTCACAGCAGGGTAGGAACCGAGAGGTTCGATCCACCTCCTTCTCCACCACTCATTGGCATTAATTCAATTGGCCTCCGGGGCTCCCC
CTTTCTTTCCCTTCTGTCTAACACCTCTTCATCCCTGGATTCCCGTCNNNNNNNNNNNNNNNNNNNNNNNNNNNNNNNNNNNNNNNNNN
AANNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNTCAGGANTTCATTTGGGTTTTTAAATCTTCTGGCTTATCTTTCAGCTCCATCCA
CTCCCTTTACCCCTCCTAACCATTTTAATTACCCTGCCAAGGGTGTGAATGACCATAAACCAACTGATCTGGAGGGGGGTGAATTACCT
GCTTCTTTTCTTGACTGCCAGAAGAATATTTGAATTTAATGCATACGTTTAATCTAAGACCCAAGAGAAGCATCACAGAACCTGGGACA
CCCTTTATACCCTTAGAGCCACGCACTAGTGAAAGTTCCTAAAGAATTGAACACCTGCGCTCTTTTGGGTGTTTTGTTTTGCTTTGTTT
TTTCGTCCTCTTTTGAACACTTCAAAGCAAATTCTGTTCAATTTCGACTTCTCCCCCCGTCCCAACACTCCCCCAACACCAGGACGTTT
GCCAAAGCTGCAAGACTTTTTTTTTTTTTTTTTTTTTTTAATTGTGCTTCCAGTAAAATAGGGAGTTCCTAAACTCATACCAAGACATT
TGCAGCTATCCCTCACGGGACCTGAAAGGTTCTCGGTAAATCCCTTAAAAATAGGAGGTGCTTGGGAAATGTGCTTTGCTTTGGGTGTT
GTCTCAAGCCTCATTAAATCTTAGGTAAGAATTGGCAAGGATACCATATCCTGGTACATGGTAATTTTCTCACCTGTCCCCTAACCCTG
TTCTCCCTTTCTGCGAGAAGGGAAGATCGTGTCTGGATCTGATTCTTACTTTCTTCCCTTTCCAACTTGGTATTTGGATAGCATCGGTC
AAATCCTATGTATAGCGTCCGGGATTCAGGAGCAACGTGGCTAACTGTGATCTTCCACTTCCTCCCTTACAGGAAGAGCAACAAGATGA
GCAAGAAATTGATGTGGTGTCTGTGGAGAAGAGGCAAACCCCTGCCAAGAGGTCGGAGTCGGGCTCATCTCCATCCCGACGCCACACCA
AACCTCCGCACAGCCCACTGGTCCTCAACAGGTGCCACCTCTCCACTCACCAGCACAACTACGCCGCACCCCCCTCCACAAGGAAGGAC
TATCCAGCTGCCAAGAGGGCCAAGTTGGACAGTGGCAGGGTCCTGAAGCAGATCAGCAACAACCGCAAGTGCTCCAGCCCCAGGTCCTC
AGACACGGAGGAAAACCACAAGAGCCGCACACACAACGTCTTGGAACGTCAGAGGAGGAACGAGCTGAAGCGCAGCTTTTTTGCCCTGC
GTCACCAGATCCCTGAATTGGAAAACAACGAAAAGGCCCCCAAGGTAGTGATCCTCAAAAAAGCCACCGCCTACATCCTGTCCATTCAA
GCAGACGAGCACAACCTCACCTCTGAAAAGGACTTATTGACGAAACGACGAGAACAGTTGAAACACAAACTCGAACAGCTTCGAAACTC
TGGTGCATAACTGACCTAACTCGAGGAGAAAGCTGGAATCTCTCGTGACACTAAGGAGAACGGTTCCTTCTGACAGAACTGATGCGCTG
GAATTAAAATGCATGCTCAAAGCCTAACCTCACAACCTTGGCTGGGGCTTTGGGACTGTAAGCTTCAGCCATAATTTTAACTGCCTCAA
ACTTAAATAGTATAAAAGAACTTTTTTTTATCCTTCCCATCTTTTTTCTTTTTCCTTTTAACAGATTTGTATTTAATTGTTTTTTTAAA
AAAATCTTAAAATCTATCCAATTTTCCCATGTAAATTGGGCCTTGAAATGTAAATAACTTTAATAAAACGTTTATAACAGTTACAAAAG
ATTTTAAGACATGTACCATAATTTTTTTTATTTAAAGACATTTTCATTTTTAAAGTTGTTTTTTTCATTGTTTTTAGAAAAAAAAATAA
AATAATTGGAAAAAAATACAATTGGGCCAACTTGTGTTTTCTTTTTCCTCTTCCTCAAACTTCCTTTCCTCAATTACAGATTAAAGAAT
TTCACCATTTTCACAGGGTAGGTTTACAAATATGGGAAGGGGTTATCATTGTTAAAATGGGGCTGGGGGTCCTCAGGATTTCTAAGTTG
TCTACAGGATGCTTTCTGTGGATAGTAATAAAAACCAGAGCTGTTAGTTAGGAATGGGCAAAAGGCAAGTGAGAAGGCTAGATGCAGGG
AAGGGAAAAGCAAGAGGTTAAAGATAACAGCTAAATATACAGGAGGAAGAGATGGCAGAATCTCCTACACTTAACCGAAGCCATTCCCT
GGTTCACCTCAACCCAAGGACTCTGCCCTGCCAAAGAACTGGTGAGGGGAGGGAGAGAGAACCACCGTTCATTCCTTGCCTCTTGCTCC
CAGGTGATAGTCCCTTCACATCAGTATCTCCTATGCTTCTGAAAAAAACAGACGAACAGCATTACCACTGCTAAGTTGATCCTGGTTTT
CCAAACAACGACATACAAAGGTTCAGAGGGTTGCAGAGCTTAATGGGTACGAACACAACAAAAATAACCCAGGGCCCCTGTCTTGAAAA
CAAGCCTTGCTGCCTTGCTTAGTTGGGTGTCCTTCCGCTGGTTAGGGGTCTGAGAATGAGCGCTACAGGCTCCATTAGGAGCTTTGACA
GAGTGCCACGAAAGAGGTACTATGATCTCTTTATACCTAGTACAGGTACAAACAGACATGAAATAACCCATCCATGCACCCAGGGGATT
CAAAACCACTTTATCCTTAGTGCATCCAGGAGGAAGATCCTAGCTACGAAGGAGAGGGCAGGAGTCATTAAAGCTTCACTACTCAAGAA
CTGGTAGACTTCAGTTCCCCTCCTTGTTTGTGGATTGGGGGGTTGGGGTGGAGGGTGTTGTGTGTACTCAGAGGCACATAGCTCACTCC
TACCTTACCCACTTAGTTTTTAACATCAGATCCCTGCTTGCTCCTGGGAAGAAGCCAGTTTAGAAGTGCTACTGGTCACATCATAAAAT
AAAACCTTGTTTTACATGAGTCATTATTTTAGAAATTGCAAGCTCGCCTTCCTCCCAAGCACTTTCACACACTCATGACACACGCTCAT
CAATGGCCCATGAGAAAGCCTTTTGGAACAGGTATCTTATTAATTATAAGCCTCTGAAAAGCCACAATCCAAACCAGAAACTGAAACAT
GTACGTAAAGTCCAGGGAGAAAGGTACCCTAAATGTCCTAAATGACCCCATTGTCTCAGAGAACGTCTCTAGCATCCTGGAGTGTCTTT
GGGACTTTAATTCACCATCATTCATAAACCAATAAGTAATTACTATCTTTGACACCCCCCCCCCATGATCTATTGAAGCGTTTAACATT
TAGTTTTTGATCTTATTTTTGGCTCTTTTAGACTCACCCTCACCTTCAAACTGTTACACCCTCTATCCTACATTCAAAAGAAAACACAA
ACCCCAGTAGTAGTTTGAAATACTTTGAGTAGTTCAAAGGATTAGCAAAAGGGGGCAGCAGGGGGTGCTCTTAAATAGTTCCCTCTTTT
TCCCCTTTGTGAGCCTAGGCTGCACTGCACCCCTGGGGTGACTCACTTGAGACCTGGGAAGGTGTTAGGTTGAATCACTCAGTCCAGGC
AAGCCCAAAGAATAGAGGAAAGCATTCCTCATTAGGAAAACAACTCCTGTTCCAAATGATCAGGAAACAAGTTTAGAGATTCAGATTTG
GCTGTGGGGATGGAATCGAAGTATCACAGCTCCCTATCTGGGCACTTCTCAGCTTTACCAAGCCAGGGAATGGTCTGAAAACAGGACAT
CGGCCAGCTTCCTTCCAGAAAGTCAGGCTGATCTTGACCATAACACACGAAGGCTCTCCCAGCCAATCTGGAGTCCCAGGGGTCTGCAA
CATTCTGCATCAATTTATAGATAACAATCACGATGAGGGGTGAAGTGGGAGGAGCTTTCAGCTTGCAATCTGGGATATACAAGAATTAG
CTAGTCTCTTCTGTTGTTCACCCATGACACTCCAACTCAGTTTCCCCGAGAGGATCCCTGGGATGGTGCTCTTCAGGATAAACTGAGTG
AGGAGGAAGTGTGACTTTATGCTATCATTCGGGGACAACACTAAAAAGCAATCAATTACACTTTAAGTTGAACAAAAGTTTCACAATAC
CAGGATAGTCCTTCAGTCATCCAAAGCTTGAACACGTATGTAGAGTCTTCTGCTGTTCTTTAAGATGGACTTTGGCACTAACCTTGGAA
AAGAGGGCCAAAACCCACCCCCCAAAAACAAACAGAACAAAAAACGTGAAAGAAGATTTTCTGTGGTTTGTTTACAATAAGAGATGAGT
CACAATAATGATTTTTTTTTTTTTTTTTAGCATGACTGCTAAGGAACCCTGAAGCATTTCTTCCCAACAAAAACTAATCTTTAGCTTAT
AACATGCTGGAAGATGAGCTTCAAGCCTCTCTATAAATACATCACTGGGTATCTTCAGGGAACAAACTCTTCACTTAAGGTTAGAGACC
ATCCTGGATCGATTTCAAGAACAGAGGTTTATTTTAAACAATGAAGCCTCGGCTACAGATCTCGTTCAGTGATAAGACACTTGTCTAGC
ATGTATAAGCACCCATGCTTGACACCACAGGAATAAATAAAACCAAAAATGAGGTTCCTACTCATTCCCCAAACTTTAGCAATTTTGGT
GGTGGTGGTGGTGGTGGTTCCACGTCTTTCCACAGAAATCCAACAAACTGCCAATTTAGCCAAAACATCTAGCTCTAGCGACTTTGCCA
AACTGGTGTGAGTTGACTCAATGACTGAGTGGATTTCCTTTTTGGGTTTTACTTCACTCAATATTGGTCTACACTAAGCTCTTTAGGAA
GACAGGGTTGAGAGAGGAACAAGTGTTAATGGGATGTAGATCCTCTGAGGCTGAAAGGAATGTCCTTTGTCTCTAAACAATGCCCAGCC
GCTCAGGAACACTCTCTTCCAGTCTTCACCCGACCATCTCATTAAAGGCTAAACTCTCCAGTTCGGGTCTATATGGCTAGGAAAGAGGA
GCTTTGGGGAAAGCTCTGTCAGAATATACCTGCTTTCTGCAGGGGGCGGGACGACCCCCACAAACAACTCAGCTGGAGAACAATTGTTA
CATGTGACAATACTGAAGTTTGTACACAGACATGTGAAATTGCTGGCCTGAAGTCACATAACCACTAACTAACGATAATAGCAATTAAT
GTTTACTGTGTGCTAAAAGTTTCACATACATTATCTCCTTTTATTCCCAAAGCAACCCCCTATACACACACACACCTCCTTTTTGGTTT
TGGTTTTACTTTTTAAATTTTACTATTAAATTTTAAATATGTTTTTTTTTTAAATTTACTCCACTAGAGAGGGAACCTAGAACCTCCTG
CATCCAAGGTAACTGATCAATGGGTGAGTCACATCTCTATAGAGATTGTAGGTTGTTTCACCAAAAGTATTAGGGCATTTCCTACAGAG
TGGTGGAAGTTCGAGTCCTGGGAACATGGAATAACCATGGAATCCAGTAAGTCTTGAGAAAGAAGCTAAAGGAGAAAATGTCAGCACCA
GATGGCACGGAAGCAAAGAAGAAGGAGAGCTAAAACCAACAAGCTAAAACCAAAAACGCTTGCTATTTCAACTGTGCCTAACGTACCAG
GAAGTACAGCAAGGATCCACTGGCTTAGTGAGGGAATGCTTCTCTAGGCCTCTCCTATCCACTTCCCCTTCCACCCAGTATCCTCCTAC
TCCTTCATCTGCTAGAGACAACTGCCCATAGGTAATTAATTAGCTGTCCCAGCACCCAACTCTCCTGCAGTACTTTGGTTCTGTTTATT
TGTTTTGGCAATAGCATCTCATTAGGCACGCCATACTCGCCTCAAACTCTGGAATTTCTTCCCTCAGCCTCTTAAGTGTTGTCTTGGGA
TTACCAGTTTGGGACACTTATTCTCCACCTTTCATGCAGGGCTTTATTCACCAATTTCCACTGTGTGATTCTACGATGCAAATACTAGT
GCTTTTTCAAAACAGGGTTTCTCTGTGTCTCAGAACCATTGTCTTTTCTACCACTCCAAACCCAACCATCTTTTCAAACCACTGGTAAG
CCAAGTCCTTTCTGGATTAAGTCTGAAGCCATTCTCCACTCCTATTCGATACACTTACTACCTACCACAAACACGCCACCCTCCCACCC
TCAATGCCAGTTTTGGGCTTCTCCTTCTATCCTTGCACACCAGTTGTCTTCATCATCAGATCAACCCCTTGAAGAATCGAAGTTCATTG
CTCTTGCTTAATCATGCCTGACAAGAAAGTCCAATTCACTAATTCTGTAAACATCTAAGTCAAGCGCTCTTTGATGGGCTCAATGGTGC
ATCAGACCTGAGTAACGGGTGGAGTACAAGCAAAGTTGTTTCAAGTTGCATAGCGAGAAAGGTCAAGGAAAGAGATGGGGGTTCATCAG
GTGAAGGGAAGGATTTTCATTACACTTCCTTTGCTTCAAATTTGGTTTTTGAGCTGAACACACGAAGCCTAATTCATTTCCATGATAGT
AGGCTAGACTTGAGAGGGAAGAACTGCTGACAGAAAAGCAGGCGAATTGTACTGACTGGTTTTGTGTGTAATGTGTGTGTGTGTGTGTG
TGTGTGTGTGTGTGTCTCTGTGTGTGTGTGTGAACTTGACACAAGCTGAAAGTTATCACAGAGCCTCACTTGGGGAAAGCCTCCGTGAG
ATCCAGCTGTAAAGCATTTTCTCAATTAGTGGTGAAGGAGGGCCTATGTGGCTGGTGCCATCCCTCGCCTGGTAGTCTTGAGTTCTCTA
AGAGAGCAAGCTGAGCAAGCCACGGGAACCAAGCCACTAAGTAACATCCTTCCAAGACCTCTGCATCAGCTCCTGCTTCCTCACCTGCT
TGAGTTCCAGTCCTGACTTCCTTTGGTAATGAACAGCAGTGTGTAAGTGGACAGTGAATAATCCTTTTCTTTCCCAACTTGCTTTTTGG
ACATGATGTTTTGTGCAGGATATAAACCCTGACTAAGACAACAATCAAAGTGTCTAGTCAAACTGAGTTACTCGACCGGTAGGTAGCTT
TGTTTTGCTTGCTTGATGTCTGGACGCCTCTCTTAGCTGGGTATGGCTGAACAAATGAAAAAAGAATATGTTCCCCCTACTTAGGTAAG
TATTCTTCCAGGTGGTGATGTCACTGGCCAAGCTGTTCTCCCTTGGCTTCCCACAGCTCTCTTCCTTTACCACATACCATGTCCATCTT
TACACAAATGACTCTAAACCTCTTCTCTCCACAGACACCTTCAATCCACATGAGATAAAGCCTCCACATTTTGTAGCTCTGGCTGACCT
GGAACTCACTATGCACACCAGCCTTGCATTGAACTTTCAGAGATCTGCCTATATCTTTCTTAGGAGTGCTAGGACTAAACGCATGCACC
ACATTTAGTCCTGCThAACTTCTTTTTTTGTTTTTTTGGTTTTTTCGAGACAGCGTTTCTCTGTGTACCCCTGGCTGTCCTGGAACTCA
CTCTGTAGACCAGGCTGGCCTCAAACTTAGAAATCCACCTGTCTCTGCCTCCCAAGTGCTGAGATTAAAGATGTCTGGTACTACTGCTT
AGACTGACACACAAAATATTGTTGAAGCCAGTGAGATAAAGCTCACCTTCAATCTCACACGTAGGCCAGGCAGACTTTTGTGAGTTCTG
GGCCAGCCAGAGCTTCATATGAAAAGTAATAATAATGATAATAATAATAATAATTTTAATAAACAAATGGGTGTATGAAAGAGTAAATC
TGCACATATTTCTGGCATCTGTTTTAAQATTCTACTTCCTTAGGCTGTCAGTCATTCTCACCCCATTCAACCCTTTCCTCTGCACTGTG
ATTGACAAATCTTCTCTCCCTCTGGTGACTACCTAATTCATCCCACACAGCATCAGCCTTGGCAACAACTTCTCTATGCAGGGACTGTA
AGTTCCAGACAACAGAAACTCTGTTCCAATTCACAAAATCAACTAGGAAAGGAGAAGAGACAGAGGACAGAGGAAAGGAAGGAAGAAGG
GAGAAAAGGAGGGAGGGAGGGAGGGAGGGAGGGAAAATGTCAATTTATGCAACTCTTTCATGATTCATCCAAGGATCCTCTCTGATTCT
TTTTTGGTTGTTGTTGTTTTTGTTTTTTCGAGACAGCGTTTCTCTGTATAGCCCTGACTGTCCTGGAACTCACTTTGTAGACCAGGCTG
GCCTCGAACTCAGAAACCCACCTGCCTCTGCCTCCCAAGTGCTGGGATTAAAGATGTGTGCCACCACGCCCAGCTAAACCTCTTCCTTC
TAGCCAGAAGAACACAGCAGAAAGAACTATGATCAGTCTTCTGGAACCATGTGCTTACCCTTAAACCAGACAATTCACTTGGTTCCCAG
GACCACACTCCTCCTGCAAAAGTTGGAACCCTGTGATTCTCAGCTTCTGAGGCAAAGGGAAGACCTATCCAAGATAAAAAACCAACAAC
AACAAAAAAGTCAATATAAGAAAAGAAAAAAATGTTCTCTAGATACATTCTGGGTAGTGATGGTTCTCACTGTTCAGAGATATAAGAAA
AGAGCAGATGCTTAAAACCCTGACTCTTGAGAACAACTTTTGGTAAGAGAGACTCCTTTTTTTAAAATTTTATTTATTTATTTATTTAC
TTACTTATTATATGGTAAGTACACTGGTAGCTGTCTTCAGACACTCCAGAAGAGAGTATCAGATCTCATTACAGATGGTTGTGACCCAC
CATGTGGTTGCTGGGATTTGAACTCAGGATCTTTGGAAGAACAGTCAGTGCTCTTAACCACTGAGCTATCTTACCCAGCCCCCCACCCA
CCCCCCCAACCCCGTTAAGAGAGATTCGAATGGTCCATCTTAGAGGTATTGAAATTGAGTCTGAAGTGGGAGGCCTAGGGGCTGTTTTG
AGCCAACTCAGCCCAGGTGATTTGCAGTGAGGTGGGAATTCCAAGGGCAAACAAGGATCAGCAAAAGCTATACCCCTCTACCACAGATT
TGGAGAGAATTCAGATGGGAGGCCTGTTCCAGCCATTGCTCAGTAAGTAACCAACAGTCTAGCTACCTCTTCATCTCCCTCCTTCTACC
ACACCATTAAGTCCCTCCACCCCATCAGCCTCAATGATTCTTTCTTTTTTTTTTTTTAATTAGGTATTTTCCTCATTTACATTTCCAAT
GCTATCCCAAAAGTCCCCCATACCCTCCCCCTCCACTCCAACTTTTTGGCCCTGTTGTTCCCCGCCTCAATGCTTCTTCGCTCTATCCA
TGTCTCCTTGCCCACTTTGCTTCTGCCAACCGATATTCTTTACCTGGGACTCTCTAGCAGATGCCAACTGCCTGTCTCTCATCTGGTTT
CCTCAAACCTACAAAAATGCTCTCCCAAATAAGCCCGATCAGTTTACTCCCTGGCGTAGGAGAACTTCATGATTCATCTGCTCTGCTTC
TAAAATACAGATCTGTGGGCCTGGTAGTGCAGATGTTAATCCCAACTACTCTACTTCCCAGCCCTAGGGAATGGACTCCATGTTCAAGG
TCTGCCTGGACAGCAAGATGAGTGCTGGGGAAGCCACAGAAAATGAGTAAAACTTTCTGAAAAGTAAAGCATTATAAAAACAAACAAAC
AAAGGTCCAGACAACTGGGGAGATGATTGGCTGGTTAAGAGTACTTGCTCTTCTCCATGGGACCCAAGTTCAGTTCCTAGAAGCCGAGT
AGCTTGCACCTTCAATTATGGCCCCCTCTTCTGTTTTCTCAGGACCAGGTCCACACAGTGCATATAACATACATGTAAGCAAAATACTC
ACACACATAAAATTAAAAATAAATAAATCTTTCAGGCAAAACTGACCTGGGATATAGCTCAAGGATAGACGATTTGCATGGCATGCTAG
CAGCCCTGACTTCCATCTACAGTCCAGAAAGACAAGTAAATCAGAAATGCCTTTGACCCACTGAAGAGATGATTCAGAGGTTAAGAGCA
CTGGCTGCTCTTCCAGAAGACCTGGGTTCAATTCCTACTACCTGCATGAGAGCTGACTCCAGTTCCAGGGAATCTGACCCTATCACTCA
GACACTGTTCTTGTCATCTGCACACGCGATGATCACACACATATCATTTCACATGTGGGGATACTGACAATCAGAGTCAGCAGGTAACT
GTCTCAACTTCAACTAAGCAGGGCAGTTTGTTAGCAGGGATGGAATCTCGATTCTTATTGCCCCCCAGCTTCTTCACTTCCTGCTGCCT
CTCTACAACCACACCATATTGAAATTACCTTCTTCCCTCTCTTGCTTCCGCACTGAAACTGGGGCTGAATCTAGCTGGCTACCTGACCT
GATTCTCTCCTATCAGATGGATGACTTGCTGTCCTCCTGGGTCATCTCCCGTGTTTAGCAAGACAGGGTATAGAGTTTTAAATGAGTGT
AGGCAGAGTAACATGAATCATCACACTTTTACCCAGAGTGCATGAATCATCAGACTTTCCTACGCCAGGGAGTAACTGAGCACGCATAT
TTTGGAGACCACTTCTCTACTCCTAGGGTCAGAAGAGCCTGCAGATGATCTACTCAAAGCAATTTTATCTGCCATTCTGTTCAGTAGTG
AGGCAAGGATCATGTTACCTGCAACGGGGCCAATCTTTGCCTCTGTTTTTGAAGTCTCTGGAGAAGTAGCCGTCTTCCTGCGCTGGGGA
TATGATTAAGCAGTAGAACATCCACATAATTTCATGAGGCTAACATACAGCTCTTCAGAGATCAGCCTACTTATTTATGACACAATTGT
TTTAAGACATCAGGTATTCTAGGAGATAAGATTCCTTCTCTTGCTTTCCTGGTACCCAGAATGATCTCCTAAACCACAAGTAATTGGCA
CAGAGATGGATCTTCCAAACTGACCCACATCTACACAGATCTTATCTTCATACCCAAGAACTCCCCAGTCAATGGATATTTGGGGCATT
TCCTATCCACACACACGTCAGTCTGGAATAACCGTACTCCAGCACTTAGTAGAAAGGATGTATACTGAACCAGTATTTACAAATAATTA
ACCAGATTATTTTCAGGGCACAAATTAGTGGAGCAAGGATTGCAGAGTTACATCAACATCTGATAGCATTGCGGTTCAGCGAAAGTTCC
CAGCCAGGGTACAGACGATGGTGATGAAGAAGCATGCTCAATATCTTCAGGCGCTGCTATGAGAAAAQAATGAAACTCTCTAGGGGAAC
TGAAAGCATACAAGGATGTAAAGAATAGGAAGACCAAAAGGACTTCAACCCTTGGATGAGACACAGGGTGATCCAATACCCGGTGACCT
GCAGGGACTCACGCCCACTGGTTATGATCGCAACCTACCCACAGTCGTGAGTATGATGCAGTGGGAACTCACAGTACTGAAAACCAACA
CACTTAACTTGCCTGTGCTGCTGTGAACGTCCATTCAATGAGGGTCACCAAGCTCAAATAAGATAATCGCAAATACTGAGTGGTTCTGA
CACGAATCCAACCATCAAAAAAAAACTCCTGACACTTGCAGAATGTTTATGTTTCTAATGCTGGGCTAAGTACTTCACCTGGATTATTT
CACCAATTCCTCCTCCTCCTCTCCCTGTTCTCCCTTCTCCTCCTCTTCTTCCTGTTCTTCCTCCTCCTCTTCCTCTTCTTCCAAAGATT
CATGTGTATCAAGGTATTCTCAA
MOUSE SEQUENCE - mRna

(SEQ ID NO: 20)

GATTGGGGTACGCCCTGCGCCACGTTTCCGCACCAACCAGAGCTGGATAACTCTAGACTTGCTTCCCTTGCTGTGCCCCCTCCACCACA
CAGCCACGACGATGCCCCTCAACGTGAACTTCACCAACAGGAACTATGACCTCGACTACGACTCCGTACAGCCCTATTTCATCTGCGAC
GAGCAACAGAATTTCTATCACCACCAACAGCACACCGAGCTGCAGCCGCCCGCGCCCACTCACCATATCTCGAAGAGATTCCACCTCCT
TCCCACCCCGCCCCTGTCCCCCACCCGCCGCTCCGGGCTCTCCTCTCCATCCTATGTTGCGGTCGCTACCTCCTTCTCCCCAACGCAAG
ACCATGACGCCGGCCGTCCCAACTTCTCCACCGCCGATCAGCTGGACATGATCACCGACTTACTTGGAGGAGACATGGTGAACCAGAGC
TTCATCTGCGATCCTGACGACGAGACCTTCATCAAGAACATCATCATCCACGACTCTATCTGCAGCGGTTTCTCAGCCGCTGCCAAGCT
GGTCTCGGAGAAGCTCGCCTCCTACCACGCTGCGCGCAAAGACAGCACCAGCCTGAGCCCCGCCCGCGGGCACAGCGTCTGCTCCACCT
CCACCCTGTACCTGCAGCACCTCACCGCCGCCGCGTCCGAGTCCATTCACCCCTCAGTCCTCTTTCCCTACCCGCTCAACGACACCACC
TCGCCCAAATCCTGTACCTCGTCCGATTCCACGGCCTTCTCTCCTTCCTCCGACTCGCTGCTGTCCTCCGAGTCCTCCCCACGGGCCAG
CCCTGAGCCCCTAGTCCTCCATGACGAGACACCGCCCACCACCAGCAGCGACTCTGAAGAAGACCAAGAAGATCACAAACAAATTGATG
TGCTGTCTOTGGAGAAGAGGCAAACCCCTSCCAAGAGGTCGGAGTCCGOCTCATCTCCATCCCGAGGCCACAGCAAACCTCCGCACAGC
CCACTGGTCCTCAAGAGGTGCCACGTCTCCACTCACCAGCACAACTACCCGCACCCCCCTCCACAAGGAAGGACTATCCAGCTGCCAAA
GAGGGCCAAGTTGGACAGTCGCAGGGTCCTGAAGCAGATCAGCAACAACCGCAAGTGCTCCAGCCCCAGGTCCTCAGACACGGAGGAAA
ACGACAAGAGGCGGACACACAACGTCTTGGAACGTCAGAGGAGGAACGAGCTGAAGCGCAGCTTTTTTGCCCTGCGTGACCAGATCCCT
GAATTGGAAAACAACGAAAAGGCCCCCAAGGTAGTGATCCTCAAAAAAGCCACCGCCTACATCCTGTCCATTCAAGCAGACGAGCACAA
GCTCACCTCTGAAAAGGACTTATTGAGGAAACGACGAGAACAGTTGAAACACAAACTCGAACAGCTTCGAAACTCTGGTGCATAAACTG
ACCTAACTCGAGGAGGAGCTGGAATCTCTCGTGAGAGTAAGGAGAACGGTTCCTTCTGACAGAACTGATGCGCTGGAATTAAAATGCAT
GCTCAAAGCCTAACCTCAACAACCTTGGCTGGGGCTTTGGGACTGTAAGCTTCAGCCATAATTTTAACTGCCTCAAACTTAAATAGATA
AAAGAACTTTTTTTTATGCTTCCCATCTTTTTTCTTTTTCCTTTTAACAGATTTGTATTTAATTGTTTTTTTAAAAAAATCTTAAAATC
TATCCAATTTTCCCATGTAAATAGGGCCTTGAAATGTAAATAACTTTAATAAAACGTTTATAACAGTTAAAAA
MOUSE SEQUENCE - CODING

(SEQ ID NO: 21)

ATGCCCCTCAACGTGAACTTCACCAACAGGAACTATGACCTCGACTACGACTCCGTACAGCCCTATTTCATCTGCGACGAGGAAGAGAA
TTTCTATCACCAGCAACAGCAGAGCGAGCTGCAGCCGCCCGCGCCCAGTGAGGATATCTGGAAGAAATTCGAGCTGCTTCCCACCCCGC
CCCTGTCCCCGAGCCGCCGCTCCGGGCTCTGCTCTCCATCCTATGTTGCGGTCGCTACGTCCTTCTCCCCAAGGGAAGACGATGACGGC
GGCGGTGGCAACTTCTCCACCGCCGATCAGCTGGAGATGATGACCGAGTTACTTGGAGGAGACATGGTGAACCAGAGCTTCATCTGCGA
TCCTGACGACGAGACCTTCATCAAGAACATCATCATCCAGGACTGTATGTGGAGCGGTTTCTCAGCCGCTGCCAAGCTGGTCTCGGAGA
AGCTGGCCTCCTACCAGGCTGCGCGCAAAGACAGCACCAGCCTGAGCCCCGCCCGCGGGCACAGCGTCTGCTCCACCTCCAGCCTGTAC
CTGCAGGACCTCACCGCCGCCGCGTCCGAGTGCATTGACCCCTCAGTGGTCTTTCCCTACCCGCTCAACGACAGCAGCTCGCCCAAATC
CTGTACCTCGTCCGATTCCACGGCCTTCTCTCCTTCCTCGGACTCGCTGCTGTCCTCCGAGTCCTCCCCACGGGCCAGCCCTGAGCCCC
TAGTGCTGCATGAGGAGACACCGCCCACCACCAGCAGCGACTCTGAAGAAGAGCAAGAAGATGAGGAAGAAATTGATGTGGTGTCTGTG
GAGAAGAGGCAAACCCCTGCCAAGAGGTCGCAGTCGGGCTCATCTCCATCCCGAGGCCACAGCAAACCTCCGCACAGCCCACTGGTCCT
CAAGAGGTGCCACGTCTCCACTCACCAGCACAACTACGCCGCACCCCCCTCCACAAGGAAGGACTATCCAGCTGCCAAGAGGGCCAAGT
TGGACAGTGGCAGGGTCCTGAAGCAGATCAGCAACAACCGCAAGTGCTCCAGCCCCAGGTCCTCAGACACGGAGGAAAACGACAAGAGG
CGGACACACAACGTCTTGGAACGTCAGAGGAGGAACGAGCTGAAGCGCAGCTTTTTTGCCCTGCGTGACCAGATCCCTGAATTGGAAAA
CAACGAAAAGGCCCCCAAGGTAGTGATCCTCAAAAAAGCCACCGCCTACATCCTGTCCATTCAAGCAGACGAGCACAAGCTCACCTCTG
AAAAGGACTTATTGAGGAAACGACGAGAACAGTTGAAACACAAACTCGAACAGCTTCGAAACTCTGGTGCATAA
HUMAN SEQUENCE - GENOMIC

(SEQ ID NO: 22)

ACTCCCACCACACCTATTCAACATAGTATTGGATTCCCTGGCCAGAACAATTAGGCATGAGAGAGAAACAACAGGCATCCAAACAAGAA
GAGAGAAGGTCAAATTATCCCTGTTTGCAGACAATATGATTCTGCATCTAGAAAACTCCATAGCCTCTGCCCCGAAACTCCTTGAGTTG
ATAATCAACTTCAGCAAAGTTTCAGGATACAAGATCAATGTACAAAAATTAGTAGCACTCCTATACACTAACATCACCCAAGCCAAGAG
GCAAATCAGGAACACAATCCCATTCACAATTGCCACAAAATAATAAAAAGCTTAGGAATACAGTTAACTAGCGAGGTGAAAGATCTCTA
CTATGAGAATTAAAAACACTCTTCAAAGAAATCAGAGATGACCCAAAACAATTGGAAGAACATTCCATGTTCATGCAAGGGAAGAATCA
ATATTGGTAAACTGGCCATACACCTCAAAACAATTTACAGATTCAATTTTATTCCTATCAAACTACCAATGACATTCTACACAGAAATG
GAAATAACTACTCAAAAATTCATATGGAGCTAATAAAAGAGTCTAAATACACAAGGCAATCCTAGGCAAAAAGAGCAAAGCTGAAGGAA
TCACATTGACCAACTTCAAATTGTACTACAAGGATGTAGTAATCAAAACAGCATGGAACTGATACCAAAACAGACACATAGACCAATGG
AACAAAATAGTGAGCACAGAAATAAAGCCACATACCTACAACCATTGGATCTTTGACAAATCTGACAGAAACAAGCAAGAGGGAAAAGA
CTCCCTATTAAATTAATGGTGCTGTGATTACTGGTTAGCCATATGAAGAAAACTGAAACTAGACTTATGCCATATATAAAATCCATATG
CCGGAAAGCTGAGGCAGGAGAATCACTTGAACCCGGGAGGTGGAGGTTGCAGTGAGCCGAGATCACACTACTGCACTGCAGCCTAGCGA
CAGAGTGAGACTCCGTTGCAAAAATAAATAAATAAATAAATAAATAAATAAATAAATAAATATCAACTCAAGGTGGATTAAAGACCAAA
ATGTAAAACTCTAAAACTATAAAAACCCTGGAAGATACCCTAGGAAATACCATTTTGGACATAGGAGCTGACAACGATTTTTTGAAAAA
AACACCAAAAGCAAAAGTTGACAAATAAGACCTAATTAAACTAAAGAACTTCTGCACAGCAAAAGAAACTATCAACAGAGTAAATAGGC
AACCTGCAGAATGGAGGAAAATATTGACAAACTATATATGTGACAAAGGCCTAATATACAAAATCTACGAGGAACTTAAACATACAAGG
AAAAATCAAACAACCCACCTCACTAAAAAGTGGGAAAAGGACATAAACCGACACGTTTCAAAAGAAGACATACAAGCATATACATACAA
GCCAACAAGCATATGAAAAAATGCTCATCATGACTAATCATTAGAGAAATGCAAATCAAAGCAGCAATAAGATACCATCTCACACTAGT
CAGAATAGCTATTATTAAAAAGTTAAAAAATCACAGATGCTGGTGAGGTTGTGGACAAAAGGGAACGCTTATACACTGCTGGTGGGAGT
GTAAATTAGTTCAACCATTGTGGAAAGCAGTGTGGTGATTCCTCAAGTAATTAAAAACAGAACTACCATTTGACCCAGCAATCCCACTA
CTGGATATAAACCCAAATAAATATAAATCTTTCCACCACGAAAACACATGCAACCATGTGTTCATTGTAGCTCTATTCACAATAGCAAA
GGCATGAAATCAGCCTAAATGCTCAACTACTGCAGACTGGATAAAGAAAATGTGGTACATACACACGATGCAATACTAAGCATCCATTA
AAAAAAAAAAAGAAATCATGTCCTTTTCAGGAACATGAATGGAATCAGAGGCCATTATCCTTAGCAAAACTACCACAGGAACAGAAACC
AAACCTATGTTTTCACTTATAATTGGGAGCTAAATAATGATAACCCATGGGCAGAAAGAGGCCTCACACTGTGAGGCCTGCGTGAAGGA
GGAGGATGGGAGGAGAAAGAAGTCCAGGGGAAAAAAACTTCGGGTACTGTGCTTAGTACCCAGACAGCAAAATAATCTCTACACATTGA
TGGGACGTATCTCAAAATAATAAGAGCTATCTATGACAAACACACAGCCAATATCATACTGAATGGGCAAAAACTGGAAGCATTCCCTT
TGAAAACTGGCACAAGACAAGGATGCCTTCTCTCACCACTCCTATTCAACATAGTGTTGGAAGTTCTGGCCAGGGCAATCAGGCAGGAG
AAAGAAATAAAGGGTATTCAATTAGGAAAAGAGGAAGTCAAATTGTCCTTGTTTGCAGATGACATGATTGTATATTTAGAAAACCCCAT
CGTCTCAGCCCCAAATATCCTTAAGCTGATAAGCAACTTCAGCAAAGTCTCAGGATACAAAATCAATGTGCAAAAATCGCAAGCATTCT
TATACAACAATAACAGACAGAGAGCCAAATCCTGAGTGAACTCCCATTCACAATTGCTTCAAAGACAATAAAATACCTAGGAATCCAAC
TTACAAGGGATGTGAAGGACCTCTTCAAGGAGAACTACAAAACACTGTTCAATGAAATAAAAGAGGACACAAACAAATGGAAGAACATT
CCATGCTCGTGAATAGGAAGAATCAATATCGTGAAAACGCCCATATTGCCCAAGGTAATTTATAGATTCAATGCCATCCCCATCAAGCT
ACCAATGACTTTCTTCACGAATTGGAAAATAACTACTTTAAAGTTCATATGGAACCAAAAGAGAGCCCGCATTGCCAAGTCAATCCTAA
GCCAAAAGAACAAACCTGGACGCATCACGCTACCTCACTTCAAACTATACTACAAGGCTACAGTAACCAAAACAGCATGCTACTGGTAC
CAAAACAGAQATATAGACCAAAGGAAGACAACAGACCCCTCACAAATAATGCTGCATATCTACAACCATCTTACCTTTGACAAACCTGA
CAAAACCAAGAAATGGGGAAAGGATTCCCTATTTAATAAATGGTGTTGGAAAACTGGCTAGCCATATGTACAAAGCTGAAACTGCCTCC
CTTCCTTACACCTTATACAAAAATTAATTCAAGATGGATTAAAGACTTAAATGTTAGACCTCAAACCATAAAAACCCTAGAAGAAAACC
TAGGCAATACCACTCAGGACATAGGCATGAGCAAGGACTTCATGTCAAAAACACCAAAAGCAATGGCGACAAAAGCCAAAATTGACAAA
TGGGATCTAATTAAACTAAACACCTTCTGCACAGCAAAAGAAACTACCATCACACTCAACAGGCAACCTAAAGAATGCCACAAAATTTT
TGCAATCTACTCATCTCACAAACCCCTAATATCCAGAATCTACAATGAACTCAAACAAATTTACAACAAAAAAACAAACAACCCCATCA
AAAAGTGGGTGAAGGATATGAACAGACACTTCTCAGAAGAAGATATTTATGCAGCCAAAAGACACATCAAAAAATGCTCATCATCACTG
CCCATCACAGAAATGCAAATCAAAACCACAATGACATACCATTTCACACCACTTACAATGGTGATCATTAAAATGTCAGAAAACAACAC
GTGCTGCAGAGGATGTGGAGAAACAGGAACACTTTTACCCTCTTCCTGGCACTGCAAACAAGTTCAACCATTGTGGAAGTCAGTGTGGC
GATTCCTCAGGGATCTAGAACTAGAAATACCATTTGACCCACCCATCCCATTACTGGGTATATACCCAAAAGATTCTAAGTCATCCTGC
TATAAAGACATGTGCGCACCTATGTTTATTCCGGCACTATTCACAATAGCAAACACTTGGAACCAACTGAAATGTCCATCAATGATAGA
CTGGATTAAGAAAATGTQGCACATATACACCATGGAATACTATGCAGCCATAAAAAATGATGACTTCATGTCCTTTGTACCGACATGGA
TGAAGCTGGAAACCATCATTCTCAGCACACTATCTCAACGACAAAAAACCATACACCACATGTTCTCACTCATAGGTGAGAATTAAACA
ATCAGAACAGATGGACACAGGAACCGAAACATCACACACCGCCCCCTCTTGTGGGGTAGGGGCAGGGGGGATGCATACGATTAGGAGAT
ATATCTAATATTAAATGACCAATTAATGAGTGCGGCACACCAACATGGCACATGTATACATATGTAACAAACCTGCACATTCTGCACAT
ATACCCTAAAACTTAAAGTATAATAAAAAAAATCTGTACACTAAAACTCCAACTCATGAGTTTACCCATATAAAAAACCTGAACCTAAA
ATAAAAGTTAAAATATTCAAATATAAATAAGTAAATAAATAAAATGACAAACCCATGGCTAACATCATGTTGAATAACGAAAAGTTGAA
TGCTTTTTTTTCTAAAATCCAGGAGAAGACAAGGATGTCCATTCTCTCTTCTACTCAACATGGTACTGGGAGTCTTAGGTAGAACAATT
ACACAAGAGAATGAAATAAACGTCATTCAAATTGGACAGGAGGAAGTCCTTTTCTGCAGTAACATCATCTTATACCTATAAAACCCTAT
AGACTCAACCAAAGAAAAATAACTGCTAGTATTGATAAATGAATTCAGTAAAACTTCAAGTTACAAATTTAATGTACAATAATTAGTAG
TGTTTCTACACAGCAACAGCAAACTATCTGAAAAAGAAATCAACAAATCTATCTCTTTTACAATAGCTACAAAAATTTCAAAAAATTTC
AAAAAATACCTAGAAATAAATTTAACCAAGGAGGTGAAAGATCTCTACAGTAAAAAATAATAAAATATGGATGAAAGAAATTGAAGAGC
ATACTAATAAATGTAAAGTTAACCCATGCTCATAGATTTGTAGAGTTAATCTTGTTAAAATGTCCATACTACCCAAACCAATGTACAGA
TACAATGCAATCTCTATCAAAATACTAAGGACTTTCCTCACAGAAGTAAAATATATATATTTTTTAATATATAAAATGTATATATAAAT
ATAATATATAATAATGAATATTATATATGATTATAATATTATATGATTATATACTATTAATATTATTAATAATTATATTATTATATATA
TTATATATATTATTATATATTGTATATAAATATATATTATATACTATATTATTATATATTGTATATAAATATATATTATATATACTATA
TATTTATATACTATATATATAGTGTATATATATATTTATATAGTATATATAATATATACACTATATATTTATATAGTATGTATATATAT
ATACTATATATAATACATAAAATATATATACATATATAGTATATATATGAAATATATATATACTAAATTTGCACAGAACCACATGATTC
CAAACAGCCAAAGCAATCTGGAGCACAAAGAACAAAGCTAGATGCATTACACTACCTGGCTTTTAAATTTTCTACAAAGCAATAGTAAA
CAAAATAGCATAGTACTAACATGAAAACAGATCCATAGACTGATGGAGGAGAATAGAGACCCCAAAAATGAATCCATACAATTATAGCC
AACTAGTTTTTGACATATGTACCAAGAAAACACATTCGGCAATGGAAAGTCTCTGCAATAAATGGTGCTGGATAAACTGAATATCCACA
AGCAGAAGAAAGAAACTAGGTGCTTCTCTCTCCCAACATATAAAAATCAACTCAAAATGGATTAAAGACTTGAATATAAAACATGAAAC
TAAGAACATCCTTGAAGAAAACACAGGGCAAACAATTCATGATATTGGTCTGGGAAAGAATTTTTCAAATAAGACCTCAAAAGCACAAA
CAACCAAAGCTAAAATAGATGAATGGGATAATATCAAGCCAAAAAGCTTTTACATCTTAAAGCAAACAACAGAATCAAGAAATGACCTA
CAGAATGAAGAAATATTCACATACTATGCATCTGACAAGAGGTTAATATGAAACTATGTAATCAATGCAAGCAACTCAATAGGAAACAC
ACAAATAATCCAATCAAAACTTGGCCAAAGGATTCGAATAGATATTTCTTAAAAGGAGTCACACAAATGGCCAGCAGTAATATGAAAAA
AGGCTCAACATCACTAAGAACCAGGGAAATGCAAATCAACACCACACTGAGCTATCCACTCATCCCACTTAGAATAGTTATTATTTAAA
AAAATAAAATAAAAAGACAAGAAAATGGCCAGTCATGGTGGCTCATGCCTGTAATCCCAGCATTTTGGAGGCCAAGGAGGGCAGATCAC
TTAATGCCAGGAGTTCAACAACAGCCTGGCCAACGTGACGAAACCCCATCTCTACTAAAAATACAAAAAATTAGCTGGGCATGGTGGCA
CATGCCTGTGGTCCCAGCTACTCAGGAGGCTGAGGCACCAGAATTGCTTGAACTGGGGAGGCAGAGGTTGCAGTGAGCTGACTTCGCAC
CACTGCACTCCAGCCTGGGTGCCACAGCAAGGATCTGCCAAAAAAAAAAAAAAAAAAAATATAAAAAAGACAAGAAATAACATATGCTG
GTAAGGATGTGGAGAAAAGTGAACTGTTATACAATGCTATACACTTGGTGTACTACTATATTATCCACCAATCCCACTGCTGGATCCCC
CTCCCCCCCAAAAATGAAATCAACATATCAAAGAGTTCAGATCCCCATGTTTATTGCAACACTATTTGCAATAGCCAAAATATGGAATC
AACTTAAGTGCTCATCAACACATGAAACAGGAATGAAAATATGGTATATATACACAATGGAATGCTATTCAATCTTTAAAAAAGAATGA
AACCCTCAAAAAGTGGGTGAAGGACATGAACAGACACTTCTCAAAAGAAGACATTTATGCAGCCAAAAAACACATCAAAAAATGCTCAC
CATCACTCGCCATCACAGAAATGCAAATCAAACCACAATGAGATACCATCTCACACCAGTTAGAAAAGGCAATCATTAAAAAGTCAGGA
AACAACAGGTGCTGGAGAGGATGTGGAGAAATAGCAACACTTTTACACTGTTCGACCCACTGTAAACTAGTTCAACCATTGTGGAAGTC
AGTGTGGCGATTCCTCAGGGATCTAGAACTAGAAATACCATTTGACACAGCCATCCCATTACTGGGTATATACCCAAAGGACTATAAAT
CATGCTGCTATAAAGACACATGCACACGTATGTTTATTGCGGCACTATTCACAATAGCAAAGACTTGGAACCAACCCAAATGTCCAACA
ATGATAGACCGGATTAAGAAAATGTGACACATATACACCATGGAGTACTATGCAGCCATAAAAAATGATGAGTTCATCTCTTTGCAGGG
ACATGGATGAAATTGCAAGTCATCATTCTCAGTAAACTATCGCAAGAACAAAAAACCAAACACCACATGTTCTCACTCATAGGTGGGAA
TTGAACAATGAGAACACATGGACACAGGAAGGAGAACATCACACTCTGGGGACTGTTGTCGGGTGGGGGGAGGGGGGAGCGATAGCTTT
AGGAGATATACCTAATGCTAAATGACAAGTTAATGGGTGCAGCACACCAGCATGGCACATGTATACATATGTAACTAACCTGCACATTG
TGCACATGTACCCTAAAACTAAAAGTATAATAATAATAATAAAAAAGTAAAAATGATTAAGGACTATAATAGGATAAGAACTCTAGGAA
GATGATAAAGTATGCCCAAAAGAAAGCAATGCTATAATAAAAACAAAATAAAACAAAACAACAACAACAAAAAGAATCAAACCCTGTCA
TTTGCAACAATGTGGACAAACCTAGAGGACTTTATGTTAAGTGAAATAAACCAAGCACAGAACGAGAACTACCACATGATCTCAGGCAT
ATGTGGCATCTAAAAAAGTTGACCTCAAAGACAGTTTAGTACTGGCTATCGGAAACTGAGGAGAGTACTTCCGAGGGGGAGGGGAACAG
GTTGCTCAACAGCTACAAAGTTACACTTATATAGGACAAATAATTTCTGATAATCGACTGCACAGTAGGTTGAGTGTACTCAACAATAA
TGTATTCTGTACTTTAAAATAGTTACACGAGAGGATTTTGGACGTTCTCACCACCAAGAAATGACAAATGTTTCAGGTGATAGACATAT
GCTAATTATGCTGATTTGATCATTACACAATATATATGTATCAAAACATCACACTGTATCCCATTAATATGTACAATTATACATCAATT
TAAAACAAAATAAAATGTATTTTATAAAAATTTTAAAACTTCAACCAGCAAGTTTGAATCTCCTATTCCCAGTTTTGCTCAGCCCTACA
GTGTATACCAACCAGCCTGGCCTGGGGCTGCTGTTAGAGGAAGAAAGAAACATCTGGACTTGGAATCAAACAGATCTGGAAGGAATCCT
AAATTTTCCAATAACATCTCTGTATTCCTTTCATCCTGGTCCCTACCCAGGAAGCAGTCTAGACGACATGATCCTTTTCTAAGAACAGC
AACATTCAGTAAACAAACTTCCTTACTAATGTGTGTGGATCAAGAGATGGTGAGGAAAGTACAGTCACTGATATTTTTGAATCAATTAT
CACAACGCCACCATGCTATTGCTCACTAATATTAGATGCCCATGGACAGTAAGATTCATCGATGGTCCGTCTTTATTATTAGGTCATTA
CTGAGTGGCTTTTGCAATAAAATACACCATTCGCACAATAAAAATATTCTCTAATGCAAAAACATAGCATCCATTGTTTTTAAGGGGCA
CAATAGTGTGCTTCATCAGACTGGAATAGCAACATTGTAATCTGAAAAAAGCTCAACAGTGGTGAGGCATATTCAAAAGCCCCAAGACG
AGAGTCAACAGTCACATCCATCAGAGTTGAGAGGTTGCCACAACTTATGTAATGACCTCTCCACCCCATGAAACACATGTGTCAGCTTT
TCTCAGGAGTCACAGATCTGGCATGCAGCAGGAGATTCTGCATTAGATATATAAAGTCAGCCCCAATCATCCCCTTCATTACTTACTCT
CATCAGACAATGAGCCCTTGCCATGCCTCTGTATATGAGGAATCCACGCAGTTCAGTAAGTAGTCAAGATTTGTTTCCACATGCGGTCC
CAAAGAAGTCTTTAATCTCACAGACTAGTTTTCCAAGTCGCATACCAAAAGGCTGGACCATTCGTAGTGTCCCAAGAATTGGGTTCTCA
CCATTGAGCACACAAACCAAGTCCTCTTTTTATTTCTGCATAGCTGGTTCAGAGGCTGAACACACACAGGAGCCAGTCAACACCATCAG
TTTTCAGCTTAGCCAATCCTTCACTGAGGCAACCCCAGATATTTCGGCAAACCTCTGTAAACACGCTCCAATTGAACTAGTACTGTTGT
TTCAAGCAGGAGAATAAAAGGTTAACTTTCCCCCAGACAGACAGTTGCCACTCTTACATATAAACCACACATGCAGAAGAAATTCGAGT
TGATGCCATCGCCCACCCACCAACCCACCCCCGCATTTCCGCCCTGAGACTAGGATCCCAGCGCCAGGGACCCAGACCCTCCGATTGAT
CCCACACAGCCGTGATCCGGAGAGTGCCCCGCGCATCAGACCGAATCTCGGGGCCACTCGACAGCTTGGGCCAGGAACCATCCATCCAT
TGTCCATCGTGACTCCATGTGGACCAGCTATTTGCCCTGGGAACAGCTGTACACAGCCGGCACCGACTCCCTCCCCACTGCCGCGCCCT
GGGGGAACCCGCCCAAGGTGCCCTCCTCCCCAGACTGCCCTCCCAGCCTCCAAGACGGCAATCCAGCGCCCACTGCTCCCTGGACTCTG
CGCAAGGCCAAGACTCAGGCCTGCTCCAGATTTGAGAACCCAAACAACTCAGAGGGTGAGGAAATTGACCTTGTCACACCAAAGAAAAG
GCAGATTGGGTTTACAGAACTCCATCACCATCATGGTTCGAGTAGACCGTCTGAACCCCTGCATGAAACACTTCCATACCTCCATCCAC
CACCAACAACACAACTGCGCTGCCCATATTTCTCCACAAAGCTGCTCCCAAGAAGAGGCTCCAGAGAGAGGTCTCCAAGAAGAGGTTCT
GGAGGGAGATGCTCCAGCGGAAACGGAACATGAGCAGGATGAGGGGATTGTCACCCCCTCACCTATAGAAAGCAAGCCTGCCCAGTGCT
GCCAGTTCTGACACTGAGGATGTGACCAAGAGGAAGGGCCACACCTTCCTGCACCACAAGAGGTGGAATGACCTGCGCGTTCTCAGTTC
TTGGCCCTGAGGGACCAGGTACCCACCTTCGCCAGCTCCTCTAAGGTCCCCACGGTAGTGATCCTAAGCAAGGCCTTCCGATACTTCCA
AGACCTGGCGTGAACCGAGAAAAACATGTCTATGGAGAAAACGCAGCTCCAACGCCAGCAACAGCAATTGCAGAAATGAATTGCGTACC
TCACTGGCTACTAACTGACCAAAAAGCCTGACTGTTCTATCTTACAAAGACACAAGTTTATTTTTTGACCTCCCCTTCCCCTTTAGTAA
TTTTCACATTTTGGTTATGACGGGACAGTCTTTGCAGTAGGTCCCAGAATGCATTGCAGCCAGTACACACACAATAAGGGCTTGCATTC
TTGGAAACCTTGAAACCCAGCTCTTTCTCTCCCCTGACTCATGCTGTCTCTTCTCTGGCGCCTTTGGCTTCTCAGCAGATAGCTGACTG
AGGAGATTTGGGGTCTGTTTACCTCACTACCTCCGAAGAAAAGGCTGACAGATACTATGCAACAGGTGGTGTATCTTCTCGGGGACTCC
AGCCTGCATGAAATCTCACACTCCGCGTGAGCCTTAGACTAGGAAAGAATCCTCCCTCGTATCTCTGGGGTGATGCTAGAATGCTCCCT
CCTATGTCTGGGGTCATGCAAGGACAGCTCGGCCTGGACACCACTCTCGCTGTGGCTTTTTTTTCCAGGACACACACAACCTCTCTTGG
GTGATGACAAGCTTGAACATTTGATCAACATGACCATTGCTTCACTGTCAGACACTTTACAGTATCTGAGGAGTTGGAAACCTTTAACG
TATATATTGTGATATTAGCTGACACCTCTCCTTCCAGTTTCAATGCTGAGACCCTGAGAACATTTAAAGAGCTTGCACTCTAGGTTCCT
TGTCTCAGAGCTCTCTGGGCCTTCTCTGATGAAGGGACCTTTCTGTCCTCATGAGAGACTTTTGTTTCATTTTGCCTTTGTTGTGCAAT
GGGCTTTACAGCATCCTTTCCCACAGGTTAGAAATGTTTCCCCAAGTTACACGCAAGTGGGGATCCTAGCCTGGGGCCTGAGGAAATCT
TGGAGTCCTGGCTCCTGAACTTGTTCCCTGTCCGACTGGCACTTCAGGCCACCCATCTTATAATCTCTTCTCAAGGCAGATGTAAGTCA
CCTCAGAAGCGAGAACTGTACCGCTTCCTCTTTTCCATGAAGCTCTCATCTCAATCTTTCACTGATGAGTGTGAAATTCTACCGGAACC
ATGCAAACACGTCCACCTTGGGCATCTCCAAGGAACTTGTGGGCTCTGCAGCATACTTGGCTCCCTACCAGCCTGCCATGAGTCATATT
TCTTTTCCAGAAGCTGCACTTGCTTCCTGGTATGTTTTAAAGGAGCCTGCAGGAGCTCTGCTTACCCAATCATGATGGATTTTTGCCCC
AGCTGGACTCTGCATGTCCAAGGAGAATCCAGGTACAGCCTCCATTCCGGCAAAGACATCCAGCCCAGCAGTTGTCATGTGGGTAACCT
CAGGAACCCCTAACCCTGTCCTGGAAAAAGCACAAGCCCCTCCAGAACTCTGCCCAAAATAGCAGGTGCTTGATGGTTCTGAATTTGGA
AAGGGATGGGGGGTGATAAGTACTATCTGTGGCTCTGGAAAACCAGCTGCTACATTCAAATCTATTTTCCATAATGGTTTCTTTCTGAG
GTTCCTTCGTGGCCTCAGAGAATCCCAGAGGATGTTTTGAAATAGCCTCTCTACCCTTCGGGAGCATGGTAGTTTACAGGAACCAACTG
ACTTCTGGAACTGTCTATGGAGGGAGAACACGCCAGGTGTAGGTTACTAATGTCCTACATGAATAGCTTGGTTTTATAAGCTGCTGTTG
GGTATTATGTTGGGGCAGTCTTTTTAATATATTGTATTTTTGTACGCATTTTGCAAAGTGGAGTTAACTGTTTTGTAAAAGAAAAAAAA
GAAACCCTTGGGTGCTTCTTCTGTTTCAAGGGTCTGATTTATTTGCAAAGGCAAGTTTATCTGAAATTTTGTATTATTGATTGCCCAAT
TTTAAAATGTTGCCTTCTGGGACATCTTGACAAAAAATATTTCTCAAACATGAAGAAAATAAATATAATAAAAAAGAAATGTCTGTGGG
ATCAGATGACTCTTCTTGATTTTACCGTTTCCATTTCATAAGCCAACCTTATTGACCCCTTCCTACCTGCTAGTCATTGAGTCTGAGTT
GACCAACTCAAAGATGGAAATATCGAGCCAGATGCTAACAAATTCTCCATQAATCGATTGTTCAGTTTTGACAGAATCCCAATTGCAAG
CCATTAGCTGCTTTTCAAAAGGGATGTACCCTGTAGCCATATTAGGGAGGCAACAAGTCCAGTTCCCCAAGGGATCTCTCTGGGTGTTG
ACTGCTTCCCCTTGCCAGGTGCCCCCATCGGCAAAATCATCAGTTACACACATTTGTCCTGTATGTAATATTCAAGGAGTTGATTGAGG
TTCTGAGCCTCTTTTTCGGTGGCAGCTTGTATGAACAGACGTCAATTTTTTCCTGTTAGAGGGATCTAATGTTCTGAATTTGCCTGCTT
AGTCCCAAGAGATTACTTGGCAAGCAGGCCCCTGTATTTGGGACCATAAATCAGCCACCCTTGCTGACACATATGTAATAACACTGCAT
TGACGACCCTTGAGACTGAGACTTCTAGCCGGCCAGCTGACACAATTATTATGTATATAGTGAAAATATTGAACATTAGAAGGTAGAGA
CACTAATGCTAAATCCTGAGCCAACCACTGCTGACAAATCTCATTGGAGTTTGTTCCACTAACACTTCCCTCTACCTCTGTCTCTCTGT
GATGGGGATGACTTTATATGAGACCTGTAGAATGAGAAATTACAAGCATATCACCCATCAAATCCTGCCATTTATAAGTAGAAGCAACA
AGAACAGTTTATTCAACTTTCCCAAAGGCGTCCCTGTACAAACTCATATTGGCTTCCAAACCCCACTGTGAACCATGCCCAAACCCCAA
CAATTGCTTTTCTGGACTTTTTTCCCTTAAATGAACTCAGACCTAGAGTTCACTACAGGAATTTCAGGGGTTACAGCAAGAGCTGTTAG
GCGATTTCCCTATGTCCAGGCCAGAATGTAGGGATTGCAGGGGAGTCTTGGGATGACGCGAGGGGTGGGGACTGTAAGCCTTTTTTGTA
ATCCCTCTGGCTCTTTGCCCCACCTTGTATGCTAAATCCTCTCCTGTGTACCTCCATTTGTGCAGAACTGTAAGCGCTCAGGGATTCTT
CCATTCTGTCCCTTGATTTCCCCAGCAGCTCTGCTCCACAGTCTTGGTGCTTTCATCTACCTCATACCTATTACCCCTTCCCTCCACTC
CCCAGAAAATCAGATAACATAGTGTCATGGGAGATTATATGTAACAGTTCTGTGAACATGTGAGTCCGTCCCTTTCCTGAAGTACCCTA
CCATGCATATGCCTTTTAGTCTGTCTTGAGGACAGAGAGACTTCTGGCTCACCACTAATCATCTTTCTTCTTATAGCACCTGAAGTGAG
AAAAGGAAGAGAACCAGGAGTTAGGAAATGCAGAGAGAGAGAGAGAAAAGACTCTGTCTATACTAGTAAGCAAGGCATGTTTGCATTTG
GTTTCCAGTAGCCAGCAGTGTGGTTCAACCAAACAAACTTCAGATGGTTTTTTTTCCACTGAAAACTCTATATCTTCACTGCTGACGCC
ACTTATTCAGGTTTGGCAAGTCATTGACTCAGCACCATCACATCATCCCTTCTTCCTCCCATGCAGAGGAGAGTGAACAACAACCAAAC
CATGATTCAAACAGCTTGGTTTAATTCTGCATCCTCCCCCTCAGCTTCATTAATAGGTAGGAGAGTGAGTGGGGAAGGGCCTTTAATGC
ACTTAGTGCCAATCCATTCGGCAAAAAGCATTTGCTCTCCAATCCCGAGGAGTCTTCTCATATCCCCTAACCAGGCCAGTGAAGCACCC
TTGTTTTATCACTTTCCAGAATGCTATGTTTCATCTTCATTACCAAAATTGGCAACAAATGTGAAGAGTTTGAGACTGTACCCTACTTG
CAAGCTCACAAACTAGCATACCTCAGTTTCACAGATGTTGGCAGAATATATGAGATTCTTGGGTCAGAGTTTGTTTTGCTTCATGTTTG
CATTAATTCCCCTTCCCCTCTCCCCCAAATCACACAGGAGAAATGAGGAGCAGCCAGTCAAATGTTGACATACAAGTATTCTTCAAACT
ATAGTTAGAGACAAAAGCTGTTACAATAAATGTAGAAAGGCCATGCACAATTATCTCAACAATATCTGACCATTGTTTCTCCACTATCT
TGGCTTCTGACAAAGTTTGCCATTAAGTATGTCTGTTGATTTTCTGGATATCAGGGCTGAGCAAGGAAAAAGTCATGACCATTAGATCG
CAGTAGCACACAACATGCTTTATTTGGTTGGCCCTTTGAAAGGTTTCAGGAGAGTGGAACTTCCCTCACAGCAGGAGACCTTCCAGAGG
CAGCAGCAAGACCCCACCACCCAGAAQAGGAAGAGTTCAAGGCAACTTCCAAAGGAGAGAAGAATTGGACAGGAGGCTCATGTGTCTAT
GGGATGTTACTCAGCAGCCAGGTGTGGAGTCTCTCTCTCAGAGAGTTCCCATGGGCATCAGAGGCTTCGGGCTTTTGTCCAGCCCCAGA
GTATGTCCTATCTACGGCTACCACATGGTGTGTCTGGTTTTACAGAGCCTCCAATGTCGGTGGGTTCTAAAGGGCCAAAAATGTGCTTA
CTGGGGGCCATATTTAAAGCAATTGGGTGTGTAAACATTTCATTTTAGTTCCAATGAGCTTTGAGCTAATCGTCTTACCTGTCTCTGAA
TATAGGGAACAAGATACAAGGCACCATCTAAACATATTAATCCACTTATTTAACAGTCCATCCCTTGGCCCCATGTTGTTTTACAATCT
AAGTTGTATCCTATTTCATGGAGTCTCAGTTGGGAGCCAGCCATGAAGCATCCTCAAGACCCAGAATGCTATAACGCTACAAATTAGCA
TTTTAAAAGCCAGTTCTCACCCAATCTGCCCACTTGGGCAGCCAGTAGAATAAATTTGAAAGGAGACAATTTTGTTTTGACCAGTCCGT
TTACTCAATAGACTGGCATTTATATCCCCTTTGCTACATGACCCCACCTATCTGTAAAACTTGGTGTTATCTACGCACATGCAGGAAGA
TAATTTTAGCAAGCACACAGCCCCTCTCCCCTCTCACCCCTCCAGCACCCTGACCTAGGGCTATGCAGTTATCTCACACCATCCCCACT
AAAGTACAGTCAGATTCACCAAAGAGTTTCAGTCCTTGGGCTGTTTATCTAATTTCTCTGCGTAATTCCTGCAACATGCCTATCTGTTT
CTCTTAATTGTGGTGTGCTGTTCCACACATATCCTGTTCCTACTGCCTCCAAAATTAGTGATCCTAGGGAAGTTAGTACCATGAGGAAC
TATTTGGAAAGCTCATTTGTGACATTGGGGAGTCACATCTAGTGTGGTTTGGTGCCCGAAAGTACTTGACGCACCCGCTTCCACTGTTG
AATAAAATTCTACTCGAATTTTGCACCAATCCCAAGTGCAGCAAATATTCTTCACGGTTGGATGAGGAGAACATTGTGATAGGGAAGCC
ATCTGATTCCTTTACAGGACCTCCACCCTCCCTCTGCTCCCTCTATTCTCTGTCTTCCACTGAAGGGAAGTTCACATGGCTTTGTGAAG
TTCCACAAGTTCCAGGTTCTGTTAATAGGAGCAGAAAATGGTATAGTCAAACAAGCTTGTCTTATCCACCTTGCATTCAGCACCTGGGT
TAACTAAATTTGTCGTGGGCTCAGCCCACCATTGTCTATACATTTATGTGACATAAGCCTCTGGATAAGGTTGTTGAATGTCAATGGTT
ACCGGAATCGGCTCATCTAAATAAGTGAGCTATATGATGCAGCGTTGCCAGGAAAGAAGTATTACACTATGTTTTCTAAAATCACCTTC
GCACAACCAAACCCTCCATTAGGTTAGATTTACCATCTTATATGTTGCTGTGGCTAAGCCTATAGGCACGTTTCCTGTAGTCGGCAATT
GCATGTTCCTCCTTGCTGTGACTGTTGTGTGTTGTGCTTCAGTTTCCTGTTGATTGCCAATAATAAATAGCATTGATGTGTTCCCATGA
GTGTGGGATAGGTTTTGCAGGTCTTATTGCCAGTCCAAGAAGAACGTTGTTAGTCTACCCACACACCAAATTAGATTCTGAATTATGAG
GCACTCCCATATGAGTGAACAAAACAGGTAAACCAAGTATTTTTTAAAAAAATAGATATCAGCTGGCCACAGTGGCTCATGCCTGAAAT
CCCACCACTTTGGGAGGCCGAACTGGGCAGATCACCACGTCACGAGTTCCAGACCAGCCTGGCCAATATGGTGAAACCCCGTCTCTACT
AAAAATACGAAAAAAAAAAAAAGATTACCCGGGTGTGGTGCCATCCTCCTGTAGTCCCAGCTACTTGGGGGCTGAGGCAGGAGAATCAC
TTGAACCCAGGAGGCAGAGGTTTCAGTGAGCCGAGATCACCTTACTGCACTCCAGCCTGGGCGACAGATCCAGACTCTGTCTCAAAAAA
AAAAAAAAAAAAAGATATCAATTGTATCAAGGCATAGGTCCCTTTTATCTTCCCTTGTATTGCAACATTATTGTGTTTCAGTGGGGGAC
CAAAACCTTTATGGACTCCATTTTAGCGGAAACTTTCATAGGGAACAGCTTTGTATACCTTTAATAACCTCAGGAGTGGCCCCTTGGGA
GGTGGCAGTGTCACTACACCAATAAATTTCCTCCCAAGTACTCATGGACATAAACTGTCTACTGAGACACTGGACACTACTAGAGGGGG
ACGGGGGACGCCAGCAAGGGTTGAAAAACAAACTGTTCCGTACTATGCTCAATACCTGGGTGATGGGATCATTTGTACCCCAAACCTCA
GCATCATGCAATATACCCAGGTAACAAACCTGCACATGTGCCCAATGAATCTAAAATAAAACTTGAAAAAGAAATGACAAGGCATAAAA
ATGTTTTAATGAAGATGAAAACATTCCTCCCAAGTTAGTTTCATTACATGTCTTCTCATTATCCTTTATGTCTTCTGTAGGTTTGGAGT
GTGTATGGATTTTTAAGGAGCCCTCAGAAAACTAACATTGCGCAACAACTGCCCTCAAATAGTTTCTAAGTTTTTAATAACTGTGGTTT
TGGACAAATTTCACAATAGCTATAAGCAAATACTTATCAATGTGGGTGGAGAAACTCCCATGCAAACAGTACTTAGATTTCTCCCTGAG
ATCAAATATCTTTCTTGTAAGAACTGTCTGTGCTGTGACACCACGAGCTATGACTTGTTCACTGTGGGTAGCCAGCATTGCAATTGCTC
WAGAGTTCACTTCCATTGAGTGTTATGTAAAACTTGCAGTTAGTATATTAAGATTAGGTTACGGAAGAATTCCCAAGCTGGTTGTTTTG
TGTGTGTGTGTGTGTCTGTGATTTTTTTTTTAAGAAATGTCTTGGCCCAGGTAATGTCATGTAGTATCCATTGCCTTAGAAGCCAATAA
TTGTAAAGGTATACACTGTGGGTGTCGATTGCACTTATTATAATTCCAGGGATAAGAAAAAGCTATCATTTTATCTATGCAATCCCGTA
ATGGCTTGTGGGTATTGTATCTGGATTATAGTATTAATAACAACACCAATCACAAGCATCCTGGTTGTAATATTTGGTGCATACTATCT
CTGGCCATTCCTTAAACGAGGCTCAATAGATAGTGGCCCATTGCTCCATTTGTTTAATGGCACATGAGTTAATAATAGTGGGTATCATC
TGATGTGAAGGAAAAGGCGGCCCACAGAGCTTAGAAGGCATCTATGAGTCACAAGTGAAATTGTACAATCTGACTTAGCTGGAAGGATG
TTACTAAGTGGTAGTACAGAGAGGAGGAGAAGTGCTAACCTCAGTGCTGATGTCTGTGGACTGACCCAGTGAAATGGGAGCATGGTTCC
CTTTGTTCCCAGTCTCACCTATGTTGGGTCTCCCTGTGGTGTGTATGAGCCATGCTGTTTCCATACATACTCTGCCAGCAGTAGGGTGT
GGATTACCAGGGATAGGGTGATCCACCAATCGAGGTATAATTQACATTGGTGAAGGTAGGAACTTTCTCAGGTAAGCAAAGGGCAGAAC
AACAGCATAGGCATAAATGTTGGAGGTCATTTGCAGCCAGCAGTAGGCAATCTGATTGTCCTCTCGGAGTCACCCAAATGGTGTTACCA
GATACAAGGGTTGTTGGTTCAATCTCCTGGGTGTCCGCAGCACGTCAAGGTTTGTTCCATAGTGCCCTTAGTGGAGCAAAGTACATCCC
CAGAAAATGAGGATGCTTCCCTCTCCAGATAGTGGTGGGTCGTTTGCTAGTTTTATGCTGGGTAATGAGCAGACTAAGACTGAGAATGC
ACCCAGAGTGAGTGGTCCATCCCAGATCCTTGTCAGGATTGAGAGCGGAAAAAAATGGCCAGGCTCTGGATTTTCTATGAGGGAGAAGG
AAGAGGTGAAAGAGCCAAGTTCCTTTGCTGTGGGCACTCACTTCAGGGCCCCCATGTTCGTGAATACTGGTAAATCATGTTGTGTCCAG
TGGGTACATACATTACCACAGGTTGCAGCATCTCTGTTGGTGGTCTCCTCATTTGCTGTGAATTAAAATATCGTGCATTTATGCCAGAG
AGCTATCGATTTAATGTAAGCATGACTATGGGCAGTATTTTTGCCTACGGGCAATGCTATTGTCCTGCATGTTCTTTCAATATCCCTTG
TTTTAAAAAGCCCATTGTGTCATTCTGTGACACCTGCTACTGTAGGACCAGAGAATAAATCACAAATCCAAGCAGTGTTCTTTCGTAAT
GCCTATGGTTGAATCTTCTTTGCACAGAAGTGAGAGTCTTGATCTGACTGGATAACTTTGTGTAACTTTTGTACTCCCAAAGGCCATAC
AAACTATTTTATCAGTTCCTCAATAACATTAGCACAGCTTCCTGCCTTTCGTGGGTAAGCCAAGAGTAAGCCTGTGTAGGAATTACTAC
ATTGTTGTGGCATATGTAATACTTCCTGCTGCTTCAGAGAGGGGCCCAATAAAATTGACTCGATTTGCCACCTCTCATATAGCCCTTCT
CCTGTGCAGTAGGTCCAAGTCTCTAGATTTCTGGTCTATGGTACGGTCCCTAACAGCTTCATGTAAATGTCTCTCTGCTGGGTCTATTT
CTCATTAACAGTTGATTTTCATGGTCTCATGATGCCCAAATGCCCTGACCTCTCCTTTATAGCGTGAACGGTAGCTCTTACCAAAGTTC
TCCTGTCTCCCTTCTTTTCAAAAGCATTAACTACTTTGGTACTTTAGCTAGTTTGTCAGTTTCCACGTTGTATTGTTTGCTGTGAGCTG
AGGTATGGTGTACAAAGATGTGCACAGTGGAGGCCAATCTGCTGATATTTTCCCCCACATCTCCTTACCCCAAATGTGTCAGCCTTGTA
TGTTCCAGCCCTGATTTTGTCCAACTCCCAACCCAATAGCCTATTAGCCACTGACCAGGAGTTGCCAAAAATGTGAAACTTAGTAAAAT
GTTTGTCGAAGGCCTTCTCTATTTCTAGGGGTGTTGCACACAGCCTCGCCCATTGGGCACTTTGGCCCATCCCCATATGGAAGTTGGAG
TATTTGAATAAGGGTAGTAGGCTGTTGCTCTCCTTTATCTGACTCCCTTATACCCTGTGCTCACTCCATTGGTAAAGAAAAACATTAAT
TTTCTTGTTATGAAAGTGCTGGCCATACTCCCTCCATTCCCCAATGAGCAAGAGCCAGCGGCATTTCCAGGGGTTTTGGCAAGTTAACC
TCACTGACAGAGTATGTGGCCATTTCCTCACACAGTAGTCACACTCCAGGAGGGCCAGGTGTAGTTCAATCCTGTAGATACCATTTTCA
TTTTAATAAAGAAGCTTCTCTTGCTGATCCAATTTTATGGGCAGCTCTGTCCATTACCCAGGGCATAACACCAATCTCGGTCCATAGGG
TAATTTTCTCTGCCCCACCAGCGCTTCAGTTTTGATAAGTGCCCCATATGCTGCCAGTGGGTGTCTGTCTAGTGAATACTTTAGGCTGA
GGCTGAGAGCTACTTACACCAAAAGCCCATGGCTAGGCATTTACCTGTCCATTTGGTCCAGAGGCTCCAGGAAGCAAATTTCGGACCAG
TGGAAACTTATAATTGCAAGTCTTGTCCTTGGGAGCACTAGAGGGAGAGCCAAAGTATTAGCCTCCTTTAGCTCAACCAGTGACTGTAG
ATGTTCAGGTCCCTACTGAAAAAAGGAAGCTTTACAGGTGTCTCGATAGACAATTCAGCAAGGGTCAGCAAATATTGGAGGTGAGCAAG
GCATTGTGCTATAATTCCTAATGAATGTTCGCGGTTCTTTACTGATGTGAGCCGTTTAAGCATGAGGAGCTTGTCTTTTATCAGATTGC
AAATAGTCTCAACACTATTTCTGTCCTGTCAAAGTGTAGAGATCTTATTCCTAGATTTAGACTGCCCTTAGGAATCCGCTCAGATAATG
CGCCAGCGTTTGTGGCTGACTTGGTACAGAAAACAGCAAAGGTATTGAGGATCACATCGAAACTGCATGCTGCCTACCGACCGCAAAGT
TCTGGAAAGGTGACCGAGTGTGGATCAAAGACTCAAACATAGCCCCTTTGCATGCACGGTGGGAAGGACCCCAGCCCATCATCCTGACC
ACTCTCACTGCTGTGAAGGTACAAGGAATCCCGGCCTGGATCCACCTCAGCAGTGTAAAACCTGCACCGCCTGAAACTTAGGAGGCAAG
ACCAACACCCGATAACTCAGGCCGTGCTCCGCCTGTCAGATAGTAAATCCCACGTGATGTACCTGCTGTCAGCAGATCTAGGCTTTCTT
CTTGGACACCTTATACCCACACTCCTCCAGGTGCCAAAGTAGGGCATCCGTTCCCTTGGCAAACCCGACTGCTGTCCGGTGTCCTAGCA
GAAGGTCCTCGACGTACTGGAGCAACACCCACCCTGGGTCTCTGCCAGGAAACTTCTGGAACTCCCCAACCAATGTTTCCTCGAAGAGA
TGGTGGGGGACTTCTTGAACCTTTGGGGAACCCCGGTTCAACTGTACTGAGTGGTGACACCCGACCCCAGATCTTCCCACTGAAAGGCA
AACAGTTTCTCGCTCTCAGCGGCTACTCTGATGCTAAAGAAAGAGTCTTTCACGTCCAAGCAGGTCAACCACCTGTCCTCAGCTGACAG
CAACCCTAACAACATGTACAGTTAGGCACTGTTGGATCTAAACTCACTGTACCTTCATTAACCAAGCACAAGTCCTGTACTGGCCTGTA
GTCCTTGGTCCTTGGCTTGGGAACACGCAGGAGGAAAGTGTAGTATTCTGAGGTCTGAACAGGCAGGAGTGGAAAAACACCCTTCCGAG
TTCCTAGTGTAGCCTTCAAATTTTACAGACTTATACCTTACATCGTCTAATTCCCAATCCACTCTCAGTGAGACGTGGTTTACTGAGCG
TTGTGATCAACTGTCAAACAGAGTCTTGCTCACGTTCTGCCAGGAGGATGTCACCGATATAATGCCAAAGCATACTTTCTGGTGAAATT
GAGGGGCAGGTGTCTATGTCCTGTTGGCAATGACTGTGCAATTGGAGGGCTATTAAGTATCCCCCAAAAAGCTAGCATCACAATGAAGA
TGAGGGGAAACAAATACCCCAAAAGCAAGTGGGTAAAGGCGTACTATTGACTCTCAAAGGTGGAAGTGAAATGTGTTTGACTCAACTGT
GAAATTTTTACTAAACAGAACATGTTACCCAAGTCTACAACTCCAGATTCAAATTCTCGTGGCTTTAGATGCCCTGGATTAATATAAAC
TTACCACTGAGGTTTTGAATGACACCCATTAAGGCAACAATGTCCTATACAGGTGCTCTAATAAAGGAAACAGCCCTGCTGAACTCTCT
ATGGTCTATAGTCTCCACTGATTAGTACTGTCTCTTACGGCACGCCATATGGGGAATTGATACCTGAGGTAGTAGGAGTCAGTGTTCCT
TGTTTATCTATGTCACCTGTGACAGGCCATAATCCTTCAGTGTCCTGCTTCAGCTTCTGCTGTGGTATATGAACCACCTGAACCAGCAG
GGGCACGTTAACTGGTTCCCTCCGGGAAGTGCCCACCAGTAGCATAAAAACCTTAGTCTTATGTCATGAGTTTATCTGCGGCCTCAGGA
GGTCTCTTCCAATAATGGGGAAATGGATAATCTTCGAGGGCAGGCAAGCAGGACAGAAGCATATTTTTAGCAAAACTGAGCCAATTTTA
ATAGTTAAGAGGGGAATTATCCCTTTAATTAATCCCTTTAATTAACCCCCTTCCAAGCAGCTAGCCTACTTGCCAGGAGGCGGTTCCTT
TTAAAGCATGTGTGTGTCTTGGCATTAGGCAGATTTGGCCACCCCTGACTGACACAGTTACCCAGTGTTGTTTGTCAGTGCCCAAGCCA
CAGTTAATATGGTGAAAGGGCAACTGTCTGTCAGAAGGGGCCTTCCGACACTGCAGCATCCCTCTGTGAGAGAAGATTCTTAGCCATTT
GGCAAGCATCAGAGACAGGTGTGGAAGATAAGGTTACCTCCAAAGGGAAAGCCAGCTGGGGTGGGAGGGGGTCGTAATCTAACCCTAGT
AAGTATTGCAATTGCTCCAAACATTCACCAGAGATCATCCAATTTGGCACCTTCTTGTGCCTTAGCGTCTAACATAACCTCTTTTCGTC
ATCTCGTGGTTGATGCATACATCCGATCCTGGCCACACGTGCTTTGTTTGGCCCAGGAGCCCCAGCCTGAGTCCTGATAGTAACTTTGA
TTTAGGCCACGTAGGGTATTATAGTGTTAGCACGCAGCACCTGACTCTGACTTTGTCTTTCATGAGTTTCTATTAAATCCAGTCCAGCT
ATGGTCTCTTGAATTGTTTTAGCAGTAGCCACCATGCCCAGTAAAGAAGATTTTCCCAGGAGCCCCGCCCCCCGAGGAACAATTGAGTG
GACTCAGGACTCAATTCAGACTCCTCCAGTCTTGTCAAGTCATCCTGCCCCACCTCATTTGTTGCCTCCACTCCAACATGTCCAACTCG
TGGAGGAGATGTTTGGCCTTGGCTAACCTTTTCCCTGGACAGTCTTCCCAGGCCATTATGAGGTCTAATGGTGGGCAGTCTTCTAATCC
ACAAAATACACATCAGCAATAAAGATGTGAACAGAGGTCCAATGTGGCTTCTTCCCAAAATTCTGCTCTAATTATACAGGTTCCTTTAC
TGGGGACACAATTTTTCCATCATGTACTATCGCTCCTAATTGTTTAATTTCATTCCCCATTAGGTGGAATCAGTTCCCTGCCAGATGCA
TTCTCTAGAGCCAGGCAGCCCCTTGTTTATTTGGTTCTTGGCCTGGGTACTTAACGACAGACTGGGGCTCGGGTAAGACAGAGGATAGG
ACTCCTCCTCTGAGCAGGAACAGACCCTTCCTTCTTGGCTCTCCCTTTTTTTCTTTTCCATTTTTATTACTTTCTGGGTGGCTACAGGC
AAAGCAGAGGGTAACTTTTCCCAGTCCCTACCGTGGTGACACTGTGCTGCCTTTTCTTCTGGAGAGCACCTATTCTCCTCTCCAGGGAC
CTCTTGTCCAAACTGTCTCTCCCAAGAGTTTGGGCTCTTACCTCTTCTCCAGGTGGCATCCTGGTGATAAATTGCTGCCAGCAAGGTCA
GCACATGCCTCATCGTTAGGAATCCTTCTTCCACCTGACAGCACCTCCTGTAGGGTTCCGGAAAGGGGTAGGAATATAAATTTCTTTGC
CTTCAGTCACTTTAAGTACCACTCTAGCTAGCTCAGTCCAAAGGTTGAGATATTAATATCCCTCTGGAGAGGGGATAGCATGATGACAA
CTGCAACATCATAATTGAGTGTAACAACAGTAGAGCCATGCTACCTGCTTGCAAAGGCCTTTCCACCAGCTCCCCTTAGGAGACCAGCC
TTGGCCTGATGTCTCTGCCTTATTGGCTCATTTACTTGCCTATCTGTCTCCGGGGAGGAGGAAATCCTCACATCTCAGACTACCCCAAA
TCCTCAACCCAGCCACAGGGAGCACTTCCCACCATAGCCTGTTATGCAGTCCAGGGTCGTTCACCCTTATTCCAGAGGCATGCACTCCC
TCTCCTTTTCTATTCAGGTACACAGGTGCACAATGTCCAATGTCAACTGAGGGGGGGACCCACGTAAGTGGGCTGACCTCCTTCCCAAG
TACCTTGGCATCTAAGACCCCAAAAGTCAGTTCTAGTGGTGTGGGTTCACTCAACTACTGGTGCTACCTCTGGGCCAGGTCAATGAGGT
ATAGAAGGAGACTGCAAAAAGTATATTGTTGCGGTACAGCAATGAGCGATGCTAAGCAAAACTGCTACAGAGGCATTCTAAATGTAGAG
TCTGAGCACTTAGGCTGCCTAGGACTGATTTTTCAATTGATTGTCCTACTGACTGGGTAGACGCTATGCCAAATTTCTTGTTCACCAAA
ATCCCATCCTCCTCACCACTTGTCGTGAGTTCCAAGATTTCTGGGCTGATCAATGAAGAAGACATGACCACGAGGTCGCAGTAGCGCCC
AAAAACATTTTTTTTTTTTTTTGAGAAGGGAGAAGGAGTCTCACTCTGTTGTCCAAGCTGGAATACAGTGGCGCAACCTCTGCTCACTG
CAACCTCCGCCTCCCGGGCTCAAGTGATTCTCCAGCCTCAGCCTCCGAAGTAGCTGCGATTACAGCCTCCCGCCACAATGGCGGGCTAA
TTTTTGTATTTTTTAGTAGAGACCAGGTTTCACCCTATTGGCCAGGCTGGTCTCAAACAGCTGACCTCAGGTGATCCACCCGCCTCGGC
CTTCCAAAGTGCTGAGATTACAGGTGTGAGCCACCGCGCCCAGCCAAGAACCTTTTTATTTCGGCGGTGCTTTGACAGGTTGCCAGAAA
GGGGAGGTCTCTCACATCAGACCTTCCAGAGGCAGCAGCAGAGGGCCACTGAGCAGAAGGGGAAGAAGGCAAGGTAACTCCTGGGGTAG
AGGAGAATAGGAGAGGGCCTTATCTGTCTGGGCGATATCACTCAGCAGCAGGAGAGGGAGTCTCTGGGTCAGAGGGTTCTGGAGCCAAG
CAGTGGCATCGCGTCTTTTATAGCCCCTGTCTTTTCTTTAAATGTGGCTAGCAGATGTCAGTGCAGTTTTGTGGGGCATGCAAGCCAGG
CAAGCCCTAAGTGGCTACACATATGCTTACTTGAGCTATATTTAAAGCAATTGCATATGTGAAAAATTCGAGTTTGGCGTTGGCAGGCT
TTCTGCCTGCTGTGAGGAAGTATAATGGCAATATAAAGGCTAATATATGGAGGTCATCTTTAACCCACTGATATAATAACATGCCACTT
AGTCAGCCACTCTGATTAATCCGACTAGCATGGTCTGAGATAAAATCCATTCAATCTGTACTGTCAACAGTACTCTAAGCAGCAAGGTG
AGATCAATCTGCAACGTTAACCCGAACTTAAAACGTTAAGCGCACACGTACAATACTACTGCTCCTGAGTTTTTATCTAAGAGAAATGA
AAGCATAGGTCCACATAAAGACTTGTATGTGAATGTTTATAGCAGCTTCAATTGTGCTAGTGAAAAACTGGAAACAATCCAAATTACCA
TACAATGAAATACTATACTATGACAAATAGTAATGAACTATTGATAGAAATAACAGCATGGGTGAATCTGAAAATAACTACGCTGATTT
TAAGAAGACAAAAAAGAGTATACAGTGTATAATTCTATTTATATAAAATTCTATAAAGCACAAACTAATCTATAGTGAGAGAAAGTAGA
TCAGTGGTTGCCTAGGGACAGGGATGGGATGGGGATGGGTAGGGAGGGAAGCAACATAAAGGGGCATGAGAAAGCCTTAGGAGGTGAGT
AAAATGATCGTCATTTTGAATGTGGTAAGGGCTTATGATTATTGGTAAGGAGTTATGCTTATAAAATATAAATAATTTGTATTTTATAA
ATTATATAAATTACATATAATTTCATTTAGTTTTAAATTTAAGGAATGGCAATATGTCTTGCATCAAAGAACCCTCAAAACAACACCTT
AAGTACTGCAAAGTCTTTGACGATGTATTTGTTGAGTTAACTTATCTTTAAACATCAATAATAAATAAAATGGTCAAGACTTATTTGTT
TCTATGTGTTTATTTTAGTGTGCTATTGCTTTTTCACATTCTGGGTCATATTTTCAAAAGTATTATTTCACATTGTGTAATGTATTTTT
TTTTCACTATTAGTAAAGAGCATCTCAAGTAAGGTCCTACTTTCTACTTTGATGGTATACGAGCATGTTTGACTCAGATATGAACCACT
TCTGGACAGAGTTATGGGTGAGAAATCTGATATTTAAGCAACACTCTGGATTACTTTCCACTGGTGAGAATAAAAAAAAGTGATGAGCA
CTTTTTCCAAATACCACGTGATGAATCAAGTACTCAAACATGCTAACTGCATTTAGACTATTCTATTACCAAAATATTCCAATCAATGC
CTGTGGACAGTTTGTAAACTGTCATTCCCAATGCCTCCTCATTCTTCAAGTTCCCACTGAAAGGTCTCATAGCTTATCAGACCTTTGGG
GGAAGTGCTTTTTTTTTTTAATTAAAAGTTAAGTCATGTGCAGGGCAGAGTGAGGCAAAATGCAGCGAGTGGTTAGGACCTAAAGACCT
CTCCTTCACCTGGGCCTTGAGAGTTTCAGAGAACCTCAGGAAGAAAAGCCAACCATCTGAGTCCAGTGGCCTTTGAAGCAGTGGTCTTG
CATCTGTACATGTGGGATAAATGATGTGAAAACCATTAACTCTGCTGCTGATCCAGCTGCAGGGAAAAGCATTTCCTCTGATCAAGGTG
AACATCTGGCTGGTGGACCTCATTTTGCACCAGGTCCTGATCGATGAGCTGAGCCTGAGTCCACAGTTCACCTGATGGAGAACTCAGAC
TCAAAGCCCAGGCCCAAGTGAGGCTGTCTGACTGCTGCTTGAGGGCCTAGGGACCACACCCCTGGAGTCAGGACGGCAGAGGTGAGGAT
GACAGTGCAATGGCCAGAAGGTAAAGATACATGTGAATATTCTATCTCCATTAGGTCACATGGATGAGGCAAGAGAAAAAAGTAGCATA
TACATTTTTGAGAAGTTAAAGCTGATAGGACACAAAGATGGTTGATTGTTCTACAGACGTGGAAGGCAGGGGCTTAGGTCCTGTAAAGT
CCCCCACTGAGCAAATAACTGAGACAGTACTTCCTAGTGATGCATTCTGCTGTCAAACAGTGTGAAATTTTCCTGGAGAAGGCTCAGGC
CATCACTCCCTGGTCCATCTTCTCCCCCCACAGAGATGCTCATCCAACACCCTCCACAGGGACAGCATTGCACAAGTGATTCCCTTTTT
AGAAATTCTTCTAAGCCAGTCAGAAGAAGACAAGCTTGCAGATGGAAGGGAATCAGAACCTGGGCAATATCTTGTAGTGGCCGACCTAT
CCCTGTGTGTGGTTTGGAGTGTTTTCACTAAGACAAAACAGTGGGAGAAAGAAAAACCCTACACCCATACAAACTTTGAGGGAATACTG
GAGCTTATTGTAACAGACACTGGTCATGCTATGAAATTTTATTACCTTTTCTGGAGCCGTCGCACGGCTTTGTCTTTGGGCTGCTTTCC
GTGGTGCAGTAATTGTCAAGCTTGCCAAAAGTGTGACAGAATCACGGAATTACCCTTGGCAGAAACGCAGAATTACTCCTAACCTCTAC
TCTCATCCTCCACCTGCACCAGACAGACAAACAACTTCTTGAGTTTTCAGAAACTACCAAATCTTGGCTCTAGCTCCGCATCCTTGGCC
AGAGCATGGAAGGTTGGGGTGAGGGACACGGTGAACAAAAACTGCATCCTCGAGCTGCTCGCACTTCCTCTTTCCCTCTTTGGTCTTGT
GTGTGCGCTCACAGGTTACCAGGCAGGGGTCACTAAGGTTGTCAGTGGGACGATGTCAGCCCTACTCCCTTCCTCCCAGGCAGCTCAAC
ATCCGAGGCTCCCAATTCCCCTTTCTCCTCCAACGAAAGATACCACAGCTGTGTCATTCTCCTTCAGAGACTGCAGATTGGAGGTCTGT
TTTTCCACAAAACCTACTTCTAAGACATTTCTAGCAACTGAGAGTGACTGATTGGAAATCTAGTGGTGACACCTACCCATGGAACAGGC
TTAAATAGCGACTCCTCCTTGTAGTGAACGAATTTCAGATTTCACACAAGAAGGGCAAAACCATGGGCTATTTTCTCCCATGAATTTTT
GCCTATTTTGGGCCCTAAATCAAATTGACAAAAGTCTGACTAAAACACTCAAACTGCCAATATAGTTAGAGATATACACTTTGTGAGAC
TAACTCGGGAATCTACTGCCCTGCTTCTGCAAGGATAAGGCTCTGTAAGGCATGACTTGAAAAATGATCCAACCACCTTTTATCTGTTT
AACTCTCGTGCAGGAGAACAGAGCAGCATTTAAGGTATAAATTAAATTAGTCTATTCCTGAAGAGAACATTTGCTACTCACATTTATGG
TGGAAGATGCAATCTCACAACACAACAAAGCTCTCCTTCTGAGTGGAGCACGCCAAATACTGGGTAGGGCTAGACTATAAGCTCACAGG
CTCTATGCCCTAAGCTACTGATGCCCCTTTGTGCCAGAGGCTGGGTTTTGCAACAAACTCAGTTCAGCTCTCGGAACACTGGCCCCACC
GAGAATCCTAAATAGTCAACTAAAATCCCTGAAATACCCAGGCCTGTTTCTTTAATCATTAGTATTGTTTGCATACCTTTAGGAGGTAC
AAGCGCAGTTTTGTTACATGGTTATATTACCCACTGGTGAAGTGTAGGCTTACCAAAGCAAAAACAGGGTGGTAGATAATCTCCCTTGT
GCAATCAACAACTATAACCCTGAACACGTAGGGTGCCTACTATTCATACTGACTACAGAGAGCCTGGACTCCTCTAACCTGATAAGGAT
ATGAAGCCAGTTATCTATTTATTACCTCTTAGCTCCAAATTCACCAGTCAATATATATTCTTCGATAATA0AG0GATTCTTTAAGCATC
TCTCCTTTAAAGTAGCATCAAAGAGCACGATGTTACACTTACTTAATAGAGGGCTCTGGAGGGACAGACATTGAAAAAGGAAGGGTACT
TCTATGATTTCTGGCTCTCAGAGGTGTGGAGGTAAGGACATTCAGGATGCTTTGTCCCAGGCCTGGGCCCAGAACAACTGTCCCTCAGT
AAACCTGCAGTCCTGGTGTGCCCTGATGATCAGCTTCTTGTGGCCCTCTTGATAGGGACATCCTGTACTCCAAGTCATCTGCCCACTCC
TTCCCTCTGCTCACTTGCTCCCCAGCCACACACACACACACACACACACACACACACACACACACACGACCAGGTTCATGTAGTCCTTT
CAATACTGCACCCCTCTCAACACCCAGCCCCCACCCTGATCCTGTGCAAGGGGCATTGTTTACTCCAGGCCTCCAACTGAGACAGGACT
CCCAACCCCTACTTCACACCACATACTAGGCACCAGCTATGGCTTGTCCACACTACCATGCTCCAAAGGGTGCCTTCTTGCTTCCTCTG
CGACCGTAGACCAGTTCTGCCTCTGTAGACCAGCTTAGGTACTGGCAAACCAGTGGAACTTCTCTGCCATCCAGTAGGTTCTTCTCCAA
TTAGGTATAAATCCATCCTTGGGGAGAACCTTTCATTCTAAGCTTGTCTTTCCTTGGGTACTCTCTCCCAGCCCCAGAAAAACCCCACA
TAGAGTTGTCTTGTATCCTATACAAGATCTTTTATCATCGCTTAATGATTCTTTATATTAAATTTTCCCCTTCTAGATCACTGTGTGCT
TTCTATCTCCTGATTGGATCCATATTGCTGCAGAACACAACATCTACAGCAAGCATCATTCTTAATGGAGAATCACTGAAAGCTTTCTC
TTCAAGAGCTACATCAAGTAAGGATATCCACTATCACCACTTCTATGCAATAGTTTACTGGGGCTAGGGGCGGGGTCCCAGGCAGAGCA
GTAGGGCAA0AAAAA0CAATAAGATATTTGGAAACGAAGAAGAAAGACTGGAACTATTTTCAGATGATATGACAGTGTATATAAAATTC
TACAAAGTATTACAGACAAATTATTAGAATCGATAGAAGAGTTTATATGCTTATAGGATAAAATCTTAACGAACAAAAGCCAATTGTAT
GCTATTTAGAGCTATAAATTTAGACAATAAAAATTTCAAACATATGATTTGCAAAATATTAAATGCCTAGGATAAAAAAGTTGCCTTGT
GGGAAAAATATAAAATAATATTTGGGCAAATTAAAGGTGATCTTATTAACAGAAGAGATAGACCATAACCACCAATCGAACATTCAATA
TTGAGAAGTCAAATCTAAAATATCAGCACAATTCAAATTACAACCCCAAACTAGCTGTTGGTTGAAGTTTTTACGATTAGTTTATAATT
TAAATGGAATTGAAATGGACCAAAAGTAGCCAAGACACTTTTGAAGAGCAAGGTAGAATTGATTTGCCTAAATCCAATATCAGAATTGA
CTATGGATCTGTAGTAATTAAGACAGTGTCCTATTTCAAACAGACAAGTCCTTAATAGAGAGTATAGAACTAGCCCACATATGTGTAGA
TACTTGATTTATGGCACTGTAAATCAACTGGCAAAAGGCAGACTTTTCAATAAGTGGTTCACTGAGAAAACTGGATATTCACTTACTTA
CATACATATATACATAAATGGAACTCTACTTAACACAATACACAAACATCAATTGCAGGTGGATTAAGACCTGACTATGAAGGACAATA
GCAGACATATTTTAGAAGAAAATATAGAGAATGCTTTCATGGTCCAGGGTTCCTTAAAACAGAAAAGATTTCTTAAAACAAAAAAGGAG
TAACCATCAGGAAAAGAATGATGAAATTGACTATATTATAATTAATAGCTTCTGTTCACAAACCGGAGCACAAAGCAAGTGAATATACA
AACAGAGCAAGGGACTATTTTGGCCGCAGGTGTGGAAGAGTATTTAAAATATTTAACAGACTCATTAAGTCAGTATCAGATGAACAATA
AAATAGAAACGTGGGTTAAAAGAGGTAAATTTGAATAGGCCTTCCTCAAAATAGAAACTCCAAAAGGCTCATTAACACAAAAATGTGCT
AAAGCTTAATAGTAATTTGCATATTGCCCACCAGTAGACCGCCAAAATTTAAATTCGGGCAATACTTGGTGTAGGTGAACATGTGGATC
AGTAAGGACTCTCTATGCTGCTGGTGGGATTGACAACTCTCCCAATCACTACAGAGAACAATTTGGCATCATCTACTAAAGCTGAGGAT
ATCCATTCCCTATGGCCCTGTGATTCTATTCTTGGATATATACCCTAGAGAGATTTTTACACGTGTCCCATTAGAAATGTGCAAGAATG
TTCACAATAATCATTGAAAAGTAGAAGCAATCCTAATCTCCATCTGCTGTAGAATGGAGTGGAATAATCATACAATAATCACTTTGCTA
TAAGCATATGCTGGAAAGACAGTCGATGGAGACAAAGGATCTACTTACTTATAGCTATTATGAACAACATGGACGAGTCTCTCAAGTAT
AATACAGAGTGAAAAAAGCAAGCTATAAAAGGAAGCATGCCGTATGATTCCACTTATACAACTTCATATCAACTCTATTGTTTAGGGAT
GCGTACAAAAGTGATAAAGCTATAAAGGAAAGCAGTGAAATGATTATTACAAAGTCGGAGCAATTACTATATCTATATAGGAGAGTGTG
GTTTCTGACTGGAAGGAGTACAGCAGCTGTATTCTGGGAGGTTGGCAGTGGTCTATTGCTTGACCTTGGTCCTGGTTAAGTAGGTGTCT
GCTTTTGTAAAATAATCTAAACTATACAGATACGTTATATGCACTCTTCTAATTTCAGATTTGAAAAGTGCTTCCTCCAGAAAAAGTAA
TAGAAAGAAAAGTATGTGGAGTAGAAATTTTGATGAGGTTCGAATAGTTATGGTTGAAAAACTTGATTTTCAAGAGACCAAGTGTCCAC
TGTTATTAAAAGAGGGCGAGAGAGCTGGGAGTATTTAAACAACAGGTTACACCCCCCCACCCCCACCACCACCAATCTGTTCATCACTC
TAAGCCTTCTCATCTCAGATATTTCACCCTATATTTGTGTCAATTCCAAACTCATTCGTGACAGACAAATTGGAGGATTGTCGTCATCA
TTAATAAGGACATTAGGCACCAAAGGCCCAGCAAGACTGGGCCACCACACTTAAATGTGAGCAGGTACACCGGGCCGAGTGGACAAGAT
CGTCTGTGCACATTACTTGGCAGAGTCCATTGAGTGACTGAGGGTTTAACCTTAACACATTTAACTGGAACTCCTCCCCAGAGAATGTG
CTGGAGAATGCAGAATAAACTGAGCAATGCCCTCATTGTGCTATGAGAGTGGACATGAAGTTTATCATGGTACAACCGATCGTCGAAGC
GCAAAGAGTTGGTTTAGGTTGTCCACTGTATAGGGAGAAGCCAGGGGCGGGTTGCGTTTTTTTCCAGATTTCAGTATAAATTGTTTCAT
CCTCCAGACTTTGCATTTGTAGGCTGAAGAGTCCTAATCTGTTTTGGCCGCATCTCTCACTCATTCTAATCTCTGTTATCAGCAGACAA
AAATTGAACTATGTGGCAAATTGTACC0AGCACTTTGCTCAAGACAATTCTGAGTATGGAACTTGTCCCGCGCAGAGGTGGGAAGGCCA
AGGGCAATTTCCGCAGAAATGCCAGCACTATGGTGCTTTAATGCTGACACAGAGACAGTTTTTTCTCTCAGGAATTGTCTTTCATCCAT
CAATCCAGCCAGGTAGCCAGCCAATAAACAAATTTTAACACACAAAAAGTATGAAGCAACATGTCTAACACTGGGAGGGGCTGGCGTGT
GTTTATTTGGGTATATACAATGAGTCAC0GATTAAAATTGCTTTTACCAGATAGATACCTAGGTGTTCCACAGCATTATTGAACAAGAT
GTCGTCATTTCCCCATTACTTCAGAAGACCTCCTTAATTACTTATATTAATTGTAGAATATATTTATGCATACCAGGCTCTACCTCTGG
GCTTTCTATTCCGTTCCAGAGACCCAGTACCAGAATATTTAAATCACTGTGGCGTTGAATACATTTTAATATCTGATAGGGCCAGTTTC
TGCGCATTGCACATTTTTTTTTCCCCTTTCACGAACGTGTTCACGCCCGCCGCAGGGGGCGGGATCGCCCCTCCTCCTCGGCTCTGGTT
CCAGCCGAGCCTCTCGGACGCAGAGATGGAAATCCCGAAGCTGCTCCCGGCTCGCGGCACACTACAGGGCGGCCGCGGCGCCGGTATCC
CCGCGGGTGCCGGCCGAGTCCACCGAGGCCCTGACTCGCCGGCTGGCCAGGTCCCCACGCGCCGCCTCCTCCTGCCCCGGGGCCCCCAA
GATGGCGGGCCCGGGCGGCGGCGCCAGGAGGCCAGCACGGCATCACGGGGCCCTGGCCCAAGCCTGTTCGCGCCGAGGCCCCATCAACC
TAGCGGCGGCGGCGACGACTTCTTCCTGGTGCTGCTTGACCCGGTGGGTGGCGACGTCGAGACCGCGGGCTCCGCTCAGGCCGCAGGGC
CTGTGTTGAGGGAG0AGGCCAAGCCGGGCCCGGGGCTCCAGCGGGACGAGAGCGGCGCGAACCCCGCCGGCTGCTCTGCGCAGGGCCCC
CACTGCCTGTCCGCCGTTCCCACTCCGGCCCCGATCTCCGCCCCCG0CCCCCCCGCGGCCTTCGCGGGCACAGTCACTATCCACAACCA
GGACCTCCTGTTGCGCTTTGACAACGGCCTCCTCACCCTCGCCACGCCCCCACCACACCCCTGGCAGCCAGGGCCCGCTCCTGCCCAGC
AGCCCAGGTCTCTGATCCCCCCCCAAGCTGGGTTCCCCCAAGCCGCGCACCCGCGTGACTGCCCAGAGCTCCCGTCCCACCTCCTGCTA
GCCGAGCCCGCAGAACCCGCGCCTGCTCCGGCGCCCCAGCACGAGCCCGACGGCCTGCCCGCCGCCCTGCCCCCCCCCGGACTGCTCGC
CTCTGGTCCAGGCGTGGTGCTGTACCTGTGCCCCCGAGGCGCTGTGCGGGCAAACCTTCGCAAGAAGCACCAGCTGAAGATGCACCTGC
TCACCCACAGCAGCACCCACGGCCAGAGGCCCTTCAATGCCCCCTGGGTGGCTGCGGCTGGACCTTCACCACCTCTTACTAAGCTCAAG
AGGCACCTCCAGTCCCACGATAAACTGCCCCCCTTCGGCTGCCCTGCCGAGGGCTGTCGCAACAGCTTCACCACCGTGTACAACCTCAA
GGCGCACATGAAGGGCCATGAGCAGGAGAACTCGTTCAAATGTGAGGTGTGCGAGGAGAGCTTCCCCACGCAGGCCAAACTCGGCGCCC
ACCAGCGCAGCCACTTCGAACCCGAGAGGCCTTACCACTGCGCGTTTTCTCGCTGCAAGAACACATTTATCACACTCAGTGCTCTGTTT
TCCCATAACCGCCCCCATTTCAGCGAACACCAACTGTTTTCCTGCTCTTTCCCTGGCTGCAGCAAGCAATATGACAAGGCTTGTACGCT
GAAAATTCACCTGCGGAGTCACACCGGCGAGAGACCTTTCCTTTGTGACTTGATGGCTGTGGCTGGAACTTCACCAGCATGTCCAACAT
TCTTAAGGCACAAAAGGAAGCACGACGATGACCGGAGGTTCATGTGCCCTGTGGAAGGCTGTGGGAAATCTTTCACGAGGGCCGAACAT
CTGAAAGGCCACAGCATTACCCACCTGGGCACAAAGCCTTTCGTGTGTCCTGTGGCAGCCTGCTGTGCCAGGTTCTCTGCTCGCAGTAG
CCTCTACATTCACTCCAAGAAACACCTGCAGGATGTGGACACTTGGAAAAGCCGTTGCCCGATCTCCTCTTGTAATAAACTCTTCACAT
CCAAGCACAGCATGAAGACGCACATGGTTAAAGGCATTAAGGTGGGCCAGGATCTCTTAGCTCAGCTAGAAGCAGCAAATTCTCTCACA
CCCACCACTGAACTTACCAGCCAGAGACAGAATGATCTCACTGATGCAGACATAGTGTCTCTCTTCTCTGATGTACCTGACAGTACTTC
TGCTGCATTGCTGGACACAGCATTGGTGAACTCTGGAATCTTGACTATTGATGTGGCTTCTGTGAGCTCGACTCTGGCAGGCCACCTCC
CTGCTAATAATAATAATTCCGTAGCCCAGGCTGTGGACCCTCCGTCCTTGATGCCCACCAGCGAACCCTCCTCAACTCTCGATACCTCT
CTCTTTTTTGGAACGCCCGCCACTGGTTTTCAGCAGAGCTCCTTTAATATGGATGAGGTCTCAAGTGTAAGTGTGGGGCCATTGGCATC
TCTGGACTCTTTGGCCATGAAAAACTCCAGTCCAGAGCCTCAGCCTTTGACACCCAGCAGTAAGCTAACAGTGGACACAGATACTCTGA
CTCCTTCGAGCACCCTTTGTGAAAACAGTGTCTCACAACTACTGACACCAAAACCAGCGGAGTGCAACGTACATCCTAACTCTCACTTC
TTTGGACAGGAGGGAGAAACCCAGTTTGGATTCCCCAATGCAGCAGGAAACCATGGTTCTCAGAAAGAAAGAAATCTTATCACTGTGAC
TGGCAGCTCATTTTTGGTATGAAGCAACTCTATTCATTCCTTGCCATGTGCCTAACTTTTATTACAGTCAATTTTGAGGATATTCTCGA
CTAAATATTTAAGTGCAGTCATTTCTTTTTGGTTTGCAAAAAGAGCACAGCCCTGGACTATCAGTTTGGAGATCTAAATTCTGATCTTG
AGTCTCGAACTGACAAGTTGTGTGACCCTCAGCAAGTCACTTACCTATCTGAGCCTTAATTTCCTTATTTATAAAATTGTGGTGGTTTG
AACACATTGCTCATAAGGTCTTTTCAGTTTTGTTTTGTTTTGTTTTGTGATTTTGTGCTTTTTCTTGAAAATTTTCGGGCATTTTGCAA
TTATTATTGTTTGTACTTGTAATCAGAAGCGGTTTGAGCCCTGTAGCACTAAATAATGAAAGATTGAGGAACTGGTGTTTTACATTAAA
AATTTAATGGAAATTTTACACAGTACGAAAATCTAATGATAGAGCTCAAAAAAAAGAGCTAAA.AATAGGACTTGGTTCTTCTTAGCTG
TTTATCCTTCTAACCTTTTTTTTAAATGATGAAGGTATGTTTTTGTTGTGGAAAATACAAGGGCTTTTGTTATCACTCAGACCCAGAGT
GAAATGTTTGCTTTGTGGTTTTGAATAAGTTGGCCTTTAATAGTTATTTAGTCTCTCTTAGCCTCATTTTCTATCTGTAAAAAATGGTC
ATAGCAATGTGAACATATCTGTTACATCCTAGACTTATTTTTCTACCCCAGTAGGTTGTATTGAAGGAAAAATGTTATATGTGTTTCAG
CATGTTTTGGTGAATCTTCATTCCCTTTCCTGCCCCTTGTTTTCCCTCCTCAAAGGGGAGAAATTAGCACAAATTAGTATCAGGATTGT
GCAGGAAATAAACATTTCTGAACGTTAAGAAAGAGGAAAAGGAAGTTATTTCTTCAACAGATTAAGGATTTTTCTACACACAGTTCTTT
TGTGGCATTGGCCACATGTCCATTAGACCAATTTGATAGTATCTTCGGTCTGCATTCAAAGCCAGCTCATGCAATGACTATTCAGCCTA
TTCTTCCAACACAATCTGGACATAGACCAGAGGGAGATTTTTCTCCCCTCTGTGCCCTAGAGAGCTCATTGCTGGCTTACTCTTAGAGT
TGAAATGAGAGGGTTTTG
HUMAN SEQUENCE - mRNA

(SEQ ID NO: 23)

CTGCTCGCGGCCGCCACCGCCGGGCCCCGGCCGTCCCTCGCTCCCCTCCTGCCTCGAGAAGGGCAGGGCTTCTCAGAGGCTTGCCGGGA
AAAAAGAACGGAGGGAGGGATCGCGCTGAGTATAAAAGCCGGTTTTCGGGGCTTTATCTAACTCGCTGTAGTAATTCCAGCGAGAGGCA
GAGGCAGCGAGCGGGCGGCCGCCTAGGGTGGAAGAGCCCGGCGAGCAGAGCTGCGCTGCGOGCGTCCTGGGAAGGGAGATCCGGAGCCA
ATAGGGGGCTTCCCCTCTGGCCCAGCCCTCCCGCTTGATCCCCCAGCCCAGCGGTCCGCAACCCTTGCCGCATCCACGAAACTTTGCCC
ATAGCACCGGGCGGGCACTTTGCACTGGAACTTACAACACCCCAGCAAGGACGCGACTCTCCCCACGCGGGGAGGCTATTCTGCCCATT
TGGCGACACTTCCCCGCCGCTGCCAGGACCCGCTTCTCTGAAAGGCTCTCCTTGCAGCTGCTTAGACGCTGGATTTTTTTCGGGTAGTG
GAAAACCAGCAGCCTCCCGCGACGATGCCCCTCAACGTTAGCTTCACCAACAGGAACTATGACCTCGACTACGACTCCGTGCAGCCGTA
TTTCTACTGCGACGAGGAGGAGAACTTCTACCAGCAGCAGCAGCAGAGCGAGCTGCAGCCCCCGGCGCCCAGCGAGGATATCTGGAAGA
AATTCGAGCTGCTGCCCACCCCGCCCCTGTCCCCTACCCGCCGCTCCGGGCTCTGCTCGCCCTCCTACGTTGCGGTCACACCCTTCTCC
CTTCCGGGAGACAACGACGGCGGTGGCGGGAGCTTCTCCACGGCCGACCAGCTGGAGATGGTGACCGAGCTGCTGGGAGGAGACATGGT
GAACCAGAGTTTCATCTGCGACCCGGACGACGAGACCTTCATCAAAAACATCATCATCCAGGACTGTATGTGGAGCGGCTTCTCGGCCC
CCGCCAAGCTCGTCTCAGAGAAGCTGGCCTCCTACCAGGCTGCCCGCAAAGACAGCGGCAGCCCGAACCCCGCCCGCCCCCACAGCGTC
TGCTCCACCTCCAGCTTGTACCTGCAGGATCTGAGCGCCGCCGCCTCAGAGTGCATCGACCCCTCGGTGGTCTTCCCCTACCCTCTCAA
CGACAGCAGCTCGCCCAAGTCCTGCGCCTCGCAAGACTCCAGCGCCTTCTCTCCGTCCTCGGATTCTCTGCTCTCCTCGACAAACTCCT
CCCCGCAGGCCAGCCCCGAGCCCCTGGTGCTCCATGAGGAGACACCGCCCACCACCACCAGCGACTCTGAGGAGGAACAAGAAGATGAG
GAAGAAATCGATGTTGTTTCTGTGGAAAAGAGGCAGGCTCCTGGCAAAAGGTCAGAGTCTGGATCACCTTCTGCTGGAGGCCACAGCAA
ACCTCCTCACAGCCCACTGGTCCTCAACAGGTGCCACGTCTCCACACATCAGCACAACTACGCAGCGCCTCCCTCCACTCGGAAGGACT
ATCCTGCTGCCAAGAGGGTCAAGTTGGACAGTGTCAGAGTCCTGAGACAGATCAGCAACAACCGAAAATGCACCAGCCCCAGGTCCTCG
GACACCGAGGAGAATGTCAAGAGGCGAACACACAACGTCTTGGAGCGCCAGAGGAGGAACGAGCTAAAACGGAGCTTTTTTGCCCTGCG
TGACCAGATCCCGGAGTTGGAAAACAATGAAAAGGCCCCCAAGGTAGTTATCCTTAAAAAAGCCACAGCATACATCCTGTCCGTCCAAG
CAGAGGAGCAAAAGCTCATTTCTGAAGAGGACTTGTTGCGGAAACGACGAGAACAGTTGAAACACAAACTTGAACAGCTACGGAACTCT
TGTGCGTAAGGAAAAGTAAGGAAAACGATTCCTTCTAACAGAAATGTCCTGAGCAATCACCTATGAACTTGTTTCAAATGCATGATCAA
ATGCAACCTCACAACCTTGGCTGAGTCTTGAGACTGAAAGATTTAGCCATAATGTAAACTGCCTCAAATTGGACTTTGGGCATAAAAGA
ACTTTTTTATGCTTACCATCTTTTTTTTTTCTTTAACAGATTTGTATTTAAGAATTGTTTTTAAAAAATTTTAA
HUMAN SEQUENCE - CODING

(SEQ ID NO: 24)

ATCCCCCTCAACGTTAGCTTCACCAACAGGAACTATGACCTCGACTACGACTCGGTGCAGCCGTATTTCTACTGCGACGAGGAGGAGAA
CTTCTACCAGCAGCAGCAGCAGAGCGAGCTGCAGCCCCCGGCGCCCAGCGAGGATATCTGGAAGAAATTCGAGCTGCTGCCCACCCCGC
CCCTGTCCCCTAGCCGCCGCTCCGGGCTCTGCTCGCCCTCCTACGTTGCGGTCACACCCTTCTCCCTTCGGGGAGACAACGACGGCGGT
CCCGGCAGCTTCTCCACCGCCGACCAGCTGGAGATGGTGACCGAGCTGCTGGGAGGAGACATGGTGAACCAGAGTTTCATCTGCGACCC
GGACGACGAGACCTTCATCAAAAACATCATCATCCAGGACTGTATGTGCAGCGGCTTCTCGGCCGCCGCCAAGCTCGTCTCACAGAAGC
TGGCCTCCTACCAGGCTGCCCCCAAAGACAGCGGCAGCCCCAACCCCGCCCGCGGCCACAGCGTCTGCTCCACCTCCACCTTGTACCTC
CAGGATCTGAGCGCCGCCGCCTCAGAGTGCATCGACCCCTCGGTGGTCTTCCCCTACCCTCTCAACGACACCAGCTCGCCCAAGTCCTG
CGCCTCCCAAGACTCCAGCGCCTTCTCTCCGTCCTCGGATTCTCTGCTCTCCTCGACCGAGTCCTCCCCGCAGGCCAGCCCCGAGCCCC
CAAAACACCACGCTCCTCCCAAAAGGTCACAGTCTCCATCACCTTCTCCTGCAGCCCACAGCTAAACCTCCTCACAGCCCACTGGTCCT
CAAGAGGTGCCACGTCTCCACACATCAGCACAACTACGCAGCGCCTCCCTCCACTCGGAACGACTATCCTGCTGCCAAGAGGCTCAAGT
TGGACAGTGTCAGAGTCCTGAGACAGATCAGCAACAACCGAAAATGCACCAGCCCCAGGTCCTCGGACACCGAGGAGAATGTCAAGAGG
CGAACACACAACGTCTTGGAGCGCCAGAGGACCAACCAGCTAAAACGGAGCTTTTTTGCCCTGCCTGACCAGATCCCGGAGTTGCAAAA
CATGAAAGGCCCCCAAGGTACTTATCCTTAAAAAAGCCACAGCATACATCCTGTCCCTCCAAGCAGAGGAGCAAAAAAGCTCATTTCTG
AAGAGGACTTGTTGCGGAAACGACCAGAACAGTTGAAACACAAACTTGAACAGCTACGG1AACTCTTGTCCGTAA

TABLE 5
(mouse gene: Nfkb1; human gene NFKB1)

| | |
|---|---|
| Mouse genomic sequence | (SEQ ID NO: 25) |
| Mouse mRNA sequence | (SEQ ID NO: 26) |
| Mouse coding sequence | (SEQ ID NO: 27) |
| Human genomic sequence | (SEQ ID NO: 28) |
| Human mRNA sequence | (SEQ ID NO: 29) |
| Human coding sequence | (SEQ lID NO: 30) |

MOUSE SEQUENCE - GENOMIC

(SEQ ID NO: 25)

TCTTCTGAATATCATGTTTAGATTATGTAAGTATTCACCGGCTTTCTGTCACTATAAACCAATACCTGACTTGATTAATGAATAAAGAA
AAAAAGGTTTAGTTGATGAGTTTTGAAGGTTCCAGTCTACCAGTCTGTTACTGAAGACGTCCACCGTGATAGTATGTCATGGCCAGAGC
ACCTGGCAAAGCATAAACTACCTGCTCGTCAATCAGGAAGCAAATAGAAAAGAGAACTGGTTGCGGTCCCACAACATCCTTCCAGGTCA
TGTCTTAGTAGAGCACTTCTCCCAGGAGAAGCACCCTGTTGGCCTTGCCGTCAACACATGAACAGCTTGGGGACTCCTACTGAGGTAAC
AGTACCCATTCTGCATACATTCCCTGAGATTAACTGAGACTTGATTCATGGCCCGCTGTTTTGTCTGCCTTTGTGAGTGTTCTGCTTGC
AGCCCTACTAAACACAGATGGCCAATGTGGTATTCAGAGTAGTCGGCCTTTAAGAGGTTATGAGGTCATAGCACATACCCTGCCCCTGC
CATGAATAGGATTTTAAAGGGGGCTGATGAAGGAGGGTTGTCCCCTCTGACTCTTCCACCTTTACTGTGTGACAATAGAGTGATTGTTT
CCTCGTGACATTCCTCATTTGCGTGCTTTGACCTTGCACTTCTTGCCTCCAGAGTGATAAAAAATAAACTTATGTTTTTTATAAACTAC
CCAGACTCAAGTACTGTTTAACAGTACAGACATATGAAGACAGTTGGTCGTGTTTGTAGTCAGCACCGAAAGAGGAAAGTTGAAAACTT
CCACTCTACTTGTGGATTTCTGTCTCTTCCACAAATGTTTAGGTCTACTACTTTGTACTTCATGTATTTTGGAGACCTATTAATAAGTG
CATGCAGGTGGAGGTCATTATTTTTATAAGGGCTCAGTAGTGCATACATATAAGGTTCCAACACTCAGCATCTAGAGGCAGGAGGGTTG
CTGGGAGTTTGAGCCCAGCCAGAGCTATCTATAGAGACCCTGTCATAAAACAACAAAATTATTACTGCCCATTGATGACCTGGCCCCTT
TATTCTTATATATATATCCTTCTTTGCCCTTAGTAATATTCCACCCCCTGATCTTCTCTATTGGTGGTTATGATATAAACTACTTTGCT
GTGTTTAGTATTTGTATTATTAGCACAGTATCCCAGGCTTCTGCTTTTAAACACTTTGCGTCTTTCATGTTAAAAATAATTCATAGGAA
AAGCATTTTCTTGCGTCTTGCTTTTTCTGTACTGTTTACCATGAATGCTTTTTATAGCTGAGTTTATTTTATTTGCATTTAATATTGGA
CCTCTTCTTTATCCACATTTTGTCTTTCATTGTCATCTTTTAAATGAAATAAACGTAGCACTTGAAGAGTTCCATTTTATCTCCCGTCT
GGTAGTTCTGTAAAACGGTGGCTTTCTGTAGTAGGACTTTCTGCATCCTGCGTCTATAGCGAGTGAAGCGCTATGTACCTAATGTGGAA
GCTTTGATTTGTTCTCACCTCTCTGCTAGTGCTGGTAGACTTTTTTCCCTCCATGGGTTCAGAATACTCCATAATATATTGTGTTGTTC
AATGTGTTTGTTGTACAGTAAGTTTGAACATACTGCAAACCGAAAGCCCCATGTGCCCACACCTTTATCACCAGTAGTGTTTTCCTTGT
TTCTTAAAGGTCCAATTCTATCTCATTTCCCTTCACCACAAAGATCTGCTTTTAGGATGTCTTCTGTCACTTCTACGCGTTATGAATTC
TCTCAGCTCTGCTTTATTAGAAAGTGTCAGTTTACCTTTATTGTCAAACCATTCTGGGACAAAGCTACCTGCTTCTTTGTGGCTTCAAT
GTTTTTGTTGTTCATTATAGATATCATGATGTGAAGAGTTTGCATGTTTTTCTTTTATTCAGAGTTGAGTTTTGTCCTAGTAGGCCTTA
CTAAGATGCATTTAAGTCGCCCTTATTTTATGATGTGTTCTTGACTCTTATAACACGGTTTTCCCCTAGACTGCTATAAAGCACACATG
TTCAGAACGTTTACTGATGTCTCTTCAGCCCACGTGATTGCACCCTCAGTGTCTCTCAGCTCTGTATGGCGTCTGTAATCTTATCAGAG
CATTTATAGAACAACAGTGGCTTTCACTGAGCTTTTTGGGATCTCTGTTAGGATTTCATCAGAGTAAAGGTGGGCTGATTATAGATTTT
TGAGCCTTTGCACATATACCATTTCTCTGACCAATGAGATCACTGCTCTTTATTTGAACTCTGCTTCCTGTGGCCTGGATTAGAAAGTG
CCCTCAGTCAGAAGGTGAGCTTGGAGTTCACTTAAACTGTGCAGCCTGTATCTCTTGTTTAGTGTCTCAGGCTATTGTGTTTCATAATT
ACATATGAATTTGGCAAGATCAAATAGTGGTTGATTTATAATGACTGAAATTAGATTTGTGTGGATTTTTAAAGATTTTTTTTTTCAAG
ACAGGGTTTCTCTGTGTAGCCCTGGCTGTCCTGGAACCCACTCTGTAGACCAGGCTGATCTTGAACTCAGATCTACCTGCCTCTGCCTC
TGGAGTGCTAGGATTAAAGGCATGCTCCACCACTGCCCAGCTGTAGCATCCTATTAATGATTTTGAAAGACATTATTCCAGGGCATTTA
TATATTCAATATGTTGGCTAGCTAGCATTTCTCTACAGCTTTGTTTTGAAACTTTCTCATCATCCCAGAAGAGTCCCTTCAATGTGGTA
AATGATGGCTCCCTTCCCAGTCTTCACTCACTCCCCTGGCCTCTGGCAAGCAATGATGTGTTCCTCTTCCTACGAATGTGCTTGTTCTT
GGTATTCAATGTGAAGGGAATTGAGTATAGGTGAGCGACCCTTTCTGTCTGGTTTCAGTATAAATCATTTAGTCTACTGGTTTTGAAAT
ACAAAATCAAAACTCCCATTGCTGGCTGTAAGGCACACCATTTAAGGAGACTGTAAATATATAATGTATGATTAAGTTGCATAGAAACA
GTGAACCATAGTGCAAAACCACCAGGAAATGTTATAGACCTTATGGACTGAGGCGATAACAGTGGAAGAGAAAATACAGGTTTTATGGA
AGAAGTTGATTTTGAAGTACCATGTTGTGGAATACATAATATCTAAACAGGATAGAATCTCGAGTGTGAAGAGATGTACACACTGGAAA
GAAATGACATAGAGCTGTTACATGTGTCTGTGTACACATGCGTGTGTCCGTGTATGCAGTGAGCATTCACATGTGTAGAGGTCAAAGGA
CATTTTGGAAGCTGGTTCTTTCCTTCGAGCATGTAAGTTCCAAGTAAGGAACTTAGGTCCTTGGGCTTAGCACCAAGTGTTTTTACCTG
CAGAGCCCTCTTGTTGGCCAAGCAATGCATCTTTAAATGATTGATTTCCCTATAGTAAAGTGCTTGTGTTGGATCATGGTGATAGTTGT
GGTGCTTATATAATATATAATATAATATAATCATATGCTGACTAGTTGCTCTTTGGACTTCACTATGTAAAGCACCTGGCTGTGATCTG
AAAGATATTTAATCAGGAAGATACCCTGCCATGGGGGTGAGCGGTAGAAAAGCTGTTTCTGTCAGAGTGACGTAATAAGGTCTGCCCTA
GGGTGTGGCTATGGACATAGAAAGGAAGTGCACACACAAGAAACAGTGTGAGGAAAGAAGTGAGCGACAACAAGACTGTTCACTGCATT
GGTTGGAGAAGGCTACATTTGCAAGAATGAGACAGTCAGGAGTCGGGCTTGTTGTCGAGTAGTAAACTCTAGACATTACGGTTGATGGG
TTTAATGTATCATTAACTTGTTTTCTAATGGCATCGTTACCTAAACCTAACAGGGGCTCATTACCAAATACACCTTCAGAGCCTTAACA
GTTCTAATCATGTACTGATGTCACGGCGGTTGCCCTCCTATCTTGGATAGCTTGCGTTTTTTCTTTTGTTTTGTTTCTTTTTGCTCTTT
CCTTTTTTTAAAAAAAATTATTTATTTTTTCTTCAATTCAGAACTCAGTAGCCGATCGAGAAAACCCACTGCAAAGTCCACACCGTGAC
TAAGACTGTAATTAAGCTGTGATACTAAATTGCCAGGGTAATTAACCCATGCTCCACACGCCCTCTGTTCCCATAAACACTGACCAAGC
TCTATCGTGTAGGCTGCTCTCTTTCCATTTACTAACCGTTCTAAATTGGCTAGCCTTTAACTGCGGCCAGATATCATGAAGAACCACAT
AGAAACACACATATGCCTCATTTCTTGCGACCTCTGACTGTGTGTCCTCATTTGTCTGGCTGCTCTGTGGACGTCCTCAGCTGCTCTGC
CACCGAGGCCTCAGCCTGGGAAGTCTTGGCTGAGCTCTCCTGCGTTTCCACTCCACACGTGCGTTCTCATCCTCCACTGCGCTGGCCAG
GCCTTTCCCTTCCAGGAAACAAAATCCAGTCATTGAACCCCTTCTTGAAAGCCATCTTCATGACTCATCTCTGCATCCTCCTGTTGGTC
AGGGCCTGCCAAGGGCCTGACAACATTCAAGGGGCAGAGATGTCAATTTTATTGTCCCTGTGTTGTGAGGGATAGAAAAGGGCAGTGGC
CATTCTTTTGAGCCTACTACACCTATAAGCCCTGAGTTCACACTATGCCACTCTTGTGTAGTTTTGTTTCTTTGGAAACTGACAGGGTA
GGCAAACCTCTTTCTGATGGGTTGATGAAGGGCCAAAAACATCCAGGTAACATTTCAAACATGGTTTCCCTGTGATACAGACCATGGCA
GGCAAACCTCTTTCTGATGGGTTGATGAAGGGCCAAAAACATCCACGTAACATTTCAAACATGCTTTCCCTGTGATACAGACCATGGCA
TACACAAGTATCACAAATCTCTTCCTACACCACTGTGGATGACACACACACACACTCTCTCTCTCTTTCTCTCTCTCTCTCTCTCTCTC
TCTCTCTCTCACACACACACACACACACATGTTTTAATTTTTAGTTTAGCTAACTGTGGCTTTTGTGAGATATTCTCTCTCTCTCTCTC
TCTCTCTCTCTCTCTCTCTCTCTCTCTCTCTCTCTCTCTCTCTTTCTCTCTCTCTCTTTCTCTCTCTCCCTTCACGTTTGGATTGCCTT
GTAAGTAGCTCATCTGAAATTCATGCACTGTAAGGTCCTATTCTGTTCTCTGGGTTACTCTGTAGAAGAGTTCTATGGGCTCACCAGGT
ACTAGATGTAGGAACAGAAAGCACACTCGGGAGAAGGCATGTTGCAGAAACGAGAGAAACATGCTGAGACTCCTAATGTGCATCTCCCC
AGTCGAGACAACACAATTTGACTGGACAGTGTGTGCATCAAGGCTAAAGAGCGCTCAGGGAATATAGAGGGAGCCTCTTCCCCCTCCAG
TCACCGTGACAGCATGCTACTTCGTGTGTTGGCATGCACAAAATTGGCAGAACGCCTCAAGTGTGGAGAATTCACCATTATAACAAGAT
TATAGTAACCAAAAAATCACTCCACATCACGTAGAAGAGGGAGTTGGCAAAGGCGGCTGGTTTAAAGCAGCTCTTTGCAATCTTCTCTA
GAGGGTCTTATCTGAAACCAAACAAAAAACAACAGGAACAGCTTGAATGTTAGCAGTTATCCTGGCAGGCAGCACTCCAACCCAGCCCA
CCCCACCCAGCCCCCAGCACAGGTGTGTAACCCTTTAGTAGCCTACTTAATATGGCTGAAGAGATAGATGGCTTAACTAGTAAGAGTGC
TTACTGCTCTTGCAGAGCACTCAGCCTGGTTCCTAACTCCCACATGATCACTGACAACCTCCTGTCACGCTGGTTCCAGGGGATCTGGC
ACTCCCCTCCTTGCCTCTGCCAGTACCCTTACATTTGTGGTGCACAGTCATGCTTGCAGGCAAAACACTCAAGCCACATAAAATAAAAA
TTAATAAAACCTTGAAAAAAAGATCCTAGCTAGCAGTTAATATCATGAGTAGACTAGCTCCAGCCAGAACTTTTGAGAGGCTTGAAGCA
AAGATGTTTTTAAATGAATAATACTTCTGTCAAGAGGAAAATCACATCACCCATTACGCTAGTACAAGGGCCATAAGCTGTGCAGTTAG
AGAGGCCAGCTCTAGTCTGTCAGCCTGAGCAAATGACTCATTCTCTCTGAAGCTTGGCTCTTCAGCTGGACTCTACTTTCTGGGCTCAG
CATAGCAAGAGGGCAGCATACGTAAGCATTACCTCCGGTTCCTTAGTTATGTGGAAGCAGTCTGTCAAGCTTAGCTTTCCCCATACAAA
GGAGAGCACTTGTCCTGAGGCCAGAGCACTCACTGGCTTCTCCTCCTCAGCGTAACTGGCACCAGGGATTAGGTAAATTGTCCCTGGGT
TAATCCTGACCCTTCCAGGATCTTTGGATCACTTCTAGAAGATGCTAGGACCAGCCATCCAGTCCTGTGAGCCCAGAATACTTCAGAGA
ATAACTTCTAAACTCCCCCTCAGGGAAAAATACAAGTTATAGGGTAATGGCATGCAACCACTTCAGACTCAAATATGTTAGACTGTCAG
TAGCTATTGGCAGAAAATTTATCTGTCTTTGCTACAGTTCTATTGCTGTGAAGAGACATCATGACCAAGGCAACTCTTCTAAAAGAAAG
CCTTTAAATGGGCCTGGCGTACAATTGTAGACGTTTAGTCCATTATCATGATGGCAGAGAGCAGAGGACACAGGCAGGCACAGAATCAG
GAGCTGAGAGTTCTGCATCATGGTCTGTAGGCAGAGAAAGACAGACTCTGGACTTGGGATGGGCTTTTGAAACCTCAGTGCTCCTCCTC
AGTGACAGACTTCCTCTAACAAGGCCACACTTCGTAATCCTTTTAATCCTTTCAAATAGTGTCACTCCCTGGTGACTAAGCACACAAAT
ATATGAGCCTGTGTCTTTCTCAGGACAACGAAAAGTTTATCTTGCATCTTTCTTCCTATAATTTATCATGAGTGAGCTATTAGACTGTC
TGGTGAGTTAACATATGTTCTGTATCTCCCACTCACTAATAAGCTTCGCATGGATTAGCTAGCTGAGTGTTTCCCATGGGTCTGCCAGC
CCATTAGCCTGACACTCCATCATGAATGGGTTTCTTCGTCAGCTCTCAGATTTGCATTTGCAGTCCGTGCATGTATTGTTCTAGGTCAT
TGTCACGATGCTCCTGGTGAAATAATATTGAAAATAAAAGAAGTGGGAAACCAGGTTCTGTAAATCTCAGTGTGCTAGCATGCACAGAA
ACATTGATTTATTTTTAAAAGAAAGCTTTTAGAAATCAGCATTACTGAAAAAAAAAAAGTCCATTGCCAGCCTTTATCATTAGTGTGAC
ATACTCTCAGACAGGAGGACACACAGAAAGGGGGACGCTTCTGAAGAGCAGTCAGGAATAATTAGGAGTGTTGACGAATTCATACTTCA
TATTTTATTTAACACTCTCAGGGCATTTTAGGTTCAATCATTTAAATTTAGATTCAGCCCCCTTTTTATTCTAATACATGTCTAATGTG
TATTAGATAAATTTCCATGTCACTGTACAAATCTCCCCATTGTTATAGCAATTCCATAATATTTAACTTACAGTTGTAGAACAACAATG
CAAACAGTTAGATCACAGCACATCACAAACTCCTGAAAATCGACTTGCAGAATTTACGCCCTCTGAATATTTTACATGGTATACACTTT
ATGTGGCCATAGCCCTTACATTATGTTTTCTTCAAAAAATTCTCTATGGAAAAGAAACTTTAACATTTTATAATTTAATCTTTAGCAAT
TTATTGTGCGTGCAAGATACAAAACAAAAAATGTTGTGAAAAGTCTCTTTTCCCAGTATCTTTGCCCCATTGCTAACAATAACAGTACA
TTAAATGACACCTAAGTGACACCTGTTATGGTTACTAGAATTTTAAACCACCCTGCCACCCCACCCCCTCCCTGATTTCCAAGTGCACC
TTCTAGCTGGTGTCTGACCGCCTACTTTTCATAGTGCGCATTGTGGTTCTCTTTGGATTATGGTATTTTAATGGGTTTGGAAATTTAGA
CACCCTTTTCCCCCAAAGAAATCGAACAGAACAGAAACAGTTGTTGGTGTTGGTCGTGTTGTTGTTGGTGGTGCTTACAGTGAGTCAGC
CCTCGGCTGCCTATATCTCTGAACCTAGATGCTATATAGTTACTGACAGGAATTCTCCTGACTTTGTCCTTGATGGCATACAGCCATGC
CTTTAGAATTTCTCCACTCAGTCCAGCTGCTTCACCAAGATGCCCCTGTGTCAGATTGAGTTACTAGGAAGATTGCCACAGCAATGCAG
GAAACAGTTCGTTCAGCATTCATAGAATATGTCACATACTCAGGACTTGGCTGGTTTTGTTACATTGTGCTTTGTATGAAAACCACAAC
TCCCCGGATATTTGCTGAGACCTAGCTACATTTTCTGCGTCTCCTTATGTCGTGAGAAGATCCCTAAAGATTCCTGGTTGGGGTAGACA
AATCCATAAATGAGACCCAGGCTTGTGTCTCCCTTTGTGTCTCAAGGTCCTTCCTCAAAAGCGCAAAAGGAAAGTCGCGAAAAGGCTGA
GAAAGCAGGGGTACATAAGGGACTTTCCCACTCCTTTCTCATCCCCAGCTCCAAAGCTTAGTCGATATTTCAACATTTTAGGTATAAAC
TTGCTTTTGCAATCAATGCAAATATTACTCAGTGATTCATCCTTTCCAAGTGACTCTCGGTTTCTTTCTTTTGACATTTAATTTAAAAT
TAGAAATTAGCATTTATATCATGCTAGAGCTGCACTTACCTTTCAACGCCTCAGTAATCGCCAAGGCTTTATGCGTCAAQATTCTAAGG
CACCCGAGTCTTCCTTGAGCTTCTGTAGTACCTGACGGAGTTAGTTTTTAGCCACATATAAATCTAGAAACCGTAGGAGACGGTGCCAT
GATGACAAGGTGCCCAGTATGGTCGTTTGTCAGACATTTAAAGTGGAGCCGTCTTCATTAGGCCCTCATGTAGGGAATGGACCAGGAGG
ATACCCTTACGTCTGTGAAGTGCGGTTGGAGTTCAATCTTCCAAGTTGGAGTGAGACTTCTTTATGTTAAATTCCCCCACTCAAACTTC
TACAAATCATCCAATTTTAAAGAATGATTTTTAAGATGATAATATATTATATCATTTCCCCTTCCCCTGCCTCCAAGTTCTTTCATCTA
CCTCTCTCTCAAAACTTCATGACCTCTTTTTCTTTAATTGATGTATCTTTTTCTAAGTACAAAAAAAAAAAAATCAATCTGCTCCATCC
ATCTAATGTTACTTGTATGAATGTTTCCATTGGCATTGAGTAACCAATCAGTGTACATTTTTGGGAAACACAATTTCTCATGCTCTCAG
CTTTCCCTATGTGCCTGTAGTTCTTAGTCTAGGGTTGAGACCTCCTGAGCAATCTCCCTTTCACATTGCCTGTCTGTTGGTCCCATCCT
TGTTCAGGTACACACAGAATCAATGACATAAAGGTACTAAGTGGGTTTGGACAGTTTAATAAGGCAATAATGTCACCTCCACCTTAGAA
GATTGTCTTTGGATTGTTAGGAAAGAATATCATCACCCAGGTTAATGGAAAACCTAGAGTCATAAGGATAGTGTAAAACATTGTTTGTG
GAAATTAATTACTATCCAAAGTCACATACCAGACAGTGCCAGAACCCTGCTATTTTCAATGATGTCATACCCCTTTACTGCATCAAGTC
AAAATTCCCCTGTTTTAACTTTTTTGTCTGAATAAATATGTTTAQTTGCAGCTGCTTAGACACTTTTGTTTTGTTTGTTTCATTTTGTT
TTAAGACAAGAATCTTACTGTGTAGCCCTCGAACTCACCATGTAGACCAGGCTAACCTCTACCACAAAGACATCTACTTGCCTCTCCCT
CCCAATGCTGAGATTAAGGCGTGTGCCACTACACCCAGCTCGTCTTTTCTTTTTAAGTATGTATCAATAAATTGTGTGTTTGTGTGTGC
CTGCATAAGTTTATGTATACTTTATGTGTGCAGGTACCCTCACAGCCCAGAAGAGGCTGTCAGAGCCCTTGTAACTGGAGTTTGACGGT
CGTGAGCTGCGCATGTAAGTACTGGGAACTGAATCAGGGCCTTCTGCACGCTTAACTGTTGAGCCAGCTCTCCAGCTTGTGTTTAAACA
TTTTCAAGTGTTTCTTGTTTTAAACAACCCCACTTCCTTGCACACACCTTTGTGAAGTTGTCCCAAAGGACTTAGACCTCCTACTTTTC
CTTTAGTTGCTCCTCGCCCTCCTCCATCTGCCCCTGGCAGTTCTCCAGAGCCTCCCGTCCATCCCATTAGTCACGTCCTGTTATAGCAC
ACACCCTTTCACTGGAAATGAAGCCTCAAGTTATGCCAATGTCCCGAGATGTTTTTATTAGTGTAACATAGCCTTTTGATGGAGGCTAC
ACAGTACATTGGATTGAAAACTAACTAGCCTCCATTCTTCCATCAGCCTATCTTCAAGCTCCAAGGTGGCCACAACAGCCCCATCCTTT
GATACACAGTCTGTTCCCTCATGTCCTAGAAGCCACGCCTTTTCTACATTTGGGTTTTATTCAGCATCCAACGCAGTTTTTTAAAAAAA
GATTTATTTATTTATTTAATGTATGTGAGTACACTGTAGCTGTACAGATGCTTGTGAGCCTTCATGTAGTTGTTGGCAATTGAATTTTT
ACGACCTCTCCTCACTCTGGTCGGCCCTGCTCACTCCAGCCCAAAGATTTACTATTATACATAAGTACACTGTAGCTCACTTCAGACGC
ATCAGAACACCCCATCACATCTCATTACAGGTGCGTGTCACCCACCATGTGGTTGCTCCCATTTCAACTCAGGGCCTTCAGAAGAGCAG
TCAGTGCTCTTACGCGATGAGCCATCTCACCAGCCCAGAATGTGATTTTCACGTGCACATACATGGTTCTATCTGTTTATCACGGTCTC
TGGCTGCCTGAAGGCCGCTCAGGCTGCTTATATTAATAGCTTGCTAACTTCAGTAAGTTTGAGACTTTCAGGTTTGATGTTTTCACTGG
TTATTTTTTTAGTGCACTCTTTAGAGTTACAGAATTTTCATTGCACTTTGACAAAGGCAGAATCACTCTCACCAGTCCTCACAAGAGGG
CAGGGGGTGTCGTTCTATGCCCACTCAAGTCTTATGTAAAATGGTATCAGCACTTCCTCAGCCTCTCAACCGAGTCTAGACAGTCCTAG
GACCATCTCAGAGTTCATGACAAAAAAAATGCACAAAGTAGAAGTGGCCACAGGGTTCCATCTGGACCTGATTTTTACCCAGAGGTACC
ACCAGCTGTATCAAACCGAGGACCCTGAGCTTAGCCAAAGGGTTTAGATCCCGTGCTCTCCCTACAATCAGAGGTACATGCCAAGGACG
TACAGCACGCATGATGCCCTCCTACACACCTCTTTTTTGCACACATTTGTGGTCCCTGTAGAAATACAGCCTAGCCCTGCATATGTGCC
AGAGTTGTAGGATCTTTAAGATTCACTCCAGTGCTACAGCTTTCATTTCTTGTTCTCTTTTCTACAGCTTCACCATGGCAGACGATGAT
CCCTACCGAACTGCGCAACTAAGTCCTCAGCTCACTACCAACTTTGTTTCAACGCGTTCTTATCCCAAACACACAGAGGCAACGCTAGT
AACTCACTATCAAGCAAACCCTAAATTAGTAAACACTACTCTTAATTTATTTTCAGTTTTTCTGTTCTTCCAGAGTTCGAGAGGGCCCT
TTCTTATGTATTTTTGCATAAAACCGTATATACTACATGCCAGAAAGTAGCCACTGAGTCAGTTGTGGTGTTGAAGGTCTGACCCCAGT
ATTTCATATGGTGACTGGTATATTAAAAAAATTGCTATTCAGTGAGATTTCTAGGGCTGCTATTAACATCGCTACAAACCTGGTGACTT
GACACAATAGAAGTGTATCCTTTCATAGGTCTGGAGGCAAGAACTCTCAATTTGGGGCTGCCACAATAGTTCCCACCTCCTACCACTGC
TGGGTGTGTTTAGAGATCCTTGGCATCACACCAGCCACACTCTGTTGTTGTGTGGCTTTCCTCCACGTCTTCACACATACCATCTCCTC
TGTCTTAAAAACACATCACTTTTTGGATGTAGGATCCATCTCTGTCTAGTGTGAATTCATCTTAACATATATAAAGCCAAAGGCTGCCC
TGTCCAAATAAGTCACATGAAGGAGCAGCACTTATGAATTCTGAATATATTTTTTTTCAAAGGTACTGTAGTTAAGCCCACTGCAGTCT
GTGGGACTCAGTGTGCTTTGACAGCGGAGCAGTGTTTATCATCACGTCATTGTCGCTGTCATCATAGGTGTGACAGCAGCCATGACACA
GACTGACACGAGGATATCTGTATCTTCACTTTCCTTCTACTTTTTTCTCTTAAAAAATCTAAACCCATAGAAGTGTTAGACAGTCTGAT
GATACTTCCATTTCTTTCTCTTCTCCCCATAGAGAATATAGCCACCATAGAACTGATTCTTGAAAGCAGTTTCTAGCTGCTAGCATGAT
TACAGTGGCAATATTAAAAGTGGACTCTAATGGTCTATTAAATTATTTAAAATACTGGTATCTCTTTGCTCCTCAGCTATCTACTGAGA
CTTTATAGAAGACTTTATGCCCACAACATTAGCATCCTAGAGACGAAGGCACAGGTGATATGCTCACTGGGCTCAGGACTGCATCCCGG
CTGCAGTAACGGAACCACACTTTACACAGTCTGTGGATGTTGCCATGGTTCACTGAGAGACAGAAGCAGTTGGAGGGATTGCTGTGTGT
TCCTTTAGGGAAAGAGCAGGTTAAGGATGACTTTGCAAAGGAGGACTAAGCTGAGCTTTGAAAGCTACAACCAGTGCAGGGGCACTATT
TACCAAGGTAGCCCTGCCACAACATTCATTACTACTTCACATTTAAATTTTGGTGGTAAACACCCGAGACTACCATTTCCCAAATTTAT
GCTCGCAAAGAGTCAGCCAAAAATAAAACTGAAAATGAAATATTTGGGGGAGACCTGGTGTTTCTATAGTCACACAGCCCCACACCCCC
AGTCAGAGCTGCCATGGCCTGCACCGTAGTGACAGCTGCAGTGGCCTCAGACCTGCAGTCAGAGCCGCAGTGGCCCTCTTCTTCTCACC
CAGCGCGCGGAGTGAGCATGCACCATCCCCTCCTGACCATGTTATAAAGATGGTGGTACTGGGTAAACCTAAGGATGTTTTCTTTTATG
GCAGCATAAAAGACACCTGGCAACCATACGCTCAAACCCCTTGTTTAACCTTTCACTTTTAATGGGCCTCCTCTAACACAGGCCAGCCC
TCACCACCATGAGAGCTGTCTGCTTCGTTTCAAACCATGCTTTTCTGTCTGTACACAGGAAATCTTAGCAACCAGAGATGGTCTTTTAT
GCCACAAAAGACTAAGACCTGCCAAGGACAGTCATTTTGAAACTGTCTCATGTGGGCAGCAGTTCTGAGCTCATTAGCAGGGTGGCCAT
GTTCTGATAGTTCAGTTGTGGGGGAAGGGGAGGCCCTTGGAGGCTTTTAACTTTTTGTCTTTGGGTTGGTTTTTTCTTCCTGTCTTAAG
CTTTAAGTCTATCTCTTCTAAAGTTTGCGATTTTTGACGCACGTGTCCTCCGTGTTTTAGCCACACTGGAGTTTGTTCTCCACCTGTCA
CCAAGCAAACACCACATGCCGCCTTCAACAGGACGTCTGTCTTAGTGGGCTTCTCCCCCAGCCAGATCACTGCAGACAGAGGCGGAACC
ACAGCAAAGGACTTCTTTGTGAGGTCGTTCTCTTTGCTCAGAGATGCAGGCCTGGAGCTTATTATAAAACCTAGTTTCCCATTCAGTGT
GTCTTTCATGGTTAATAGCGAATGGCACAGATGCGCTTTCCTTTTAACGATTGAAATATAACACACTCTTTGCTCCTCAGTTGAAAATT
TGCATCCCTCAAAAGTGGATTTGAAGTGACTCGCATTTAAACATCACATTTTAGGGGCCACTAAGATTTTTCAGACAGCAAAGTGCTTA
CTAAGTTAGCTTGACTTTGACTTCCTCCACTACCCACACATAAGCACACTCATCTTACACACACACACATATCCATCTCATGTTAAACA
CACATACTACACATACATATGCACTCTTACACTATGCACACATACACACCTTACACAAACATATATCTCCATACACTCAAACACACACA
CTCACATACTCCCACTACACATACATACACTTTCACACTATACACACATACATGCACACACACTCACACTACATATACATACATACTTT
CATACTACACATGCATACACACATACTACGCATACATATACACTCTCATACCACACACACATACACTCACACTACACACACACATACAC
ATTCTCACACTACACACACAAATGTAATAAACATGCAAGCATTACTCTTCTTATTCTTGATTTAACTAGACATAGTAGGATTTGAGGTA
CCCAAGAATATCTAGTGATTTACTAATAAGGACAAAAAATCCCAAAAACCAAAAACCAAAAAAAAAAAGAAACAAAGAAAAAAACCGTC
TAGCTTGCAAGCTCACCAGAGAAAGTAAAAGAAATAATTTGGTTTCTATGTTGTCCATAAACATTTCAACAAGTCACTTCTGGTGAGTG
AGACAGATAGTAAGCTCACACATACAAATGTGATTTTCTAAATGAGCTAACTCGGAAAATTCCTTTTGGAGTAATTATTAGTCATAACT
TACCAAGGTAGAGCTTAATTCTATCAAAACGTTATATGTTCACTTTTTCAGTTTTCTTAGTTGTGTGTTGTTTCTGTAACAAGTTACCA
TAGTATTTGAAACAACACAAATATCTTCTAAAGATTAGAATAAAAATGTCAGAGTCGAGGCATCAACAGGGCTACACTCCTAGCAGCGT
TTATTTCTTGTCTTCCAAGCTCTAGGGGCTGCCTGTAAGTCCTGCTATCATCCCCCAGTCCTGCCTCTGTAGGCCTGGGTGAGTCCTGC
TACCATCCCTCACTCCTCCTTCTGTAGGCACACTGCACTTTGATTTCTCACTCTCCTAAGTAGCTCACTTGGATATTCCAGGATAATCC
TGTCATTTAATTGTCCTACATTGATGGCACATGCAATATATCTTTTGCCACAGAAAGCATAGGTTCTCGGGAATAGGAAGGTTCTGTGG
AGTAGCATGTAGACATCTTTAGAGCCATTGTTCAGTGTGCCTTCATCTACTAGATTCAGGTATTTATATTGTAATAGAAAATAGAGCTG
GGCAGTGGTGGCACATGCCTTTAATCTCAGCACTTGGGAGGTAGAGGCAGGTGGATTTCTGAGTTCGAGGCCAGCCTGGTCTACAAAGT
GAGTTCCAGGACAGCCAGGGCTACACAGAGAGACCCTGTCTCGATAAAAAAAAAAGAAAGAAACAAACAAACAAAGAAAGAAAGAAAGA
AAGAAAGAAAGAAAGAAAGAAGACGACGACCACGACGACCACGACGACGAAAAGAAAAAGAAAATAGAGGCGGGAGAAAGTGAGCCCTC
TGCTCCATCATGTTCTCAGTTGTATTGCCAGTCATTTTGTAAGTTGTGTAACTCCTGCCTGTAATTCTTTCAAGGCCTTGAAGTTCTGT
AGCTCAGTAAGTAATCTATTTATCATCAGAACAGAAGCATACATATCTAAGATCATGTCCACGTATGTCCTGTCTGTCTGTACGTGTAC
ATTCACAAGTGTGTGGAGGCTCAAGGTGGCTCCCCATCTTTGATTTTGAGAAACGGTGTCTAACCTAACCTACAGCTCCTGGATTCAGC
TAAGACCTCCAGCCAGCAAATCTTCATGTCTCCACCTTCCTGTAGCTGGGACTGCAGACAACTGCCGCCAGCCCAGCTTTTCACTGGGG
TACTGGGGCTCTGGACTCAGGTTCTTGTGCTCTACAGCAAGCACCTTATCAACTCGGCCATCTCCCCACCCTCTGCGATGTAATGAACT
CAACTTCCTAAGAGCGTGCGAGATTCACATCACTTACTCTACACATTTTTCTGAAGGGTCCAGCATGCCTGAAGCTCTCTTGGCTCCCA
AGATGCACAGCTTGCGGGATCCACCAGGCCCTTGTCTTTAGGGACCCCATAGCCATTATTCTCCTCAAATTTCTTTGTTACATAACTTA
CACACACAGCCCCCTAAGAACGTCCCAGAAAGGCACGTTAAAATTTTGATTTCACGAATCCTATTTTTGCGTTTTTATTTCTAGTTTTC
AATCATCTAATTAGTTTTCTGGCTTTGTGTATTACTCCAAGAAGAGAAATTAAAATATTTGTATTTTCTTCTTTACAGATCTTTCATTT
GAACACTGCTTTGACTCACTCAATATTTAATGCAGAATTATATTCACCACAAATACCACTGTCAACACGTAAGAAAAACACCCCCTTTT
CCTGTTGTTTTGCTTTCCCTTAAAAATATTTGGGCTCAGCACTCACGGAGATGTGGATCAACCAAACTAAGTATATATGAAATTACTAT
AAGGAAATCTGTTGCTTTGTCGCTCCTTGGGACCTACCCCAGCCCCTCTGCAACAGCCACAAGCACTTTTAACCACAGAACCATCTCTT
TAGCCACAACAGTCTCTTTTAAAATTCAACAATATTCATACGAAGGAAGTCGTGATCCGTCTTTTAAAACCACCCTTTCAGTATGCTTT
CTGCAAATCTGTCTCACAAAAATGCTCCCGCGTGCGCGCACACACATCCTTTTAGCAAGTGTGGTTTCTGGTCTCTGTTAGAATACCCT
GAAGCCCTGTCTTCACCCCCTGTAACCACTTTCCTCTTCTCCGTATCTCATTACCCATTACTGCTCCTTTTTCCTTTACCGCAGCTCAC
AGAGAGCCAGTCTTAGCCCCTCAGTTCTGTATCTGAGGGCCAGTCTTGTCAGCCAGGACACGAGGCTCTGGCGAAACAGTAGGAAAGTA
ACCTTTATATTCTGACCAGACCAAGCGTACAGAACTAATGCTCCTGACATGACTCCTAATTAACGAACCCACAAACTGTTTAACCTCCC
TTTATGCTATAGCGCACGGCCTTCCTTTAGACTCATTATTCACTCGCATGTGTGTACCCTTGGCCTCATGGCTCTTTTGCTGCTTTGAT
CACCTGTTCCAGCTAACCCGTCTTCAGCAGCCACTTGTTAGTTGGTGGACATGCGTATGGAAGTGATTGTCCATTACTGAGGTGCACAG
CACGTCCTTTGTCTGCTCCGGATTTCTGTGTCTCCTGATGTAGTTTTTATCATTCTCATTTAAAATCACTATGGTTTTGCCAGCGCCTG
CCTTACGGAAAGTTTTCTACAGACGTGGGAGGAAGGAGAGTTACTATGTCACCCTCCTCACTGGTGCCCAGAAGTTACATCACCCAGCC
CTGTCTGTCCTTACCCCAAATAACCACCACAGTTTATCTGTAATCTTAAGCTGTCCAACACGCTCTAGGTCCCCTTCTTGTCACCAAGT
AAATGCAGGGGAGGCAACCGTGTTTCCTCTCAACTTCTAAACTCTGAGACCCCAACAGCTTCCTTTAGCATTTCTGCTTTATTGAAAAT
GTTGCAGACTCGGTTCTGCCCTCAGGGAAAGCACAGGTTTCTAGAAAATGCCTCTAATTAGTTAGAATACTAAACAGCATCCTGCAGGC
AGATGATATTCTAATGTGTTCTCCTTTTCCTTTGGTTTATGCACTTACAAATAAAGTTATCCAAAGAATCATCTCAGAAAGTCGTTGGT
GTTCATATCATTGCGCTTCGGGAATCTTCTTTTCCATACTGTTAGAGAATCCTTCAGAATTCCATGCAACATCCATTCTTTTCCTGTTG
TAATCGCTCCTTGCTTCCACTACCTCTCCGGCTCTGTTTCAGTGCGTTGTCCTGTTTTTATAGTCCAAGATGGATGGGACATTTTGTTC
AGATTTTAAAGGACTAGAAAGGCTATCTGTCACAGTGTTTGCTTGTAATTTTCATGTGGATTTTTTTTCAATCTTCTCAACTTCTTAAA
ATCTCAACGTATTACTATTTCTAACCCTGTGTTGGGAACTCTCTAACAAGGGACAGCTGATAGGTCTGTGTCATTTTGCTGTGATTTAA
TTTTCTTTTTACAATGCAACATCATTTTTTTTCTGGCACAGAAATGTAGCCAACAGAATATTAGCCTTAAAACAAGTATCTGTGTATCA
GCTCCAGTGTTCTACTTCTCTTACCCTTGGGTGTGCTCTTCTAAGGGTAATGATTGCCCCCTCTCCGCATGGAACTTCACATATGTAAC
GAAAACCTAAAACCAATCCTGTGAGTGTTTCAGACGGGCTGTTAAGAAGGAACAGACTTGGAGCCTAACACGAGGATGTCTCCTCTGCC
TCCATATTTCAGCAGCATCTTATCCAAATGCTACCACCTTCCTGGCCTGGCTTCTCAGTAGGCTCTTTTGGATGCCTAGAGGAAATGTA
TATTTTTAATATTGTTATACAAGCTGTCTGATATGCCTAGCATGAAATCTTTTTCTAATCAGTTTTATTTCCTCATAAAAQTTAAATTC
GAGCAATTTTCTTTTTCAAATTTAAAGTTAACAGTTACCTTTCTAAAAGTTTAAAGTGTCAAGAGATGAATCCCATGAGACAGAAGAAG
GTAATTTGCTACATTACTGACCCCAGTAACCACCCATCTGTCTTTCTTATTTGGCTTCAAATTAGGTTTCCAATTGCTTTCCTGGTACA
GTGTTTACGCTTCCGTGTTATCTGATCAGTCATAATATTGAGAAGGTAAAATCTATTCTCTTTGAAGTTACAGTTTATTAAAAATTACA
ACCTGAACTTTTATCTCACCTCAAATTTACATATGCAAAATAAGGCTTAAGCTTCAGCCGAGAAAGTGAATTTTCAAATGACCACAGAA
GTGTTCTTCTAGACTGTCTTCAGCTTCAAGTCTTAATTTTCACTCAAACAACTATAAAATGTCGAATATGTTTGTGTATTACACAGCTA
TTGGTTATCATACTGGGACATAGAAGCCATCTGACCCCCTTATGGAATTTTTTATCATAACATTTATATTTATTTATTTTGAAATTTCA
AGTCACATATCATTGGTATATAAATTGAGCCTTTAGGTCCTTTTCATAGTAATTAATTGGCATGCATTTATCTTCAATTACATTATTTT
TGGTATTTTATCATAACTCTGGGCTGTTTTAGAACAGAGTTCTTATGAGCACACACTGTTAGATACTTTATTATATCTGCATCAAAAGC
TGTGAGTAAGATGCCAGGGTAGTGGTCCATGCTTTTAATCCCAGCACTTGCCAACAAAATCTCTCTCAGTTTGAGGCCAACTTTGTCTA
CATAGTGAGCTCCAGGCGAGCCAAGGATACATGGTGAGTCTCTGTCTAACAAAACAAAACAAGCCATGGAGAAGCAAGAAAAGTTCTTC
AGGTCTCACAGAGGGCAAGGAAGAAGCCAGTTAGGCAACAGTGGCTGTCCCTTGGCTTGAAGTAAGGGAAAGACTACATTTCAGGAGCA
GTGTTAGGAGAGGCAGGTGGCCCAGGATCTCCCTTCTGTGGGTTTCTAGCTTCAGTTCTTCATTTACTCCTGGGCAGTTCTCTTTGAGG
CAGGAAAGTAGTCTGCAGGTGAAGGCCACCTGACACCACAGCACTCAGAGGTGCTCCCTGCACTCAGCTGTATTGTGCCCTAAATGTTT
GTTGACACATGAGTCTGAGGACGGCTCCCATCACAATCTCTGTCAGCATTCAGACCTTTTGTTCAGTGCCAGTGGTGTACCATTACCAT
TCAGAAAAAGCCACTTATTTACCGTTAGTCACTTAAACTATAAAACTANNNNNNNNNNNNNNNNNNNNATCAGACACTCTCATCTGGCT
TCCTTGCCCACTAAGCAAAGCACTTATGAGCAAAAAATAAATATTTTTTAAAACATTTATTTAATATTTCAAATGATAACACAGATCAG
TAGAACACACTTCACCTATAAAAATTGATTTCAAATCTCTCTTTAAAGCATAACTTCAGCATTTGTCTTGCTTTGTATTTCATACAAGA
TTCCAGAAGTCTCACAGTTTTCTTTTGGTTTTCTTGTGTTTTCCTTGTTTTTAACAGATGGCCCATACCTTCAAATATTAGAGCAACCA
AAACACCTAACCCTAAAGGGGTCGGGTGTTTGGTAATATTACATCTCATGGTAAGGCTCGGTCTCTGAGTAATAGTTAAATCTGATGAC
ACAGGTAAGGGTTTTATCTGAATAATCGGCAGCCTGCCGTGTTATGCAATAACTTCCTGTGTCTACATTTTTCTTACAAATCCCTTTGA
AACATCAGTTAATTATAAATCCTTGGTAGTCTGTCCTTTTTCCAGCTTAGCTCATCAGCTGCTTCCAGGGAAAGCACTAAAGAGTCTCA
TTCATGAAGAAATACTCACACACACACAGCTTCACACCCACTCTGACCCTCTATTGCTCATGGCCATGCCCACAGCGTACCCCTCCTGC
CTTCATAAGCTCTGGCCTTTTCTGTGCATAGAATGTGTAGCTTAAGGCAGTAAACGCACTGGGAGACATTAGAGTGGACTGGGAGATCT
CCCCTCCACCCCTCTGCCTTTAATCCCAGCCTTTTTGAAGCAAGGAGAGGATTTTTTTGTTTCGTTGTTGTTGTTGTTTTTGTTTGTCT
TTTTGTCTGGCTCTTTCGCTTTCTACTAAATATTGTGTTCACCAGCTGAAGATTAAAATAGCCTCAAAACTGCAAATATGCTCATTACA
AGTTGCATGGCTCTAGTACTGTAAGAGAGAAGAGAATCTTCAATATCTGCCGACAGAACATTTCAGTCATTATGGGATGGAAGTTATGG
TATAGCCTCCCACAGAGTGAGAACCAAGTCACTAGATCTCTGCGTGGATATGCTCATCACAGCCGTGGCGCTGAAGGACTGTATACCTC
GCCTACCGAGTTGTGTCACCCAATGTCTTTTCTAGGCTGTTCCTTCTTGTAACTCAGGAGAGTGGGTTAACACATTGTGTTGTATGACA
CTCAATGCTAAGAAGTGAATGCTGACAGCATTCGGTGCTGTGCTGTAGAATTCTCATGTGGTCACCTGTTAAATAGGAGAAACATCTTA
GGGATGTCCATGCTACTTTGTAAAGCTGAAATCAGATGGAAATGTTAATTATAAAGACAGCAATGTCACATCGAACTTTGCGGCATTGT
TTCGTTCCTTATAGGCGGCCTCTTAACACACACACACACACACACACACACACACACACACACACACACGCACACACACATACTTATAC
ACATGTACACCCTCCCTGGCCTGCAGATAACACCTGCCCTTGCTTTATCTCCACGTCCATCTTTGTAAAGATGATTTCTCACATAAAGT
CGCTGTTCTCGGAGCTCTTCAGTAGAGTTGTGTTCTGTGAAATTCTGCCCAGTAATCTGGAATCCTGGAAAATGTCCTAGCCTGGAAGA
TTTGCTCAATTCATAAAACTGATGAGTCCAAAATGTACCTACACATCAAATAGATTTTTCAGATCCTTTAGAAAATTTTAAATAATTTA
TTTAATTTTATTTTGTGCGCATTGGTGTGATGGCGTCACACCCCTTGCAACTGCAATTACAGACAGTTCTGAGCTATGACGTGGGTGCT
GCGAATTGACCGCAGGTTCCTTGGGAGAGCGGACAGTGCTCTGCTGAAACATTTCTCTAGCTCCCCTTTTTAACAATATTAAAATTGCA
TTGATTTATTTTTTAATGTGTTGCTCTCTTTGCCCCCACCCCGTGTGTGTGTGTGTGTGTGTGTGTGTGTTATTATGAACCCCAAAGTC
ATAGGTATTTATGATGTGTTTGAACAGCTAGTATAGTTCCAGAATTTAAAATTTAATTCATTTTCTGTGAATATATAAAATCATATATG
TATAAAACATCATGTTTGATTGCCAGTCCGATTTTATTTTGGTATCTGGCTTTAGTGAAAGTAAGAAGGTTGTATGTAAGCCAGGCTTT
TTCACTCCATGTATGGAGTCTCTCTGAGGTTAATGACTCATTTGAAGTAAATGGCGTTCCTGAGATGAACAGCGATAGATGGTGAACGT
TTAATTTCAGAATACTAGAATTAGAAATGTGTCCAGAGCGTGCATTTTAAGACTTCCTAATTTTAGGTTGTTATTTAATTTAAAACATT
AGTTTATTTCTGAGTCAAAAAATGTCTGTAGTCATTGCTGGCTTCAAACTGGTGCCAGTTTTCCTACCTCCCCTCTCCAACTGATAACA
TGACAGCTCTGACCCACCATGCCCAGCTCAAAGGCATCGATGCTCCAGCCGAACAGCGCAATACACATTACAACCTTCAGAATAAATGC
TGCCTTGGATAGGACGTTCAATATTCTTATTCGACCGACAATAATACACACTCTCACACCCTGTCCAATAAATCATGCTGGCTACAGGC
TTAAACGTCACTCCCCATAAGGTATGAGAGGTACAGGATACAGATTTTTCCAATGCCACAAAGAAATTTTGTAATTAAAGATAAGGAAC
ACTATGTTAAGTTTTTGAQTTAGAAATCTTCTTTTTCTTCGAGGCCTTTTCGGCACAGATGATCAAAATCAACCAAACCACAGACTACA
TGTCTTTAGACTGTCCAGTTCCGTGGCATGTTTCTCACTGTTAACTGTTTATTCATGTTGTGTTGTGTTGTGTTGTGCTGTGTGAGTTT
TCTACTGCTTTCTTTGGAGGCAGGGTCTCAGCAGTGTAGCCTTGGCTCGCCCCAAACTCACACAGCTCCATTTACGTGCCTCTGCCTCA
CAAGCACTGGGGTAAGATGTGTGGCAGTTATAATCTGAAGGTGTGTTTGAATTTTCTCAGTTAAGTGGATCCCCATCCTAACGGCAGCC
CATTTGGGGCTCTTCGTGTTATACATGCTATTATTGTATAGTTTCGGGGTGATTGAGTTCAAGCGTTATCCAGACTCCATCCCAGTAAT
ATTAATTTTTACAAACTTTATGATCTTATTCCTTGAAGTGCTCACAAGACTGCTAGACTCACACTCTCGTAGCATACAGCATGTCTGTA
ACACAGATAACACTTAATCCTCCAGAGACTTGAGAAGAAGCACCNAAGAGAAGGGGGATGTGGGGGGGGAGCACCCTCTCAGTCGAGGA
GGAGGAGGAATGGCCTCACGAACTTCCCGGGGTGATGTAAATAAATAAAATAATTCTTAAAAAGACACCTTTATGGTCTGTCATCACAG
CAGATAACGAACTCCTTTAAGTAATTAGCATAACTTTTACTCATGCCACAGCGGAGAGCTTAAGCACCAGCCATTGGTAACGAGAGGCT
CCAGTTGAATAAAGTGCTAACGTATATCCAAAGGCTTAAACTCTTCATCACTCCTGTGTTGAAGGTGGGTGCCTGGCTTCTCCGCCACT
GCTCACTGCATCATTTTCATAGTGGCTGTTAGAAACCATTTCTCCGTCTGCAGAGTTCTCTTCCACAGTTTTGCATGTTTAGAGACGTG
TGTGTACATCTGTCATTCTTCCGTTCAGTAAATACCCCTCTGTTCACCTCTGCAGAGGGGATTTCGATTCCGCTATGTGTGTGAAGCCC
CATCACACGGAGGGCTTCCGGGAGCCTCTAGTGAGAAGAACAAGAAATCCTACCCACAGGTCAAAGTAAGTTTGAAACCTCTGCTCTCA
CTCCCCACGAGCAAAGCCAAAACGTTCAAGTGAATGTGACTGGGATGTTCCTTTGCCCCTCAGCGGGAGTGTTTACATGGTTCTTAGAA
TTTTTAAATAGAAATTCACCTCATGAAAATGACGAATCGTCAAAACACCATTAACATCTCCATTTAAGTGAGATGGTTCCAACATCAAC
TAAGAAAAACTTAACTACCTCTCCAGTGATCTTAAAGCTGTCTTTTCATCTTAAAGTATTTTTAAATATTCTCTGATGTTTTAGTAAAA
CTTGGAGCCAGGTGTCATAGTCCATACCTGTAGTCACTGGACTTAAGAGGCTAGCAACAGCACTGTTAGATATCCCAAGCTAGCCTGAG
CTACATAGCATGCTTGAGCTCAGCCTGGGCTAGGTGAGACCCTTATCTCGTAAGAAAACGAAGAAAATAAAAAATGTCTTTGAGGCAAA
TTTCAAATACTCCCTTTATACAGATTTCATGACAAGACCCTTCACACAATATATCGGCTATGGAGTATATTAATTATTTAAGATCATAA
GTTCAGACCCAAGTGATGGAATAAACAGGTTGTAAGCAGGCTTATCTCACTGACCAAACATATAAATACACAAATACCACTAGCATCTA
CTTCACAACGTGTTTTAGAGTTTCAATGAGGAAATATGCTTAAGTTCTCTGTAGAGACCCTGAGAGAAACCGTGTGCAGCTACCATGAA
TGAGTTCTGTAGCATGGGGGGCAGAGGGGTGGGGGAAGCTTCACTGTCTTTACCTTGGGGTGTCGGGTGTACCTTCGCTGCTCTTCGCT
TTTTACGTTGTTGATCTAGTTGAGACTACCTTCCTCTAGTTCAGAGTTTCTACTTTCCTAATCCAGTGTTGTGTTTCAGAGACTGACAG
TGATTTTCCAAGGAAACAGTTAAAAACTTTCGACTGGTTTAAAACGTTTTATTTATTTGGTAATCTGGGCTTACACTAAATGTTACCAG
CAGTAAACGTTATGGCACTCCAGCCTCTGTCTTTCTGCTGGCTTGAGATCTCAGACGCTCTTGCCCTCAGCTGAGCTGACACTGGGGAA
GTTATCTTCAGTTTTCCCTCCTAGGTTGCTTGTCCCCCTTTTAATGTTGTACTTCCAGGCTCAGGCCTTCCAGGTTCTTGCCCTCTGTC
TAAGACAAAAGAGCCATACCATCCTTTATCTTTCCAGTGAAAAAAATGTGAAAAAGCCATACCATCCTTTATCTTTCCAGTGAAACTCA
GCTCCTGGCTTGGGTCCTGAGAGTCACATTTTCATTTTAAGCCTCCCAGCTCTAGTTGTGGAATGAGCCGTTTTTTTTTTCTCAGTAAA
GGTAACTGAACGTGGAAGTGCAGTGGCTTTAGTCCACAGAACGCCACAGTTATATTAGACAGTGTTTTTCAGAGAAGTCGAACCAGGTC
GGTGAAGACAGGGAGATAGTTAGATGGTTGACTGAGCGATAGCGGAGGAGATGAGAGTAATCGGCCTCAGCTGTGACGATTATGAACCA
CACCAAGACCCACTCTGCAGCGTCTGTAAAATGTGCGCAGGGACACTGCTAGGATTCCAGCACAGAAGCCTGGGCGCCCTGTAACCAAG
GGGACTCTTCACTAAATCTGGGCGTGGCTATGCTTTTCCTCTAAAGGCAGGAGAGGTTGGACTGCTGATGCCCTGGAGGGGCAAGAAAT
GAAGAGTGTCCTCCCTCCCATTCAGGTGGGGCAGTAGCCGGAGGGAGGGAAGGAGAGGATTCTCTGGTGTTTTGGTCCAGCAGGTCTTC
TAGCTGAAGGGAAGGTCTTCCATCAGTCCAGCAAACTTTCCAGTGTCCTCTGGAAACGTGCACACAGCTAGGAACCAAATCCTTTATCA
GCTTTCAGCATCTGCCCAGTGTAGTCGACTGCACACCGGAAACTTCCCTCAGGACAGTCTGTGGCTTCCAGTCCTGCAGAGGCCAGGCA
AGGAATGCAGAGCAGAGCACCGGCCAAGAAAACGGTTATTCCACACCCCTGGTGGTCCTCGTGTCTCCCATCTGTTCCCTCCTGTAGCC
TTATTAACACTGAGGAGCTCAGGCTGAGGTTAACTGGCCCTCTTCAAAGGTGCTGGGCTTCTTTCTCCTGCAGCTCACTCGCCCTCCCA
TGTGAGCGTGTGCAGCTGAGGGGCCAGGGGCCAGGGGCTGAAGAACAAGTCAAGAAGGACTGATAAATCAAGTCAGGGAGGATGAGCAT
TCTCAACAGCAAAGAAGAAATGGGGGCAGAAGTCATCAGGGGTCAGATCTAGAATTTCCTACCTGTGTGTGTCCTGTACAAATATTGTG
TGCAATTATGTGTCCTTATCTGCACCAGGGTCACTGAACATCCCTTGTCAGCACTGATACTGATCATAACTACTTAAGAACCGATGTTA
GGAGAAAAAAGTAGGGTTTTTTTCTCTATTGGCCTTCTACCAAAAAAAAAAGAAAGAAAATGTACATTGTGTTCTATTTATGAACTCTT
TCCTTCTGAGGCACTAGAATTCTGTGCTCTCCCATGCATGAAACATCGCAGTCTCTCTACAAACAGGCTTCCAGGGGTATCATGGCTTA
CTGCATACCCACCCATAATTCATCGTAGAGGTGGGTCCATGTACTTCCTGAGGGAGCGACTCTTCCGTAGCCTTGCACATGCAAGGCTG
TCACTTGCATGAAGTATATGTCTTCAGACTCACCTTTTCTTCACCCCCACTGACAATCTCTGTCTCCCTGGACTCAGGCCCTCTTTCCT
GCCCTGCCTCTTCAAGCACCCCTCCCTGCTGTCCTTACGGAGCCCCCCTGCAAGGTTAGTGGTTCATCTGATGTGAAGATTGCAGTGAA
GATCTGAAGGCTCTTCAAAGCTTTGGGAACTGGTCATGACTTAAGGCTGCAGAAACCAAGCTTTGCTTTATGGAGCTTGTAAACTTACA
TCTTGTTTGCTCGTCTACTTCTTGACTGTTTGAAGCATCAGGTTGACTTGCAAGCTAACTCTAGGAATCTCCCAAAACCAGCACTTCCT
ATTAGAAGTATTTCCACGCTGTGGTAGATGCTGGGATGCAAGCTCAGTCGTCACCCCCTCCCCCCACTGAGGTACCACCTTTTCTAGAA
CTCAGGCGGCTGTACCATGTGTATGTTCCTCTCGTGTAGTTGAGAAGAAAGAACCTCTTTGGCTCTCTAGGATCTTGTTGTACAAGTCT
TCACGTTCTTATTTCCTCTCTCTCTTTTGATTAATTTCTCTGGGATTACTGGTTCCTATGTCCTCACAGGTATCATGAATGGTTTCTTC
AGATTGAAGCATTCCTTCCACTGCCTTCAGGAGCTCGGGGTGGGTGAATAGTCTCTTTTAAACCAGCTACTTCATATTTCAAAATATTT
TAAGCATACAAGTTGTAAAGTTCTATATAACAAATACCATGTACCAAAATTAATAAATTTAATAGATGGTAATATTTTGCCTATTTGCT
ATCTGTCTATCTGTTCTCTCTCTCTCTCTCTCTCTCTCTCTGATTCTCTCTGAAGTACAACATAGTTACAGTGGGAGCCCCTACTCAGC
CACTGCTCTGTCCTCTTATCCTCTTTCTTTGCCCAAAGGAAAACCACTGTTCTGAACATTCTTTATCTGAGATGCATTCATATCATATA
CTAAAATCACTTTCAAGAAAGTTACTTTTTACTATCTTTAATCCAAAGTTTTTTATTATATATGGTGGCTCTTTTTCAAACTATATCCA
GGTTAAAACTGGGCCCCTGAAGGCTTAAACATTGATTGCTTTAATCCAGTCTCTTCCTCTTTATTGTTTGACTAAAAATGTCTTCAGTC
ACAGTGTGGAATTTATGCTGACCCTTAAACCAAAAAATCATATTCATGAGGAAGCTAGGGAAAATAAATATCCAGAATTTAAGAAACTT
TTCCACTTTTAAAAAATGGAGTAATCCACTGTCTAGCCTGTAAGCTCTCCCTCCAGTGTTTGCTTGGTTCTGACTTTATCTGCCCCTCC
TGGGAGAACAGGAGAGACAGCAGAGCTCCTCTGCGCCAAGCCTGTCGCTACCTGTTCTTGACTCAGCAAGGAGTGCCAGGGAAGGCTCA
CTGCCCTCCTTTTCCCGAAAGTGCTCCACAAAAATTTTAAATCAAACTGTCTTCCTTGAGCAAATGGAACAGCGCCAAGAGGAGAAGCC
ACACGTGGCTTTAGGAATGCGCCTGCAGCCCGTCCTCACTTGCTTGAGTCCTTCTGTGCTGGGCACCTTGTGAACATTGCACTTGGACA
TCAGTGACTTTCATTTCTAAAAGACAGTTTGTTTGTAATCATCCAATGCCAGGAGGTTGGAACACAGTGATGCTAATATGACGATTCTG
ATCAGCTTTGCTGGCAGCTGCCCTGCCTGCCACTCTCCTCCGGTTTGATCCTTAAACAACCGGTTTATTGGAATGTTTCTGTGAACAAC
AGCTTTGGCTCCGTTCTCCCACCTCCTGTAGTGTGCACTCTTACGTGCCTCTCACTGAACAGTCCTTAATGTTACGTTTCCGTCAAGGT
TAATAAAGCCCGGCTGATCTGCTGTACGCTTGAGGATATCACATATCTTTGCAAAAGCGCCCTTTCCCTTGCACAGGCTTGTCTGCATC
CGGAGACTTGCCATCCTAAAGATCTACTTGCAATACTAATTTTGAATAATTTAGCCTGATCCAGTTACCTAACATGCCAGGGGGGAAAG
ATCACCACAACAGATTGGGCATTCAGAACCTACCTAGTTCATAGCATTTATTAGAATGTCCTTTGACACTTCCAAGTGTTTACTGTGCT
CAAATAAAAGAAAAGAGCTATCTTCCCTACCAATACTATAGTCTGTGCTTTGTAACACACACACACACACACACACACACAAACACAGA
GTTTATTAAAACAAGGGCTTTGTAACCCAAGGTTCATGGATCCTTTTGTGGTTTCAGAAAGGTTTGCAACCTCTGACTCTACACATAGA
GTTTTATGCATATTTGTATGCATTTTTAAAGACTCCAACATGTGTAAAAGTAATGCTGAAGGGCATTGGCTTTTTTACCACAGCTGATT
AAATCCATCTCTACAATTTTGGAGGGATGGGGGCGGGGGCGGCGCGAGGCCGACTGGCTCTCACTGTGTAGCTTACCCTGGCCTTGAAC
TCATCTTCCTACCTCTGCCTCCAGAGTATTGACAAATTCCAGGGATGCAACTGTCTTGAATGTTTTTCTTCTTTCTTTTTTCATTTCAA
AAATTTACATTTACACTTTTTAGTTTTACCCATTTTAGGCAAGTTGAACCGCTTTGTGTTTCTCAGCATGTAATTCAAGCTAGTTGCTC
TTTAGTATTCCATTGACTGGAAAAGATGTGAGATTTAGCGCCTCCTCACACACACACACACACCACACCCCACCCCCGCCTTTCTGTTC
TCTCCTTCCTCCCCTCTCCTCCCTCTCTCCCACTCCCACCGACTCTCTTGCCACTGACTAAATACGATGGTTCAAAACCAAAAAGAAAA
CAGAATCATTTCCCTGATATTCACTCACCACGGTGGCAAGTCCTGCACTACAAGGAGGTAACATCCTCTCCGTGCCCATAATAATCTAG
ACTTCTGCATAACTTTTCTTCTACGTATCCCAGGCGTTTTGCGATTGCCTCCATGAGTAAAATACTGACTTGTTAAAAAACAAACAAAC
AAACAAAAAAACCACCCCTTTATTTCACTGTTAGCATTAGAACTTAGAGAAAGCTGCTTACTGAAGGAAACTGTTGAGCAGTGAGAATT
GATCCTGAGAGTTAGCAGAGCCAAGCTCCTGAGGGTTGCAGGTTGTCTGACTTTCCAGGCTTGGTATGGTGGCCTGCCTCGCCTAACAT
TTCCTCTTCCCCTCTCCTGTTTCTCTGCATACTCATTGCTCTTGGGATGCTTGTTCCTAGTGCCAGTGTTCAGTCTGAAATCATCTCTG
ATCAAAGTGGTCTCTGTTTGAAGAGGATCATTACATTCAGGCCGCCAATCCCGGTTTCGTAAAGCCACATCTGAAGATTGGTTTTGATG
AGGCAGAGTCAGTAACCAGAGCCTATCATCCTCATGTCCCCAGCCACCTTCTCCATAGTCAGCGTCTAGGGTCTTTGGTGGGTCTAGGG
ATCTTCTCCTCGCTCTCCTGCAGTCCCCATCTTCCCCCTCCCACTCTAAGTCGATTTTAGGTCAGTCTTCCAAACAACTTTCACTTTCT
TGAGGTCTAATGGAGGTGAGTGGGAGGATATCTGGGCAGACATGTGAAACAATCCAGTCTGAAGACCAGGGGAATGCAGAGTAAGTCCC
CAGCATCTTCAGATAACCGAGTTATCTATCCATTGCTTCATAGATAAGTACAAAACAGGTTTGCTTGTATTAGAGAAGACTTGGAATTG
AAAACAAGCCACAAGAAGCTAAGGCCAAACACACATTTTTCAAATCACATATCATTGAAATGGTCTAGACATTCGAAAAACAATCATCA
GGTCGCACAGTCGATAATGAGGTTTCTCGGGGTGGAACAGAGAACCGTGGAGGCTGGGAGAAAGGGAGAGCTGAAAGCAGAGCACAGGG
TGTCTAGCTGAAGTCCTGGGAGAAACTAACATGGCTGCATGTGTGTCTCTCTCCTTCTCTACGCACAGCTCTCAGATTGGCATGCAGTA
CTCAGCTTGTGGGCAACATATTCACGGTGTTAAGCACCAGCTGTGAACTGATGGCCAGCGTGGGGTGTGACATTACTTCCTAGATTCTC
ATTGGCTAAGCTTTGTCAACTGATGGCCAGCGTGGGGTGTGACGTCACTTCCTAGATTCTCATTGGCTAACCTTTGTGAACTGATGGCC
AGTGTGGAGAGTCTCACTGGCTAAGCTGCAGCTTCTTCATCTGTAAAGCTGACGTAGAGAACCAACTCAATGCAACATGTGCCTACATT
CAGTTGCTCAATGCCGGCACACAGTCAGCCTGGAAACACAATTACACCCGTTTTCATATAAAATTTCAGGAAATTGGGTACTACTCCAA
AAGCTTGTGTTTAGAGGTAACACATGATGTCATACGAAACTCACACAAAACAACAGCATAGAAACAAGTGTTAGGTTTTGTTAGGTCCC
TTCACTGTGTCCTGGTAGGCCCTATGTGTCTAATTTGCACAACGCTGTATTCATTCCTAGGTTGCCATAACAAATAACTGCAACCTGGG
TCATATTAAACAACACAGTTTATTGTTCCAAAGTTCTGGAGGCCCAGAGTCCACAGTCAAGTTGCCAGTGTGGTCTTGCTTCCCACAGA
GCCTTCAGGAAAGAGTCAATCCTGGCCTCTTCCAGTTTCGGGTACTTGTTGGCAGTCCTCAGCAAGCTCTAGCTGCAGCCACTTCTCTG
ATCTCTGCTTCTGTCTTCACAGGGCTCTTCTCCCTGACTTTGTATCTGAATCTCTCCCTCCTTTCCCTCGTAAAGACACCAGTCAGCGT
GGTTAGGGACCACCACGCTGTGTAGCAGGACCTCTTCTTGACCAATCGCATGTGTAGGAACCCTGTCTGTACATGAAATCATGTTCTTA
GCTCCTGCATAGGCCCGAGTCCTGGGAGATGCTCTTCTCACTTACTACAGGCACGATGTGCAGTGGTTACAGAGTGTTCCCGAGTGTGT
GCATTTAAGGCCATGTCGTTCCGTGCTCTGCTCAGAAAGAAGCCTGTGTGCACCACGTGCTCCCCTGTGTACCTTTGTTACATTCAGGT
AGTGCAAGAAAATCTCTCAGGTCTGAGGAAATTGCTGTACTAGGGTTTTGAACATTTATTCTAGTAGATGGAAGGTGTGCTAGAAAGTG
TCTGCCATCAGACTCTTGGATGGCACTCTGAATTGTAGCATCTGCCAGTTTCCTTCCCATAGTAAATTTCAGGCTGCTAACATGAAGTC
AACTGGCTTGCAATAAATAAATAAATAGATAAATAAATAAATATTTTAAAAAACAGTGAAAATTTAACTATTCTGAAATTAACTCCATG
AACTAGTCAACCGGTTTCTAACCTACTAGGTGTTAAGCCAGCAAAGTTAGAGCGAGAGAAGGTGATACATTGTTTCCTGTAGGAGCTAC
TTTCTATGCACATGTTACATAATATAAAGATCTGCAGGCTATGACGGAACCCCATAGGTGTTACAGCTGGGACTTCATTGTGTGATAGT
TAAAGAGAGCTGCGGTTGAGTGGGATTACTGCTGTTTAGAAGCAAGGCTAGACCTCGGTCCCATTCTGCCACTCGCTCGCAGGTGGGGT
GTTCTTCAGAAAGTTGCTAAAGATCTTCAAACTTGGTTTTGTTTATGAGATGAAAGTAGAAACAACTCCTTCACAGGAACCATGCGTGC
TTACGTAAAAAACTGACCTCTTAGAAAGGCAGCTGCCATACTCCTGGCACATCTGCCTCATTAAGTGCTCATTCGGACACGTGTGTAGA
AAGAGCCATGGAGATGACTGTTTTTTTCCTGAGAGCAGAAGCTTGCAGGTACTTTGTCAACAGCACCTAGAAGGATGCCCATGTATAGC
ACACACGCAGCAGTTACTTGTACAGTGAGCAGGTGGTCAGTGCTCAGAAGCTGGAAGGATCATCTAAGGAGGCACTTGTCCTCCCATGG
TGGCGCTAAGCAAGTCACTGGAGTGGCCACCAGGTCTCCACCTGTCACTGCTGCTCACTCCCCACCAGCCTGCTCCTCTTTCCTTCTCC
AGCCTGGCATGACTGCCAGCCAGGACAGCCACATCCCAGACCTGGCTGTACACTGTAGTGTAGCTCTTAGACATAGCACCAATCTATTC
TGGGTTGAAACTGGCTTTTCTGCCTTACTTTCTAAACCTATTTTAACTGTCCAATTGCAAAATAACACTTAAAGAAACTAAAAAGGTTT
AAGCAAAATAGACATTTATTTATTTTAAAAACTGAAAATTCTAAGGGAAGAAGTAGAAAAAAGAAACCCAAACAAACAAACAAATTTGC
CAAATCCGTAGAAAAACTCCCATTGGGCTTTTTAGCATCTCGACTTCTACCTTAGAGGCCACTCAGCAGCAGCAGGCTCACTGTGAGAA
GACCTGCATTCCCAAAGCTGACATCAAAGCTTATGAGACACCCAACTGGCAGTTCCTAGGCCTTTGGTACTCTGAGTTGTTCCACTGCC
TGCCCCTGCAGCAGTGAGACAGGGGATAGGGTTGAGCCCTGTGTCCGTCAGTCGCATACTCTGCCGCTGGTTCTACAAGTGGGATTAGT
CACTGACTAGCAACATGGGGAATGAACTGAGCTTAGGATTCACTGAGCAAAGAGCTATGCGCTAGTGACACGCGGATGCTCCAAGATCA
CAAGGATTAGCTCATTCGTCCATCCCGTGACCAGCCTACAGTTTCTGAACTCTCATTCCTAACAGTTGGTAAACCCGGGTTCCTCTCTT
CAGAATGACTGTTCCATGCTACTCCTAATATATGAAACTTTTGTTTTTAAAATAAACCAAAAAGTTTTTTGTTTATTTACAGATATCAC
TTGCATTTAATAAAATTTCAAGAAACACAAACTTATAGCTAGTAAGTCCCCTTCTCTCCAAGATTCCTTTGTTCCCTGGAGAGGAGAGC
TGTTAGGGGCTTCTCAAGTGCACTCCCAACCGTGCCTGCCGTGTGTTTACCATGTGTGTTTATACCTGCTGGCACACTTCCACTCACGC
TTCCTCCTGTAAGTAAGCATTAGTGTACCGCTTGTGAGCACCGATGTAGTCTACTAATCTATCAACCATCTGCTGCTTACTTTTTTCAC
TTACTGAGTCTGAAATTTGCTCAGATTAACAGCAAACTCCAGAGCTGGCAAATCCAGATTCAAGTCCAGCTCTGTCCCCTGACTAACTG
TGTAACCTTGAGCTAACTGACCACCTTCTCTGTGCCCCTATCCAACTAGCATAAGAACAACTCCATGTCTCATAGTGTTGTTGGGAGTA
CTAATTGAATTAGTGTTGAAAGAGCAATGTCTGCTCCAGGGAACAGTTCTGATGTTCAATTTCCTACCCATCTACTCTGTAAAGGCTGT
GTGCTTTCCATGGCTGCTTGGGGCATGCTGTCTTCTCTAAGCTCCATCTGTCTTCTATCACAGGCCAACCTTAGTAACCCTGCATGATA
GTAACTTCTCCCTTATCCTCGGGTCTTTGTCACTTTGGGATGTTTTGTTATTGAGATAGCTTTTTAAAAATACTTATTCATTTTTATTT
TATGTGTCTGAGTGTTTTTGCATACATGTATGTATCTGCACCATATGTATGCAGTCCCCACGCGGGCCACAAGACGGCGTTGGAATCCC
TGAAACTGGCGTTGCAGTTGGTTTTGAGCAGCCATGGGGATATGCTAGAAGAGCAGAAAGTGTTTTTAACCACAGAGCCATTTCTCCAG
CCCAGAGATACCATTTTAAAATTATAATCTGTGACTGTTTTGTTTTGTTTTGTTTTGTTCAAACTAGGTTTTACCATCAAGCCCCCTAG
CTGGCCAGGAACCCACTGTATAGACAAGTCTGGAATGGGACTCACAGAGGTTGGCCTGCCTCTGCCTCCCAAGTGCTGAGATTAAATGC
ATGTGCCACTGTGTCTAACCCTTTCTTAATGTAGTGTGTTTATTCTTTGAAGAGTCATAAACTAGATTTTCACCATATTCACTTCCCAC
TGTTCTTTTTAGCTCTTCTGAGGTCTACCTCCTACCTCCTCTCAGTTTCTTCCTTCCTTCCTCCCTCCCTTCCTTCCTTCCTCCCTTCC
TTCCTTCCTTCCTTAATTCATTCATTCCTTTCTTTCTGCCTATGTACTAATGGATATGAGACTATCCACTGACCATCGTCATTCCTATA
AACATTTTCCTTTCATAAATGTTACTCTATGCATGTGGAGGTCAGACGCAGCTTTCTAAGGTCAGTTCTCACTTTCTGCCTCTCTCTCT
TGTTCCCCACTGTGATCCATCCTCCAGACTTGATGGCCCTGGAGCTCCCAGCTGGTTCTCCTGTCTCTGCCTCCCATCTCTCTATGGGG
TGTTGGGATTATAGATGTGCATCACCTAATAGGCCTTTTTACCTGGGTCCCAGGGTCTCAAACTCAAGTTGCTGTGATACGCATATATA
ACAATGCTTTTACTCATGACCCTCCCTGGATGCTGAATTTTTTTTTATCATAGCTTTATTTTCACTCTTTATTGTGCCATTTGCTTCTT
ATATGTTTAATGATGTGTTTTTCAATGTGATGACCCTTTCAGAGAGTCTGCCCTCAGTTGTTCCCAGGCCTCCCCCACTTCAGTGTCTT
CTGGGGATGCCTTCCTAATGAGCCCCATGCGGACTCTGATGGCTGCCGTACTATGTCTTACTCAGATGTGCCCCTCCTCATTTCTAGAC
CTTATTTCATGTGCATAACAACTGTGTCTTTTTACATTCCCTAACACTGTGCTCAGTTAAATTCCTTTTCATTCTTGGGTACTAGGTGA
TTACCCCAGTTAGCAATAGAAATATGCAATTAGTATTACACATAAGAGGAAACATCTCTCTCCCGTTCATCTCTTACTGAGCTCTTGCT
GTGTCTCACAGCCAATCATATTCAGCTCCACCAGGGAGGCGTTGGTATCCTAGTTTTCAATTGGGTTTGATGAACACTTGGCCAAAAAC
AGCTTGTCTGTCTACCTTTCTTATGTACATGCACACACACATATCTCCTAATTACTTTCCCCAGCCTAGACTTCTCCAGAAAACCACCC
TTCACATCTGCCTACTTCCTCCACTTTACCATTTGGATACCTCGTAGCCTTGCTGAGCTCAAAGTTGAATGCTTGATTCCCTACTTGAA
GTTGTCCACTCTTAGAAAATGGTAACCCATCCCCCAGTTTCTCAGACCAAGCGGTCTTCAAGTTATTCTTCACGCTCCTCTTCTTGATC
ACCACTTATAATCTACCCTACTGGCTATTTCTTCAAACTAATTAGAATCTACTTGCTTGTTGCCACCTCCACCCCATCACCCCTGGTAT
AAACCATCCCCCTCCTAATCCCTCCCATTTGCATCGGTGATGCCTGTCTCAAGCAGGTATTGCTAACTGATTACAAATTTAGGGTTTGC
TCTATCATTTTTTTTGTTAGATAGCATTTATCTTTTTGTACTTTTCAAAGTAGTTTTCTCTTCTTCACACTCACTGGGGGGTATTTCAG
ACTAGTTTAAATGTTTCTAATCTAAAAACACAAAGTCAAAAAATGCTCTGAAAGAACCAGGGGAAAATGAATGGGGGTGGGGGGGGGGG
TAAAGTACTTGTCATACAAGCATGAAGAGCTTGAGTCCCCAGAACCCACACCACAGCTCAATGCAGTATAGTAGTCCAACCCTCTATAA
TCTCATTGCTCCTGTACAGAAATGGGACGTAGGCACTGCAGACTCCCTGGAAATTTACCTGTGGTCCTGAGCGTGTCAGCTGACTCCAC
TTCTGGATCCTCTTCTCATCCCCGAGCCACGCATCACTCCTGCCATGATTTACTGTGATATTTGAACGCTGCTTGTCACCAACCAGCAG
AAGTGTTGCATGCCAGCTCTCGTGTCCGCTACACCTCGCTCCCCCACTCCTAACTGACTGGGCTCATCCTCCATCTCCTCCTCTGTCCT
GAAATCCCTCATCTGTCCAAGGGACCTGATTCCTTAGCAGAGTCGCTTTTCCTACGTTTTCCTGAGGCTCCATCATTGATCTTTTTTGT
TGTTGTTGTTTTTAAGTCTTGAGTTCAGAGCCTATTTCACACATCTCATTGTTGCCATCATCACAGACTCTCCTTCCCTCCCCATCCTA
GCTTTGTATCTCCTTTTCCCAATAAAATGCAAACCCTCATTCCCAATAACACTAGAAGTGCCTACTCAGTTGATTGGTCCTACAATACT
TCCAAACACATGGGGAGATAGTATGGTTTCAGGATGACGACGCCAGTATCAGCCCTACCAACTTAACAAAGAGTTCCCTTTTCTTTTTA
TTTCTTTAACCAAAATTCAGCAGCCAATAATCAACCAGAATACCGTTTTCATGGGTTACATTCTTTTCCTTCTAGGAAACTCCCTTACC
ATTGAACTATAGGCTTAGAATACTTTGTTTATGATGAATCTTAAGAGTTTTCCTATTCCTGCTGATTTTGTTTTATTTTTGAATGGTGG
CATGTATCGCCCTCAATTACCTTCCATTTGTGTCAGATAAACTGGACGATTTGGGAACTATCATAAAGTCCGTTTGCTTTATTACCTTT
TAATCACAGAGAGGAACAAAGAGTTCTGGGATGGTTATTCTCGAATGTGAACCCAGTAAGCTCACGTTGTCACATGGGGTGTTCCATTT
CTATATTTGGAGCCCTCTTAGTCTTCCTCACAGTCACAGAGTACACACATATATGGTTCTCGCACATAGGCTTGGTGCTGGCTCTGCGG
TCACAGACACGAATGAGTTTATTCCTCTGCCCCCAGGTTACAGAGAGTAGCCAAGCAGCAGACACGTATGAGATGATACTGGGGTGTTT
CCTTGGAATTTGGGGAGGATGCTCTGCAGTGGGGTGCATATGTGTATGTGTGTGTATGTTCCTGAAGTGGGTGCCCTTCCCTGAATGCA
TTCCTACTGTGTAGTCATTCCAAACTTCTGATGGTGGAATGCCAGGATCACAGAGCACTTTTCATACTGGATGTCAGAGGCATGGCCCC
TTCACAACACACGTGCTTCACTGTCAGAGCGGTGGGCCTGTCACACTGCGTCTGCCTCAGAGATAACATAATCTTCCAAGCTGGTGATG
TCATCCTGGAGACACACAGCCAACGATGAGCCCCAGGGGATTGTTCCTAACCATTTTAACAAGTCTCACCCCTAAAGTCTTTATGGTTC
TTATCAAGTTTTTTGACAAGAAGTGGTAGATAACAAATACTTTAAATTCAAATTAATCTTGAAAACTGGAGCGAAGTGCCAAACTCTTC
AGCCTTGATGTTGAACAGAGACACACACATCCAGAAAAAAAACAATGAATCCACAAAAGTACACAGCAGAGGGCTGAGTCACCAGCAGT
ACATGCGGAGAGAGTAGAGTTGACGTGCCTAGCAAGAGCTAGCAAGGCCTGCAGCTGCCAAAGAAGTCGCTCCTCACTAGGCAACAGTA
ATGGAAATATGATGTATAAGAGCCAAGAGGTGATAGTCTCATGGCCACCCTCCAGGAAGATCACACTAGATGCGTGCAGCCTGCTGGGC
AGAGCACAGTAGTCGGCTGTGCCCGGGGCGGGGCCAGATCAGGATGGTTGTGTGACCACACTGGAATGGCTTACAGCATGCTCCTGGCT
GTCCTCGGTGTTTGAATAGCTATTATAAGAAAATGGAAATCGACCCCACCATCACAACACAGGGGAAAGAGGGAGCGGTGAGCTGAAGT
CCCACAAGACACCACCTTCGTGGGAATCACAGCCACCACAGGACGGGGTAACTTTGTCGTCACTGCTCTGAACTAACCTGAAAGACGGT
AGAGGAAGAAAGGGAGGCTGCGTGGCCTTCAGCTTCACAGCCTCGGAGTCTCTGGCTCTCCATCCCTGTTTTATATGGCATTACACACA
GTTTTGCTTAGTAAATCATACCTTTGTAACAAGGGCTGATCAAGATTTGCGTACAAGGAAGTTATAATTTCATAATTTCATGATAGATA
AGAAAATATAACAATAGATGCTAGTGACTTGCTGGCCATTTTCTCCATGGGTCAAAGTGCATGACTTATTTTTTTACACTAGTATGTCA
GTTTATTAACACAGGCCGAACAGTTTAGTCACACCTTGAAAGGGTTTGGTTTGGTTTGGTCCTGAAAGACCAGGTACAGAGTGATACCA
GATTTTTCCAACAACTTGCTGGCATTATCCTAGAAATCAGTAGAACTTTTGGTGTTTACCCCTCCATGCCTTGGTCTACATCATGCTAT
GCAGATGGCACACTGCTTTCGTTAACCTCAGTCTGACACGCAGATCGTGAGTTCTCTTCTGCCATGCTCCTGTCTGGCGGCGTGATGTG
ACCGATGAAGTGTCACCAAGAAATAAGACAAAATTTCCCAGGTCTACTCACTGGCAATTCTCTTTATTTAGTAGGGGTTTTCCACAGGA
AATTGCAGGAGAAGAGTTGAAGTGCTCTTTGTCTTGTGTACTTTCCTCTGACTTTTCTCCTTCCCAGAGTGGCCCACATTGCCTTGTAA
AGGAGCTGTGTTGAGCATGATACTGATCAAAGTCAGAGTGCGTTCTGCCAGTCAGCTAAAGCCTCATTGCTCCTCTGCAGTGGACTTCT
GTAGTTAAACTCTGTTTTCCAAACATGAAAACACATAAACAAGCACTGGAGATATGGCTCAGCAGCTAAGATCACATACTTCTTCTGCA
GAAGACCCACGTTCCGTTCTAAGCACCCACATCGGGCAACTCACAACTGCCTATAACTCCAGCTCCAGGGGACCCAACACACTCCTCTG
GCCTCTATGTGCACCTGCACTCATGGGCACATATTCACACATATATACATAATTTAAAATAAAAATCTTTTAAAAATTAAGGAACATAT
ACCCACAGAGTAAGTATTTGTCTCTAAGAAGAAATAAATCGGTGATATTAATCTGAAACAAATTTGGACATCACAGAATCAAAAATATT
TCTGATTATCCTAAATTAACATCAAAATCATTTTAACCGAATTCTGGAGAGGTGGCTCATTGGCTAAGCTCTTCTCTCCTCTTGCAAAA
GACCCAGGTTTAGCACCCAGGACCCACATGGCAGCTTCCCAACTCACACCCATCTGTTGTCCCAGTGCCAGGAGACCTCACACCCTCTT
CTGGCCTCTGCAAGCACCTGCACACACATGGTGCACGTATTTCAGGGAAAACAATTATAGACATAAAATTCAAATTAATTTTAAAATGT
CATTGTAAGATCATGCTAGATAAATTTTCTATAAATCATGTCTGTTTTCAGACAGGGATTCTGTACAAATCTAACATGCATTTTAGACG
ACTTAGTTTTGAAGTTTACACAAACTTTTTTTAAAACTTTGCTTAAAATAGCTAATTATGACTTCATTTGCCTGTTTTGGCCCTGCCCC
CCATGGCCAAAGCCTGCTGACATCCCCAGGACCAATAGTCCCTTATTCTCAGTACTATAATAAGTACTGAGCTCCCAGCGCACAGCCTG
CACTGCAGTCACTGAAAAGCACTGTGTTGAGTACCCAGACCCAGCCTGGATGGGAAAAGTTAATATCATTGCCTAACATGAAGGTTTTC
TTTGTCTCTAAATTTTGATTTTTCCATGCTATCAGAGGCCCCCTTGCAACAGGTAATCTGAGTGTTGTCTGTTTTCTCCGTCCGTCATC
GTGGTTAAGTGTATTTTAGATGCTCACAATTCTAAAGCTAAATTGACTTGACAAGGTTCTTTCCGACCCAAATTCTGTGATGTCCGACT
CTTTTTTTCTTCTTTTATTTTGGATTTGTGATTGTTGACCATTGTCCTGTATGTCTTACATATATACTTTAGGGAACCATAAGCATTTT
AGAGCTGGAAGCCATGCTAGCTGTGGGCTAGGACTAGACATTTTGCAAAGAAAGCATTAGGAGACCAAAGTGGTTAGTGATTCAAACAA
ATTCATAGCAGTAGCTAAAGACTAAAATTCTGACCATCTTCAGAGGACTAAAAATAATCAAAGTGTTATCTGAAAATTTGTAACTCCAA
GCCAGGTATATATATATATATATATATATATATATATATATATATGTTATGTATATATATTTTACCCCAAACAAATTAAAGCTTCCTAT
AATTCTGTACTCATATATGTATATGGGGTGTGTGTGTGTGGAGAGAGAGAGAGCAGGGAAAGAGAAGGGGAATTAAATATTTTTGATAA
CGTAAGCTTTGATAAGATCTGCCTGACACAAAATACAAAATGCATTTGGTATAAACAGCATCATTCTCCCTTTATACCAAAAACGGAAA
TTTCTTCGCATTCTATACATTTGAGAATGACACGTCGGGNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNUNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNCCGTGTTTGCTGCTCTGTACTGGCTATAACCTGCACAAGTAATGGCCTC
AATACAACTCTTTTCTAATAAAAGCATGCCTGATGTTATACCTCTAAAGTAAGATTCGTTTATCTAGGTCTGCCATTGAGAGTGGTTAT
GGGAGGATTCCTGTCCAGCATTGGGACTGTATCTAAAAACAGCAGTCTAGCTAGAAAAAAACAGCACCTGTACAAAAAAACAAAAACAA
AAACAAAAAAACCAGCAGACTCAAAATAGGAATCTCACAGGTCTAATCGGAATCCTCACTACTTCTCCTGCAGAAAACTATCCTTAAGA
GACCCCAGGTCAAATAGTGTTCCAAAGAAAAACAGCCACATGTTGTGCAAAAAAGATAGACTATTGCTTTGTTTGTAAGGAGATGCGTC
AGCCGTACGCGTTTTAATAGAGTGGTAATGAGCATCACTAATCCCTGAGCTTCAGACCACAGACTCGGCGTCTGTCTGCAAAGGGCCCT
GAGTTGGAGCACTCTTGTGATTCATTCCTCTTCAGTTACACTTACACTTCAACCTAACATGGATAGGCAAGGCACTGAGATCAGAGGAA
ACGCCTGAQCACCGGGGGAGGGGCAGGGGACGACGACCCAAGTCCCCTGGCTCTCAAATTTGGCAGTTAAGTCCACATAAGAGGCAGCG
CACTCCCACACACTACCACGCTTTTGGTACAGTCGCTCTGTTCAACAGGAGGCAACGTTAGCTTTAAGAAAAAAGGTAAGCTCGGCGTG
GTGGTCCACGCCTTTAATCCTACCACTCGCGAAGCAGACCCAGGCCCATTTCTGAGTTCAAGACCAGCCTGGTCTTCAGACTGAGTTCC
AGGACAGCCAGGGCTACACAGAGAAACCCTGTCTCGAAAAACCAAAAAAAAACAAAACAAAAAAAAAGAAGCAAGGTAATTAGTTAGGC
TTCCGCACATCCACTGATAAACTGCTGTCAACAGAAGGCCTTCCTGCTAAGGGCTAGAAATCTCACACATGCTTATCTGAGAGATGAAA
ACACTGGGTTATTGGAGCGGGCTGTGAGAGCGACCGACCACACCCCGCCCATCTCTTTGACCACAGGACCCTAAGCAGACCCAGACGTA
TCAGGTCACAGTGGTGCTGGGGCTGCAGGCGGTTGTGAGCCTCTCCAAACAACCCCCCCATGAACAACCAAATTCACATCCACTCCAAG
AGCAGCACCCTCTTAACCAACAAGCATCTGTCCAGCCTGTACACTCTAACTTAGTATCATATCGAGTAGATGACACAACTCCATCTTGT
GGATGGATATCAGATAATGTGATGACTTCCAGAAAGAGACTAAGAGTCGTGGACCGGCGCTTGTGAGCTCTTTCAGAAGGGGAATGTAG
CCAACCAGCTGGTGATGGCCGAGAAGCGACCTCACACCGACCACGTTTCACGGATTGTCATAGGTCAAACCATAAGTGACTCATGGGTT
TCCAAACTTAACGCAGTCACAACCGTTAACCAACCTTTATTTCAAGACTAGTAGAAGTACTTAACTTTCAGAACACTTCCATCTGAGAA
GCTGTTTCTCCCTGGTGACTAGAGTTGTTCTTGAACGCTTGGGTGTAGAGAAAGGAATTCAAGCAAGAGTGGGTAGACTCTTTTAAGAA
CCTTTCTCACAGACTTAGAAGAAGAAAAAAAAAAAAAAAACGTCAAATGAAACAAACAACCCCCCACCCCAAGCCAGCCCTGTTTTCTT
GGAGTGGCACCTGCCGCCATGTGTTTGAATAAATGATAGCCTTTACCATCATCAACAAAGGGCTGCTATTTTTAGAATAAGAAAGCAAA
GGAACTATGTGGATAGCTAAGGGCAAGGGTCATTTAGATGCTTTCTAGCGGTCGACAGTAATTAGGCGATCTAGAATAAATATTGTGGG
TAAAACAAGATGGCTTTTCCTQGAAAAGGGAAGAGACACAGTTCTCTGAATTAGTGACAAGAACAGGTCCTGAGCTTCAGTAACAGAAA
GGAGACACATTTGTCCAGGACCATTGTGGAGGCTTCTCGTTAGATCTCAGGAAATCCCAGCAGCAGATGTCTGTGAGGGCAAGAAGAGA
TAAAGGAGAATTATTTTGCATCAAGAACTGTTCTAAGCAATTTAAATTATCTCTTCTTAATCCTTGTGAAAACTCACAGACTATGACAT
TGAAAGTTAAACAACTTTCCCAGAGCCTGATATCAGCTTGTTATGGAGGGTCTAGGATTCAGAGCTATTCTGTCTGACTCCAACGCCTG
TGTTCTTATGGTGAGAGGACTCACTAGTTTCAGGTCTTGGAAGTTCAGCCCTATATCATAGCAGGATGAGAACTTTGAGGGAGGTCCAT
CTGTCCATCAGAGAAGGATGCTCTAATATATTGGTTCTCAACCTACCTAATGCTGTGACTCTTTAATACAGTTCCTCATGTTGTGGTGA
CCCCCAACTATAAACTTATCTTTGTTGCTACTTCATTGCTGTAATTCTGCTACTGTTATGAATCATAATGTAAATATCTGATAGCAGGA
TGATCTTGGGTAACCCCTGTAAAAGGGTGGTTCAGCCCCCCAAAGAGTCTCTACCACCGGTTCAAAACTCCTGCTCTCCTCAGAAATTC
TGAGGCTGAGAAATTGGGGTGGAACAAAACTGTAGACCCTGTAAGTCTGACAGAAGTTAAGATAAATGGACAGAAGACAATGTGAGGCA
GATGCACGACACAAGACGCCTCAGGTCACCACCCAACTGGAAACAGGAGGGAAGGCAAAGGAGGGTTCTGACTTCATGGGGACAGCTGT
TGGCTTGACTTGACTTTCTGGTGGTGATCACTGAAGAGTCTATTTCCACAGCTGCTGTGATTTCTTTGGAGGTTTCGGTACCAATTCTG
GTCTACAGACAAGACCAGGGTCCTAGACACACAGTTAAGCTTAGGTTGTTCTCTGGTATTTAATAAACAAGCTGTAACTCAAATAGCTT
GTTGAGAGGCTGTGTTGGGATCCACCTATCTTTAAACGCAAAACAAAAACCATATATCACTTTAATTTCTTCCTACCTAATATAACTGT
TTACACTTTAGTAGTTTGCCTTCGATAAAGAACATTTAGTGCTATCTCAATTGTTAGTCACGCCCACGTTAACAAGGTAAAGTTACAGG
AAGTACTGTGAGCACAAGGAATAGGAAGGACGGGTGAAAGCAACGCTTCTGTTCTTATAGTCCTTCCATTCTCACATAGAAGATAGGAC
ATTATTGATACAGGGGACATGTTACATACTTAACAGTTATTAACATACAGAGAAAAGATGAAGCAGGGCAGAGAGCAGAAGATGGGGTG
GTCGGAGCTTAGGTGGAGGAAGATTCGAAAAACACTTGTCTGAGAAGAGAGCTGTGCACAGACTGGAGCTGGACAGAAGACAGCAGACA
TCCTCGAACAATGTCCCACCTAAGCACGCAAGTCCAAAAGCTCTCTGGTGGGAGCCCTCTTGGCAAGTTCTGGAACAGCAGGGTCTACT
ATAGATGCATTGGAAATACAACAATCAAGGTTGAATCAGGACCCCACTCCCCCAAAACCCCCGAGGCTCCCTCACCCGCTGTCGACGCT
TTTTAAGAATAGAGACATTAAAATGTTTTATTTACTTCTGTTCTTTAACATGGGAAATTGTAACCATGTATTCCCAGATCTGTGTGTTG
ACGCCCTTGTGCTAATCACTCAATTAACATGGGAAATTGTCATGTATTCACACAACTGTGTGTTGAGCCCCTGTGCTAATCACCCAGTT
CCAAGTGTTTTCATTTTTGGAATTTTGAACTGAGGAATGATGACTTACCATGTCCTGACTTGAACAAAACTCACTCAGAACAGGTTCTG
AGGAGGCACGCAGGGAGCCAGGGACTAGCCTGAGAAGTGGTTATTTGGGGCTTCGTTTGAAGATCTGGCTTGATTTTATTTATTTTCTA
ATTTTTTTCAAAAGAAGAACTGAAAAGGCAAATAGAACAGTGAAAAGAGAAAGCTAATAAACACGACCCAGCCCAGGCAGCACCCCTTC
ACACTTCACCTTCTACCTAGACGTGTACCTGCCTGGCAAAGGGCTGGCAGAGGGAATATTTCTAACACCATCCTATAAATGACAAGCCA
CATTCCTATGCTCTTATGCAATCATGGACTATTAAGCCCCCACACAAAGGTGAGGATTTGTCACATGTCACCAAAGCATTGAGACCCAA
TTGTACCTGAGGTACCTAGGGGCGCTTGACCTCTAGATATTTATGATGCATGTGATAAGTTTGAAAATTAAAAGGAAACCAAAAAGAAA
ACAAGGAAGCCCTGCATCAAGGCTGATGAGGTGAAACTTTCAGAACACGCGTACATGCAGGTAGACGTGTTTCTCTCTTCTCTTCTCTT
CTCTTCTCTTCTCTTCTCTTCTCTTCTCTTCTCTTCTTTTTTCTCTTCTCTCTTCTCTTCTCTTCTCTTCTCTTCTCTTCCTGTGTGTG
TGTGTGTGTGTGTGTGTGTGTGTGTGTGTGTGTGTGTGTATAGACTAGAGTTTATTCAGGGCCTGGGGAGGGGAGTTAAGAGGGTAGTG
GAGTCACAGAAACCCAGACAGAGCGACAGAGTACAGAAGTAGTTCCTGGCCATGACCATGTCGAGAGAGAGGAGAACGGAATGGGGACA
GAAGGGGAGCAAGAGGGAGAGGCAAGAGAGCAAGGCAAGACTGAGGTGTTACCCTCTTTCAGAGCAGGATGTCTGCAGGTAGAGGTATT
ACCTTCTTTCAGAGCAGAGGTGTCGGCAGTTAGAGGTGTTACCCTCTTTCAGGGCAGGGGTGTAGCCAGATAGAGGTTTTACTTGCCTG
CTCTATATGAAACCCTCCTATCTGAGTGGTGGTTTGCCATTGTACTTCCAGCCTGACTCTTGAGATGTTGATCACACTACTAGGGAGTG
TCAGGAACAGTGCAAATCCATGAACCCTGGTGAAGGTCCAAGTGTGCCAGCAATAGTGGTGAATCAAAGAGGTTTTCATGTATCCTGTC
CCTCCTTTGCATTCCCACACCAATATTCAGAGGGCTAAGAGTGAACTCCACAGTCTTCTGCATCTTGGCTGTCTCTCCAAAGCTTACAT
CACTTGCTCAGGCTGTCTAAACTTGCCTTCTTATCTGCAAGTTAGGGTACTACTAGTGCCAACCCTGCCTATCTCAGGGTCATCAGAAA
GCCCAGATGTTCTCCATAGATAAGAGGGAAGGACAAANNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNCAAGACGAGA
CCAGTGTCATGAGTCCTTCCTATGCCATTAGCTATAATCCCGTTTAGCAGGCTTGATCTGTGCTTCCTGGTTGCACAAATAAATAATAA
AGAACAGTAAACTGACTCCCTGTCATCCTTGAGAGGACATACATTATTAAACCAATATACTATTTCGTCTCTCTCCCTCCCTCCCTCCT
TTCCTTCTTTATAATTTGACTTGACTCAAGAAGAGTAACCAAGTAGCCTTTTTTTTTTTTTTTCTTTAAATGGAAAAGACTTTCAGGAT
GACAAGGGGACTCTTGCTCCTCCTTTTCACACCTAGCATGAGGCCTTTCTCACAATATGCCTCACCAGTGCCAACAAACAAGCAGCACT
CTCTGTAGCTGTACCTGTGCTCAGATCGGTGATTACAGCCATGGCGCATTAAAGCCAGTTAGAGAGAGAAAAATAACCGAGCCGAGTGT
TGACATCGTGCCGTGGAAATGTGAATACACTTTGGGGACGCAATGGCAGACCCCGAGTTCAGGGCACCACCACCTTACCCCAAACCATT
GGAACCAGGTGGTGCATGTTACTTGAGCTTGCAAAGGGCTGGTTCTTTCCCCGGTCATATCCGGTGCCACTACTCACCCCACATGGCTA
TTGTGGCGATTTCTCCCTCCTCGGCTGTAGAGACAGAATATGCACATAGACTTAGATGAGCACGTGCTCTGGGAAATGAACAAGCATGC
GAAGTAAGGCGTTATTTAACTCTTACTACCCAGTCACATATCACTTTCCTACCCTATGTCCTCCAGTCATTCTCGAAAAGGGCACAGTC
GTGGACCCATGATGGGCTGACATTGTGAGGAAGGTGGGAAACACAAGAGACACGGGCTATTGGTGCAGCTGCTTGAAGGAACTCCTCCT
GTCCGCTGTTAATGACCAGCATCAGGGCTCGAGAGCACTCACCACCCTGCGAGCATGACTCCCCTCCACCTGCTGAGAATGCCTTTCGA
GCATGCGCTCTACCCGCCCGCAGAGAACGCCTGCCAACCTGCCTCATCCTCCACTTGCTCCTTGCTTACTCAGCTCAACTCTGCCTTCA
AATCTCAGTCCGCATGAGTTAAACACACGGAAGCTTTCTCTCACTTCCCAACCGTGCCAGACACTGTTATTAGACATTCTGTTTGATGA
GTTCTCCAGTTAGGTTTTACCTGTATTGTTGAAGTTATCCAGTTAGCGTCAGTCCCACTGTTTAATTCCGTGAGAGAAAGAAATTGGGC
TTGTTCTCAACGACGTCTACCCCCCACTCTCTGACCTTCTACAGTGTGCCGCTCTTAGAAAGGGGCGTAACAAACTAAACTGGTAAACT
TTAGTACTCAGTGAAATAATGGGCTAGATAAGAAACAATTTTCTGAGATTGTTGATATCATCAGGAACCAAGTGTTGTCTTAACTCCTT
TAATCTTAAAACAGTCCGGTGATACTTCGTCATTAATTTGATTTTTGAAGGTGGAGAAGCTGACACACGTCACTCCACGACAGCTGGGG
GATTTGACCCTGCCTTTGACAGATCTTAACCTAACCATGACCTGCTGTGGTAAACAGCAAGGTTGGGTTCACAACCACAGCCAGGCCCT
GTTAGAACCCTGAGGATGTTCGAAAGAAAAAATCAGCAGAAAAGAATGATTTTAGGTATGATGTCTTACATTTATGCTCCTTCTCCTTT
CTGTCAGAGTACGTTAGCCGAATTCACCCAGAGAGCAGCCTGGCTTTTGAAACTGTGATTCTGAGGAAGTTCTAGGCCACTCATCTCTT
GTTACCCCTTTGAAGAAACAGCAAATCCTTTTCCACAGTACGTCAGGGCTATCTGACCTTAAAAAAAAAAAAAACTACGTGGCTGACTA
AGAGCCCTATACCTGAGCCCATGACTTGTAGTTTTTATTCTTAAAGGACCTCTCACCCATGCACCCAGAATTCTAGAAGACTTTTCCTT
CATCACGCAACCCCAAGGAAGTCTGATTGTCCCCAACAAGGGCAGCTGACACTAGAAATAACATAGTATTTTGTAGAAAAATAAACATA
GCAAAAGAAAGAAAATAAATGTATTTGTAGAAAACTAACAAAACACTGCAATGTAGGAAAAAAGAAAAACCTTCCCCTTGGAATTGATG
TGCCCCAAGTATCTTCCAGCCACTTCCTTCAAATGGACCTATTAGACATCCTCTAAGTGAGGCACCATCTGACACTCTGAGATAGCCCC
TGAGGGTTGTCCTCTCCTCTGCTGACTACTCTTGTGAAGTGTTTCTGTACTTGAATGTATATATTCCTCATTAAACAGCCCCTTGACAT
TACTCAACAGGTGAAGAGACCAAGCAAGGTCACACAGACTGGAACTGCCTCTCCCCCCCAGCCAGCCTACCCTGTCTCTGAGGTCCACA
CTCTTCCCATTCCGAGCAGCTGACTATCCAGGATGCCTCCTTTGGCTTCTTACCTGTQCTTGA3T3CTTACAATTAAGAGTCTCTGTTC
AGATTCTACTCTCAGAAAGACCTTCCTAGTAGACAGTTCCCCTCTATAAACTTTATCTTCAAGAGACGTAATACTTCCATTCCCTTCCC
ACAATAAGTTTCTACATTGGTGTTGAATACCTCTTGGCCACTATGGCATGATAAATGATGCTTCCAACAAATCGTTGTCAAGGTTTGCC
TGTGCCGCACAGCTGTCCCCACAAGTAAGGCTTTCTTTCTCTCAGATGGCATCTTGGAAAGTACAGAAAGCCCAGGGCCCTCCCTGTTT
TCATTCTGTGGTCTGTGTAAAAACCAAATGCTACCATCCCGACTACACTCTTTCAGATGGATCAAACAGCACATTGGTCACTGGCAGAT
CTTTCAGGTCCCTCACCTCAGAGTCCTCCCAGTTCCTCATTCTTGCCGCCCTTCAACCATGAAGTTAGTCGTAAAAGAAACACTAAGTG
CTTCTTGAATGCACCTCCCACCTCTTTCCATCAGCGCCAGTGACTAACTAGCCTGTCAGCGCAGCTTCAGAAGGCTCGGATTCCAAACT
CTCAGGCCTTCTCCCATGAGGCCTTTCTGCTTAAGTGGAAAGCCTGCCTGATTTCACCCAAGTGAAGCGCTTCCTTCAGCCTTCCTGTG
CTGTGTTCCTGTGCACACTGTCAGTTAAAAGTGGGATCACCTCACTCGCTGCCTCTGCCCATCATTAGCAGACCGATGTGATCAGCCAT
CCAGAGCTGGAATCAGCCATCCAGGGACTTTGTAGGAAGAGCATCATCAGCCTTGGAGCAAAACCACAACTTGGTATTCATTGTCCTAC
ACTTAAATGGGGAAGTATTTGGCCTGTGAAGAAACACGACTTGCTAAATTCACTTCATTTTAATATCAGAGACAGTTTTAAGTTCTGTG
CCTAAGAAAGCAACTCATGCTTTGGGACAGTCAAGGTTAAAGTTAGTACAAAACCTTTCTCTGAATTAGGTCACTCTGTAACTATGGGA
AGGCAGTCCTGCTGATCGCCTCCCCGCTCTGGAAGAAGTATGCCCTGGTCTGTTTTATGTTGTGTTAATTGACAAGCAAATTACACAGG
ATTAAAGTTTGCTTGGGCTGGGCTTTGAAGCCTGGGAGGGTTCAGGTTGTTGGTCAGCCATATCTGACAGGAACTTTGCACCCCAGGAC
AAGACAGGAAATTACATAACAAGAGAGGCCATCTTGGCTGAAGTATCTCTTCCTCTTTGTATAGCTCACAGCTCCGTAGATGACGGCTG
GCTCTTATGGTGTATTCGATTTTACCTCCTAAAGATCAACTTCCAGACACCATCACTGGATTACATTTCTAGCAACTCAACGTCTCATG
AGTTTCGGGGTGGGTTCAGAAGCAACTCATTATAATGTTGTATCAGGGCTCAGGCACCCAGCCCTCCTTGGGGAGTCTAGAGAGCTGGC
TCGCTGGCCAGAAGGGAGACCGTTCGTTGTTCAGGGCAATGGTATCTTTTCTCCATACTCTCTGGGGAGTTACGCGAGCAGTGATCAGC
TTCCCGGCTCTGACCATCAAAGGTTACACTGGAGAAAATCCATATTGAGAGCAAACTCTGTAGAGCCCTGCCTCCGTGGAGTCTGGACC
CCGCTGATAGTAGTGATATTATAAGTGAGCAGGCTGAGGAAGGCTGTGTGTCTTTACAGGAACCTCCTTTAGCAGATGCAGGCTCTTCA
CGTTCACCTTGACATTTATCCAGAGAGCCGACGTACCTCATGCATTTCTAACAATAGGTGGGATTTATGGCAGGTTCTGACTTAATCGA
ATTCAGAGGGGAGTAATGCCTCCGAGCTACTCATGGTGTGACTCTCATGCTGTGATTTCCACGGTGTGATTCTCATGGTGTGGTTCTCA
TGTTGCCCCATCTTGACGAAAGTTCTACACTGTAGAGGTCGGGATGGTCAACTAGACAAGAAAAGACTTGGTCATCTCCTCTCTCCTGT
CCTGTCCATTGCTGTTCTTACCACGTTACAAATACACAAGTGAAATGATCGCCTCACATGGAAAACTTAACTTTGTAAAACAAGAGCTA
AACAACCAAATGTGAAACCTACAAATTTTTAGTCAGAAAGTTTGCTTTTTTTATTTAAGAAACTCACAGACTCCTGAACATATACAGTA
TAATGTCAGAACCCATGCCTGGGGAAACTACTGAAGAGTAGTGGGTGTCCTGTTAGGTGATGTGCACCAAGCAATGGGCTTATGTCATT
AAAAACAGCCTCCAGAGCCAAGCCCCTGAACAACACTACCTTTCTGGTAATCACATGACAAGGAGTTAGGACTCAGTCTTTGTTTGTCT
CTCTTACTTCATCAGTCACCCATTCCCTTCTCGTGCATGGTTTGCCCAGTTTTCATCACTGAAAATCGTGACAAGGTTTCAGAGAAGAA
ACAGTGTTGGTTAGTAAGGACCCTTGTGACAGTCCTACCTAATGAGGCATACTCTGTTTTCAGTGACTTGTTTCCTGGACATATAAACA
ACCCCCACTACCATTTTCATTTATTTCCATCCTGTCATGTCTTCATTGTATATTTTTTAAGTTGAGCTATAGTCAAATATGTATATATC
TGCAAAATGTTGTATTTATGCTGATTGGTTCCATGTTACAGTCAGGTCAGATGTACTAACTGTTGAAAATGTCCTTCTCATATAGATTT
GCAACTATGTGGGGCCTGCAAAGGTTATCGTTCAGTTGGTCACAAATGGAAAAAACATCCACCTGCACGCCCACAGCCTGGTGGGCAAG
CACTGTGAGGACGGCGTATGCACCGTAACAGCAGGACCCAAGGACATGGTGGTTGCCTAAGTGGGCTGTGTGGTATGGAGGGAGCGGGA
ACACGCGGGTGTGAAATCCATAGGCCCCTTTCCTTGTGGACCTGTGCAAAGCCTGAGAGGGTCTCACACACGCGCAAGCAGATAAAGCC
ACGGGAATGTGTGTCCTCTCCTTTGAACTGCTAAGGAAATAGAGGTGACCAGATATCTGTCCTGCCTGAGACAATCCTTGCATCTGTAG
TCTTGCCCTCTCAAGGATGGGTTTGGACAATGTTATAATTATAAGCCCTTGACCTAAGAGGCATTTGTGCCACTTTCAATTATATTTTT
CTTCTCCTGGAATTAAAGATGAAGAAAACAATGAAACAACTACCTTAACTATATAGAAATGATTACCATGGAGATGCTTTTTGCTTCCT
TCAAACAGCATCTCCACTATAGTTTAAGCTTACCTTGAACTCACATAGATCCTGTTGTCTCTACCTCCCAAGTGTTTGGGTGCTGGGAT
TAGAGATGTGTACCCCCCCACACACACACAGAGCTCTGATTTTTGCCTATTTGGGATTTTCTTAATAGAGTTCTAGTGGGAACATTTGA
GACATCAAGCAGATAAGTAAATATTGTTTCAACTCTGCGCTACTTATAAAATGTGTGCTTTTTAAGTCAACAAGACATGCAAATTGGTT
TCCGCACTCATTTGATCTGGAACATAAGTAGAAATTAGTGTATTCTTTCAAAGTCAGTCAAATACAACAATTTTTAGTGAATGAAATTT
TATAGAAGTAATCTTACTTCAAAAGAATCACAAAATATTTAATAAGACATAATTATGGGCTGAGAGAGGATGAGTTTAGTTTGCAAGTA
TGATGGCCCAAGTTCAAACCCCTGGCCTCTCATAGCAGCGCTTTTCACAGCACAGGCATTTATCCTCCCAGCTCCTGCAGGGGAGCAAG
AGGCAAAGACAGGAGAATCTGGAATCTCATGCAGTAGCTGGCCTGGTGTGGTGGACAGACACCAAACAGATCCCATGTCAAACCTGAAG
TTCTCCTCACACCTCCATATGCACAGCACATCACATAGTCAGCTGCGTTCGCACACATAAAATAAGGGCACTCACTATGCTAAGGTTTT
ATATTTCATTATCCTGAAACACTAAAATAGCAGACAATGTAGTAACTTAAACAGCAAAAAATGATGTAAGCATTCAGTCATCTCAAATA
CAGATAACTCCTATTTTCCAGACTCTTAAAAAGCAAAGGGGATGATTCAGTGGGTAAGTGCACTTTCCACCAAATCTGAGACCCTGAGT
CCAACTCCTGTAAGCCACATGGCAGGAGAGAACCAACTTCCACAAATTATTCTCCAACCTCCACATGCAACAAAGGTATAAGTGCACAC
ACACACCATGTAATATAATTTTTAAATAAAAAAAAGTTAACTAAAATATTTTTAAAGCCAGAAGGTAAGAACTGGGGAGACAGGTCAGT
GGGTAAACTATTTGCAATGCAACTTCAGGGCCTGAGTTCTGAGCTCCACCGCCCACATGAAAAATCCACGTGTTAAAAAAACCAAACTG
ATAAATAAAATAAAACCGGGAAAGGGCAGAGCACAGCGGTGCATGCCTTTAATCCTAGCACTCACGAGGCAGAGTCTAAGGCCAGCCTG
GTCTACAGAGTGAGTTCCAGGACCGCCAGGGCTACACAGAGAGAGTCTGTCTCACTGCCCCCCAAAGCCACGTGTAGTGATGCACCTGG
AACCCCAATGTCTCGCCACAGGGATGGAGCAGATCCGCGGGGCTCACAAGCCAGTCAGAAAACACCCCCCAGTCCATGACCCTGTCTCA
AAAACCCGAGAAGTGACTGAGGAAAATTCCCTCCCTGTCTTCTCTGTGGTGCATGCAGATACATTGTTGTTAATTTTATTTAATTGTGT
ACATCATCATTCAGAGTTCTCCAGTCTTAGATCAAGTAGTGTCTGGGAACCCCAGTGAATGTCTCAACTTTTTTTAGGAGATTGCTTAG
CCAGCCTTGATGAAGTCTGACTCTGTTGCCCTGCAATCTTGGGAGGTTCAGCTTTCAAAGTCCCAGGCATCATTTCTTCATTTGACGAC
CAAGATGCTCAAGTGTGAGAAGCTCCCAGAGAGAGGCCCCTTCCCTTTAAGTCATTCATCTCTGGTGCTTTTTGACGTTAGGGAGAAAA
CTATCCCAATTGGAGGAAGCTCAAGTTAGTTCTCGTGGACTTCAGCTTGTCTCGGCATCTCCTCGCTGCCCTGCTTCTCAATCTGCCGA
AACTGACATGCAGGGTGACTGTCAGTTTTAGACAACTCATCGGTTAGTGTATTCCCTGATTGATTGCCCACATCATACCCAATCGTTTT
GAACCATTTCCCTTCTTTTCGTCTTCCTTAATCTGTCTTACAATCATATGTTCTTAAGCAAATCACAGGTCAACTTTTCTATCAGTATT
TCTGTCAGTAATATTTATTATTATAAAACTTCCAGAGTCTTGCTTCTCTGTTGGGTTTAAATAATCTTTCATGATTTAGAAGAAACTAT
GGTGGCTGTGTCTGCTGCTCTGAGGTAGACACAAGGCCTTGGGTTGCTCTCCCCTGCAGCACTACCGCCACTAAAAACACTAATGGCTG
TTTATGCTGCACCCTAGGTTGCAGCGATGGACGTAAATTCAGTACCTGTAACCACAGCCGAGACCTCCCTCGGTGTCTTTTAGTGTAGT
GGGGCACGCGCCTCTAGAGTTCCCACTCTCCATTACTCATGATTTAGCAAACATTTTCGACATCCGATTCCTGACTACCCTCCCTGTGG
TTTGATCTACTCATAGCTGGGTGGGATTTTCTAAGGTGAGGAAACTTTCTCCCACACATGCAGTCCAGGAGATGCCGCCTCAGTGATAG
GGGGTAACCACACTAGCTTTTCCCACCCAACAATCTCTCACTGTAGAATGTCTGCAGTCGCTTTCCCCCTTGCCATTTAGCTGACAGAA
TTCTGCTCATGGTATTTTCCCCAGACACCATTTTCTTCGTGATTCCCTTGTCTCTGTGTCTCTCAGTTTCTAACACGCTCCTACACCTT
TTGCCTTTTCTTCTCTTTCTCTTGGATGGAAAAGCAGCGTGCTAGCAGCCGGGAAGTCAGAGCTCCTGTATGCTTCTGTCACTCTGCAC
GGCAGAAGGGATGGAGGCCAAGTTGCATCACTTTCTAAAGACTTGAATTCCTTTCCTTACAAAATCACAAACACACAGGCTCTGTCTAT
CTCCAGGCACTCAGTGTTTAACCCTCTAGACCAAGCCCACATTAGCTAATGTTATAAAACAGCAGGAGGAAACTTGCCACTATCTCCTC
ATGGTATGACATCGGTTATTTTATTTAGTTCTGACTTGTAGGCCAGAAGTTTAGGAAGGCAGCCAAAGCTCTTTCTTACACTTGTCAAT
ACTAGAAATAGAACCAAGATAACATTTTCCAATACTACAGATTAGACTGAATATAGATTCACAAACAAGGCACCAGTCCAAACTATCAA
CATATGTAAAAATGACCCCAAGAAGACCAAGCTTTGAACACATCAAAACCAAACCGACCCCCTTTGTTTACAGTGTCTTTCACACTTTC
TTCTCTGTTTGAATTCTTAATAACTTTAGTTTCTATTTTTCCAAATTATTTTATCAGTATTAGAACTATCATCTCTTGGGGTTTGATTA
TGGATTTGATTGTTTTAATATAGGGTCATTAACTTCAGTTAGCTTTCATTAGACCTCTTGTCTCTTATGCACTTAGTTAGACTTCACTG
CCTTTCTTAATAAAAAAAAGAAGAAGAATCCTGTTTAGTATTCTATTTTGACCAGAATGTTAATATTTTTACCAACACTCTAGTTCTCT
CAAATTTTTTGTTCAGTGGAAAATGTCTGTGCATTTCTACTTTGCTGTCCGCCACGTCCACCAAAAGTCACCACACCCTGTTTGCTTAC
TGGCAACCTGGCTTCAAAGAGATGTACAGTTCTTTTTATTTCCCCCTCCCAACTAATTTATGTACCTTATTGCAGTTGGTAAGCAGGAG
AGCAGAGATTCTCAACCTGTCGGTTGCAACCCCTTTGCCCCTCAAATGGTCTTTTCACAGAGCATGCCTATGGAGATTTATATTAACCT
TTATAACAGTGGCAAAATTACAGTTATGAAGCAGCAATGGAATAATTTTATGGTTGGAGGTCACCACAGCATGAGGAACTGTTAAAGTG
TTGAAGCATGAGGAAGGCTCAGAGCCACTGCCCTGGATGATAGTAGATATGAGAACACACAGGAGAGCCTTTCGCCTCCCTCAGGAGAA
CTGCTACCTTAAATTACTGCTGGACAGATCCAGTGTAATTGTGTAGCTAAACACTAGCCCACAGGAGAGGCACTGCCTAGAGAAGGTTG
GACAAATATGGACTTAGGTTCAAGGTGTGCCGTACCAAGTTGGTGTATGCACTGTGAGACGAGAGCTGCTTTTGAACACTGAAATCAAG
GTCTGAAACAACAAAGGCCAAAGAGGGGCCACTGTGAGCCCCAACCCAGTGTTAGACACATCAAAATTTGAGCAACAGCTTCCAAATGA
TGATGAACATGAAACATCTGACCTACTTCAGCATGTTTATTGATGGCTACAGGTTTGACTCAGCCTGGGTCACTTCATAGAAAACTGAT
GCCAGCCTCCATCCCATAGAGTGGGATTTCACAGAGCAGTGAAAAGCCAGGCCCTCCAGCCTCAGCATTCCTCTTGAGTTAATGGAAAG
GTTTGTTTGAAAAGTCCTGATTCCGTAGGGTTGGATGGAGCCCCAGATTTTTAGTAAATTCTAAAACAACACTGATGTTGCTAGTTAAA
GACTCCCATGTTGTGAATATGAGAGGAAGAACCAAGGTTGTGGTTTCAGCCCTTTTATTAGTGGTTGTAGCCTTTCTGTTTTTAAAATG
TCATTGCTGCAACACTCCTCCTCTCCAGTGTTCATTTCTGTGAATTTTGATAATCATGTGGCTACTCCTGATCTGAGGCTGATGAGTTC
TCTTTGGTTTTATGTCTGAAAGTCGTCCCTTCATCTTCTCTGTTCAAAGGTGTTTTTTGAAGAGCAGAGGATTCTAAGTTGACAATATG
TACTTTCATGTGCTGGTTTTATGTTGATTCACCGTCTCAGGTCTTGACCTGTTTCCAGCAAGAGCTCTCTGTACCTCTGCCTCGGCTCT
ACTGTTCCCAGAGTGTGATTGTTTCTCTAGCAGCGTCTTCACTTCTCTTTAGCACTCCTAAGCAGTGTGAGTTTTGATGGGTTTGAGGG
TAGCTTTCTTTTTAAGCTTGAGCTTCTTAGTCGTCATAGATCCTTGGATTTACATTTCTTATTAAATCTTAAAGCTTTTCAACTGTTAA
GTTTTCTGTCACTGTCACTGCCCCTGCTCCCCATTCCTGTTCTCTGTCACTTTCTCTCTTCCAGACTAGGGTTGAGTTGGCACAGTGAC
TAAGAGAATACCTTCTGAATAGTCAATCTGATGCCTCATCTATTCCGAGGCTTCCTGTTTATGAGAATAGTAGCTCTTTCCCTTAGCCT
AACAGGGCTTTCTCACAAATGTTTCCTTTGGATTTTTGAAAATCTGTGATGCTCTGGATATAAATCCTAATCTTCTTAACCTCAAGTCT
TTGCAACTGCAGAGCCCTAGCTCGCTCCCTGGGTTCTCCTTCCCATCCTGCAACCTAAGCATTAAGCCAGGTAATTGTAAGAGTACCTT
TCTATTTTTTTTTCTCGGATGACTGTTGTGTGTCACCTATCTCCAGAGCATGACAATCGTTTTCTCGTGTGTTTTGGCTATTTATTCT
AGAGGAACCGTCTAGGTCTATATTACTCCACCATGGCCTGTGGTGGAACACTGGTGTGTTGTTTACTAGGCTAAACCCTTCCTTCATGA
TTCATATAATAAATAAGCATGATGCCTTTCTCCAGATGTGAACCTGGGTTACGTGTAAGAAGTGCAGCCGAGGTGATGGTCACTGATGT
TCCCACGACATGAGCCTGGCCTCCTAGCGGGGGAGCAGTCTGCACAAGAGTGAGACCCAAAAGGGCAGACACAAGATCTGTTAGACACA
AGGACCTCCAAGCACATTAGCACGTTGGCTGCATAATGTTTGTGATGCTCCCTCCGTTCAAGCTTTCCTGTGGTCAGCACGCACCTGCT
GAGGACCCACATGTCAGCCTGCATTCTAAGACCTGGGGATCTCTCTCCTCAAGGAGCTGTGGAAACACAGCATGTTGACTTCTCTGTGT
CCCTGTAACTCACTCCACATCTGGCCTTCTGTGTGTGTCTGTTTCCTTCTGCCTTCACCTCTTCTCCTCCCTTTCTCCAGTTTATATGA
CCTTTGCACAGTAGCTTGTTCTCATGTGTGCAGGCAACTTCTCAGATCTCTTTTATTGTCATTTTATATTACATTTACCGTGTGTGTGT
GTGTGTGTCTGTGTGTGTGTGTGTGTATGTGTATTTCTGCTTGTCGGGGGAGCCTATCTGTTCTCTCTCTCTCTCTCTCTCTCTCTCTC
TCTCTCTCTCTCTCTCTGTGTGTGTGTGTGTGTGTGTGTGTGTCTGTGTGTGTGTGTGTGTGTCAGTGTCAAAGCCCAGAACAACTTGT
CCAAGCCGCTTCTCTTCCCCTCCTATGTGGGTTCTAGGGATTGAACTCATCTCACCAAGCTTGGCAGCTAGTGTTCTTACTGACTTAGC
CATCTCACTGACCTGTATCATTTTCTTCCTTCTACTAATATTTATTTTACTTTTAGAGTAAAAGTCCTTACAAACATCTATATGGCTCT
GAGAGACCCAGGCCCTGCACTTTTCTCTTTGAGTTCATCTGTCCCCTCTGACTCTGCTCCTTACCCTCAGCCCATGTGCCACCTTTCTG
CCACTGTCAAAAACAGCAGAGTGCCCCTGCCATGCGGTCTTCACATTCACCAGAGTCGTCCTGCCATGAGGTCCTCACACTCACCTGTC
TGCAGCTCTGATACTTAGAATCCACTTCTTCTGACCTCCCTCAGGACTGGCTCAGCTGTGATCTCAGGGTCCTGTCTCGGTTCTCCCTG
TCTGGCTCTCCTACCCTCTCACCCCTCCCAGGCCTGTCTTCTTTTTCTTGTTTACATGTATTTCTCTTTTAAGTTACACTGTAAAATCT
ATCATCGTGTACCCTAGCATGTGCATTGTGTGAGAGCACAGTGAAGCCACACAAGCCTTCCCTAGTAAAGCATACGTACAAGGGCACCC
GTACTCTGTGACTCCTGAGTTGACATCTAATATCTTCTGAGACGTAAGGTGATTCAAGTTTTCCTCCAACTCTTTTAGTAACCCACTTT
AACCTGGTCTGACAAATCCACTTCTGTGATGAGTCTGGTTACAGGCCTCTCTGCACAAATGTCAATGAGCTGAGTCCATGGTCCACCCC
AGCCCTGCCAACAGTGTCTAGTGACGTACACCATATGCCATATATCACCCTCATAAAGTCCCAAATTCTGATGTTCATCTGACTTTGGT
CTGTGTAAGATGCTGAGTGCTTGTGTACTTTTTTCGGTGTGCTGTCTGCTGACCTGAAGCCCGCCCCCCGGCCATCACCTGCCTGACTT
CTCCTCTCACTGTTTTTCTCCAGCTTTGCAAACCTGGGAATACTTCATCTGACTAAGAAAAAGGTATTTGAAACACTGGAAGCACGGAT
GACAGAGGCGTGTATTAGGGGCTATAATCCTGGACTTCTGGTGCATTCTGACCTTGCCTATCTACAAGCAGAAGGCGGAGGAGACCGGC
AACTCACAGGTGAGCCAGCTGACCCAGCCTTAATTAAGGCTTCTATGTTCCAGGATGACTGCAGCCATCAGCGTGCCAGGCTATCAGAG
GGAAGGGGATCAGTGCAGCCCGGTCTTTCCTTTCTGTCCTGGTTCACCGTGGAGAAGCCGAGCCCACACACAAGACTCTGCATGCTTGT
TCTCACCTATCTCTCCCCACCCGGCGGTGCAGAGACGTGCATTTCCCTGTACTCCTCGCATTCTATQTCTGCCTTAACTCTTACCAACC
GTAGGAGAGTAGATGGGTGAGCATCGCACAAGACAATATTTACAATACTCCAGACAAAACGCAGCTTACCTTTGATGGGTTTTTACCCA
ATTTAATAACACCATTGTGTAAAATTTTACTAACAGTTTAAAGAATTTCAGGCCAAAGTAAACCAAGAAAAAAAGAAGACTCAGTTATT
AAAATTAAAGGTGAAAATCAGGGAAACATTACCAGAGGTAATGGTCAAATCCAAAGAATCATTAGGGCTTTGAAAATATGTATTACAGC
AAATTAGAATATCTCACAGAAATGGGTAAGTTCACAGGTACATCTGACCTGCCAGTGTTAAACTAAGAAGATAGAAACAACTTACACAG
ACCTAAGACAAGCAATAAAATATCTCCCAACTGAAAAATACCCGGTCCTAAATGGAATCATGGCTGAATTCTGTCAGATATTTGAAGAG
ATAACGTAAATCCTCCTTAAACTGTTCCATAAAGTAGAAAGGGGAAGAATATCAACAAACTTATTTTCCAAAGCCAGCATTACTTTTAT
ACCCAAACCAGGTAAAAGAAGAAAAGAAAAGAAAAGAAAAGAAAAGAAAGAAAGAAAGAAAAGAAAAGAAAAGGAAAGGAAGGAAAGGA
TAAGCCGATTCATACATTCCCAATAAAATAATAGATGAAATCATGATATACAAAACCGCAAACGGAAACACATCAAGAGCCCTATCCAC
AGACAATATTCTCCCACACACAACACCCCACGGCTGCACTCCAGAGGCTTAGCTCTGATTCACCATCAGCCCTCCCTCATCACCACACA
GACCCCCTCGTTTTCCTATATCGATACTCACTCTCTCCACAAACAACTCACCAAACCCCACCCCCGACCACAGTCACACCCATCCCCCA
GTAGATTGAATGCAATCCACAATGCCAATACCGTTCCTCCTAGAAAATAAAGTTCATTGGGACGACAAAAGGTCTCAAGGTAGCCAAAG
AAACACATCTCAATCAATCATCCAAACCCCTCCCATGTTCCTCCGAGTTAACACAGTTACTACCACAAAAATATCCCCAACTCTCCTCT
CTACATTCAATGCAAATCCACAXTCCCAATACCGTTCCTCCTACATAAAACTTCACTTGCCACCACTAAACGTCTCAGGTAGCCAAAAC
CTATACCATACTAACAATATTTCACACGTATCACACCTGCTCATATCAGAGGTATCATAGCTCCTCATTCCACCTCACACTACAGACCC
ATCCTAACAAAAAAAGCATGCAACTCCCCTCACACACCTCTATCACCACAACAGCAGACTCACAAAACCAAACACAAAAATCTACCCAC
CTCATACCCCAGAAACATGCCAAAATCTCCATTAGACAAAACACCCCCTCCACAACAACTCATCCCGCTCGGGAACCCCCTAGCACATA
TAACGAAGCCAACCAGATCCCACCCTCCTACCCTACCAACCAATTCAACACCCAGCACAGATCTTAAATGGACCTAAAATCCTCTCAAA
CAGCTAAAGGAAAACATGCACAGAAAAGCTTCCTCATCTACCCAACCCCTTTCCGAACCCCTCCAGCAACTCAGGTAATAAAAATCCAC
AACTAGCAACTCACATTCAAACCCCTCACAGCAGAGGAAACAGTTTCCTGAGTTGCAGAGACCACACCCTTCACTCCCCCAAAGAATTC
TTGCTAGCTATGTAGCTGACAGATTAGTATCTAGGTTCTATAAAAACTCAAAAAAAATTAAATTCTGGAAACAAACAACCCAGTAAATA
AATCCCCTCACAACAAACACACCCTTCTCCAACAACAGTCCAAACACCCAACAAATCTGATAACCATCCTTCACACCCTTACTCTTTAA
AGTTCCTCTGAGGTCCCACCTCACCCTCCTCACAAGTCATTAACAAAACAACTCCACCACCCTGCCAACTACCTCACCTCCTACACTAC
TTACCTCTTCTCTAACAACTCCTCCCTTTCATTCCCACCACTCCCTACAAACCCCTCCACTCCCACATCACTTCAACGTCCTCCCCACA
TCTCTGTACCACATCTCTTACCACCTAACCCATACAAAGCCTGCTGCCCTGAATTCAGTCTCCACCACCCACATCCTACAACCACACAA
CCCCTCCCCTCACCTCCTCCTCTACCCTACACACCTGTCTTATCACCAACAATCCAACAATCTTATAAAAACAAAATCAAAAATGCACA
TTTACAATAACATCAACTAAAACTCCATACACACAGCTATCCACTCCTGCGCATATTTACAAAACTCTAACTCCACCTACTATAACATA
CATATTTCCCTCCACATATGTGTTTATTTCACCCATATTCAACACCTACATCAAACAACCACCCTACCTCTCCATTACCAGATCATCCA
TTAAGAAAGCATGATATACATCAGTAATGGAGTTTTTTTTTCAGCCATTAAAAAAAAAAAAACAAAAAACAAAGAACCCCTCCACAGAT
CCCTCAGCAGTTAACACCACTCACTCCTCTTCCACACCTCCTCACTTCAAATCCCACCAACCACATGCTCCCTCACAACCATCTCTCAT
CCAACTCCATACCCTCTTCTCTTCTGTCTGAACACACCTATACTCTACTCACATACATATAAAAATAAAATCTTTATTTTTAAAAAAAA
GAAGGCAACAAAAACAAAACAAACATTTCCACCCCAGTGTATCCAACTGGAGATCTCTCTATCAACACAAATAACCCACAACAAACAAC
ATCAACATCACCTACCGTCTCTCTCGGTTCTACATCCTACCTTTTATATACATCTACATAACATATTTTGCATATGCGTCCATACCATA
TGAAACTAACACCAACCTACTCCCTTCCTAGCCAGGGGACGAGTCCATATCACCACCGGACAACCCCCCTTAGCTACCCTCACAGTCCT
TCTTCTAGCATGAAAATGTTTTCACCCACTTATCCCGATCACCACTCACTATATCCCAACAAAGACTTTAAGCAACTCTTTCTACACCA
AAACAATTGCTCTTTCACCCCATTTCCTGTCAAATCTTCACACTCTCACCTCCCTCCCCATCACCACACTGTTCTCTCTGACCCTAGAC
ACAGAGAACGACATCATCCCCCACCCACCCGTCCACCAGACCAACCAGATGGACCTGAGCGTCCTCCCCCTCATGTTCACACCCTTCCT
CCCTGACAGCACTGGCAGCTTCACTCCCACACTCCACCCTCTCGTGTCACACCCCATCTATGATAGCAGTGAGTACTCCTTCCCAACAT
GGCACACTTTCATCTACCTCCATCCACGGCAGCGCCACCATGTCACTTACTCCCCACACCTCACCCCTCCAGACTCCCACCTACTCACA
TAGAGCCTCTAGTTTATTCTCTACAAACTTAATACTCCAGTCTACGGTAAAGGGTCAATATCCACTTAACCCTCTTAACAACATCTCTG
CCAACCTAACACGTCTTCCTCAGAGAGTGCATTATATTCCTTACATGTAACACCAGTTTCTTACTATGAACACATGACTGTAACGATAC
ATTGCTGGTGCTCTAAAAAAAATTACCTTAATTTTTGCACCTGGCAGCACGGAAGAAAAATTGATAGAAGACAAGTATCCCTCTTGTCT
CTCTTTATCCCCAGTTTGAAATACATTTATTAAGACAGGAACGTCAAGGTAACTAAATAAATGAATAGTATGTCAAAATCATCTTTCAG
CCTCCACATAAAAACAATGTGCAGGGTTTCCACTACCTTCTACAATTTACTCTGTCCCTCTATTCTTCGATATGCATTGGGCTTACAAA
CAGTTCTTTCACCAACCCTCCAACCCTCACTCAATCCTCATAGAATACGATGCACAACCAAATCCCATCTTCTCCATCCACCCACCCAT
CCCTCTCTCTGATTTAAACAACTCTCAACCCAACTCAAAACTTTATTCAATCGAAAAGAGTCTTACTCAACACACATCACCTACACCTC
TTCCACTTTTTAAACCCCCAACCTTTGCCCCACTCACCACTATCCCCACCTTTCCCCCAACCACACACAATCCTAAATTTACGTTGCGG
GAAAAAAAACCACAACATCTTCACTTTTCTTTCAACAATATCTGTGTGTGTGTCTCTCTCTCTAAATCTCTCTCTATATATATATATAT
ATATATATATATATATATATATATATTAGCTTAAAATGTCATTTTCTTAAAATTTTTAAGAATTTTACAAATGCATAAGTTAGCATGAA
ACAACCCATTCCTCCACAAACATCACACACCTCTCTCCTTCCTCATCACCCTCTCTCTTACACACCCATATCCCACCAATCCCTCCCTC
ACCTGCTCCTTTCTGGGGAACCATCACTCCCCCACACCCAACAACTCCTCCTACTACCTTGCAAGTCTTTCTTCCTCTATTTCTCTCTT
GTTAAAGGCAGTAAGAGTGAGCACCATCATGGAATCTTTGTGAGGCTAATGCTAAGCCAGGGTATGCATTCTCTTCTCCCTTTGAACAG
AACCCCCGAATCCATCCAACCTCAAAATCCTGACAATGGACACAACAGCACGATGTGTGACCCCACCCCACCACATTTACCTTCTCTCT
GACAACGTTCACAAAGGTACGTACACACCTCCTCTCTACATCTACCCTCTCCCTCTCACACTTCTCTCGAAACAGCGCACCACCCCTCT
TCTCCTTTTCCTCTCCCACTCTCAAAAACACAGCGTTCTTCAACACCATTACACCCATTCCAATCACATCCTTCAATACCCCCCTTTCC
ACATCACATCCAGATTCCCTTTTATCAACACCAACAAAATCCCCCACTTTCCCAACCATTTGGGGACTTTTCCCCCACCCATCTTCATA
CACACCTACGTCCGTTATTATTGCGCGTTTTATTTTAATCCACTTCTCCCTTCCTCAAATTCACTCCTCACTTTCAACATATTAACAAA
CAAACCGTCATAACTCTCTTTCACATCCATACAGCAATGCTATGTTTATGGCTCTTCCACTACTCTCACATACACCCTCACAAGTCTAA
AGGGCTCAACCTGATCCTGTCCTCATCACCTCCTCTCTCTCCCTCATTCCTTACATCACCACCGTCTCCCAGCTGATCACATCACCCAC
CACAGTAATAGTCACTCTTTGACAGTGACTGAGAAATGGTGTTTCAAATAATTGGCAACAACCCAAATGTTAGCCAGTAAATCACTAAT
TAGATAGTGGTGTATCTATGTAATGGAACATCATACAGTTACTAGAAAAGAAGGCTCTGTGAATGAACAAAGACCTCAAAATAGAATGC
TAGATAGTGGTGTATCTATGTAATGGAACATCATACAGTTACTGAAAAGAAGGCTCTGTGAATGAACAAAGACCTCAAAATAGAAATGC
GAGACAATAACAACATCACCTCCCTCTACCATCTCAAGTTAGTGCATGACTCCCAACCCGCCACCCACCCACAACTCTCACCATATCCT
ACCTATTGCTCATCCTATATTTGATGACATCTTCATCCTTATAAACATAGAAACACCCAAACTAAGTCTCTCCACTTCCTTATTATCCC
ACCACTCCCCACCACTACCATCTCACACCTCCTCCCCACCCTCACTTACCATCACTCTCTCCCCCCTCTCACCGGCAGCCCTCCTTCTC
TCTCCTTCCACATCCACTCCTTCTCTTCCTCCTCCCACTGATCCTGGATGTTTTCAACCCTATCTCAAAACACAAATCCCTCACTCCCC
TCATCCCCCATCATCCTCTTCTCTTTTACTTTCACCCCCCCACTATAACTCTCCCTATAACGTACTAACTCCCAACTTTCTCCACCTCC
TCCCTTTCCACACACTTTATTTAAGTTCACAGTATACATACACCTCTATACAATCTCCCTCATCCTCACCCACACTACAAAACAAATCT
CCTACACTAATGTTTGGGTCTTTTCCACCCTGCAGTTTGCCATTCTCTTCAAAACCCCAAACTATAACCATCTCAACATTACAAACCCA
GCTTCCGTGTTTGTTCAGCTTCGGAGGAAATCAGACCTGGAAACTAGTGAACCGAAACCCTTTCTCTACTACCCTGAAATCAAAGGTAC
CTCCCTCCTTTACACTCTTCTCCTTCCTCTCAAACACCACTCCCCCCCTCCTCTACTCACCCTCCCAGAACCTGCTCCCTCCATCCCTC
GATGCACCCTCCATACTCTTACCCCCAACCAACCTCATATGCCAACGTCATCCAATTACCCCCACCAATTTATCTTACTATATTCCTCC
CAAACTTATATGAGACTTACACATTTCCCTATTCTTCCCTATTACAAATCTACTATCGACCCAGCAAACATCCCTAAACACTTCCTCTC
ACCCCTCACCTCACCCTCACTCCCAAACCTGTCTGCTCAAACCACACACCTACCTCCCACAACATCTCCCTCGTCCTCCACCACCACCC
GGCTGGGCGGGGAAGAAAGGGAAAGTAAAAAAAAAAAAAAAAAAACTAACCATTTTTTCACCTACTCACAACCCCTTAACAATTAACCA
GAAGGCTTAGCCACTGTCCAGCCCAGAACTATTCAAAACATCCTCACTATACTTTCCTCACCACACCTCACCAAAAAAAACCTCCACTA
TACTTAAAATCCACATTACCATTCTTTTTCACCTCCTAGACATCATATTCTCTCCCCCTTCTACACACTACCCACCTCCTCTACCATTC
AGCTGTAACCCTGCTTCTCAAATGATAAGTACCCTGGGAAAAGCATCCACAAACTGTATCAAATAGTTCAGCAGACACTCCTGGATTCC
TTGGTCTAAAGAATCAGCCAAAGAATTTCTCTTCAGTGGGAAGCCACTTACCACATTATGAAGGTGAATGGAAGTCAAGGCTCTGTTTA
TGAAGACAGATGTTCAGAGGCAGAGGTCTGGCAGCTCGTGTTTGTCCTGGCAGAAATCATTACTGTGAATCTCTCTGGACTCAGGGAAG
GTTGATTTAGGCGAGGGAGGCCAGGTCCACGCACTGAACCGTTTCTCTCGTAGCTGGAGGTTTTTGCTTTGACTCTGATGGCTAGCCCT
GCCTGCTCACTAATCTCCAATGATGTGGTCACAATGTTGCTAGAATTCATGGTGCCTCAGCAGAGTTACAAAGGGTTCTAGCAGCCACA
AGTTAGCATCCAGCAGATACTTCTATGTTGGTCCTCTGGAGACCTGGACCTTGCGTCAGAGAAAGGAAGAGAGACTCAAACAGTCTCAG
ATTGTTCTAGGGTTTCCAGAACTAGAAGTGTATGTATGGAGTGGTTAAGTGCATAAGAGGAACAAAGAGAAGAGTCTGAGAATGAAAGA
GAGCTTAGTGTAACATATTTCAGTTGGGGTCAAGTGCAGGAAGGGGGCCAGTCAAAGGCATGACTCAAAAGTGGGTGGGGGCTCAGCAC
TGAAGAGATCCCGGCCCCACAGAGTTTAAAGTGTAGGGACGCACAGTGCTGCCGTCAACCTCTCTGTACCTCTGTTTCGTAAGTTTTGT
CTTGACGGCCATGGTTCTGAGAAGAGATCCATGAACTTGGAGGCTACACAAGGCCTGGGATCTGATTAAATCAGAAATGTCTGGCTCAG
AGAGAATGGGGCTGGAGAGGATGTCACCAGGTACAGGGTCCTAGGTCCAGGGCCACCAAGGCCCCCAGGTATCGGTCCCTTGAAGGCAA
AGCGTGATGGTGGCCAGGAGTTTCTGGGAGGCCATGTCTCTTTTTGAGGTATGGCCTCCCCATTCCTGGGTCAGGCTTATGGTGAGCTC
TGTGGCTGGCCTACTTCCTTCCTCACCAGCTTGAGGTCTTCTAGATCCTTCTGTCCAGACGCAAACATGATAAATTACTGCTTTACCTG
AAGCCTCTGCCCTCTGAAAACAGGAAAAAAAGATTATTTTTCTAAGAAAAAAAAAAAAAAAAAAACAGTGTATCTCCTGAAATGTGCCT
TTAAAAGAGCGTTGTCATTGCTGATGCAGAATCATGAGAAAAAGAAAATGAACTGAGGTTCCCTGTGCAGGATGTGAGCCTTGAAACGC
TGAGAGTTTGGTTGCCAGGCAACCTCTCCTGCTAGTGTCTGCATCTGAAGAGTACAGTAGACAAGGCCCAGCTTCCTGGGCACTCCACG
CCAGCAGAGTGGGAGTTTTCTAATGCCTGGATTAGAGGATTGGGGTTCCCTGAAGTCCTGGTTACCTCACATCTTCTAGGTCATCATGA
ATACAAATGGTATACTTGAAATACAAATGAGGCTCCACCTACACACAGGATGGCTGAGGTACCTGTTGTGTTCCGTGTTTCTACACTGG
AAACATTTTCTCTGTGAGATCCACAGTTCATCCACTGACTAAAATGAATATAGCTATTGGTTTTGGTATAACTGGTGGGAAACTTTATA
AATATTTCTCCGAGCCATCAGTTAGAAAGTAGACAGTCTCTTCACCTCAGCCTCACTCCTTTCTGGCCGAAAGCCGTTTTCTTCCTAAT
CATCCTGGTGTGCCCAGGGTTGGTTTCCCAGGGACTGGCAAAGTCCACCATGCTCTCCTGCTTTCCCCATCAGCTGCAGTAGACAGTCC
AACATAGTCCAAAGTCCTTGTGCCACACCGTGGGGTGATGGAACACCCACAGAAAACTAGAGAAGTAGGCATCTTTCTGAACATAGACC
ATGATGAAAGCCAGCATTAATCAGTCAGACACAGGACGATTGTTCAGTATTGGCCTATTTCCTGGATAGAAGCACCATGTTGAACTATT
TAATGTAAACACAAAACCACCTTCACTTCTCAGCCCCAAAGACAAAGAAAACAAACAAACAAATATCTGCTAGGATACTCCCAGACCAC
AAAGGAACAGGAGCACCATGTCAGATCTCCCACACAGTAACAGGACTGAGAACATGGTGGCTGAAGATCACACGGGCCATGACAGAACT
GAGGGCACATGGCTGTCGATCACATTGCAGTAACAGAATTAGGGGGGCTGTCTGGGTTAGTAAGGAACACAGACAGTGAAAGACAAGAG
GAACACAGAACTCTGTGAAGAGCACCAAGATAAGTCTCGGAGCACCTGGACCTGGGCAGGCCACTGTCCCAGAGGTCACTCACTGTCCC
AGAGGCCATCTTTATTTTGGAGTCCGAGATCTCAGGAGCCAAAACATCTCTGTACACCCTCTCTGTGTTTCTAGGATCGACATTATATC
TCTTACCACAATAACCAGTAAGGACTCGGTCTCCATTGCCTCATGCCTGCACTGTCTTCCTTAGAGTTTGACTCTAGATGTCCTGTATA
CAGTGATAGATGTAGCTCCTAAAATGCTGTCCTCTATGTCATTCTCTGAGACATTCAGCAAGTCCCTCACATTCCTATCCCTGAGGCTC
CTCAGCATGGGTGACCCCAGTCCTCCTTTAACCTTTATTATCAAAAGCACCCACCCATGTTATCCCTCTGAGAAGCCCTGTGTCTTCCC
AGAATATCATGTTCTTCGGCCATAGTTGTTAGCCTTTATAGAGGCTTGTCATGCTGTAAATCTGTTTTCACGTGATTTCACATGATGGT
ATGATGCATACCACCATAATCCATCCCACTAAATCCCTCTTTGCCTTGAAATTGACGGTCCTTGACAGCAGAGATGTGTGTTATGTTTC
TGTCCATCCCAGCACCTTGCTCAGTGTATGTACGGAATAAATGTAAGCTGGTTATTATAGTTGTCTTAGGTGAACAAGGACAACTGTTC
TGAACGTTAGCTGGGTGTGGTGGTGCACACCTCTAATCCTAGTGTTCACGAGGCAGGCAAATTTTGAGTCTTGTTCAAAAACCAAGCTA
GTCTACATAGTGAGTTCCAGGCAGCTAGAGTTACATGGTGAGACCCTATCTCAAATAAGAAGTTAAAGTGCTTTAAGAAAGAAAAAAAA
TATGTGTATCTTTGGGTTATCTGAATCTTGAGAAGCAATGAGATCTCTCACCATCTTGTTAGCACTGGGAACTTAGGAGGGGGCGCTCA
GGAGAGTTGGGTGTGTGCCGCACTGTGGTCTCTGAGGCGTGCTCTTGGTCTTCTCGTCCATTGGAATATAAACCATGCTTCCTCCTCAA
GTGCCTGTTATTTCCCTTTAATGCATTTAAAAATATCATGAGAATCAATCGTCTCTCTGCGGCTGCTGGTGGAACCCGATCAGTCTTTC
CCCCCTGGTTTCCCTTCCTGTAAAATACAGACAAAGAGGAAGTGCAGGAAACGCCAGAAGCTTATGCCGAAACTTCTCGGACAGCTTCG
GCGGCGGCAGTGGAGCGGGAGCCGGTGGTGGAGGCATGTTCGGTAGTGGCGGTGGCGGAGGGAGTACCGGAAGCCCTGGCCCAGGTACG
AGAAACATCTCTTCTATCTAAAGCCTTTCATGAAGAGGAGGGGGTAGTTTTCTTTCCCAGGTTGCCAACAATGTCTGCCCATGGAGTGA
TGGTGTCATTACTAATTATTAGATTTCAATTAATGGAAAATTTCCTACAGATGTAAAAAATTTTTTGTTCTCAAAGAAATGTATGTCCA
ACTCAAGGCACAGCCACTCCCCACCCACCCACCCATCAAGTTTCACTTTCTGTGGTTTCAGTTACACGCGGTCAGCTTGAGTCTAAAAA
TATTACCATTGTGTTGACAGAGAGAGACCACACACACAAAACTGTCTTCACAGTCTGCTGTTGTGACTGCCCTATTACTCTGTGTTCTT
GGTCTCCTTCTGTCTTTTTACGTTGTGAACTTTACCCAGGTCTGTATGCATCTGCAGTTTTAGGGATACACTGGGGGTCTCGGACGTTC
TCTTTCAGGATAATATGTGTCTAGTCCCGAAAAGTGTAAGAAAAAAAGTCAACTGAAGGGTTTCTGGAAGAAAACCTTTCTCAATCATT
TTTGAGTGTTTCCCTCCTCCCCCTCCCCTTTGCTTTTGGAGAGCAGTTAGTCTATTATTCTGAGATATTAAAAAAAACAACTATCATAC
CCACCGCATGGTAGATGGGTATAATATGCCTACACAGCATACTGTGGGCTGGAGACACTGTACTGTAACAACTGAGTCAACAGAAAAAT
CATGCCAGGGAAGCACTTCAATGGTGTGTAAACTACTGCTCCTAACTGCCACGCATACGGCACAGTCAGTATTTATGTGTTCCTCTCGG
GTCCGGCTATGATCCCCGTGCGTGGATTTCCAGCCATCTAGTTACACTCAGTTTTTAAGCATCGTTGCATCATGCTTGTGGATGCTGAG
TACGCTGCTTTTGCCTCAGATCTAGCTAGCTTAAGCATTTCAATGGAAATACAGTTCTTATAGCAGCATAAGTGTGCTGTTTTAATGAC
TCACACCTGTAGCGTAAGTGACTTGAATGCTGTTTCCTGTCTCCTGTGTAAAATAACTGTGCTGAGCATGGCCTTTCTTTATTCCAGAT
AACATGTAAATGCTTTAAATGTCAGAGAGTGTAGTGCTAAGTGCTGGCTGCCCCTCTAAATTTATCATTCATTTCTCCACTAAAGTATC
ATTATATTACGTATTGGGTTCTTCTCACCAGGGGTCTTCATATTTAACTATTTTTTTCTGTAGGTCTAAGTCTTGGTGTGAGTACTTAC
CGTTAGCTTTTCCCTTCACCTGGAAGAAAGGGACCTGCTCAGCCTTAAGTCCTCCAAGCTGCTAGTCTGGGCACTGCCTGTTTGTTCAG
CTGTCTGTTTCCCACAGTGATTCTGCTAACTCTCGGGCAGAGCCAGCCTGTGTAGGCTTGTGCCTCCCCATGCCACAGCCTGCCTCCCT
GAGTGGGTCCCCATACTGTTAACAATCTCCCTGATGTTCCCTTTGTCTCATTGCTGGTTCAGGGTATGGCTACTCGAACTACGGATTTC
CTCCCTACGGTGGGATTACATTCCATCCCGGAGTCACGAAATCCAACGCAGGGGTCACCCATGGTTTGTCGGATCGGAATACCCAGAGC
ACTGCTCGGGCAACAGGATCATCTCTAAAAGCTTTGGAAAATTACTTTCCAATAAATTAATATTCTCAGGTTATTCTAGGACAGCATAA
GGGACACCCCTGACACGTATTTGCTCATCACATTGTGTGGAAGCAGGACAGAGGCTCTAGACAAAAGATGAGACTTGACTACGCAAACA
CACAAATACCACCTTCACATCTGTCCCTTTACCCCAAGTCCTATCCTTCTGAAATCAGTTTTCATTTTGTAGGGTTGGCCTTTTCCCAC
TCTTGGGCATCCTCTGAGTAGCTGTGGCTACAGGCTGCTCGTGGTTTATATAAAGCTGCATTCTTCTGTACCATGGAAATTATTCTATA
AAGTCTTTCCATATATAATGCCAATTCATGCCAGATGCGTACATCACTGTAATCTTTTTTAAAGATTTTTAACTGGCATTGTCGTTTAT
CCTATAATCGGGTAACCATTCATCCCATAAATAGAACTATTCTTTCCATTACCTATAACCTCATGACCCCTGTGCAAGGCCAAGTGCTA
GGCTTGGCACCTTCTGGACAGAGGTGAGGTTCAGAAACTTGACTTTCAGAAGACACTGTAGCAGGATTGGGTTTTGCTCCAGGTTTGCC
CAGGAGTCTTACTGTCACTCTGTCACATAACGAGGTTCCTACAGCAACCACAGTCAGTCTAGTGATGGTGCCCTAGAGCCCTCCTGGCC
ACACAGAATGGAAATGAGCTCTGCTCGGCTCCCTTTCCCTTGCCACTCAGGATTAGGAAAAGAAATAACTGCTTTCGAGAACTTTCCAA
CTGGCTATCAGCGCACAGAACTTTAATCTCATTTTTCTTCTTCACAGATAACAAAATGAGATCCATGCCTTGTTTCTGTTTGGTTTTGT
TTGAGAGAGAGAGAGAGAGAGAGAATATCACTGTGTAACCTTGACCTTCTGGGACATTGTGGGTGGAGCTCACTACAGCTGGCTACAGA
ATGAGTTTTGGGAAGGCAGGCTTTTAAATTGTTTCTGAATATTTAAAGTATTTCCGAAACATCCCAGGTTTTTTTGGTACACACCTTTA
ATCTCCACAACATATTATTTGTGTTATAAACATTTATAGTTTTCCATTTCCTCCACATAAGCCTTCTTCCTATTTTCCTTTTTTCTCCC
ATGTCCAGAACATATTATTTGTGTTATAAACATTTATAGTTTTCCATTTCCTGCAGATAAGCCTTCTTCCTATTTTCCTTTTTTCTGCC
AGATTGTGCCAAGAGTCATGACGAGGACAGTCTGACTCTCCCTCACAAGGAAACTGAACGTGAAGGGCCCACCCTGCCCATGCCCTGCA
CCAAGACGGAACCCATCGCCTTGGCATCCACCATGGAAGACAAGGACAGCAGGATGGGATTTCAGCCTAGGTGACCTTGCCCACGAGGG
CTTCCAGGGACAGCTGGGGTGGGTTTCACGCTACGTGACCAAAGCCCACAAGGGCTTCCAGGCACACCTGGGTACGGGCAATTCTAGGT
ACAGGAAGCTGTCTCCAGAAGACAGGTTTCCTTCCTGTTAATCTAAAGCTGATGAGTCAGCATCTCCTCTGAGTGCTCTGCACCGTGCC
CTGTGCCCTGCAACACAAACGCCTCCTGTGGATCCAGTCATCTTTGCAGCCTCAGCTCTGATTATAAGTGCAGAACTTGCATGTACATT
CCCTAGTCTGTGCAGTTCTGGGTATGAAAAGAGCCAACTCACTTTGTCCCTGGCCCAGGTAACAAGTCTCCATGTCCTTCCCGCACCTC
CCTCCCACCACCCGGCTCAGCTCCCACACTGAAGACGTTTGCCTAAAGAATCGCACTTTTCCTTTATTCTCTTGTCCTCCTTCAAAAGT
AGAATTGAAAGGGAACGTTCCATCTCACCAGGCCTCATTCGCATATGAGTCATGATTCTCGATCAGACGCCATCTGTAAAGAGATAGTA
ACTATTTACACATACTGGCCGTACACATGGTTACTATCACATATTTTTTTCCCTTTTTCTGTTTTTAAAAATACTTCTTCTGAGATGCA
CACTATATGTGTACAGAGCCATATGGACAGGGTAGATTTGCCCATTTGGGTTGAGCTTAGTCTGCTGTCCCTTGACCTTAAAAGATAAT
AAAATTTCCAAAACCCCTGGGGTTTCTGAGACTCCCTGCCACCCAAGGGTGTGTTTCCTCACTTTGACTCACACAGGATTCCAGAACAG
ACTTTCTTTGTAAAGTGATTCGCTATTTAAAATTATGGCAAAAGCCAAGTACCCTGAGATTGGTTGCTCCCCTTCAATCATAGACACTG
ATTGTTCTTCTCAAAATAGCAGATTTTCTAGATTGTTCCTCACAGACACTTTAAATCAACCTCTTAACTAATATTAATCTGTCAGTCAG
ACAATGCCCGAAATCCCCGATTGTGCTTGTTCTAGTCAAACACAAATCCAGAGGCAGCAAAAGGAGCATTTTCAGCTAATGCTGTGCCA
TGCCGCACACGCGGGGCGAGCAAATGACACTCAGCTGTGCTTGTGGGCATTACCTTCCAGATAACCTCTTTCTCGAGAAGGCTCTGCAG
CTCGCCAGGCGACACGCCAACGCCCTTTTCGACTACGCACTCACGGGGGATGTGAAGATGTTGCTGGCCGTGCTACGCCATCTCACCGC
CGTGCAGGATGAGAATCGGCACAGGTGAGTTGAGTGGGGACTGTTGCACAGCAAGCTCTGTTTCAGGACAAGGAAAGGCAAGGATACTC
CCCCCACAACTGCCTTTGAAGGCAAAGCTGCCCTCATCTGACAAGACCTCGAGTATGCTGTCTCCCTAGGCAAGAAGGCTCAGATGTGT
GTAATCATGCTAATTTTACACCCCTGCTTTGTGTACTAAAATAGTAAATTGAGAAAATTATTGACAGCCCTGCAAGTCCCTGAAGTGTT
ATTGTCATAGTGTACCCAAGAAACCCAGGTAAGAACAACATTAGTAGAGTAACTAAGAGGAAATGCCATTCTCTCCTAGCTACTAGCTG
CTTCTCTGTGGCCTCTGTTAAGGGTCACGAGGATGTGAGTGGGTCAGGCTCTGCCTCAGCTCCAGGCCAGGCTGTGTGGGAGTCTCACA
TTCCAGATGCCATGCCTGCTCTCTCTTCCTGAGGTTCTGTGCACATAGCTACCTGGCTCCTTCCTAAATGGTTCTCTTAGTCCTTCCAT
TGAGACTTCTGAAATGATCTAAAAGCCTATAAATATAGCCAGAGACAGCAGTCTTGAGATTCAAGTGCTTTATCTAAGTGATCCTAAAA
TAGCGTTAACACTGAAAGAAGCCCTTTGCTGTGGAAGCATTCTGGAGTGCTGTCTGCTGAGTCTCAGTTTAAAAAAACAAGCGCTTTAG
GAGAATGCCAACCTCAGGGGTTTCGTACAAGGACCTTTGTCCTCGTCACTTGCAGTGTAACTAAGGATGTGGTATAACCGCTGGTCTTA
TCCTACACAGTACAAGAGGAACCCAGACGACCTTCTGTGAAAAAAGCTCTGAATAACAGAAGGCGCCTTTAGAAGGAAATCATCCAGTT
AGCATTCAATAGCAAATAACCACAAACAGAGCAGATAACCAGAGCAGGACACCGGTGCTCCAAAAACGGTCATCTTTAGGACTGGCGAA
TTCATGAGTAGTGAGGGCGAGTGGATTAAAGCAATTAGCAAGTGAGATGCTGCTGTCTGAAGGAGAGGCTGTGGAAGGGATCCCAGGCA
CATGCAGTCCACCAAGTGTGTGCAGAGGAATGCAGGAACATGTGTGTAAGGACCTGGGTGTTCATGGGGCAGCTGAGACACTGGCCTTC
ATTCCCAAGTGGGTACAATACCCAGACCCTCGTCAAGCCAAGACTTAAACAAGCAGGCTCCGTGTAAGGTTAGGGGTATCAGACCTGGG
AGACACAGAGGACCCACTCAGGAGTATACATAAGGGACTATTCTGGTTTCTCTTCTTTATTCATCTTGTCATATTTGGCTTATAATCTG
GACCCTGTCACCATTAGTCATCATAGAAATACAATAAAGAGAAACTATAGAACCCTCTGCTATCGGGGAACTTCCCCCAGGGGTATGAA
GATCTGAAGGGGAGGAAGACGTGCTTCTGTGAAGTGCTGAGCTGCACATGGAATGTTGAAGTAGCTTTTCAAGCCCTTTTACTCGACGT
GTGTCTGTCCGCATCACCTCTCACAAGCCTCCTGTCAGGGTCCCATCAGGACCACCACAGGAGCCTGAGGTGTGGCTGCGGTGCTTCTC
GTCAAAGCGTGCGTGCTTCACTTGCAGTTAACACAGGCCTGGGGTCATCTAACAACGCCACCATGATCTTCCCTGAGAAGTATTGCTAA
GTGAAATATCTCAGGGATATAAACAAGACCTTCTTTAGAAAATAGAAGTCACTTCTCATTGTTACGGAGAATAAGACCTCGGCAGCTGA
GTTTTCCCAAGCTGCTCCTAAGCCTAGGCATTACCTTTCTTCATGGTCTTGTATGTCAGGGACAGACCAGGGAGAGCTGCCAGGGACAG
AGGCATGGAGACAGAGCTGGCTTCGAGCTTAATGCCATTTGGTTTCCGACCCGGCAGGTGCCTTTGCACTTCATGTGTCTGGTCAGATG
TTAACACTCCGGGGGCAGGTGGGCACGATCAGTGTGTAGTAGCCTGCGATAGGCAGTGGAGAGGAAGTTCTCCAGAGAAATAATGTCAC
CTCTGATCAGTACATTCAGTACATGGTAGGGCCAAGGAAAGCTACACATAGTAGCTAGTCTTCAGCTCCAGTCATTTCTTGACATGTTT
TTGTTTCTGTCGCTTGCTACATCCCCAGTTAATTATTGCCACTGTGTGTCATTCCAGTGTCTTACACTTAGCCATCATCCACCTCCACG
CTCACCTTGTGAGGGATCTGCTGGAAAGTCACATCTGGTTTGATCTCTGATGACATCATCAACATGAGAATGACCTGTATCAGGTAAGC
AGCCACCACAAGGCCGTCCACAAGAGAAAGCTTGCCTTAGTAAACACGTGCGTGAGCTGTTCTCACTGATGCGAACAGGAAAAACAGCA
TCTTCTCAGAAGTGGCATCTTAAAAGCATCCTTAGTTGGCAATGTCATTTTACAGAGAGATGGGTTGGGATCATGGGTTTCATGCCAAT
CACACTCCATCCAAATTCACCATCTTCTAGAAAAAAGGCAGACTTTAAGACCTAGAAGTCACAGTTGATAAGTTTTGAGGGCAACCTCA
GAGGGGAAAAAGTCACAGCTGTCACGAGAGACATGTTAAAAAGAAAATCCAAGTTTCAAAAGCCAGAGTTACACAGACAAGTTTGGAGC
TGAGATCAAAGGATGGACCATCCAGAGACTGTCCCACCCGGGGATCCATCCCATAATCAGCCACAAAACGCAGACACTATTGCATATGC
CTGCAAGATTTTGCGGAAAGGACCCTGATATAGCTGTTTCTTGTGAGGCTATGCTGGGGCCTAGCAAACACACAAGTGGATGCTCACAT
TCAGCTATTGGATGGATCACAGGGCTCCAAATGGAGGACCTAGAGAAAACACCCAAGGAGCTGAAGGGGTCTGCAACCCTATAGGTGGA
ACAACAATATQAACTAACCAATACCCCCAGAGCTCATGTCTCTAGCTGCATATGTAGCAGAAAATGGCATAGTCGACCATCACTGGAAA
GAGAGGCCCCTTGGTCTTACACTTTATATGCCCCAGTACAGGGGAACGCCAGGGCCAAAGAAGCAQGAGTGGGTAGGTAGGGGAAAAAG
GGCGGGAGGAGGGTATAGGGGACTTTCGGGATAGCATTTGAAATGCAAATGAAGAAAATACCTAATAAAAAATTGGAAAAAAAGAATAT
TTTCAAATTAAAAAAAAAAAAAAAGAATTTTGAGGTCTGCGGAAGGAAGGGTATTCGCACAGCAACTGGATCAAGTCTATGTCAAGAAG
GCTCTGAGGTCAGCTCCTGGAGAGGTGAAAGGCTGTAAGAACAGGAAGAGGCACCTCAGAGTCCCATCCAGCTCACCTGTGAGAAGGTA
AGGGAGCCCCGAAAACTCCAGAGGAACCACCTGGAGATGGCACAGTGACCTCAGGACAGAGGCAGATGGTAGGGGAAGCAAAAACCAGA
AACTCCACTGGCAGACACACTCTGGGAGCTGTGGCCTGGCCCAGGATGGTGACCTGCTCTACGGCTCCTTCCTCTCTCACCTCTATTTC
TCTCCAACCCTTTTGTGCCCCTACCCCTCTCTCTGGCCCTGTTGGTCTTATCCTCTTGTTGGCTTCTAAGTTCCTCTGCACTCTCTTGC
CCAGAGTGTCATTCTGATCCCCACAGCCAGGCCCATGCTGACACCTCTGCTTCCAGGTCATATTGCTTTCATTGAACTGTAAAACCAAA
CAACCGTGAATAATTTGTTATTTAGAAGACATGAGATGAGAACTAGTGGAATTCTTACTTGTTTGTTTGTTTGTTTGTGATGTTTTTTG
TTGTTGTTGTTGGTTTTTTTGGTTTGGTTTGGTGCTTGGGACTGTACCCAAAAGCTGAGCACCCCTTCCACCAAAATCTACAACCATGT
TACTCCATTTTTAAAACAAGTTATTCAAGTTGTTATCTCTTCTTATCCATGCTTCTCTCTGTCTCCCTCTGTGTCTCTCTCTGTCTCTC
TCTTCTCTGTCTGTCTGTCTCTGTGTCTGTGTGTATCCCTCTCTATCAAAATCTCTCTCTCTCTCTCTGTCTCTCTTTCTCTCCCTGTG
TCTGTGTATCTCTCTCTATCAATCTCTCTCTGTCTCTCTGTCTCTCTCTTTCTCTCCCTGTGTCTGTGTATCTCTCTCTATCAATCTCT
CTCTCTCATTCTGTCTCCGTCTCTCCCCCCCCCCCAATGTGTGTGTATCTCTCTCTGTTGATCTCTCTCTAGCGTACCCAGGCTAACAC
TGAGCCATCAGTGTCCTACCTCAGCCTCCCATCATAGGCTTGACAAGCATGTGCCTGGCATGTTATCTCATTTTTAAAAATTTAAAAAA
AATCATTATTTTAGAAGAAATTAAAAAGAAACAAAGAGATGACTCAGTGGACGAAGTATCTGCTGTACAAGTGTGAAGACCATGTTCAG
ATCCCCAGACCATCAACCAGCCAGATTTAGTACCACACCCCCCCACACCCCGCCCCCCCCCCCCCCTCCCCAGTCACGGCAGCAGAACG
CATCTTGCATATGCGGGGGAACGGGCGCGGACAAAAGACCCTGCCTTATAACCTTCCACAGGCAGGGGGCAGTACACACGTTCCCCTCT
ATACAATATTTTAAAGTTACTTTGAAAATATGAATTGGAGGCTCAACAAGTGGCTCACATCCTTACCCTTGAGTTCGTTCCCTCCCCAC
ACCAAACAAAAGCTGAAAACTCTTACTTCCTTGGCCTCTACCTCTCAGACTTTTCCGTTTTCAGAGGAACACAGCTGCTTTCTGCACAG
CACTCTCCACTTCTCTGTCTGCAGTGATTCCTATCCACCGGAGATGACTAGGCTACGTGTTATTTTAGATAATGTTCTTCAAGATCTGT
CTTCCTCGCCCTTCCATGCCATCTTCGGATACTATTGGTATTTGGAAAGTCATTGGGCCCCGATTGAAAATTCTCCAAAACTTTTAGGC
AGATGGCACTTTTTAAAAACTTTGTATTGATTCTCTGTGAGTTTTGCATCCTTTTTTTTTTTTTTTTTTTTTTTTTTTTTGGTTTTTCG
AGACAGGGTTTCTCTGTGTAGCCCTGGCTGTCCTGGCACTCACTTTGTACACTACGCATCATCCACCCCAGTCCCACTCATCTCCCTGT
CCCCTCATATCTGCCCTCTGCCCTTGCAGCCTCCCTCCCACAAAATAAACACTCAAGTAACAGGAAAGACAAAAACAAAAAAAGCATAG
AGAACATCTCGATGTGGGAGCGATAGTGTGTCACCGGCCATCCCACAGTGTATCCCTCTGTCCACACATCTTCACTTCCAATGAGTCTT
TGGTTCTCTGTGACACGGTAATTCTGAGTTCCCCAAAGTATAAGCTTTTATGTTATTTCAAGATTATGCTTATACTGAATTTACAATAA
TAACAAACATGTGGCAAAAGTGACAGCCTGAGAACTAAAACGACATACCCCGTGTCACATGCCGGAGAAAATCCGGAACAGAACGCACT
TTAAACACTCACACTTGGGCTAGTCTAGAGAGTGGCTGGGTTCCAACACCAAGATCTGATAGTGACAGATGGTCACAGATGTACTATGG
TTAGTGGAACATCTTTGCCGACAAATGTTTGAGATAATTTACAATAATCCAATACTTAGAGAACTCTATACTTTTCTGCCAAGAAAATA
GTTATTATGTCATGCATGATGGTGCATATCTATAATTCCAGCCCAGAGGAGTCTGAGGCAGGAGGATTGTTGTGAGTTTTGGCCCATTC
TGGCCTATACAGTGGGTTCCAATTTTGTCTGTATTATATTATAGGGTCTTGTTTAAACAACAAGAACAACAACAACACAAAACCAAACA
AGCAAAGAAAAAAAACCCTCCAAATTAAAAAGAAACTAGTTCCCAAGCTGGGTGTGGGGCACACATTTGTAGTTCTAACACCCAAGAAG
ACACAGGAGGACTGACAGCTTCATGTCAATCTGGGCTACACAGCAAGTTCTCGCCCAGCCTCAAACACAGGAAGACCCTCTCTCAAACA
AAACAAAGCAAAACAACTCCACAAAGCCAAACAAAGAAACCCAAAAGTTATGAGAATCACAAACGCTGCCTCTCTATGTCCTTGAGGCT
AAGAAGCTGGTATGGCAGGGAATGTTTGTGCCCATTAACTGAGTCAAGCCAAAGGTGGGAAAAACTGGGGAGAAGGTCTGAGGGCTCCA
AATCAGAGCAGCTTTGCCTTGAGCTCAGGGGAGAGGTTCCATCCACAAAGAGCTGGGTCGCACACAATAGAGCCTGCTTCCGCACATCC
TATCACCAACAGCCAGCAGGGCTTCCTGAATCCAACCTCCAGCAGCCGAGGGAAGCAGGTTTTGCAAGAGGATTGGGAAAGTAGCAACT
TCCTCTTCTTCAGTTTATTCTTATACTTGTGATGGTTAAAAATGCATTCTGCCACTTCTCTGCTATGAGGTAGCCTGGGTGCAGGGGTG
ATGCCTTCCTCCACCTCGCCTCTCACTACCTGTACACGCAGTCAGGAGAGCTGGCCTGGGGTCATGCAAGGGGGTGACCTACCCCTGTC
CTTCACAGGCTTTCGAGAGCAGGCCTTGCATCTAGCCTGTGCTGCAGAGTAGAGAGGACCCTCGTGGAGGGAGCACAGTGAGCAGCCCT
GAGGGTGTGAGAGCAGCACAACTGGCCCTGCCCCTCACTCACAGGCTGTAGTGTATGGGAGAGTGGCTCCTGCCCTTTGGGCAGCACAG
TGGAGCCAGCTCTGGAGGCATAGGGAGATCCGACAGCTCCCTATTTCCAGATGTGACTGTTGCATGTATACAGGAAGAGTGCTCTTCCA
GTGTTGGCTACAGACACCTGCTCTGGGAAGACATGTATTCTTTAGTGTGCTCACATGCACTCCACACGGTTTTATACATACATTTCCTA
TCCCACATCAGCCTTTCCCTTTAAGTTTCAATTTAAAATACTTTCTCAATTCATAATTTTTGCAAAATTCAACTGAGAGGACCTGTATA
TTTCCTGAGATTCAACCAGACGCTTACCTCCTAAGTTCCCCCAGCACTTACATCCTACTTAACACAATAATACAATTAGGAATTGCAAC
AGATTCTAGTCAGATCTCAATATTTGTATGTACTCATATGCATGGATAGCTCTGTAAGATTTTTTTTTCCTAAGCTGAACATGGTGGTA
TTGTGTTTTAGTTCCTGTTATTCAGGTAGCTAACGCAGGAAGAGTCCTTGAATATAGATGTTCCGATGTAGCCTGGAACAGAAGCAACA
CTGTTTCTAAGCAGAGTGAATGAATGAATGAATGAATGGTCAATCCATGTGCTTGTCCATGTGTATGTAGCATTCTACTGTGTCCCTGT
CTTGGGTCTTCACGCTGTTTATCCTTTAGTTTCATAGACAGCTGGTTTGTTCTCATTGCTCCTGAAGAGAATGCTGTCATGTCCCCTAA
CTAGCTGCTCACTGTCCACAGGCTGTGCACTGCAGTGAGCACCTCCACTGTACCCATGGGTGCCACAAGTGGGTTTCAAGAACAGTGTG
AGCCCTGAGCTCCACAGATGGATTCTAGGGATTCCTTGAGGCACGCATTTTATGCCTGTGTAAAATGTAACCCTTGGCTGTTGGTGGCC
CACCAGCCACTGCTTCCCAGCAGGAAACTCCCTGAATGCGAGGTCGGGCAAGTCCTCACTACACACTAACCTGGGCTTCAGTCTAAGCG
CCTACAACACAGACTGGCTGGCATTGACAGTTGGTGCTACTCTCTACCAGTGATCTCAGCTTTTTGACAGACACTTTTGTGATTGGGTT
AGCCCCACTTGTTTCTCAAAACAGTTTTCCAATGCGTCCTACTTACAACCATCGAAGCACAGCTTTCAGGTCAGGGTAGTCAGAAGACC
TCCCAGAGCCTATTATGCACAGTGCCTGGGATTGTTTTCTGCCTGAACTTTCTTAGGGAGAGTGCTGTATTGGCTAACTATCCCCTGAG
GATCTGGTAGCACCTGTGATAGTATTACAGTGGAGCTTGTATCTGTGCGTCTCTTGAAGCCAGCCTGCAGCAGTGTGTTCTGGTTTAGC
CTATGTACTTCCAGACCATTGTGGAAGGATGACATCTTTCTTGATTGCTTTCTTTTAATAGTCTATTTATGTTTTGTTGTTAAAAACAC
AGCATTGGTTCGATTTGCAAATGTATAGAATTACCAGCTATAGATAGATACCTATCAGAAATTCAGAGAAATTAAATTTTCTTCTACAA
GAGTTAAAAAAATCATGTAGCAAAACGTATCCCATAACCCCGACTGTTGAATGCTGGTTCCCTCCGTGGGCCTTTGAGCCTGGCATGCA
GAACAGGAAGCTCCTTGAGTGACAATGTCCCTACAGTATGACTCCATAACGTAGCCTGAAGCCAGGGGCTACAGACATCCCTAAAACAA
TCGCTCTCCCTCTCCTTCTCTGTGTAGACACCTCTGCACTTGCCCGTGATCACCAAGCAGGAAGATGTAGTAGAGGATTTGCTGAGGGT
TGGGGCTGACCTGAGCCTTCTCGACCGCTGGGGCAACTCTGTCCTGCACCTAGCTGCCAAAGAAGGACACGACAGAATCCTCAGCATCC
TGCTCAAGAGCAGAAAGCAGCGCCCCTTATCGACCACCCCAAGGGGGGAAGGTAAGAGCAGTCGCCTTGCTGGCTAACCGTCCCCTCTC
GGCTGCAGCATAAGGGCTTGGCATTTGACAAATGCGTCTGACATGACTTTCACCATCAATACTCTCCTCGGCCAGCTGGAGTTTGTCAT
ATCTTGTGGTTATTTCTGCTGTTTATTACAGCAAACGCCCTTAAAGAGTATTTGTGAGTATTGCTTAGTAAGTGGAAAAGCAAGCCCCA
GCAAACATTAGTCTTTATAAAGCATCGTGTGTGTGTGTGTGTGTCTGTGTGTGTGTGTGTGTGTGTGTGGAGAACGTTTACATAGTAGC
TCAGTGTCTCCCGAGAACACAGTGGGAGCTTGTGGCTTCTGAAAAGTAGAACAGTGGACCGTCTGAGGCTTTGAACACGCCCTGAACAG
AAACCGGAAGTGCTGCGACCTGGTGGTGTCCCTCCTCCCCGCAGCCCAGCTCTGTCAGTCTCACCCACATCTCTGCCTTTGTTCGGCCT
TAGGTCTAATCCCATCCACATAGCTGTGATGAGCAATAGCCTGCCATGTCTGCTGCTGCTGGTGCCTGCCGGGGCACAAAGTCAATGCT
CAGGAGCAGAAGTCTGGGCGCACAGCCCTCCACCTGGCCGTGGAGTACGACAACATCTCCTTGGCTGGCTGCCTGCTTCTGCAGGTAAA
GGCGTACTTGTGATCTTGACCTAAATCCCCCTGGAAGTTTTAGGGAAGTCTTTTCAAAGAATGACTCTAGGGCCAGCTCGCTGTCAGTA
TTCCACTGTCTGCTTTCTGCATCCCCAGCTCCAGCCCAGCTCTCAACCTGGGGATGCCAGTGACACAGCCAGCCCAGTATAGCAGTTGG
GTGAATGATGTAACCTGAGGTGCAACGGAGATGTCGAGCTAGACAGTGGAGGAGAGAGCCCCTGCTAACTCTTTCTTTGTGGGCGCATT
TAGCGTGATGCCCACGTGGACAGTACCACCTATGATGGGACTACACCTCTGCATATAGCGGCCGGAAGAGGGTCCACCAGACTGGCACC
TCTTCTCAAAGCACCACGTAAGACGCTGTGGTCACTGATACCAGCAAAGCAACTGCCTGCAGTTCAAAAAACTGCCTGCGGAAGCTGCG
GTTCTGCCCTGGGAAAGTGCCGGTAAACCACACCTGCACCCGGGCCACTCTGCAGCGGCTGCACTGGAGGCTCTGCTTGGCCTCGGGTT
TACCTTGATCCTCACGCTAGTTCCCACTCTCTGGTTATCTACGTTGCCTTTCGGTTGTTGAGGTTTACTCGGTACCTATAAAATCTTGA
GCTGTGTCACCTTCTCGTCATATTACAAGTCTCCCAGAAGGATATCTAAAGACTTCCTTTTGCAAAAAAAAAAAAAAAAAAAAAAGTCT
CTTAGAAGCTGAAAGCATGGGAAAAGCAAATGAGCCCTGTATTTGTGTTACTGTGCACGAGGCACTAAGACTTGCTGGCCTCCACTGCC
TTTCCTCGGCTCCAGGCACCATGCTGGCATCCCATATTCACAGTCTCCTTTACCACATCTACTCCTCAGTAACTCTCTGAGATAAGAAC
TGTTGCTATTAATCCCATCTGAGCACCAAGAAGGCAGACTACAAAGATGATC1AAGCCCCACTCCCACTCTCCCGGAAGCTGAGTCACA
GCACCGCAGCCTGACTGCAGACTCTTATTCTTCACGCTGTCTTTCTTTCTGCTCTGCCTGGGGTTCTAGTCCTTCCTGTAAACCACTCA
GAAACATTCCAGCTCATCTCCTGTCTCCGGCGCTTTGTGTGTTCAGTGTTAACACATGCTTGATGAGGTTGGCTGGCCTTCCGGGCCTG
CCCAGACACACCTGATTTACTTCACAGGTTGATAAGGGAGGATTTGTGAACGACACATTCCTTATAGATTCACCCTAACAGCCCTTGGT
TCTAATGTGTTGAGTCATTTTGGAAAGTCACTGATTATACTGGGGGAAAAAAAAAGGTTGTCTCTTACTCAGACACCTGAAATCTCAAC
ATTCAGAAGGCTGACGCACGAGAATGCCCACAAGCTTAGGCTACCTTGGTCCACATAGTTCTAGCCAGAACAAACATACATAGTGAGAT
ACTCTCTCGACAGTAAATATATAAATGTAAACACTTTTACTTGAATTCACCATGAAAAGGTAGCAAATAATATCTTGTGTGACATAAAG
AAAACCAGACAAAAAGCCTAGCCCTGACCTCCTCGCAGTCACAAAGGTCACTTCAGCCACATCTCTACCCCTCGCGTCTTCAAACTCAT
TTCCCAGTCATGTTCCTGGCCATGTTTTGCTCACGTTATAAAACAGCAAGTGAGGCACCACAGACTCGCAACGCAGCACTCTTCCCCCA
ACTCAGAGGTTCTCTCGGCAGAACTGAAGCAAACACTTCATTCCAAAACGCTGTGCTGAGCCTGCCTGCCTGCCTGCCACTGTTGGCCA
ACCCGGAGCCGGACACCCATAGGTAGAATACCCAGGAAGCCATCACAGGTAACTCCACTAACATTAGGGTTACGTTTTATGTTCTCTTG
CTGGGAGCTGAACAACAGCCAATATCCCCAAGATTTGTTACCACATTTTTTCCCCATAACAACACATCCTGCCTAGCATAGCGGCCTTG
TGGTGTCCATTTCTCTGTTCCTTACGAACTAGAACTAGGGCCCCAACAGTACAGCACTCAAGGACACAGCTGACCAACCAAATCTGCTC
CACACAAGTAAGCGAGAGTGTGACAAGTGTGTATTCGTACATCTAAATGTGAAGGTTCAGGACTTTACCCCTGTGGGTGCATAAATTCC
CACCTACTAGTCCTTACCTTAAATGTATCCTTTGTACSTATAATAATAATAAGAATAATAATCCTGCAGAGTTACAAATCCCAGGAGTA
CTTGAAGTCTTTCATGTATTTACTTTACATCCTGATCACGTCCTCCCCTCCTCCCAGTGCCCTCCACATGGCTCCTCCTCCCCACCCCC
TCCCTCTCTCTTCACAGGGTCCTTGGCTACCTACTTCCATAGAAGTGAATTAAAATTTTTTAAAAAGTTGTTTTAAATTTCAAATAAAA
TCAAAAGTTATCCTGTAATTGCCTTAATGACCTACACGTAACCTGTCCCTCGACATGGGAATCCTGTGTGTCATTGGAGGCGCACAAGC
TTTTTCTTTTACACAACACTGTATTTCCCTGACTCCATAGGAGCAGACCCCCTGCTGCACAACTTTGAGCCTCTCTATGACCTGCACCA
CTCTTCCGAGAAGGCTGGAGAACATCACGGAGTGGTGCCAGGTACCACACCCCTGGACATCGCTGCCAACTGGCAGGTCAGCTCTGGCC
AGCCCATCTCATGGGTATTTACGGCCCAGCCAGTGCCTCAGGAATATACAGATGGTCTTCTTGGCATGCTCCTCCCTAAAAAAGAACAT
GCCTTTGTTTTTATCAGGTCTTGTTACCGAGAGGGTCTTAATCTTGAAAGATGGTCTTCATCCATGAAAGATGAAGGTCCGGTGAGCCA
GAGTGTAAACCTGTTTGCGTAACTGGATTAGAGTTCAGATATGAGAGGTTAGAAAAGCACAAAATGCACAGAACAGTTACCCTGTCACC
ACACCACCAGAGTTCTTGCCATTTCTGTCAGAAGCTAGCTTGCTCAGAGCTTAAGTAAGACGTTTCTGAACATAAAAATAAACTAGAAA
TTCTCTTGTGGATTTCCATAAACATATTAAAAACCTTCCTTAAAACACAATAGGGCTTACACTTAAACCACAAAACGCTAGGTCTTTGG
ACCAAGAGAAAAGAGCCAGAAGGCAAAGTAGGCAGTGTACGCCTCCAACACAGTCGCGTTTGTAGGAATCTAGACTCTCTGTGCACGTC
TACGAGGCTGTTTGCTCGCTGGGTTCCTACAGGGAACACAGAAGACTGACCCCTGACCTGATTCCTACCCTGTGTACCATCAGCAAATT
CACCTCAGAGAACACCAGGACTTTTAAAAATCATTTCTGTGAATATTTTCTCATGTTTTTCTATCCGACAGGTATTTGACATACTAAAT
CGGAAACCCTATGAGCCTGTGTTCACATCTGATGATATACTACCACAACGTCAGGTCTCGCAGGACAGATTCTCTCAGCCAGTGTTGCC
CCATGCTGTGGTTTACAGGAGGAGAGGAGACGCCAGCCTGGGCGAGGGTGTACTGCAAACCGTCTAGATCTTATCTCAGAAAATCAGAT
CCCTGGAGGTGTGGCCCTGAGCTGCTAGAAGCACCTACTGCTCCTGCTGAGCAGGGCCTCTGGACTTGGCAGAAGCTTGGTGGCCTCCA
TCACAAGTTGGACAATGACCTGTCCTCTCAGATTGATTGTCCCTGGAAGGTTAAGCAGTCATGTTTCTTCCTTAGGGGACATGAAGCAA
GCTGACAGAAGACACGAGGCTACAACTCTGCAAACTGCTGGAAAATTCCTGATCCAGACAAAACTGGGCCACTCTGGCACAGAAGTTGG
GTCTGGGGATATTGAACAATGCCTTCCGGCTGAGTCCTGCTCCTTCTAAAACTCTCATGCACAACTATGAGCTAACGCGCCGCCGTACC
ATTTAGAGTTGTCTGTGTAACTATTGGTAGTTGACCGTGTCAGCTCCACTGAGCAGCACAGCCTACCCCCTTACATAGTAACTTTATGG
AAGGAAACCAGTCATTGCCTGGAAGGCAAAGGGAGCCAGGCCAGGATGGTGCCTTTTTCTTGACAAGACTGGTTACCCGTCTCTCGGCA
TTCCCTGTTTTCAAATTCTCAGAGCCTTCCTGTGAAAATACTACTGCCCACAGGATGGGGACCCCTCCAAGCTGTGATCGGTGTGTGCA
TCCCTGTTTGCAAAGAGGGAGCCCCCCAGGGCCATGGCTTCATTGTTCTGTCGGGTGACTCCTATTAGAGCAATTCAACTAAAAAAATA
GACAAACATGGGCGAGCTACTCCGAATGAGGCTGTCATTGCTAGCTGCTGTCTTCCCTGGATTTCTGTGCGTAGACATCACCGTAGGCA
GTCTCCTCTCTCCTGTCTCCATGAACTCACAGGCTTCTTTCCCTTCCTTCCCCTCTTTTTTCTTATCTCTCTCCCCNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNCTACACAGAGGCCATTGAAGTGATCCAGGC
AGCCTTCCGCACCCCGGCAACCACAGCCTCCAGCCCCGTGACCACTGCTCAGGTCCACTGTCTCCCTCTCTCCTCTTCCTCCACGAGGC
AGCACATAGGTAAAGAGGCTGAGAGAGTTCCTTTCCTCGCTGGCAAGGTCTTTGTGCTCTGGGACGTGGCACTACACTCCCTTTCCCTA
GTCTTTTTCCTGACATGTGTTCCTCTGTCGCATGCCACCCGCTCCCTCCCCACAGTGCTCCTCTCCGCACACAGGAACAGGCAGTTCCC
ACAGACACTCACATTGGCATATAGGTCACTTAGAATCCCAGTAGACACTGGACATGGCGGGGACCGTTAAAGAAACACTACTTCTCATA
TTTGTTAAATTTGGAGAAAGATTCTAAGAAACCTGCACAGCCCTGGCCCACAGGACAAGAAGTAGGACCCAGGAAAAATGCATACTGTA
TCACCCTTCCTTTCATGCCACCACCCTTCAGGTCCCCCACCTCCTCCCTCCCTCGGCACTCCCCGTCCTTCAGCTCCCACCCTGCTCAT
GGTCCTGCCTTTGTCTTCCCACACTAACATCCACCTGTCACCCCCAACACATGCATTTGTACGAATGGATCTCCGTACAGGCTTCAATT
ATGTCATTGTTCCCGGCCCGTGCTTTCTCTAACCTCTGCACCCTTTCTTCTTGTTCATTTTCTACCCTAGCCACAAACTCCACTGAGAC
CAGGAACAACTAGCATTCTCCCATCTTTGTATTATGCCAGCACCCCGGTGGCAGGCCTTCCATCTGCGCTCAGTAGGTTATGTCTGCCT
TCCACCTGCGGCAGTCCTCCCCTGGGACCCTCCCTGTCACCTTCAGCAGGTCCTGGACACCTGATTCTTGCCTCTTTGTCCTGTACTGT
CCTTCCTTCCCCACCTGCTGTGGAGAACCTCTTGCTAGGATGTAACTTCTCATCTTTAAACACTGCCCCGCCTGGCCACAGGCTGCTTA
GCCCCGCTTTGATTAATCTTACATCTCAATGAATGGCTTTCTCCATTGATAGCGCTTTGGTTAGCTCATGTTTTCACGTTTGTATGATA
CATCCTGTTATTTTTGTTCGCATTTCTCATATGGGGGAATCTACAGGTAAACACAGCCTCCAAGTTCAGTAGAAAAATGTCATCATTAA
GAGTATTATATCCAATAAACAGTAAACAGATTTTTTAAAAGAGAAAGAAATTTAGAGAAAAACGAGACATCACTTCCTTGTTACACCCT
TTAGTTGAGTTCAAACTTCTCAGAACAATATGATAACTGCTGGTCAGCTCACCACCTCTGTCTGCCATCTGCTTCTGTCCCCTCCTCTC
CTANNNNNNNNNNNNNNNNNNNNTCCCATTTTCACATGTGTTCCCTACCCTTTCCATCCCTTTAAAGCCTGTTTTCCGCCCCCTGGAGG
CCTTTGCTAGTTTTCTGGCCTCTGCTCACGCTCACTTCCACATGAATATACATACGTGACTGTTGGAAGCTAGGATCCACACGTGAGAG
AGAACGAATGGTATTTATGTCTCTGGGCTGCCTTGATTAGTATTCTCCATTAACCTCCAACTTTCATCTCTCTTTACGGCCGAAATAAC
TTTTATCATCTTCACACAACTCTTGGCTCTTGCACAAAACTCATACTGGCCATGACTGTCAAGTATAGCCACGCTGTAACAAAAATCTA
AGAGTGGCAATCAATACTTTCTGCCATTCACGTCCTCATGCTTAACCCTGAAAGGGCATCTCTTCTTGGCAGATGCCAAGTTCCGCCCA
TCCTCACTGCTTGCCTGAACTCCAACAGTGGCTCCTAGTCAGCCTCTTTGACTCTTTGCTTGCCCCTCCATCAGTTCTCTGCACCACAG
TGATGTGACATGATAGGGCTCCAGTCACAATTAAAAACTAAATACTTAGCAGCTAGGAACACGCCAAGTGTCCCCTCCTCTACCTTTGG
GAAAGCCAGGTAGGCTGACAAATATGTACAACCCGATACCCCACTCATAGAGGAAAGGACACCGAGAGAGGTGGATCCACGGGACTCAC
TCGCCAGTCTCTAAAAACAAGATGGAGAAGGAACAAGGAAGTCACACAATGTTGACCTCCCCACACCCCACACCTGCACATGAGCACAC
ATCCCACAAACACAAAGCAAATACTTAAATACTGTATTAAATTTCCATCATTCTGACCAACTTAGGAGAGATTTGGTTGGGCACACAGT
TTTTATAGGTCCCATGGTCCACTCCCTTCGGGGTATAGCAAAACCCTTCATCACGGAGGAAGCACATGGTAGAGGAATCCTACTCACTT
CAAGGTGTTTGGAAGTACACAGAGATGGGGAGGAGCCCCAGTACAGATCTCTTTCAAAGCCACGCCCTCCATGACCTAACTTCCTTCTG
TGAGGCCCCATAGTCAAAGAGTTCTATCACCTCCAGATAATGTCACAGGCTAGAAACCAAGCCTTTACTTAGCACACAGCCTTTGTGGG
ACATTTTAAGATCCGAACCATAATACACAGTAAGCCTTTCAAATGCTGCCCAGTCCCTGTTACACAACAGACAGTTGACTGGATCAGGT
AACACCAGGACACCGCTCTGGCATGGTCATTGAGTCTCAGTGTTCTGACGTTAAGATTCAATGATGAGAGTCAAACATTTGCTTAGTAA
GTGTTATCCTTCTGTTTCTTTAGTCACTCTGTACAGAAATAATCTGCTCTGGTTCCCAGGAATGCTTTTCAGCTGATAGAATTTAACTT
AACCTTTTGGTATAGACACATTTAAAAAAAAAAAAAAAAAGCCTCATTTTCTTCCACCAAAAGAAAATTTTCTAGGCAATGTGTATTCT
TTAAAACCTCATACAGCTCAGAGAAACGTGCCATTTTTAGTTCCTCCTTCTGTTATGACTTTATTTTGCCTTGTATTCAGCACTTTGCC
CAGCCGTTAAAAAAAAAAAAAAAAAAAAAAAACACAAAAGCTAAATCATAGACCAGTTAGAAAAATATCCCTCACTTTACAAAGGAAAA
ACATGTTTATCCTGAACCAAGAGAATTCACAACAGATTCTGTTCAACAGAAAGACTGATCTACCCTCTAGCACTCTCTAATGTCATTTG
ATACCCTGCCATTCACACAGCCAAGCAGCACCGCTTGGCATGATGGGGAGGTGGGATGCCGTGCTCCGCAGGAGCTACCAACCACAACC
CTCCATACCATAGTCCACACGGAGCCTCGCAGGGAGCCCACTCCTGTGGATCGTGCTCTACGGCACTCTCACAAACCTCTCTGGGTTTA
TGTCAGAAGGAATTCAGGGCACCAACCCAAACACTTTGAATCTGAAACTCCTGAGCACCCCGGCTTCCTGCCTCATGGGCTCATCCTCC
GCTAGCTTTCTTTTCCTTTTTTTGTTTTGTTTTTTTTTTTTTTTTTTTCCAGACAGGGTTTCTCTACATAGCCTGAGCTGTCCTGGAGC
TCACTTTGTAGACCAGGCTGGCCTCCAACTCAGAAATCCGCCTGCCTCTGCCTCCCGAGTGCTGCCATTATAGGCGTCCCCCACCATGC
CCGTCTTCCGCTAGCTTTCTTACACAGCCCTGGACTCCCTCTCAGGGAGTGGTGCTGTCTACAGAGGATAGGCCCTCCTCCCGATCAGT
TAACTGTCAGGACACGCCCACAGACCAGTCTCATCTGGCTGGCCCTCCCTCATCTCAACACTCCCTCCTCAAGTGATTCTAGACGTGTG
AAGTTGACAGTTGAACAAGTCACAGCCACAGCACGCAGAACAGCCCTTCCCATGGCCTAACTCAGCCCCTCATCAGTCTGTGCTTTCTC
CGCAGATGAACTCCGGGATAGTGACAGCGTCTGTGACAGTGCTGTGGACACATCCTTCCGCAAACTCAGCTTTACAGAGTCTCTTACTG
GAGACAGCCCACTGCTATCTCTGAACAAAATGCCCCACGCTTATGGGCAGGAAGGACCTATTGAAGGCAAAATTTAGCCTCCTGGCCGT
TCCCCCACACTGTGTAAACCAAAGCCCTGACAGTCCATTGCATCGTCCCAAAGGAGGAAGGCAAAGCGAATCCAAAGGTGCTGGAGAAT
CCCCCGCCTGCAGGGTCACTCGATTTCATTCAAGGCCTTCCGAATTTGGCGTCCTTTCTTGGTTCTGAAATGAAATGTAGTTGCCACGC
ACAGACGGTGTCTAGCAATCATGCCGCTCGCTCGCTCAGCTGCACTCTATGGCTCAGGTGCAGTGTCTTGAACTTTCTCTGCTGCTACT
GGATCACATTTGCTTTGTGTTGTTACTGCTGTCCCTCCGCTGGGTTCCTGCTGTCATTAAAAGGTGTCGCTGTCCCCACCCCGTGTCCT
TTCTAGCCATCTACTGTAAGTTGTGCATTCAAATTAAGATTAAGGAAAAACATATTTTTAAAATGAGTACCTTCATGCGCAATAAAAAA
AGACATTTCTTTTTTTAATGTGGTTTATCTGTGATTTAAAAATAAAAAACACATGAACTTACAATATTTAAACATGCTACAATTCAGTG
CTGAAAATAGTATTTTCCCCGTTTTATGCATTTTACTATTGTAAATATGTTTTCTAAATCAATACTTTAAAAGAAGAAATGTTGAAATT
TATAAATGCTATTTACTTTTTTATTTTACTTTTATAATAAAAGTACAAGCACATTGTTGACCTAATCTTGTCTGATCTCTGCAAATGCT
TTCTCAGAGGAGTCCCTGTGACTGTGGGAACAGCCCCACAGCCCGTTTGTCACATCCACATTCTCCTTTAGACACATTTCTTTGAAGCT
AGAAATGTTCAAACTGTGGAAATAAAAGTGTTCTAACCCAAGACTGAGGTTCTGACCCAGGCAACATCATCTGGACGCCTAGCATCAAC
ATTCCAATCTCTACACCGTTCCTCTCCAGCGCATGCTGTCTGTGTCTATCAAGGATAACAGACCCCAGCTTCAGCACTTCATCTTCTGG
ATACCATGTGGCCGTTGCGTTCATAGCTAACTACACAAAACCTGTACAAGATGTGGTCCTTCATCATGTATACAAGAGGGACTGATCAA
TACACACACACACACACACACACACACAGACACCACAGCAACTGGCTGTTAATAGTTGCTAGGGGAATGGGAGTCCTTTCTTCAAATGG
TATAGCACATACTCCACTAACTATTCCCTCCCCATGTTCATGCAGGCAGCCCTAAATTAACTCACTCATACACACAAGCACAGAAGCAG
GAAAGTAGGAGGCGGAAACGAGAACCCAGCGGGGTTTGTGGATATGATCTCAGTACATCCTATACATGTTTAAGATCATGAGAAAATAT
ACATTTTAATTAAAAACTAAATTCCAACATATGTAAAAGATTTTCCAGTATCCAGTTTAGTAAGTCTGCATGGCTGAAAAGTAGAAGAG
GGAAGATTAATCGCTTGAAGAGGTCTTAGTTTGGACTCTGGTGTGTGACCAAAGGTCATTTAGTGTGTAGTGTGATCAAAAAATCCAAA
ACCTTCTGGTGAAGTGGCTTGGCAGTTAAGAGCACTGATCTTCCAGAGGACCAGTCAGTAGGTTTAATTCCTAGAACCCCTCACAGCTC
ACAACCACCTGTAACTTCGGTCCCAGGGAATATGATGCCCTCTTCAGGCTTCTGTGGGCACCAGGCATATAAATGGTGCACAGATATAC
ATGCAGGCAAAACATCCATACACACAGAAAGAAAAAACCAAAAACCTAAGGCCTTGTCATGCCACACAAGTGATCTACTACTCATCCCA
CGGAAGGGCCTGGAAACCCACCAGCTATTACCTTTGGGAACTGCCTTCACTACAAAGAGCTCTCACCCCCATTTCCATGGTAGTCATAT
GAATGCCTTTCCCATTGCTGGGACCTTCAGATCCTGGGAGACAGGCTAAAGTGATCTGGGCTATCTCCCTCACACAGCTCAAGGTACCC
CAAATAGACATCTTGTATGTCAGTGTCCATATGACAAAATATTCCAAACTTACAATGAAGAAAATGGAGGTCCAGAGACCGCAACTCAG
TTGTGGCTAAGGTGCATCTGTGTTTATATCCAGATACTTAGCGCCTAGGCTTGTGAGTTAGAGAATGATTTTATTGGGACAAAAAAGAT
GATAATGACTGAATCTATTAGTAGGGATTTGGTATAAGATTAAGTAAAAATATCATTAATGAAGAAATGATTAATTATTTAATATATAA
TACAAGCTACTTAATAATACTTAAATTATTTAATAAATATTCTAAGTGCCTAATCATCAATTAATCGGAATCCCTTTCTACCAAAAATT
CCATATGTATGAAAATTTAAACCATACGAATTTTAGACATACCACCAATCTTAGACAAACACCCTTGACATCGAGAAGGCCCTTATCGA
GACTTCATTCTGTGATCTAAAAGCAGAGCAGATTCTACCTCTTAGAATGCATCACAATGAAAAACAGAACTGGTTAACAGCAAATTCTG
TTTTCCATTACAGTACCCTCTCTCCACCCTAACACACCCTTATTCTGCGTTGGAAACTTTAAGACAAAACTGTCTTGCTTTGGTGTTTC
CTAATACTTCCTGGGACACCCATGCCACCATGCCCCAGCCAGCACTTTCTATGGCTGTTACCTCTCCGTCTTAAAACATGGTGCTAAAG
CCCTTCCCACAGAGAGACACCATTCTTTCCTCGCTTTAGGCTCCTGAGTCCAGGCCACCTGTGGCAAGGCAGGAACGATGCTGAGTGAC
TTCCGAAACTAGGTCCGAATTTCTGCCAACCTTCCCGAAACTACCTTCTCCTGGAACTCAGTCACCCTGTTCAGAGGGTGCCAAGCAGC
CATGAAGAGAAGGCCAGTACAGGTGTTCAGCCAACAGCCTTAACCGAGGTCCTAGCCAAAGCTTGCCAAGCCGACGGCTGCCAGGTGAC
CCTGCCTAGCCTTTAGGCTGTCTCAGCAGATGCTCACCACAATAAACATGGGCTGTTTTGCTGTTCTCAAAGGACAGATTTCAAAGCAA
ATTAAATACTAGTGGGGGTTTTAAGCCCTGGGTGTAGAGTGGACACCACCATTCTCCAGGCATTTGAAAAGACAGCTTGCCTACCATAA
GTTCAGCAGAAAACAAATACTGACTGGAGACACGC
MOUSE SEQUENCE - mRNA

(SEQ ID NO: 26)

AGCGGCCGCCGCGGGCGCGCTCTAGCAGCGCAGGCCGCACCTCAGGGCCCCGCGCGCCCGGCCCGCCCCGCGCTTCTCCGCCCGCGCCG
CAGCCATCGCCCGCCGCTGAGCCGCCCGCCCGCCCGCCCGCGCCCCGACCCGGCTCGGCTCCCGCCGCTCCGCGCCGCTCCGCAGCGGA
GCCCGCAGGCGAGGAGAGGCCCCCCGCATCTCCAGGGTACCCTCAGAGGCCAGAAGAGGGTGTCAGAGCCCTTGTAACTGGAGTTTGAC
GGTCGTGAGCTGCGCATCTTCACCATGGCAGACGATGATCCCTACGGAACTCCCCAAATGTTTCATTTGAACACTGCTTTGACTCACTC
AATATTTAATGCACAATTATATTCACCAGAAATACCACTGTCAACAGATGGCCCATACCTTCAAATATTAGACCAACCAAAACAGAGGG
GATTTCGATTCCGCTATGTGTGTGAACGCCCATCACACGCACGCCTTCCGGGAGCCTCTAGTGAGAAGAACAAGAAATCCTACCCACAG
GTCAAAATTTGCAACTATGTGGGGCCTGCAAAGGTTATCGTTCAGTTGGTCACAAAATGSAAAAACATCCACCTGCACGCCCACAGCCT
GGTGGGCAAGCACTGTGACGACGGGGTATGCACCGTAACAGCAGGACCCAAGGACATGGTGGTTGGCTTTGCAAACCTCGGAATACTTC
ATGTGACTAAGAAAAAGGTATTTGAAACACTGGAAGCACGGATGACAGAGCCGTGTATTAGGGGCTATAATCCTCGACTTCTGGTGCAT
TCTCACCTTGCCTATCTACAAGCAGAAGGCGGAGGAGACCGGCAACTCACAGACAGAGACAACCAGATCATCCCCCAGCCAGCCGTGCA
GCAGACCAAGGAGATGGACCTGAGCGTGGTGCGCCTCATGTTCACAGCCTTCCTCCCTGACAGCACTCGCAGCTTCACTCCGAGACTGG
AGCCTGTGGTGTCAGACGCCATCTATGATAGCAAAGCCCCGAATGCATCCAACCTGAAAATCGTGAGAATGGACAGAACAGCAGGATGT
GTGACGGGAGGGGAGGAGATTTACCTTCTCTGTGACAAGGTTCAAAGAGATGACATCCAGATTCGGTTTTATGAAGAGGAAGAAAATGG
CSGAGTTTGGGAAGGATTTCGGGACTTTTCCCCCACGGATGTTCATAGACAGTTTGCCATTGTCTTCACGCCAAAAGTATAAAGGATAA
TCAACATTACAAAGCCAGCTTCCGTGTTTGTTCACCTTCGGAGGAAATCAGACCTGGAAACTAGTGAACCGAAACCCTTTCTCTACTAC
CCTGAAATCAAAGACAAACAGGAAGTGCAAAGGAAACGCCAGAAGCTTATGCCCAACTTCTCGCACAGCTTCCCCGGCCGCAGTGGAGC
GAGCCGGTGGTCCAGCCATGTTCGGTAGTCGCGCTGGCGGAGGGAGTACCGGAAGCCCTGGCCCAGGGTATGCCTACTCGAAAACTACG
CATTTCCTCCCTACGGTGGGATTACATTCCATCCCGGAGTCACGATCCAACCCAGGGGTCACCCATGGCACCATAACACCAAAAAATTT
AAAAATCCCCCTAAAGATTGTCCCAAGACTGATGACGAGGAGAGTCTGACTCTCCCTGAGAAAGGAACTGAACGTCAAGCGCCCACCCT
GCCCATGGCCTGCACCAAGACCGAAACCCATCGCCTTCGCATCCACCATCCAAGACAACCAGCAGGACATGGGATTTCAGGATACCTCT
TTCTCGAGAAGGCTCTGCAGCTCGCCAGGCGACACGCCAACGCCCTTTTCGACTACGCACTGACGGGGGATGTGAAGATGTTCCTGGCG
GTGCAACGCCATCTCACCGCCGTGCAGGATGAGAAATCCCCACAGTCTCTTACACTTAGCCATCATCCACCTCCACGCTCAGCTCAAGA
GGATCTCCTGGAAGTCACATCTCCTTTGATCTCTGATGACATCATCAACATGAGAAATCACCTCTATCAGACACCTCTGCACTTCCCCG
TGATCACCAAGCAGGAAGATGTATAGACAAATTTGCTGAGGGTTGGGGCTGACCTGACCCTTCTGCACCGCTGGGGCAACTCTCTCCTG
CACCTACCTGCCAAAGAAGGACACGACAGAATCCTCAGCATCCTGCTCAAGAGCAGAAAACCACCGCCCCTTATCGACCACCCCAATGG
GGAAGCTCTAAATCCCATCCACATAGCTGTGATGAGCAATAGCCTGCCATGTCTGCTGCTCCTGGTGGCTGCCCGCCCAGAAGTCAATG
CTCACGAGCAGAAGTCTGGGCGCACGCCGCTGCACCTCGCCGTCGACTACCACAACATCTCCTTGCCTGGCTGCCTGCTTCTGGACGGT
CATGCCCACGTGCACACTACCACCTATGATGGGACTACACCTCTGCATATAGCGGCCGCAAGAGGGTCCACCAGACTCCCAGCTCTTCT
CAAGCAGCAGGAGCACACCCCCTCCTGCACAACTTTGACCCTCTCTATGACCTGGACGACTCTTGGCAGAAGGCTGGAGAAAAATCACG
CAGTGGTCCCAGGTACCACACCCCTGGACATGGCTGCCAACTGCCACGTATTTCACATACTAAATCCCAAACCGTATCAGCCTGTGTTC
ACATCTGATCATATACTACCACAAGGGGACATGAAGCAGCTGACAGAAGACACGAGGCTACAACTCTGCAATCTGCTCGAAATTCCTGA
TCCAGACAAAAACTGGGCCACTCTCGCACACAAGTTGGGTCTGGGGATATTGAACAATGCCTTCCGGCTGAGTCCTGCTCCTTCTAAAA
CTCTCATGGACAACTATGAGCTCTCTGGGGGTACCATCAGAGCTGATGGAGGCCCTGCAACAGATGSCAACTACACAGAGCCCATTGAA
GTGATCCAGGCAGCCTTCCCCACCCCCGCAACCACAGCCTCCAGCCCCGTGACCACTGCTCAGGTCCACTGTCTGCCTCTCTCGTCTTC
CTCCACGAGGCAGCACATAGATGAACTCCGGGATAGTGACAGCGTCTGTGACACTCGTGTCGACACATCCTTCCGCTAACTCAGCTTTA
CAGAGTCTCTTACTGGAGACAGCCCACTGCTATCTCTGAACAAAATGCCCCACGGTTATGGGCAGGAAGGACCTATTCAAGGCAAAATT
TAGCCTGCTGGCCGTTCCCCCACACTGTGTAAACCAAACCCCTGACAGTCCATTCCATCGTCCCAAAGGAGGAAGGCAAAGCGAATCCA
AAGGTGCTGGAGAATCGCCGGCCTGCAGGGTCACTCGATTTCATTCAAGGCCTTCCGAATTTGGCGTCCTTCTTGGTTCTGAAATGAAA
TGTAGTTGCCACGCACAGACGGTGTCTAGCAATCATGGCGCTCGCTCGCTCAGCTGCACTCTATGGCTCAGGTGCAGTGTCTTGAGCTT
TCTCTGCTGCTACTGGATCACATTTGCTTTGTGTTGTTACTGCTGTCCCTCCGCTCGGTTCCTGCTGTCATTAAAAGGTGTCGCTGTCC
CCACCCGGTGTCCTTTCTAGCCATCTACTGTAAGTTGTGCATTCAAATTAAGATTAAGGAAAAACATATTTTTAAATGAGTACCTTGAT
GCGCAATAAAAAAAAAGACATTTCTTTTTTTAATGTGGTTTATCTGTGATTTAAAAATAAAAAACACATGAACTTATCAATATTTAAAA
CATGCTACAATCAGTGNTGAAAATAGTATTTTCCCCGTTTTATGCATTTTACATTTGTAAATATGTTTTCTAATCAATACTTTAAAAGA
AGAATGTTGAATTTATAAAATGCTATTTACTTTTTTATTTATAATAAAGTACAGCACATGTGACT
MOUSE SEQUENCE - CODING

(SEQ ID NO: 27)

ATGGCAGACGATGATCCCTACGGAACTGGGCAAATGTTTCATTTGAACACTGCTTTGACTCACTCAATATTTAATGCAGAATTATATTC
ACCAGAAATACCACTGTCAACAGATGGCCCATACCTTCAAATATTAGAGCAACCAAAACAGAGGGGATTTCGATTCCGCTATGTGTGTG
AAGGCCCATCACACGGAGGGCTTCCGGGAGCCTCTAGTGAGAAGAACAAGAAATCCTACCCACAGGTCAAAATTTGCAACTATGTGGGG
CCTGCAAAGGTTATCGTTCAGTTGGTCACAAATGGAAAAAACATCCACCTGCACGCCCACAGCCTGGTGGGCAAGCACTGTGAGGACGG
GGTATGCACCGTAACAGCACGACCCAAGGACATGGTGGTTGGCTTTGCAAACCTGGGAATACTTCATGTGACTAAGAAAAAGGTATTTG
AAACACTGGAAGCACGGATGACAGAGGCGTGTATTAGGGGCTATAATCCTGGACTTCTCGTGCATTCTGACCTTGCCTATCTACAAGCA
GAAGGCGGAGGAGACCGGCAACTCACAGACAGAGAGAAGGAGATCATCCGCCAGGCAGCCGTGCAGCAGACCAAGGAGATGGACCTGAG
CGTGGTGCGCCTCATGTTCACAGCCTTCCTCCCTGACAGCACTGGCAGCTTCACTCGGAGACTGGAGCCTGTGGTGTCAGACGCCATCT
ATGATAGCAAAGCCCCGAATGCATCCAACCTGAAAATCGTGAGAATGGACAGAACAGCAGGATGTGTGACGGGAGGGGAGGAGATTTAC
CTTCTCTGTGACAAGGTTCAGAAAGATGACATCCAGATTCGGTTTTATGAAGAGGAAGAAAATGGCGGAGTTTGGGAAGGATTTGGGGA
CTTTTCCCCCACGGATGTTCATAGACAGTTTGCCATTGTCTTCAAAACGCCAAAGTATAAGGATGTCAACATTACAAAGCCAGCTTCCG
TGTTTGTTCAGCTTCGGAGGAAATCAGACCTGGAAACTAGTGAACCGAAAACCCTTTCTCTACTACCCTGAATCAAAGACAAAGAGGAA
GTGCAAAGGAAACGCCAGAAGCTTATGCCGAACTTCTCGGACAGCTTCGGCGGCGGCAGTGGAGCGGGAGCCGGTGGTGGAGGCATGTT
CGGTAGTGGCGGTGGCGGAGGGAGTACCGGAAGCCCTGGCCCAGGGTATGGCTACTCGAACTACGGATTTCCTCCCTACGGTGGGATTA
CATTCCATCCCGGAGTCACGAAATCCAACGCAGGGGTCACCCATGGCACCATAAACACCAAATTTAAAAATGGCCCTAAAGATTGTGCC
AAGAGTGATGACGAGGAGAGTCTGACTCTCCCTGAGAAGGAAACTGAAGGTGAAGGGCCCAGCCTGCCCATGGCCTGCACCAAGACGGA
ACCCATCGCCTTGGCATCCACCATGGAAGACAAGGAGCAGGACATGGGATTTCAGGATAACCTCTTTCTCGAGAAGGCTCTGCAGCTCG
CCAGGCGACACGCCAACGCCCTTTTCGACTACGCAGTGACGGGGGATGTGAAGATGTTGCTGGCCGTGCAACGCCATCTCACCGCCGTG
CAGGATGAGAATGGGGACAGTGTCTTACACTTAGCCATCATCCACCTCCACGCTCAGCTCGTGAGGGATCTGCTGGAAGTCACATCTGG
TTTGATCTCTGATGACATCATCAACATGAGAAATGACCTGTATCAGACACCTCTGCACTTGGCCGTGATCACCAAGCAGGAAGATGTAG
TAGAGGATTTGCTGAGGGTTGGGGCTGACCTGAGCCTTCTGGACCGCTGGGGCAACTCTGTCCTGCACCTAGCTGCCAAAGAAGGACAC
GACAGAATCCTCAGCATCCTGCTCAAGAGCAGAAAAGCAGCGCCCCTTATCGACCACCCCAATGGGGAAGGTCTAAATGCCATCCACAT
AGCTGTGATGAGCAATAGCCTGCCATGTCTGCTGCTGCTGGTGGCTGCCGGGGCAGAAGTCAATGCTCAGGAGCAGAAGTCTGGGCGCA
CGCCGCTGCACCTGGCCGTGGAGTACGACAACATCTCCTTGGCTGGCTGCCTGCTTCTGGAGGGTGATGCCCACGTGGACAGTACCACC
TATGATGGGACTACACCTCTGCATATAGCGGCCGGAAGAGGGTCCACCAGACTGGCAGCTCTTCTCAAAGCAGCAGGAGCAGACCCCCT
GGTGGAGAACTTTGAGCCTCTCTATGACCTGGACGACTCTTGGGAGAAGGCTGGAGAAGATGAGGGAGTGGTGCCAGGTACCACACCCC
TGGACATGGCTGCCAACTGGCAGGTATTTGACATACTAAATGGGAAACCGTATGAGCCTGTGTTCACATCTGATGATATACTACCACAA
GGGGACATCAAGCAGCTGACAGAAGACACGAGGCTACAACTCTGCAAAACTGCTGGAAATTCCTGATCCAGACAAAACTGGGCCACTCT
CGCACAGAAGTTGGGTCTGGGGATATTGAACAATGCCTTCCCGCTCAGTCCTGCTCCTTCTAAAACTCTCATGGACAACTATGAGGTCT
CTGGGGGTACCATCAAAGAGCTGATGGAGGCCCTGCAACAGATGGGCTACACAGAGGCCATTGAAGTGATCCAGGCAGCCTTCCGCACC
CCGGCAACCACAGCCTCCAGCCCCGTGACCACTGCTCAGGTCCACTGTCTGCCTCTCTCGTCTTCCTCCACGAGGCAGCACATAGATGA
ACTCCGGGATAGTGACAGCGTCTGTGACAGTGGTGTGGAGACATCCTTCCGCAAACTCAGCTTTACAGAGTCTCTTACTGGAGACAGCC
CACTGCTATCTCTGAACAAAATGCCCCACGGTTATGGGCAGGAAGGACCTATTGAAGGCAAAATTTAG
HUMAN SEQUENCE - GENOMIC

(SEQ ID NO: 28)

| | |
|---|---|
| TTGACAAGCTGGTTCTAATGAATTCAGATGCAGGACCTAGAATAGCCTAAAACTAAAAACCTTGAAAAAGTGAACAGAAGTTGGAGGAC | |
| TAACACTTCCTGATTTCAAGACTTATTGTAAAGCTATAAGTAATCAAAAAGGTGTGGTATTATCATATAGATAGAGAAAAAGATCAATG | |
| GAACAAAATAGAGTCCAGACATACAGATACAACTGATTTTTAACAATTTGCAAAGGCAATTTAGTGAAGAAAGGATAGTTTTTTTTTTC | |
| AACAAATGCTGCAGAATCAGTTGGAAATCAATAATCTAAAAAGAAAAAACTTCAATCCATATCTTGTACCACTATATGCAAAACTTAAC | |
| TCAAAATGTATCATAGACCTGAATTTAAGTCCTCAAACTACAAAAGTTCTAGAAGAAAATAGAGGAGAAAATCTTAGTGAGTTTGAGTC | |
| AGGCAAAGAGTATGTGGATACAACACTGAAACCACAATGCAGTAAAAAACTGACAAATTATACTTCATCAAATTTAAAATCTTCTCTGC | |
| AAAAGAGGATGAAAAGACAAGTCACAGGCTAAAAATATGTATTTGCAAATCATATATCTGGTTAAAATACTTTTATCCAGAATATAAAA | |
| TGAACTCTCAAAATTCAATAATAAGAAAAAAAATACACCAAAAGTAAACAAAACATTTGAACATGCATTTCACCAAATGGCAAATAAGC | |
| ACATAAACAGATGCCCATCATCATTAGTCATTAGGGTAAAGCAAATAAAAGCACAATGAGATACCACTAAACATATCAATTGAGACGAT | |
| TAAAATAAAAAAGACTGACCATACCAAGGATTGCAAAATATATGAAGGAGCTAGAACTCTCATTTCCTGTAGTGGGAATATAAATAATA | |
| TACTCTTTGGGAGAACAGTCTGCAGTTTCTTAAAAAGTTACATGTATGCTAACTATACGCTCTAGCCATTTCACTTGTAGGTATTTCCC | |
| ATGACAAAAGAAACTATATGTCTATATAAACAGTTAAATAGTTACACACAAATCTTTTAGTGGTTTTATTAATATATCCCAAAACTGGA | |
| AACAACCTAAATGTCTATGAACAGGAAAATGGATAAATAAATTGTGACTATCTATACAATGGAATGCTACTCAACAATAAAAAGGAATT | |
| AACTACTCATACAACAACGTCGATGAATTTTAAGATAATCATGCTCAATGAAAGAAGTCAGATAAAAAATAGTACCTACTGTATGATTC | |
| CATTTATATGAAACTCTAGAAAATGCAAACTAATCTATGACAGAAAGCAAATCAGTGGTTGCTTAGGAATGTGGAGGAGTGAGGAATGG | |
| AAATCACCAACTGCAGACGGCCATGAGAAAACTTTTCAGCTGATGCACATCCTCACTCTCTTTATTATGCTCATGGTCTCATGGGTATA | |
| TATATATATCAAAAACAACTCTCTCTCTCTCTCTCTCTCCAATCCCCCAACCCCATCTCTTACAACAACAATCTATCTCCCTAGTATAT | |
| CTGGAAGCCATCTTTTGACCAAGAGAAAAGCCAGCATGAGGAAACTGTTAACACAGGGACAGAAGAAAAGAAAATGTCTCTGAAGGGAA | |
| GAGAGCCAGAATTCTCATTACATTAAACCTAAAGATTTTCAACCATGTGACCTGATGAAATTCCATAAATGCTTACTCATTTTGAGTCA | |
| TAATTTTACATCTTTTCACCTTAGGTGAAAAGCATCCTAACCAAGATAAGTATATGCTCAGCATTATATTGATAATTTATAATACGCCA | |
| TTGGAAAAAAACTCTCTTCCTGGCATTTAATTATATTACTTATTTCAAAGAAAATGCAGTAAAAAACTGATCTGTATATCTACTGATAT | |
| GTCTATCAGTAAAACAGATAAGTATATCTATGTATACCTACTATGCATAAGCCTTACAGGTCCTACATACACGTGATATCTCTTGTTTA | |
| TACTCTTTGTCTTCCCTTCTTCCTTTTTTTGATAACAGTGCCCCAATTTACGCTTAGCTAACCATAATCTAGCATTACTAATTCATGTC | |
| TTCCAGATGACATAGACCCTCCTTGTCACTAAAAGTAGACATGTGACTCACAGCATTCTAACAAGATAGTAATGGAACACAGGTCCCCA | |
| AATTTACCCTCCCAATCTCTTCTCAATTACTTTCCCCAACAAGAAAAAATGCTACTGCAGGTAAGCAGGGGCATCTAAACCAGGGTCAC | |
| TTGAGGTCACAGGCTCCAGGGTCTCTAAAAGGAAATGAGATACTCTCACTCTTCTCTGTTTTAGCTACTCTCGAGGGCCTTGTTATAGA | |
| AGACTTAAAACAAAGGTCTCAATTTTATGGCTTCCACTGAAAGTTAGCCTGAGGAAACCTATGAATGCCTTCACCAAACCATTACATGG | |
| GCTTATTTGTGTGTGTTCTTGGGAAGGTCAGACTAATCTGGGTAGTGACAGGTCATATGGAGGAACGCTGCACTCATGGAAGAGTTGCT | |
| TGTAGCCCCTGAGGTTAGGCAAGCATTCTCCCTTGCACCCAGGGAGCTCTGGGAGAAGAAAGACTTTTTTTAAACCTTCAGCTGGCTGT | |
| GGACAGATTGCCAGTTGGTTTACTGCCAGCCCACAAAAGCAGAAACCTTTCCCCTGGCAGGTTTGCCACTGCATAAAAGACCCTAACTA | |
| CTTGTCCTGTAATTATAGGTCAGTGTATACAGAGCCTGAGGTCTGAACAGGAAATGGAAAATAATAACTTGCCCTAGTACTACTAGCAA | |
| GCCAGAGAGGAAAACTAAGATAAACACTTCAAACAGGTCTTTTGTTGAAATAGAACTGTTAGAAGAGCCAGGTGAAAAACTTTTCTAAA | |
| AGTAATAAATTCACTCAAGAAGATATAATTAAAGATTAGGAGTTAAAATATATCATAGATGCTAGAATGATAAAATATACATATTTTAA | |
| AATAGGTAATGACTCCATCTGTTTCTGACAGTATACCCTTCCTGGTTGAAAATAACTAAAATGATAACTAATGCATAAAAAACTGAATG | |
| CTACATAACATATTTTTAAAAATTAATACTGCTTAGCCAGCATGAAATTCAGGAATCTAATATTTCCAAAACTGAAAAAACTGGAGGGA | |
| ATAGCTGAGAAATTGACTTAGTCTGTTTTGTGTTGCTATCACAGAAAACCACAGACTGGGTAATTTATAATGAAAAGAAATTTGTATCT | |
| CACAGTTCTGGAGGCTGGGAAGTCCAATATCAAGGTGCCAGCATCTTGTGAAAGCTTTCTTGCTACATCATCCCATAGTGAAAGCAAGA | |
| AGAGCAAAAGAGCAGACACTCAAGAGAGAAAGTAACCTACTCCTACAATAATGCCATTCATCCATTTATGdGGGTAGAGACCTCATGAC | |
| CCAATCAGCTCTCAAACGTCTCACCTCTCAACACTGTTGAATGGGGAGTTAAGTCCCAACACAGGAACTTTGGGGGACATGTTCAAACG | |
| ATAACACTGCCTATCTAGTCCTTGTCACTGGACAGAAAAACAACAAATTCTCACCTGTGAATTCATAACCACTTACACTATATGTGATG | |
| ATCAAAAGCACACAAGTAAAAAATTTTATTTAAGGTTTTTCCAAGTTAATAGTATACCAGATAACAGGCAAAAAGAAAGCACAATATTC | |
| CTTACTCAATTTTAACCTAAGCTTCAAATAATTTCCAGAGATTCTTTCAAGAAACGTAAGCAATTTATTATTTATAATTCAGTTGACAT | |
| ATTAAACAGCATATGAAATATAGCTGAAAAGAGAATTGATAAACTAGAAGGCAAATATAAAGAAATTATCCAGAATGAAATCCAAAGAG | |
| ATAAGAGATATTAAAAGACACAGAGATTAGATTGAGAAGATCTAACATATACCTAATTTAAATTCCAGAAAACAAGTCTAGGGAAAATG | |
| AAAGAGAGGAAATGTTACAAAAGATAATGGACTAGGAATGTAACTGTTGAAACATCAAATCTTGAGACCCAGGGAGCCATATAACATGC | |
| TTCCAAAATCCAACCTTATTATATTAATATACAAAAACATTAATATTAATATTATATATTAATGTTATTGTTAATATAATCATTATATT | |
| ATTAAACATATAATTTATACATAACAATATATTATATATAAAAATGTATATGATATATAATATACAATAGTTATAATTTATATTAATTA | |
| TATATTGACTTCTATATAATATAGAATTATATAACTTATAAATTATATATAGCTTTATATATAAATCAATAATTATATATTGATGTGTA | |
| CAATTATTATTTTATATTATTATTAATAATTATATAATTGGTATTATATAAATATTAATTTTATATTGCAATAGTTATAAAATAATAAA | |
| CATATAACATTATTAATATAATAAATATAGTATATTAATATTAATAACTTGTATTTATTACAATATATTAATAAAATGACATAATTTCA | |
| CATTTCTTTCAGATTTCTGTAAAAGCTTATCAGGAGTTATATATATGCAAAACCATATTTATCTTATTAAAGAGGCATGTATAAGTGTC | |
| AGACCATCACTTATGAATAGATTTGACACCCAGATATACAACCTTTCAGAAACAGAAGCAAACCTCCTCCATAAACAAGTTTAAATTGC | |
| CACCTGTCTCAATGATGTCCAGTAACTAGCCTTATTTCACTCAGAACAGTTTTGTCGGCTTCATATAGCATGACCAATCACCCTGGTTT | |
| ACTAGAGCCTGAGGGTTTCCTGCGCAAGGGACTCTCTGTGCTAAAAGCAACATAGTCCTGCATCAACCGGGATGAGTTAAGTCTCTATT | |
| TCCATGCCTTCATAGAAATCATTAAAAGATATCTCTCTGTTATGGGCTGAATAATGTCACCCACCCTGAAATTCCTATGTTGAAGTCCT | |
| AACCCCCAGTACCTCAGAATGTAACTGGATTTTGACATAGGGTTTTTAAAGACGTATATAAGTTAAAATGAGGACAGTGGGATGGCCCC | |
| TAATCTAATATGACTCATGTCCTTATAAGACGAAATGATTTGGACGTGGACACACAGCGACAACCACATGAGGACAGCGAGAAGATGGC | |
| GTTTAGTGTATTTCAGCTGATGATTATAGCCTAATCCTCCCCCTCTTCTTGGGGCCTCTCTTCTCTGTACTCTTCCTCCTCATATCTCA | |
| TATTTCTCTTCTTTAAGCCACCCAGTCTGAGATATTTTGTTTTCGCAGCCCTAAACAAACACATTCTCTACGCTTTATTCTCATTTTTG | |
| GTTTAGTGTATTTCAGCTCATGATTATAGCCTAATCCTCCCCCTCTTCTTGGGGCCTCTCTTCTCTGTACTCTTCCTCCTCATATCTCA | |
| CACAGGCAGATTTCTTCCTGCCTCCTCACCTACATTACGAGCTCCGCACAGTCTCCTTTCTTCTGGCTCCGGCTCCCTCATTAATCTTT | |
| CTGTTATTAACGCTATTAACTCGTTAAACTATCTCTCCACCTTTCCAAACTTCCATTAACTCTCGCACTCCATAAGTGTTAGTCAAATA | |
| AATGAAGAATCACACTAACTCCTCTTTCCTTCCCTCTCACTCCAAACTCTTGGTGAGATTCTTTTGCACTTATGCCCTCGTTTCCCCAT | |
| TCTAATCACTCAGACTAGAACACTCAGAGCTATCTATGAGTCTTCCTCTCACTCAAACTGCATATATAATCTATAAACAGGTCCTGTTG | |
| CGTATCAGTGCTATTTGAGAGTTAATAAAGTTCCCTTTTCCTCCAAGGACAGCGAGCAGAGTGAACAGAAGAAGACTGCTTTCTCCCCT | |
| TGGCTGCACAGCCATGCCCCGAGGCCGGGCTTAGGATTTTATAATTCACAAAGTCAACTGTCTCTTCCCCTCTCCATCCTTTAGGAACT | |
| GTCTGCAGTATCTCACACTGTTGAGAGCTCTCATACCCTTTAAACTCTCTCTTCTCTGCCTTTCTGACCTTTCCTCCCACTTTTGATGG | |
| CACTGCAGGAGTTTTTAAGCATCTCTCTCTCATACCAACCCCAGAAATAAAGTCATTCCTCGAATTCCTGTACTTACCCCCTTGTCTCC | |
| TAACTCTTTGACCACTAATGTTTTAGAAACACTCTCCTGACAACTATCGCATCTTTGCAGTTGTTTCCATTTCCAACGCCATGTCTCTC | |
| TGAGCAGCAGCCTTGAGTTTTCAATTCCATATTTATTAGTCATTTCCATATGGACATCTCACTAAATCTGTCATACTAAACATGTCTAA | |
| AATATGTATGTTTACATGTTACATGTATGTTTGGTATGTGGCAATGCATTACATTCCCACAGATTTTTTCCCAGTTACACTCTTACTAT | |
| TCTAATCACTCAGACTAGAACACTCAGAGCTATCTATGAGTCTTCCTCTCACTCAACTGCATATATAATCTATAAAACAGGTCCTGTTG | |
| ACCTCATCTTCTAAAAATCTCTTACATCCTCCTTTTTATATCATTGGATAATAGTTTAGTCTCGTGGTTAATATCATCCACACTGGAGC | |
| CAAATCTGTTAGGTTCAAATCCTCACTCTCCTTCTATTTACTAATTATGAAACCTTAGGAAAATTATTCAACTTTTCATCTGTAAATAT | |
| GGGGATTATAATGGTGATCCACTTTATATGACTGACAACCAAATAAGTGAATACGTGTATAGCAGGTATTAACCATATGCTTAGCCGAC | |
| CTATTTCAACCTCCTAACCAACCAGAAGAACTGTAGTTCCCTGCTCTCTTGCATCTAAGTGGGGCCATGGAATGTGGGCAGAAGTGTTG | |
| TGTCACCTTTACTCAGAGGCAATTAAAAAGAAGACATACTTTTCCTATGTTCTCATTGCCAATGAACACACTACACAGATCTCAACCTC | |
| AGAATTCTCTCCACACTGGTATTTCCAAAATAGCTTCATGAGAAGGGAAATTTGTGTAGTTTCAATGATTTAGTGATGCTATGGTTTGG | |
| CTATTTGTCTCCTCTAAACCTCATGTTGAAATGTGACCCCAATTTTGGAGGTGGGGCCTTATGGGAGATCTTTCGGTCATGGAGGTCCA | |
| TCCCTCACAAATAGATTGATGCTATTCCCTGCAAGCAGGAGAGCATGAGTCACTTCTCACTGCATTAGTTCCTATGAGAGCTGGTTGCT | |
| AAAAAGTTTGGCATCTCTCCCCTCTCTCTCGCCATACGATCTCTCTCCACACACCACCCGCCTTTGCCTTCCATTCAGTGCAACCACCC | |
| TGACGCCCTCACCAGACGCATATCCTGCTGCCATGCTTCTTGTACACTCTCAAGACTGTGAGCCTAATAAACCTCTTTTCATTATAAGC | |
| TACCTAGTCTTGTGTATTCTGCCATAGCAACACAAATGAACTAAGACAAGTGACAAACACAAAATAACTTGTCTTTTAGACAATATTGA | |
| TGTCTGAAATTCCACATTGAGGCATTTTTTTCCTCTTCGTAGCCTGATAATGGCAAATACCTTCTGACACCTTAGGATGGATGAATTTG | |
| TAAGAGATATTCCTGGAAGCAGGTTTGTACAGTGTAGAGAAAATGTTGGGTAACAAAGCAGGAATGGAGGAGGGTTAATCAGCAAGCCA | |
| AAAAGCTTTTATAGTTTTGCCTGCTGTGCTGCATGGAATTACATCCCCCCAAAACATATGTTCAAGTCCTAACCACAGGTACCTGTGAA | |
| CATGAGTTTATTTGGAACTAGGATCTTTGCAAATGTTATCAAGTTAAGATGAGGTAATACTGGATTGGGGTGGGCTCTAAATCCAATGA | |
| CTGGTGTCTTCATAAAAGAAAGGTGAGGGGAGAGTTGAATATAGCGACACAAAGGAAACACGGGGAGGAGACCATGTGACAATGTAGGC | |
| ACAGAATGGACTGTTGCAGCCAAGCCAGAATGGCAAGGATTCCTGCACCTACCAGAACCTACGAGGACGCAAAAAATTCTTCCCTAAAA | |
| GCCTTCAGAGGGATCATGGCCTTAATGGCACCTTGAGTTCAGAATTTTAGGCTCCAGGACTGTGACAGAATAAATTCCCGTTGTAAGCT | |
| GTTCAGTCGGTGGTAAATTTTTACAGCAGCCCCAGGAAAATATGCCTGCTCAACAGGGTACCCTGAATTTTAAGTTCATTTTATGGAAT | |
| CATTTTGAGTTTGGAGTTTCATTTAATCCTTTTCACTAAAATTAACGCTCTTCATTTTTTACTCCCAAATGTCACACTGGCTCTACTCT | |
| TATAACAATTGTATTTATCTATCTTTTGGTAATGGGGCTGCCACCCCTTGCAGAATGAAAAGTAGAGTGTGTATGCTCACACCTCCCTC | |
| CCTCCCTCCCTCTCTCCCTCCCTCTCTCTTTCTTTCTTTCTCAGAGTATCAACTCTGTCTCCCCCAGGCTGCAGTGCAGTGGCCCATCT | |
| TGGCTCACTGCAACCTCTGCCTCCAGGGTTCAACCGATTCTCCTGCCTCAGCCTCTCGACTACCTGGGATTACAGGCGCCCGCCACCAC | |
| GCCCGGCTAATTTTTTTGTATTGTTAGTAGAGATGGGGTTTCACCATATTGGTCAGGCTGGTCTCAAACTCCTGACCTCAGGGATCCAT | |
| CCCGCCTTGGCCTCCCAAAGTGCTGGGATTACAGGTGTGAGCCACCGCACCCGGCTCACAGAATTTCTTTAAAAAGTCATTGTGTTCAG | |
| CTTTATGGATCCTCTAAATTCCAGCTGCTCAGCCTCCCGGAGCTAAAATGAGATCACTTAAAGTCGACTCTTGGGGCCTCCAAGGTCGC | |
| CTCGGAGACTCACCTCATAGGCAATCAATGGATCGCTGGGTTTTTCTGTGTGTGGGTTTTTTTTGTTTATTTTTTTTTTCCACTAAAAA | |
| CACTCCACCAAGAAGGTTTTTATAACCTACTGCAGGAGGAGGATGGAGAGTGGGCACGAAAGACTAGGATGAACATGCCATAAGCAAAT | |
| CTAAATGCCACTGCAATATTGCCTGGTGAGGACCTGATTACTGATGTTTTAAAGCTAAGCTTTCAGTTGTCACTCCACCCAGTAGTGAA | |
| ACAATGAGCTCTAAAATATATATTTCGGCTCAAGCTTTCTTATGTGGGGAGGTAATCCACCCGAAGGTATCCCCAGCCTGTACCTAATA | |
| CAGTGCCCAGCACTAAAGCAGCTCAGATGCCAGTGAATCGTGGCCACTCGGAGGCCTGTCAGTGGGTGCCAGTACCGGTCTCTTCAGAG | |
| AAAAAGAAAACTCCCCTCTGCCACATCAGTATTTTATGAGCTGTGAACCAAAACCATTGCTACCACCATCACTATAATTCTATCCACAG | |
| TAATTATCATAAAGGCCTAACAATGCCTTGTAGATGAACATTCTGAGTAACTGCTCTATAACCAGGAGATTTAAGACCGCACCAAAAAC | |
| CAGTAGAGGGTTATACTTTACTGGGCACAAGTCGTTTATGAGAACGAAATTGTAGTTTAATCTGTGAAGAGATGTGAATGTAACTGAGA | |
| CACGCTTAAATGGAATATACAGATGAGCTTTATTTTTATATCTGGCATGCTTGGATCCATGCCCACCCTCCAGCTGCTCGGGCCTGCCC | |
| TTAGGGGCTATGGACCGCATGACTCTATCAGCCGCACTGCCACCGCCGCCGCCTCCGTGCTGCCTGCGTTCCCCGACCATTGATTGGGC | |
| CCCGCAGGCGCTTCCTGGGCTTCCCTACCGGCTCCAGCCCTTGGGATTCGGGAGCGCCCTGCTAGGAAAAGCCAGAGCCCCGCAGGGGC | |
| CGCCGCGTCCAGGCCGCCTAACGCGCGCCCCTCGCCCGGCGCCCCGAAGCGGCCCCGAGGGGCGGGAGCCCAGGCGAGCGGCAAGGCCG | |
| GGCCGGGGGCGCACAGCCCCCCTAGAAGTGCGGGCTTCCCCCACCCCCCCCAGCGACCCTACCTCCCGCCCCCGCTGCGTGCGCCCGTC | |
| TGTCCGTCTGTCTGTATGCTCTCTCGACGTCAGTGGGAATTTCCAGCCAGGAAGTGAGAGAGTGAGCGAGACAGAAAGAGAGAGAAGTG | |
| CACCAGCGAGCCGGGGCAGGAAGAGGAGGTTTCGCCACCGCAGCGGCCCGGCGACGCGCTGACAGCTTCCCCTGCCCTTCCCGTCGGTC | |
| GGGCCGCCAGCCGCCGCAGCCCTCGGCCTGCACGCAGCCACCGGCCCCGCTCCCGGAGCCCAGCGCCGCCGAGGCCGCAGCCGCCCGGC | |
| CACTAAGGCGGCGCCGCCGCCCGGCCACCGCGCGCCCTGCGCTTCCCTCCGCCCGCGCTGCGGCCATGGCGCGGCGCTGACTGGCCTGG | |
| CCCGGCCCCGCCGCGCTCCCGCTCGCCCCGACCCGCACTCCGCCCCGCCCGCGCTCCGGCCTGCCGCCGCCTCTTCCTTCTCCAGCCGG | |
| CAGGCCCGCGCCGCTTAGGAGGGAGACCCCACCCGCGCCAGGAGGCCGAACGCGGACTCGCCACCCGGGTAAAGCCAACTCGGGCGAGT | |
| GGGGCCCCGGCAGGGGACGCGTGGCCCAAGTCCTCCCGCCCAGCCCTCCGCACCCCCTCCAGCCCCACTCGGACTTCCTCATTCCTGCG | |
| CTAACCGCTGGGCTAGACCGTGGGAGGAGGTTGACAGTAGCTGAGAGGCACATGGGATTAGCGACAGCGGGGAAAGACACATCCGGACC | |
| TCGCAGGGGCTAGTCGGCCAAGGGCCGCGGCCGCCCGGCGGTCATTTCTCTTCACGTCCCTCCGCCCGGCAAGGAACGCGAACCGGAGG | |
| GGAACTCTTACAAACTTTTAAAATCCCCAACCCCAGCCCCACCTGGGGGATGGTGAGAAGGTTGGAGTGCGCTGCGGCGCGGAGGAGAC | |
| AGGGAAGGTGGTTGGTGCACTGCAGAACCAGATTTCACCTAGGGCGTTCCATGAAATGAAAGCAGAAGTGCTTAATCAGTCCTCTTGGC | |
| TGGGAAAGCCCTCCCCAGCTCCCCAGAATTGAATAGGAGAGTCTATTCATCCCTCTCCTACTTCTAAAAGAAACCTGGCTCGCTCTCTG | |
| TCTCTCTCTCTCTCCCCCCTCCCCCCCTCCGTGCGCCCCCCCGCGCGCAAACACACACACACACACACACACACACACACGATATAGTC | |
| ACCTGCTATAGGACTTGATTCTGATCCTCCGGGGCCTGGCATTTGAGAGAGAAAAATAATTACCTACCTGGATACTTAGAGCACTTTTT | |
| ACCTGCTATAGGACTTGATTCTGATCCTCCGGGGCCTGGCATTTGAGAGAGAAAATAAATTACCTACCTGGATACTTAGAGCACTTTTT | |
| TAAATGCAAGTTTTTCTATCCTCCTTAAAAACAGACACCAGGGAATGTTTGCCCTGACTTGCTGGTTTATCCTGAAACTCAAATCGGAC | |
| TTAATGATACAGGATTTAATTATCAAGATTGACATGTTCATGGACTTTGTTAAGTAGAGGTTTTCAGCATTACAGTTTATGAAATAACC | |
| ATATTTAACTCAAAGATTTTTTTATAAAGCTCATAATCTGAAAAAATGCTTATGGTATTACTAAAACATTTAATAGCTCTGGCATCAAA | |
| ATATTCTACATACTGGTGATCTTTTAGTAAATAGTAAAGGTTAGGGTCAAGATATTCAATAATTTTTTTCTAGCAGAATCCCACAACTG | |
| AATATTGTCAAGCAGTTTAAACTGCATTCGTGTTTGTTAAAACTTTAAAAGGGAAACTTAAAACTAAGTATGTTGTTTTTCTGATTTTT | |
| AATATTGTGCTTTCTAGAGATGAATCCTTTTACTGTTTGAGGCACTATACGAATTATGTATTTTAAAGTATGTATTTAAATTTGAAGCA | |
| ATGTACTTTTTTGAGTTTATAAACTTGGGAAGACAGGAAATAAAAAGATTTGTTTCCTGTAGAATTTTACATTCATGATTTAATTGAAT | |
| TGCTAAAATGCACAACATGTACGATTAAATGGAGACTTGGAATCTGAGATTATTCATCCATATTGATACACCTGCAAATTCCTAAATGC | |
| CCTCCCCTGCTTGTTTAGATAAAATGTCTTCCTGGGCTGCAGCCTACTGGACAAACTTGAACACAAAGGAACACACGTTATATGTACAT | |
| TTAATACTGGAACCGAAAAGCTGCTTGCAGTGAAAAGCAAACCACTTCTAAACTCTTTGAGACTTTTTAAAGAAGGTAGTATAATCCTT | |
| TTAGGGGTTGGTGGTCATGTGAAAATCTATTATCTTTTTGCTGAAAATTCATTCTTATGTTAGGCATTGGCACTCACTTGTGCTCAGTA | |
| AATGCCCGTGTTTTTCAAACCCAGATAGTAAAATACTGCGGGTATACATACAAAATAGTCCTTCCCCTTTGTAAGCATTGCTTTGATCT | |
| TTGCACTTCCTTTTTACTCTACCTCTTAAACAAGATCTGTGTTGATTGAGTTGATTAAAGCACAATTAATCTGAAATAGGCAGAATTTT | |
| AGATTTAGTGATTATATTCCTTACATTTCTGTTGTTACTGTTCCTGGAAACAAATTGAAGTAGTTTGAGAAACTAATATTTATGCAGGT | |
| TCTTTTAACTACACTTTTAGACTTGCAGATAATTCATTAAGTAAGTTACTACCTTGACTTTCAGCGATTCCTCATTGTGGTAGATACGA | |
| GGTCTAGATAGGGAATTGGCAGCTACATGAATGTTGGACTATCATTCCATCAGCAAGACCTTATTTTTACCTAATTTATGACAGGTATT | |
| TCTCTGTTCAGAAGAGAGGTGAAAGTTCTGGCTTCTGGGGGGAAGTGGTTACTTCATAACCTTCAATTGGTTTGAACTTGAGAAGTAAG | |
| AAAAGTAGTCGATATTTTCAAACAGTAAAATAATTGTATCTGAGTTGCTGTATGGATTTTTGATGAAGTACTCAAAATGTCATCTTTTG | |
| CATCTTCGCGTCTTAGTAGATGTGCTATTCTGAGTATACAATTGACTGCAGTAGGAAGTAATATCTGGAATCCACAGTACTGTTGTATA | |
| ACCAATAGAGTAAATTCTCAGATTATGCTCTTCTGGTCCACAGATTGATCAGAAATCACATTAACAGGAATGGATCTCTATGCAAAATT | |
| TCTCATTAATTTTCTTACTTTTAGTACGAAGTTTGTTTTATTCGGTTGGTCTGTTGGTTTTCTGTTTACAAGGTGAATTGTCTACAGTA | |
| CTCTCTTTGAATTTTGTCACTACTTTGGAGACAGACAGTTTGGAAGAACTGAAGGTACTCAGCCTCAGGGAGAAAGGTTTGAACGTTTT | |
| TTTGTGTCTAATCAGCATTAATTGATGACTACGTCTTGGTAAACAGTAGACCATACTGGAGCAATTTACTCTGCTTTTTGCCAATTAGT | |
| TCTCATTTGCACTCTTGCACCTCCCATAAATACTGAACTCACTTGTAGTGTTCTTAGAGGTCAGCAATAAACCATCAAGAGCATATGAT | |
| CTCTCTTTGAATTTTGTCACTACTTTGGAGACAGACAGTTTGGAAGAACTGAAGGTACTCAGCCTCAGGGAGAAAGCTTTGAACGTTTT | |
| GGCATAGTCCTGCAAGTCAGTTTGGCTCCTTGACTGTGAATTTTTTTAATCAAAAAAGATTAATATTTGAAACCACATAATTTGGACAG | |
| TTAATAGTTCTGGTAACATAACCAAGCCTTTTATCTTTGTAGTATTTCATTAATTAGTACCAGTGCCTCACTTTTAGAAGAAAAAAAAG | |
| ACAAAAAAGTAGTAACATTCTTTTAATCATQATCAAATTGCTTCATACCTTTGGTCTCTGCTGTATTAGGGGTTGTTCCTCGGTCCAAA | |
| TTAATATACTGTCTTTTACATTTCCGGAGGTTGTTGAACCTGGCCTTGAAGATCACCCAACACTTTAGAAAATAAGTTGTGCAAATGCA | |
| TTGCTACACCTCAAATAATTTAAAGCCTCACAGTTCTGTACACAATAACACTCACATACATCTGATTTGTTAAAAACATTATACAATCT | |
| CAGAAGATAGATTATGGATGAAAATATTTGTTTAATTTAGAGTACTAATAATAAAAATAGTGCATTTTACATTTTAAAAAGAAAACAAT | |
| TTTTATTCTCTCATATTTTCTGTAGTAACAAACACAATTAATTATCAGTAATACAAATTTCATCTCCTTTAATTTATTCCCTGTTTTTG | |
| AAATTAGAATAATGATATATTTGGTTTAATTATCCATTACAAATTGCCTTATTATTTTTCATTCATCTTTCACCATTAATCCAGAAACC | |
| TTAAAATAGCTTTTATTTTGACCCATGCCTTCAGTAGATATCAACACACTTGAGTTCATCTATGGATCCTATTACTTTAAACTGTGACA | |
| AATTTTGACTGCTTAAGCTTTAGAAAAGAATTTATTCTTTAAAATAGCCAAAATTATAAAAAATGAAATAAAGAAAAAGCTGTTTGAGA | |
| GAATCAAATACTCTAGTTTAACTAAAAATAAATAATATCTAATCAAACTGACGAAAGAAAATATCTCATGCTTAGCATTGTACTTGTTG | |
| GTACATAGTAAGCAATCATTAACTGTTAAATGAATGAATGATATGAAGTTGTTGCAAAATTTTTTTTTAAATGGACAGCCATCTTCTAT | |
| AAGGAACAAAAATTACATTATTTTGCTTTGCCTTCTGGAAAGAAAGGAGCACAAAAATAGTCCTTGTGTCTTAGATATTATGCAATCTT | |
| ATCTAAAGACCACTTGAAGTTTACTAGAACAAATAATTTATTAAAAAGTATCTGGCGCCTTCCAAAAGGGGAACATTTCATTCTGAAAG | |
| AGGCATTTATGAACTAGAAAAAAAATGCCTTTTAGTTTCAAAGTTTTAGAGGCACCAGAGCAAGATTATAAAGTAATTGGAATGATAAA | |
| AGGTGCTTGTAAAATTGAAAAATCTAATTTTTTGCTGAAGTTAGAATTTCTGTACACTCCATTGAAAACTGTAATATCAATCTTTGTTG | |
| CTCCAAAAAAGAAACAATGCTACAAGCACTGCTTGGTAATCTAAGAATTGTTAACACTTATCTAAATTAATCAACCAAAAAGTTAAAAC | |
| AAAATATGTTATTTTGTTATGTTACCTCTAGAATTAAACACCTATCACCCATAAATCTGTGACTGTTAAAAGTTACTAGATGGCAAACC | |
| TCAGGAACAAGCAGGCAGTTAGCAGGGTTTACTGTACTTTCCACTTTCATACTTTAATGATTTTTCCAACACTATAGAAGCTGGGGCCT | |
| CATCCTAGCACAGTTTGTTCATTATTTTTCAGTTGTAGCATTTTTTAAAAGTTCGCCTTGAATTTCTGAATTGAACCATTTATTTTTTT | |
| GTGGACACAGTTTTTTAAATGAAATATACCATTTAGACTAGAAAAGAGCAAAATGCCAATACTCCCTGTCCTAGTGTCATTGTGCCCCC | |
| TGCGAACCCTAATGCAGTCCTGTGGGAAAACAACCATTTTATTGACCGCTGCTCACTTCAGTTCATTCTGCTTTGCTGCCACTTAGTTC | |
| TGGTTCTGACCCTGATCTCCCCTCACATCACTCACAACAAACATTCAAGTTTAAAAAATTAAGTTAAAAGTTAGTTACATATATATGTA | |
| CTAATGTGTATATTAAATATACTGATATATATTAGTTACATATACAGATATGGCAACAACAAAAAAGGTTATATATATACATATTTGTA | |
| ACTAATATGTATATTAAATATAATGATATATGTATAATACATATTTGTAACTAATATAATTATATTTAATATATATTAGTTACAAATAT | |
| TGTATATATAATATTTTTGTTGTTGCCAGCAAAATGAGAAAGAAATTAAATGTTAACATCTATAACAATTGCACTGTAAAAGCCGTCAT | |
| TGTTCAAAACTACTCCGAAGATTAATAGCGGCAGTGTTTGTTAAAATAATAAAATAATCCAGGCACTCTGCCTGTGACTTCCCCATGAG | |
| ATGGCTTCCAGAAAAAGATAAATTTTTGTGTGTTCCCTTACGAGTTCAGTCTGCAGCTTCTGGGCATGAAGAGAAGCATAAACAAAACA | |
| AACAAGGCTCTAACTTTGTTATAATCCTTACGCTACTAGTAGAAATGTATGGACAATTCATGCATTTCATATTGGAAAATAAAGCTATT | |
| AGATTTTTCTTTCAGATGAACCTTTTTTTTGTAATTTATTTTTTATTTTTTATTTTTTTTCAGTAGAGATGGGATTTTGCTCTGTTGGC | |
| CAGGCTGGTCTCAAACTCCTGACCTCAAGTGATCCACCCGCCTCAGCCTCCCAAAGTGCCGGGATTACAGGCGTGACCCACCTAGCTTG | |
| GCCAAGGTGAACCTTTTTGATAAATGATTCTCACTAATATTTCTAGGATTTACTTTTCACTAACTTGAACTAAAGAAAGTATAAGACCT | |
| GCATAAAGTTGAGCATACCTCTATAATCTCCCATTTTAAAAAGCAATTTGTTTTTAATGACTTTTATTTGTTAAAAGCAGTTATTTCAA | |
| AAAATTATTTTTATTATTTATCATGTCATTTGGACTCAAAATACTATACCATATTTCAGTGTTAGGGCACACCTATCCCTCTTTATCCC | |
| AGACGTCAATTCTACCATATAACAAGTATGTAAAAAAGAAAACATAAAAGTGATGACAGACTCCCTAATCAACCTATTTATTGGTTCTA | |
| AGCCCAAGCCTGCAGTTCATTTAAATAGTGAAAATAGATTACTTAATTGTCTTAGAAAATATGTCTTCCATGACAAGAATTTTAGGAAT | |
| GACAAAATAACTATTTTTGGATGTTTTAACACAAATTCCCAAATCATGTTTTATTGATATTTGAAGAACCTCTTTGTCTACCAGTTCTT | |
| AAGTATAGGCTTATTTTCTAAGGCTTAAACATGTATTGGTGTTTGACTGTTAAGACTTGATCTATAACCTTCAGTGTCTTTTTCTTTTA | |
| AATGCCGTATGGACTTGGTAATGAGGCCACACCAGAATACCTTACAATTTATAGCATCTGCATATAATACAAGAACCTTCATGATTTAT | |
| AAAATTAAAATGATGATGATGTGGAATGATGGAAGAAGCATGTGTGGGTGGCTATTTATGTAGAGGAAATTAGAAGTTGAAAGCAATGT | |
| AGACGTCAATTCTACCATATAACAAGTATGTAAAAAAGAAAACATAAAAGTGATGACAGACTCCCTAATCAACCTATTTATTGGTTCTA | |
| AGCCCAAGCCTGCAGTTCATTTAAATAGTGAAAATAGATTACTTAATTGTCTTAGAAAATATGTCTTCCATGACAAGAATTTTGGAAAT | |
| GGGAACTACTTTTCAGAGGAGATGGCTTAAAATGAACCAAAAATATAGCCATTTCTCTACCCTATTCATTTAATGCATTCATTTATTAT | |
| ACTGCTAATACATGATAAGGTGAATAGGTTAAGCATCGTTTGTATATGTGTATGTGATTTTAAACCTGAGTCTTTTGAAAGATTGGTCA | |
| AAGCAAAGGATAATGAAAGCATAAAGATTTAGGGGGAAGAGTCCTCTATAAAACTAAGACTTAAACTTTGTTATCTACTTGAATGTGAA | |
| GATAGAATAATCTAGAAAGTGAATGAGAATTATAAATAAAAGGACAAATCATATATCTCATGCTTGACGAATACAATGAGATTAATTGT | |
| ATATCTCATCTCATGGAGGGTCATAATGAAAGAAGTAAGAGAGATTGCCCTTGGATAGAACCTTTCGGCAGACATTTCATAATGGATTA | |
| TCATATTTTTGCTAAACTGGTAAGAATGGACTTTTCACCACATTATGAAAAAACACTTTAGAAGTCAGTTGCGATCAAGCAAATATAAG | |
| TACCTGGGAAATTAAAATCAGCACAGCAGTCCAACAATAGTCAAATGCATTTACTCACTCTCATACCATATCATACAAAATTAATTTAA | |
| AGATAATGTCTTTATGGTGTCTCTTTATATATTTACTCGATTTCCCATTTAAGACATTTAACCAAATTTAAAAAGCAATCCTTAGAAAC | |
| TTTAAAAATATTTCCTATTTTAAACCATTACATAATCATTTACCCTGCAGTTACAATTCTTTTTTTGTGATGGGTGGGGAAGTATCCCA | |
| TTTCTCTTAGAAAGTCTTCTGTAATAGCCAAATAGGAGTCAATCCAAGTTGCTTAGAGAGGTAGTTCAATCCTGTGTTGAAATAGATGA | |
| CTTTTCGACTAATTGTGCTATATTTACTGAGTATCTGTTGAACTGTACCAGTCCAGTCATTCAAGGCAAGCTAACAAGAAAATTTTATT | |
| GCTGAGGCACTTAAGTATCTTCACATCTATATGCCAATATTAAATTTCTCATCATCCTTGATCCCCACCCCTTTGCACAGATTGGTATC | |
| CCCAGCACATTAGGGAATGCGGTTGTTAGCCTGTTAGGGTCCATTCAGTAGATTTCTCATGCCAGAAATCTAGAAGCCCTTCTTATACA | |
| TTCAGCTTTCTTCTTCAGTCAGTCACCAAGTTCTATCAAGGCAGCCTCCTCATCTCCACAAATCCATCCCTTCCTCGTTGATTTCTAGA | |
| TTTTATTTTTGCATTGTCTCCTGCTTCTAACCCATCGTTCATTTCGCATAATGGTCAGCTTTCTTAAAGTGTAATCGGATCATGTCTCT | |
| GCAGTGAATCCCAGTTGTCTTCACAAGCTGATTCTCGCACACCTCACCCCCTTACTCCCTCCGGTGTTCCCCAACCATCCCCTAATCTC | |
| AAATCCTCCACACCGACTTTCAGTTTCCTATCCACAGTTTCTGACTTTGAATGCCTTTCCTTATGCCCTCCCTGTATGAGAAGTGCCTT | |
| TTCTTTCCTAGCCATCCTGCTAACTTCTAAACCCTTAGGCCTGGTTTGAGCATCACCATTTCTCTCACTTGCCTTTCTGTTTTCTCCCT | |
| AATTAACTGTGTGGCTCTGAGCAAGTTAATTAACTTCTCTGAGTCTTATTTTTGTTCTGTAAAAAAAATGAGGTTTGACAAAATGAGCA | |
| TTCATCTTATTTACTTTTTATTTGCACAGCACCTGGTCTGTTTGCTGAATAACTAAATACCCAACAAGATATCAACAGACAGTGCTGCC | |
| AATTAACTGTGTGGCTCTGAGCAAGTTAATTAACTTCTCTGAGTCTTATTTTTGTTCTGTAAAAAAAATGAGGTTTGACAAAATGAGCA | |
| TCTCTCTCTAAAATTATGTGTTTCTTTGATGTAATTGTATTAATTGGAAGACTTCAGATTTTCTTGCCATATCAAGATAGGTCAACTTA | |
| AGAAAAATGCATQTGAGTAAGTACCAAGCCAATTCTTGATTATCAGTATGATAATATGAGGAACTGCCCATTTTGTCGATAATTAGTTA | |
| TAATTTTTGGTCTCAGTGTAGGTTCTTTCTCAAATCCAGATGTTTCTCGTGTTGCATTTTTTCATTATTATTCAGTCTCTCGCATTCAT | |
| ATTTCTCCAAAGAAATTTATGGAAAACTTGAGACAAAAATTGAATGTAAAGTTTAATATTTAAATGCTAGATTGATTCAAGGTCTCACT | |
| AATTATAACTATATTTGGAGGGAGAAGAAGGGGAGGTGGTGGTAGTATGAGCTGAATCCTCCTCTGTTCCTTCCTAGAAGGAACACAAC | |
| GTCCTACATCAAGAAAAAAGACAGAAGCATATCTTGGAGATATGAAAAGAACCACCAGGTAAACTAAAAAGAAAAAGAGTGTTAAAAAG | |
| TCAGCCTTTAAAAAGCACGATTGGAGATGAGGAGGGGTGGAGTAGGGGAACCATTCCTTGTTTATACAAGCTCTTCCATCTTCGTATAG | |
| TGTGATTTTTATTTTTTTATTATTTTTTCVCTTCCTTTTTTTTGACCTCCAGCTTTAGTATAGTACAGTTGACAGAATTATATATGTTT | |
| ATGGTACACAAGATGATGTTTTGATATACATATGCATTGTGAAATGATTAAATCAAGCTGATTAACATAAGTAGGGTTTTATTTCTTTA | |
| ACAGTGATTTATTTGAATATATGTTGAGTCCTTTCTATGTGCCAGACACAGTTCTAAGGACAAGGCACACAGTGCCAAACAAGACAGAA | |
| ATCCAGAAATTCATGGAGCTTAAATCATGTACTCTTTTCAGTTTTTAAAATTTTCTTCCTGGGCGTAGTCGCTCATATCTGTAATCCCA | |
| GTACTTCACCACCCCGAGAACCCACCATTGCTTAAACCCAGGAATTTCACACCAGCATCGACAAATAGCGAGACCCCAAGTCTCTACAA | |
| AAAATTTAAAAATTAGCTCCCCATGGTGCCACATGCCTGTGGTCCCACCTCTTCACCAGGCCGAGGTAGGAGGTTCACTTCAGCCTCCG | |
| AGGTTGAGGCTTCAGTGAGCAGCGATTGTGCCACTGCAGTCCAGTCTAGGCAACAAAGCAAGACTGTCTCAAAAAAGGAAAAAAAAAAT | |
| TAGTGATTTATCTCAAGGTCAACACCTTTAACCTAGTGGGTGGTCAAGAAATAGAACACTGGCAGCACTGCAAAAGCATCCTATCTGCC | |
| CTTTCTTACTTAGGACCCCTTCCCTCCACTGTCATTTAAAAACATTTCACAAACAACTTTTGGATTATATGTATGTCATTCTGTCACTC | |
| ATATATATCCACATAGACAATGGAGCGTTGACTGAATGAATTCATTATTCCAACGTCCTAAATAATGACCTTCACACCAGCGCGACTCT | |
| GCTGTGTCAGTTGGGCTCTAAGCTCAGTTCATTTCAGATATTCCTCACTAAACAAAATACTGTTTTTCCTCAGTTGTTAGTAAAAAATA | |
| GAAAACAGATTACATTCTCATTTACTTATTACTTATCAATGTAAAACTAATTATTTTGCTTAATACCAAGTAGCCACGCTCACTCACAG | |
| AGACATGGGACAAACAACTATTCACATCTCATGTTATCAGACATAGAAGAGAGAGGTATACTTCACCTATTTCTCCCCACTCAGTTTAT | |
| ACATTCACATCTGTATGTGTGTTGTTTTGTTTTGTTTTGTTTGTTTCTTTTGAGATGTGGTGTCGCTCGTCACACACGCTGCAAATCCA | |
| GTGGCCCAATCATGGCTCACTGCAGCCTCGACTTCCTGGTCCCAACCTATCCTCCCACCTCAGCCACCCAAGTAGGTGGGACTACACGC | |
| ATGCACCACCACACCTGGCAAAATTTTAATTTTATTTTACTTTTTAAAATTTTTGTAGAGATGGGGTCTCCCTATGTTGCCAAGGCTTG | |
| TCCCCAAATACCTCGACCTCCCAAAGTGCTGGGATTATAGCTCTGAGCCACCACACCCAGCCATGTATATGTTAAAGCCTTTATCTGTA | |
| TGAGTGGTCAGTGTAGAAACCGCTTCATGGAGCCTGATTTTGATGGCTTGCAAATTATTATATTTCTCTATGTCTAATCGGAAGGCCTT | |
| CCCCGTGATTATTTCTTCAAAGGAACCCTTTCTCCCTCCAGGGTGATTGTACCCATGTTCATAGGACCAAACATTCCTCGTTCACATAA | |
| TGACTCTTTTTCTTTTAAAACATAAAGCACATATTTTTATGGATTTTTGAGGATTAATAGAATAAAAATCTAAAGTCATATTCAGATTC | |
| AAGAGAACTGGATCAGTTTCTCTGTATTTCAACAAAAGAATACTAATCTCTAATTCATAATGCATGAGGTCTACTTCTTCCTCAGTCTG | |
| AAAATATGAATATTCCAAAATGGTACTCAAGGCAGAGGCTATATATCAAAATTAAAATTCTGTGATACTATGTTTTTAGGGAAGAAGAA | |
| AACTGTAGCATTAGAAATGTAGACGCAAAAGGTCAGAACAAAAGTCCAGTATGTCAAGTCCCTTTATAATGCCATCTGAATGAAGACCT | |
| CACACCATGAACAGAGGTATTGAAGCAGTTTTTATGTGGAAATGGTAGGCAGCAATATCTCAGGAGTGACTTTAAATTATACATTTAAG | |
| GTTTTATCTAACTTATTATAATCCTTAGAGAACTTCTCTAGCTCTACAAGTTATTCTTGGCAGAATTTGTCCTAACATAGTATTAATAA | |
| AAATAAATTTTACTTGAGTATGATATAAATTACTCTTAATTACAATATAATTTTAAAATGCCCAAAACATCTTTTGATCTTATGAATTT | |
| CAGTCAGCTATGATTAAGAAGACAACCATAGGCAAAAATTTCTTTCTTATGGAACACGTTGGAATGCATTCTAATCACTTCTCTAAGAA | |
| AATCCCTGTCCCCCATGATTATAGTCAATATGATACACCAATAATTTAGATGCTTCTTTCAGGGGGGTGACGTTATGTATTCTACAGTC | |
| TATTCATTCTAAGAGGCACTTTTTTCTCCCCCAATTTAACAGTTTTCTTTAATTATATACATTTCAGGATTTTTTGGGGATATTTTTCT | |
| ATCACTGGTTTCTGATTTAATTCCACTGTATTTAGATAACATATTCCGCATGCTTTCAATTCTTTGAAATTTATTGAGACTTACTTTAT | |
| GGCTCGCGACCTGGTCTATCTTCGTGAATATTCCGTGTGTATTTAAAAATAATGTGGACTCTGGGCCGGGCGCGGTGGCTCACGGCTGT | |
| AATCCTAGCACTTTGGGAGTCCGAGGCAGGCAAATCATGAGGTCAGCACATCCACACCATCCTCCCTCACACGGTGAAACCCCATCTCT | |
| ACTAAAAAAATACAAAAACTTAGCCAGGCGTTCTGGTGGGCGCCTGTAGTCCCAGCTACTCAGGAGGCTGAGCCACCAGAACAGCGTGA | |
| ACCCAGGAGGCAGAGCTTGCAGTGAGCCGAGATGGCGCCACTGCACTCCACCCTGGGTGACAGAGCGAGACTCCATCTCAAAAAAAAAA | |
| AGAAAATAATAATAATAATAATGTGGACTCTAAAATCATTCAGTGGGGTGTTGTAGACACACTAGCTTCTTTTGATTGACAGTTTTGTT | |
| CCTAAGCCTTCTATATCTTTCCTACCATCGTTTGAATGTGTCCCCACCAAATTCAGGTGTTGAAACAGGATGACCAATGTGATGGTAGC | |
| AAAAGGTACAGCCTGTAAGAAGTGATGGGATTAGGTGCCCTTTTAAACGGCTTGCTGGAGGAAAATTTGTCTCCTCTTGCCTTTCCGCC | |
| CTCTGCCAGTTGCGGACACAGCATTCCTCCCCTCTAGAGGATGCATCCCTCACCAGATACCAAATGCTGGTGCCTTGATCTTGGGCTTC | |
| CCGTTGCCAGAACTGTGAGAAATAAACCTGGGCTCTTTATAAATTACCCAGTCTCAGCTATTCTGTTATACCAGCATTAACACACTAAC | |
| ACATTTCCTGACACTCTGTTGATTTGCTTTGATCAATTACCGAGAGAGGAATGTTAAAAATCTTAACTGTAATTGTGGATTTGTCATTT | |
| TTTCCATTAGTTCTGCCACGTTTTTGCTTCATGTATTTTGAAGCTTTGTTAATAGGTGCGTACACAATAACATCATTGTTTTTCTTTGA | |
| TCAGTTGGCCCTTTGATTCTTATATGCAATGTTCCTCTTTGTCCCTGGTAATGTTTATTCTCTTGAAGTCTACTTTATCTGATATTTTA | |
| GAATGTAGCCTGCTTTTCTTATATTTAGCATTTGCATGATATAACTAGCCCACTQTACCATCTTCATACATTTAAACTGTGTCTTTAAT | |
| ATTAAAGTACAATTCTTATGAAAACCGTATACTTTAATCTTGTTTTTTAAAAATCCAGTCTGATCATCTCTGTCTTCTAGATGAGTTCA | |
| GTCCACTTCCATGTAATATTATTATTATTATTATTTTTTTTTTTTTTTTTTTTTGAGACGGAGTCTCGCTCTGTCGCCCAGGCTGGAGT | |
| GCAGTGCCGGGATCTCCGCTCACTGCAAGCTCCGCCTCCCGGGTTCACGCCATTCTCCTCCCTCAGCCTCCCAAGTAGCTGGGACTACA | |
| GCCGCCCGCCACTACGCCCGGCTAATTTTTTGTATTTTTACTAGAGACCCGGTTTCACCGTTTTAGCCGGGATGGTCTCGATCTCCTCA | |
| CCTCGTGATCCCCCCGCCTCGGCCTCCCAAAGTGCTGGGATTACAGGCCTGAGCCACCGCGCCCGCCCCCATGTAATATTATTACTGGT | |
| ATGTTCGGTTTAAGTCTCTCATCTTGGTAATTAATTATCTATTTGTGCTATCTCTTCTTTATTCTTTTTTTTCTCCTTTCCTGCCATCC | |
| TTTGAATAAATCAAGTTTAGTGTTTTAAATTCCATTTTATCTCCTTCATTGGTTACTTTCTAAAGTGGTTACTTTACCACTTTAGAAAA | |
| GTATTTGTTTATTACAATATGCATCTTTGAGTCTCTAATTAATGTGAGAACTTGACAATCTTATACTTCTGTTTGTCCTTCTGTGCTGT | |
| TATTGTTATACATTTTACTTCTTTATATGTTATAAACTCCAAATATATTCTCTTGTTTTGCTGTGGAACACTCAATCGTCTGTTTACCA | |
| AATTAAGAAAGAAGAAATAAAAGTCTTCTGCTTACTCACATATTTATCATTATTGATGCTTTTTATTCTTTCATACAGGTCCAAGTGTC | |
| TATCATTTCCCTTCAGCATGAAGAACTTCTTTTTAGCATGTCTTTTAGCAGGTCTGCTGGTGATGAATACTCTTAGCTCTTGTTTACTT | |
| GAAAATGTCAATTTCACCTTTATTTTTAAAAGATTTATTTGCTCAATATTCTGGACCAATCTTTTTTTTTCTTTTATCACTGTAAAGGT | |
| GCCTTTACATTGCCTTCTAGCTTCTACTGTATCTAATGATCATCAGACATCATGCTTATCATGCTTATCAAATTGTTCCCCCATAATAT | |
| AATATGTCCTTTCTTGTCCTTTGCTATTTTGGGATTTTTTTTTTCTCTAGCATTAGTTTTTAACAGTTTGGCTGTAATATAACCTAAAA | |
| TGGTTTTATTTGTCTTTATCCAGCACAGAGATCTTTGAGATTCTTGGATCTGTGGGTTGATTTTTTAATCAAATTTATAAAAAAAATTC | |
| TCAACTGTTATTTTTTTGAATATTTTTTCTGTCTTTTTTCTGTTTCTGGGATTCTATGTTAGLAATACGTAAGACTGCTTAATATTGTC | |
| CTGTACGTCACTGAAGCTGTGTTCACTTCTTTCTGCCTTTTTTTCTCTGTAATTCAGTTTGGGTAGTTTTTATTGACCTTTCTTCTAGT | |
| TTATTGGTACTTTCTTCTATAATGTTAAATATGCTAAGTCCATCTACTGAAATTTCATTTCAGTTCTAAAATTTCCATTCTTTTTTTTA | |
| AAAGAAAGGTATGCATTTCTTTGCTCATATTGCCTATCTGTTCTCTCATTATGTTCATCTTTTCCTTTACATTATTGACCATATGCTAT | |
| TAGCTATTTTTGTCATTTGTGTCATTTTTTTTCTCAGTCTTATTTTCCTATTTCTTTGCAGGCTTCATTATGTTTTATTGTACATTGTG | |
| GAGAGTTTGCGTTTTTTGTCTTCCTTTAAAACGTGTTGAGCTTTGTTGTAACAGGCAGTGAATTTACTGGTGGATCACCTAGTTTTATT | |
| AAGATAGGTTTAGAGTAGCCCTTATTTTAGGATGTTTCTAACTCCCATCGTGTGGTCTTTCTGAAATCACAACTGAATGCTCACAATGT | |
| TCACTGATGTCTTTCCACCCTGGATTATTACAAATTCAGTGTCTCTCAGCACTATATGATGTCTGGAATTTCCATTTAGCTCTTACATC | |
| CCCAGAAACTGTTTTCTATTAGGTTTTGCAGGGTCTTGTCCTACACTATCACAGCTTGGTATTTGCCCAGTGACCAGAGATGACCCCTA | |
| TCTAGATTTCTGAGGCTCTTGCTTTTTGTGTAAAGCCTTTCTTTGTGATATTCTGCCCATTGGTGTTCTCTCTTTGGCCTCTCCCTGTG | |
| TCATGATTTGGAAAGTGTCCTCAGTCAAAAAGTGAGCCCGAATTTAGAATTTACCTAAATCACCTTATAATATATAGCCCTATCAAGTC | |
| CAATGTCTTAAATTACATCTTTAGGTATTTTCTATGGTTTCACAGTTGACTATAGCAAGGACCCACGTTCAGATAACTGTTGGTCTGTG | |
| ATAACTAAAACCAGAATTTGACATTCTGACAAACTTGAAATTGCAGTGCTTCTTAAAATTAATACAGTATTACTTAAAAAACAAAAACA | |
| AAAAAACCAGTGTTTTCCAATTTGCCAATACAAAAAAGCAAAACTCCCATCTCTACTTTTTAGGAACTTCTATCCATTTGAGAAGATGA | |
| TAAATATATAGGTAAAGTTAGATTGCAGTAAAACTATGCTGTCTAGTGTTAAATCACCAGTATATATAAGTTTTATAGATATTATGTGA | |
| CTACACACCTATAGCTTATAGGAAAAATATACCATTTCTTGGCAGAAATTCCATTTAACTACAGCCTTGAAGAGTGGATGCTATTGAGG | |
| TAGAAAAGATGGCATTTCAGGATGCAGGGCATATTGTGAACAAATGTACAAGATGGAAATGAGCAAGTCATTCTGAAATGTTAATCTGT | |
| CTATCATAGAGGGTTTGATTAGGACATAGTAGTAGTTGATGCTTATAATATGATTTAATATAATAATGTAAATGTGCTGAGTGGTTGAA | |
| GTCTTTGGACTTTATCTTATAAGTAATATGGCAATAATGTGAAAGATTCTTGAAGAGGAAGAAACCCTGCAGTGAGGGAGAACAGTACA | |
| GAGCCTATTACTGTCATTCAGAGGTGAAGCAATAAAGGTCTCTCCTAGGATGTTGGAAAGAAGGGGCAGATCCAAGAAACAACATGAAC | |
| AAAATAATCAGCAGAAATTGGTTATTGACTAGATATGTGAAAATGATATATTGGCAGAAATTGCAAAGGGTCAAAAATGGGGCTCATTG | |
| CAGCAGGAATGATGAAATTGCTTTTAGATAGACACGTGAAGGGATTTAAACATTAAAACTTCTTTTAAGCTATCACTGCTTAAAGCTAA | |
| CAGTCATTTATTAATATTTCCACTCTCAAACTTTTAACAGTTTTAATTTTATGTATACACCACAGTGTTTTCGCTACTATACTTGCCTC | |
| TTTTGTTCAGTTGGGAATTGAGTAAACTAATAGAGTAAAAAAATGAGTACAAAGTGGGCTTTGACTAGAACTCTAACAAAGTGTGATCA | |
| CTGAATTACCAAGATAATTAACCCATACCCCACATATACTCCATTTCCCATCACACATATTATATTGCATTGTTTTTGCCTATTTCCCT | |
| TCCTACTTCCTAAACTGTTGAATTAGTTTGCTTTTGTTGCATTAAAAAAAAGGAGAATCACCACAAAGCAAATTAAAAGATACTGATTA | |
| CTTCTTCCAATTCTGTGCCCTGCCTAGTTCATCTCCCCTGCCCTGCTTTGGCTAATCTCTGAGGTCTTTTCGCTACTTGACTGGTACTG | |
| TTTGTAGCTACTCAGCTCCTGCCCTACTGCTGTTTGTCTGAAGTTGTTTTGGCTACTCAGCTGGTGCTACGCAGCCTCAGCTAGGACTG | |
| CCTGTCTGTGCTCCACATGGTCTTTGATTTTCCAGTGGCTGGTGCAGGCTTGTTTGCATGGTAACAATGTTCCAAGAAAGCAAGAATGA | |
| TCATTGCAAGGCCTCTTATAACCTGGGTTTAGAACTTGTACTTCTACCTCTTCCTATTGGTCAGACTATGTCCCTAGGTCTGCCAAGAC | |
| TCAAAGCACAGAGAAATAGATGTTTCCTCTTGTTGAGTGGCATATAAAAAAAAGTGTGGCCATTGATTTCAATCTGCCACAACCGTAAG | |
| CTCTGTGACTACAGGAACTGCATCACTCTTTTCCACTTTGGTGTTGTTGGCATACTACACATAAATATTTTTAGGTCAATAAACTAATT | |
| AATGAACGAAGGAAAACATTCAGGTAGTATTTCAATATACTGTAGTTTTAAATTATATATATTATATACATTTGACATAGTTTAATATA | |
| TAAGTATATACACATATAGTTTTCATATATAAGTATATTATATATTATATAAAATATTTATATGTTATATATAAAGTATATATATAAAA | |
| TATATAGTTTTAATATGTAACTATATGTATATAATTATATATAATATTATAAGTATATATACTTTTATCATACCATCAGTGAGTATAGA | |
| CATGGTTCAGGGAGAATCTCTTTATTCTCCTTCTATCTTTTGAGATAGACCAAAGAAAATTTTTTTTTAAAAAAGGAACTATCACTTGA | |
| GTTAACATTGTTTGGTATTTTTATCTTCATAGGTGATTGCCTTGTGAATAACTCATTTTGTGTATTGTCAGAATCCTATTCTGCTTTTC | |
| AGGCTGTGCTATGGGTAGACTAGCTCTAACCAGAACACTTGGGAGACCTGAAGCAAAGATACTATATTAAAATAAGTAATGTTATATGC | |
| CAAAGAGGAAAACCACTTCTGCCTCCTAACACTGAAGAACCAGAGACTGTQGGGTCACATTAGCTAGCATTACTATATGTAAAACCTGA | |
| GCAAATGACTTACTCTCTTTCAGCTTCAGTGTCTTATCTAGGACATTGCTTCTCAGGATTGGTGTGAGAATTAAACAGAATATGTGAAG | |
| TTCTAGCTACAGTTCTTTACCCATGACGTAGTCAGTAAGTTTACTTCTTTCTCCATGTGAACCAAAGCACTGTCATGATGTGAATGATG | |
| TTCATTAGGGTTTCTCATGCTCACCGTTATTGATATTTACCGTTGCATATGCTTTATTGTGAGGGGCCATTCTGTGCAAGTATAGGATG | |
| TGTAGCAGCATCTGTAGCCTCTATACACTAGATGCCAGTAGGACCACCTATCCAGCTGTGACAGGCAAAAATGTCTTCAAACGTTGCCA | |
| AATGACCCCTCCGGGCAAATGCAAACAGTTTATCTAGCAGTGACACATAGCCAGCCACCTTACAAGGGAATTGTCAAAGCGTACCCATA | |
| AGTGTAGAATGGGACALAATACTTGAGTCTTCTCAGAATGTCTAAAACCTTTTGCTGTCTATAATTCCTCTTTTTATAATTTACCAGAA | |
| ACATATATTGCTTATTATGCCATCTAATAAAATGGCTAACATTTGCTGTGTATTATACTCGGTGTTAATAAGCTTTGTATGAACTCACT | |
| AGCTGAATGTTGTATATGTATCTGCCATACCTGTTGGATTGACATTCCACTGTAAACAGGTACCTTATCTTTACCAGCTCCAAGCACAA | |
| TGTATTTGGTATATCAGGTAGCTATTCTACATGTTAATTGATCACATTGCACCCAAATGAAACATGGATGTGGAAGTGGAAGGAGGAGC | |
| TTTAAAACATGGAAAATTAGTTTATACAAATCTAAATGTTGTAGTGCACCAAATTGATTCCTTTTTAAAAACTTTCAGAAAATCAGCTT | |
| TAAAAAAAAAAAGTAACTAAGCCATGATCAACTTTTCTTTCATCACCACTAAGCCAATATCAGAAAGTTGTCAATCAACAGCTCTTTTA | |
| AAGTTGAAATTTTTCTATTGTATTTCTTATCAAGATTTCAGTAGTGCTTAGAAGTTTTTGGCTCATTTATCATGTTTTATTCACTGTAT | |
| GTCATTAATTGTTAAAGATGTCAGAGTCAAACCTTAAATTCAGGAGAGATTTAAATTTTTCCTTTTTCCTATTTAAATATATGTTTCTA | |
| ATTACATAATTCAGTTAGAAAATTACAATATAGAAACATATAGACTTAGTAAAGTTTCCAGTTACTGTATACAGATTATATTGTATACT | |
| GTATTATTTTTAACAGTTGCATATTTAACTTATAAATGTGGATAATTATAATTAAACAATCTGTTATCAAAGGACATACAGATTATTTC | |
| CAGTTCCTTGCTGCAGTGAATATCCTTGTACTTATTTTTTTACACACTTGCTCAGATAATTTTTAAGTACAAATTCCTGAGGTAGAATT | |
| GCAGAATCAAAAGATACTCTCATTTGAGTGTTTCACATTTTATGCAAAGTATTTTGTTTCCTCCAGAAAATTCCTCTGTGGAAGAGGCT | |
| TCCTCACATTCTTTATCAGTTTATTAGCCAGCAGAGAAATGATGAAAAGCCACCCCTTTTCTTGTAGTTTCATCCTCATTGCTATCCAA | |
| GATGAGGTATTAAAATCACACCTAAGTGGTGTCTGTTGTGGCTGAATATAATCTTGAAAGTAGAGACACTTCTCTCTGATTCCCAACTG | |
| TACTCTCTGGATGGCACCTAAAATTTTATTTTTTCAAACTGAAAATCATTTTAATGAGTTCGAAAAGGTTTTATTTTTTTAAAATGAAT | |
| CCTATAGAAAGAATACATTTTCTTACAGTGGGTCATGGTCALAAGTGTGAATATCTCTGATCCTACTTAGTTTCCCCAGTTACAAGGAC | |
| AGGCGTGCCCCCAACTCCATCCTTAATGGCTCAATTCTGTGGCTTTCAAGTTAATCCACTCAGTCCTACAGCCTTCACCAAGAAGCTCA | |
| TGTGCTACACTTTGGTGTTAGTGGGGGCATTGTCACACAGGTTCAGAAAATAACCCATTAGGCATTTTATAGAACTCATCACATGTTCA | |
| AACTAAGCTAGTTGTAACATTATGTTTTTCATGTGAAAAAACACAAGCAATTCACTGGCTATTTTATTTGTTGCTAGCTATGCTATACT | |
| TTCTATTTCTTTTTCCATGGTGTGATCTTCCTAGAAAACCTCACTACTGGGAAAAGAGATGCAGAGATTAGTCCCATCTAATCTCCCCT | |
| GCAGAATCAAAAGATACTCTCATTTGAGTGTTTCACATTTTATGCAAAGTATTTTGTTTCCTCCAGAAAATTCCTCTGTGGAAGAGGCT | |
| TACTCTCTGGATGGCACCTAAAATTTTATTTTTTCAAACTGAAAATCATTTTAATGAGTTCGAAAAGGTTTTATTTTTTTAAAATGAAT | |
| TGCTTTTATAATTAATGCAAATATTACTTAGTGATTCATCCTTTCCAATGATTCTTGGTTTCTTTTGACATTTCATTTTTTAAATTGTG | |
| ATTAACATTGACATCACCATAGCGTTACTGTTACCTCACCTCTTTACAGTGAACTGTATTGCAAAGCCTATAAAAGTTCAAATTTTTAA | |
| CTATCACCCACCTGAGCCTTGGACTCCTGTTAGGCTTTTGAACTCTGATCCTTTGGCAAACATTGGTGTATAATCATTTCCCCACCATA | |
| GAAACGGTTAATCTTTTCAAGGGAAGTGAAGCAACTCGGAAGGCCATAGGAATCCACATCACCTGAACTGCAGACCAAGCCTTGGAACT | |
| TTGTTCTGTGCTCCACATGGTCTTTGATTTTCCAGTGGCTGGTGCAGGCTTGTTTGCATGGTAACAATGTTCCAAGAAAGCAAGAATGA | |
| AAAACTGACTTACTCTCTTTCAGCTTCAGTGTCTTATCTAGGACATTGCTTCTCAGGATTGGTGTGAGAATTAAACAGAATATGTGAAG | |
| ATTTTCACCCACCTGAGCCTTGGACTCCTGTTAGGCTTTTGAACTCTGATCCTTTGGCAAACATTGGTGTATAATCATTTCCCCACCAT | |
| TGTGGTATTCTAGGATAGCCATTTTGACATTGATTGTTCTAGCTGGTATTCACTACTGTATTTAAAGTGTAATCTTTTTACTAGTATTC | |
| TTCTCTGACTTACTCTCTTTCAGCTTCAGTGTCTTATCTAGGACATTGCTTCTCAGGATTGGTGTGAGAATTAAACAGAATATGTGAAG | |
| TAATCCTTGCTGCAGTGAATATCCTTGTACTTATTTTTTTACACACTTGCTCAGATAATTTTTAAGTAACAAATTCCTGAGGTAGAATT | |
| CCATTCCATCAAAATCTGTCATTTTTACAGATGAAGAACAGCCCACAGTGAGTTTAGTTAATATCCTAAAAGATCACACAGCAAGCATG | |
| ATAGAACTCTTAAATTTCAAGCGACTTACCCTCTTTACTCTATCAGGTCAAATTTCCCTTTTTTTAACTGCTTGTCTTAATTGAGATTT | |
| TTTAATTGTGGCTGTCTCTATATCTTCAATCAATACTTTCTTGTTTTAGACATGTTTCCCTCCAAATCTGCATGGAAACTTCATGTTCT | |
| GAAGGACTCAGAGCTGCCCTTTCCTTTTTGTTAAAGTTGTACACTGCTCTGCATCCCCCTAGAATGCTCATCAGCTTTCTCACTCTTCC | |
| TCACTCACACAGTGCTCCAGCAGGAGAGGATTTCTTTGAAAATCAGATTCTTTTTTGGAGGGCTTTGTGCTTATTGGAGTGTTATGTGA | |
| TATATTCATTACGGTGAGGGCTAAACTCTTTGAACAAAGAGACCTGACAGTGCATTTGGGTTAAAGAACAAAGTTTATTTCTTTCTCTC | |
| ATCACCACCACCATGAGCAGCCTCGATGCTCAGAGCAGATCTGTCTCATGGCATTGTTCACCGATCCTCATTCCTTCCCTCTCATTTAT | |
| CTGCCATCCCCTACGCTATTAATCATCATGTTTTTCGTACAAAGCCAGGTCTTCTCCATGTCTGAGTTTTTATGGAGG1AAGCATGTCA | |
| GGCCTTAAGCCCGTCCTTGCTGCAGTGAATATCCTTGTACTTATTTTTTTACACACTTGCTCAGATAATTTTTAAGTACAAATTCCTGA | |
| GAAGTGTAGGGTATACACTAGGTAAGCAGTCACATTTCCAGCTACAATACTAATTCTATGGAACGAGACAAAACATATTTTGATCGCTA | |
| CCTTTATTTGAATAATAATAACCACATTTCTCAAGGAAGTAAATCTCAGAAGATATCTGATTTTAGATACTATCTTTATCTGACCATGT | |
| GGTTCCTTCCTTCTATCACCGAGGGCCTCTCGGTAATAGACATTGCTAGCGCCTCCACTATATTTTGTTTAGCTGGAGTTGATTAGAAA | |
| TCTGAGACCTATAGCCTTGATAATAAGCACTTTTATTTTTGTATTTTTATTGCACTTTTAATATTACACAGTTTCTCTACCACCTTGAC | |
| AACACCAGAAACATGGCTTCCTTCTGTGGAACACGCTAGAGCACCAGTCCCCAACCTTTTTGGCATCACCCACCAGTTTCGTGGAACAC | |
| ACCCTTTCCACACACTCGGGGCTGGGGGTGATTTTGTGATCATTCAGGCACATTACATATATTCTGCACTTTATTTCTATTATTATTAA | |
| CATTGTAATATGTAATTAAATAATTATACAACTCACCATAATGTAGAATCAGTAGGAGACCTGACCTCCTTTTCCCACGACTACACAGT | |
| CCCATTCCTTGCTGCAGTGAATATCCTTGTACTTATTTTTTTACACACTTGCTCAGATAATTTTTAAGTACAAATTCCTGAGGTAAATT | |
| TAGATCCCTTGCATGCACAGTTCACAATAGGGTTTATGCTCCTATGAGAATCTAATGCCACTGCTGATCTGACAGGACACGGAGCTCAG | |
| GCAGTAATGCAAGCGATGGGCAGCACCTGTATATACACATGAAGCTTGGCTGGCTTGCCTACTGCTCACCTCTTTTTGTGCTGCGTTAT | |
| CCATACCCCAGGGTTTGGGGACGCCTGCACTAGAGGACAGAGCCTGCTACACTATAGCAGTCCTCCCCTTGTGTTATGCACAGTACTGA | |
| TCAGTCCCTTTTGAGCTTCTCAACCTTGTATAAAACTTTGAGACAGTTCTGGGACCATCACAAGGTTTATGAAGGGTTAGAAAACAATA | |
| CACTTAAGCAACTAAGACTGTAAATCGTTGAACTTCACAGGGCGATAAAAAGAAATGGCTTCTAAGAATCCCAGTATGTATGATTTTTT | |
| ATACAAACCATAAAGATGATTTCTACCAAACTCAGGACACTGTGGCGAGCTTAAAACACGAGGGTATTGAATCATACGTATGGTAATTC | |
| CTAGATTAGGTCCTTGCTGCAGTGAATATCCTTGTACTTATTTTTTTACACACTTGCTCAGATAATTTTTAAGTACCTGAGGTAGAATT | |
| TATTTGGGGGGCTTAGCCATTCCTTGCTGCAGTGAATATCCTTGTACTTATTTTTTTACACACTTGCTCAGATAATTTTTGGATGCCAT | |
| GTTTTGTTTTGTTTTAATACACACCTTCAGAATCGCAGAAGATGATCCATATTTGGGAAGGCCTCAACAAGTAAGTGTCATAATCTCAC | |
| TGATAACTTTATTTAAATATATTCATATTTCATATGCAAGGCAACATTAGTAAGTTAGCATTAGGTAAATGTATATTTCTAATTAGTAA | |
| ATTTTTTTTTTTTTTTTTAATTTCACGTGCGTCCGAGAGTTCCCCCCAAAGATATCCGAGGGATAATATATATTATATATATATGGGGC | |
| ACATATTGTTGCACAAAAATTGTATATGCTACAGGATAGCTATTGTATTAGCTTTCTAGAGCTACCATAACAGATCACCACAAACCTGA | |
| TGGCTTAAAACAACAAAATACATTATTTCACAGTTCTGGGGGCTAGAAGTATCAAATCAAGGTCTTCCCAGGGTTGGTTCCTTCTCGAG | |
| GCTCTCAGGAAGAATCTGTTCCATGCCTCTCCCTAGCTTCTGGTTGGTTGCTGGGAATCCTTGGCTTGTAGCTTTATCAGCTCCAATCT | |
| CTTCCTCCATTGTCACGTGCGCTTCTTCCCGGCGTGTCTCTACTGTGTCTCTAACCTAAATCTCCTCTTTTTTTCTTCCAAAAGGCATC | |
| ATTGGATTTTAGGGAGTCTATATATCCCATCCAGAGGATATATACGCAGCTGTGCCACGTCAATCCTAATATATAGTATATATATATTA | |
| CACAGGTACCGGGGATTAGCAGTTAAAGGTGTCTTTTTGGGAGCGAACACAGTTTAACCCACTACAGCCCCTTGACATATTTATTTAAT | |
| GTGTACACATGGATGTAGAGAGTGGAATGACAGACCATGGAGATTCAGAAAGGTGAGGGATGGGAGGGCAGTGGGATGGGGGTGGATGA | |
| TAAGAAGTTACTTAATGGGTACAATCTATGTTATTTGAGTGTTGGATACCCTAAAAACCCTGTTTTCACTACTATACAATCTATGCATG | |
| TAACAAAAATTGCAGGGTTTATATATGGGTCCCCGGTGTATATACCGGCATGCCCCAAACTTTGCGAGAGTGTCCCCCCCCAAAGAGAG | |
| AAAAGATCTTTGAACTCAGAATTTGAAGATCTTTATATAAGTACTGACTACTACAGATCAGCTCCTCAGACTTGAAGAATCACTTAGTC | |
| CCCGTAATCCCTCACTTTTTCTCATTTGTAAATAGAAATGCTAGCACCACACTAACAGGGATGAGTCAAAAATATTCAATGAATGTGCT | |
| TGTAAGCATAAAACTGTTCCTATCATTATATTAGTAGTTATGAAAGCATACCAGTGTCCTTTTTTCTTTTTCTCCTATATTTTATTTTG | |
| AAAAATGTTGGAAAAAATAAATAGAAATGTTGGAATAATCTAACAAATCTACATTGGTTCTTAAAACCGACATTTTCGCTTTTCTCTTT | |
| GCTCTCTTTTTCCTGTGTGTGTGTGTGTGTATATATGTATATATGTATATGTATATACATAATCATTTCGAAGCAATTTTCAGACTCTG | |
| GAGCAATTATAATGGCAATATTAAATTATTTATGTAATGATTCCAAGAAGTATTTAAAACATGGAGATTTGTTCATTCATCAGCTATTT | |
| ATTGAGGTATTACTACGTGCCAGAACATTGGCTTAGTCGGGAAAAAATAAGTGATATATACACTCTAATTTCAGGACCCTACTATTTTA | |
| ATAGGAAGAACAGAGACATTGTTTTTCATATGGTGAATATGTATATCCGATAGGCCCTATGCATATATTTTCCGGGGAAAGAGACAGAG | |
| ATCACTAGGTGCTACCTTGGAGGAAGAGCAGAGTTACGGAGGACTTTCCCAACCAAACTCTTCGTAACCTGTGTTTTGAAGATCTAGTT | |
| TCCTAGACTAGAAAAGGTGAAGGAACACTAGCTCCCAGGATAGCCTGGTCACATTTAAATTTTAGTGGCAATCATTTAAAATAGTGGAT | |
| TTCTGAAATTACACGTGCAAAGAATCAGCCAGAATAACAGAAGTGGAATATGTTTTAAAGGCTCTGGTATCATTTCTATTAAAAAAAAT | |
| AAAATGGACATATATAGCATTTATCACTTCGGGTCATATTCTGATTGATTTAGAAAGAATGACTGCATTCCTTTTCACATTAAGATGCT | |
| CCTTTCTAATTTTATTTTTAAAATGGTGATTTACGATAAATCTAGGAACATTTTCTTTTATTCCACCCAAAACATTTCACTAATTCTAT | |
| TATCAAAACCGTTTGTTTTACCTTTCGCTTTTAATGTGCTTTCTCTAACAATTAAGGGCGAACTAACCAGCATGAGGATTGTGTCTGCT | |
| TGATTTTAAACCATCCTTTCCTGTCTGTACACAGGAAATCTTATCAACAAGAGATGATTCTTTATGCCACAGAAGAGTAAAACCTGCTG | |
| AAAACATTCATTTTGAAAGTTTTTCTTATGTGGGCATCTTTGCCATCATCAGTAACAGGGGTGGGGCATATTCTGACATTTCCATTTTT | |
| GAGGCAAGCCGTCTTGAAGTTTTTTTCTTTCTTGGCTTGAGCTTGGTGGTTTATATTTCCTAAAGTTTGCATGTCCTCAGTGTTTTAGT | |
| GGACTCTGGAATTTGCTCTCCACCCTGACCTAATAACAATGCAAGCTGCAGCACAACACAACTTCTCTCTTAGTAATGAAGTGAAGAGT | |
| ATATCTCTCTCCTAAGGTGAGCCCTTTTGCCCACTCGCCAGTCATTTACCCACCACAGGAGAGTTTTCCTGCTAGGTTATAAACAATTT | |
| GCTCTGAGACTGTTAAAATCCTGTATCAAATGAGGCCCATTTTTGCAGAACATGGTTTTTCATTCCTGTATTTCCTTTTCAGCAACAAT | |
| TGAATACCATCAGTGAGCTTTTAACTTATAAGCATTGAAGTGAACCGCATTCTTTGTTCCTCAGTTGAAAATTTCCATCCCTCAAAATC | |
| GATCTGAAGTACTGTTTATTTAAACGAGATTTTTCTTACTCTTAATTTACTAGCCATACCAGGATCTTCAGTACCCCAGGAACTTTCAA | |
| TAATTTACTACTAAGGGCAGGAGACAAACTGTAATACTCGCTACTAAAAGAAACGAAAAGCTAGTTCAGCACACTTACTAGTCTCTCAT | |
| AAAAAGTAAAAATATGGCTGGTTTCCATGCTGTCCACAGACATTTCAAACATATAACTTCTTTAGGCTAAAATAAATAGCAAGCTCAGA | |
| ATACAGTTTTAAAGTCCTAAATGAGCCAGCGGTTACAATGTTTTTTGATAAGTAATGAATAGTAATTTATTAAGATAAAAATGTACTTC | |
| TTCAAAATATTCTGCATGCCCACTCTGTTGCTGCCCTAACACATTACCACATACTTGGGGCTTAAAACAAAACACAATTATACTTCTCA | |
| GTCAGGAGCTCAGAAATCTAGAATCAAGGTGTTGGCAGGGCTGCTTTCCTTCTTGCGGCTCTCATCTCTTTGCCTTTTGAGCTTCTAGT | |
| GGCTGCCTGCTTTCCCTGTGCACCCTCTCATCACTCCAGCCTCTTGCTTCCATTGTCATACTTCCTACTATTCAGTGTGATTTCCTGAA | |
| CCCCTCTCATCACGCCCACCTGGACAATTCAGACACTCTCTTCATCTTATTATCATTACCTTAATGACATACGTAAAATTTCCTTTTGT | |
| CATATAAGGTAGCACTCACAGTTTCTCAGGATTAGGATGCAGACATGTTTAGGGGGTCCTTATTCAGCTTAACACACCCACTAAATTCC | |
| AGGTATTTTGTGTTATGCTAGGAAACTGAAGGTAAAAGTAATGAGTGAGCTCAGTGCTTAGTTGTCTACTCCTTGTACTCTCAGTTATA | |
| TTTTCAGTTATTCTGTAAATAGTCCCAGAGGTGCTCTCTAGCATTCTTTCAGGCCTTGACATTGGATAGCTACATGAGCCGTATATTTA | |
| CCACCATAGAGGAAATACTGATTTCTAGGATTATATTATTTTGTTTATACTTCTAAGAGAGTACAAGCTTCAAATCATTCATTCAACAA | |
| CTATTTAAGTATCTGTTCTGTGAATACCATGTACTTGGCCCTGAAGATATACCAGTAAGCAAGCTACACCAGGTCCCTGCCTCTGGGGA | |
| CTTTATACCCAAGTTATGCTTCTCAAATCCCTTTGTTACGTAAACGTTGTCCAACCCTATGTAAAGTTTTGTACACATGAGAACTACCT | |
| AGGTCTCATGAAGATCTTTTAAAACCCTGATTTAAAATATTTTCAGATTTTTCTTTTTGGCTTTAGTTTCATTCCTATTATTACATTTT | |
| ATCCTTCTTTGACTCATACAATATTTAATCCAGAAGTATTTCAACCACAGATGGCACTGCCAACAGGTAAGAAAACTCATCCCTGTTAC | |
| AGGTGTCCCAACTTCAAATTTTTTCATATAATATAGGAAAAATAATGATTGAAACATTTAAATGTTCTTCTTTACAGATGTTTCATTGG | |
| TGATTTCTTTTAAAAACCAATCTTTTAACATACTTTCTTGAATATATTTCCAAAAAATATTTCATCTCAATTACCCCTTCTATCCTCTT | |
| TACTGCATTTCATATCTGCTACGACTACTCAGAAGGAATTTCTTTAGTCAACATAACCATTTTCTTTATTATCTCTGTATTCCTTTTAA | |
| GTCCTTTCTCCCAGATTCACACGAGGCAGTCTCCACTCTTCATTTCTGTTTCAAACATTTAAGAGGGCCTGTTGTGTACTACCAGTGTG | |
| CCAGTCACTGGGGAATCAAAGTGTACAGAAATCATAAAAATCATCTCATTAGAAAAATCATCTTTATAAAAGCTTATAACTTTGCACTA | |
| TGACAAGTTGGATAAAACTCATAATCCTTCCTTCTACCATGACTGTATTAATTGTGCCGATTGGCAACTGTTTAATTTCGTAAGAGTAA | |
| TAAAAAAAAAACTCTCTTTCTTAAAAAAACTGATTTATTTGTATCTAAACTTGGTGTCATGCCTCTTTTTATGTTCTGTTTCTCTCCTC | |
| ACTTCACCCGTCACCAGTGGCACTTAACACATGTTCACCAGTGTGAGTGAATTATGCAAACTTATCTGTAAACAGAGTTTGCTAAAAAT | |
| CGCTGAAGGAGGCATCACCTCTTTTATTTACCAGGGACTTCCTTCTCTTCATTCCGGGACTTTATCCCATTTGTATTATTCTCATTTAA | |
| AATTATCATCCTTACTACAGTACTTACTTTTCTTTTCTTTTAGATCAGAAAACTTTTTCTGCATTTATATCTTTAGTATTTCTAGGAGT | |
| TCATGGGACCCAAAAAAGTACATCTACTATACTTTCACCTTCACTAGTAAGGTGTATTTCAGACGTTTTTCTGTCTTTACCCCTACTAA | |
| AACATTATTTTTACGCTGCCATGTCTGAGATCTTCTGAGTGCTGTCCAATTTCAGCTCTAGGTCAGCTTCCTGTGAGCAAGTTAAATGT | |
| GTTGGTACTGTGTGTGTTTCTTCGGAACTTTTTAAATTCTCGAGTTCAGTATTTTTCTTAGTATTAATGTATTACTCGAAATATTAAGA | |
| ACTTGGTTCTGTCCACAGGAAATACACACATTTTCACTTAATGTCTTGTGAATACAAAACAGTATCCTGAGGCAGACGTTTTCTTAATT | |
| TTATTCCTTCGGATTTATGGGCATCAAATAAGATTATCAAGATAACCATCTCAGTAAAATATTCTGTGGTCACAGCTTTATCTTGTAGA | |
| AATTATTTTCCCATAATATTACAAAAACTATTCAAATTACACCTAAGAATCTAATTTGTTAGTTGTTGTTACACCATCATTTTTACCTG | |
| TATGTTTTTCCCTCTTCATTCCAGTGTCATAGTACAATTTTTATAGTCCAAAGATATATAGGATGTTGTATTCAAATTTTTAAGAGGTA | |
| GAGAGGTTGATTATCTAAACCAATATTAACTTGTAACTCTAAAATGGATTATTTTAGTCTTCTAGATTTTTTGAAATCTAAAACCTTTA | |
| GTAGATCTAGTACTTCTTCAAGACTATGGGATGCGACATAACTCTTACTCTAAAGTTAAAATTTCTCTCCACACTCGAATGTCCTTTTC | |
| ATGGCACAGAAATGTGGCCAAAAGAATGTTGGCCTTAAAACAACAAATACATGCATACAAATCTAATTTTCTAGTTGGATTTAACTTTA | |
| ATGTGTTGCCTGAAAGAGGAAAAATGGCTCTATTTTACATGAATTTTCACATATTTAAAGGAAATTAAATCCTACAGGAAAAATAATAC | |
| CAAATTTCCAGTTGTTTTTAGCAGAGTTAATAAGTGAGACACTTCAACTTTAAATCTAAAACATGCTTCCCAACTCCTCTCTATTTTTC | |
| ATGACATCCAAATATTGCTCTGTTAGAGGACTGTCTTGATATGTTCTTTCACCATTTTAACACGGAATGTTTTTATAAGTGTTACATTG | |
| TCATACAGGCTGTATCTTAGTGTCAAAATATTTCTAATTTCTATCAAAATTTTATTTCCATATCCAATTAAATTCTAGCCATTTGGTGG | |
| GGGAATTTTTAAATTATGATTATGTTTCTAAAAGTAATTTAAAATGTCCAGGGGTGAATTTCATACAGACAAAAGATGGTAATTTAATA | |
| CATTGACCAGTGACTCCCCCTTTTTTTTCTTGTGTATGCTTCATTTCAGATGCATTCATTTTTCAAACTCGCTTCCTTGTAATTTCTTG | |
| AATAATACAGGAAGTAATTATACCCTTGTTTTCTCCAGTTCTCTAGCCTTATCCTGATTTATCATAATATTGAAGATTAAAAAAATAAA | |
| TTCTCTCGCCGCGTGAGGTGGCTCACGCCTGTAATCCCAGCACTTTGGGAGGCCAAGCCAGGTAGATCACGAGCTCAGGAGTTCAAGAC | |
| CACCCTGGCCAAGATGGTGAAACCCCGTCTCTACTAAAATACAAAAAAATTAGCTGGGCATGGTGGTAGGCCCACTGTAATTCCAGCTC | |
| CTCTGCAGGCTGACGCAGAGAATTCCTTGAAACCAGGAAGCAGAGATTGTGGTGACCCGACATTGTGCCATGGCACTCCAGCCTGGGTG | |
| ACAGAGTGAGACTCTGTCTCAATAAAAAAAAATTTTGACAAAAAAAAAATTCTCTTTTGTGTTATAGTTTATTTTTAATTTCAACCTGA | |
| AGGTTTATCTCCTTACAAATTTATTTAAAATAGAGGTCTTGAGCTTCTTTCTGAGAAAAAGAAATGAAGATTAATGGACATACAAGCAT | |
| TCTCCTAAAATATTTTTAGCACCAAACATCTTAATTTACATTCAAATAAATGAGAACCACCATATGCCTTTATTTATTGCAAAGCTATG | |
| GATTATTGTACTATTCACATAAAAATCACCTGGGAAGCTTAAGGACTTTTTCATATTGAACAGTTTGTACTTATGTTGTCAGAGATTCT | |
| GATTTTCCTTTGAATTTTCAAGTCAGATACCATTAGGGTATGGATTAAAGCCTCTAGTTTTCTTTGCGTTTTTACAATAACTATTTGCA | |
| TGTACCTATCTTCAATTAAATTATTTTCAGTATTTTATAAATGCCTGAGATGTTTTAGAAACACACTTTATATGATAGCACAATATTCA | |
| AAATTGCTTATAAACATCAAAAGTTATGGATTAATATGAAAATTTTACCATGTCTGATAGACCAGATTACATTGGGAGTGTCGGGGAGG | |
| AGAGATAGCCTGAGATCATTATCTGTCTATCAGA | CGTATACATACAGGTTGCTAGATCAGTATCTTTTAATTTCACATTAAGCAACTC |
| AGAGATAGCCTGAGATCATTATCTGTCTATCAGACGTATACATACACGTTGCTAGATCAGTATCTTTTAATTTCACATTAAGCACACTC | |
| TCTTTGAGGAAGGAAAGTCAGTGTCTAATGATGTGAAGGCCAACCATGTCATACTACAACCAGAAAGTGCTTGCTCCTTTTAATTGCTA | |
| TTTGTCATTTTAAAATGCTTTGTTAGGAGACAAGATTATGAAGGCTGTTGACATCAATTCTTGCCGGCCATATGCCTTTTATTGGGTTA | |
| GGAAATGTGCCATCACCTTTCAACAACATATATAAACTTATTGTGGTTCGCTAAAAAATCAGTCTATTGTTAATAAGGGTTAATTTAAT | |
| ATTTCAGACAAGGAAGAGAAATTATATCCTTGTGGAATTTTAACCAAAAATTGATTTGAAATTTCTACATAAAAACATAATTTCAGTTT | |
| TTTACTTGCTTTACGTTTTATACCAAATTTGAGAAGCCTCACAGTTTCTTTTGGTTTCTGTTTGTTGTTTTTGTTTTTAGCAGATGGCC | |
| GGAAATGTGCCATCACCTTTCAACAAACATTTACTTATTGTGGTTCGCTAAACTCGTAAACTATTGTTAAAATAAGGGTTAAATTTAAT | |
| ATTTCAGACAAGGAAGAGAAATTATATCCTTGTGGAATTTTAACCAAAAATTGATTTGAAATTTCTACATAAAAACATAATTTCAGTTT | |
| TCATTCACTTTACATTTCTCTTACTCATTGTTCACACTGTCCTGAGCCTCAGTATGTAGTTCTGTAACTGCAGTTCCAATTACAGTATT | |
| AGAATTACAGTTAATTACAGTATTCTGCTGTCTCTACCAGCTTAACTTACCACCCACTCCCAGGAAGCCAGAGCTCAATTTCCATTCAT | |
| TTTCATTTATAACGAAATACGTATGCATACAGTTCACATGCACTGACCGATATCCATCTATTATCCTTGCACATGTAGTACCTCTTAGC | |
| CATACCTTCAAATATTAGAGCAACCTAAACAGGTAAGATTAAAGGGGTGGGACTTTAAATGTTAGATTCCAGTGTCTAATATTGAGATC | |
| ATGTTAGAATGAACTATTCAATTCTCTTCCCTCTCGTGTCAGCCTCTTTGGAGTCAGCAGAGGAACATTTTTTCAGGTGCTTCCCCTTA | |
| CCACTAGAGTCTGTAGACAATAGCTCAGAACTGAATGGCCTCCTCACTCTATCCAACTCTCTGGATATTTATAAAACAATTGGATGCCA | |
| CCCCACAGAATGAGCACAGTCAGTAAATCTTTGGATGGATAAACTAATCACATTTGTGTGTGACTGAGGATATGGATACCTGTCCTCTT | |
| GCAGCTGTATTACCCAATACATTTTCTAAGTATTCTTTTCATTAGCTCAGGTGAGTGAAACACTTTTGCATTTTGTAACATTTAACACA | |
| TGCTAACTACCTCACAAGCTTAAATAAAGCATCCACTGCGTTGTGGTTTCCAGAATCGTGACATTATATATGTGTTATATTTCGTCAGC | |
| ATTTGATATAAATGTCTTAATGATTGCTGAACAGGCTATCCTTATAAGGTTGAAATTAGATGAAAGTTTTTGCTGTGGGGGCAAGAATAG | |
| CCACATGAAACTTTGGGGCATCGTTCTACTTCTTGCAGGTGTCACATCCTGACTCCTCAGGTTGCCAGTGAACCTCCATTGCTCTACCT | |
| CCATGTTCAACTCTTTTTAAGCAGTTATTTCTCAGAGAAGGTCATCAATTCCCAGTGAAATTTTATCCCTTAAAGTTCTGTGGCTCTGG | |
| CATCCTGGAAAATTTCCCAGCTTTTAAGATCTAGGTAGTCTATGAAACAGAAATCAACTGAAATTTGTCTGCATATGCCAAAGTATTTT | |
| TTCCAATATCATTTTCATAAGCATAGCACTACAATAAGAATTTTTAAATGTAATTCCTTATTATGGATCCAACAGTTATAAGGAAAAAT | |
| TGGCATTTATGATTTACCTGAAGGGTTAATGTAGTTCCAAAATTCAAAATTTAATTCATATAAAAGCTTATGTGAGTAAAACAATGTGT | |
| TTACCAAAGTGATGCTATTTTCATATCTCAAATTCAGTGAGTAAGAAGGTTGTATTC1AAGTCAGACTTTCTGACTGAATGGATGTAGC | |
| CTTGCCTTTGAGGTTGATGACTCATTTTAAGCAAATGGAGTTACTGAGATGAGTGAATGTGGATTAAAGGCAAAATTTTGAATCTCAGA | |
| ATTTAGAATCAGAAATGCATCCAGACAATGCATTTGACCACATCTTCCTGAACAGTTCTATTTTCATCCTCAGATTAAAATTTAACTTC | |
| TGAGGATTTAGATACTCGTATGTAACCATGAAATATCTATTAAGTATTGTCTATTTGACACCACTCCCAATAAAGAGATATAACACATA | |
| CGTGTCTATATTTTTCCAACTGTCCCAAACCAATTTTGTGATTAAAGATAATGAATTGTGTGTGTGTGGTTTTCGCGTTTTTTTTGTTT | |
| TTTTTTTTTAAGTTAGAAAAAGATTCTTTTTTCTTTGGGACCTCTTAGCCACAGATTTTCCCCAGCTCTGACGGAGATGAGTCATAGCA | |
| AGACTACTAATTTTTAGAACATCCAGTTCTGTTGCATATTAATCAGTGTTAATTAACTAATACTGTTTTTTTTCATGGTTTTTAGAAAT | |
| GGTCTCACTGTGATGAGTTGATCATAAACTTTAGACATTTGTGTGCATTAAGGAAATTTCCCAGACCGGGGAATTTACTTTATGCCTTA | |
| TTCATTTAATTATTTAAAAAAACCAACAGTGTTTGCTTACAAGGCATACGACTCAACTTCTAAAATTATTTTTAATATCAAATTGTGTG | |
| AGAACCAAAACAAAGGAATACCTCTCACTTCATCTCCAGGCTTCCCTTGTCTTTAGATCTGGTTAAAAATCTCCCAAGGAATTGTTTTA | |
| GGTCTCACTCTGATGAGTTGATCATAAAACTTTAGACATTTGTGTGCATTAAGGAATTTCCCAGACCGGGGAATTTACTTTATCCCTTA | |
| CTTATCTGGTAAATGTGTTGAGGACTGATCTTTGGAAATCAGGAAGTTTTTGGATAATATAGGAACAGTTCCAGATGCCTTCCCAGAGA | |
| TTTAAATCAGTTCAAGGCACCTGGACTGCTCAGAGACTGCAAGACCCTACTTGGCATAATGAAATGTAGTATAGTCTAAGTAGTGCATA | |
| CGTTTTACATTTGTTCATGAGCTCAAAGCTCTCAAAATCTCCATGTTTGCTGGCGTTCCACAATTTAATCAGTAAGCCAGTGAACCCCC | |
| ACCAGCAGCTACTCGCTGTGATGAGCTCATGGGCACATGATCTAGTGCTGTATATGCAGAGTTGCTCCCACATAGAAACTAAAAGGTGA | |
| GAATGTTCCTTAAAAAATCTTAACGTATTTTCTTTTCACCAAAAAGTGAATCTCTAAAACAAAATCATCTGCTTTACCGAACTATGTGG | |
| TTATAAACTACTAACACTACCACAAATTGATTGACATCATTATAAAGTTGAATCAGGAAATACAGCGAAACCTCCCTTAATATGATGAT | |
| GACCTCATAATTATCCTTGTTTTATTCTTTGTATACATATCAATAACTTACCTTTATAAATCATAAAAAGTTAGAAATCTGGACTAGAC | |
| CTTAAAGATGGTCTGGTATAATCTTCCCTTTTGTACAAATCAAAAAATATCACACTTCTTAGGTGTTTATCAGCCCACAGTAGTTTATA | |
| TAGTGAAGGCTTCCAGCTAACTTCATTAATTAACTATATCTATAAAATTTCTGGGTTAGATAGTATTTAAGGGAACTACCTAGTTTTTG | |
| AATCATTCTGGTAGGTTAATATTCAATCTTGAGCTTGACCATATTAATAATCATAAAACAAAAATCTTATCCTCTGAAAATGCTGAGAC | |
| AAGCTTAACAGATGCACGGTCTAGCACAGCGTCTGTTCTACAACGCTGAAACAGTATATCTAAATAAACATCTCTGTAGTCCCTGCTGA | |
| ACCAGTTGAGGAAGGAAAGTCAGTGTCTAATGATGTGAAGGCCAACCATGTCATACTACAACCAGAAAGTGCTTGCTCCTTTTAATTTA | |
| GGCAGCAGCAATTTAAGACAAGGAAGCTTAAGACAAGGAAGCTCTGACTAGAACAAGCCGTAACATGTTAAGTCTAAAGCCTAAACTCT | |
| CCACACATCCATAGCATGAGAGGTTCCATGGGCTTAGGTACCTGGCTTTTTAGCCATATCTTAGTGTACAATATCGATTAAATACCATT | |
| TTTCGTTTTGGGACCCAGTATATAGTGTTATATAGTGTGTTATATCCCTTGGGGCCCAAAAAAAAAAAAAAAGTGTGTGTGGGGGGTTT | |
| GATTTAAAAATTTGTATGTACCTTTGTTGCCCTAATTTTTAATAAGTATTTCTCAAATTAATTGCCTTAAACGTTCACCTTTGCAGAGA | |
| GGATTTCGTTTCCGTTATGTATGTGAAGGCCCATCCCATGGTGGACTCCTGGTGCCTCTATGTTTTTCTTCTCTCATTTAGTAGACATA | |
| GGTCAAAGTAAGTTTGTGGTAGCTCTCCTTCTATTTGAATTCTGGAAATTTTGATTTCCTACGATTTCCAAGGAATTGCTTTAAATGAG | |
| TACGGGTTCCCTTCGCTCCTAAGCTGAAGTGTTCACATGATTATTAATTTTTATAGAAGAATTTCTCTCCTACCAAATACAAACTGACA | |
| GATACTAAAACTTTGTTAACATTTCAATTTACTACAATGTCTTCAGCATTACCTAAACAAACTTACTATTTCTTGATCATCTTAAATAT | |
| TTTTAAAAATTGGATACTTCCTGAACTTTAGTAAGTCTTTGAAAAGAAAATGGTTGATTTTGACTCTTTGGAGATTTAGTGAAAACCAA | |
| ACAAGTGACTGAGTGTATGGATTATATACTATATTATTTAGATTATGAATTTCGATCCAGACTTTGTTATTAGCTGTGTAAGCTTTCAA | |
| TAGGTTTAACCAATACTTCTAAACTTCACTCTCTTAATGTATAAAATACAAAAATATAAATAGTATATACTTCATAAGGTCCTTTAGAG | |
| ATTAAATGACAAAATGTGTACATGTTCCATAGAGAGCCTGAAAGTACCCTAGGGCCTTGGCCTGAGCAGATGTGTGGCCCTCTTGGAAT | |
| GAACTGCTGTGTATATAGTGCCGCCCCGGTATATAAAAAGGTTTTTCCCCGGTTTAAGATTATATCAGAGAGAGATCCCCGTATATATA | |
| GTTAATATGAGACTTTTAATTTCATAATATGCTATGTTTCATAGACTGACTGATGATAAAACATAGACTCATAATCATTTACCACATAA | |
| CGCAGTCAACAATTGTAATTTAGTTTTTTATAAAAGTTATTATTTAGTCAGTAATTCAAGGCTTGGACAAATTAGTTCTCACTATGTCT | |
| GTGTGCCCCTCAGGGTGTAACATACTTTTCGACTCAGTCTTTTTATTGGTTTGTTTTTGTTAAAGTACATGACTCACTACAGCAGGCCC | |
| TCAGCAAAGTTGTGATGTAAAGAAATCTGGCCGGGCTCGTTGACTCATCTCTGTAATCCCAGCACTTTGGGAGGCCGAGGCTAGCAGAT | |
| CACAAGGTCAGGAGTTCGAGACCAGCCTGGCCAATATGGTGAAACCCCATCTCTACTTAAATACAAAAATTAAGCTGGGTGTGGTGGTG | |
| TACACCTGTAGTCCCAGCTAGTCAGGAGGCTGAGGCACAGAATCGCTTGAACCTGGGAAGCGGAGGTTGCAGTAGACCCGAGACTGTGC | |
| CACTGGACTCCATCCTCGACAACAGAGCGAGACTCTGTCTCAGGGGGAGAGAAAGAGACCCTATATACCCGGGCCCCCCCCCCCCCTAA | |
| AGCCTTGCATTTTGTTTCTTCCTTTTCCTCTTATCCATCTTCTAGGCCCAGGCCTGTTAAATTTCTTATCCTTATTCCCAAACAAAGAG | |
| CAAAATGTTCTGACGTCCTTCTTTACCTTTCAAGTGAAACTCACCCATTATTTAGTATGGGTACTACTAGTCATAGTTGCATTTTAAGT | |
| CTCTTAGCTCCAATTGTGGAATGAAAGATGTTTTTTCTATTAAAAGGTAATTAGGCGGGTATATGATGGCTTAAGTCCACAGAAAGCAG | |
| ACACGGTCTTATGGCCTCACATCCTTTAGAACCTTGATGAGGAATGAGGAGCAGCAATTCCCCTTATATATACCCGCAGTGAGAGTCAA | |
| AAGACTTGCCAACTGGTTTGTATAACATTATCTCTTTTAATCTCTTACAAATCTCAGTAGGTAAAGTGCAATCCCTGTGAACCCCAAAA | |
| CAGACTCAGAGAATGGAATTATAATAACTGGCCTTTCTCACAAGACTAGTGGCTCTATATCCTGCAGCAGGCTATTGCTCACCGAACCT | |
| TCAGAGTATATATATGTTTTGGGGTTTGGGGGGCAGGGTTGGGGCCTTTAAATCAGGTATTTGGGAAAAGGGAGAGATCTTATATACTA | |
| ATGATAACTCTTGAAAACAACGGAGAGAGAGGCTCACTGGTCAATGGGTACTCAGGTATTGGGGTAAGCCGGCCCACCACTTACCTTTT | |
| TAACACATGTGTCTGTGTGTCACATGTATAAATATTGTGCCTTTATGTATGTGTATATTTGTGAGAAGGGGATTGAAAGACACATGTTA | |
| TCAGCATTGACACTGACTATAATGACTGAACAATTAACTTTAAAGCGAAAAAAACTGGGAGTTTTTTCTATACCTTCCCACAAAAATAT | |
| AAATTATGGTATTTTGTTTACTTCTATTGCTCTAGTATTTTGTGACTAAGTAACAATTCTTGATACTAACAAACACATGGCATTAACCT | |
| TCTTATAAATTTATTGGCTAAGAAAAAACTTTATACATCCATTTATTCCCAGTCTTCATACATCTTTCTCATGTAGAGGTTTCATCTAC | |
| TCAAAAACATATTGCTGCCTCAGTCACAAAATAATTTACAAATGAAACAACTTAGTGGGCATTAGATGTTCTTGTAGAATGAAAGTTTC | |
| TATATCAAGGACTTTAGTACTTACTGGGAAATATACATATGTAAACGACTAATAATAAAGCAGGTTCAACTACAGTCACATTATTTCAC | |
| CAAATCATTTATTTTTTATTCACTAATTCAGTATCCATTTATTAAGCATTTATCAACTTGTAAAGACCACCACCAACTTAGAGGTTACA | |
| GAATATGACTATACAACCTGTCATTGTTCCCTTAGCTTGTCATTGTTCACTCAAAATTGAAATGCTTCTGCTGTTCCTAACACTTATAC | |
| GAAGTAATTTTATTTATCCAAGAAATTCTTAATTGCAAGGATGGCTAGGAGAGAAAGAAAGAGACAACGCCCACGTATTTTTTTTTTCC | |
| GAGCTCTTCTCCAAATATCCCTTTTAATAGTTATTTCTGCTCAGGATGACCTTTAAGTTTACAAAGGAATTTTAAATCCCCTCTTCCCT | |
| CTAATGTTTAAGCAGAGGAAAAAATTGTTCTCCATGCATCTTTCTAATCAGGAAGACCTTTAAAATTGTGCATGTCTTTTCTGTGGTAC | |
| TTTACTAAGCACCTGCTCGGCGAGAGACTGTAAGGGAAACAGTGGGATAGATACATCTAAAGTTTCTCAAACTTGGCATTATTGACATT | |
| TGGGGCTTGAAAATTCTGTGTTGTGGGACGCTGTCTGACACGTTATAAAATATTTAGCAGCCTCCCTGACCTTGACTCACTAGATGCTA | |
| ATAGGATTCCTATAATTGTGACATCCAAAAAGTCTCCACATTGACAAATGTCCCCTGGGGGGCAAAATCACCCCTCTTTGAGAACCACT | |
| GATGTGGATGCAACAAAGAATCAACCTTAACCTTACCCATTTGCATACTTTTCTACTATAAGTTGTCTCACATAAATTACCTACAAATT | |
| TCCAAGTGTATTATTTTATAAACGTGCCTTTCTTGGTTATTAGTAGATATTTCTTATTTCAAGATGGTGATCATGGTGTCTGTATAATT | |
| TTCTGTTAGAATATCAGAGTTAGTGTACCTGGATACTAAGGAGAAAGATAGAAAAGATAAAATATGTATCTTCAACATTTTGTATTCTG | |
| TGATGACCCTAAAAGCTAATGTGGGAGTTCTATTGACTGTTATGAAATGCCATGGGCTGTGCTTTTCCTCAGCTACAGCCTCTCTCCCT | |
| TGTGTGAACAATGGGAGGGTCTATGGAATGATGCTTTCAGGTGGGCAGAGGCCTCGTGGAGGGTGGCCCCACACTCACCCCCAGAATCC | |
| AGAAGCATTCCCACAGGAACTTCAGAGGGAGAGCATTTACATTTACAGGAAAGAAATGATATCTTCTGAGCCTACATTTGCCACATATT | |
| CATTAAGTGCATTTCTTCAGGCATTACACTAAAATGCCATGGGCTGTGCTTTTCCTCAGCTACAGCCTCTCTCCCTCCCACCTCACACC | |
| CTCTCTTCCTGTCATGCCTTTGTGAGAGCCCCACCACTCTGCCCTGCCTGGACAGGAACCTGGAAACCCTGATACTTCACTGCATACTC | |
| CGTCATATGGAGACAGCAGGAAAGAGTCAGGGGCCAACTTGAAAAGATTTGGGGCCTGGCTGTGGTTTGCAGCCTCTAGACACCAACTA | |
| TACTTTATAGAACATGTCAAAATATAGCCTGTATGGGTTACTCTTTACTGATTGTCTAGATTTGATGCCTTACATTAATCATTTAAAAA | |
| AACCAACTCTAGAAGTCTACTCAAATCCTGCATTTTCTAGTGGATAGTGTCCCATGCATAAAACCCTGTTTCGCATAGAGTGTAGGCTA | |
| GCTTACAAGACCCTCTTACCATGTTGAGATTATTTCTAGAACTCGGCTACTGAACCATCTGGTGCTCTGCCTGTCTCTTTCACATACCT | |
| AAGAAGAAAATTTCCATTTAGCTTTCTGATATCTTGCTGTGCAAGTCTTCATGCTGCCCCCCCAAAAAAATTTTAATTATAAAATTCTT | |
| TCAAGCATATAGAAAGTTTAATTGACATAACAGACACTATGTACTACAACACAAATTATTTAATAGCTATTAGCATCTTACCTTATTTG | |
| CTTTGATAGCTCTCTCTCTCTCTCTGTCCTCTCTCTCTCAAATAAAATAGTTACAGCTAAAACCCCACTTATCCATCATTTCTGGTTTG | |
| CCTCCTTCCTTCTCTTACGTAAGCTGCTACTCTGAACTTTCACTCCCCAAGATCTTTTTATATTTTTACTACATAAAGATGCATCTATA | |
| AATACTCGCATTTTCAAGAAAGTTAATTTTTACTAACTTCAACCCAAATTTGCCATTGTATAGGATGGCTCCTTTTCGGGCAAAACATA | |
| GGTTCAAACTGGACCCCATTGAAGATTAACTGGTACTCACTTTGATCCTATTGATTCCTCTTTCCTGTCTAGATAGAGATCTATTTCTC | |
| ACAATGTAGTTTGTACTAACTCTTATAGCAAAAAATAATATTCAGAGAAAAATCTAGGGGAAGTAAGTATGCAGACTAACTCTTATATC | |
| TTGTCCTTTCATAACAAA7kATTTCCCATCATTAAACATTTAATAATCTATTATACTGCCTATAAAATTTCCCTTTAACTTTTTTCCAT | |
| TCTAATTTCATCACGGAGCTCCTAATAGGAGAGGCTGGCAAAAAACACAGAAAACCTTCCTCTAGGCCAACCTTGTCCCTGCCTGCTTT | |
| TGACTCAGCAAGGACTGCCAGCCAACGCTCACTTCCCTATTTTTCCCGAAAGCCACGCGTCAACTTTTAAAAATCAAACTGTGTTCCCT | |
| TTCACACAGGTCGCTGACAAGAGAACCACCCCCATCCCACTTCAGAAACTCTCACCAATCCTCCTCTAGAGTGACCAGACAATACCCCT | |
| TCCTATCCTAACTAATGTGACACGTCCTTCTGTGTGGTCCACTCTATGCAAAACCAGAAATTGACAACAATGACTTTCATTGTAAAGTA | |
| CAAATAGCTTACCAGTTAGTATTCTTAGTTTCTCTTTACTTATAAATATCAAATCCAACGCACTTGACCCACCTCATAGAAAAGTCTAA | |
| CTATTTTTAATCAAACATTTTCTGAAAGCTCCTTCTCACATATGTTGTCTTTCCTGAGTGTTTTATGTTGAGTGGAAGACGTGTTGAAA | |
| TGTTTAGGTCAACAACAGCTTTTTTGTTGTCCCCTTCCACTACTGATTTAAAAAAAAAAAAAAAAGGCTCCACTCTTCCTGCCTTTCCT | |
| TAAAATCCTAAATGTTAAGTTTCTGTCGAGGTCGAATAGAGACTACCCGATCTGCTGTCGCATTTGAGCCATCACATATCCTTCAACAT | |
| AATCTCCCTTTCCTTTGTATAGACTTGTCTGATTCACGTGGTTTGGATCTATTTGGAATACTAATTTAAAATAATTTAACCTGACTCAA | |
| TTACTTAAAACAACACGCGAGAAAATCACCAATAATCAGCTATGTGTGCGTATAGAACCTAGTTCATATCATTTATTAGGATTTCCATT | |
| GAACCCCTAAGCTGTTCACTGCGCAACTATTTAATTACGCAAAATAACCATACCCCTATGTACATTATACTGTGTGTTTTGTAAACCAT | |
| GAGAAAAGCTGCTTATTGAACAGAAGGGCTTTGTAACCTGGGGTCTATGGATCCCTGTTGGTGGTTTCAGACCCTTTTTAGATTCCTAA | |
| ATTTATATGCACACTTACACATGTATGTATGTGCATTTTTTCTTGGGAGATGAGTATAACTTTTTATCATATTTTCAAAGGCAAAGATT | |
| TAACATGGTTAAGAATCATGCTATAGCTTGTATCACTTTAGCTTTTTCATACCAGCTATCTTTTTAAATGATCTATGTATAGTTTTTTA | |
| AGATTTTTTCTTTTTTTATTCTTGCATTTTTAATCAATTTTAAATGAAGTGAAATGCTTTATGTTGCTCAACACATAGCCCAAAAAGCC | |
| TCCCTTCAGTTTTCCAGTACCTCGAAAAAACATCAAACTCAATTGATCGCCAAAAGAGTTGAATGGGCTTCACTGCTTTTTCTTTCTTG | |
| CTCTTCCATACATTTGCAGATTAAGCATGAAGCTCACCATTAGTGATTGAAGCCAAAAGAGAGAAGGCAATTCATTGAACAAATATTGA | |
| TTCAGCACTGTGTCTCACGTACTGCTCTAGACAAGCAGACAAAAdTCCCTCCCATATGTAACCACAATTGAGACTTATAGGTAATTTTT | |
| GTTGCAAGTAGTTCAGAAGTTTTGTATTTTGTCCTAAAACAAAATATTACTTTGTAAAATATTTTTTTAAATAGCCCTCTAAATCCGCT | |
| GTATATATTCAAAGTCACTATTATAAAGTGGAGAAAGCTACTCATTAGACAAAACCTATCACACAGATAATAGCTGGGAAGCTTAACCC | |
| TTAGAAATTATTAGGAGAGATATACACTCCTTGAATTTAAGAACTGTCTGAATTTTAAATTTTGTTTTCTCACTTCTGGCTTGGTGACC | |
| TTCCTCTATTGGCCATTCCCATTTTTCTGCCCTCCACTCTTCCTTGACACATGATTATTTCTGGGACCACTGTTCAATCCGAAACAAAT | |
| CTCTGGTGGCTTCTTTTAAACCCAGTGCTCTCAGTCTGAAGGTATAGTGGGGCAACTCTCGGCATTCCATTCTCGAGTCTTGACCTCAT | |
| AAAGTCACATATTGGCAATTCGCCTTGATAAGGTCAAGTGAACACACAGAAGCACTAAGATCCCCACATGTGTTCCACTGCCTCCTCCA | |
| CACACAACTTGTTTTTCAACCCATCTGCAGGTCCAGAGGTGAAGCTGGAGCCTACCCTGCAGTCTCAGTCTTTTCTCCCCACCCTCCAG | |
| ATGGATTCCAAATAAGAAGCCTCCAGACTACCTTCACCTCCCTGGGGTCTCATGGACATGGGCTGCAAAGTCCTTGCAGACTTTCACAG | |
| ACAATGCTTTTTGCCTTTAGGTCCCCTTCTACCCAACAGAATGATTATAATAATGGCTTGATGTGCTTACTTACATCATCTCATTTAAT | |
| CATCACAGTAATCCACTGATGCAGGTTCTGTGATCCGCATTTTATAAATGAGGAAATTGAAGCCTAGAGATTAAGAAAAGCTTAACTTG | |
| GGTCTCACAGGCTTTTAGAGATGGAGCCCAAGATTTCAGTTCATGCCATATGGTTTAAGGGCCTGCACTCTTAACCATCGTCGAATACC | |
| ATTCATTATCATAATGTTAACGATCTGTTTCTACTTTGCAGGAATCTTTTAAATCTATGGTTCACAATCATGGGTGGGCTTCAGAATCG | |
| TCCAAAAATGTTTCAACCACAGAGACGCCTGGGAACCACTGCACAATTCCTAAATCACAGTGCATCATCTAGCCAATCTTGGATTCTAC | |
| AATATTCTATGACAGTTTCTAAGATACATGCCATTATATAATGGCAACATATTACAACAGATAATTGAATAGATATATTCAATGTATAC | |
| CGTGGAATAGAATATTATGCCACTCTGTTTTAAAAAGATAATTAAGAGGATACGGCACAACGTGGAAAACTGCTTATAACATAATATTA | |
| AGGATAAACCTCAGCTGGGCATAGTGGCTCACGCCTATAATCCCAACACTTTTGGGAGGCCAAGGCAGGAGGATTGCTTGACCCCACGA | |
| TTTTGAGGCCAGCATGGGCAACATAGTGAGACCCTGTCTCTATAAAAAAAATTTTTTAAAGAAAAAAACACAGATTTCAAATTATTGCT | |
| AATATTTGTAGTGATTACAAATATTAGCTTCTTTTTATAAACCCTCCGTGGAAGCATATAGATGTATCTTTAGCTTTATTTATTCATCC | |
| ATGACTTAAAGAAGATTGGTTAACACCTGACTCCACCTTGACTAGCAGAACATTGGACTGAACGAAAAAAAAAAATGTGAAGCTGTCTT | |
| ACTTAGCTACAGTTTCCAGCATGTTTTCTCAGGAGCTCTATGGGAGACAGTGCAGTGTGGCTCTCAAACCTTACTACCCATAAAAATCT | |
| GCTTCAAAACCTACTTAAAATATCGATTTTATTCCATTTCCAAACCCTCCTAGAGTAGGTAATTCTCCCACTACAATACACTTAGTAAC | |
| ACTACAAAAAAAATATAACATTTTCTTTAATGCATAGCTGAGTTTATTAGAAAGTATCAAAACTACTGGGGCGCCGTAAGACAAAAGAA | |
| AACTTTGCCAAAACACAATAGGATATGGGTGGACTTTGGGGCCACAGCTGCCCTGGAAATATTTGCTGACCTAGATAATCAAGAAGCTT | |
| GGTTTTTAACAGCTTCCTAGAGACAAAAGATAAGGCCTTGGGCCTTTCCAATCTGAGGAATTCAAACTCCAATCACCCACTTAATTCAG | |
| GACTCTCTCAGGGCTATAGTTTCTGTTCATGGGTGATCCTCAGGCAGAGAGGTGATGAGGAAGTTGCCTGTCTCAGCCTAGGGGGAATC | |
| AGAAGATAAAATGCCTCTCTTGAGAATCAATAACTACAAGCCCAATTCTTACAGATTCAGAGTTAAGTGTATGCTCCCTGAATAGAGTA | |
| GGAAACCCCAAGTCTAGAAATGAATATGAAGTTGTTCCACATTGGGGATACCCTCAGAGCCTTGCAGAAAAGCATAAAAATGTATCTGG | |
| CTCAAAGAATCATCACCTCCTGATCAAAAATTAGAAAATATGTATGCAAATAAGCCACTCTCTTTAAGAACTGGCAGAACAATGAAAAG | |
| CAGAATTAAATCCTCAGAATCTTCAGATAATTGAATTACCAATTGTTTGATATATAAATACAAAATAAGTTTCGAATAGAAAACAGACC | |
| AGGCACAATGAAAATAAAACCAAGTCGAATTTTTAAAATTAAATGTGCGACACTGAAAATGATTCAACATAGTTAACATGGATAATTTG | |
| AAACAAAAATTATGAGCTGGCAGGTACAAAAGAAGGAGTAACAGGTCTCTACTTGAAGTCCTAGAGAAGTATTACAACTGCAGATTTCT | |
| CGCTCTACATTTACAGTTTCTAGGTCTGGGGTGAGGTTTAGGTGGTTGTGGACCACACTTTTAGGTTTTAAAGCATTGGCTCTGAAGTC | |
| ATTTGGTTCAGGCCTGGCCATGATGTTGCTTCCTAGTTTTGCGACTTGCAGTGTTACTTAACTTGGCTAAGCATCAGCTTCCTTATCTA | |
| TGAAACTGAAAAAATAATACCTATGTCATAGGATATATGTGTAAATCAAGTTATTCAGTACATCCCACACAGTAAGCACTTAAAAATGA | |
| CAGTTGCTTCTATTTTTATATAACCAAATACACTACTACAAATATTGAGTGGCAGCTTTGCTGGTTCATTTATGGATTCATGGAGCTAC | |
| ACAAAACCTTCACCAGTCTATTAATTTATTTTTAGTACAGTCTCTCTAATCAGTATTGCTTCCACTGTATATTGACCCCATATCCTTTC | |
| TTCTGTCTGGGGTTAAGATAGGAGACTGAGATCAGCTGTGTCAAAAGCTTTCTTTTAGATGTAACACATGATTTCTTATGAAAATCTCA | |
| TACGTAGGATACCAGAAAGGAATTTATTAAATTCTTGTTAGGCTCCTTAGTTGTATTAGTTTTGTAGGGCAGTGTTTTAATTTAGGATA | |
| CTTATCATAGAGAGCACCCCTTCCATAATCAGCAGAAACACAAGATGGAAATAAAGATTTTATTACTTAGTGGTCCTGGCGACCACACA | |
| GCACTCCAGGGAAGGCCACACACCAAGATCAGGGAGCATGTGGAGAAAGAGAGAGAAAGGAACTCACGGGCCATTGCCTTTACTGGCTC | |
| CACGACATTATCCAAACAGGTTTCCCACAGGGAGTTTTAATACGTGGGTTTCAAGCAAGCAGGTATGAGTTCCAGGAGGTCACAGTGTG | |
| ATGAAGAAGAAGTCACTGTGGCATATCTGCACAGTCCATGCGAGGTGTGAGCATCAGTGAAGACCAGTCAGGTAGGCTATATGTAGCTG | |
| GCCCATCGGGAGGCGATCACCAGGTGGCAATTGTATAATGCAGATATCTACATTGGCCACATAGAGGAACCGGGAGGAGGTCTACTACT | |
| GGAAACTGCGTCGACGGTCACTGAGCCCTGCTTTAGGTATGAGAAAGTCCAGCTTATATTCAGAATGGATGCAAAGGCAGCATAAAATT | |
| AAAAGCATTCACTGCCAGCTGCCATAACAGTTACCACAAACTAGGTCGTTTAAAGCAACCGAAATATTATTCTTTCACAGTTCTGGATG | |
| CTAGAAGTCCAATATCATGGTCTTCGCAGGGCCGTGCCCCCTTTGAAGGCTCTAGGCAATAATCCTTCCTTCCCTGTTTCTAGCTTCTT | |
| GTGGTTGCTGACAATTCATGGCACTTTGTGACTTGCAGCTGCATCATTCCAATTTCTGTCTCCATCTTCACATGAATACTGTCTTCTCT | |
| CTGTGTCCCTGTGTCCAATCTCCCTCTTCTTTCTCTTAGAGACCTGTCATTGTCATTTACGGCATCCATAACCCTATGTCACCTCATCT | |
| TAACTAATCATAATTGCAACGACCCCATTTCCAGATAATACAATATTCTGAGGCTCTGGGTAGATGTGAATTCTGGGGGTACACCATTC | |
| AGCCCACTACAACATATTGTCGCATTGAGAATTATAAGCCAGTAGCCCTACAAGTATAGGATGATGCCCATTTCAGCCTGATAATACAC | |
| TCCGAAAGAGATCACATGGTAACATTGTTGCCTCAGTTAATCATAGAAATACTCAATTACTATAGAAGTAACAAGAAAAATGCTTTGTT | |
| ATTCTCATGTCTCATTTATCGAGTTCTTACTATCTATCTGTTATTGTTATCCTTATACACTTCTGCACAAAAGATGGTTTTATCCCAAC | |
| TTTTAGCTTAGAAACTGACCCTTAATCACTAACTTGACCAAGAATGGCTTCTCTTTCTCCATTTCCTTCCTCTCATGGTGTTGTTTTAT | |
| ATACATAAAATAATATATATTATTCTATTAATATAGATTAATATATCTATTTATATATCTACATTTTATATATCTATAAAACTTATGTT | |
| GTAACTTTTATATACATATAATATATATTCTGTTTATATATTGAATTTATATATATATAAAACTTAACACTATTAAGTCTTAACACTAT | |
| TAAGTTTCATGGTGTTCAGTTATATAGTTCTATTAATTATATATATATCGGCGCACGCGTGTTTCTCCACCCTACACTTCCACTTTGAA | |
| ATCTAGTCTCCAGCCTCAGCCACTTCTTTCACATTGCCACTTCCATGTCTGGTAGACTTAACATATTCACAACTGAATTTCAAATCTTC | |
| CCCACCAATCTGCTCTGCCTCATAGCTTCCCCTTTCCCGAATGTGGTTGCTCCATCCTTCCAGTTCTACAGGCCCCAAATCTTGCAGTT | |
| TTCCTTGACTCCTCTCTTCTTCTCAAATCCCACATGTAATCCATCAGGTAGTCTTATTGGCTGTGCCTTCACACTTTTTTCAGAAACTA | |
| ATTATTCTTCAGCACCTCCAATGCTGCCACCCTCGTATAAGCTACCATCCTCAGCATCTTTCTTCCACTGGATTTTAGCATCCATGAAT | |
| GATTATTTCCTAAATCAATTATCAATAATTGTTATAAATTTGTCATTTTTCTATTTCTATCCTTCCTGTCACTTTTCCTGGTTTTTTTA | |
| AAAGAGCTTTCTTCATTCTTCTTTAAAAAAAATTAAAATTAACATCACTATGAACTCCTGGATTCTTTTGTTATTCATTGTACTATAAG | |
| CCATTCCTGTCATTATTCATTTTGATTCTTAAATTGTCCTATTCTTGGCCAGTGGGAGTCTTTCATGCTAGCTCCTATGTCTTTTTCAT | |
| ATGTCCCTATCTTTTTTTTCCTAACACTTTCCTTATTTCTGAAATTATAAGCTATTCTACGTTCACCCTATACCTTCACTACCCCAGTC | |
| CACCCATCCTCTCTCTCTCTAAGAAGTCCTAGTTCCTTTTAGCAGGAGAAGTTTTTCTAGACTCTTTCATTAGGCAGAGCTACAAAATT | |
| GATTATTTTAAAAATCTCGAGTTCATACACTAATTCATAATTCTAATTCCAATCCAACACTCCAGGCTTCTTCCCTCCCTCCCCTCATC | |
| CCATACTTGTATTTGGATTTGAGATATCAAATGTGTATCTCATATCCCATATTTCTTCAGTAACTGAAAACCCTCATCATCAATGATAG | |
| CAGCATGCTTACTCATTTTGCTCAGTCCAATAATTCACACACATTTGTAGAAAAATAGAATAGTTTTAGAATGCCTATACCAATATCTG | |
| CAACCTTACTATATTCGGTTCAAAATTTCTTTGTAGTTCTTTTGACCAAATGTATTCAACAGAGAGCATTCAGTCAGAGTAATGTGTAC | |
| AAAAGTTACTTGGATTAAATTTTTTCCTTCTGTGTATTTATGTTTCCATTGAGCTATAGAGTTAAGCTATTTTTTTATTTCTATTCCAT | |
| TTTAGGGGTTTTCCCATCCCTGTTGATTTAGTAATTTTATTTTTTGAATATCAACATTTCTCAGTTTCACATCTTTCATTTATATGAGA | |
| TTAACTAGACCCTTTAGAAATCATTATAAAGTCAGGTTGTTCTATTAATTTTTAAAATAGGTGTATAAAAACCAAAGGGATCAATCACA | |
| GCCTTGATTTAAAGTACGTTCTGAATTATTTCAGACACCCGAATCCATCTAAACCCAGAACATTTCATTTGTAGATTTCAAACACTTCT | |
| TGGCCTTCCTACCTTCCTTACATTTATGTAGCATGTGTTTATCCAAGGCACACCACCTGCCAGGCACAGTGCTGGGCTCTGTGTTGACA | |
| CACATGAATGTGATCATCTTTCTGTAGCCCCCAGGTTACATGGCACCACAGCCAAGATACACACATGTGTGAGAGAGTAATCAGGGTAT | |
| TACTCAGGATTTGCTGGAGGAATCCCTCTTCAAAGGATATTCTAAATTGGTGTCTCTGTGTGTGTGTCCGCCCGCGCGCGCATGTGTGT | |
| GTGTGTGTGTGTTGAGGTGGACTGGCAGAAAATATACCCTCCTTGAGATCACCTCCTGGCTAGATAGTCTCTCCCTTCTCTGTTTGTGA | |
| TAGAATGCAAGTGGAATTTGACAGGTGTACTTCTCAAACTAGAAACTAGAGAGCTGCTATCTCAGTCACCGTAAGAAGAACGAATCACA | |
| CCAAACCTACATCATTGTCTTCTATCACAGGATTTCTACAAGGGTGAGATAGTGAGATGCTGGAGATCCCTGTTTTGGTAGTTCAGGGA | |
| TTCACAGCATCTCCCATGCTGGTGATGTCAACCTGAAGAGATATTTCTAGTGATAGGTTCCAGGGATCTTTCACCGAACACTTTAATAT | |
| ATTTTATCATTGTCCTGAATACAGCCCTATAAGGTTCTTACTAAATTTGCAGGTGACATGAAGTGATAGACAACAAACAATTTGAATTC | |
| AAATTAATTTTQAAACCTGAGACAATGGACCCAATTCTTCAGCAATTATATTTAAAAGATATAAATACATGCAGAAAACAAATTACAAG | |
| TACAGAGTGGAAGAGAGATGATTTAGCACCAACGCATGCAAAGAGATCTAGGAGTTTTAGTTAATAACAGACTCAACATGAACCAGCAA | |
| TGCAATGTGACTACGAAACAAGTAATTCCTCATTAGCCTGCATTAATGGAAACACGATACATAAGACCAAGGAAGTGGTGGTCTCATTT | |
| GCACTCTGTGATGAAAGGCCACACCAGGAATACAGTGTAGTACTGCTGGGCACCACATTTGTAGAGAAATAGAAACCAACTTGAGTGTC | |
| TTCAGAGCAGAATGGTGAGAGAACTCAAACTTCATTCAAATGAGGAGTAGTTACTGAGCCTCAGAAATTTATAGCCTGGAGGATTTGGT | |
| TGTCTTCAAATATATGAATGCCTGTGATGGGGAAAAAAGGATTAAATGTGTCCTGTTGTCACAAGAGGATGAAATAAGGAGCGGTGAAT | |
| GGAAGCCCCAGGCGATAGAGGTTTCAGTTGCTGCTGACAGTCAGGGCTGTCTGAGGATGGAATGTATTGCCTCAGGACAAAGTGAGTTC | |
| ATTGTCACTGAATTGGTTCAAAAATGACCAGGAAGACTACAGGGCAGGCAACCTAACTTGTATTACAATAAGTAATTTTTTTCTTTTTT | |
| ATTTGTATGCCCTTAGAAAGATGTTTACTTAACTTGATAAATCTGTAACAAGGGATAATCAAGATTTGGTTATAGGACAATTACAATTT | |
| TCTGACAGATCCCCCAAAATAATAGAAATTAATAGCTTACACACTACTTTATCCAACCTGTAGAATGTATAACTTACTTCATATACTTT | |
| GTGTTTACTCAGTTTATACAGGCTGAACAACATAAAGTTAAGACATAAAAGGGTTTTTTTTGCAGGTTTAAAGTACTACCATTTTTCCA | |
| AATTACTGGCTGATCCTAGAACTTTGAGAAACTTTGGTGTTTCCCCCCTTCCTACTTTTGTTTACATCATAGTACGTAGATGGTACAAT | |
| ATTTTGGTTAACCTCAGTCTGACACTTAGATTGTGTTCTCTTTTGCCCTATTAATGTCTAGAGTGTAAAAAGACCAGTAAAGTAGCACC | |
| AACAAATCAAACAAAATTTCCCACTTCTACTCACTTGTAATTCTCTTAATTTAGTAGGGGCTTTCCACAGGAAATCACAATAGAATAGT | |
| TGAAATGCTCTTTATCTTATGTACTTTCCTTCTGAAGAAAGGGACAGTTAATTGGCTTTGTTCCCAACATGGTCCAAATTTTTATACAG | |
| GGCAGCTGTATTTACTGTAGTACAAAGGACATTCTACACCCAATGATGTCTCTAAGTATTTTCAATAATTAAGAACCTAATTGCTTCTC | |
| TACCCAGTGCGTTTCAGTGAAAGTGATAAAATAGATTTTCTAAGCAAGAAAAATACATAATGAGGGTGGTTGTATACCTTTAACAAAAA | |
| AGAAATCAGTGATATTCAATCCCAAGTAAATTTGGATATCACAGAGTCAGAGATATTCTTAATTATTTTAAATTAAATTTGAGTCAAAA | |
| ACAATGTGTTTAAAGACTTTAAAATCATTTTAAAAGAATGTTAGAAATTTACATTAATAATTTCTATTTTCAATGAATAAATGAGAAAC | |
| CTAGAATTTTTGTTTTGGACACACTTTGATCATATCTAATGTGTTCTACTTCCTCTAATTATTTTTATCTTATTAGGATTTTTAAAAAA | |
| CCTCTTAGATTAAAATTTCTCAGTGTATCTTTGTTTGCGTGTCTTTTTTTCTCTCTCCACTTATCTAATTGAAGCATTTCTGACATATC | |
| CTCAGAACACACAGATTCTTATCAGGACTTAACTACCCAGGGCAGGGCCCTCCTTGGATTTACTGAAAATAAACACTTTCGGTACTCAG | |
| TCTCAGTTTTAATGACAAAAGTTGAAATCTTTGCCTAACATGAAGGTTTTTCTTTGTCTCTAAATTTTGATTTTCCATGCTATCAGAGG | |
| TCCCATTGCAACAGGTAATTTGAGTCTTGATTGTATGTTTTCTCAGTTTGTAATTGTGGTGAAATGTATTTAAAATGTTCACAATTTTA | |
| AAGTTAAGTTTACCTATAAGGTTCTTTCCAACTCAGAAATTCTGTGATGCTAAACTTTTTTTTTCTAGTTTGAAAATCTCATCTTTGGA | |
| TGTTGTCTTCTGTGTCCTATGCATATGACTAGGAATCATAGAATTTTAGAGCTGCAAAATGAGTGAGACATTATCTAGTGATTCTGCAA | |
| ATGAAGAATTAAAACGCCCAAAGAGGTTAGGTGACTCACGCAGTATCATAGCAGTACCTGGTGACTAGAACTCTGGCCACCTACTCCAG | |
| CACTAAAAATTAACCCAAATGTTTTTATCTGAAAATTTGCAGACTTCAGGCTACCCCTGTAAATAAATAAATAAGAAGTTAAAACATAT | |
| TTGAGAAAGCTAAGCTTAATATGCCACATTTGGGAAAACATGTGCCTGACACAAATGTATTTGGTGTAATAGAGAACATTCTTCCATTA | |
| TACCCAAATGAAAATTTTGAACATTCTCAAAATTCTTGAATTTTQAGACATTTATGTTAAGCACAGTTTATTCAGACTCTCTATAAACT | |
| GTGCTGTCATCCCTGTTGTTTTCTCCAAGCGGAATCATACTTAAATTACAACTTTACGCAGTTTAATGTTTCCTATTTTTGCACTCCTG | |
| GGATATTTTTCAGTGACTCAGAAGCATAAACTGACACTGTGAAGTTAACGCTGTGTTCCAGTGGGAATGACAGCATGTCTCTAGGACGC | |
| TCATGCAGAAAGCCCTCCTCATGCAGCTGTCTTGCGGAGTCTCCCTGCTGGTGGTGAATGTGAGATCACACAGGGCCAGCAGCCAGGGC | |
| ACTACATCTCTGAGTCTCGCTTCTGGTTTTACCTAGAGCCAACCCTTGGTGATTGTTTCCCTCCTTGGCCAACCCCCCACACAATACGA | |
| AAACTCTCTCTCAAGGTCCAATTCTATTAATACCTGAGAGGTGAATTTATAAACATTTAAGATGTTTTAGCATCTCTGCAGCCAGTGTC | |
| TCTCTAAATGAAAATTGCTTCTCGAAGTAGAAAAGCATAAGGGAGTACAACTGGCTTCAAAAAAAATCCTTTTAGGAATAAGAATATTG | |
| CCAGGGTTAGGTGTTCCTAACTGGTTTCAAGCAGAGGAGTTAGTAAAATTCCAAAACTTAATCAGCTTCATGTACTCAACTTTTGCTAG | |
| GACTTTCCAGAAACTCCTCAGGGATCCCTCAGAGTTAGATTACGCAGATCAACCCTGCCAAAGGACAGTAACCATTCAACAGCTGGATA | |
| GCCTGTTGGGCCCCACTGTCCTCAGCCTTTCCTAATGTTGCCTGGGAACACCACTTTTCTCCTCCCTCCTTTTTCAGTGTACCCTGCAG | |
| ACAATTATTTATTTATTCACTACCTCTTTTAACAAATATTCATTAAGTAGGTACTGGTTCTCTTCTAGGGGTTGGAAAAACAGAGAAAA | |
| TTAAATAGTGCCTTTGTCCTCAGCAAACAATCCATTTAATAGAGAGACGAAGATATGCCCTTAAATACCTGAATCACCAGCATAACCAG | |
| CACCTGCGGCTGCCATGTGGGAATTGTTGAACAAGATAATTTTTGAATGAAAGAGTAAATGACCTAATTGCTCTAATGTAAACATAAAT | |
| TTCTAAGACTCGGCTAGTTGGGATGATTATAGAAGGATTCAACGGAAAGATGTATCAAACCATAGATCTAACCAGAGAAAACAGCACAC | |
| CAGAAAAAGCAGCAAAGGCTCAAAAGTACAAATCTCTCAGATCCAATCCACAATAQTAAATAATTTGCTGCAATGAAGAGCACGTGGAA | |
| GGCAAGGGTGGTAACAACCCTCCACATAGTAGATAACGCCAAATCATGCAAGGATTTAAACTGTATGTTTTTAGAAAATCGGTAAGCTA | |
| TAGAAATTTTATATACGAATGAGAAATTATCAGCAGCATTGTCCTTTAGGACTACAAATCTGGTAGCAATCTGCTAACTGATATTGGAA | |
| GGGAAAGCCCCAGAGACACTACTTGGAAGAATTCCAGTGATTCTTCATGAGCTAAACTAAGACTTCAACCTAAGATAGATAAACTAAGG | |
| AATCACAATCCAACAAAGCGACAAACTGAAATTTTTCGGCTGCTACTCAGCTGAAGTTTGGCAACTGGTTAAACATAAAACACAAGACA | |
| CAGACAGCAATCCAAGATGAAAAGACGATTTTGCTGCACTTTTTTAGAAGCCAGCATTTGTATTTCCATTTCCAGGACGTGGAGTACCT | |
| TTGAGAAGAAAGGTGATTAGTTCAGTTTTAAGCACATTGAGCTGTAACGGTATTATGAAACAGGAAGATACCCTACAAACAGCTGGAAT | |
| TTTGCACATGCTTAAAAGAAAAATGAGAACATGAGATTTCAATTTAGCCATCATCTGCATGGAGTAGACAATTCAATCCAGGAGACTTG | |
| ATAAGAATGTCAAGGGAGTGATCACCATAAAGGTGAGAAATGGACTCAGCATCGATCTCTGCTCAAACATCTCTTTGAGCAGCAACAAG | |
| AGTGCCTTAGCAACCAGCCAGTCAAATCCGGACTTGGGGGAGACTTTCAAGCAAAGGGAATGGTAACATATCCCATTCCCAAAGACCTC | |
| AAGTAGGGTTGAGGAATAATAAATGGCTAGTGGGTTTGGAAACATGGAATCTGTCATGACCTTTACACACTTTTTCATTTGATGGATCG | |
| GGCGATTACTTACATTTGCAAAGGGCCTCAGATTAAGTGGTAATGGTTCAGCATTAGTAATTAGAGATTTTCTTATGTACTTTGTAGGG | |
| AGAAATAGAATGGCATTCAAACAGGTTTAAGTTCCTCTTAAGAACCTTTCTCATTGGATTGTAGAGAAGAGCAAATGAAAGAATCGTTC | |
| AGTATTTCTTGGTATCATGCCCAGCCCATAGGTTTCAATTAATGTTAACCTTTACAGTTGTCATCATCATTATCCAGAACGGGCTTTTT | |
| TTTAGAATAAGCAACAAAACCACTGCTCATCTGTAGACAAGGGAAAAAATCCTTTGGAAGAGAGAAAGAGAAGGGTAATTCAAGAAGAT | |
| CAGGAATAAACATTAAGGGTAAAAACAGAATGGTTAGCCCTGGAAAAGGGAACAAACATGTTTCTCTGAATTAGGAGAGGATATGTGAA | |
| GTAACAGGGATGTTTTGACCCTCACTAACAGTAAGGACAAGAGAGAGTGAAGGAACTTGCACGCAGTGACTTCTACCTTCTCATTAAAG | |
| TCAAGAGAATCTCAACTGTTCAGATATCTGCTTAAGGTAGGAAGAGAGAAATAAGAATTGCTGTCATTTATTTTGTGGTTTTGATATAG | |
| CAAAAACTGTACTAGACACTTTACCTCCTTTAATCCTCACAAAACTTACACATTATATATTTGTATTCACATTCTACAAATCACAAAGC | |
| AAGTAAAAAAGTTAAGTAACTCTCCCAGAGTCCCCCAGATGGTCATGGGAGCCCAGATTCAGAGCCAGCTATGTCTTACTCCAAAGCCT | |
| GTGCTCTTTATTGAAACATACTCCACTAGTTTGAGATCTTGGAGGTGTACCCCTGCACTACTATCACGACTACCCCTCATAAAGTCCTG | |
| TGGCGTACTTCTTCCTATCCATCACCACAGTGAATATCAAAGAAGGGCCCTCTCATTTGAGGAATAACAGACAGTGAAATTGAGATGGA | |
| CCAGAAATGCAAATCCTGGATCCCTGAGGGAAACAACAAAATGAAGCATAGAGGAAATATCTGAGTCTCGTGCCAAGACTCATCAGTAC | |
| AAGAGAGAATGCCTAATGGGCAATAGCAGTTAGGGTGGCAAAAGAGAAGTACAACGGGGTGTCTTGGAATTTGAGGGAAGAACTGCTGC | |
| CTTAGTTTTCGAAGGATGATTATTCGAGAGTTGCCCTCCCTTCACTTTCTCACAGCTGGCAGAATTCCTACCTCTTAGTGGTTAGATTC | |
| CTTTTTACCAGATACTGTAGGGCCAGTGCAGCAACCAGAGCCCCCAGCCAGAAACCCTGCCTCCAATTTAGACCAGTGTTCTGCTCTGG | |
| TGTGTAATAATCATATTCTAATTTAAATAGCCTATTGAGATGCTACATTGATATCTAGCCTTTTTTTTTTTTTTTTTTACTTTTAAGTT | |
| TTATGTGCAGGATGTGCAGGTTTGTTACATAGGTAGACGTGTCATGGGGGTTTGTTGTACTGATTATTTCATCACTCAGGTATTAAGCC | |
| TAATATCTGTTAGTTCTTTTTCCTGGTCCTGTCTGTCCTCCCACCCTCCACCCTTTCATACGCCCCAGTGTGTGTTGTTTCCCTCTATG | |
| TGGCAATGTGTTCTCATCATTTAGCTCCCACTTATAAGTGAGAATATGCAGTATCTGTAATAATGGGTTTTCTGTTCTCGTGTTAGTTT | |
| GCTAAGGATAATGACCTCCAGCTCCATCCATGTCCCTGCAAAGGACATGATCTTGTTCTTTTTCATTTCTGCATAGTATTCAATTGCCC | |
| ATTTTTTAAAAGACAAAATAACAAAAAAAAGAGTACAGTTATCTCCACTCCTCACCCACTACCTGCCACCTAACCCAACCCTTTATATT | |
| TTAGTAATTGCCTTCCATAAATAACATTTTATCCTGTCTTAATTATGCATTCATTAGTGTATTAATTATTGAATGCCCACAATATGACA | |
| AGCATTATTTCAAGCACTGAGGATACAGCAGAGAATAAATCAATATCCTCACTGTTTTTATTCCATTTTTTTAAGAGACAGGGTCTCAC | |
| CCTCTTCCCCACGCTCGAGTGCAGTGGTGCGATCATAGCCCAATGCCACCTTGAACTTCTGGGCTCAAAGGACACTCCTACCTCAGTCT | |
| CCCAAGTAGCCGGAACTACAGGTGCACACTACCACACCCAGCTTCCCCACTCTGAGCCTCCATCCTAATGGAGAAGAGAGGCAGTAATC | |
| AAAATACATGAAATGTGTAGTTATTAGATGGTTATTAATGTATAGAGAAAAATTAACCAGAGAAACGAGCAACGCATTAGGGGGTCAAA | |
| GTTTTGGTAGGGGTAGAGTGGTCAGTGAAGGCCACCTTGAGAGATGGCAATTATGTAAAGCCTGAAAGCAGGTAAGAAATATACAGGTA | |
| TACAGATGTCCCTGGAAAGAGTGTTCTAGGCAGAAGAAATAGAAAGTGCAAAACCTAAGATTGCTCACCTGGCAAGTTCAAGAACAGTG | |
| ACAAACTGATGTGGCTGCATAGGACTAAGAAGAAGGACCGAGAGGACATGAGATGGTCAGATAGCCTAGAAAAGCCTTGTAAGCCATTA | |
| TAACGATTTTGTCTTTTAATATAGAGAAATATTAAAGGTTTTTGTTTTAACTTTTTAACATAGAAAAATGTAATCATATACAAACACAC | |
| AATAGTATAATGAGCCCCCTTGCACTAATCACTCAGCTTTAACAGCTTTCACTTTGGGAGGATTTTGAGTGAACCAGTGATAACACATG | |
| GCATGCCATGACATGCCTTCAACAGGATCACTCAGGAAAATAAGCCATGGGGAGACAGGACAAGCAGAGGAAACACAGGAGCAAAAGCT | |
| TAGGAGACAGATGATGGTGGCTGGAAACCAAGGGAGTAGCAGTGAAGGTGGTGAGAAGTGATCAGATAGAGGATATTTTTTTGAAGATT | |
| TGAATTAATATTATATATATTATACTTTAAAGAAGAATTTGAAAATCCATGTAGTGTAGTAAAGATGGTAAGAAATACAGAAAAGAGGC | |
| CAGGCACAGTGGCTCATGCCAGTAATCCTAGCACTTTAGGAGGCTTAGGTGGGAAGCTCCCTTGAGGCCATGAGTTCAAGGCCAGCCCG | |
| GGCAACATAGTGAGACTCTGTCTATATGGAAAAAAAAATTAACCAGGTGTGGTGGCATGCACCTCTAGTCCTAGCTACCTAGGAGGCTG | |
| AGGCAGCAACATTCCCTGAGCCAGGAGGTTGAGTCTTCAGTGAGCCATGATCATACCACTGTACTCCAGCCTGAATGGCAGAGCAAGAC | |
| CCTGTCCCTTTAAAAAAAAAAAAGAAAGAAAAAAGAAAAAGAAATATAAGAAATGGCACTAGAATATCAGTATTATATACATTATTTTC | |
| TGTTTAAAAATCCACACAACTGGCAAAAAAAAGTGACATACATGAACATTTATAACACAGTGCATTTATAAATTACAAACTCTCTGCTC | |
| CTCCCATATTATTTTGCAGTGATGAATGATTAGGCCATCCAAACGTAAAGATTTATTTGTAATTTGCTAATGATGGAATGACACCTTTT | |
| GAGGTACAAGTTCCCAGCAATTCTTGGAATTAGCAATAGTGTGTTACTTCTTTCTCGAAAGAAGCATGATTTGTAAGCACCTATATCTG | |
| ATGTAGTAAAACTGAAAGTTAAAATAAAAAAGGAAACCGTGGGTCAAAGTATGAGGGAAAAGATAAAACTTTCAGAGCAAAGTTATACA | |
| TAGAGATTTTATTCATCGTCTCTGTGTAACTCACGAGAACTTCTTAGCTTATTTATTATGTCAAATGGCAGTTTGCTCTTCTAATCCCA | |
| GCCTGATATCATGAGCCATATATGCTGTTGGTCATTCAAAAAGGCCACTAAACAACGTGAAAGAATGTCAGTGAATCAATGCAACTCCA | |
| TGAGTACTACCAGGAAAAGAAAAACAAAAAAACATGCCATTAATAGTGGGTCAAAGAGGATTTTTTTCATGCCATCCCCTACCTTACTG | |
| AGAATACCACATTTATATTCAGAAGACTCATAATGAATTCCAGAGTCCTCTGTGTCTTGCCTGTATGACTTTGGGTTATGTCACTTTGT | |
| TAATCTGTTTGAACTTCTGTTTTCTTATCTACAAAATACGCGTAATAGAGTCAACTCTCCCTCCCTCACAGGGACTTTCGGAGGCCCAA | |
| GGGTCTTTACAAATAATAGAGAAGGAAAGAAACTGGCACTTTAATAGTACCTACCATGTACCAGGGACTGCGTAGTCCCCACGGCAGCC | |
| CTCTGAAGTCATGTGATTGGCCCATTGTGTAGATGTAGAGCTTAAGACTCAGAGAGGTTATTTAATCTGTGTAAGGCCACAGAGCTAGG | |
| AGGGGGAAAAGCTGGGATTCGATCACTGACTAGATCAACTTGAAATCTCCTGCCTGACAGAGAAAGAAGAGACTAACTTTACTAAGCAT | |
| CTTTTTATGCCAGAAACTGTGTTGGACATAATTCTGTACTATCTTATTTGGTAAAGGTCATTCTGTCCTTCCTGGTTGCCCTGGTTGCT | |
| CAACAAGTAATAAACAATAAAGCCTGACTGTTTTTCATCCCTGAGACAAGAAATATTATTAAAATAGCGTTATTCTCTTTCTTTCTTTC | |
| CTTTTTTTTTTTTTTGTGTGTGTGTGTGTGTGCGCCTGATAATCTGACTTCCAACCTCACAGAAAACAATGAATGCATGGTTTCATGTC | |
| TGTGGCATGACTCTGTGAAGGAGCCTTGCAAGGGGACAGATACACCTCTACCACTTCTTATAGCCATTCCTGAGACCTCTCTTCTCACA | |
| ACACATACCAGCAAAGCCAAACAAACAAGGTGTTTAGGAATAACTGTCATCTATCAACACTCTCTAGGCAAAGTAGAACTATGAAACAC | |
| TGACCACACTCCACACACACACAACAAAAAATCATCATGTCACATGGTAGCACAGAGCAATGGGAAATGTGAATAGTTTGTTGAGGTGG | |
| ACAGACCCAAGCTCAACGATTGCCTGATCGCTCCATTTATAGGAACCATGGCAACCTACCTTATCTTACTGACCCTCAGTTCCTCCAGC | |
| TGTGAATCGGGTACAACCTCTGACCCCACAGGGCTGTTGTGCAACCATTATCTCAGTTAGCATGTTCCATAAACACAGTAAGTTAGAAA | |
| GTATAGCTGTATAGAACTCTGGGAGTTTAACAAGCAAGAGAATTTGACAGTAATCATTTAGTTACTGAACCCTGTCTTCATATTCCGCT | |
| AGGTTGCATATGGTTTTCCTGCCTGTCCCTTCCAATCACATTTCACAAAAACCAAACCAGTAGTGGACCCTTGTGGGCTAAAGTGGTGA | |
| GGAGGGTGTCATGGAACATTCAGGTTCAGGTCTTCCTCTTGAAGAATGGCTGCTTCTCAATAGGGAACTGGAGCAACAACCTGGCAGTT | |
| CTCACCTTGAAGAAAATCCTGAGACGTGATAATCCGTAGGTCCAAGCACGTCCTGAGTAGGACTCTGAGCACACACCCTTTCTTTGCAT | |
| GTGCTGTTTTTCCTGCCAGCAACACCCTCTAAACCCTTTGCCCTCACCCACCCCCTTGCCTGGTTATACTCCTAGCCTTCTTTCAGATC | |
| TCCACCCTCACTACTCCCTCAGCAAGCTTTCCCTGACCTCCTGACCCAGTCAAGTGCTTCTTATATATCTCCTTTCTAAGCTCTTCTCA | |
| GTTACACTTTTACATTTATTTGTCAGATTATTTGATTAGTCTCAGTCCCACTCTTTAATTCTCCACAAGAACACAGATTGTGTCTGTTC | |
| TCCATTCACCATGCATCGCTTCATCCCCTAGTTTACTGCAGTCTCTGGAACATAGGACGGGCTTTAAATACTCGCTCAATAAATAGAGA | |
| AATGTTGGTTATTAGTCACGTATTACCTAGGTGGGAGAGAGGATTTATATTTGCGATTCCTAATCCCATTAAGTGCCAAGCACTGCATT | |
| AACTCATTTAATCCTAAAAAAAACTCCTATGAAATCAAGTGTATTATTCCTTTTAACTTTAAACACAGACAAACTGAAATCCAGAGATG | |
| TTAACCATCTTTCCCCGTTCACACAGCTAAGTGGTAACAGTCGGTCTTGAGCCGGGCAGCCTGGCTTCAGACTTCCTACACTTTACCAC | |
| ATTGTTCTGCTCCTTAGTGCAGTAGAGGACAAAGTGAGGTGCACACGTGAACTCAGGACAGATTTCCGTTTGAAAATTGTGTATTTTCC | |
| CCTCCTCTTCTTCATTACCATTCTCATCATCATCATCATCAATCACTTCACAAGCAAAGCTAAGCAGAAAAGAACTACTTCATGTATCA | |
| CCTCTTACATTCATGTTAACTCTACATTCTGCCAAAGTGATTTGGCTGAATTTACCCAAAGAGCAAAGTGGACATTTCAAATCCTGATT | |
| CCTAGAAAATTATACACTACATCCTGTAACCACCTTTGAAGAACCACCATGTACTTTACCCCAACCTGACAAAATTGTTTTAAATAAAA | |
| AGAGGTAATTTCACTAAGATTCGTATGATCTGAGACCCATGGTTTCTACATCTTATTCAAAAAGCAGATCCAGCATGTGGTCATCAGTA | |
| GGTATTGAGTCATAAAGCTCCTGGAACAAAATCTTAAGTTTCATCAAGAATGCCAAGAAGTCCTGGGTGTCTCCAAGAATGATAGCTGT | |
| TACTATAATAACATAATTACTACTGCAAAATTACCAAAGCCCAATAAAATATGGCATGCAAAAAAATATTTACCCTGGAAATACAACTG | |
| ACCTAAATCAGTCGTTTATTCCAGCCACCTGCTTTTAGATAGTACTTAAAAAAAAAACTTTCAGGATGGTCATATTGTTTAAAGTTCTC | |
| TAGGTGGAAGAGTACTTTACAGTAATAAATTAATAAATAAGACAAGTTAATGCTTTTAAACTACTATTATTTGTTGAGCATTGTTGTAA | |
| AAGCTTTACATGCATTAATGTATTTAGTCCTCACAACAACCCTTTGAGATACTATTATCATCATCTCCCTTTTACAGATGGGGAAACTG | |
| AGCAGTGCCCAAAGTCACACAACCAGGAAATGGGGAACTATCTTCAAGCCCAGCTGGTTTGCGCCTAAAGTCCATGCTCTTACAGTGTC | |
| CCTGGCTAGCTGAGTACTCAGTTTTGTCACCTTTGTCTCCTTGACTTTAACTGAAAGCTTAACTCTTTTTTTTTTTTTTTTTTTTTAAA | |
| CGGAGTCTTGCTCTGTCACCCAGGCTGGAGGGGCAGTGTCTTGATCTTGGCTCACTGCAACCTCCACCTCCTGGGTTCAAGTGATTCTC | |
| CTGCCGCCTGCCGAATAGCTGGGATTACAGGTCCACACCACCATGCCCGGCTAATATTTTGTATTTTTAGTAAAGACAGGGCTTCACCA | |
| TGTTGGCCAGGCTGGTCTCAAACTCCTGACCTCAGGTGATCTACCCGTCTCGGCCTCCCAAAGTGCTGGGATTACAGGCATGAGCCACC | |
| ACGCCCGGCCCGAAAGCTTAACTCTTAATAAGAGCCTGCAATGTTCAGACGCTTTGAAAAAAACCTAACACACACACTTGCTCTCTATA | |
| AACTTAATTTTCAAAGGAACCACTATTCAAAGAAGTAAAAGGGAGACTGAATAAAAAGAACTAAGTTCTCCGTAGAGCAAATGAATTTC | |
| TGAATCGATTGCAAACAGTTCTCCGTCATCATGACATCCTATGAAGTATGGTCTCCCCCATGTTAGAGTGCCAGTGTCTTCATGTACCA | |
| TATAACTTGCAAATAGTATCACTGTCTTCTCACACTTCAGCTTTCTTCTTTCATCAGTATTTTGGAAGTCCACAAAAACCCAGGGGGCT | |
| CCCTGTCTGTTGCCCTGTGGTCTCTGTGAAAACCAGATGATGCGATGCTGACTTGGACTCTTTCAAATGGATCCAACAGCACATGAGTC | |
| ACTGGCAGCATGTTCAGCTCCTTTACCTCAGGAATTACCCAGTTCCTCATTCTTGCCACCCTTCACCCGTCAAGTAGTAATATGAAAAA | |
| TACATGAGTACTTTTTGACGATACCTCGGACCTCTTTCCTTCAGTACCAGAGACTAATTGCCTCATAATGGTAACATTGAAGAGCCCAG | |
| TTATCTGACGTGATTGGGCTTTAATTATGGATTCCTTCTGGTGTCCTATTAAATTGGAAAGCCTACCTGATTTCACCCAAATGAAGCAT | |
| TACCTCATGCCTTGCTCTCTTGCATTCCTGTGCTGGTGTCACTTAAAAGTCTAATTACATGGTTTATTTCGTGTTCTTTCACTCTTGAT | |
| TTATTGCTGAAGAAGCCCCTCTGGGATATATCATCATAACAAAGACTGATTTCAGATGTCAACACCAAGAAACACAACTGGCATCAGCA | |
| ATCTATAGAGAGTATCTAGGGACAGCAGTCAGCTTTGGAGAAAACCCAGAACTTCTCATTCCCCATCCCACATTTAAGGGCAAAGACTT | |
| TCTCCTTGTTAAAGGGAAAGCACTGAGTTCATAATTAGCTTTATACCCAGAGTTTTTTTTTTTTAATAATTATACCTGTTTAAGAAATT | |
| TTTTGTGCTTTGAGGTGGTCAAGATCAAATTTAATTAGCAACCCTCTTCGTGATTGGCTTGCTCTGGGGACCCTGCTCTTTGAGAAGGA | |
| GGTTAGAAGGTTTAGAAGCAAACCTTTGCAGCAGTTCTAGCCAAGTGCATGCATACAGGCCTTGAGGTGGAGCTGCACACTTGACTGTA | |
| GGGGAGTCTGTCCTTTGTTGACGAGTGAAACTTCTCTCCCTGCAAACCCTCTGGGGAATAATGCCAACTCTGATCAGCTTTTCGACTCT | |
| TGGATGAAATATTGGGTTGGGAAAAAGTCCCTACTAAGAAAATTGTACCAAAACCCTGCTTCAATAAAATATGAATCCATTTAATGAAG | |
| TTTATAGTAGAAGTAGCCTAGTCGAAAAGGATTCTGTCTCTTTCCAAGTATTATTGGCACGTTTAGGGTCTTCCTCAAGTTTATATCAT | |
| TAGGACTTCCATCAAAGAGTTATCTTTACGTGTGATTCCCATCCTCCCTCCCTCTCTCCCTCCCTCCCTCCGTCCCATCGTTCCTTCCT | |
| CCCTTTCCTCCTTCCTTTTCTACCTTTCTTTTTCTCTCTGTCTCTCTTTCTTTCAAGCTGTGGGCTGCTTGTACAGATTTATGCATTTT | |
| GAATTACTGCATAGTTCAGAGATTTGGCGCTTAGGTTATTCAGTTCTTCAAAAAGTACAGTATGACCTAAGGAGATAGGGTGACCAACT | |
| AATCTAGGTTTTGGCTCCAAAAACCCTGTGTCCCAGAAAACCCCTCAGTCCTAGGCAAACCAGGATAGTAGTTCATCTTGTGAGGAGAT | |
| TATAGTGCATTTCCAAGTATTTAGCTGAATTTATCACAGGGCTCTGACTTGATAGCATTTAACAGCGTTAAAATAAGGTCTTTTCTGTT | |
| ATGTTAGTGTGACTTTAGTCTTGGCACTTTCTCAAQGAAGGAATCCAGTCTACTACAGGGGCTAGGTGACTAAGGAGAAGAAAGAGGAT | |
| ATCTAAGTGTTTTTTGCTAAAAATGTCATTATCTCCATGCTATTTCCTTTTTATTCCTATTGTCATCATTTTAAAGACATACCAATAAG | |
| AAATAAATTACTTTAAATAGAAAACTCTAATTTTATCAAGCAACAGCAAACAGGGGTGAAATCTAAGCATTTGTACTTAAGGAGTACAC | |
| TTTTTTGAATTGAAGAAACTTACTATGACCTGAGCATATGTAACTCAATTATCAGAAGTCAGAGAGGGCTACTGAAGGCTCAGGTTTTC | |
| TAAGAGATGACTGGAGGCCAAGTCCTCAGGCTTCAGTCTTTATAGCCAAACTTCAGAACCAGGTCCCTGGCAAGATTCTCCTGTCTAAT | |
| CACATGGCACAGAGTTAGGATGGACTGTTTCTGCTTACCTGCCTTCTTCCTTTGATTTCCATCAACCACCAGTTTCTTTCTCATCAATC | |
| ATTTACCCAGCTTTTGTGACCAAGAATTTTGATGAGGATGCAGAGGAAGGCATGTTCTTGGTTAATAGGAAAGGAGGCTTCATGACAAC | |
| ACAGCATCCTCTAAGATGTGTTGCTCCAACTTTCACAATACATATTGTGTTTTAAGTGGCCTGCTTCTTGGAGTGTATCTTCCCACTAC | |
| ACAAAACAACCACCATTAACATTTTCATTTACTTCCACCCTGTCATTTGTTCATCACATACTTTTAAAAACAGGGCTGTAATCATGTAT | |
| ATACATATATGAATAACTTTATATTGTACTTTTTATACTTACTGGCTTATCATAAACATGTTTCCATGTTGCTGGAGAGTCACATATGC | |
| TAACTTTTGGAATGTGCTTCTTATATAGATCTGCAACTATGTGGGACCAGCAAAGGTTATTGTTCAGTTGGTCACAAATGGAAAAAATA | |
| TCCACCTGCATGCCCACAGCCTGGTGGGAAAACACTGTGAGGATGGGATCTGCACTGTAACTGCTGGACCCAAGGACATGGTGGTCGCG | |
| TAAGTAGGGGTATATGATGCTGTGGAAGCTAGGAACAGATAGAATATAAGGATGCAGGAACTTAGATGAACACGCCCCTTTCATTAGGG | |
| ACCTTTCTGAGACCCTCACATGACCTCAAAAAACTGTGTAAACTTGTGCTTTCATTTGGACTCCAGGAAGTAAGGTGACCACATGTCTG | |
| GATTTGCTTGAGACAGCCCAGTTTGCCCCTGACCTAATTGTTTATAGCACCCTCTTCTACTCTTAAAGGTGGCCCAATTTGGATGATCA | |
| CTTATATGTGGTCACTTGACCTACAAGGTATTTGTGCCACTATTAATTTTTTTTCTCCTAGAAATAAAAATAAGGACAAATTTAAAGAA | |
| ACATACTATACAACACAATCATCACTTTATGTATATTTTTACATTCTTGATGATTTCTTCAGTGGATTTATGGTGTGAATATGTAGGGC | |
| TTTAAGTGAATGCATAAATATCATTTCAGCTCTCTGCTGCTTAGAAAGTAATGCACAGATAAGAAGCCAAGGAATGCAAATTTATTTTG | |
| GCATTCATTACGCTTCAAATGTAAATAAAAAATGACGTATTCATTTAACGGTTGATCAAATAAAACAACCAATTTTTAATGAATCAACT | |
| TTTATAGAAGTAATTTCAATCCAAAAGAATTAAGCTTTTAAATAAGACATATTTATTGTGCTAAAAAACTTGTATTTTCATCTTCCTAA | |
| TACACTAAAATGGCACACAGAATAATAGTGTAGCACACAATACAAACTCATATCTGCAATCAATAATTCTAAAAATTGTTGTTTTAAGT | |
| CTTTCTTTGGAAGTCCTTAAATGCAAAAGAACAAATTTTTAATTTTGTTTAATTTTTAAAAACATTTTTCAGGGTTTTCTAATCCTGGT | |
| TTAAATAGTTTATGCTGAAATCCCAGTTAATCTGGCTTATTCACTCAAAAGAAAGGAATTGTTTAAATCAACTATAATTAAGCTGATGG | |
| TTGCATTTCACGCTTTGAATTCCGATCAAGTTTCAAATTGGTTATTATTTTGGAGGTTCTCCTTTCATGTTCCTAGCTATTTCTTTCTT | |
| TGACTGTGAAAATATTTAAGTCTGAAAAACCTAGAGAGAGAGCTTTCTTTTAAGTCACTAAAATCGCATTCTTTTCATGTTAGAAAAAA | |
| AAAACACTCTAGATTGAACCAACAACCTTAAATTGATTTCAAAAATTAAAGTGTGCTTTATATGAACTTCTTTCAACATCCTCTTGAAA | |
| GACTCCCCTGTCTCCTGTTGTATGTCACAGTTTGTTCATCATGTCTTGTGGAATACCCTTCCAGTTCCTGATTGACCTAGGAGAGCTTT | |
| GAAATGCAGGCTAATTATCACAGTTTTACACTCATTGATCACTTAGTGTTTCTCCAGATTTATTCCAGTACGGTTCTGTCAGTTTGAAG | |
| TGTTTATTTTCTTTGCTCTTCTTTAATCCCTATTATAATCATATCCTTTAGACCAAATTTTAGATGAACTTTTCTACTACTATTTATTA | |
| TTATAAAACCTCCAGTATCGTGTCTATAATTTGGTGTTTCTGTGGTTTGCTTTAGTAATCTTTCAGGATTTTAGAAAGAACTAATGGTT | |
| ATTTAAATTTGAGACCTTAAAGCCGCAATACAACTAAATTGTTTCTAACCAAAGTGACCGATTCTTTCACTCTTGTAGTGCAGTGGGGT | |
| GTGGCCTCACTACTTCCGACAACTCATCAGTCATTCACTCATTCAACAAACATTTTGAGCATCTCAGCTCCTGAATTTCATCATTATGA | |
| TGTCTGGTTGTAGAGCAGTGAGATTTTCTAACCCTATATAATTTCAGTGAGAAATTTAATCAAGATGCCACTTGAGAGTTACAATAGAC | |
| TCAGTAGTCTTAACGTTTTTAGTGTTTCCTATGAAAAGTCAGTACTCACTTTCTAATTTGATTTTTACCTGAGAAATTTTTGCTTACAA | |
| TGTTTCCCAAAATACACTATTTCTTTCATAATTCATTTGTTTCTATCTCTCTCTATATATACATTTTTAAGATTTTCAATGTCAAAACT | |
| TATTCCATTTATGTACAAACAACATCGTCCTACCATTCTCCAAACCAGTATTTTATTCCCACTACTGTTCCTCTCAACAGACGACTGGG | |
| GGCAAGTTACATGACTTCTTGGCCACCTTAATGTCTTCCCTCATAAAATCAGACAGTTTCACTACAGAATCTTATCTTCAGCCTGTTTA | |
| CATCTCCAGAATTCTGGACTGAACTAACATTACGTAATATTATGAAATAACATGAGCAAACTTGCCACACACATATTTATGGTATGACA | |
| TTGATTATTAGTATTTAGTTCTGACTTGTAGATAAGAGTATCACATAGAGAAACTTACCAAGATAGCCAAAATTATGTCTTAGACTTGT | |
| CAATACTAGAAATGAAGCTAAGATAACTTGTTTTCAATAATACAGATTAGGCAAAGAGTATAGATTCACAAGCAAGACACTAATCCAAC | |
| ATACCAAAAAAGATATCCAAAAAATGGCAATGAGAAGACAAATAACTTTTAAATTGTCAAAACCAAAAGAATTACTTTCTTTGGAGTGT | |
| TTATCTTCATCTTTCCCTCAGTATGAATTCTTAATAAACTTAGTTTCTACTTTCAAAAGGATTTTATCTATGTTATAAATATTCATGTT | |
| TTAAATAGCCAAGTCTTGATTCTGGTTTGATTGCTGTAATGTAGGGGATTTGTCATTTTAATATTTTTTAAGTTAGCTTTAATTTTGTC | |
| TTTTTTAAAATTCATTTATATAATTAGCCTTTACTGCCTTTCTTGGTAAAAAATAATAATAATAATCTTAGTTTCGTCTTTTATTTTAA | |
| ACCCTGAATATCAACAATTTGTTTCAACATATTTTAGTTCGTTGACATTTTTATTCCATGGAAAATATCTGTGTTCCTTGACTTTGCTT | |
| TAGGCCAGTTCACTTACCAGTAATCACCACACTCTCTCTGCTTTCTGGCACTTTACAGACATGGAATTTTTTTCCAATTAATACATATA | |
| GCATATACCAACTGGTAAGCACTAGGAGTAGATATATAAATATCAAAATACAGAGGAGAAACAAGGCCTTCTTCAQTGTCTGTTGTTCC | |
| CCTTTTTGTCCATGAGTTCTCATCATTTAGCTCCCACTTACAAGTGAGAACATGCAGTATTTGTTTTTCTGTTCCTGCATTAGTTTGCT | |
| AAGGACAGTGGCCTCTAGCTCCATCCATGTTCCTGCAAAACATATGATCTTATTCTTTTTTATGGCTGCATAGTATTCCATGGTATATA | |
| TGTACCACATTTTCTTTATTTATTTATGTTATTGATAGGGAATTTAGGTTGATTCCATGTCTTTGCTATTGTGGATACTGCCGCAGTGA | |
| ACATTAACATGCATGTGTCTTTATGGTAGAATGATTTATATTCCTTTGGTTGTCTACCCAACAGTGCGATTGCTGCCTCAAATGGTAGT | |
| TCTATTTTTAGCTCTTTGAAAAATCACCACACTGCTACAAAATCAATGTGCAAAAATCACAAGCATTCCTATACACCAATAACACACAA | |
| ACAGAGAGCCAAATCATGAGTGAACTCCCATTCACAATTGCTTCAAAGAGAATAAAATACCTACGAATCCAACTTACAGGGGATCTGAA | |
| GGACCTCTTCAAGGAGAACTACAAACCCCTCCTTAATGAAATAAAACAGCACACAAACTAATGCAAGAACATTCCATGCTCATGGATAG | |
| GAAGAATCAATATCGTGAAAATGGCCATACTGCCCAAGGTAATTTATAGATTCAATGCCATCCCCATCAAGCTACCAATGACTTTCTTC | |
| ACAGAATTGGAAAAAACTACTTTCAAGTTCATATGGAACCAAAAAAGAGCCTGCATTGCCAAGACAATCCTAAGCCAAAAGAACAAAGC | |
| TGGAGGCCTCACACTACCTGACTTCAAACTATACTACAAGGCTACACTAACCAAAACAGCATGGTACTGGTACCAAAACAGAGATATAG | |
| ACCAATGGAACAGAACAGAGCCCTCAGAAATAATACCGTACATCTACAACTATCTGATCTTTGACAAACCTGACAAAAACAAGAAATGG | |
| CGAAAGCATTCCTATTTAATAAATCCTCCTGGGAAAACTGGCTAGCCATATGTACAAAGCTCAAACTGCATCCCTTCCTTACACCTTAT | |
| ACAAAAATTAATTCAAGATGGATTAAACACTTAAATGTTATATCTAAAACCATAAAAACCCTAGAAGAACACCTAGGCAATACCATTCA | |
| GCACATGGGCATGGGCAAAGACTTCATGACTAAAACACCAAAAGCAATGGCAACCAAAGCCAAAATTGACAAATGGGATCTAATTAAAC | |
| TAAAGACCTTCTCCCCAGCAAAAGAAACTACCATCAGAGTGAACAGGCAACCTACACAATGGGACAAAATTTTTACAATGTATCCATCT | |
| GACAAAGGGCTAATATCCAGAATCTATAAAGAACTTAAATAAATTTACAAGAAAAAATCAAACAACCCCATCAAAAAGTGGGCAAAGGA | |
| TATGAACAGACACTTCTCAAAAGAAGACATTTATGCAGCCAAAAGACACATGAAAAAATGCTCTCATTATCACTGGCCATCAGAGAAAT | |
| GCCAATCAAAACCACAATGAGATACCATCTCACACCAGTTAGAATGGCAATCATTAAAAAGTCGAAACAACAGGTGCTGGAGAGGTTGT | |
| GGAGAAATAGGAACACTTTTACACTGTTGGTGGGACTGTAAACTAGTTCAGCCATTGTGGAAGACAGTGTGGCGATTCCTCAAGGATCT | |
| AGAACTACAAATGCCATTTGACCCACCCATCCCATTACTGGGTATATACCCAAAGGTTTATAAATCATGCTGCTATAAAGACACATGCA | |
| CACTTATGTTTATTGCAGCATTATTCACAATAGCAAAGACTTGGAACCAACCCAAATGTCCATCAGTGATAAACTGGATTAAGAAAATG | |
| TGGCATATATACATCATGGAATACTATGCAGCCATAAGAAAGGATGAGCTCATGTCCTTTCTAGGCACGTGGGTGAAGCTGCAAACCAT | |
| CATTCTGAGCAAACTATCGCAACCACAGAAAACCAAACACTCCATGTTCTCACTCATACGTGGAAATTGAACAATGAGAACACTTGGAC | |
| ACAGGGTGGGGAATATCACACCCCTGGGCCTGTGGTGGGGTGGGGGGAGGGATAGCATTAGGAGATATACCTAATGTAAATGCTGAGTT | |
| ACTCCGTGCAGCACACCAACATGGCACGTGTATACATATGTAACAAACCTGCACATTGTGCACATGTACCCTAGAACTTAAAGTATAAT | |
| AAAAAATAAGCAAGTTTACAAGGGCAAAAATAAAACAAAAAAGAAAAAGCACCACACTGCTTCCACAGTGGCTGAACTAATTTGCACTC | |
| CCACCAGCAGTATATAAGTGTACCCTCTTCTCCACAGCCGTGCCACCATCTGTTATCTTTTGACTTTTTAATAAAACCCATTCTGACAG | |
| GTGTGAGATCATATCTCATTGTGTTTTAATTTGCGTTTCTCTACTCACCTTTTTCCATATGTTTGTTGGTGGCATCTGTGTCTTCTCTT | |
| GAAAAGTATCTAAAACAGTCACTTATCTTTTAAAGAAACTTTTTAAATCAGAAAAAAGGTGTTTATATTTAACCACGTATTTATCATTT | |
| GCAGTGCTGTTCATTCTGTTTCTTAGGTCAAAATTTCTATCTGGTATCATTTTCTTCTGCCTCAGGCACTTCCTTTTATTATACTGCTG | |
| ATCTGATGCTGATTAATTCTTTCAGTAGGTGTATGTTTTCATAQCTTTTTATTTTATCTTTGTTTTTCAAAGATATTTTGAAGAGTATA | |
| GAATTTTACGTAGACACGCCCGGCGCAGTGGCTCACGCCTGAAATCCCACCACTTTGGAAGGTCGAGGCGCGCAGGTCACCTGAGGTCA | |
| GGAGTTCAAGACCAACCTGACCAACATGGAGAAACCCCGTCTCTACTAAAAATACAAAATTAGCCAGGCATGGTGGTCCATCCCTGTAA | |
| TCCCAGCTACTCGAGAGGCCGAGGCAGGAGAATCACTTGAACCTGGGAGGCAGAGGTTGCGGTGAGCCGAGATCGCACAATTGCACTCC | |
| GGCCTGCGCAACAAGAAAGAAACTCCGTCTCAAAAAAAAAGAATTATAGGTTGACACTATTATTCTTTCACATCCTTAAACTATGTTGT | |
| TCCACTGTCTTCTGATTTGCCTTGTTTCCAAGAAGTCACCTGTCAATCTAATCTTTCTTCCTCTGTATATAATTTTTTTTTCTCTCTAG | |
| CAGCTTTTCAGATTTTCTCTTCCTCACTCGTTTTAAGCAATTTGATTATATGGATATTAGCATAGTTTCCTTCATGTTGCTTGTGCTTG | |
| GGGTTCATCGAGATCCTTAGATCTCTGGGTTTATATATTTAGTACGTTTTAAAACTTTTTGGCCATTATTTTTTCAAATATATTTTCTG | |
| TCCACTCCTCTTCATCTTCTTCTGGAACCCCAGTTGCACATATATTTGGCTATGTGAAATTTCCTACAGCTCACTCATCACCTCTTTTT | |
| TAAAAAATCTTTTTTTTCTCTTTCATTTTGGAAAGTTTTATTACTGTGTCTGCACGTTCACCAATATTTTTGTCTGTAGTGTCTAATAT | |
| GTTCTTAATTCCATCTAGTGTATTTTTTCCTCTTACACATTCTAATTTGCAATTTCTATAGATTTCTTTGGGGTCTTTTTTTTTCATAT | |
| CTGCCATGTTACTCCTTAACATACCAATGCTTTCTTCTTTTTCTCATCATATGCAATATATAATACCTATTCAATGTACTTGTCTACAA | |
| ATTCTGTCTTCTGGATCTATTTTTGTTTAGTTTTTGTCCTAATTATGAATTATATTTTTCTATTTCTTTCCATGCCTGTTAGTTTTTTA | |
| TTGGATGACAGGTATTTTGAATTTTGTATCGTTAACTCTTGGATTCCTTTTTTTTTTTTTTTTTTTTGATGTGCTTTTAAATACGTTTG | |
| AGGATTCGGATGAAGTTAAATTTGGGAACAGTTTGATTTTTTTGATTCTTTCTAAACTTACTTTTAAGCTTTATCAGAGAGACCAGAGA | |
| AACCTTTACTCTAGCCTTAATTTGACATCATTGCTAAGGCAGTACCTTTCTGAATTTTCAACCTCATGCTCCATGTATTACAAGATTTC | |
| TTGAACTATTCCAGCCCCATATGTGTTACAATAATTTTTCTGCCTCCACCTCTGTGGTGGTTCTTTTCCCAGCTTTGACATATTTCCTC | |
| ACACACAGCACTCAGCTGAAGACTCCAGTGATCTCTCAGTGTATCTCTGTTTGCGGTTTCTTCCTTTCCGGTACTCTGCTTGTGAGTTT | |
| TAGCCTCCTTGGCTTCCTCCAATATTTAACTGTGTCTCCTCAACCTCAGAGATTGCCACGCTCTGTTGGGGTTCCTCCTTCCTGTGCTG | |
| CAGCCTGGGACTCTTTAGGCATTAAGCCAGACTAATTACAGGGTTCACCTTATTTATTTCCCTTTTCTTAAAGATTACTGTTCTCTCCT | |
| GCCTGTTTTCTACTGTCTAAAAACCATTTCTTCATGTATTTTACATATTTAACGTTCAAACCAGTCTCTTTTACTCTATCCTTACCTCA | |
| AGCAGAAGACTGACTTCTGTACTGTTTCATTTGTTAACCAGAGTAAATCCTTCATTATTCACATAATAAATTAATAAGGATGATGTTTT | |
| TCTCACACCGACTACATTAGGCAATATACATGAAAACCATATTATTGACAGTAAACTGTAAGATGCTACACAGATGTTTATCATTGCTA | |
| TTACAAAGGAGATAAACCCCGTTTTCCTGCAGTTAGGGAATTCTATATGGGAGTAAGGCTGAAAGGGCCAAAAGATATAGGTATTGTTT | |
| CTGAAAAACTGCCTATGCTTCTATGCATATAAGTATGTCATGTTCCATATTTTTCTGTGCTGTATTAATTCATGCATTCCTTTATCAAC | |
| AGATACTTATTAAACACTCATATCTCAGCCATTGTTCTACCCACTACACATCTCTGCCTTCAAGGAGCTTATTTTCTAGTGCTATATTT | |
| TCTGTTCTATGTCTTAGCTATCCACTTTTTTCATCTGCCTCGACACGTGACTTATTCTGTCTCTGGGCCTCTGGTATGAGTGCTCATTT | |
| CATTCTGCCTTATAACTCCTATTTTCTTCCCTACTTTATCTGACCTTCCTACCTTACCTTGTTCATTCTTTCCTTCAATCCACTTCTCA | |
| TCAAATCTCTTTCTTTCCTCTACTAATTTTTTTTTTCTTTCTTTCTTTCTGAGTAAAAGCCAGAGATCTGGCCCCCTGCTTACCTCTCT | |
| GAATTCTTTACTTACTTCTGACAACTTGCTCATTTCACTCCAGCTACATTGACCTCCTTGCCTTTGTTGTGTTTTCAAAACACCGGCGT | |
| GCTCCTACCGCAGGAATTTTGTACTTACTGTTCCTTTTGCCACATTCATCATTCATATCTTCATGTCTTCCCCATCGCTTCCTTCAAGT | |
| TTTTGCTCAGTTGTCATCTTTTTACTGAGTGTCCTTCCTGACCTCTCCCTACTTTAAAATGTTATGCTTTTTTCTACTACCTCTCCTGT | |
| TCCTCATTCCTACCTTATTTTTCTGGATAACACTTATTGCCTTCTAAATTGTATCGTATAATTTACTTCTTTGTTTCTTCTCCGTTGTC | |
| ACACTAGCATATAAATTCAGTGAAGGTAGAGATTCTTTTACTGCTGACAAAAGCATCTAGGACATTTCCTGGCACTGATAAGGATCTGC | |
| ATAAATATTTGTTGAGTGAATGAATCTCCTTGGTAAAGTCCTTTTTTGTTTGCCTGTTATATTTATTGAATAGACTTCTTTGTTTCATG | |
| TACTTTAATTTCAAAAGTGAATGCCTGCAACATTTTATATATTTTCTTATAAATCATTTTCATTGCTTTTTAAGCTTATCACATATTTT | |
| GTTTTATAAATATGTAGCCTTCGTGAGATAAAAGATTCTCCATCCCTGTTTACGTGTATACTTAGATGACAACTCTAATGGTCATAAAT | |
| AATTCCAACCTTATAGATAACTCAGGACAACATGAGATTATAAGTACACTTTAAATCCCATTCAGAAACCCAAGGACAATTCAAAAGGA | |
| AAATAATCATTCCAAATATAATATTCTTCTTATTCTTAAGAAGTTGTTAGTATTTTGAATTTTGAATTTTTAATACAGCTCCCATAGAA | |
| AATACTTTAAAATGAACCCCGTAAGACTTCCTCATTAAGACATATAGACACATATGCTCCCACTCCCGTAATCAAATTTCGAACTCAAA | |
| TAAGCTCTGAAAACCAAAACATTCTTCCAAGTTTATTGCAGACTCATTTGGTAGTAAAAACCAATCTGACCTGATGTGTAGCTGTTTAT | |
| AGTCTTTATTTTTCCATTTGGTGAGTCCAATTATATATTTCGCTGAGGAAATACTAACATGTTTGACTACATTGTGTGCCCCAGACCTG | |
| CTTAGAGTATTATGTAATATGCAGTATAGGCCATATATTGCCTTTTTAACATCAAAAATACCCTAAATTCTGAAATGCATGTAGCCCCA | |
| AGAGATTTGTATAAAGCATTGAGGGCCTGTGTATTTTTTTTTAATTTACAAGTGAAGCCAGATTCCCTAGACATTAAGTGCCTAACTTT | |
| TGGTTGTTGTTGCTGCTGCTGTTACTGTTTTTTCTCCAGCTTCGCAAACCTGGGTATACTTCATGTGACAAAGAAAAAAGTATTTGAAA | |
| CACTGGAAGCACGAATGACAGAGGCGTGTATAAGGGGCTATAATCCTGGACTCTTGGTGCACCCTGACCTTGCCTATTTGCAAGCAGAA | |
| GGTGGAGGGGACCGGCAGCTGGGAGGTAAGCATCATTTTCCTGGCCTTGATCCTCCAAGGGGTCCAGGCTTTGGTTTTCATCTGTATGA | |
| ATTATATGTTCATCTGCATCCCTTCCAGTCTCTACCCCACACTCCTGTCACCTTTTCCTTGCTCAGAAACCTTTGATGGCATCCTGCTA | |
| CTTCTAGGATAAAAACTATAGCTCCATAGCCTGTCATACAAAGCCCTGCTTCTCTGGCCCCAGCAACTTTCAGCCTCATCTCCAGCCCA | |
| CCTTGCATCCTCCTCTCGCCATCTCTGCTCTCTGAATAACTGAGGCATATTTATACCTTTGTATGTGTCTTCCTTTACCAAAAACACCC | |
| CACCGTTTCCATTTCACCCTCTCGAAAACCAATGCAGAAATCAAGACTTTGTCTAAAGAACACCTCTGTTTTACCTTCCCCAACTCACC | |
| TTATTATAAGGCATTTTTCTTTCTTTGGGCTCTGCAGACACATTATAAACTATTATCTTTATCATAAAAATTGTATGATTTTCCATTTT | |
| CTTGCCTTTTCTCAAATTTTTTTTTCAACCCCATTCCCTGTCCCACCACATGCCCCATTTCTACTTTTGTATTTTGTCCTTTACTCTGG | |
| CCGTCCAGTCGTCATCTGATACTCATGTGATTCAAAACCGATTATCAGTTTTCCACTAAATATACTCCATCTTCTGATACCCAGACTCA | |
| AAACCTTCCCATCCTCACTGACTCCCCCTCCTCGCCTGTGCTGCGAGGCTGTGGTCCAGCCATTCCGTGCTCTCTGGCATTCCACACTA | |
| CATTCCATTTTCATTGTTCCACTTATAGTAGTCTTCTCCCTGGTCTCTGTCCCTCCCCACTTCTAATCAAGTTTACATCTTCTAGCCAG | |
| ATAGATATTACTGAAGTTCTGTATCTTGTTGCTGCTCCGTGTCTCCGTTTTGCCTACAAAATCCAAATTCTGTAAGTTGGCTTTTTGGA | |
| TCTCCCAAGAGCTGGCCTCAGGCTGCATTTCCAATCTATTTCCCGTTTTGCCCCTTCACGTCAAAGTCTACTACTTGGTAGGAGCACCA | |
| TCATTTCTCACTTTCCTGCCTTGGCACATTCTTTCTATTTTTGCTCTCTCCTCTTCCTGGAATGCCTTTATCCACTCTCTTATATGAAT | |
| GTGTTCAAATTCTGCCACCTTTTCAATATTCAGAGCAAATACCTCTTCTGCTCCAAAGCCCTCTGAACTCTCTCCTTCTTTGAAATCCC | |
| ACATCACTTCATTTGTAGCTCTCTTGTAGTACTTGTCTCTTGAATCACTACCTTTTATGATATTATACCTTAATTATCACTATATCCAT | |
| CAGCGTGAAAACTCCAGCAGGAGGGAAGGAAACAGATGTCCCTGAGTCATTCAGTTCCTTCAGAAGTTGTTTTTGAGTACCTACTATGT | |
| GCCAGGCTGTCTGCTTGGCACTGTGGAATGAGCTCACTCCCTGTGCCCAAGCAGCTCTTCGAATAATTTGCACACTTCTCAATACAGCT | |
| CCTGCCTAGTAGCGTTATTCAATTCCATTCCATCACATCCTGTGTGCCTTTAACTCTTACTGTAGAGAGGAAACGGAAAGGTGGGGGAA | |
| GGACAGGAGAGGAGGGAGGCGATCTGATACACGGAAGAGAGTTTTTACTGTAATCCGGAGAAAATGCAGGTCTTCTTTGATGCCTTTCA | |
| TATTGGATGACATAAATGAGACTGTTTTTAACACTTTATTAGCAATATGAAGAGTTTCAAAAGAGGAAAAATGCGTTTTTATTCTAAGT | |
| TTACATTATTTGGGCTTTATAAAAGCATGGTCTTTTAAATGTTCACACTTCCCTGGGCATGAATGGACTGTGCTGTATGGCCCTAGATC | |
| GGGAAAAAGAGCTAATCCGCCAAGCAGCTCTGCAGCAGACCAAGGAGATGGACCTCAGCGTGGTGCGGCTCATGTTTACAGCTTTTCTT | |
| CCGGATAGCACTGGCAGCTTCACAAGGCGCCTGCAACCCCTCGTATCACACGCCATCTATGACAGTAGTGAGTACTTCACTTCCAACAG | |
| GGGGCACACCAAGAATAGACTTCCAGCCCTGCCCTGCCATTTACTTGCTAGCTGAGTCCTGGGGAAGGTAACTTAATCACTTTAAGCCT | |
| CAGCTTTATCATCTGTAAAATGTGAATAGGAAAATCTACCTTGAAAGGTTGATGTCCAGGTTAAATGAGGTATTTTAAGAGGGGCTCAA | |
| TACATGTAATTCTTTTTCCTTTGATGTACATTTTTTAGTCTTTTACATGAAATGTCACATCTCATCTTATTTATGAATGTCTTGCTATA | |
| AAGATATATGGTTGATACTTTTAGAAGGGAGAATAATCCCAATTTTCTTTGGTGGTGGGGGGACTCTGGAAAGGAGTTTATAAAAGACA | |
| ACTATTATTCTTACCCATTTTCTTTCCCATATGTTACTACCTTTATCAAGACAGAAACTTGAAGTTAAATGAATGAGTAGCATAGACAG | |
| CATGTGACTTCAAAATCCTATTTCAAGCCGGGTTTGGCCTCATGCCCTGTAGTCTCACCTACTTGGGAGGCTGAGGTGGGAGGATATGG | |
| AGTTTCAGCCCAGCATCACCAACATAATGAGACCCTATCTCAAAAAAAAAAAATATATATATATATATGTATATATATATTAATTTATA | |
| GTTCCTACAACTTTTTAAGAGTTTTACAGTAACCTTCACCATTTACCATGCAACTTTCTTCTTTGATTATCCATAGCATTGATTAGACA | |
| GGTATTTTCAAAAAGCCTGCAAGCATAGTACTCAGACTCATTGAAAGTTTATAGAATTTGGCCTGTGTGGAAAACTCTGTGCTCCAAGT | |
| ACAACAACTAACTGAATTCTCTAATTTAAACAAGTTTGAAGGGAAGTGAAAGGTTATAAAATGATACAGACTCATACCCCAGAATCACC | |
| TTAAAATCCACCTGTTGCACAAAAAAAAAAAGGAACCTTTATCCTATTACCACAAGAAAGTTTTGCCTCTTCACCACACAGAATCCTAA | |
| TTTAGTTTGGGAAAAAGAAACATAACATCCCCATTTTCCTTACCTGTAAAATAAAAGGTAGTAAGTAGTTTAAGAAAACTQTACTTTTC | |
| CTAAAGTTTTTAGGACTCTTGTAAATAGAATAATAGCAGAATCATGGAAACAAATTTGGAATTGAAGTCTTGACCTCATTGAAAGCCCA | |
| GAAATCAATTAGCTTGGTACTTCCCAATACATCAGAAAACCTCTTTCTCTTTTTGTGTCAGTCTCTCTGTCTTACAGCGAGAAACAAAT | |
| ACCTGTTCCTTTCTACCTCATTTGTTTTGGTGATCATAAATGACCCATGCCCAAGAAGTGTTCCGAGAATTTCAGAAGCCATTCTTGTT | |
| TTATTTACTTTGTTAGTGAAAGCATGTATTTAAGCTTTGTTTTGGTCATGTGTGCTAAGGGGAGGGTCCTACCTAAAGGACTGGCTTGT | |
| TCACAACTCAGGATTAATCATGTTATTTGTTGTTTTTCCCCTGTGAACACAAGCCCCCAATGCATCCAACTTGAATTGTAAGAATGGAC | |
| AGGACAGCTGGATGTGTGACTGGAGGGGAGGAAATTTATCTTCTTTGTGACAAAGTTCAGAAAGGTAAATACATTCTGTGATCTCTGAT | |
| CTCAAGAGGTGTGATCTTCACACACTACAGTTCTGAGTGTGTCTGTGAGTCACATTTCAGCACTGGACAAGAAACATCCCTCTGCTGCC | |
| ACAGAAAGTCTTAAAAAGCATTTACGTTTTACCTCTTCCAAATGTAAATTGTCTGTGTTATTTTTTCCTAAGTCAACTAAATCACTTTT | |
| AGATTTCCCATGGAAGTAAAAACAAATAAGAAAACCCTGATAATTATAATCACTGACTTTCAGTATCTTTATACATTAAAATTAATATC | |
| TGTACTTTGTTAAACAAAAATAAATAATACCCCATTCTTGACACACATACAAACACACACACACAATAATGTTTTACATACTATTAAAT | |
| TGTTTTTATTTCTTTTCCAATGCTTTGATATAACACTCAGAGAACTTCTCTGAACACTTCCACAGATCTGATGTTTAAGAAAAGGAAAG | |
| AAAAACAGGGAATACAGCGAAATTCTCAGAGCACATTTCTCTCACAATTTTCCCTTTGAGGAAGGTTCTTGGCGTTTATCTTTCATTAG | |
| ATATATTACAACATTGATACATTAGTAAAACCTGAGGAAAATGTCTTATTTTCTCAAACTCCATTCTTTTTTTAAATAAAATGATAGTT | |
| GCAAAGTCTAGTATAATATCTATTTACTACAGGGTGAGCATTTTCAAAGTAACCAGATACCTAACTGAAAATATAAATAACTAAAATAA | |
| ATCAATAAAAGACATGTGCCTTTTAAAAAAATTATCATATCAGATTAAATTCTCTGAGGTATATAGTCACAAAAAAATCCCATCAGATT | |
| TTAAAGTAAACACATATATACTTTATCCAAAAAAATTTACACCCCACGCATGCTAACTCATGCCTGTAATCCCAGCACTTTGGGAGGCT | |
| GAGGTGGCAGGATTCCTTCAACCCAGGACTTTGACCCTCTAGTCAGCTATTATCATGCTGCTGTACTCCAGCCTACATGACAGAGCCAC | |
| ACCCTGTCTCAAAAAAAAGAAGTTTACACTTTCTCTGCCTACACTAGTGTCACCCTTCTTCCAATCCACTCTGTCCAAGTAATACTCAT | |
| TTCCACATTCCTGGGTGTACATACTGTGAGTAAACACAATCTCAGTACCTTTATTTCTAAATGAGATCCTTACTCATTATCATTACATT | |
| GGAATGGCTTAGTAAGAAATGTCTTGTACTTGGCTTAAAGAATCTCAATAACTAGGTCCAGTGTAGTCCTACCTGAAGGCAAAGGAGAT | |
| TCTAAGATGTTTTATCCATTCCTACTATTTCTAAAATTCTACTTACATACACATAAAAACTTTCAACAAACTCAATAGTGTGGCCCTTT | |
| CTATGAAACGGAAAGTGAAGCCAGGAAGATGCCAACCTAAGTTACATTGTCACATTTCTGTAGAAGTGAATAGACTATTTATAAACATG | |
| CTGAACATGGAGTATTTATAAACACACTCCATGACTAGAGTGTGCAATTTAAGCATTGCATTTTAATTAGAAGGGACCTGATTATTATA | |
| CTATTTTGACCAATTACTCAATATGAACTAACAGACTATTATAGTGACACACTTTAATACTCTGTTAACACCCTCCACTCTGAAATTAT | |
| ACTGTCTATGTTAAAATCCTGGTTCCTCCAAGTACTACCTGTTCAGCCTTGGATAAGTACTTAACCTCTTTGGACCCCAGTTTCTCTTT | |
| TGCAAAGTGAGGATAATAATAGTACCCATCTACATCACAAGGTTGTGGTGAGGATGAAATCAGTTAATATGTGTATATATGAAGCACTT | |
| AGAATAGCATCTGCCATACATTAATAGTAAAACTTATATAAGTTAATTATTTTTATGTACATCTATATATTGAATTGCACTGCCTCGTT | |
| CGATTCTAATATTCTTCCACCATCCTTCGTCTTTCTCAGGATTCCCTTAAGGATGGAGAAGCAGTAAGGTCAAAGATTAAAAGAAAATT | |
| CAAGTTAACTAGGTAAATCTAGTATTCTGTCATTGTCAATCAAAGTTCCGGCGCATTACTCTGGTAAATAATGAAAAAAGTCAGTGATT | |
| TGTGAAGATTTTAAAAGACTGAACCTTTTGATCTTGTTTTTTAAAAGTGTAAATAGATAGTAGGTAGAATATTAAACCAGTTTTATTTT | |
| TCAGCATGTTTATAAATACTATTTACACTATGTGAAATTACACACTTCAATGTGATTGTTTGCAGATGACATCCAGATTCCATTTTATG | |
| AAGACGAAGAAAATGGTGGAGTCTGGGAAGGATTTGGAGATTTTTCCCCCACAGATGTTCATAGACAAGTAAGTGATTTATTATTATTA | |
| TTAATCCTTATTATTTTTAGACATGCGATCTCACTCTGACACCCAGGCTGCAGTGCAGTGGTACAATCACAGCTCACTGTATCCCCCAA | |
| CTGTTGGGCTTGAGGGATCCTCCTGTCTCAACCTACCAAATATCTGGGACTACAGGCATGCTACCATGCCCAGCTAGTTTCTTCAGTTT | |
| TATTTTTTTTGTAGAGATGATGTCTTGCCATCTTGCTCAGGCTCGTCTTGAACTCCTGGACTTAAGCGATTATCCCACATTGGCCTCCC | |
| AAAATGCTGGCATTATAGGCATGATCCATATTACTATCATCATTTTATACTTTCTGCCTTATTGAAATTTAGTACTTAGCTTCCATCTA | |
| TAAAAAAGAAAAATCATAAATATTTAACTTCCATAAAACAGTACTATTTTTAAGACTCATGCAGGTCATCCAAAAAAGTTACCTACTGT | |
| AGAGTTATGGGCAAGGTTCTGTTAGATCGTTTGCTCTGAGATGTTGGCAAGATATTTAATTGCTGCCTAATCCCCTAACCCATAAGACA | |
| GTGCCCCACAGGTCCTTTAATGAAAATGTTTTTAAGTGTTCTACCAAAGTAATTATGATGAGACCTATTTTATGATAGCACCTAATTAT | |
| GATAACACCTTAAAAGGGTGTTCTAGGAGTCATCTTCTAATTGAAGCTGATGTCATCCTGTTAAATGAAGATTTCCCAATGGCAACTGA | |
| TTAAATCATCACTTTAATCATTTAATAACATGATAATGCTGACCCCCCACCTTAATTATTATTCTCCTATTTACAAGTAATTGAAGAGT | |
| ATTCTCCTATTTACAAGTAATTATCTGGATTGGAAACAATCCAGATATTCATCAGTAAGTGGCCAGTTAAATTATGGTCTGTCCTTTTG | |
| ATGAAATATCATGTAGTTATTCAAAAGAATGAGTACTCTGCGAACTAATATAGAAAGATCTCAGTTATACAGTGTTAAAATATTTTAAA | |
| AGATGGGTGTACAACAAAGTGTGTGTAGAAATGATTCTATGTTCATGCATAAAGAAATTCTGGAAGGATACCTAACAAATTAATAACAG | |
| TATTTACCACTGGCTGGTACACTGTAGGAACTGCATGGATGGGGAACAAGAATGTCAAAGAGAATACCACTGTATGCTTTTTTATATTT | |
| TATAAGATCTTTATCCCTGTGAATATATTACATAGTCAAAAAATCAAACTGAAAAATGCTTAAATCCCTAACATACCACCAAGAACTAA | |
| TGTGTTATGATGCCAATGTAAGCAATGTTTAGTTTCTTCTCTTCATCACTGGCCTGTACCCATCTCTCTCTTTCCATACCCAGTGTTTC | |
| TGATGTTGCTCTAAATAATCTTGGAGGCTTTTAAGGCTGCTTTGGAAGAGAAGAAAGTATATGCAGTGAATTTTATAAGCATGATATTT | |
| ACAACTAGAAGACATTTGAAATAGACAAATGAAATAATAAAGCTTCGTAGTACATTAGCATAGCATTGTATTTTGATTTTACAGTAGCA | |
| ATTTCATAAAAATGTATCGGTATAGAATTCTGAGTTTGGAACTTTCTCCAGGACACGGTGTTTTTTAGTACACTGTGTCTAAACATTGG | |
| GTATAAACAAAGCATAAGGAATGTGTTTAATGAGTAGCATTACTGCAAAAAAAAAAAAAAATGTAGTCCTACACCAACATGTGGTTCTT | |
| CGTATCCTCCAGTTTGCCATTGTCTTCAAAACTCCAAAGTATAAACATATTAATATTACAAAACCAGCCTCTGTCTTTGTCCAGCTTCC | |
| GAGGAAATCTGACTTGGAAACTAGTGAACCAAAACCTTTCCTCTACTATCCTGAAATCAAAGGTAAGTCAGTTGTTTAAAATCTTATGC | |
| TCATATTTTATTTTATTTTATTTTTGGTTTTTGTTTTGTTTTGTTTTCTTTTGAGACAGAGTCTGACTCTGTTGCCCAGGCTGGAGTGC | |
| AATGGTGCAATCATAGCTCACTGCAACTTCGAACTCCTGGCCTCAAACAGTTCTCCTGCCCCAGCATCTCAAATAGCTGGGACTACAGG | |
| TCTCCGCCACCATGCCGGGCTAATTTTTTTCTTTTTTTACTTTTAATAGAAATGAGGACTTCCTACATTGTCCAGGCTGGCCTTAAACT | |
| CCTCCCCTTAAGCACTCCTCCGGCCTTGGCCTCCCAAAGTGCTGGGATTACAGGCTTGAGCTGCCATCTCTTCCCATTGTGCTTGTTCT | |
| GAGAAGAGGACAAGCTAATTAGAAAAGATCAGTTAGTCACCTCCTTTGAACAGCTTTCTAGTAACAGGTCCCTGGATCCATGGTGCTTA | |
| TTTTTAGAAGAGACAGTAGTATATTATTTTGAGGTCATGGAATTAGTCTAGCTTTTTAAAATAATGTTATTTCCACCAAGCTTATATGA | |
| GATTTAGACATTCAGAATTTGTCTTTGAATTTGAGAATCTAACATTTATTGACTAACTCAGAGTTCAACCAGCTGAGATGAAAAAATCA | |
| GGCTTGCTGTTTAGGAGAATCAGCCAGTTAGTCAACCAGCCTCAGGCGGTGGAACAGAAAACTTGGCCAACATGCACTGTGAAACTATG | |
| AAAATGAGTAAGGCATCATTCTTTCTCTCACAAAACATGCTTTAACAAAGCGCACTCATAGCTTTCTTAAATACTTATAATGTAAATTA | |
| TGATTTTAATAAGTACCGAAAGTGAAGCATCCATAAAAATATAAGAGAATGTTGAAGAQAGAGCCCTTGGTCCCTTTATTCTAAAGCAG | |
| CCTCCAAAAAGAAGGCTTCGTCAAGCAGATATCATTTGGCCCTCAGCATTTGGGGTATATTTCAGCAGTTGGGTGTTAAACAGAGAAGG | |
| AGAGGGTTCTCTAGGTTTAAGTTACTTCAGCAGCTTAAACAGACAGCTAAACACACCGTTTATGCACAGGGAAAAAAGTCTTCTGACCG | |
| CTCGGATGGACATGAAGGCCAATGTGGGTAAGGAAGGCCAGGGCCAGGCTAAAGAGGGCTGTGAATGGCAGCTTAAGAATTTTGCATTA | |
| ATTCTGGAGAGGGATAGCCGGGCCTGCCTAGGTTTGGAATGATGCCTCTGACTTCTTTATGTCACCACTGAGGGTACCATTTACGAGCC | |
| AGATGATGAGTCAGAAGAGTAATGCAAGAATCCAAGTGAGAAACAGTGCAGTTACTAAAGGTCCTAGTGCCAGGAATGAAACGAGATGG | |
| TGGACTTAAGAGAGAGTGCAGTGATACTGTGATACAGTGATACTGTCAAGAGGGCCTAGAGGTCGGAGGTGTAGGAGAGACCGGGAGTC | |
| ATAGATGGGTCTAAGACTCCCTGACCAGGAACCTTGTGATCTGGTTGACTTGATGAAAGCAACAAGGGAGCAAAACTAGATTTCGGTGG | |
| CAAAAGAAGCACCTCAGAGTAAAACATGTTATATTTTAGGTCCCTCTCGGAAAATCAACTGTAAGTTCTTACTAGGCAGATCAAAACGC | |
| AGAACTGGAACTCAAGAGAGGACAGGGTGAGAAATTTAGCATTAGAATCATCTCAAAACGAGATATAACAGGCACCAACTGAGTCATCA | |
| TTCAAAGGTATTTCTTAACCTAGAGTCCACTGACCCCCAAAGAGTCTGTGGATAGAAATGTACTTTGATATCATCAGCTTCTTTTATAA | |
| TCCTGTTTTGTATTTTACATACTGAAAACCATGATTCTGACAACACATCCATAGACTTCACCAGACTGCAGAAGGGGCTGTGGCACAAA | |
| CAAAATTAGAAACCCCTCAGTTGGAGGGAAGTGAGACTGGGAAGGGAGGTCAGTAAAATGATTGCCACGGGGCACCCAGATCCTTGCTG | |
| ACGTTGGAAACCGCAAACGTGCAGCAGCAACAAGCCACATGGGTGCGTGTTCTCTCCTGTGAGAGCAAGATCTGATAGGTAATCTTTGA | |
| AGAGGGCTTTCCTTTCCTAGAATTCGTGCTGCCACAGGAGTGTTTAGAAAACGGTTCTACAAGTTCTTGTTTCTTTTCTGAGGTTGTGG | |
| CCTCTCCATTGTTTGGTCAGATTTGTAGTCCACTCTATATCCTCCTCCTTTCAAAACATTGTCACCACAATAACTTTTCTTTCTTCACA | |
| TGAGATTTCATACAAAATCCCAGTGTATGCACATGCTAGGCTTTGATTAAAATAGCGGTGAAGGGCCCTCCCAGCTCGGCCTTCCGAGA | |
| CCCTGTCCCACCTCTCCTTACTTCCCTATCACAAGAGCTCCTaGAGGTCTTTTGAATGCTTCTCCCTACACAAAATCATGTGAATCACT | |
| GCTTTAATTTAAACACCCTTCTTTAAAACTAGACACAGAAGAAAAATATGCATAATTTTTTTAGACTTTCTGAGGAAAAAAATAAATTT | |
| CTTCTCCAGAAATATGTCTTCAAATAATCCCACTTTTTGCTAACACAGACAATCATGAAGAAACAAAACCCAGGTTACCTGTATGTGTA | |
| TGAACCTAGAAACATTCAAAGCTCACCTTGGTATCTAGGCTAACCTGTCTCCCTTTCCCAGTGGTATCTATCCCTCAGAAAGTATGTTA | |
| TAGTGGGGGTATATTCAGGTGATTACTTTAATGCCTCGTTATCATAGTACGAACTATCCAATGCAGGGATTAGAAATAAGGSTCACCTG | |
| AAACTTCAATATTGCACCTCTATGCTTCATGCACCTACCTTCCAGCCTCTCATTGATGAAAAGCTTCATACAATACAAAGTTTAACAAC | |
| TGTCTGGAGAAATACTTACCCATTGAGCTCTSAAGSAAAGAATGGACAACATATATATATATGAGGACAACAAATTACTTCATTCTATA | |
| GAAACCTTCTCGATTATAAAAACTCAAGATAATASGACCAAGCAGAAATAATCTGACCAGGCAATATAACTGGTCAGAAAAGTAGTAGG | |
| CCATTTAGCATTAGACTAACTACATAGAAATCCATCCAAACTGTACAACCCACGCAAACATTGCATACATTTCTCAGAAGTCATTGAAC | |
| ACACTTTTCCTTACCCCCAGTCTCTGT | |
| HUMAN SEQUENCE - mRNA | |

(SEQ ID NO: 29)
GGCCACCGCACCGGCCCGGCCACGATCGCTGACAGCTTCCCCTGCCCTTCCCGTCGGTCGACCCCCCAGCCGCCGCAGCCCTCGGCCTG
CACGCAGCCACCGGCCCCGCTCCCGGAGCCCACCGCCGCCGACGCCCCACCCGCCCGGCCAGTAACGCGGCGCCGCCCGCGGCCACCGC
GGGCCCTGCCGTTCCCTCCGCCGCGCTGCGCCATGGCGCGGCGCTGACTGGCCTGGCCCGGCCCCGCCGCGCTCCCGCTCGCCCCGACC
CCCACTCGGGCCCGCCCGGCCTCCGGCCTGCCCCCGCCTCTTCCTTCTCCACCCGSCAGGCCCCGCCGCTTAGGACGCAGAGCCCACCC
CCGCCAGGAGGCCGAACGCGGACTCGCCACCCGGCTTCASAATGGCAGAAGATGATCCATATTTGGGAAGGCCTGAACAAATGTTTCAT
TTGGATCCTTCTTTGACTCATACAATATTTAATCCAGAAGTATTTCAACCACACATGGCACTGCCAACAGATGGCCCATACCTTCAAAT
ATTAGAGCAACCTAAACAGAGAGGATTTCGTTTCCGTTATGTATGTGAAGGCCCATCCCATGGTGGACTACCTCGTGCCTCTAGTGAAA
AGAACAAGAAGTCTTACCCTCACGTCAAAATCTCCAACTATGTGGCACCAGCAAAGGTTATTGTTCAGTTGGTCACAAATGGAAAAAAT
ATCCACCTGCATGCCCACAGCCTGGTGGGAAAACACTGTCACCATGGGATCTGCACTCTAACTGCTGGACCCAAGGACATGGTGGTCGG
CTTCGCAAACCTGGGTATACTTCATGTGACAAAGAAAAAAGTATTTGAAACACTGGAAGCACGAATGACAGAGGCGTGTATAAGGGGCT
ATAATCCTGGACTCTTGGTGCACCCTGACCTTGCCTATTTGCAAGCAGAAGGTGGAGGGGACCGGCAGCTGGGAGATCGGGAAAAAGAG
CTAATCCGCCAAGCAGCTCTGCAGCAGACCAAGGAGATGGACCTCAGCGTGGTGCGGCTCATGTTTACAGCTTTTCTTCCGGATAGCAC
TGGCAGCTTCACAAGGCCCCTGGAACCCGTCGTATCAGACGCCATCTATGACAGTAAAGCCCCCAATGCATCCAACTTGAAAATTGTAA
GAATGGACAGSACAGCTCGATGTGTGACTGGAGGGGAGGAAATTTATCTTCTTTGTGACAAAGTTCAGAAAGATGACATCCAGATTCGA
TTTTATCAAGAGGAAGAAAATGGTGGAGTCTGGGAAGGATTTGGAGATTTTTCCCCCACAGATGTTCATAGACAATTTGCCATTGTCTT
CAAAACTCCAAAGTATAAAGATATTAATATTACAAAACCAGCCTCTGTGTTTGTCCAGCTTCGGAGGAAATCTGACTTGGAAACTAGTG
AACCAAAACCTTTCCTCTACTATCCTGAAATCAAAGATAAAGAAGAAGTGCAGAGGAAACGTCAGAAGCTCATGCCCAATTTTTCGGAT
AGTTTCGGCGGTGGTAGTGGTGCCGGAGCTGGAGGCCGAGGCATGTTTGGTAGTGCCGGTGGAGGAGGGGGCACTGGAAGTACAGGTCC
AGGGTATAGCTTCCCACACTATGGATTTCCTACTTATGGTGGGATTACTTTCCATCCTGGAACTACTAAATCTAATCCTGGGATGAAGC
ATGGAACCATGGACACTGAATCTAAAAAGGACCCTGAAGGTTGTGACAAAAGTGATGACAAAAACACTGTAAACCTCTTTGGGAAAGTT
ATTGAAACCACAGAGCAAGATCAGGAGCCCAGCGACGCCACCGTTGGGAATGGTGAGGTCACTCTAACGTATGCAACAGGAACAAAAGA
AGAGAGTGCTGGAGTTCAGGATAACCTCTTTCTAGAGAAGGCTATGCAGCTTGCAAAGAGGCATGCCAATGCCCTTTTCGACTACGCGG
TGACAGGAGACGTGAAGATGCTGCTGGCCGTCCAGCGCCATCTCACTGCTGTGCAGGATGAGAATGGGGACAGTGTCTTACACTTAGCA
ATCATCCACCTTCATTCTCAACTTGTGAGGGATCTACTAGAAGTCACATCTGGTTTGATTTCTGATGACATTATCAACATGAGAAATGA
TCTGTACCAGACGCCCTTGCACTTGGCAGTGATCACTAAGCASGAAGATGTGGTGGAGGATTTGCTGAGGGCTGGGGCCGACCTGAGCC
TTCTGGACCCCTTGGGTAACTCTGTTTTGCACCTAGCTGCCAAAGAAGGACATGATAAAGTTCTCAGTATCTTACTCAAGCACAAAAAG
GCAGCACTACTTCTTGACCACCCCAACGGGGACGGTCTGAATGCCATTCATCTAGCCATGATGAGCAATAGCCTGCCATGTTTGCTGCT
GCTGGTGGCCGCTGGGGCTGACGTCAATGCTCAGGAGCAGAAGTCCGGGCGCACAGCACTGCACCTGGCTGTGGAGCACGACAACATCT
CATTGGCAGGCTGCCTGCTCCTGGAGGGTGATGCCCATGTGGACAGTACTACCTACGATGGAACCACACCCCTGCATATAGCAGCTGGG
AGASGGTCCACCAGGCTGGCAGCTCTTCTCAAAGCAGCAGGAGCAGATCCCCTGGTGGAGAACTTTGAGCCTCTCTATGACCTGCATGA
CTCTTGGGAAAATGCAGGAGAGGATGAAGGAGTTGTGCCTGGAACCACGCCTCTAGATATGGCCACCAGCTGGCAGGTATTTGACATAT
TAAATGGGAAACCATATGAGCCAGAGTTTACATCTGATGATTTACTAGCACAAGGAGACATGAAACAGCTGGCTGAAGATGTGAAGCTG
CAGCTGTATAAGTTACTAGAAATTCCTGATCCAGACAAAAACTGGGCTACTCTGGCGCAGAAATTAGGTCTGGGGATACTTAATAATGC
CTTCCGGCTGAGTCCTGCTCCTTCCAAAACACTTATGGACAACTATGAGGTCTCTGGGGGTACAGTCAGAGAGCTGGTGGAGGCCCTGA
GACAAATGGGCTACACCGAAGCAATTGAAGTGATCCAGGCAGCCTCCAGCCCAGTGAAGACCACCTCTCAGGCCCACTCGCTGCCTCTC
TCGCCTGCCTCCACAAGGCAGCAAATAGACGAGCTCCGAGACAGTGACAGTGTCTGCGACACGGGCGTGGAGACATCCTTCCGCAAACT
CAGCTTTACCGAGTCTCTGACCAGTGGTGCCTCACTGCTAACTCTCAACAAAATGCCCCATGATTATGGGCAGGAAGGACCTCTAGAAG
GCAAAATTTAGCCTGCTGACAATTTCCCACACCGTGTAAACCAAAGCCCTAAAATTCCACTGCGTTGTCCACAAGACAGAAGCTGAAGT
GCATCCAAAGGTGCTCAGAGAGCCGGCCCGCCTGAATCATTCTCGATTTAACTCGAGACCTTTTCAACTTGGCTTCCTTTCTTGGTTCA
TAAATGAATTTTAGTTTGGTTCACTTACAGATAGTATCTAGCAATCACAACACTGGCTGAGCGGATGCATCTGGGGATGAGGTTGCTTA
CTAAGCTTTGCCAGCTGCTGCTGGATCACAGCTGCTTTCTGTTGTCATTGCTGTTGTCCCTCTGC
HUMAN SEQUENCE - CODING
(SEQ ID NO: 30)

ATGGCAGAAGATGATCCATATTTGGGAAGGCCTGAACAAATGTTTCATTTGGATCCTTCTTTGACTCATACAATATTTAATCCAGAAGT
ATTTCAACCACACATGGCACTGCCAACAGATGGCCCATACCTTCAAATATTAGAGCAACCTAAACAGAGAGCATTTCGTTTCCGTTATG
TATGTGAAGGCCCATCCCATGGTGGACTACCTGGTGCCTCTAGTGAAAAGAACAAGAAGTCTTACCCTCAGGTCAAAATCTGCAACTAT
GTGGGACCAGCAAAGGTTATTGTTCAGTTGGTCACAAATGGAAAAAATATCCACCTGCATGCCCACAGCCTGGTGGGAAAACACTGTGA
GGATGGGATCTGCACTGTAACTGCTGGACCCAAGGACATGGTGGTCGGCTTCGCAAACCTGGGTATACTTCATGTGACAAAAAAAAAAG
TATTTGAAACACTGGAAGCACGAATGACAGAGGCGTGTATAAGGGGCTATAATCCTGCACTCTTGGTGCACCCTGACCTTGCCTATTTG
CAAGCAGAAGGTGGAGGGGACCGCCAGCTGGGAGATCGGGAAAAAGAGCTAATCCGCCAAGCAGCTCTGCAGCAGACCAAGGAGATGGA
CCTCAGCGTGSTGCGGCTCATGTTTACACCTTTTCTTCCGGATAGCACTGGCAGCTTCACAAGGCGCCTGGAACCCGTGGTATCAGACG
CCATCTATGACAGTAAAGCCCCCAATGCATCCAACTTGAAAATTGTAAGAATGGACAGGACAGCTGGATGTGTGACTGGAGGGGAGGAA
ATTTATCTTCTTTGTGACAAAGTTCAGAAAGATGACATCCAGATTCGATTTTATGAAGAGGAAGAAAATGGTGGAGTCTGGGAAGGATT
TGGAGATTTTTCCCCCACAGATGTTCATAGACAATTTGCCATTGTCTTCAAAACTCCAAAGTATAAACATATTAATATTACAAAACCAG
CCTCTGTGTTTGTCCAGCTTCGGAGGAAATCTGACTTGGAAACTAGTGAACCAAAACCTTTCCTCTACTATCCTGAAATCAAAGATAAA
GAAGAASTGCAGAGGAAACGTCAGAAGCTCATGCCCAATTTTTCGGATAGTTTCGGCGGTGGTAGTGGTGCCGGAGCTGGAGGCGGAGG
CATGTTTGGTAGTGGCGGTGGAGGAGGGGGCACTGGAAGTACAGGTCCAGGGTATAGCTTCCCACACTATGGATTTCCTACTTATGGTG
GGATTACTTTCCATCCTGGAACTACTAAATCTAATGCTGGGATGAAGCATGGAACCATGGACACTGAATCTAAAAAGGACCCTGAAGGT
TGTGACAAAAGTGATGACAAAAACACTGTAAACCTCTTTGGGAAAGTTATTGAAACCACAGAGCAAGATCAGGAGCCCAGCGAGGCCAC
CGTTGGCAATGCTCAGGTCACTCTAACGTATGCAACACCAACAAAAGAACACAGTGCTGCACTTCACCATAACCTCTTTCTAGAGAAGC
CTATGCACCTTCCAAAGAGGCATGCCAATGCCCTTTTCCACTACCCCGTGACAGGAGACGTGAAGATGCTGCTGGCCCTCCAGCCCCAT
CTCACTGCTCTCCAGGATGAGAATGGGGACAGTGTCTTACACTTACCAATCATCCACCTTCATTCTCAACTTGTCACCGATCTACTAGA
ACTCACATCTGGTTTCATTTCTGATCACATTATCAACATCAGAAATGATCTCTACCAGACCCCCTTGCACTTCCCAGTGATCACTAACC
ACGAAGATCTGCTGGAGGATTTGCTGAGGGCTGGGGCCGACCTGACCCTTCTGGACCGCTTGGGTAACTCTCTTTTGCACCTAGCTCCC
AAAGAACGACATGATAAAGTTCTCACTATCTTACTCAAGCACAAAAACCCACCACTACTTCTTGACCACCCCAACGGGGACGGTCTGAA
TGCCATTCATCTAGCCATCATGAGCAATACCCTCCCATGTTTGCTGCTCCTCCTCCCCCCTCCCCCTGACGTCAATCCTCAGGACCACA
AGTCCGGGCGCACAGCACTGCACCTGGCTGTGGAGCACGACAACATCTCATTGGCAGGCTGCCTGCTCCTGGAGGGTGATCCCCATGTG
GACACTACTACCTACCATCCAACCACACCCCTGCATATAGCAGCTCGCAGAGGGTCCACCAGGCTGGCACCACTTCTCAAAGCAGCAGG
AGCAGATCCCCTGGTGGAGAACTTTGAGCCTCTCTATGACCTCGATGACTCTTGGGAAAATGCAGGAGAGGATGAACGAGTTGTGCCTC
GAACCACGCCTCTAGATATGGCCACCAGCTGGCAGGTATTTGACATATTAAATGGGAAACCATATGAGCCAGAGTTTACATCTGATGAT
TTACTACCACAAGGAGACATGAAACAGCTGGCTCAACATGTGAACCTGCACCTCTATAAGTTACTACAAATTCCTGATCCAGACAAAAA
CTGGGCTACTCTGGCGCAGAAATTAGGTCTGGGGATACTTAATAATGCCTTCCGGCTGAGTCCTGCTCCTTCCAAAACACTTATGGACA
ACTATGAGGTCTCTGGCCGTACAGTCAGAGAGCTGGTGGAGGCCCTGAGACAAATGCGCTACACCGAACCAATTGAAGTGATCCACCCA
GCCTCCAGCCCAGTCAAGACCACCTCTCACCCCCACTCGCTGCCTCTCTCGCCTGCCTCCACAAGGCAGCAAATAGACGACCTCCCAGA
CAGTGACAGTGTCTGCGACACGGGCGTGGAGACATCCTTCCGCAAACTCACCTTTACCGAGTCTCTCACCAGTGGTGCCTCACTGCTAA
CTCTCAACAAAATGCCCCATCATTATGGCCAGGAAGGACCTCTAGAACCCAAAATTTAG

TABLE 6
(human PVT1)
Human transcript (SEQ ID NO: 31)
Human genomic (SEQ ID NO: 32)
Human protein (SEQ ID NO: 33)

| | |
|---|---|
| Human PVT1 Genomic | (SEQ ID NO: 32) |

GAGTTGTCAGCAACCTAGAGCCCTGAATGACTGCCTGGAAGAGCTGCCTCACCACCTGAGCATCTGTGTTAGACCAGCGGGACATTCGC
TAAAGCAATTAAAACATATTTTATTCAATACTACTCACAGTCAGGGAAAGACCTGAGCTCCATTCTAATTTGTGCCCAGGTATCTGGGC
ATTTTAAAGGGAGAAGGAGGGATGGGGGATGGCAGGGCTTGAAATGAGTTAGGGAAGTGAAAAATTACAAAGAGCAGGAAGGGAAAAGG
GCTTGGCCCATGTGACTGAGACTAGCACAGGACCCTATCTTCAGGTGTTGGCTGGAACAAACAGTAAGTTCTTTGAATTTTGTAGGCAG
GCACTTTCAGGGTGGGCTCAAATCGTGCTGGGGAGGCATCCTCGTGCTGTTAGAACCATGTTAGTGTGTGTCCAAGGCTTTTTTTTTTT
CTTTTTTTCAGAGACAGGGTCTCACTCTGTTGCCCAGGCTGGAGTGCAGTGGTGCAATCACAGCTCACTACACCCTTGAATTCTTAAGC
TTAAGTGATCCTCCCACCTCAACCTCCTGAGTACCTGACACCACAAGTGCACACCACCATGTCTGACTAGTTTTTTTAAAAATATTTTT
GTGAGATCGGGTCTCCCTCTGTTTCTTGGGCTGGTCTTGAACTGCTGGGCTCAAGTAATCCTACTTCCTCGGCCTTCCAAAGTGCTGGG
ATTACAGGCGTGCACCACCATGCCCAGCCTGTCCAAGTCTTTTTAGGCTGAGGTTAAGACCTGGTAAGAGATTGGTCAAGAAGAGAATC
TTTGCTATCACCCTATTGGGCTTATTCCATTAGTTTACAAATGAAGCCCAGGAAATACTAATGTAGATGGGTCCCAGCAAGAATTTAAG
TACTGGTAATTCTTAACAAATGTTTATGAAGAGTTTATTATGTACCAAACTCCCTACCACACCTACATCCACAATCTTCACACCTACAT
CCACAATCTTCACACCTACATCCACAATACCACATCTACATCCACAATCTTCATTTGCTCCTCTATTTGCAAGATCTTGTCTTTACAAA
AAAAATTTTAAAAATCATTAGCCAGGTATGGTGGTGTGTTCCTGTGGTCCCAGCTACTCAGGAGGTTGAGGTGGGAGCATCGCTTGAGC
CCAGGAGTTTGAGGCTGCAGTGAGCTAGGATTGCACTATGCACTCCAGTCCGGGTGACAGACTGAGACCCTGTCTCAAAATATAATAAT
AAATTAAACAAACAAACAAACAAACAAACAAACAAATAAGTAATCTGGCCGACGCAGATACTGAACGTTCGTTAACACAATCCCAATTC
CTAATCCCTTTCCTCCTTGACTGCTTTTGCTACAGAGGTTGGAAAACTCCAACACTTGCTATCCCAGCTTTACTTGCAGCTAGCAGAGG
CCATATGAGAGCTCTAGCCAATGAGGCTGAATGTTTTACCACGGGTTGGTTGGAAGGTTTTTGCTTTTTCTGGCAAACAGGACATACTT
GGTTTAGGCTGCTTCTTTTACTTATTCCTGCTTTAACCATGGATTTGATGCCCAAGACCAAGAATATGCTGCTCTGACAATACTAAGCT
CAAACAACAAAGAGTATTGTTTTTTACATAAGAAGAGGAAAAATGATCTCTTCTTTTATCTCTCACTTACAATTGTCTAAGTTTTCCCA
GAAGCTCCCCAATAGCTTTCCTTTCACATGCCATTGGCCAGAATTGGATCAGCTGAGCCCATTCCTGAATTGATCACTAAGAAAGGAAG
TAGAATCACATGCTAGGCTTAGACTGATCACTGGGTAGAATGGATACTGGAGCATCAATCAAAGCATGCCCTATAGCAGGTAAATGTGA
TCACAAGACAAATAATGACTTTCTGAATATAAAAACCATAGGAATAAATAACAAATTAAATGACTGGTGAATATGAGCTTATTGACATT
AAAGCAAATTTATAAAACCACTCATAAACAAAAGGAAAAGGCAAATAAGACATCAGGGATATATTTTTGCAATTTACATGGCAAATAAA
GTGTTAATATCTCATAAGTCACTTCTAAAAATTCACCTTAAGGAAATCATTAAGGTCATATGCAATGATTTAGTCACAAAGATGTTTGT
AACAGAACTGTGTTTAATAACGGAAGTTTGCAAGCAAGATGAATTCCTAGCAATAAGGTACTGGTTAGGTAAAATATGATACAAACCTA
CAATGGAATGCAACAGGACTATTGAAAAACAACGTTGCAGAAATATATGTACCGTTATCAAAAGATGATTGTGATGTGTTATTAAGTGG
CAAAATCAGAATACAAATAATGCACATGGTTTGTTTCTATATACCTGAAGAAAAATGTAGGAGTATGTGTGTGTGTATTTGTGTGTAAT
TATGTAGAAGGATGTGTATTCCAGAGTTAACAATGGCCATTTCTGATGGATTTGTTGACTTTTCTTTTTCTTTCTTTCTTTTTTTTTTT
TTTTTTTGAGACAGAGTCTCGCTCTGTCACCCAGGCTGGAGTGCAATGGCACAATCTCGGCTCATTGCAACCTCCGCCTCCCAGGTTCA
AGCGATTCTCCTGCCCTCAGTCTCTCAAGTAGCTAGGATTATAGGCACCCACCACCACGCCCAGCTAATTTTTGTATTTTTAGTAGAGA
TAGGGTTTCACCAAGTTAGTCAGGCTGATCTTGAACTCCTGACCTCAGGCGATCCACCCACCTCAGCCTCCCAAAGTGCTGGGATTACA
AGCACCGCTCCCGGATATATTTGTTGATTTTTTAAACTAGTCAGCATTCAGGTTGCATTTGTAATAAAATAAACAAGTAAAGGAACTTA
ATTTAGGGAACATGTATCAAAAAGTAAATGAGCATCTCAACAGCAAAGTGGTCAATGGGCCTAAGGAAAATATGTTCATAAAATAAAGT
ACAATGAGCCAATGATCAGGTGTGTAAGAATTTATTCTTGGTAATAAATCAGTATGTAATTAAAACAAGAATGTAATGTCTCTCTTTTT
ATTCCCAATCCATTCAACTGGAATGCTTTCCCCGCATGAAAAGAATAGAGCCTGGTTGGGAGTACAGGGAAATGTACAAGTTCACAGGT
AAAACTGTGCTGAAGAACAATTTGTCAATAGGTAGACATTCAAAAATGCCAATTTTCTTTTGTCTGAGTTATTATTATTATTATTATTA
TTTTTATTTTTGAGATGGAGTCTCACTCTGCTGCCCAGGCTAGAATGCAGGGGTGCCGTCTCGGCTCACTGCAACCTCTGCCTCCCAGG
TTCATGCAGTTCTCCTGCCTCACCTCCTGAGTAGCTGGGATTACAGGCACAGGCCACCACACCAAGCTAATTTTTTTTTCTTTTTTTTT
TTTTTTTTAGTAGAGATGGGATTTCACCATGTTGTCCAGGCTGGTCTCAAACTTCTGACCTCAGGTGATCAGACTGCCTCGGCCTCCCA
AAGCGCTGGGATTATAGGTGTGAGCCACTGCACCTGGCCCTGGTCTAGGCAATTATTTTTAAATTATTTTTATTTTTATTTTTTGAGAC
AGGATCTCACTCTGTCACCCAGGCTGTAGTGCAGTAGTGTGATCATAGCTCACTGCAGCCTTGACCTCCTTGGGCTCAGGTGATCCTCC
TACTCAGACTCCCCAGTAGCTGGGACTACAGGCTTGCACCAGCATCCCTGGCTAATTTTTGTACTTTTTGTAGAGATCGGGTTTTGCCA
TGTTGCCCAGTCTGGTTTCAAAATCCTGGCTCAGGTGATCTTCCCGCCTCACCTTCTCAAAGTCCTGGGATTACAACCATGACCCATCA
TGCCTGGTCTGATTTTTTTTTTTTTTTTTTTTTTTTTTTCAGACTGAGTCTCTATCTGTTGCCCAGGCTAAAGTCCACTCGTGCGATAT
CAGCTCACTGCAACCTTCACCTCCCGGGTTCAAGCGATTCTCCTGCCTCAGCCTCATGAGTAGCTGGGATTACAGGCTTCCACCACCAA
GCCCAGATGGTTTTTTCTGTTTTTAGTGGAGACGCGGTTGCACCATGTTAGCCAGCCTGGTCTTACACTCCTAACCTCAGGAGATCCAC
CTGCCTCAATCTTACGAAGTGTTGGGATGGTGGGAGAATAAAAATGATCAATGTGGTTCCAGGAAGCGCTGATTCATTAGGAGCTGGCT
TTCTTTGTCTCTCTTGCTTGAAATAAAACTAGTCTTGGCTTCCTGTCACCTCAGGGGCAGCTCTGCTGTAGAAGACACAGGTGACTTGT
TCCTGGTTCTGCCACTTGCTGATTTCCTTAGACAAGACTTCTTTGGGCCTCGGCTTGCCTGTCTGACTTCACAAGTCAGAATTATAACA
ATGTCCCTGCCCGGGAGCTGTTCAGAGGCTGAAGAGGAACACCAGACTCAGTAAAGCCTCCAGCCTGCCAGGTAGCGCTGGGCCCACCA
TGACCTGAGTCCTACCACGACAAGAAGAGGCACCCTAGAGTCTTCTGTGAAGTGCACATAGAAGAGAGACTGGGCCCAAGCCACAAAAG
ATAGAATGCACAGCTCGGCCCTAAACAACCATTTTCTAAGAGAAAACAAAAAGCCAATGACACAGCACACCTACATACTGTGTGGTTAG
AGTGAGACAACAAAAATCTGCATCTTCATAAGATAAATCCGGAGAGACTATTGAGAGTCTATTAGTGGCAGAGAATTGAATTCAGAGGG
GCCAGTTCTCCTGCTGCACAATCCAGAAAAAAAAAGACAGAAGCCGACAAGGCACAGTGGCTCACACCTGTAATACCAGAACTTTGGGA
CGCCCAGGTGGGAGGATTGCTTGAGCCCAGGAGTTTGAGGCTGCAGTGAGCCACAGTTGCACCACTGCACTCCAGCCTAGGGTGACACA
GTGAGACTCTGTCCCGAAGAGAGAGAGAGAGAGATTGATTGAAGAGATGGAAGCTTATTCCACAGCAATGACCGTGAGCATGAAGAAAG
GTGCGTCCCTCAATCTATAGGACTAATCAGTAGACAGACATTGGGAAGAGGAGAACTCAGGTTCATGGCCTCAGCAAGTGGTGCTACCT
GTCATAACCACTCTTGAAGCCTTGAAGCCTGTTATGTCAGGCAGAAAGTAGGGGACAAACTTTCAAATCATACATCTACAAAGAACATT
CGTCATGCACTCCAGCCATCCTCAATTGCTAGCAGTACATCAACCTATGCACATACACACTCTTCCCTTTCCTAAAATGCTCCCCATTC
TCTTGGACAAATGGCAAACTCCAACCTATTTTTTGAGTTTCAGCTCTGGCCCCACCTCCCTGGGGCAGTCTTTTCAGCCTTTGCCAAAT
ACATACCTCTCTGTGCTTCCACCACACCTGGGGTTACCCCCTCTCATTCTGTTCTCACAACATCTAGGATCTCTATCCCTTCGTTCACT
CATTGGTTCTATATGGAGCTACTGATATGTGATTCCTGACCCAATAGAAAATGATGAGGGTGGCTGTTTGTCTGACTCAAGCCTTTGTG
AAATGACTGATGGTTCTCTAGGCCTTTTTCTGCTGTTCTCTCTGCCTGGAACACTCTAACACTCTATCTGGTTTTTATGGCTGGCTCCT
TCTTTTGTTTTTTTTTTTTTTTTTTTCCTTTCACATCGACCCTCTACCTGTCACCCGGTGGAGTGCAGTGGTATGATCTCACCTTACTC
CAACCTCCACCTTCCAGGCTCAAGTGATTCTCCAACCTCTGCCTCCCAAGTAGCTGGGACTACAAGGTGCGTTGCCACCACACTCGCCT
AATTTTTGTATTTTTTGTAGACATGGGGTTTGGCTACGTTGGCCAGGCTGGTCTCGAACTCCTGGCCTCAACTGATCTCCCTGCCTTGG
CCTTCCAAAGTGCTGGGATTATAGGCATGAGCCACCACACCTGGACGGCTGGCTTCTTCTTGTTGGTCAAATTATCTAGTATCAATTAC
CTTCTTGCCCCATTTTCTCTTTAGCTCTCTGCCGAGATTTACTGTCTCAATAGCATTGACCATTGTTGAAATTACCTTGCTTATTGGCT
TGTCTCCTTCTTCTGACTAGAATCTCAACCTCACAAAAGCAGGGACTTGGGCTGTCTTATTGCACTCATAGTCCTGAGGACTACCACAG
TGCCTGGCACATAAGAGGAATTTGATAATGTTTGATAAAGGAATGAATGGGTTTTGTAGATAAGGATGCAGATATAGGGCCAGGCATGG
TGGTTCATGCCTGTAATCCCAGCACTTTGGGAGGCTGAGATGGGCAGATTACTTGAGGTCACGAGTTCAAGACCAGCCTGGTCAACATA
GTGAAACCCCTGTCTACTGAAAATACAAAAAGTAGCTGGGTGTGGTGGCAGGTGCCTGTAATCCCCACTCAGGAGGCTGAGGCAAGAGA
ATCGCTTGAAGCCAGGAGGCGGAGATTGCAGTGAGCCCAGATCGTGCCTGGGTGACAGAGCTAGACTCCATCTCAAAAAAAAAAAAAAA
AAAAAAAAAAAGAGCATGCAGATATAGAGAAGGTAAACACTGGTACTGAGCCTAAAATTATTTAATTATAGAACCAATACTAAACAACC
ATGAGAACTGCATTTACAGAATGGAAGTGGTAATCGAAAGACAATAGCACAACTAGGCTAGCGCAGTGGCTCATGCCTGTAATCTCAAC
ACTTTGGGAGGCCGAGGCGGGTCAATCACTTGAGCCCGGGAGTAAGCCAGGCATGGTGGCACATACCTACTCAGGAGGCTGAGGTGGGA
CCACTGCTTGAGCCTGAGAGCTGGAGGCTGCTGTCAGTCGAGTTTGCACCACTGCTCTTCAGTCTGGATGACAGAGTGAGATCCTGTCT
CAAAACAAAAACAAAAACAAAAACAGAAAACAACGAACAAATACAGGTGAGGTAAAGGGAGATGTGAGAAAATGTGTGAATGTAATAGC
AAAAAGTTGCCTCGACTTTACAATGATGAGGCCTACAAGTAAAACATCATCATGCCACTCTTTCCTTGCTTTTCATTATCTTTTCTGAA
CCTTTGTAGCCTTATTTAGAAAGCAATGCTTCTCATGTGGTAAATAAATATTTAAAACACAACAATTCCTTTAGTTTTATGTTAGCTTC
CTTTTCTTCTACTAAAAAAATTAGATTTCTTAAAAATTGCAACTATGAGACATTAACATTGTAAAATATTTTTCCTATCTATTTGTCAA
TCCTGCATCCATCTATATATCTATATCAAGATCTATGCCTATATGTATCCATCTGTGCCCAAAAAGCCCTCATCTGATATTCACAAAAA
TTATTTCTGGCTGATAAAATTTGTTTTTCTTCTCTATTTTTTTTTCTGCAGAGTTTTAATGTTTTACAACCTGTATGTATCTGTGTGTG
TGTATATATATATATAATATCACTTTTGCAAGAAGAAAAGTGACTTTTAAAAAATGAAAGCAGGCCTGGGCACGGTGGCTCACGCCTGT
AATCCCAGCACTTTGCCAAGCTCAACCGGGTGGATCACGAGGTTACGAGATCGAGACCATCCTGGCCAACATGGTGAAACCCCGTCTCT
ATTAAAATACAAAAAATTAGCAGGGCGTGGTGGCGCACGCCTGTAGTCCCAGCTACATGGGAGGCTGAGGCAGGGGAATCGCTTAAACC
CAGGAGGTGGACGTTGCAGTGAGCCGAGATTGAGCTGCTGCATTCAAGCCCGGTAACACAGCAAGACTCCATTTAAAAACAAACAAACA
AACAAACAAACAAACAAAACAAAACAAAAGCAGATATACCAAAATGTTTATTTTGGATGGTAGGAATATTAAAATATTATTTTTTCTTT
GTATTTTTACTGAAAAAATCTTTTAATTCTAAAAAAGAAATTAATAAAAAGGAAAATGTGAATCTAAGATTAGTGCATGAGAGCTGGAG
ACCCAGACAGCGCAAACACCATCAAGCTCAAAGTTGAGTCAGTCCTGGGTGAAAGTGTCTTTTCAGCCACGGGACATTTTCCATGCAGA
AGGGACTGGGCATCAACTGTCAGGACATGGACAGAGACGTCTGCCATTAGAAGCTGGTCCTCTCATAGCCCACCAGCATCCCCCAGGAG
GACACTGGCTTCCTCAAGGTCACAGCCTTTGGTTTCTGGAAAAAATGATTAAGCAGTGAAAGGATTAAGGTCTCACAGCTCCTATCGCA
TGATGTATAGATGCAGAGGTTTCTCATCATCTTCTTTTTATATTTTAAAAGATGGATGACAACTAGATGCCAACTGCAGCTCAGGCACC
TGCCCAGTCCCTCTCAGAGCCGGGCACAAAACAGACACCTGTCTAGCCCCTCACAGAGAGCCAGGCACAAAACAGACACCTGTCCAGCC
CCTCACAGAGAGCCAGGCACAAAGCGGAGACTTGTCCAGCTCCTGGCAAGGAGCCAGGCACAAAAGAGATGCCCATGAGTGTAAATGAA
TCTGTGAAACAGGCAGAACAGGAGCATCAACTGAGTAATTGTTTTTTCTTTTTCAAATAAGAACATTTGCGTTGAGAAGCCGCCACGGG
AAGAGATGTGATGAAACACTACACAGAAAGAGGGAGGAGCCTCCGGGTAGGTTCGAGTCAGTAGGATGTAGGGTACCCGGCAGGGCAGA
AGAGGGGCAACCTTCTGTTCCATAATATTGATACCACCCATTTGTGTGTCCAATTTTTGCTGGTTTGTGACGTCCTTCTAGGGTCCATG
AGTAACCCCCACCCCATTCCCTAGCTGGGTGCCTGACTCCCCTTAAGTGCTCAGTAGACGTTGAACTGGACGGGCGCTGGCGTGGTGGC
TCACGCCTGTAATCCCAGCAATTTGGGAGGCCGAAGCGAGTGGATCATCTCAGCTTCGGAGTTCGAGACCAGCCTGGCCAACATGCTGA
AACCTTGTCTCTGCTAAAATTACAAAAATTAGCTGGGCGTGGTGGCGCGCGCCTGTGATCCCAGCTACTCCGGAGGCTGACGCAGGAGA
ATCGCTTGAACCAGGCAGGCGGAGGTTGCAGTGAGCCATGATCGGGCCACTGCACTCCAGCCTGGGTGACAGAGTGAAACTTCATCTCT
AAATAAATAAATAAATAAATAAATAAAATAAACTTTGAACTGAACAGGGCCACCTCTTACATCTGTCCAGTCTCGGGTTTATGCAGATA
GAACAAGATAACCACATCCCACATGGGAGGCCAGTGTCCACTTGCTCCTTTGGAGGAACTACTGGGCATTTAAGGGACTCGTTGAACAC
ACTCTGTAGGTCAACTCTGCCCGGAGTGGAGGGAGCTGCGCAGACCGCTGGCCCTCTGCACGGGTTTGCAACTCGGCTTCTGGAAACAG
CGGCAGACGTCGAACGAGGCGAGAAGGTGCGCGCGCCCTTCTCACCAGGCCGGCGGATGCTGGCTGGTACCCAAAGAGGCGCTCAGATC
ACTGGAGCTGGAGCTCAGTCGGCGGTCTTCTGCGGGAACTGCACCTATCCCGGAGCATCTCTGGCCCTCCTATTTCACTGAACTCGCCT
TCCTCAGCAGGAAAGTGGGAAGACCGCTTGCTTCCAGGACGGTGTGGGGAGGGTAAGAGGGCTCAGGGAAAGACCGTGGAAAGCTCGGC
GCACAAGCGAGCTGGCATCACCGGGGATTCCCTCCGGCCCGGTGGGACTCCGGCGCGCTTGTAGCGAGGACCCCCAGCCCAGGGCAGAG
CCTACCCTCCGCTCCCCCAGTCTGGGCCCTTGGCCGCCCCAGTCGCCCAAGCCCGCCCCGCCTCCGCGCGCGGCCCTCTCCGGCTCAGT
GCCCTGCGCTGCCGGGAAGCAGGCTGAGGGGCGCACCGGGCGGCGGGCGGGGACCTGGGGAAGGCCGGGAGCGCCGGGACCGAGGACGC
ACGCGGCGGGCCCCGCCGCGCCCGCCTCCCCCCTCCCCCGAGGCCCGAGCGCGACCCGCCGTGACGTCACGGGCAACCCGCCAGCCCCG
CGCTCCTCCGGGCAGAGCGCGTGTGGCGGCCGACCACATGGGCCCGCGGGCCGGGCGGGCTCGGGGCGGCCGGGACGAGGAGGGGCGAC
GACGACCTGCGAGCAAAGATCTGCCCCCGGACCCCCGCCACCTTCCACTCGATTTCCTTCCCCAAACGATGTTGGCGGTCCCTCTGACC
TGTGGAGACACGGCCAGATCTGCCCTCCAGTAAGTTCCAATTTTGTCCCCTGCGTTTCTGGAAACTCTCGAGACTTGGCCCTGAGTAGG
GATGCGCTGTGAGTAGTCGGACGGAGGAAAGGGGCTTTCGGAATCCAATATGTGTGTGCTAGGCGTGCACTGTCCTCATCACCAGCCTC
AAGACTGGTGGCGAGTTTGGGCGTTTGGGGAGGCGGAGGGGAGGCGCATGACACCGAATGGCCGGTCCATAGACCCAATTCTGGATTTA
TGAACTTCATCAAACGCATATTAACTAATTTCTAAAAATACACCCCTCTGCAGTCAGAACAGATTTGGAGCCATCATGTGCTTTGGACG
CCCCTCTGGCCAAACTAGAGGTCTGTGAATGTGTGATCCTCCCTCCATCCTTTGACCATCACTCCCCGCTTCCTCTCACACCCCCTCAG
GGCTGGGCGAAGGTGATTTCCAAAAACTCAGTAACTCCGTGTTTTGAGACTTTCTCAGTTTTTGCATCGCATGTGAGGGTTTGTTGGGT
GCTCCTAAACCCTCATCATGGTCATTCTTCTTGGCTGGGCTGTTGATTTAATTAGGTTTGCCTTCTCTTGCTAACGTGCTCTTATTTAT
GCCAGTGTTTCTGGTTCTGCGTCTAGACTTTCACGAAGCACACCCATGAAGCTGCATGCGTCTGTGTCTGGATCCCTCATCCCTTTTCC
TGGTGGTTTGCAGGCATGCAAGATTCTTCTGTTCATCTAACCTCCTCACTTTAGGCCCTTCTAGAACATTAGAGACGACAAGAAATCAC
GTGCAAGGATTTAGGGACAACAGGATGAGGTTTTGGTGTTTCCTTTCTGGTATTAGGTTTTCCAGACACACGCTGTTAGCCTGTCTCTC
GCCCACTCCACCACTGTGAATCCTCGCTACTGGGGCTAGTTCCTTGCTAGGAAAAAAAAACTCTAGACAAATGTGTTTGTGTGTGCTGG
GTGGGCGGGGGCCCTGAGGATGCAGGGACTGTGTCTGCTGTGTTGTCTGTTATAAGATGTTTCAGACCTATTTTGCATACTGGCAGCGA
CAAGTTGAGACTTGTTCAACTTGACACAGTCCTCTGGTCATAGCGAATCTTTCTAAACCTCTGATCAGTCAAGAAGGGGGTTGTATCAA
TCCTCAGAACCCTGAGTGGAACTTTCTACAGGATTTATTAGGAGAAAAACCTTCCCGGAAGCTGCAGAAGGACAAATACAGAATCCGTG
TCTCGCAGAAACCTCGTGGCCTGGTCTCCATTATTTGAGATGAGTTACATCTTGGAGGTGAGGACGTGCCTCGTGGTCTAAAGCTTCGG
CACAAGGGCCCAACTGGAATTCCACTTACGGGTATCACTGTGCGGTATATGCTGTACCCATTGAATTCCCACAGGCATTTGTTAAGTGC
TTGTGTTGCTGTTTTGCCTGGTTAAGAGTGGCTTATTCTTGTTGCCTCATCTATCTTGAGTGCAGAATTTTTGCATAAACGGCTTCTCT
TGAAAAATACAAGTCCGTTTTTGTTTTGTTTTGTTTTTTTTCTGATTAACTTTCCTTTTCCTCTGTCTTAAACACCTTCCCCCGCCCCT
CGCCACCTCCATGCTGTGTTTCTGTGGCTGCAGCTTTTCTGCACTGGAAAGGAGGAGTTCTCCCCTCTGTTCTGACATATCTGATCCCC
ACTAGTTTATATGATTGCTGGTCTGAAAACCTGGTGAATTCTCACTGCCCACATCAAATCAATTCCCTGTTCTTTAGTTGTTTTTTTTT
TCCGCCTCCTGAGCGGTAGGCAGGCACTCTGGCTGGATTCTGGAGCCTGTGTTTGCCATGCCTGGGCCTTCTGCCTGCCCAAGATGGAC
ACGTTTCTCTGGCAGCTTCGTCAAGAGATCTCCTGGACTATAAGACACCCTGTTCTTTCCTCCTGTTTGTGTGAGTGGCCCTGCATTTG
GGCAGAGTATTGGCAGAATATCTCGGGGAACTGGAAGACTTTGCATAGTTCCCGGTCCAGCTCCTCTGGAGAGGCAGTGGCAGGCCTCC
TGATGGCTGCCCCTTTGCTATTTGTTTTATATCAGGTGGCCCTTCTGGATGTCTGGCCTTTTTATTTTGCATTTCCTTTCATAGTTGGC
TGCACACATAAGATTCTTCTTTTTCTCTTGTTTTGCTTTTTTTTTTTTTTTTTTGAGACAGAGTTTCACTCTTGTTGCCCAGGCTGGAG
TAAAATGGCTGGATTTCAGCTCACTGCAACTTCCGCCTCCCAGGTTCAAGCGATTCTCCTGCCTCAGCCTCCCAAGTAGCTAGGATTAC
AGGCATGCATCACCACACCCAGCTAATTTTGTATTTTTAGTAGAGACGGAGTTTCTCCAGATTGGTCAGGCTGGTCTTGAACTCCCAGC
CTCAGGTGATCCGCCCCCCTTGGCCTCCCAAAATGCTGGGAGGCCTCCCAAGTTGCTGGGATTACAGGCATCCACCACCATGCCTGGCT
AATTTTGTATTTTTAGTAGAGACGGAGTTTCTCCAGATTGGTCAAGCTGGTCTCGAACATCTGACCTCAGGTGATCTGCCCTCCTCGGC
CTCCCAAAGTACTGCGATTACAGGAGCCACAGTACCCGGCCTTGTTTTGCTTTTTATTGTTTCCTCATGCAGGTTTGTGATCTCTTTGA
GGGATCAGGACCACACCCCCCACCCCAATTTATATTCCCAGTGTCCATGGTGGGACCCTGACTGAGGATGGGAACAGAGCAACAATCTG
CAATCATTTGGACCATTGAGTCCTGTGGACAGTCACTTCAATATCATCCCTAGAGAGTCTCAGAAATTCAAGGCCTTTTGGAATGCTGC
CAAAATGGGTCCTTTGGCAGGAGCCCCCTTTGTCTCTTCTGTTTTCTGACCTCAAATAATCTAAGGAACTAATAACGGTAATAAATAAC
AATTAATAATAAGAACAATAATAGTGCATGTATAACATCGACATTGTTTATCACGCAAAGCATGTTCTTCACCTTCTACTCCCTACAAC
TCCATGAGGCATTTATAATGAAATGTCTAGCAATGGTAGACTTCTTGTTGTGGGGCAGGGTTGGTTCTAACACTTCAAATTATATGATC
TCATTTAACTTTCCTATAAAATAGGATGATTATTGTTGTGGTTTTGCAGATGAGGAACCCACAGCCCAGAGAGGTTAAAAATATCCTCC
CAGGTCACACAGCTTGGGAGAGGCAGAGCTGTAGAGATTTGGACCACAGTCTTCGACTTCTTAGTGTGACTCTCAGAAGTTAGAGGATA
ATGACGTTAAGAACAAGCAAAGGGCCGGGCGCGGTGGCTCACACCTGTAATCCCAGCACTTTGGGAGGCTGAGGCAGGTGGATCACCTG
AGGTCAGGAGTTCGAGACCAGCCTGGCCAACATGGTGAAACCCGGTCTCTACTACAAATACAAAAAATTAGCCCGGCATCGTGCCCCAC
ACTTGTAATTCCAGCTACTTGGGAGTCTGAGGCAGGAGAATTGCTTGAACCCGGGAGGCGGAGGTTGCCGGGAGCCGAGATCGCACCAT
TGTATTCCACCCTCGGCAACAAGAGCAAAACTCAGTCTCAAAAAAAAAACAACAAGAAAAGGGTATACCCCTTGGCCTTCAGCAGGAGA
ACACAGTGTGTCCCCCACCCTCACCGTGCACCTTACGCACAAACACAAAGCTGAACATCTTGCATGAAGATACCTCATGCTCTGCCTGC
CGACCTTTTCTGCAGCCACGGAGCAGTCAGAGACAAAGGGGCCAAGAGGCCTTAGGTGAGGGCTGCCATGCTGGAAAGTTTGGGCTGTG
GGTGTGGGCAGGAGTCATGCCTAGAGCTCAGAACCCTTAGTCTTGGCTGGGTGCAGTAGCTCACACCCGTAATCCCAGCACTTTGGGAC
GCTGAGGCCGGTAGATTACCTGAGGTCAGGAGTTCAAGACCAGCCTGGCCAGCATGGTGAAACCCTGTCTCTACTAAAAATCCAAAAAA
AAAAAAAAAAAAAAAAAATTAGCCCGGCGTGGTGGCACACCCCTGTAGTCCTAGCTACTCGGGAGGCTGAGGTAGGAAGATCTCTTGAA
CCCGGGAGACGGAGTTTGCAGTGAGCTGAGATTGCCAGTGCACTCCTGCCTGGGAGATAGAGTAAGACTTTGTCTCAAAAAACAAACAA
CCGCCTCGGTCTTTGTGCAAAATCTATAATCATTTGCAAGAGGCTGCAATCAGGTATAGCACTGGATTTTGTGACCAAAACTAGGTTTC
AGGATTTAATTAAAAACAAAAACAATGTCAGGTCATAAAACACAAGACATATACATGATGTGGAAATGGTCTTATTCATTTTTTAATTG
CGAAATTCCGAACTATCTGTTTTTTTTTTCCTTTATCTTTTCCTTTTTGTGTGTGTGTGGAGTTAGGTGGGGGGGGAGGTATGGCATAT
TGTTTTGTTTATCTGCCTCTTTATTATCACTGACTGTGAAATTCTTGAGAACAGCGGCCTCTAAGTGCCCAACACAGCCCTGGTACTTT
GTTGAATCAGGTGGCTGTCAAGAGCTGCTGCTTCAGCCCGGCATGGTGGCTTATGCCTATAATTTTTGCACTTTGAGAGGCTGAGGCAG
GAGGATTGCTTGAGCTGAGAAGTTTGAGACCAGCCTGGGCAGCAGAGTGAGACTCTGTCTCTGTAAAAAATAATAAAATAAAAATTAAA
AAATAAAGAAAGAACTCCTTTGTCTTCAAGGAAACAAGATACACAGTCAACACTCATTATCTTTTTAGATATCAGACAGCTTGACATGG
TGTGGAAGTGGGGGGCTTGCCTGTAGGGAAAGTATGCTGATGAAAAAGAAGGAAAACAGACAGTAGCAGGAGGCAGTGGGGATGTGTCA
GGGAGCTCAGGAGTGTAGATCTGGTTACTTTTGCGCAAGTTACTTCTCTAAGCTTCAGTTTCCTCATCTATAAAATGGTTTGCTGTGAT
GACGPAAGAGATCATGAACACAATGTGTCTGGCAGAGGACCTGGGCTCATAAGTGAGCACACAGTAAAAGGTAGCTAATATCATTACTG
TTCCAATTATTGAGCTTTATTACTTCCTGTGGCTGCATGAAGTAGTGGTTTCTGAATAGAAAGGCAACATTCTAGTTGCCTAGATCTTA
AGATGCCAGTGACTTCACTGTAGTTCTCTGCAAGCATCAGAAACAAGCCCTGGCTTATGTAAGTTAGAAACAGATGAGAAAGTAAATAA
AGCTGCAGAAGTGGGTCTCACGAAGCACAGCAATCAAAGGGCTGTCCCTCGGATGTCAGACTGCCTTTCCAACTTACTTCAGGTCACTG
TGCCTCCATTTCCTCACCTGCTGAGCAGGGGAGGAGAATCACTTCTCCCTCCCTGGGATGGTGCAGAGCACTTAGCAGAGCACTTAGCA
CAGAGGAATAAGCCCTTCAGTAAAACATTTGAGTAAAGCAATTATTATTTATTATTCTTCAGGGCTCTTTTTTTTTCTGGTGCATTAGC
CCTAACATTCACTACTTCGTTCAAGAGTGATGGATGCATTCATTTCCTGAGCAGTTGCTGTATGCCTGGCACCGTTTTAGCTGCTGGCG
ATCCAGCAGGGAGCAGAAGAGACAAAAATCCCTGCCCTCCTGAAGCTTCTGTCCTAGTGGATGTCACACAGTAGTGGGGCAGGCTGGTC
CCCAGAGACAGCTGGGGGACAGATGGAGGCACTGCAGGCAAAAGCTAACTCCTGTGCAAAGTCTCCCCGCCCCCACTGTGTTAACAGTA
AAGTGCTTAGCAGAGGCTCCCACACATAGTAGTTACTATGTAGTGCTAGTTGCTATGTTTATTATTATTATATTATTCTCCTCCCTCAT
AAAATGCCAGCCTCCCTTTCTTCCATTCCCCTTCCTGAGTTATTTTTTCTCGATAGGGCTTATCTTCCTAGTATACCATATGTCTCTCT
TCTCCCACTAAATTGTAACCCATTCATTTTTCCAGGATATATTTGTTGGGTGTCTACTCTGTGCCTGGCATTGCTGTAGATGTTGGGGA
TGCAGAAAGGAAGAAAAAGGACAGATTTCCATGCTAGAGCTTAGGTTACAGCTGCCTTAGACACATAACGAACAGCACACATAATAAAG
AAGGACATGAATTGTGTATTCAACATTATCAAGTGCTGTGCCAACAGGCCTAGCAGGGTCACTCTCTCGAGGGCTGGGGCGTGGTGCTG
TGTCGTTAAGTTGTCCCCTCACAGTCAGGCCAGCCTCCATGGAGGAGCTGACCTTAACCTCCACCTCAAGTACGCGAACGACAGGCGTT
TCAGGCTCTCGGAAGAGCTAGGACAGACGTGGACGCAGAAGTCAGCCTGTGGGGGTGAGCAGCACCCAGGAGAAGCGTGAGATCAGAAT
GGCGTGAGCAAGGGACGTACGAGAACCCGAGCAGCATCCACTCTGCACCCTCACAGAGAGCCTGCACGCCATTCCAAACATTCTGTCTT
TTACTCTGTAAAATGGCCACCCCTTGCCGTCTTTTTTGTTTTTGTTTTTTGTTTGTTATGAGGCAGAGTCTTGCTCTGTTGCCCACGCT
GGAGTGCAGTGCGGAGATCTTGGTTCACTGCAACCTCTGCCTCCTGGGTTTAAGCAATCCTTACACCTCAGCCTCCTGAGTAGCTGGGA
TTACAGGTCCCTCCCACCACGCCTCCCTAATCTTTTTATTTTTCTAGAGATGGGGTTTCACCATGTTGGCCAGGCAGGTCTCAAACTCC
TGAACTCAAGTGATCCACCCACCTCAGCCTCCCAAAGTGTTGGGATTACAGGTGTGAACCACTGTGACTGGCTTCCTTGCAGTGTTCTA
AAAAGGAAAGTAATTTGATCTGACTTGGCTTTTGAAACGGTCAGTCTGGCTGCTCTCTTGACAAGAAACTAAATCACGGTCAGGAGCTA
GAGCACCAGGACAAACATGCACACAGCGTTAACTGCCACGTCCACCCTCGCAGTGGTGGATGGGGCCACAGGAATGGACTTCTAGGCTG
TTCACTGTTGTATCCCCAGCATCTTAAACTGTGCTCTCCATATGGTAGCTGTGAGCCCTATGTCGGTATTGAGCACTTGAAAGGAAATT
GATATCTACTCTGTGTTAAACGCACATTGGATTTCAGAGACTTAGTCAGAAGAGTAAGATATCTCATTATTTATTTATTTATTTATTTA
TTTATTTATTTACTGAGACACAGTCTCACCTGTTCCCCAGCCTGGAGTGCAGTGGCATGATCTCGGCTCATTGCAACTTCCGCCTCCCG
GGTTCAAGCGATTCTCCTGCCTCAGCCTCCCAAGTAGCTGGGATTACAGGTCCCCACCACCACACCTGGCTAATTTTGTATTTTTAGTA
GATATGGGGTTTCACTATGTTGGCCAGCCTGGTCTTGAACTTCTGACCTCAGCTGATCCATCCCACTCAGCTTTCCAAAGTGCTGGAAT
TACAGGTGTGAGCCACTGCACCAGGCCTATTTGTTTATTTATATGAGACACGGTCTCACTCTGTCACCCAGGCTGCAATGCAGTGATGT
GTTCATACATCATTATAGCCTCGACCTCCCACGCTCAGCTGATCCTCCCACCTCAGCCTCCTGGGCAGCTGCGACCACAGGTCTGTCTC
ACCACGCCCAGCTAATTTTTTTGTATTTTTGGTAAAGCTGGGGTCTCCCTGTGTTGCCCAGGCTGGTCTTGAGCTCCTGGGCTCCAGCA
ATCTGCCTTCCTCAGCCTCCTATAGTTCTAGGGTTACAGGCGTGAGCCACTCCACCCTGCCAAGTTATCTCATGAACCAATTCTTTGAT
CTTAATTACATATTCAACTGATAATGTTTTGGATATATTGGGTTAAATTATTAAAATTAATTTCATCTGTTTTTTCTAACTTTATGGCT
ACTAGAAAATCTAAAATTACATGTGGGTTGAATCATATTTTCGCTGGATGGAACAATGCCTTGCACGAGGTCAGCATTTAGTGAATCAA
ATGTCTGGGTGAGGCACAGGTCTTCTCCCGTCAATCACAAACCGTTCCCATCTTCGGGAAGCTTCCAGTGTGGGAGGGAGGCACACTGC
AGATGTAACTGCTAGGAGAGAAAGAAGGCAGGTTGAAATGGGACCACAGCCAACCAGGCCTGCTGTAGCCCAGAAGCTGCAACAGACTC
GCCCAGGAGGGAAGGAAGACACTCGGGTGTTGGGTCTCAGTCTGGGTCTTGGTGACAGCAGTGGCCTGGCAGTGCACAGGTTCAAGCCC
TTGGACTGCAAGTCCACTAAGGTGGCTGGTGGTGTGGGTGGAGGTGGAAAAGGCAGGTGGGGCGAGGGTGAGGGGCAGGACATCCTGGA
GAGCCTGAATAACCAGGTATAAGTGATTTCTTTTTCTTTCTTTCTTTCTTTTTTTTTTTTTTTTTTTTTTTTAAGACGGACTCTCCTTC
TGTCGCCCACGCTCGAGTCCAGTGGCGAGATCTCCGCTCACTGCAAGCTCCGCCTCCCGGGTTCACGCCATTCTCCTGCCTCAGCCTCC
CGAGTAGCTGGGACTACAGGCACCCGCCCACCACGCCCGGCTAAGTTTTTGTATTTTTAGTAGAGACGAGGTTTCACCATGTTAGCCAG
GATGGTTTCGATCTCCTGACCTCGTGATCCGCCCGCCTTGGCCTCCCAAAGTGCTGGGATTACAGGCGTGACCCGCCGCGCCCAGCCCC
AGGCGGAAATTATTTCAACTGGGTCCTGAGAACTGGGCACTTTTCAAGGATTGTCAGCAAGGAGCAACTTCCCAAATATACATCTAAAA
AGATGACAAACTCATGTGGAATGTGGATTTAAGGAGGAAGGTACTTGACTTAAGAATTCCACGATTTTGGTGGAAGAAGCAAATATGGC
CCAGGCACTGTGGCTTATGCCTGTAATCCCATCACTTTGGGAGATAGAGGTGCCAGGATTAGTTGAGCCCATCTGAGACCAGCCTGGGC
AACATAGTGAGATCCTGTCTCTACAGTTTTTTTTTTTTTTTGAGACGGAATCTTGCTCTGTCATCAAGGCTGGAGTGCAGTGGTGAGAT
CTCAGCTCACTGCAACCTCCGCCTCCTGGGTTCAAGCGATTCTCCTGCCTCAGCCTTCCGAGTAGCTGGAGTTACAGCCTTCTGCCACC
ATCCTTCGCTAATTTTTGTATTTTTACCAGAGACCGCCTTTCACCATGTTGGTCAGGCTGATCTCGAACTCCTGACCTTGTGATCTGCC
TGCCTCGGCCTCCCAAAGTGCTGGGATTACAGGCATGAGCCACTGCACCTGGCCTCAAAAATTCTTATTTTTAATTAACTGGGCTTGGT
GGCATGTACCTGTAGTTCCATCTTTGCTGGCACGCACCTGTATTCCCATCTACTTGGGAGGCTGACGCACCAAGCTTGCTCGAACCCAG
AACTTTCAGCCTCCAGTCAGTTATGATTACACCACTGTACTCCAGTCTGGGTGACAGAATGAGACCCTGTCTCTAAAATAAATAAATAA
ATTTTTAAAAGCTGGGAGGTGGGGTTGGTAAATTCATAAAAAATAATATTTCTTGAGTTGAATACAAACAATACAATCACTGTTTTTTC
TTTGCCTTGTTTTATTTTTACTTATTTATTTATTTTTGCAGACATTGGGCCTCACTCCTACCCTGGTCTTTAACTCCCGGCTTCAAGTG
ACCCTCCCACTTTGGCCTCCCAAAGTGTTGAGATTACAGGTGTGAGCCACTGTGCCCAGGCTGTGGTTTTTTATTTTTTAATTTTTTAT
TTTTTTGTATTATAATCATAGTGTATATGTTGCATCCTGATTTTTTTTTTTTTTTTTTTTGACACAGCATCTCATTCTGTTATCCAGGC
TGCAGTGCAGTGGCACAATCTCAGCTCACTCCAACCTCTACCTCCCACGTTCAAGCGATTCTTCTGCCTTGGCCTCCGAGTAGCTGGGA
TGCCACCGTGCCCGGCTAAATTTTTTTTCTTTCTTTCTTTTTCTTTTTTTTTTTGTATTTTTAGTTCAGACAGGGTTTCACCATGTTGG
CCAGGCTGGTCTTGAACTCCTGACCTCAAGTGATCCCACCTCGGCTTCCCAAAGTGCTCGCATTACAGCCTGAGCCACCACATCTGGAC
ACATCCTGATTTTTTTCTTAGTTGCTATTAGGACACACGCATTTTTGAATATTATATGCACTTTGTCCATGTCTTTTTTTTTTTTTTTT
TTTTGAGACGGAGTTTCACTCTTGTTGCCCAGGCTGGAGGGCAATGGCGCGATCTCGGCTCACTGCAACCTCTGCCTCCCGGGTTCAAG
TGATTCTCCTGCCTCAGCCTCCTGGGTAGCTCGCATTACAGCCGCTCACCACCACGCCCGGCTAATTTTGTGTTTTTAGTAGAGACGGG
GTTTCTCCACGTTGGTCAGGCTGGTCTCGAACTCCTGACCTCAGGTGATCCACCCGCCTTGGCCTCCCAAAGTGCTGGGATTACAGGAG
TGAGCCACTGTGCCCAGCCGTCCATGTCATTTTTATTGGGTAGATATTTCTGTTCAGTGACTGTTTCCTTGCTTACTTTATCATTCCTT
TGATTTGGATGCTTAGCTTCATAACAGCCTGTTTATATAGATTATGTTTTCTATTTTTCAGTTAATTAAAAAAAAAACAAATTCTCAGT
CCTAGGATTATTGTGCATAATGTCTGAATTTTTTAGAATTTCAGGTAAATATTGTGGTTCATGAGATTTGCACAGAAGCTGAATTTGCT
GCTTTCTCGTGAGGCACGTGATGTTTTTTGCTGTCCGTGATGCACTAACATGTCATTAACATGTATGTACAGTGTTATATGCTCGCACA
TGTTTATGTGCAGCCTATGCACCTGTATTGGATTTTTCTTCACTCTCATTTATTGGCAGGTATTTGTGGAATGCCTGTCCTCTGCCAAG
TTCTCCTTGAGGCCTTGGGTATTCAGTAGTGATCAAAGTAGATCAACTAGTACCAGTTCTTAAGAGGCTTATTGTTAAGTGCGGCAAAC
AAGCAAATAGACAAAGAAAAAATAACATTATTGTAAGGAATATGAAATAATATGTACTAGAATAATATGAAGGGATGGTTATTGTGAGG
TAAGGGGGTTCATACTGAAATACTGTCTGAGGAATCAGGATTTGAGTGAGACACGAAGACTCAGTAGAAGTTAACCAGAAGAACAAACT
GTTAGCAGAAAAGACAAGATTGATCAATATGACTACATATTAAAGAAAAAGGCACATCTAAGCAACAAAAACTGCAATGGTATTAAAAG
CCAAATACAAGACTTAGAAGATATTTGCAATATTTATAACACATTACAAAACACTAATATCTAGAATGTATGCAGAATTCCCAAAAGTC
AACAACAAAAAGACAAACAGCCTTATAGCAAAGTGGGTGAAAGATAGGACCATGCAGTGCTCATACAAAGCAAAGTAGATGGCTGGGCA
CCGTGGCTCATGCCTATAATCCCACCACTTTCGAAGGCTGAGGCGGGCGCATCACAAGGTCAGGCGTTCGAGACCATCCTGGCCAACAT
GGTAAAACCTCATTTCTACTAAAAATACAAAAATTAGCTGGGTGTGGTGGCGTGTGCCTGTAGTCCCAGCTACTTGGGAGGCTGAGGCA
GGAGAATCGCTTGAACCTGGGAGGCATAGGTTGCAGTGAACCCAGATCTTGCCACTGCATGCCAGCCTGGACACAGAGAACGACTCCGT
CTAAAAAATTAAAAAAAAAAGCAAAGTGGATGATAAATGTGAAAAGATTCTGAATTTTGCTAACAAAGTCAGCTTTTGGAGATCTAATT
TCTAGTCAGTAAAATTCATCCTTTCTACCATACAATGCTAACACTTTTGATAAATGCATAGAACTGTGTTACACTGTTGTGTAATTACC
ACAATCATGGCTTAGAACATTTCCATCGTTCACCCAGATTCCCTCCTGTCCCTTCACAGCCAGTCTCCTTCTCCACCCCCAGCTTTAAC
CAACCCCTGAGTTGTTTGCTGACCTTACAGTGTTGCCTTTTTCAGAGTGTCCTATAAATGCATTCATACAGTGTGTAGTCTCTCGGATT
TGTCTTCTTTCACCTGGCATAATGCATTCGGGACCCTTCCATGTTGTTCTATGCATCAGGGGTTTGGTAGTTTTTATTGCTGACTGGGA
CTCTCTTCTATGGATATACCACTGTTTGTTTATCTGTTCTCCATTTGAAGAGCATCTGGGTCTTTCCACAAGATACATGTTTCATGCTT
TACAACGTGCCAGAAATAGGATGTCTGACTCAAGTGTTGACAAGGCACAGGGTGTTCAGCAGCTCCGCTAGTGAGTGTGTGAGGTCACT
TTAGAGACCACCTTGCCAGCAGGTAGTGAAATTGGAAACATGTGCAGCCCCCTTCCTAGGTAACATTGAGAATTCACAGTTCGTCCTGT
ATGTTACTTAAGCATATTTAGAGATGAAACTGAAGGTTTAAAAAATGGGCTGGAAAGATCTATATCAAACTCACTGTCATCATCTGGCG
ACACAACGGCAGTGGCCTCATTGTGAACCTCAAAGCCTACTTTAACTTTGCCTTTATTCCTTTTTTTAAATTCAATTAATTAATTTACT
TGTGGCGACAGAATCTCACTCTGTCAACGTTGCTGGAGTGTGAGTGGTGTGATCGTGCCTCACTGCAGCCTCTACTTTCCAGGCTCAAG
TCATTCTCCTGCCTCACCCTCCCGAGTAGCTGCGACTACACGTGTCAGCCACCATGCCTGTATTTTTTCTAGAGATGAGATCTCACTAT
ATTCCCCAGGCTGGTCTTGAACTCCTGCCCTCAAGCCATCCTCCTGCCCAGTTCTCCCAAACTGCTCGGATTACAGGGAGGACCCAGTG
TCTGGCCTATCTTTAATGCTTTTTTTGAAAAGAACCTCCATACCTACACATATCACTTACACACTTTCAAAATATGTAGAAAAGAAAAA
AAGTTTCAAAATACATATGACAACNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNCGTGGTGGCTCATGCCTGTAATCCCAGCACTTTGGCAGGCTGAGGCGGGCAGATCACTTGAGG
TCAGGAGTTCAGCACCAGCCTCGCCAACATGGTGAAACCCCATCTCTACTAAAAATACAAAAATTAGCCAGGAGTGCTGGCAGGTGCCT
GTAATCCGAGCTACTTGGGAAGCTGAGGCAGGAAAATAGCTTGAACCCGGGAGGCGGAGGTTGCAGTGAGCTGAGATTGAGCCATTGTA
TTCCAGCCTGGGCAACAAAGTGAGATTCCATCTCAAAAAAAAAAAAAAAAAAAAAAAAAACAAAGAAAGAAAAGAAAAAGAAAAGGACT
TCTTGGAGGGCAAAGTTTGAGGGAAGAGTTAGCTAGCTAGAGACCTTCCAAACTGTGCTCAGGCATTTCGATTTTACCCTGTGGGTGAT
AGGGAACTATTGAAAGTTTTCATTTTTTTCTTTTTGGAGACAGAGTCTTGCTCTGTTGCTTCAGGCAGGAGTGCAGTGAGCCATAGTGG
CTCATTGCAGCCTGGACCCCCTGTGCTCAAGCAATCCTCCCACCTCAGCCTCCAGAGTAGCTGGGACTACAGGTCCATGGCACCACCCC
CAGCTAATGTTTTGTTTTTAATTTTTTATAAAGATAGGGTCTCACTTTGTTGTCCAGGCTTAAAACTCCTGGCTTTAAGCGATCCTCCC
ATCTTGACCTCCCAAAGTGTTGGAATTACAGGAATGAGCCATCATGCTCGGCTCTATGGAAGGTTTTGAGGTGGCGCAGGGATGACATT
ACAGTCATGTCCCTTTCCCAGAGAGATTTGTCCAGAGGAAGGGTGACCAGCCTGGAGTCTCTTTGGAATGTTCTAGGTAATGTAGCAGA
GATTGTGTCAGTTACCCTCTGGTGGGATGCTTGGCTTTTCCCATTTTTTCAAAAGGAAATGGGAAAAGGAAAAGGTCCTCTGTATGGAA
AGTCCCCAGGAATTTATAGGAGAGTGGCTAACGAAGACAGAGTTCCCTTCCGCCATTTATTCTGACTGACTTTCTTCCATACTTTTAGT
CCCACTTAGGTCATGATTCATCCCACTGGGCCAGCAGCAGCATTGGGATCACGTCAGCCAATCTTCGCACTTAGAGATTTTGAAGAGAG
ATATCTCCTATCCATGTGGTACATTGCTTTTCACAAAACAAAAGAGTTCATTTTCTCGGTGAAAAGCCCTGGCCAGACCCAGCCGGTGG
AGACCAGTAGCTTCCTCTGGCAGGATGGGCTTCCTATGGTCATTGCAGTCCCTCCCCATTTATAAGCCTGCCTTCCCTGACAACACATG
GGACAGATGCCCCCCAGAAGCATGCCCTCTGGAAAGGGAGAGGAGCAGGAGGGGGCTTCAGGCATGCTGGGCCTGGGGGGCCACCCCCC
TGGCCTTTGATAAGTTCCTGGCTGGTATGAGCCATCTTCTTTCCTCTCCCCAGGCCTTGCAAAGGCCATATCTCTTCCTTTTCATGACT
CTGGAAATTCCATACATACTTTCTGCATTAGGAAGACATTCTCTGCATGCTGATCTCGTTAAAAAGGAAAAGCCAAGGAGCATCCTGCA
CCTCCCTCCTGTAAGCGAGCTTTGCAAATGGTAAACTAGATCCTGAAAGGCAACCACTTAAAAGCTTTTAATGGCTTCCCATTATTAGC
TTGAGTGCCTGGTGCGTCAGACGCTCTCAGTGACCGTGTATTGAATTGCTGAATGCATCGTCGCCCTGGGCGAGGAATCTGGCCCTCAC
TGTCCCAACCCTGAGTGCCTTTGCCCTGTGGAGGAAAGCACTGCCTTGGGGTCAGAAATAAGGATTGGCAGGATTGCCTGGGGTTAGAA
ATAGTGATCATGGGATCAGAGGCAGCAGGTGAATGTGGGGTGTGAATATTTTTGAAGGCTTGGAATGAAATGATTCTTCCAGACTTTAC
AGGATTTGTGTCTGCAGATTCTTCTCTAACGTTTCTGTCTCACATAGTGAGAGAATCAGCCAGCCAGGGAAATGGGACAGGATTTTGTC
TCTTTCCCCATTTCCTCTTCTAATGAACTCCTACCTATGCTTTATGACTGCATTAAGGGGTCACCTCCTCCAGGAAGCATTCCCTCATC
TCCTTACGCTGGTTCTGAGTGTGTCGTTCCTGCTTGATCCCCTCCCAGCACCATGTGTGTGAGTTCTCACAGCTCACACCCCATAGTAT
TACTATGTTGTTTCTACAGTGGGTTCCTGTCAGGGGTTCCAAACTTAGCTGTTTAGGAGGATGAGTAGATTGTACAACCACACCAGGCG
GGCCTGATCAAATACAGTAAGAAGTGTCCGGATTGGCAAACCAGCGGGTCCCTCTTCTAGATGTTATCAGAGAGATACTGTTAGAGAGA
GGTTGCTAGATGTTAATAGAGGCTGCTGTTACATTCCAGCTCCCTCCTCCCCTCGTCCCTTTTCTCTCCTCATCCCAAAAGATAGACCC
AGTGTTGCCAGAGCTTCCACTTTTAAAAGAGAAGCCTTTTAAAATATAAACTCTCCTGATTTTTAAATGTTTTCAGTTAAGTTTTTAAA
GGAACATTCATAGGCCAAACCAAATACCTGTGGGCCGTATGTGGCCCCCGGGGCCAGCAGTTTGCAGCTCCTTGGTTATAGGTCCCTCT
AACCCATTCCACAAGCTCCCTCAGGCATTTCATGTCTTTCCTTTCCATTCCCCCTGGGCCTAGCAGGGAGCTGGCATGTCACAGTGGGT
GCTCCCTAATTATTGGTTATTGACTTGAATGCTAGACTGAGCAAGCACCTCCCTGCTCACAGGGTGTTAACATTTTTCCCTCAGAACTG
TTTGTATGTAATGCTATTCTGAATTGCTTCAGTTCCTTACATACTTTAGTGATAGTCTGTATCTGCCTGGAAAAGTCATCATTCCTTAG
ATTCTAGACAACTCTGTTCTTCTGTTCTTTTCCTTTTTCCTTTCTCTCTCCGTCCCCTCAACATATGTTTTTGTTTTTTGTTTTTTGCC
AGTTCTTTTGTATCTGGCCGGTTAACACACGGCTTCATAACATCGGGTCCGCTGTTATTAACCAACAGTGGTTTCCGCTAGCTCACTGA
CTTTGCCCTCTCTGCTGGTAACCCATTCCTGATTTCCATCAGCGTAACAAGCATCATTTTCAGATTTACCTTCATTTGCAGTGAACAAT
TATAGCAAAATTGCTTAAAGAAATGAACCTTCAGGTGTGGCGGCTCGTGCCTGTAATCCCAGCACTTTGGGAGGCCGAGGCGGGAGGAT
CACTTCACCCCACGAGGTCTCTAGGCTGCAGTAAGCTCTAGTTGCACCACTGCACTCCAGCCTGCGTGACAGGGTGAGACATCGTCTCA
AAAGAAAAGAAAGGAAAAGAAAAGAAAAGAAAAGGAGGGGAGGAGAGGGGAGGGGAGGGGAAGAGGGAGAGAGGCAGGGAGGGAGGGAC
GGAGGAAGGAGGAAAGGCAGGAAGGCAGGAAGGCAGGAAGGAAGGAAGGCAGGAAGGCAGGAAGGCAGGAAGGCAGGAAGGAAGGAAGG
AAGGAAGAGAATGAAAGGCTGGGCGTGGTAGCTCTTGCCTGTTGTAGTCCTGGCACTTTGGGAGGCTGAGATGGGAGGAATTGGAAACC
AGCCTGCGCAACATAATGAGACTTCATCTCTACAAAACAAATTTTTTTTTAATTAGCCACTCCCATGCACCTGTGGTCCCAGGAGGCCA
AACCTGGAGGATTGCTTGAGGATGGGAAGTTGAGGCTGCAGTGACCTATGTTCCCACCACTGTACTCCAGCCAGAGTCACAGAACAAGA
CCTTGTCAAAAAAAGAAAAGAAAGAAAAGGAGAGAGCGACGGAACGAAGGAACAAAAAAAAGAAGGAAACGGACACAGGAAGGAAGGAA
GGAAGGGAAGGGAAGGGAAGGAACGCAGCGAAGCGACGAAGGTAGGAAAATGCAACGAACGGAGGGAAGGAGGGAAGAAAAGAAAGAAG
GAAGGAAAAAACCGGTCGGCTATCTTGACGAAAAAAGTCTCAGTCCACTCCATTTTGTGCAGCATTTCCCGTCTTGTTATCTTTCTGGA
AAAACTCAGTAAGTCTCTGCCTGGGAATTATAGAAGCTTAATATGCACTTGTAACTGTGTCAGGCTCTCACAGGGAACAAACAAGAGGA
ACTATAGATTTTTATACTCTCCAAGAGCCCACTCCATGCATTTACGAGGCACAGAGTCAGAGAAAGAGCATTGACAATGATAATATAGC
ATTAATATAGGTACAATCTGGAGTAATGTTATCTAGTACTACTAATACCGATATTAAAATGCCATTATATACATATTAAGCCCTCACTT
CGTGCCGGTTTGAACTTTTCCTGTATTAATTCATGTGAATCAATTTTCACACTCTCTCTCAAAAAAAATATATCTATATAATATATACA
AATATATATAATATATACAAATATATATAATATACAAATATATATATATAATATATACAAATATATAATATACAAATATATATAATATA
TACAAATATATATATATAATATATAGATGTCATCCTCATCGGAAGACTCCGGCAGGAGACGAGCAGTTTAATCCCAGGATCCCATCCAC
AGCAAACTTCTAGGTCTCTTACCCAGTGTCTTCTCCTGCTCTCTCGCTCTCATTTCTGAACTAACAAAAGTCTGCAAACATCATCTGCT
CTCCTGGTTGTATTTGTAAATGCCTCCCCTAGACCCAGCTCTACTTTAGGCCACGTCAGTGACAGTGACCACATCACTCAGGCATTCTC
CTACTGTCCCTGACCCATCTCCGCTTTTCTCTCCTCCCACTCTAATAACACACCGTCTCCCTTCAACCTCAGCCCTGCTGCCAAGTACC
TGTCTCAGCTTCAGAGCCTGGTGCCTCAGTTCCCTCATCTGTAAAATGGGAATACAAATGGTCCCTGTCTGTTTGGCTATTCAACAAAA
TTAACTCAGTTCCTGCACACCTCACCTCTCGCTCCTTCAACAAAAACATGGTCACCCTCAAACCAAACGTGAACACCAGCCTCCTCACG
CAAACCACATCCAAACGAGAGGCAGACCCAGCCTCCACTGCAATTGCACGATCTCGGCTCACTGCACCCTCTGCCTTCCAGGTTCAAGC
AATTCTTCTGCCTCAGCCTTCTGAGTAGCTGGGATTACAGGCACCTGCTGCCAAGTCTGGCTAATTTTTCTATTTTTAATAGAGACGGG
GTTTCGCTATCTTGGCCAGGCTGGTCTCGAACTCCTGATCTCACGTGATCCACCCGCCTTGACCTCCCAAAGTGCTGGGATTACAGGCA
TGAACCACCGCCCCCGGCCGCAGATGGGAAATTATTTTTGCACTTTCAGTTGTCCTAAATCTAGACTTTTCCTTTATCCACACAGCTGG
TGAAAGTCGACGTGAGAATAGACAGATTTCCTGGTGGGCAAGACCTCCATCCTCCTTCTCCACCCCCTTCCTCATTATTCCCAAGAAAA
GAGCTTCTGGGTTTCTGGGAGAGGCTGAGCAGGTGGAGACACCTGGGCCATATCTGGGTCTCTCCAGAATCCAGGAGCCTGCCTGACGC
GTCAGCCGGGGAGGGTTTCTGTGTGAAGTCGGCTTGAGTGAGCCAGCCTTTCATGCATCCTTGTCTTTATTTCTTCCCCAGCTCAGGAG
GGGCTGAGTTGTGTCTTAATGAGCAGTTCCAGCTGCATGCTGGGCATCTCCTAATTTACTCTCATTTATTTAGCTCCTGCTGCATGCTG
GGCAATGCTCTAAGTGCTCTGCAGGTTTTGGCTAATTTAATCTCCCTAGCAAACCCTTTGAGGGGACTGACAGGATGACCATCCTCATT
TTACAGATGTCGGGATTGAGGCACAGACAGTTTATTTTTAATTTTTATGTGTAAAATGTTTTTACTGTGGTAAAATTTACATAATAAAA
TTTGCCATTGTAACCATTTCTAACTGTACAATTCACTCCCATTAAGGCCATTGGTATTGTTGTCTAACCATCACCGCTAATCTAACCAT
CTCCGGAACATTTTTCATCATTCCAAACTGAAACTCTGCACTCATTAAACACTTACTCTCCTCTCCCCCTCCCCTCATCCCCTGGTATC
CACAGTTTCTATGAATCTAACTACCCTAAGCATCCGTATAGGTGGAATCATAGTATTCATCCTTTTGTGACTGGCTCATTACACTTGGT
GTAAACTTTTTTTTATTTTTTATTTTTTTTTGAGACAGAATCTTGTTCTGTTGCCCAGGCTGGAGTGCAGTGGCACAATTTCAGCTCAC
CACAACCTCTCCCTCCCAATTCAAGAGATTCTCCTGCCTCAGCCTCCCTAGTAGCTGGGATTATAGTCGTGCACCACCACGCGTGGCTA
ATTTTTGTATTTTTTAGTAGAGACGGGGCTTCGCCATGTTGGCCAGGCTGGTCTCAAACTCCTGACCTCAGGTGATCCACCCTCCTCGG
CCTCCCTAACTGCTGGCATTACACGCATGAGCCACCGTCCCCCACCTTACTGTAATCTTTCAAAGGTATATCTCCGTTGTAGCCTGTGT
CACAATTTTATGCCCTTTTAAGACTGAATGAGTTTTTAAATTCATTCCCATTTAACTCTTTGTTTTGTTGCCTGTGCTTTTGGTGTGAG
GCACAGACATTTTAAGAAACTTAGCCAAGGCCATTCAACCAGAAGTGGCAGAGCCTGGATCTTGACCACATGGCTTTGCTGCTCCCAGA
GTCTTGACCCTGTGCTGGGGCTGACTTCTGCTTTTACATGAGTTCTTGTCAGTGCTTTCGGCAGCTTGATCAGGTAGATGCTGCTTTTC
CACTTTGCAAATATGGAAGGGAGCACCCAGCTCTCTTGGGCATGGCAGAAATTCATCAGGGGCGGAGGAGGCACAGCGTACCCACCCAA
AAAATCCCAACATATTGAATCACCTTCCAATAATCTATACCATCATTTATGTTATGCTGCCCACAACATAAATGGCTAGCAAATAATTT
GGATGCGACAGAATTGGTGAATGAATTTAATCTAGCTTCAACCTCAACACCATTATACACTATTAATACCTACAAAATTATAGATGTAA
GGCTGGGTGTGGTGGCTCATGCCTGTAATTCCAGCACTTTGGGAGGCCAAGGTGGGCAGATAATGAGTTCAGGAGTTCGAGACCAGCCT
CGCCAACATAGTGAAACCCCGTCTCTACTAAAAATACAAAAATTACCTGGGCTTGGTGGCACATGCCTGTAGTCCCAGCTACTCAGCAG
GCTGAGGGAGGAGAATCTCTTGAACCCGGGAGGCGGAGGTTATGGTGAGCTGAGATCACGCCACTGCACTCCAGCCTAGGCAACAGAGT
GAGACTCCGTCTCAACACAAACAAAATTATAGATGTCACCTACAGGCCAGTTGGGAGGCATATTGGTAAGTAAATACCCATGACCTCTC
CACCCTGCCTACGATCCAAACTATGAACACTGATGGTTCACTCTAACTCCATATTCTTTTCACTTTTCTCCCATGCCTACCCCCTGCCC
CCGAGCATTCTCCCTCATTGGTGGCCCTGATTTTCTTTTTGTTGAGATAGGATCTCACTATGTTGCCCAGGCTGGTCCCAAACTCCTGG
GCTCGAGAGATCCTCTTATCTCGGCCTCTCAAAGTGTTGAGATTACAGGCGTGAGCCGCTATGCCAGGCCAACTCTTATCTTGAATGTT
GATGTTTATCATGTCATCCATGAAAAAAAAAAAACCCATGCGTATTCTTTTTTTTTTCTTTTTTTTTTGAGATGGTGTTTCGCTCTTGT
CACCCAGGATGGGGATGGAGTGCAATAGCATGATCTCGGCTAACTGTAACCTCCACCTCCTGGGTTCAAGCGATTCTCCTGCCTCAGTC
TCCTGAGTAGCTGGGATTACGGGCACTCGCCACCATGCCTGGCTAATTTTTGTATTTTTAGTAGAGATGAGGTTTCACCATGTTGGCCA
GGCTGGTCTTGAACACCTGACCTCAGGTGATCCACCCGCCTCGGCCTCCCGTTTGTATTCTTAAATAGTGTATTGTTTCCTTTACTTCC
TTTAGAGCTTTACAGAAATTCCATGCTGCCTTCAGCTTCCTTTGCCGCTCTGCATCATGTTGGTAAGTCTCTGCTGATGCCTGCTGCTG
TAGCTTATTTTTCACTGTGATAATCTGTTCCATTGTGTGAACTGACCACAGTTGATTGATCTGTTCTCCAAGGACATGTGAGTTGTTCG
CAGGTGTCCCCCCACCCCCTCCCGATACAAACAGTGCTGCTGTGGACATTTCATACATCTGGTTGCCTATGTCAGAATTTCCAGGGTAT
ACAGCTAGGACGCAGTACTGCTGTTGGATTTTCCTGGGATTGTAGACTCTCAAATTTCGAATGGACTTCTATGGCCAAGTAGTTCACCT
TCAGCAATCATCTCAAAACAAATGCTCTTGGTGTCATGCTTGACAGCGCTGTTCTGCAATAGGAGAGACACAGACCTGCTGGGCCTTTG
CCTCATTTTATTACTTAGTTCATTATGCAATGAACTTAACATCTGTCTACCTCAGTAGTTTCCCTGTCTGTGAATTGAGTCTGTTACAT
ATATATATATCTATTTAAATATATATATCTATTTAATATATATTAAATAATATATATTTAAATAGATATATATATATTACAGACTCAAT
TTAACCCTTAGGACCCCAGGAGCGGATGAATCGTAACCCAGTTCNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNCTTCCAGGCTTCCCATTTTAGACTTGAAATGTAGGCCTGTTGGGGCCTCCTCCAGTAGATGTGTGCCCTTGAGCTTGGATACCC
CCCTGCCAAGATACGCCTTTCAAAAACAGCATAAACTTATCGCAACTGAATAGAAGGCGCTCCCAAGCGGTCCCACCCTGCTCCGGAAG
TCTCAGTGAGGAATCAATAGCTGTTAATTCCTGTGCTGCCCCCTGCCTGACATTCCATCCACACTTGAGGCCCAGATTGTAAGAAGGGA
GAGCAGGCAGGTCTCTCTTCCAGGAGAGGGACCCGGTTCATCAGCAGGATTTATTCATTCATGTATTACGTCAGGCTCAGCGCCTGGGC
TTTTTATAAATGAGGGCTCTGCTGGCGTTCTGTGTTATTCAGATATTCGGTGAGAGACCACCAGAACCCACAAAAAATTGCTGACATCT
GTTGGAAGGCAATCATGCATAATGGATAAGGGTGCACTCTGCAGTTCCAGCAGCATTGATTTGAATTCCCCCTCTGCCGCTTCAAAACT
CTGACATCTCAGCGAGTTGCTTACCCTCTACAGCCTCCACGTCCTCATCTATAAAACTAGAAAATAAGTGGTACTCACTAGATCCTTAC
CTTGTTGTGAGAGTAGAATGACGTGCATGTAGAGCTTTCCCAGAGCTGCCATGTGGTACGTCCTCAATAAACGCTGGTTATTCTCTCTC
TCTCTCTCACTCTGTTGCCTAGGCTGGAGTGCAGTGGCGTGATCATAGCTCACAACAGCCTCTGTCTGCCAGGCTCAAGGGATACTCCT
GCCTCAGCCTCCTGAGTAGCTGCGACTTGCAGGCAAGTGCTACCCCTCCTAGCTAATTTGTTTTATTCTTAGTACAGATGGGGTCTTGC
TATGTGGTCCCTGCTGGAGTGCTGGTTGCTCTCTAACTGGCTGCGAGCCCCGGGCTAGGCACACTGGTACTGGGGCTATAGCAAAATCC
GTGCTCGGTAAATCATCATTGAAGGAGCAAGGGTGCTTTACAGTTGTAGAATCTCATAGTACACGTTACTTTGGTTTTTCTCTTCCCAG
CTCTGGAGTCCTGGGATCCAAGTGTGAGCCCCATGTCTGCAAAATGAGGCTAGTCACTTGAATCCTCTTGGATCTTGCTTTCCTCATCT
CTTAGTGAGCTTGTAGATAGCTCTGACTGTCACCAGGCTTTGCTCTGAGCACTCTGCATCTGTATTCAACCTGTATACTTAATCTTCAC
AACAGTTGACCTCGTTGATCCATAGTAATTTCTCGTTGTACAAACGGAGAAACTCAGGCAGAGAGATAAGTGTCTTCCCAAGCTTAACA
GCTAACCAGTAGCAGGGCACGCTGGGATTTGAAAGACTCTCTATGTGTCCTAACTGGTATCCTCTCCTGTCTAGAGGTGATCTCTTCCT
CCAGGGTTCCAGGTAGATTGGTTGAGATGTGCAATGCGGAAGAGCCTCGGGATAAGTCGGCTAATGACACCCTCAGGCTACCCAGCCCT
CGGGAGCTCACAGCAGAGCAGTTGGGGATGCCTTGCAAGGTTACCTGTGCCAGAGTTCCTCGGGTACATATTAAATAGGTAGGACCCAA
TACTACTTTGCATTTTCCTCCAATCCTAGACTCTTAACATTGGAAAGGATTTCTTTGCCAAGTAATTCGCCCTCATCATTAACCTCTAA
GAAATGCAGAGGTGGAATTAGAAGGTGCGTGTCTCTTTCTTTTGCACCACAGATTCTATCGAGTCTCATCTGGCAGCTTCAAGGATCAT
CCGCATTCTTTCATCTTCATGGAATGTCACTAGATTGAGATGATTCTAAGGGTGAGGTCAAGCATTTGGAGTTTGCAACTCTCCCTTTT
GTAGAGATGCTCCCGTGGGACTTGCCCTGCACCCAGTTCAGCTTGCTTGCCATCCATCTGCTGAGCCTGGAAGAGAAGTATGGGTTCAA
GGCCAGCGGTCCAGCACTCCCACAGACCTGATCTGTAATAGCGAACCGTTGCCTCTGTGTGGGTTGGGAGTTTAATCAGCAAGAGTGGA
TTAGCAGCAGGCTCATTCTGTTTACTCCAGTAGGTCACGGGGCCAAGGGCAAACATACACCCCTCCCAGCCCAGAAACTGATAGCTGCA
AAGTTGCACACCCTTCTCCCCAAATTGACAAATATCGTTTTCATTGTTGGAGTAATAAATTATGCTTTTTAACGATAAAAATGTTGCCC
AGCTGTTGAGTGGTTTAAACTTCCAGATGTTCCTCTGCTCAGTAATCAGAGCAGGATGCATGATGGGATCTCTTTAAAAGTTGATCAAC
AGAATGGTTCATTAAATTTCAACAATGTGCCTCGAATGCCAACGCCACGTCTACCATCTCCCTCCACCCAAGGAGGTACACATTTCCTA
GCCTGCTTGCGTCAAGAATTAGCAAGCAGTAACTGAAGACCAGTTACTGGTCAGGCAGGGAAGCCCAAGTTCTGGCCCCTGCTGACTGT
GTGACTTTGGGCTGAGCAGTTGGCCTCTCTGAGTCTTAGGTTTTGTTTTTTTTTTTTCTTTTGTGTTTTTGAGATAGAGTCTCGCTTTG
TCGCCCAGGCTGGAATGCAGTGGCGCGATCTTGGCTCACTGCAACCTCTGCCTCCCAGGTTCAAGTGGTTCTCATGCTTCAGCCTCCCG
AGTAGCTGGATTACAGACATGCACCACCATGCCTAACTAATTTTTGTATTTTTAGTAGAGTTGGGGTTTTACCATGTTGGCCACGCTCC
TCTTAAAGTCCTGGCCTCAAGTGATCCGCCCGCCTTCGTCTCCCAAAGTGTTGGGATTACAGACATGAGCCACTGCCCCGAGCCTGAGC
CTCAGTTTCTCATACTTGCAACTCGGAGCATGTTTTGAGAGCTAGAGAAAGTATTGTTGTCTTGTGTAACATGAAAAAAGCTGTGCGGA
TACTAGCTAAGATCAATGAAAGGTTATCAAACTCAATCTGATGGTGAAAGAAGATACAGAGATCAAGCCCGTCCGCATTTTTAACGTGG
GGACACTGAGCCCCAGGAAACTTGGGCCTTCTTCAAGTAGTACCTACGCCTCTGCTCTCCCATATGGTACCCGCTAGCCACTAGCCGCA
TGTGGCTATTGAGCAGTCGAAATGTGCGAAAATTCAAATTAAAATATGTTGTATATGTAAATTGCACAATGGATTATAAGACTCAGTTT
AAAAAAAAGAAAAAATTCATAACAACTTTTGAAATATTGATTACATGTTGAGATGATAAGATTTCGATATACTAAGTTAAATAAAACAT
TAAAAATGAATTTCACCTTTGGTTTCTGCTTAGTATTTTTTAATGTAGCAACTAGAAAATTAAAAATTACTGCCGGGTGCGGTGGCTCA
CGCCTCTAATCCCACCACTTTGGGAGGCTGAGGTGGGCTGATCACGAGGTCAGAAGATCGAGACCATCCTGGCTAACACGGTGAAACCT
CATCTCTACTAAAAATACACAAAAAATTAGCTGGGCATGGTGGTCGGCGCCTGTAGTCCCAGCTACTGCCGACGCTGAGGCAGGAGAAT
GGCGTGAATCCGGGAGGCGGAGCTTGCAGTGAGCTGAGATCGCACCACTGAACTCCAGCCTGGGCGACAGAGGGTCTCTGTCTCAAAAA
AAAAAAGAAAGAAAATTAACAATTACATGTGTAGTTTGCATTATATTTCCACTGGGCAGTGCTATGCTAAACTGTGACACAAACGGTGG
ACTTTAGAGTTGTGCAGGGTACAATCTGCACGGCCATATGTGGCAGGCCTACCAGGTGTGGATGAGGCTGTCTGGGTTCGAATCCTGGC
TCTACCAGTAATCAGTTGTGTGGTATTGACTAAATTGTTTAAACTCTCAGAGCTTCAGTTGTTCTATCTGTAAAATGAGTATTGCAATA
GTGCATGCTTCATAGAGCTGTTGTAAGGATTAAATGAATTAGTGCATATAAAGTGCCTAGAATAGTGTCTGGCCCGTTTTAAGAGCTCA
GTGAGTCTTGGTTGTTGTAGTTGTTACTTTAGTGGTTGTTTTTTAGAACCTGAGGACACAAGGATGGAAAGACTCAGTCTGTCTGTGGC
AAACCACGAAACGAAAGATTAAACAATTCAATGCAGGCTCGCTCCAGTGCGTCACACTTGTAATCCCACCACTTTGGGAGGCCAAGACG
AGCGGATAGTTGAGATCAGAAGTTCTTGACCAGCCTGGCCACCATGGTGAAATCCCATATCTACTAAAAATACAAAAATTAGCCAGACG
TAGTGGCGAGTGCCTGTAGTCCCAGCTACTCGGGAGACTGAGGCACAAGAATTGCTTGAACCTGGGAGGTGGAGGTTGCAGTGAGCTGA
CATCACCCTACTGCACTCCACCCTGGCCGACAGAGCACGACTCTGTCTCAGAAAACAAAGAAAGAAAAAATAAATTCAATGCAGTCATT
ATTAGAGGGGTAAGTCCATCGGGACAAATCAGGGGGCATTCCCACTGAGGCAGGACTTGAGATGAATCTCGGAAGCAGAAATGCTGAGC
TTGTTGGGCTGGCCAGAGATAAGAAGGACATTGTGGGCGGGAGAAACAGCACCTACAGGGCTGAGATGGATGAAAGCTCAGTTCAGTCT
GGGAACTGCAGAAACTCAGTTTGCCTGAGCAAACTGCAATGTGGCAAGTGGGAAATGAACCCAAGCTAAAAGAGGACAGCACCTGGGTG
TAAATTTTGTCCTGTAACCCATGGAGAGCCAAGAAGGATTTTGAGCCAGGGAGTGACATGATTGGATTTCATGATCAGATCATTCTAAC
TGCTTTGAGGAGGTTGTCTTGGAACGAAAGCAAGAGTCTTTGGCAAGAAGATCAAGAGAAGAAAGATTGCTATTTCCAAAGCTCAAGTA
GTGTATTTTTCATTGTTGTTGTTAAAGTTCAAAAGTCTTTATATTGAAAGTGGCACGTATGCTATGATCTCACACCTGTAATCCCTTCC
CAGTTTATCTGCATGAGAAAAGCGCTTGGAATGGTGCATATCTAAAGGTGGTTTCTGCTCAATGGGGATGTGGATTCTTGTGTCAGGTT
GTTGGGGAGGCTTTAGGCTCTGGCTGCCAGCCCATCCCACTGACACCCAACTTCATATGGATGCAGAGCTTTTTAACCATATCAAAGAG
AAGGATACGAGCTTAAAATATGAAACTCCTGCTTTTGATATGCTTAAATTCTGGGTCTGATATCTTTTTGAAAGAATTAGGTTTTTAAT
TGAAATAGCACATGCATATATTTAACATCAAATGGGTAAGGCTTTTATGCAAAATCCAGATTCCCCTGCCCTTTTTCTGCCTGTCCTTA
GCCCTGTTTCCCAGAGTGAATCAGGGACAGGTCAGTACCTCTAAAGAATAAGCTCATGTTGCTCCTTGTCTGTTGACTTAGACTTCTGG
ACCCTCAGCAATGTCCCGGGACTCACACTGGGGGAGGCAGGGAGACCAGCATGCCCCCTCTTGCCACTTGTACTTCCCATTTTCCCCCT
GCCAAGGTGTGCAACATTTATACTCCCACGCCCACACTTAACACTCCTGTCCTTTATCTGCAAGGTTAATTCCAACAATTGATAAATGG
ATGAACAGTGTTTACATTAAGAGGCTTGTCAATGGCCTTCACTGCCCGTCGAAGCAGTCGGCTTTGATCACATGTCCTTTGTGTTCAGC
TCTCTCTTTGCCCTCAGCTTTCTAATCGCCCTTTAAGGGGCTATCTGACGCTGGGTGAGGTGGCTCACGCCTGTAATCCCAGCAGTTCA
GGAGGCTGAGGCGGGAGGATCTCTTGAGCCCAGCAGTTCAACACTAACCTGGGCAACAAAGCGAGAGCCTGTCTCTACAAAACATTAAA
AAATTAGCCATACATGGTGGAGTGCGCCTGTGGTCCTAGCTGAGGTGAGAGGATTGCTCGAGCCCAGGAGTTTGAGGCTGCAGTGAGCT
GTAATTCCACCACTCCATTCCACCCTGCGTGACAGAGTGAGACCCTATCTCAAAAAAAAGGAACTATCTGGCTTCATCCTAAGAGTAGT
TGCATTTTAAACTCTCAGTCAGCTTCTTTTACTGTAAAATGGGAATAATAATTAACAGTCTAAATTCCTAAAGTGATTATGAAGGCTGA
ACAGGTGATGCCTGTTCCTCAGGCCCCTCCCTTGGGGCCCCTTCAGCCCCATGTGTCCCAGGCTAATCTCAAACTCCTGGCCTCAAGTG
ATCTGCCTACTTTGGCCTCCCCAAGTGTTGGGATTACAGACACGAGCCACCACTCCCAGCCAGCTTGCTATTAAATTTTACCTTTTTAT
CCAGGAGCGTGGGTCATCATTTCTTTCAAATAATGCTTCTATTTATGAGATTTTTCTTTGTAGAAAATGTGGGAAATGTTAAGAGTATA
AGGAACAAAATAAGTCGTGTTAACGCAATTGCTGCATTTTTCTTTTTTTCTTTGCATATTTCTTTTAAACTTGAGATCATGTAATGTCA
ACAAGTTTATTGGATTTTTCTCCTAATGTAGTATTAGTATTTCCATGTGTTGTAAAGAGCAAGCTTTTAGGTATCATTTTTAATGCAAT
ATAATGTTGTAACATCCTATTGCAATGACAGCTTGTCTTTCTCCCAATTTTATCTGCACTATTAGGTATTAAGAATATTTAATGTGGCC
GGGCATGAAGGCTCCTGCCTGTAATCCCACCACTTTGGGACGCCCAGGTGGGTGGAACACTTGAGGTCAGGAGTTCGAGACCAGCTTGG
GCAACATGGTGAAACCGCATCTCCACTCAAAATACAAAAATTAGCTGGGTGTGGTGATGCATGCCTCTAATCCCAGCTACTCGGAAGGC
TAAGGCTGGAAAATCATTTGAACCCAGGAGGTGGAGGTTGCAGTGAGTCGAGATCATGCACTGCACTTCAGCCTGGGTAACAGAGCCAA
ACACTAGCTAAAAAAAAAAAAAAAAAAAAAAAGAACATTGTGTATTTTCCTCTTTTAAATAATGTTGAGATGAGTGTTTTGGTGTATAT
GTCTGTGCATGTGTGCACATATGTGTGCAGAAACCTATAGGATAGATTTCTGCAAGAATTATCAAGTCAAAGGTTCTAGACATTTTTAA
GGCTCTCAATGCATGTTGTTCAGTTGTTTCCCCATTTGGTTACACCAAGTTACAGTCACAAAATTACACCAAATATTTTTTCCTCCATG
GAGTTGCTTTCTCATGAAATTGGGTTTCTGACCCCACGAAGACTTCCGTCTTGTTGAGGTGAAGGGGATAATTCTTCAGCATTTGTCAC
TATTTTTCACTATCATTTTGAGTTTGATTTAAATAGCACAGTTTGCTGCGTGGAAAATGAGAGTAGCTTTGAAGGGGCACAGCTTTGAA
GGGCACACAGTACATGATCACATCTACTTCAGAACTCTGGCTGCTACGTCGGGTACGACCATGTTCCATTTTCAGCATGCTTTACTGCA
AATCAAAAGCATGGGATCCCAATCCAAAACCAAAGGGCTTAGGTTCTGCATCTGACATATCTGGCTAGCTGTATGTTCCTTGCCGCCTC
ACTCAACCTTGCTCAGTACATACCTTCTTCATATGGCGTGCATCTCTTCCTCAGCTATATGGGTGTCCTCGAAAATGCTGAGCATAAGA
AAACTCCATTTCTATCCTCTTTATTTATTTATTATTTATTTATTTTTTGAGATGGGGTCTTACTCTGTTGCCCAACCTGGAGTGCACTC
CCACAATCATGGTTCATTGCAGCCTCAAATTCCTGGGCTCAATGGATTCTCTTGTCTTAGCCTCCTCACTTGCTGGGACTACACCAGTG
TGCCACCATGCCCAGCTTTTTTTTTTTTTTTTTTTTTTTTCATACTAGAGATCAGGTCTTCTTATCTTGCCCAGGCCACTTTCGAACTC
CTTGGCTCAAACAATTCCCCCTCCTCTGCCTCCCAAACTGCTGGGATTATACGCATGAGCTACCGCTACACCCAGCCTATCCTGTTTAC
CTACAACTTGCCAGTAACCTTCCCCAAATTGCTTCGCCTCAGGATCTTCCTTTTGTTTTCTCTAAAATGGGAATAATAATAATAATAAT
AGTCCCTAATTCCTAACGGGATTCCCAAGGTTCAACAAAGTGCTTCCGCTAGATTGCTTGACCTGGTGCCTAATCCTGGTGATTTCTCG
TAAAAATCAGTTGTTCCCACTAGTGGAGTCCACCAGACCTGATCATTCCCCAGCCTTCAGTTCACCCAACCTCCACTTTAGTTCTTCAG
GCTGGTTGTATATGGCTCCCTAGATTCATCGTTTGGAATCTTCACAGTCAGAAGGCACCCATCCAAGTTTGCCACTCAATGGATGACAA
AGTAAGGNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNCCAGTACAGATGTCTGGCATTTTATTGCAGACAAC
ATCTGGGCAGATGACAGCATCTGGTATTTTTATGATTGTCACTTACCAGAGGTATGGATAGACATTTCTTGAAGAAGATAGGAGCAAGC
CTGATTATTTGAGGAACACGAGGGCCAGATAGCAGAAGTGGAGGGAGAGGTGTGGTTGCCTTGGCAGGTGGAGAGGGTCAGCCATGGAA
GCAAGGAGCTTGCTATGTGCCCGTGTGGGACTGGCGTTGGGGAAACCATATTGCTTTATTTCAGGAGAGAGTGATGTGCCCTGTGTCTT
GGTGGTAGCAAAGAAAGCGGAGAGAAAAACGTAGATTTTACATTTATCTGGGCAATTGTGCCAATAGGACTTGCACACGGATTGGATAT
GGAGGTGAGAAAATAAGGGAATCAAGAATGACTCCTACGAGTCAGGCTGGGTGGTTCATGCCTACTATAGTCCTAGCACGTTCCGGGGC
TGTGATCGTTAATACTGAGTGTCAACTTGATTGCATTGAAGGATGCAAAGTGTTGTTTCCAGGTGTGTCTGTGAGGGTGTTGCCAAAGG
AGATTAACATTTGAGTCAGTGGGCTGGGGAAGGCAGACCCACCCTTAAGCTGGTGGGCACAATCTAATCAGCTGCCAGTGAATATAAAG
CAGGCAGAAAAACATGAAAATGCAAGACTGGCCTAGCCTCTTAGCCTACATCTTTCTCCCATACTGGATCCTTCCTGCCCTCAGACATC
GGACTCCAAGTTCTTCAGTTTTGAGGCTTGGACTGGCTCTCCTTCCCCCTCAAACTTGCAGACAGACTATTGTGGGACCTTGTGATCGT
ATAAGTTAATATTTAATAAACTCCTCTTTATATACATACGTGTGTGTGTGTGTGTGTGTGTATATATATATAGATAAAACTCCTCTTTA
TATACATACGTGTGTGTGTGTATGTATATATCTATATCTATATCTATCTCTCTCTCTCTCTCTATATATATATATATCTCCTATTAGTT
CTGTCCCTCCAGGGAACCCTCACTAATACAGAGGCTGACCCACGACCGCCGTTTGAGGTCAGGAGTTCAACACCAGTCTGGACAACTTA
GAGAGACCTTGTCTCTACAAAAAATTAAAAAAAAAAAAGAATGACTCCTAGGTTTTTGGCATCAGCAACCTGGCAGATGCTGGTGTCTT
CAATGGACACGGGGAAGATGGGCAGTAGATTGTGGGCCCAGAGAGTTCTGTGCAGGCATGTTAAATTGCACATCCCCATTAATGGCCAC
AGGACATGCCAACTACGTACTTGGCAAAGTCAGTCTGCCAATTGAGACCATCATGGTAGGAGATAGCATTTGGAAGATTTTGGCAGAGG
TGGGATCCAAATCCATGGGAGTGGAGTATAGATAGAAAAGTGGACATACCACTAGACATGCCCACTAGTGCTAGCGGTACAACTTGCGG
AGCTGGAAAAGGTCATTACAACGAGCCACTGCTCAGGCAGAAAAACATGCCCCCAGCCTAGACAAGGAAGACCTTCTTAAGGAAGGAGG
AGAGTCATCACTTGCAATGTTGCTAAGAGGTCGGGTAGAATGAGTCACACAGAAGTGACTGTTAGATTTGGCATCAAGGAGGTTGTTGA
CTAACTTGGCAAGAGCGCCTCATGGGAGCAATCAAGAGCAAGCCAGTGACCAATCAACGTGACTCCTCTTGCCTTCTGCTCTCTCTTCA
TTGCCTTTTGCTCTTTGCTTAGGCAAATCAGTGTCTTCAGTAGGTTCCTTGAAGGCTAATGTCACCATTAGTATTTACCTACCCATTAG
CTCATTTCATTCTCAAAACAACATCTTTGCAGAAAAGGCTGGGCAGCGCAGGCACCTATTATTCCCATTTTACAGATCACAAACCCACA
ACTTAAAAACGTCAAGTGTTTTTTTTTCAGCCTGTTTCTTAAAACTAAGACTTATGTGTCACCCACCCCTCCTCCACCCCCATCTCCCC
ATACATCCCAGTGTTTTGTGGTTTGCTACATTTCTTGGTTTTCTTACCTAGATTGTGAACCCAGTCAAGCGACGATCTGAATCACATCT
CTATATTCCCACGCAGAGATAGCAAATGCTTGCCACACTTGTTACTTTCTCTTGGCTACCCATCCCAGGCATCACTAGCTGATCATGAC
CTTCTCTTCCTTTGAGCCCAGACTCACCTTAGAATTCTACCCAGCATTCCACATACCCTCTCAACCTGCTGACACATAACCTATCATCA
ACACCCATTTTTACTGTAGTACTTAATGTCATGCCTGCTTCAGCAAGTGGTTTTGTGACTGATTTAGCAAATAACCATTCTCTCAAAAC
ACAGTGAGTGAGTTCTATGTCACTACACCTAATCAAGAAGAGATCAAATTCATCCACATGCCCGGGTGTGGTGGCTCACGCCTGTAATC
CCAGCACTTTGGCAGGCCAACGCGGCCGGATCACGAGGTCACCAGATACACACCATCCTCCCTAACATGGTGAAACCCCATCTCTACTA
AAAATACAAAAAATTAGCCGGGCGTGGTGGCGGGTGCCTGTAGTCCCAGCTACTTGGGAGGCTGAGGCAGGAGAATGGTGTGAACCCGG
GAGCCGGAGCTTGCAGTCACCTCAGATCCCACCACTCCACTCCAGCCTGGGCGACACAGTCAGACTCTGTTTCAAAAGAAAAAAAAAAA
ATTCTTCCACAATTGGATAAGGTAAATAGAATTCAGGTATTGGGTAAAAGGTTATCCAACTCTGAGATTATCTCATATTCATCGAGTTA
TTTAATGAACAGTAGTATCTCTTACCATTCTGGAACAAAACAAGAAACCTTTGAGAGGAATCTCCCCCCACCTCCCCAGATATATAGGA
GGGGACAGTGGCCAACACTATTATGGCTCCTTTCTTGTTGTCTTCCCCACTTCGGCCTCTCCTCTCAGCCTTTCTCCATTGCCATAATC
TGTTGGGCCCTATGGAGCCAAAGGGAGTAAACAGAATAGTACTGTTCTGGGTTGTTGAGGGATTCGGTGAGATTCCCTAGGTAGGTTTT
CGTCAACAAGCAGGCATTTCATAATCCTCTCTACCTCCTGCCCTGCCCTGGGGATTGTGACGATTGTCTGCATTCCTCTGTGGAACTGG
AATGTCGCTGCAGTGATGGTGTCTGCAGAGATGTTGAAGCTGCTCCTCTCTCCTGCCCTCTATCTTGGCCTCGCCCGGTCCTAGCCGGG
GTAAGTGAACATCAATCCCATGCACAGAGTCTTCTCTGAAAACTCCTTTATGGATTTTTTTTTTTTTCCCAGACAGGGTCTTCCTCTCT
CCCCCACGCTCGAGTGCACTGGCACAATCTCACTGCAACCTCACCTTCCTCGCCTTAAGTAATCCTCCCACCTCACCCACCCCAGTACT
TGGGACTACAGGTGCATGCTATGATGCCCAGCTAATTTTTGTATCTTTAGTAGAGACAAGGTTTCACCATGTTGCCCAGGCTGGTCTTG
AACTCCTGGACTCAAGCGATCTACCTCCCTCAGCCTCCCAAAGTGCTGGGATTACAAGCCTGCCTTATGGATCTTAATGTCTTGGCCCG
AGGGCATTGGCTCTGCCTGGCTCCAGAGGGATATGAAGCCATTCCTGGGGGACACCATTGTTTTCTAGTTAATATTATCAGTCCTGTAG
TCCAGTGATTTTCAAATTTTAGTGTAGTCAGAATCACCAGAAGGGCTTGTTCAAACCCAGACAGCTGGACCCCTCCCTAGAGACTTAGG
ATCAGCCCTCAGAATTGGATCTAACAAGCTCACAGGTGACACTGAAGTTGACGACCTTGTATCTCACTTTAAGCAAAGTCTTCCCATAG
GAACCATTGTTCCTCCCTGCCCATAGGTAATATCTTCTACTTGGGCCCTCTGCTTCCACTGTGGCCTGCTTACAACCTCACCTACACTT
ATTAACTAGCAGAGTTCACTGAGTCTGCTTAAAACCTTAAACTTCAAACTATACCACAAGGCTACACTAGCCAAAACACCATGATACTA
GTACCAAAACAGAGATATAGACCAATGGAACAGAATGGAGGCCTCAGAAATAATACCACACATCTACAACCATCTGAACTTTGACAAAC
CTGACAAAAACAAGCAATGGGGAAAGAATTCCCTATTTAATAATGGTGTCAGGAAAACCGGCTAGCCATATGTAGAAAACTGAAACTGG
ACCCCTTCCTTATACCTTATACAAAAATTAACTCAAGATGGATTAAAGGCTTAAACATAAGACCTAAAACCATAAAAACCCTAGATGAA
AACCTAGGCAACACCATTCAGGACATAGGCATGGGCAAAGACTTCATGACTAAAACACCAAAAGCAATGGCAACAAAAGCCAGAATTGA
CAAATGGCATCTAATTAAACTAAAGCGCTTCTGCACAGCAAAAGAAACTACTATCACAGTCAACAACGAACCTACAGAATCGGAGAAAA
TTTTTGCAATCTATCCATCTGACCAATGGCTAATATTCAGAATCTACAAAGAACTTAAATTTACAGGGGAAAAAACAAACAAACAAACA
AACAAAAACCCCATCAAAAAGTGGGCAAAGGATGTGAACAGACACTTCTCAAAAGAAGACATTTATATGGCCAACAAACAAATGAAAAA
CAGAAAACAAAAAACAACTCATCATTACTGCTCATTACACAAATGCAATCAAAACCACAATGAGATACCATCTCATGCCACTTAGAATG
GTGATCATTAAAAAGTCAGGGAACAACAGATGCTGGAGAGGATGTGGAGAAATAGGGACGCTTTTACACTGTTGGTGGGAATGTAAATT
AGTTCAACCATTGTGGAAGACAGTCGGGTGATTCCTCAACGATCTAGAACCAGAAATACCATTTGACCCAGCAATCCAATTACTGGGTA
TATACCCAAAAGATTATAAATCATTCTACTATAAACACACATGCACATATATGTTTATAGCAGCACTTTTTACAATAACAAAGACCTGG
AACCAACCCAAATGCCCATCACTGATAGACTGGATAAAGAAAATGTGGCACATATACACCATGGAATACTATGCAGCCATAAAAAAGAA
TGCATTCATGTCCTTTGCAGGGACTTGGATGAAGCTGCAAACCATCATTCTCAGCAAATTAACACAGGAACGAAAAACCAAACACAGCA
TGTTCTCATTCAGAAGTGGGAGTTGAACAATGAGAACATATGGACACAGGGAGGGGAACATCACACACTGGGGCCTGTTGGGGGATGGG
GGGCAAGGGGAGCGATGGCATTAGGAGAAATACCCAGTGCATGCGGGGCTTAAAACCTAGAAGATGGGTTGATGGGTGCAGCAAACCAC
CATGCCACACGCATATCTATGTAACAAACCTGCGTCTTCTGTACATGTATCCCACAACTTAAAGTACAAACAAACAAACAAAAAACCTC
CACAAAAACCCTATCTCCAGGCTTCCCATTGTATCTCCTGACTTTTTTGAAATCCTGAGCAGCCTGTTCCTGTTGATCGAGTGAATGAG
CATTCTCTGAAATTGCAGTGAGTGACTTCTCCATCACTAGAGGTAGTCAAGAAGAGGTTGCAATCACACACAATTGGATGAGGTAAATA
GAATTCATTTGTTAGATAGACTATGCAGGCCTAAGATTGTTTAGTATTCACGGAGTTATTTTGTGAACACTAGTGTCTTCCAGCCTCAT
CAAGTGGTCCCTCCCTCTGTAGCCCCACATCCTGGTCTTTGATCAGTTCCCTGACACCCTTCCCTGGGCACCACAGGACCTTTGCACTT
GCTGGTCCCACATCCTAGAATGCTTTTTCCCCACCTTGTTGAAAAGCTGATTCTCATCCTAACTTCAATATCACCTCCTCAGACCACCC
CTTCTAATAGACTCATTCCCTAACCTCCATTTCTCATCACTGGATGCTGCTGAATTTCACTGTGGCACCAGTCATAGTCTGTAAAAAAA
ATTTATTAAATTATTTGGCTCTTTGATTTTGGTGTGACTCCTCCCCTAGAGAAAAGAAACTGACTTTGTCCTTGAACTCTGTCTTGTCT
GACACTCAGTGGGTGGTTCATACATATTCTTTTTTTAAAATTAATTTTAATTTTTATTTTACATTCCGAGGTACATGTGCAGGATGTGC
AGATTTGTTGCATAGGTAAACGTGTGCCATGGTGGTTTGCTGCACCTATCAACCCATCACCTAGTTATTAAGCCCAGCATGCATTAGCT
ATTTTTTCTATGAAAGGTGAGCAAATGCTTGTATACAGCCTGCACAGGGTTACGTCAAGGCTGAACCATGTACTCTAAATTTTGGACTG
ACTGCAGTGAAGCCCCTCCATGGTGGAAGAAAATGGAATGTCCTTGTTGGAGCTGGCCAGAACTGGGCAGATCCAGAGGCTGGAGGTAA
TCCCAATAACTCTGACTGCTAATAATAAAATAAGAACGTAAAACACTGCTTACAGTGTGTTACAGGCACTGCTTTAAGTGCATTACCTC
ATTTAGTTTCCACAGTTGTCTCCATGTTACAGAGGAGAGGACTGAGGCACAGAGTATTACAATAAGAAACTTGGCCCACAGTCAGCGAG
CAAGAAAGCCTAGATTCATACCTGGGCATATGGCTTCAGAGTCCACACGCTTGGCGCCTGCACTGCACCGAGAGGGGCTGCTGGGCCGG
GAGTCCCCCACCTGCTCTCTGTTTTCAAACTCCCCATTTCACACCCATGCCAGGTGTGATGAAATCCTCTAGGTGATGGGCAGTGGACA
TGAGTGGTTCTGGTTACAGTTGGACTTCACTGGCCTGTCCCACACCTGAGGATATGGATTCTAGCCCATGGGACCATAAGCCAGGCCCC
CTGAGGGTCTGTTATTTGTCTGGGAAGTGGCAGCGCTCTTTGGAATCTTGTTCATGACTGACAAACACCCCCACGCCCCTTCAGCCATA
ATTATCACCTAGTCCCCACAGAAGCGCCATGCGTCCGCTGACTGTAGAGATAGACGCTTGCGTCTCTGGCTGGGAACACCACATGCAAG
AGAAGAGGCCATGGGCTCGCCTTCCATCCAGCTGAAGTCTTTCTGGCCGGGCTGCAGGCTTCATGCAACACCTTTCAACCTGTAAAAGG
AAAAAAGAAGGCCGGGGGTGGGCCTCAGCTGCTGCCACTGGCATTGTATCACTGAAGCGTTTTTTGTTGTTGTTTTTTTTTTTTTGAAT
ACCGGAAATTAAGGGGAAATTCGGAAACCTCATAATGCCTTCATGAACCATTGCACTTGGAAAATGTACTTTTGAAAAATTCCCTTGAT
TTATTGTTTTTCGGAGGCTGTAGATAACATTAGAGTATATTATTCCTATATAATTTCCTGAGTTTTCTATGTCAATTACTTTGTTTTAT
TAAAGAGAAAAAAGGAACTAATATTGATTGAGGCCCTCCTGTGTGCCAGGCTAAANNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNTTGCAAGCCT
ATCGACACATACTGGTAATCGAACCTGTGCTCACAATTGTTGGGAATCTCTAGTTTAAAGCTTTGTCATTTGGAGGAATATCAAGTAAA
CCCTATGGGGTCAAAAACAAGGTCTAGCCAGGTGCGGTGGTTCATGCCTGTAATTCCAGCATTTTGGGAGGCTGAGGCGAGTGGATTAC
CTGACGTCAGGAGTTCAAGACCAGCCTGGCCAACATGGTGAAACCCCGTTTCTACTAAAAATACAAAACTTAGCTGCCTCTGCTGGCAG
GCACCTCTAATCCCAGCTACTCACCAACCTCAAACAGGAGAATCACTTCAACTCGGGAGGTGGAAGTTTCAGTCAGTCGAGATGGTGCC
ACTGGACTCCAACCTGGTCAACAGAGTGAGCCTGACTCTGATGATAGTAATAAATAATAATGGTAGCTTCTATTTATTGTTTACTACTA
TCTCCCAGACACTATTCTAGCTGCTTTTCATCAATTAGCTCATTTCATCTTCCTTATAACCCTATGTTCTAAGTACAGTCATGTACCAC
ATAATGATGTGTGGATCAGCAATGGACCGTATATACAACCGTGCCCCTATGACATTATAATACAGTATTTTTACTGTACTTCTTCTATG
TTTAGATATGATTAGATACACAAATGCTCACCATTGTATTAAGTTGACTCCGATGTTCAGTACAGTAACATCCCATTCAGGACTGCACC
CTAGAACCACTACGCTGTACCATATACTCCACATGTGTAGTGAGTACCCTGTCATCTAGTTTTGTACAAACTCCCTCTGTAATGTTCGC
ACAACGAAATTGCCTAACGCCGCATTTCTCAGAATGTATCTCTACCCTTAAGCAACGTGTGACTGTCCTATTATCATCACCCCATTTTA
CAGATGAAGGAGCTCTGCCAAGAAATTGACTAACTTCCCCCAACGTCACCCATATAATAAAACGCGCGCTGCGTGCAGTGGCTCGTGCC
TCTAATTCCAGCACTTTCGGAGTCCAAGCGGAATAGGATCACTTCACCAGACCACTTTGACACCACCCTGGGCAACAGAGGCAGACCCT
ACCTCTACCAAAATTATAAAAAAATTAGCCGGGCATGGTGGCATAGACTTGTGGTCCCAGCTGCCCAGAAGGCTGAAGTGGAAGGATGG
CTTGAGCCTGGGAGGTCAAGCCTGCAGTGAGCCGTGATTGTGCCACTGCACTCCAGCCTGGGTGAGAGAGTGAGATCCTATCTCAAAAA
AAAAAAATAAAAAAAAAATGAAAAAAAAAATAAAAAAAAATAAATAAAATAATTAAAAAAAGAGCTGGGACTTCAAGCCAGATAATAAG
CCAGGCAGAGGGCATACGAAACACCCAGATGGGTCATGAGCCCCTGCATCAGTCAGGGTCTAATCGCTCAGCTGAGGAAGGGAGGTTTA
AGGAAGAGACTGTGGACAGAGGTGTTAGGGAAGGTGTCAGAGAAGGTTAAGGAGCCAACATGGATCATCGGGGTGCTACACTCTTCCCA
GGGCTGGGGAGGATTGGCTGCAGTGTGGGGTACCCAGCCGCTGCCATGTGGAGAGGGACCTGTCACTCCTGCTGTGAACTCTCCCTTCT
TCTCCCCTCTGACCTCCTGCTGGTGCCTCCCATTGGCTAAACACAGTTGATCGCCAGTCCACTGGGCAGCTGTTCTTGGAGCCCACACG
CATCTGCTTCTTGGCACAGAGCAGACAATGGATTGAGTCGGGAGGGAGGGGAACTAGAGAATACCCAAGTCCCAATGGCTGGAGAGGAG
TCACCAGCTAAACGGCCTAGAAGGTCAAGTCAGAGACGCCGCCGTCCACACTACACAGGGCCTTTGGCTTTCTTCGGAAGGAGCAGGAA
GCCAGTGGAGGGTTTGAGCAGATGTGTGCATCACGGCCACTGCCATCAGGAGAACGTCTTTCTCTATGCTAGGTGTGAACAGTCACCCC
TTCCTCTCCCTTTCTCAGGATGAGTGCTGAGGAGGAAAAGGCCCTTTCCTCCAATGTCTTCAGAGGAGACGTCTGCCCAGTCCAGACTA
CGTGCCTCATGCCAGCCAGCCTGTAAATGACAGGAAAGAGAAAGGAGCAGTGTCTCAAAAGAGGATATTCTCGAACGTTTGATAATCAG
AGCCCATTGTAAAGTCATGTTTTGAGTTACAGCATTTAAATCTTTGTTCCAGTGGAAAACTTTTACAGGCCGTCCTGGGCAAGGCTTGA
ATCCTGTTTGGATCTCCAAAGGCCTTAAAATACTCTGGCATGCCGGGAGACTGGCTCCAAAAGGGAGGAGACCTGTGCTGGCAGCCCTG
GCCACACCTTGGTGGGACCGGCTCCTGGCCTTGGGGACCCTGCCACTCTCCTAGCTGTGCTTCCGCAGAGCTGGACACACAAAAGCTTT
CTTGTCTGGATTTGTTCCCCTAAGAGGGTGGCACCCAACTCGTCCTGCCCATGCAGGAAGCTGGTCCTGTCCAACCAGGGAATGAAAAG
TTGGCCAGTATCTTCTCAGGCCACGCTACAGCCAAGTCTCTTTTAATGAGCTGGTTCTCTTAGGATGCCCAGGGCAGGCAGAACACACT
TGAGTGGGATGGCGAATGAAGAAGGAAGCGGCTGTTCAGACAGCTTCCAGTTCTGTCAGGTTCTGCTGCTGTGACCAAGGCACCAAGTC
ATCTGGAAACCCACTGCAGGCAGCAACCTCTTTTTCACCTTTGTGTGCCCCAAAGCTTGCTCCGGGAATCGATGGTTTGGGAAACAATG
CTTGCTGTCTTTGAGTCCTCAGTCCTGTAAAATGGGGAAAGTATACATCAGAGGGCATTTGAGAGTAAGGCAAGAAGCGCTTGGCATGT
GCCTGGCACATAGTACCTGTTCAGCAGATTCCCTTTCAGCTCAGGGTTAGATCTTCATCCACCTCTTGTTGGCCTGAAGGAAGGGGAGG
TAGCTACTATTAGCCCCATTTATTTTATTTTATTTTTTTATTTTTCTTGAGACAGTCTTGCTCTATCACCCAGGCTGGAGTGCAGTAGT
GCCATCTTGGCTCACTGCAACCTTCACCTCCCAGGTTCAAGTGATCTTCCCGCCTCAGCCTCTCAAGTAGCTGGGATTACAGGCATGCA
CCACCACGCTTGGCTAATTTTTGTATTTTTAGTAGACAGGGGGTTTTGCCATGTTGGCCAGTCTGGTCTTGAACTCCTGACCTCAAGTG
ATCTGCCCACTTCAGCGAGATTATAGCCGTGAGCCACCACATGTGCCTTATTAGCTCCATTTTATACCAGGTGAAATTAGACAGCAAGA
GGCTAAATGCCATGGCTGAGGTAAGATCTGTGGTAGCATCCAAGTCTTGACCCCAGACTTGGTGTACTTCTCATCACAGATGCAATTTT
GTGCCTCATCATCTGACCCACGAGCTACACATCTGCTTTTTTTTTTCTTCTTTCTGAGACAGTCTCACTCTGTCACCCACGCTGGAGTG
CAGTGGCACAATCTTGGCTCACTGCAACCTCTGCCTCCCAGGTTCAAGTGATTCTCATGCCTCAGCCTCCCAAGTAGCTGCGACCACAC
GTGTGCGCCACTACCCCCAGCTAATTTTTGTCTTTTTAGTAGAGACGGGGTTTCACCGTGTTGGCCAGGCTGGTCTTGAACTCCTGACC
TTGTGATCCACCCTCCTCGGCCTCCCAAAGTCCTTGGATTACAGGCGTGAGCCACTGCGCCTGGCTAAAGTTGTTATTTTAAAATTATT
ATTTATTTATATTTGTTATTATATTTTTTATTTATATATATTTTGTTATCTTATTTTTAAAAGTTGTATGGCCCATTTAAACTTTTGCA
GAAATAGTTTTTTGAATCCATGGATTTAATGCTTTCCTCTTTAAATTTTTTTTAAGTCAATTTAGAGTTTTTAACACAAAAACTGGAAA
ACGGCTTTGCTCTTTTGTGGAACTGCTCTGGATTTTTTTTTTTTTTTAAGAAAGAATTTTGCTCCATCACCCAGCCTGGAGTCCACAAC
TACAATCATATCTCACTGCAGCTTTCAACTCCTGGGATCCAGTGATCCTCTTGCCTCAGCTTCCTGAATAGCTGGGAATACAGGTGTGC
GCTACCCCACCAGGCTATTTTTTTTTTAATTTAAATTTTTTTTGTAGAGATAGGGTCTCTCTTTGTTGCTCAGGCTGGTCTTGAACTCC
TGGCTTCAAGCAATCCTACCACCTCAGCCTCCCAGCGAGTTGAGATTAAAGGTGTGAGCCACTGTGCATGGCCTGTGTTGAATTTTTTG
TCTCCCCATTCCCAAGTAGCAACTCTGGACTCCACAGAAAGGCCGAGTCTCTCCAAGACCCAGCCGTCTTGGAGGGTCATCTGTCCACT
CCTCCAAAGAGGGTGAGACAGGGCCAATAATGAAGTGATCACTGTGCACCAACCCTCCCTCTTGCTTCACAAGACACCTTTTCATCCCT
GAGCCAATTAATCAATTTCAAGAGATAGCATCTGAGTGAGATGGGTCACCAAGTCACAGAGGCCCTCAGCTCCTGTGCCGGCTCTCAAA
CGGGCTTCAATGAGGTGGCCTTAACACATTGCCTAGTACCCAACTTCCTAGAGTTCGTATAGTAGAGAGCAAGTAGCATAGGGCTAGAA
GGCCAGGTCACAATACCCTCTCTGGAGTCTTCTGAATGTATGACCAATAGGCAAGAGCCCCAGTTTCCTGGTCTACAGAAGTGACAGAT
GGGACCCTACCTACCACACAGGTTCTCAGGATAAAAATAGTGATATGGGCTGGGCACGGTGGCTCGTGCCTGTAATCTCAGCACTTTGG
GAGGCTCAGGCGGGTGGATCACTTGAGGTCAGGAGTTCGAGACCAACCTCGCCAACATGGCAAAACCCCATTTCTACTAAAAATACAAA
AATTAGCTGAGTGTGATGGTGCATGCCTGTAATCCCAGCTACTCAGGAGGGAACTGCTTGAACCCGGGAGGTGCAGGTTGCGGTGAGCT
GGCATCATGCCACTGCACTCCAGCTTGCGCAACAGAGCGAGACTCTGTCTCAAAAAACAAACAAACAACAAAACAGGTAATATGGTTGG
AAGAGGTTTATCCTTGCCAAGTTTTTGTAAATCAAACAGCTCTTGAGAGATGCCTGTTTGCCTGTTTTTATTTTTTTAGAGATGGAGTC
TTGCTCAGTTGCCCAGGCTGGAGTGCAGTGGCGCGGTCTCGGCTCACTGTAAGCTCCGCTTCCCGGGTTCACGCCATTCTCCTCCCTCA
GCCTCTTGAGTAGCTGGGACTACAGGCGCCCGCCACCACGACCGGCTAATTTTTTTTTGTATTTTTAGTGGAGACGCGGTTTCACCATG
TTAGCCAGGAGGGTCTCGATCTCCTGACCTTGTGATCCACCCTCTCCCGCCTCCCAAAGTGCTCGCATTACACGCCTGACCCACCACGC
CTGGCCTGTTTGGCTGTTTTTTTAATAGGAGGTGGGAATCCCTTGTCTTCCTGGGCAGACTAGATGTGATGTGTTCACAGGGAGCTTTG
TACAGATTGCTTGAGAGAAGCACCTTTCTTTCCAGAGTGGCTCCTTGGACGATGCCTTCCAAAGCTGCTAAGTCCTGTGGCAGGGGATT
GTTGATATTCTTCATGGTTTCTCTGCGGAGCCTCCTACCCTCTGAATTATTGATGCATGTCTACATTATTATTATTTTTTTTTTGAGAC
GGAGTCTTGCTCTGTCACCCAGGCTGTAGTGCAGTCGCGCAATCTCGGCTCACTGCAAGCTCTGCCTCCCGGGTTCATGCCGTTCTCCT
GCCTCAGCCTCCTGAGTAGCTGGGACTACAGGCCCCCACCACCATGCCCGGCTAATTTTTTTTGTATTTTTAGTAGAGTTGGGGTTTCA
CCGTGTTAGCCAGGATGGTCTTGATCTCCTGGCATGTCTACATTATTAAAAGGGTCTCTTTGCATGAGTTAGCAGAATGTAGTGCCCTC
TCGTGAATGAGAAAAGCCGCCCAGTCCTGATTGCAGCCTCATAATGGGTCTGAAATGACAAGTGTGAAGCAGTGGGCAGAGCCAAGACC
ACCTTTATTTGTGCATCCGTGCTCTTGGGTGGGTATGTTTCACACAGTTTGTTGTCCTTCTCTCCCTTGCCCATGAGGCCTTACAAACT
CAGCTTGTCTCCACCCATTCCTCTACAACCCACACAACAACTTGTAACCTTTTAATCTTCTTTGCTTCCTAAAGATGCAACTCATGCCA
CCTCCCCCAGGAAGTTCTCCCTGACTTCCAATCTTTACAATAGGCTGCCAGACATCCCTGTGTTAACTGTCAACAGAGCCCTTTTCATC
CTCTTGCATCACAATTCCTGTCTCATCCACTCGACTGAACTTCTTGGGTAGGAATGCAGCTCTGTTTTTTTTTTGTTTTCTTTTTGTTT
TTTTGTTTGTTTTTAAGATGGACTCTCACTCTGTTGCCCAGGCTGGAGTGCAGTGGCACGATCTCGCCTCACTGCAACCTCCGCCTCCT
GCGTTCATGCCATTCTCCTCCCTCACCCTCCCCACTAGCTGGAACTACAGGCGCCCACCACCACGCCTGGTTAATTTTCTGTATTTTTA
GTACAGACGGCGTTTCACTGTGTTAGCCAGGATGGTCTCGATCTCCTGACCTCGTGATCCACCTGCCTCCCCCTCCCAAAGTGCTCGGA
TTACAGGCGTGAGCTACCACCCCCGCCCGCAGGTGTGTTTTACATGCATCTCACTCCCTCCGACACCCCCTGGAACCTACCACCTGTTT
TATCTCTGGGGGCTGTTGGTTGTAGTACTGCGGAAAGGTTTCTTAAATGAGGTGTGTTTAGAATTTAGCCCTTTGCAGGGTATAAAATC
TGAGAAATAAGGTGTGTGTTGTGACGAGGGATGGCACTGGGGGCCTGCCTAAACCACTGGCTTCATTGTGGGCGCTGAGGTGCCTTGTT
TCCTTGGAGCTAATGGTCAGAGTGAGGAGCTGGCGACTGAGCTTGGATCTAGATTATTCTTGCATCAATCGCCTAAATCAACACTGTTT
TCTCAACTCCCTATAGAAGAGGGAGATCCTCATGGCTTTCAAGGAGTCATGCCCAAGATACAGGAATGGCTCTCTGGTGGACTGGCTCG
GACAGAGGGGAGAGGGTGAAAGTGGGGAGGACGAGGAAGTTTAACAGTATTTGAAGCATTTGAAAGCCTAGACCCATCGTACGCTGAAA
CAGAGAAGAATGGCTAGCCAAAAATACTCTGTCAGTCTAGCAACTTAAGAATGCTAGTCCTTGACCCCAAACATACTTATATAGCAGAC
TCAACATTATTGTCTTCCACCTACCGGATGGGATTATGCAAGTGATATGACCCAGAGCATTCTGGAAGCCGCCAGGGGCATAAATAATA
CATTAGATATGCAGATGAACTCCCCAGGGTGTCTCTGGTCTTGGGAACAATTCTTAGTAGACCTGACATAATTGCAGAACAGAGTGTCC
TGTTTTCCCATCCACAGCCTCCAGGCCTGGCGAGGATTTTGTGTTCAAAAGAGATTTCCAAGGTGGACATCTCTCTGACACTGGCGATT
TTTCTCTCTCACTTGAACAAAACTCGGTGGAAAGGTCACTCCTATTTGTCTTAGCTTCTCTGTCACATCCCACCCACACTTTCAAAGGT
CTGTTCTTAGCGGGGTCCCAGGAACGCTGTGCTCGCCCTGTCACCCCTGAGCTGGGTGGTAGTCCTTTATCAGAGATACCCATGAGAAT
CACTCACCCTGGAGNNNNNNNNNNNNNNNNNNNNGTGACTGGAGCTTTAAGAGCTACTGAACTAACTGGTAATAAAAGTGAACAAAGTA
ACCAGTCTGGAAATGTCAGTAGAATGGAAAGAAAGAACAGCTGGGAGACTTCTCCATTTTTTCACGGTAAGACCCGGTTACTTGGCTTT
TGTTGAGTCCCTGGTGGGTGTCCATGGTGTCTTTGGCCTTATCTCCCTACTAAACAGGATTACCTGTGTGGGGCACCTTTCCTGGTCTC
CTCTCTCTCCAAGGAGCAGGTAGTACTTCGAGGCCGTGGTTAATCACATTTCCTAAGTGAGCTTTTCCTTTCTACAATAATCCTTGCTC
TTACCCTTGCATTCCTACTGCATGTTCCCTCAGGCTGAAGCCTTCCTTTACATGTGAGCATTCTCCTGGATCAACTCCCTGATGACTGG
AGCCTGTGCCGTGTGTTTCCAAGTTGCACCCACCACCCACGGAGACTAGCTCATGGCCGAGTGGCAGCTGGTGTAGCTGATAGCTGTCT
GGGAAATGGGTCTGCACAATATAAAGACAGCTCACAGCAAAGCGCCCCCAAATGGTACTCTCTTTTTTTTTGAGATGTTGCCCAGGCTG
GAGTGCAGTGGCGCAATCTCGGCTCACTGCAACCTCTGCCTTCCGGATTCAAGCGATTCTCCTGCCTCACCCTCCCAAGTAGCTGGGAT
TACAGGTGCCTGCCACCACACCCAGCAAATTTTTTTTTTTTTTGTATTTTTGGTAGAGATGGCGTTTCGCCATTTGGGACAGGCTGGTC
TTGAACTCCTGTCCTCAGGTGATCCGCCCACCTCGGCCTCCCAAAGTGCTGGGATTACAAGCGTGAGCCATTGCACATGGCCTTTTTTT
TTTTTTTTTTTTTTTTTGTTGTTGTTGTTGTTGAGAAGGAGTATTGCTTTGTTGCCCAGGCTGGAGTGCAGTGGTGAGATCTCAGCTCA
CTGCAACCTCCGCTTCCCAGGTTCAAGCAATTCTCCTGCCTCAGCCTCTGGAGTGGCTAGGATTATAGGCGCCCACCACCACACCTGGC
TAATTTTTGTACTTTTAGTAGAGACAGGGTTTCACCATGTTGGCCAGGATAGTCGAACTCTTGGCCACCGCGCCTGGCCCCAAATGACA
CTTTGATAAGTTGCTCTACCCAATCCTAAAAAAGGAAATACAGATAAAAAATACCAACGTTGTATAATACACATGTTGTTGATGCGCTC
TGTTGTTGCCAAAAGCTCTGTTGCTGGACGGAATGCAAGTGGATGTGGCCCTTTGGGAAGAATTATGGCCTTTTGGCATTCTCTATCCA
AACTACAAAAGCATGTGCCCTTTGACCTGGCCATCTCATGTTGGGACATTGGGATATTTCCCCTACAGAGATAACCTATAGATATTTCT
AGGAAAAGAACAGTGCCCAAGGTTATTCATGGAACATTTTTATAAAAGCAAAAGACTGGGAGTAACTCAAACATCTGTGAGTTGAAGAC
TGGCAAATGATGACACATTGTGCTAGGCAAGATGCGGAAAGGAATAAGGAAGTGCTGTGTGCACAAACACGGGGAGATCTCAGGATGTA
TTGTTGACACAAAAAGCACAATGTGGAGCGGGGAGTATGGCATGCCATCTTTTCTGTAAAAAAAAAGCATGGGTGTAGAGGGGATGTAA
AAATGTGTCTTTGCACAAACTCTGTTAGGATGCACAGGAAACCAACACAAGTGGTGGCCTGTGCGTGGTGGGAGGAGGGAGATGCAGGG
AGGCGAGACTGGGTTAGGGAGGAAGATGCATGAAGAAGATTTTTTTTTTCTTTTCAGAGATAAGGTTTCACTCTGTGGCCCAGGCTGGA
GTGCAGTGGTGTGATCATAGCTTGCTCCAACCTCAGACTATGAAGGAGACTCTTGATTGCGTATCTTTGGGTATCATTTGAATTTTGAA
GCATGAATATATTGAAATCGGCAGCAGCTCAAACATTGAGTATATTATCTATCATACATTTAATTTAAAAAGTTTATAACCTTGTCATG
ATTAGGCTGGGGTTATGCATTTTTAGGAGGGAGACCCCAGAGGTAAAACGTCAGTTCTTATCACATGTCAAGGTTATGTTCTATTCACG
CGACTGATATAAATCCCGGTTGCATATCCCTATAATACACCTGACCAAAGCCAGGTGTGGTGCCATCTGCCTCTAGTCTCAGCTACTGG
GGAGGCTGAGGCAGGGGGTTGCTTGAGCCCAGGAATTCGAGGCCAGCCTGGGCAACATAGCAAGACCTCATCTCTCCAAAACCACAAAT
TTGGCTGGGCGCAGTGGCTCACGCCTGTAATCCCAGCACTTTGGGAGGCTGAGGTGGGCGGATCACCTGAGGTCGGGAGTTCAAGACCA
GCCTGACCAACATGGAGAAACCCCATCCCTACTAAAAATACAAAATTAGCCGGGCGTGGTGGCACATGCCTGTAATCCCAGCTACTTGG
GAGGCTGAGGCAGGAGAATCGCTTGAACCCGGGAGGCAGAGGTTGCAGTGAGCTGAGATCGCGCCATTGCACTCCAGCCTGGGCAACAA
GACCGAAACTCCGTCTCAGATAAAAAACAAACAAACAAACAAAAAAGGCCAACAAATTTGACCAATACTCCTCAAAACTGGAAAAGCTG
GTGACCACCACCCCTAGTGCTCCAATAAAGGGCTGTGGGAACAGTTGTGCTCAGGGTGGTACTGGTACAGCCTGGCAGAGGACATGATG
CTTGCATAGTGTGGAAAGGCCCTCTGAGATATTGCTGGGCCTTAGGGGAGGAGGTCCAGGCCTAGAGAGGACTGAAGCCCTCCCCAGGG
CATGGTGTGGACCACGCTCAACCGAGGTCGGGTTGGAGACACAGCTGGGAGTCAGATCCCAGGGGCTCTCCTGTGTGATGCTGAATTGC
TGGGAAGGGGATGCCAGCTAGTCTCTTTCTCTGTGCCAACCCTGATTGGTTGGGTGGCTGCTGAGAGCCTTATGGTGCCCTGGCTCAGT
AGGAAGAGTGTGTGTTCATTTAGTGAGTTTGCCAAAGACAAGGGAAGGGCGTTAGACAGTACCTCGCAGATAATGCCGGCCTGGGGATC
CTGGAGGTTTTAGGCTGGCAGACCCTGGCTGAGGCTGTTTTCTGTTCTTTTCCTTAGCCCTTGGGAGACCTTTACCTTTTCAGTGCCTC
TAAGACAGGGTCAGAGCAGTTTGGTGACAAAGTGGAAGTAAACCATGGGTGAAAAACAGCTCTCGGGACCCTCATACCCTTCCACTTAT
TGAGGATTAGGCAGAACTTGCAGCTTGCCCCACCTTTCGAAACAAGGTGTTCCCAGGAGGTGGAAAGACTGGTTTTCAATCAGATAAAG
GTAGGATCCTATGAAAAACTCCTAGGGCCTTGCCGTTCCTATGAAGTGGTAGCTATCATTCACACTCCTAGTTATGCCATCGAGGTAAA
AAGAGGTGGAATAAGAATCAGGGACACTTGAAAATAAGAAAAGAGAAACACTGCGTGCATGTGCATGGGTGCACAGCCTGTACATATCT
GTCTCTGTCCCCCTGCGTGCACGTGCATGGGTGCACAGCCTGTACATATGTGTGTGTGTGCCCCTGCGTGCACGTGCGTGGGTGCACAG
CCTGTACATATGTGTGTGTGTGCCCCTGCGTGCACGTGCGTGGGTGCACAGCCTGTACATATGTGTGTGTGTGCCCCTGCGTGCACGTG
CGTGGGTGCACAGCCTGTACATATGTGTGTGTGTGCTCCTGCGTGCACGTGCGTGGGTGCACAGCCTGTACATATGTGTATTTGTGCTC
CTGTGTGCACGTGCATCGCTGCACAGCCTGTACATATGTGTATGTGTGCTCCTCCTTGCATATGCATGGGTACACAGCCTGTAACTACA
TGTGTGTGAGCCTGTGGGTATGTGAGGGGAGTGAGGGACCCTGAGGGACTAGAAACTTCCTTAAAGGCCATTTGAGAGAGTGCTGCTTT
CTATACCACCCACCCTAGGGCAGTTAGGCAAAGGTGTGCCTCCCTTTAAGAGGACCTAGCGGTCGGTCTGGTTTACAACATAGGTTATC
AGGGAGTAGTACTTTCCCCTCGAAAACCAACCTTTATTTTGGAAAAGATTTCTAATCTCTGTGGCTTGTTTGATACACATTGGAAACCT
GACTGGAAACTCAGGCCACCAGCATCTGTTGGGGGCACATGTTTGCACTTTTGGTTATGATGCCACGGCCCTGGTTCAGGTATCCTAAA
GAAGATAATTAATGATCAAAATGATTGGTGTAGATTTCATACCACAGAGCACTGTGGTTTGTATAGTAAGACACAGTGATGGTGTCGAC
CCATGCACACCCAGCTTTCCACATCTTAACAATACCGACTTTGCCTGACATAATTGGTGTCTGCACATGGGATTAAACAAGTAAGATTG
ATGTGAATTTTATTCTTTCATTTTTTATGTAGACTCACTGATTGGGTAACTGTGGAAATAATGTCACAGTGCCTTTTTAAGTTAAGAAC
ATGTAGTTGTGATCAGGAGCGGTGGCTCACGCCTGTAATCCCAGCACTTTCGGAGGCTCAAGTGCCTCGATCACCTCACCTCACCAGTT
CAAGACCAGCCTGGCCAACATGGCAAAACCCCGCCTCTACTAAAAATACAAAAAGTAGGTGGGCGCCTGTAATTCCAGCTACTGGGGAG
GCTGAGGCAGGAGAATCACTTCAACCCAGCAGCTGGAGATTGCAATCACCCAAGATCGTGCCATAGCACTCCACTTTGGGTCATAGACC
GAGACTCTGTCTCAAAAAAAAAAATTAAAAAAAAATAAAAAAAGATAAAATCTAGTTATCAAAACATTTCTAAGCATTTTTAAGCATTT
TATATTAAAACAGGAATTCAGAACAAATCAAAACGAAACAATAAAACTGGGGATGAAGGAAACTCAATTTTAAATATAAACTTTGGCAG
GTGGGTGGTCAATCCGGGTGTTCTCAGTCATTTGCTGGCTTGTTTGTCACATGGCCTGCTCTATAAGTGTCACCCTAATTGATCTATTC
GTTTGGAGTGGGTGCTCTTCTGAGGTTTTTGTTTCCCCCTTTCTTTGGGCTGCACTGTCTGTCCATGGTAAGAACAAGGGGCCATGCAA
TTAGAGAATCTTGAGTTTCTAATCCAAATTCAGTTCCAACCAGTGTCACCTGGTTCTTTAACTCCTTTGAGCCACCCCCGCCCATGGCC
CTCATCTGTACCCTGAGGATAATAGTGTGGGCTTTGCAGGCTTTTCGTCACCAAGTGACATGATGTAGCAAAACACCCAGCCCAGAGCC
TAGCACCAATTGGTCTCTAATCCATGCTGCACGGACACAGCCATTCTCTGGATCTCGCCTCTTCTGCCTCCACTGTGAGCTCAGAGACT
CACTCACTCCACGACTAACCTCTCCTTGGCAAGCAGCGGGAGTCATTTCATCCCAGCCTTTCAGGAGGGTGAATCTCCACCTCGGGTCC
AGAGTCTCAGAGATGAGACGTGAGCCAGGCGCTGATTCATCATGATGCAGGCTGTGCAGACTCTACCCATGGTTTCTCCATGCAGGAGT
CAGGTTGGGATAAGGGGTCTTTCTCGGGCCTCTGTGCTCTCTCGCCCCTGCTGCTCCGGACTCGTTCATTGGACAAACCTCTCACATTC
TCTAGACCGGTTGCCACGCCATGCTCACAGTCTCTGTTCTTGCCTTCCTAGGTGGGAAGTGAGTGATGACCCTGAAGTGAGGACTCATC
TCTAGATCTCCAAGGGCTGCACCTCAGCCACCACTTTACAAGCGTGATCTCCAGCCAAACTCCCCTCTTGGCTGACCATAGGTGACTCT
GGGTAGCCCATACCCAGGCTCAGCAGCAGTTGGGGAGCTGCCTCGATTTCTGGTTACAGAATTCCTGGAACTGAGTCACTGCAGTAATT
CCTGTGATGAATTGTGTTTACTTTGTGTGGGATTCCAAACTGTAGCACCAGTGACTACAGCTGGAAGACAGCATCATCAGCAGCTTCCA
AGCCAGAGCCTGCCGTCAGAAAGCTCCATTGCCCTAATGCTCAAGCCTGTGCGAGCCTGTTGGAGAGACACTTGGATGTTTAGCGAGCT
GGTGACTCTCCTTGTCATGAGTAAGCTTAGGACCTTGGGCAAGTCATCCAAACTCTTCTGCGCAAGTCATTCTCCTGCTTGGATGCCTT
GAGGCAGAGAGGCAGTCAGGTGAAGTGGTCAGTGCGTCGACTCTGCCTCTACCCTCCTCGCCTTTGAATCCACCTGTGTGATGTTGTAT
GATATTGACCTTTCTGGCTCTCAGCATCCTCTTGTGTGAAATAGGAGATTTTAACAGTATCTATTTCGTAGGGTTGGTGTTTGAATGAG
TTAACATATGTAAAGTGAATGGTACAGTGCCTGGCTTCCTGGCAAGATTGCTATCAGGATTAAGGCAGCTTAAGCCCTTGGCACACACT
AAGACCTCAATAAATGTCAGCTGATGTTATTGGTCCTTTATTACTATTCAAGAAGCCTGCCCAGCCCTCCTCCCTCTCCATCCACACAG
CAGCCTGGTACCCGCTGTTCTCTAGGTTCTGGACACACGTTATGACATGTTCTGATGATCTGGCTTAGACAGTGGGGCCCTCGAGGTAG
GCCCAGAGGACTTGGTCCTCACTGCCTCTGTGGCGCCTTGCACTGGGTCCAGCTGACGTGGAGAGAGACTCAGGAAACAGTGGCTGAGT
GTGACTTTGGCTGGCATAGTGGTTGCTGAGAGAACAGACAAGGTTCTCTCTCACGACATACAGATTTCAGATCAGGGAAAGTCCCAGCT
GGCATAAGTTTATCGAGCATCTCCCATGGACAAGATCAGCTGTGGGTGGAGCCTTGAAGTACATGGTAGAAGGACACCGAGTCTTCCCA
CGCCACCGCTTCAAGTGAGGAGACAAGATATAGCCTCCCAGAGAATTCCTATAATGCAATCGTGAAAGAACCATACCCAGCAGGAGGCC
GGGGAAAGTGACTCCTGCAACTCTAGGAAGGCTTCCTGGAAGAGGTGGAACGTGAGCAGCATAGGATTTTGACAGAAGAAATGGAATGG
GCTGAGGGAGATTCTGCTGCTGCAGCTTCAGGTTGACCTAAGGGCTGGCAGCAGTGGAGGCCCCCCACGAGTGAGTTTGAGGGGCCTCT
TTAGCTCAGTCCAGTTGAGGCAGCAGAGCCTTTCCATAGGGGTGTGGTGTGACCTGAATGTTGGGCACGTGGTCGTAACTGAGCTTTAA
AAGTGAATGAGAGGAGCCATGCCTGATGGCTCGAGCCTGTAATCCCAGCACTTTCGGACATCAAAGCTGGGGGATCACCTGAGGTCAGG
ACTTCGAGACCAACCTGGGCAACATGGTGAAACCCTGTCTGTACTAAAAATACAAAAATCAGTTGGGTGTGGTGGTGGGTGCCTGTAAT
CCCAGCTACTCAGGAGGCTGAGGCAGGAGAATCGCTCCAACCTGGGAGGCAGAGACTGTAATGAGCCAAGATTGTGCTGCTCTACTCTA
GCCTGTCTCAAAACAAAAAACAAGAAACAAAAACAAAACAAAACAAAAAAACACTGTCTCAAAAAAAAAAAGTGAATAAAGAGAGAAGA
AAGGCGGGGCACGATGCTTTTTAAGTCTACGAGCATCTTATAGCATTGTTTACATCCCAGCTTTTCACTGACCCTTCCTTTACCCCCGC
CCCCATCCCGTGGCCCTTGCTCTGTCCTGCCCTTTCTGTACGGCGTTTTCTCTTCCCGGCTCCTCTGCCCCCAGCCCCACGTGCCAACT
TGTTTCAGCCTGGCAACGAGGCCTGCTGGTATCCCAGCCATTTCCTGCCCCAAGGCCGCCCACCCTCCTCCGATCTAGCATGGCCTTGC
CCTCCGTAGGCAGGCCTCACCCCTGCGGCAGCCGGGAAGCAGGGCTCCTGTTTTGCCTGTTTACCCTGGCGCAGGGACAGGTAGATCAT
GTAGCACTGCTGGGATGCCAGCTGCCTAAGTCTCCGGCAGTGGGCACAGGCGGACGCCACGCCCCATTCCTCTGGGCTGACTCGAGCCC
GGCCTGCCTGGCACAAGACCCCCTGCTCCTGGAGGCAGCTCAGGGGCTTGCTCATGGCTGCCCTGGCTCTCCGCTGGGCCGCTGAGTCA
TCGCTCAGCAGTGAGGCCTCTGAGTCACAGTGCAGCCCTGGACTGGACAGTGAGTGCCTTGTGGGACCCTCCTGGGCTTTCCTCTCCCT
CAGAGCATTTAAAGGGGTCCCAAATTGTCATGTGACCCTCTCTGATGCCAGACAGACACTCTAGGTCTTTCGCGCCCTGGTCATTTTAC
AGAACTGTGGCTGGATGCCAGGCATTCAGAGATACATTCCTCAATCTCGGTCCGCCTTACAGGATCCAGCATCGAAGAGTTCAGTTCAA
GTCAGCAGCCGAGTTACAGGAAAGCAGCCAGGGCAATGATTCAGCAAGTCCAGGCAGCAGTGAACCAGGCAAGATTTCGGGAGCAGAGG
CCTGGGTCGCGTGTCCTGGCAGCGCCACAGGAAGAGCATACACATCTCCTGCGTCTTCGTATTTTGTGAGTGTCTTGAAATGACCTTGA
GAATCTACGCAATACCATGAAGAAGCACGAGCAGGAATACCATTTATCCATTGGCACAGGGTAGCATGCTCAGTGTGCACTGAGGTTGT
GAATTCTTGTCCTGACCCAAAGATGCTGATGCTGTCCTTATACCCATTTCACGCTTCAACAAACTGAAGCTGTAGTAGGAGAAGATACT
GACTCAGAGCCACAGAGTTAGAAAGCAGCATGTCTGGGATTTGACCCCAGACCTGTCTGACTCAGTTTACTATTTTAAACTACCCGATT
CAAGTTAAGTTTCTCTCCTCTCTCTCTGTCTCTCTTTCTCCCACGTGGCTGCTGGAATCTTCCCTAAGGACCATACCTGTCTGCAGTGC
AGGAAGCCAACTATTAAGGGGAAACAAAACTGTTCTTAGGAGTCCTGCTGTCACTGTGGATTGAGCCGGTGAAGCCCTGAGGGATTTCA
TCGCTGAGGTGGATGGAGAAGCAGCTGGGGGCCTTGGCTGTAAGGACCCCTAAGGGTTAGTGTCTGGAGCTTGGGGGATGGGAATCGGT
CCCGGTGTGCGGAAATTGGATGGCTCGCTGACCCCAAGGGGAAACCCTTGTTGACGCTGTAAAGTGTCTTCTGGCCTTAGAGAAACCCT
CTAGGGTTCAGGTTCCAGCTTCACCCTCCTTTAGTCAGGTGGCTTTGGGGGGGACATTTCTGCTGGCCCTGGCAGACACTGGACTAGAG
CAGTGTTTCTAAAGTGTAGCCCCTTCTCTCTCCCGGGAAGGGAGACAAGTGCGTGAGGGGATTGGAATCTGCATTCATAACACACTCCC
CAGGAGAGACTGGAGAGCACCTTTCCCCCTGAACTCCCGTCATCTCAGGCCTCAGCAGACTGAGCGGGAACCCCCACCAGCGAGGTTGC
TCAGGACAGCGTTTCTCTTGGGCCTGTGTTTGGAACAAGTATCCTTTGAACTAATCACTAACCTGTGAAAATGGATACACCTCGTGTGA
AAATGAAAATGTCTGGCTTCCCTTCTGAGGAGCGGAGGCATGAAAGGTGTGGCCGGTAGCGGTGGGGCCCCCACACCTGTGCAGCCGCC
CTGGCCAGAGCCCACAAGCAGCGTCCCCTGGTCATGCCCCACTCCCTTCTCCATAATCTCGCCCCTCCCTGACCAGGCCACCTGACCTT
GCCATTTATATTCAAGTTCATTCCGTTGTTATTCTTTTAGAAAAGAGAGATGAGAAATATTTCTTGTGCCTACATCTTTATCAAAAGTA
GAGCAAGAAACTTGAGGACTCTGTTTCTAGAAAAATGAGAGTCGGCATCCCTCTTCTCTGAAAGGAAGAATTTCCATACCTGCCCTCTA
CCCAATGTCTAGACGAAAGGATGAGTGTCCTTTTGGTGTGTGGAGAGCTTTGATGGGGCATGTGATGTGTAGGGCTTAGGGGGTATCCT
GCCGTGGCTGGGAAAGGTGGTTGGGTGTCCTGGGAAGGCTTTTATACACACTCCGTGGCAGGCCTAGGACTCTAAGTGCTTCCAGCCAG
CTGTACATACGTATATATTTTTTTTTCTTTTTTTTTTTTGAGACGGAGTCTTCCTCTCGAGTCCAATGCCATGAACTTGGCTCACTGTA
ACCTCCCCCTCCCACGTTCAAGTGATTCTCCTGTCTCAGCCTCCCAAGTAGCTGGGATTACAGGCACCTGCCACCACGCCCACCTAATT
TTGTATTTTTGATAGAGACGGGGTTTCTCCATGTTGGTCAGGCTGGTCTCGAACTCCCGACCTCAGGTGATCCTCCTGCCTCGGCCTTC
CAAAGTGCTGGGATTACAGGCGTGAGCTACCACGCCCAGCCCCAGCCATCTATATTAACTCAATGCAATTCTTGTTGAGGACTAAAATG
GAGTTTTTGTTCAACACTAAATAAGTAAATGACAGTGGAGTATTGTCATTATCCCCATTCAATAGATAAGGAAATTAAGGTTTAGACAC
TCACAGTAACTGGTCCTGGCTACACAGCCAGTCAGGAGCAAGGCTAACATTTAGGCCTAGAGTGCTCTCCTAACCGCTATGCCTGGGGT
GACCACCCAAACCTTCCATCCTTGTGTCATCCCATTTGATATATTAGTTACTCAACCTGAAGCTAAAATAATCCGAGGCCTGGGCGAGG
TGGCTCATGCCTGTAATTCCAGCACTTTGGGAGGCCGAGGCAGGTGCATTGCTTGAGGTCAGAAGTTCGAGACCAGCCTGGCCAACATG
GCGAAACCCTGTCTCTGCTAAAAATCCAAAAATTAGCTGAGCGTCGTGGCAGGCACCTGTAATCCCAGCTACTCGAGATGCTGAGCCAG
GAGAATTGCTTGAACATGGGAGGAAGAAGTTGCAGTGAGCCAACATTGTGCCACTGTACTCCACCCTGGGATACGGACCACGACTCTGC
CTAAAAAATAATAATAATAGTAATATAATAATAATCCGAGAGTGCTTACCATCTGCCTACATTGTTCTGCGATACTGAGATGAATAAGA
CCTCTTTTGGGCTCTTGATCACTTGGTTATAAGAAGACGCAGCTCGGTTCCCCCAGGGACTCTCAACAGTTAGCGACAGGCTGGGGCAT
CGTGGCTCCTGCCTGTAATCCCAGCACTTTTAGAGGCTGAGGCAGGAGGATGGAGTATGTCCAGGAGTTTGAGACTAGCCTGCACACCA
TAGTGAGGTCCCATCTCTACAAAAAATAAAAAATTAGCTGGGCATGGTGGTGTGTGCCTATACTCCCAGCTACTCAGTAGGTCAAAGAC
GAGGAGATGGGAGAATTGGTTCACCTCTCCACTTGGAACCTCCAGTCTGCAGTCATCACACCACTGCACTCAGTCTACGCCACAGAGTG
AGACTGCCTCAAAAAACAAAACAAAACAAAGCAAAAACAATTACGTACACAAAGGCAGGAGCATCCACATTTTCTGACATTGTTTATTG
CTTTTTTACCCTCATGTTCTCCCCTCTCCTCGCATTGCCTGGCACTTGGGAGAACTTTCATAAATATTGGTCGAATGAATCAATGGATA
TTAAATCATTTGTGTTTTCTTTTGTGAGTTGAAACCGTCTTCAAATGAGTCTTCAAAGTGCTCTGATTCGTATGGTGGAGTGTGATGAA
GTTAAACTCTGGGTTGAGAAGAGACGAAATGTGAGTTAAGGAAATAAAGAGAATGGAGTTGAATGTTCAGAATCTTTGGAGTGCTCTCA
GGGATCCATCATTCTCAATTCAGTTATCATCTTTGCTGTGAGAAATCCCCAAAGATTCAAACTTCTCACCACAAAGGTGGTCTTACCCT
GGGTGTGGTGGCTCATGCCTATAATCACGGCACTTTGGGAGGCCAAGGCGGCTGGATCATTTGAGGTCAGGAGTTTAAGACCAGCCGGT
CCAACATGGTGAAATACAACATGGTGTATTTTGTGTCTCTATTGGAAATACAAAAATTAGCCGGGCATGGTGGCGGGTGCCTGTAATCC
CACCTACCACGGACCCTCAGCCAGGAGAATCACTTCAACCTGGGACGCGCAGCTTGCACTGAGCGCATATTGTGCCATTGCACTCCAGC
CTCGGTGACAGAGTGAGACTCTGTCTCAAAATAAATAAATAAATAAATAAATAAATAAATAAATAAATAAATAAAAGATCGTCTTCAGG
TTTCCGTTTGGGCATAGGTTACCTTCTGCAGAATGCATGTCAGTGATATGGAGACAAGTTTTAACATGGGCACTTTTTCAAAACAGACA
TCTTAAATATTCGCCAAAACCCCAGATCTCAAAGTGGACTTTCTATTTTTGATGGAAAATTTCAAGCCTGTTTTCTGTAAAGCAATGAT
TATTGTACAAGGATTTCTGGAACTGCAACTGGCCCATAGATGGTGGCCATTATAGTAGTTTCCTATGAAATCTACTAATGTACAGCAAG
CACTTCTGTGACTTTCACTCACCACTTGGTTTCTTTTTTTTTCTTTTTCTCTTGTGGACTACACTTGAACAATCCTTTTTTTTTTTTTT
TTTTTTTTTTTTTTTTGAGACAGGGTCTTGCTTAGGCCGGAGAGCAGTGGTGCTATCATAGCTCATTGTAACCTTGAGCTTCTGGGTTC
AAATGATTCTCCTGCCTCAGCCTCCTGAATACCTGGGACTTTCGGTGTGTGCCACCACGCTTGACTAATTTGTTTACTTTTTGTAGAGA
CAGGGTCTTGCTATGTTGCCCAGGCTGGTCTTCAACTCCAGAGCTCGAGTGATTCTCCCGTCTTAGCTTCCCAAAGTGCTGGGATTACC
AGCATAAGCCACCACACCTGGCCTGAAGGCTCGTTTGTAATCGCACTCTTAAGTGAGATAAATTGACTATTTTCTTCCTCACAACCTTC
TTATGCTTGTCTGTAGTTGGCTGTCTCATTTTGGACAATTTTGATTGTCGGCTTGGCTCTTCAACTGCCTGTGTTGTTGTATTCTTACA
ACACACAGAACAACCAGGTCTGGGCTTCTTATCTTGCGAGGCAGGTGAAGAAAGACTAGGAACAATGTATTTGGGTGCCCACCTCATAC
CGCCCATGACCTTCTATTGAGGAACAGCAGGTAGATGAAGCAGCTTTCTCATATTACGCCGTGGCTGACTCCAAACTTGAACTCTGGTC
TCTGTTTTTGGAGCTTGCTTCCTTCCTACGACCCTAGACTGCTCCTCCTAAATAATTAAGTGGCAAACATGGCACAGACAGTAACCTCA
CATAAGAAGCTATGAGGTTAGGACAGAAATATCCACTGCTGGCCTGCGAACTATTCACACAGGTCTGACTCCCCGCCAGACTCCTGGCT
GCCCTGGTCCTGTGGAATCCCGTCATGGCTTCTGTCTAAACGGAGCAGGCTCAGGGCCATGCTGAACTTGGAGAAATCTGTGCAGGAGT
CAGATTTGGGCTTTAGAGTCACCTGGGGTTGAATCTCGGGCCTGCTATTTATTGAACCTGTCCTAGCCTCAATCGCCTTATCTGTACCA
TGGCCATCATACCATCTACGTCGAACGTAGTTGCAGCAGATTCTCATGCACGCAGGCCCTTCCCCGTCATTTCTTTCTTTGTCTCTACA
AGCAGAGAGGCTGAGTCATACAGATCCTGAGGGGAGATCACTTTCTCTTCCCTCCTTTCGTGTGTCATCTCCACCTGGGTGTGGTGAGC
CCCAGGAATAATGCTGTGACATCAAGCCCCGTGAACACCTCCTTTCACACGGACCCCGTGACTGGCTGCGGCTGCCAGCGGTGATGAGA
ACTGATGACACTCCAGGGCTACCAGGAACGGTGTGTGTGCCTCTGGGCAGGGCCACTTCCTAGAACCATGGCGCTGCTCTCTGAAGACA
ACCGGCTCCCAGTGAGCTCCAGGAGACGAGGAGCAGGTCCTGGTGGCGGAGGTGCTGGCGCAGGAGGCCGGCTCCTGGCAGAATCGGCT
TCCAGCTCTGGGCACTGGGGTGCGGTGTCGGACCAGCAACAGGCTGCTCCTCTGCCAGGAACCGCCAGAACCAAAGTCTTCTCGAAGCC
ATCTTCAGAAGGCAAGCACAGGGCTGAGCACTCCGGAGAGGAGCCTTTGGGGGAAAACTTTTTCTCGGAGCTTCGGAGCTCAGGGAGTA
CTATGGGGGAGATTCACTAGGTTGATGCTTTGCTGATAGGCAGCCCTGTAGAGAAGACAAGGCAGCTGGGCATGGTGGCTCAGGCCTGT
AATCCCACCACTTCGGGAGGCCAAGGTGGCCGGATCACAAGGTCAGGAGTTCAAGACTAGCCTGGTCAACATGGTGAAACCCCGTCTCT
ACTAAAAATACAAAAAAATTAGCTGGGCGTGGTGGCACGCGCCTGTAATCCCAGCTACTCGGAAGGCTGAGGCAGAAGAATTGCTTGAA
CCCAGGAGGTGGAGGTTGTACTAAGTCGAGATCACGCCACTGCACTCCAGCCTGGGTGACAGAGCAAGACCCAATCTCAAAAAAAAAAA
AAAAAAAAAGGCAGAAGACAAGGCGACAACTGAGCTTTCTGTCTCCTGTGCAGTTTACCACTTGGTCAGGTGGCCATTTCTCTCCCTCC
TACTCGGTGACCTTCTCAAGGGCAAGGGGCTCCTTGAAGAGTGTCTTTCACATTGAAACATGCACAGAATGGTGGCTTATGACCCATGG
TAGACGCGTGAACTTGGGATGCCACTGTGGACGTGTCCCCAGCCTCCCTCCTCATTGGTGTACTGATATCGTGTTGCCTTGGACCTATT
ACCTCACCTCTTCCTGGTGATTTCACCTCAATATCCCCTTTCTCCTATGCTTCTGTTTTTCCTTCTACCCTTCCATGTTTTTTTCTCCC
AACCATTCTTCTGTATTTTATTCCACCCAGCCACCCTTTCATCTAACCAGCCTTCCACCTAGCTAGGCTTCCAACTAACTATCCTTTTT
TATTGAGACACAGTTTCAACTGGCACAGTTGGGCCACTTGTTTCTCATCGGAGCCACTTCTCAAACAGGTCGATCACTAGGACTTTGGA
CAAAAGCATGCGTGCAGGCTGGGCGCCGTGCCTCACACCTGTAATCCCAGCACTTTGCGAGGCCAAGGCGGGTGGATCACCTGAGGTCA
GGAGTTTGAGACCAGCCTGGCCAACATGGCAAAACCCTGCCTCTACTAAAAATTGATCAAATATAGATCACCCGTAATTATCACCCATC
ATTCTGACACTTAGCAATAGTCACCCTTAACATCTTGTTGTCTTGCTTTCAGATGGTTTTGTTTTTGACATGTTTTTTTTTTAACTTAT
ATTATGACCATCTCCTCTATGTCTTTTAAAGTTTTAAAAACTTGTTTTGAATATCTTTTCCATATTTCATTATGCACCTGTACAGGAAA
GGTACTCCATAACTACGTTTTTCTTCTCTGTAAAATCGGGAAAATAAGAACCCCTTCTACATGGGGTTCTTCCGAGGATGAGGTGAAGA
AAGTTGTCAAAGCCCCTCGCTAGTACCAGGCACAGAGCAGGTACTGGGCGCATGTTGCCTGCTACTGGTATTATTGTCATTGTCATTGC
TAATCATCAGCCAAGTCAGTCCTGCGGGGCTTTCAGATTTCCTCTGCTTTTACCTGTTGCAAGGTATAAACCTTTTCATGTGTCTCTGA
TTGTTTTCTTGGCAGAGCCACCCACACCCCAGGAATTTAATAGGGTCTTGATGCATTCCCAGCTTGCTGTTGACGAGTATTTAACCAGT
CACCACTCCACAGGCAATATTAGGGGTTGCTGAGGCAGCCTCCAATAGTGTGAGCATTCTAATTACTGCAGCAAACTCTTTTAAAAAAA
TTTTAACTTTTCTGGCTGGGCGCGTTGGCTCACGCCTGTAATCCCAGCACTTTGGGAGGTCGAGGTGGGTGGATCACTTGAGGTCAGGA
GTTCGAGACCAGCCTGGCCAACATGGTGAAACCCCGTCTCTACTAAAAATACAAAAATTAGCCGGGCATAGTGGCACATGCCTGCAATC
CCAGCTACTTGGGAGTCTGAGGGAGTAGAATCGCTTGAGCCCGGGAGGCCTAGGTTGCAGTGAGCCAAGATCACGCCACTGCACCCCAG
CCTGGGTGACAGAGTGAGACTCCATCTCAAAAGCAAAACAAAACAAACAAACAAAAAACAAACCACCCTGCAGTACTACTCAGCAACAA
GAAGGAATGAACTACTGATAACACTCAGTGACTCGGAGGAATCTCTAAAGGATCATGCTGAGGGAAAAACCCAGTCCCACAAGTTTACC
TGCTCTATGATTCCAGTTACGTATAACATCGTTGAAATGAGAAAATTTCAGAATTGGAGGGCAGATTAGTGGGAGAAACAGGCAGAGTC
TACAAGGGATCCCTCCGTTATTTCTTACAACTGCATGTCAATCTATAATGATCTCAAGAAATAAAAATTCAATTTGAGAAACCTGCAGT
TCCAAGTCTGCATTGCAGCTGGAGGTAGTCANNNNNNNNNNNNNNNNNNNNGGCGCGTTGGCTCACGCCTGTAATCCCAGCACTTTGGG
AGGTCGAGGTGGGTGGATCACTTGAGGTCAGGAGTTCGAGACCAGCCTGGCCAACATGGTGAAACCCCATCTTTATTAAATATACAAAG
ATTAGCCAGGCGTCGTGCCAGCTGCCTATAATCCCAGCTATTCACCAGCCTCAGACAGGAGAATTGCTTGAACCCAGGAGGCAGAGGTT
GCAGTAAGCTGAGATCATACCACTGCACTCCAGCCTGAAACACAGAGTGAGACTCCATCTCAAAAAAAAAAAATTAACTTTTAATTTTT
ATTTTTTAGAGACACGGTCTCGCTTTTTCACCCAGGCTGGATTGCAATCATACGTTCATAGCTCAGTTCATAGTTCATAGCTTAGACCT
CCCAAGCTCAAGAGATTCTTCCACCTCACCCTCCCACAGCTGCACACCACTATGCCCAGCTAATTTTAAAATATTTTTTCTACACATCA
GGTCTCACTATGTTACCAGGCTGGTCTCAAACTCCTGGCCTCGGGCAATCCTGGACTCCCCAGATGCTGGAATTATGGGTTGAGCCATA
CCCTGCACCAAACTCTTAAAACATGAGGCGAACTATCTTGTTATAAAGTTGCTTTTCTTTGACTACTGGTGTGGTTGGCCAGCTTTTCA
TGTTTATGGCTTATTTGTGTTTGTTAACTGGTCATTTCCTTTGGGGTGTTAGCAATTTTTTTCTCATTGATTTGTAAGATCTGTCTTGA
TTAACTTGTGAATTTTGCACATGGTGGAACCGGGGTGCCCCACAGTAACTCCCCTTGGTTAGCCATCCTGTCCCTGCATTCCACACCTC
CATTTCTATGGGGGACATTTCTTTACAAATCTGCATCTGATTGTGATACCAAACCATGCTTTGCAAGTCAATACAAGGATCTACCCCAT
TAATCTGCCCAGTTCCTAGTTTTATTTATTTATTTATTTATTTATTTATTTATTTATTTATTTATTTATTTTTCCCACACCCTCTCTTG
CTCTGTCACCCAGGCTGGAGTGCAGTGGCATGATCTCCGCTCACTCCAACCTCCCCCTCTTGGGTTCAACCAATTATCCTGCTTCAGCC
TCCCGAGTTGCTGGGATTACAGGTGTCCGCCACTGCGCCAGCTAATTTTTTGCATTTTTAGTAGAGATGGGGTTTCACTATGTTGGCCA
GGCTGGTCTCGAACTCCTCATCTCATGATCTTTCTGCCTCAGCCTCCCAAAATGCTGGGATGACAGGCATGAGCCACCGTGCCTGGCCA
GTTGCCAGTTTTTAATGTTCTCAGGTGAGTGGCATTTGCCAAGGAACTTCTCTCTTCCCTTCTGTCTTCATAAACAAAAAATCCTACTA
ACTTCAGGTGTCAGTTTAAAAGTCATCTCTTCTGGGAGCCCCACCTTAAACCCCTAAATCAGCTGTTGGCGTCGCGGCTGGACTTCTCC
TTTGTTGCACCAATTGCAATTTCTTACTTAGGTGTGTGCTTGTGGGAACATTTTTCTAATATCACCTTCTCTGCTAAGGACAGCGATCA
TGTCTCTTTTATCATTTGGTATGTGTTGCTGCACCACTTACCACAGTCCTTAGCTTGGAACCCAAACCCCACAGCGGTACAATCACTTG
CTTGAGGCCCCTCAGCTTGCACCTGGCACATCTGGGACCCGGTGTCTGGGCCCACCTTCAACCTGCATCCCTCAGGACCCTGTGTACCT
CTGGCCAGGGCTTTGCTTTGCTCCCTGGATGAGAGTGGTAAGAGTTGCTGAGGTGCCAGGCCTTTGCCCCCATCCCCGCTGGCATCACT
GATTCCAGGTTATTTTGGCTCAGCGCGGATGGAGCGAGATGTAGGGGAAAGGTTGGGACCGGCTTCCAGGTGATTGGCACATCCCTGCC
CTAAACCCTCCATGGCTTTCCTGGGCCTGCCATCCAAGGGGAAAGAACCCTCTTTCCTCACAAGCTGAAGCTTGAAACCAATCAAGCTC
CAGGCCAGAGAAGCTGCAGGTGGGGTGTGCTTTGCAGTCGGCGTGGGGCAGGAAAGAAAGGTTACGTGGGCTGAACGGGTCAGGTCAAT
TCAGTGTGGGTTGGAATGCTGGGGTCCTGGGTGGGCAGGGATGAAGGACACCTGGGGAGGGGAGAGAGCCTGCCGCCTGGAACTGGGGC
CCTGCCAGTCCCCCGCTGCATGAGTCTGGGCATGCTTCCAATCTCTGGGAGCCTCAGCCTCCTGTCTCTAAAATGGAGCTAATGAGGCC
TGCCGTCACGCTTTGTGGGCAAGATTCAGTAACTGGATGCCTCCTCCTGGCCCTTGGCAACCATTCAATAAATGTTCATTTCTCTCCCA
CCCCTCGCTCCTTCTCTTATTCTTTTCCTGTTGCTTTTCACACGGACATGTGTTGAATGTGGCAGGATATTTCTTTCTTTTTTTTTTGA
GACAGAGTCTTGCTCTGTCACCCACGCTGGAGTGCAGTGGCGCGATCTCGGCTCACTCCCAGCTCCGCCTCCCCCCTTCACCCCATTCT
CCTGCCTCAGCCCCCTGAGTAGCTGGGACTACAGGCGCCCACCACCACGCCCGGCTATTTTTTTTGTATTTTTAGTACATATGGGGTTT
CACCATGTTAGCCAGGATGGTCTCGATCTCCTGACCTCGTGATCCGCCCGCCTCAGCCTCCCAAAGTGCTGGGATTCCAGGCATGAGTC
ACTGCGCCCAGCCACGATATTTCTAACACAGAGAAATTCTCAGTGGAGGCCGCGGGAACTAGAGATAGAGGTCCTCTTGCCTACCAAAG
CACCAGATGGAGCTGACAGAGGCCATCTTGTCCTAGAGGGACCTGGCTAAGAGGATCTAAGACCAGAACTACTCTGAGTGCTTGGGATT
CATATGGGGCTGTACACGCCAGGCCTGCTTGAACACCAGTGTTCCAATCCCACATCTCATTCTTAGAGCTCTGTGGCCCTGGGCGAGTC
TCTTCCCACTATTTCTTCATCCTGAAGTGGGCACCACATTACTGCACCTCAATTTGGACGAGGATTCTCAGGATGGGGAGGTGGTTGAA
ATCATCCAGGGGTCACTGGAATCAGGATGGTCCTGGCTTTTTCAGAAGCACAAGGTCGTAGGTGCCAATCTGGGGGCTTTGCAGATATT
GAACCGCAGCCTGAGGATCGGGATAAACCTGGGTGTGGCAGACAGAATTATGCTTCCCCCAAAGAGGTTCACATCCTGATCTCTGGAAC
ATGGGGATGTCATATGCCATACAGAGAAGGGAAATTAAAGTCACAGGTGGAATTAAAGCTGACCTTAAAAGAGGGAGATCCTTTTAGAT
TATCCAGAGCGCCCAGTCTCATCACAGAGTCCTTACAACTGCGCCACAGACGCAGAAGAAGGGTTCAGAAACATGTGAGGTGACAAGTA
ACATTCCTGCCTTTGAGGATGGAGGAAGGAGCCATGAGACAAGGAATAACCGTCCCTAGAAGTTGGAAAAGGCAAGGAAATAGGAACTC
CCCCAGAGCCTCCAGGAAGGAACACAGTCCTGCTAACACTCAGTGAGACCCACTTTGAACTTTGTATCTTCACAACTCTATCATAATAA
ATGTGGGTGGATTTAAGCCTCTGAGTTTGTACTAGTTTCTTCCGGCAGCCCTAGGAAGTGAATGCAATGGGCGCAGCATTTGATGGCCA
GCCTGGCATCCAGCCTGTGGGGAGTGGGAGCTGAGGGCAGTCCAAGGGAAATGTCCTTTGAGTGTGGCACCGGCAGGCAAACCCTTGTT
TGGGTGTGATCATCAGTGAGTTGGGCCACTGAAGCTGCCTGGTGACCTGGCAGGGATGGCTTAAGTACCCACCGTTTCCAGATCAGCGA
GATAAAGGCGCTTGCATAAATTCACATGCCCATGGCTTCTCCCTCTGACAGCGCAGCCCTGCCTCCGGCTGCCTGGCCAGGATGGCGTG
TTGGTGCATTAGGGACCCCTTCCTCTCCTGCCCTGCTGTTTAAATTGTTGGTCTGTTGGCGTTCTCAGGAGCCCTTTACCAGTACCCTT
AGGCCCCAGACGACCTACTCCTCAAGCTGATCATATCTGCTGCCTGTCTCCCTGCTCTGGGCCCAGCACCCTCATGGTTTTCCATAACA
CGTATCCCAACTCCTCCTGATGGGACCCTCGTGAGCTGTCTGTGTAGGGAGCATGGCTGCCCCTGGGTCTGCTTTCCTGCCCCTGACCC
ACAGGAATAGGATGTGGCAGAGTGGGGGCCAGATGACAGAGATGCTGGTGCTCTCTCAGCTGAGGCCCACTGCTGACCTGCTGGTGGCT
TGTCCTGCTCTTGGATCATAGGCACTGGGACATCCTGCCTTCCTTCACTGTCCCCTCCCACTCCCTCTCCTTCAGCATCAGAGGAATGA
AGGAGGCCTGGAGGAGTAGCCCCTATCTAATAGAGACAGGAGTGGGCTGGGTGAGCATGGGGTGGTCGCAGGGGGAAGGGGATAGGAGA
TGGAGAGAGGAACCTGCTATCTGGTGGGTTCCAGGATGGTGACAGACAGTGGCAGAAGCAGTGCAGAGACCTTAGTTTGTGCTGCACGT
TCAGTTAATCACTCGTCAACGCAGACATCACTGCAGGAGACCAGACTGTTATGTATTGTCTGTGCATTCATATTAGTACACAGGGAAGA
TCTTGGGCTCCGAGACCTAGGCAGATCTTGCCTCCCATCTGCCGACCCTCCGCAGATGCTACACATTATGCCAGGTAAACACATTATCA
AGGAGCCTTGGGCCGTGTTAGAGGAAGGCTCCTTGTGCCTGTGACCAGTTTGCACTAATCCTATGTGTGTTTGTGTGTAAGGCTTGCAT
TCACTGATTCACTGTAGAGGATGACTTGAAACCCAAGTCATCAGCTGGGCAGTGCATGGGCTGCTCGCCTGAATGGTGAAGCTCTACCA
AGTGGTTTCCACCTAGGGCCTGGCTTAGCTGGCCTCAGATGCCCTTCTCAGTTCTGCTCAAGACCTGCCTGCGCCCTTCCTCGGGCTTG
CTTGCCCAGAATCCTAGCAATGCCCACGGATACTGCCTGGGCATGTGTTTGGCCTCAGCCAAGGTCAGCCTGTCAGGAAGGAAGAAGGG
GGAGGGCCACACTTGCCTGGGAGGGTGGAGGAGGAGGGGTCCAAGCCGCCACAGCCTGGCAGTCCTTTTTTGTGCAAAGCAGACCCATC
AGCTCCTCTGCCTGGAACACCCCCATTTACTCCTCCGCAGTTAGAGCCGTGCGACGTGAGGAATTTTGTGAGGGGAATTATCTCTGATC
TTCATGTGCACATTCCTCGGCAGTATCCCGGGCTTGGCCAGCTTGCTGACACCTGGCTGGAATAACAATGAGCGATGCTTCTGGAGGAT
GCTCCGCTGGGGAGGGCTCTGCTCAACCAGCACTTACTCCCTGCTTTTCGATCCTCACTCTGCCTTCCTCTCTGACAAAGCCCCGGCTG
TTGGGATGGTAATGCCCTCTGGCCTCGCCTTGATTGAAGGTAAAAAATGAACTTCGAGGATAGCCCCTGTGGCTGTGCAGTCTCATTTT
TTATGCAGATATGTTGGGGGATGCAGAGACATGGTGGCGGGTGGGGTGGGGAGAAGCCCACTGGGAAATCTGAGACCTGAAGCTAAGAT
CTGAGATGTCATCCTTTATGGAGCATCTCCGGATGAAAATGTCCCAGACGCCCTACCTCACTCACCCTCTGCCCCCTAATAAAATCACT
GTTGCTGCCTCCTGCTGAATCTGAAGTATGCCTTCACTGAGGGCAGGACGTCCATCTCAACTATTATCAAAGGAAAACATAACAAGAGG
CAACAGCACAGCCCCCGGGGCATGAGGCTCCTTCCTCTGTGATCCCTCTGTCCTTGTCTGCCAGTCTTTTTTTCCTTGACTTTTAAAAT
AATCTTTTTCTTATTATGAAATACATATTTATTTTCCTTAACAAAGAATTCTGTAGTCCTTCTAGAAGCTAGGTAACTCTTCTAAGTCC
TTTGCAAATATTAAGTCATTTAATTTAATCCTAGAGCAGTTCTGCGAGGTGGTGGGAAGACCTAGAGAGGTTAGGGTCCACCTTAGCAC
TAGCTCTCTGAGCTATAAGGCCTCAGGGTCTGTGGCATTCCCTACTCACTGAAAAGAAGGGAATTTAAAACATGAGTTCCACCACCCAG
AAATGAAACAGTGTTCATTTCCAGAATCCAGGCATATGGTTGATTTTACCAATTTCCTATCACTTGAATCACAGTTTTTTTTGTTTGTT
TGTTTTTCTTTGAGATGGAGTCTCACTCTATTGCCCAGGTTGGAGTACAATGGTATGATCTTGGCTCACTACAACCCCGACCTCCTGGG
TTCAAGCAATTTTCGTGCCTCAGCTTCCAGAGTAGCTGGGGTTACGGGTGCATGCCACCACACCCAGCTAATTTTTTTGTATTTTTACT
AGAGATGGGTTTTCGCCATGTTGGCCAGGCTGGTCTCCAACTCCTGACCTCACGTTATCCACCCTCACATTATCCACCCTCCTACACCT
CCCACACTGCTGCGATTACACCTGTGAGTCACTGCACCTGGCCCACAGGTTTTTTTTAATAGTCCTGTGTGTATAATTTTGCATCCTAT
TATTTCTCCTTCCTTTTAACATATATATATATATATATATACGTATATATATATGTATATACATATATATAATATATGTATATACATAT
ATATATACATATATATGTATACATATATATACATATATATATATATATATGTATGTATTATCTCTTTACTTTTTAAAAGTTTCTCAGAC
TTTAAGAATTACTTAAAATTTCCCAAAACAAACCCACAGGCACTGCTCTGATCTTCAGGCACACTTTGGTATTCAGGAGTTGCTTCTGC
TGGACCAAAGTCAATAACAAGTAGCTTAAGAAGCTGTAGATGGCAGACATAATATTGACTGGAGGCTTACGCCTCTGGTTCAGGGTGGA
CTCAACAATTTTGCAAGTTCTATATTGTTAAAATTACAATGAATTATTTATAAGCAGTTGTTAGTAAATCATGAAGTTGGCATTATTAT
TCTTTATTTATTTATTTATTTTGAGATGGAGTCTCGCTCTGTTGCCCAGCCTCAAGCGCAATGGCATGATTTCGCCTCACTGCAACCTC
CACCTCCCAGGTTCCAGTGATTCTCCTGCCTCACCCTCCTGAGTAGCTGGGATTACAGGCATGCACCACCACACCCGCCTAATTTTTCT
ATTTTTAGTAGAGATGGAGTTTCATCATGTTGCCCAGGCTGCTCTCGAACTCCTCACCTCAGGTGATGCACCCACCTCCTCCTCTCAAA
GTCCTGGGATTACAGGTGTGAGCCACCACGCTTGCCCTATTTTTTATTATTAAAAAACAAAATGATCCTGATCCTTTCAAATATGTATT
GCAGTCTTATTACTTAGTATTGAGCTTTGCAGTGTTGGACTAATATCACAAGTCGAAGGGACAAAGAAGATGGTAGACAATACCAACTG
TTTCCCATTAGGCTCTTTCCCTCCTCTTGTTTTTCTGAGACCTTAAGGGCCTGGTTGTTTCTACTGTTCCCTGCTACTTCATAACCTGC
AACCTTGAGAATCTTTGTCATTTACAAATTTTACCTTATAGTAGAAGAACATACTAAATAATTGTACCAGATTGAGTAGTGTCCCCCCA
AAATTCATGCCTGTATGAACTTCAGAACATGGCATTATTTGGACATAGGGTTTTTGTTTTTGTTTTTGTTTTGAGATGGAGTCTCGCTC
TGTCACTCAGGAAGGAGTGCAGTCCCGTGATCTCGGCTTACTGCAACCTCTGCCTCCTGGGTTCAAGCTATTCTCCTGCCTCAGCCGTC
CGAGTAGCTCAGATTACACGCACCCGCCACCACGCTTGGCTATTTTTTGTACTTTTAGTAGAGACAGGGTGTCACCATGTTGGCCAGGC
TGGTCTCGAACTCCTGACCTTCAGTGATCCTCCTGTCTCGGCTTCCCAAAGTGTTGGGGTTACACGCACGAGCCACTGTGCCTGGTGGA
CATTGGGTCTTTTTAGATGTCATTACTTAAGATGATATCATATTGGATTAGGGTGGGGGCTAAACCCAATCACCCATGTCCTCAGAGGA
GAAGAGAGCCACATAGGAAAGAAGGATTTGTAGAGATACAGGGAGAAGGCCATGTGACCACAGAGGCAGAGATTAGAGTGACACAGCTA
CAAGCCAAGGAGCTCCAAGGATTGCAGGAAGCCACCAGATGCCAGAAAGAGGCAAGGAAGGATCTTTCATGGAGCCTTCAGAGGGAGCA
CAGTCCCGCTCACACCTTCATCTTGGACATCTAGTCCCCAGAACTCTCAGACACTCCATCTGCTGATGTAAGCCACCCAGTTTGTAGCA
ATTTGTTATGGCTGCCCTCAGAAACTAAGACAGTAGCCAACACAGAACCAAATACCCTTTGGGGATTGAGGGAAGTGGAGCGGGAGAAT
GAGCGGCACTCCCTCCCTTTACATAGGACTACTTGGTAAATATTTACTGAATGAAGCAAACCTGAAGTATCTGTACAACTGGCCATTTC
ATTCTGCATTTAGACCTCTGGGAGCTCTTGAAATGTCTAGACAAACATCCTCTTCAGCTAGAGGAAGCTCCTTCTCCCACTTACTAGCT
ATGAGATCTTGGGCAGATGGCTTAACATTTCTGGGCCTCAGTGACCTTACTTGCAAAGTTGTCTGGGTCAAGGCAATGATTAAATGCGA
TGATGCATGTAAAGCCCCAGCATGCAGCCTGAAAATGTCAGCCCTTATTATTTATACTGTTTGCCAAAGTGGTGAGTAGTGTTGCTGAA
TATGCCGTGTGTCCTATTAACTCTGTGACGGCCACAGTCACGTCTCCTTTCTGCCTCCTCCTCCTCCTCCTCTCTGTCCAGTGACTAGG
GACCGCACCAAGTTCAGAGGGTGAAATGCTCAATTAATTCCTCTCCTCCTGCTCTATATTCCAGATCACCCTCTGGGTGGGTTCAAAAC
CATGCCCTGTCTACCTCCTCTGCAAGGAAAAAATAAAGACAGAGAGCAAATCTAAAACACGCAATGCAGAGGAATGGTTACTGCCTGCT
GTGTGCCTCTCCAGGGAGCACCAGGAGGGGCTGTCTGGCACAGGGACACTGCGTATAAGTGACACTCCTCCACATTTTGGCCTGGACTT
AGCACATACAACTGCCTTTTTGCTGACGCGATGCCCAGGAGCCCTCTCAGGGGATGGCAAATCTCAGAATAAATGTGTTTTTTACTCAT
GGAATGCACGGGAATGGATATGAAAACTTTTGTATCAAATAGACATTAGCCAGCAGAGGGATGCTTCACATCCTAGGTTTGTGTGATGT
CCGCACAGCTGATCAGGTCTTTCCAAATTTTGCTCCTGAGGAAGGGATGCCCTGGACTCCCAGTGTCCAGGGGCCCTCTGCTTGTCTGT
CCTTAGGCAGAGTCCACCAGAATCGGGTAAAGTATGTGACTGCCTCTTAGTGTAGGGCATGACGTCAACTGTGAGGAGAAGAGATCAAG
ATACAGCTCTTTGGTGGTGTTTTTTGACCCATATCCAGGAACTGTTCCCACACCCAGAGAGGAATATCTGCAAGTTCTGGTGCCAGATG
TTGATTCAAAAACAAAACCCAGCGAAGCATCTGTACTTTTTTTTCTGATTGTATTAGAAGTACAGTCCACAATAATATATTGTACATTT
AAAAATAACTGGCTGAGCGCAGTGGCTCACGCCTGTAATCCCAGCATTTTGGGAGGCCAAGGCAGGCGGATCCCCTGAGGTCAGGAGTT
GGAGACCAACCTGGCCAACATGGCAAAACCCCGTCTCCACTAAAAATACAAAAAAAATTAGCCAGACGTGGTGATGGGCACCTGTAATC
CCAGCTATTCTGGAGGCTGAGGCAGGAGAATCACTTGAAGCCGGGAGGCGGAGGTTGCAGTGAGCTGAGATCATGCCATTGCACTCCAG
CCTGGGGGACAAGAGTGAGACTCCGTCTCAAAAAAACAAAAACAAAAGAACCCTCAAAAAACTAAAAGAGCCCCCGTGTGGTGGCTCAT
GCCTGTAATCCCAGCACTTGGAGAGGCCGAGGCAGGCAGATCACTGACGTCAGGAGTTCAAGACCAGCCTGGGTAACATGGTGAAACCT
TATCTCCACTAAAAATACAAAAATAGCCGGGCTTGGTGGTGGGTGCCTGTGACCCAAGCTACTCTGGAGGCTGAGGTGGGAGAATCGCT
TGAACCCAGGAGGTGGAGATTGCAGTGAGCTGAGATTGCGCCACTGCACTCCAGCCTGGGTGGCAGACACAGTATCCGTCTCAAATAAA
TAAATAAGTAAATAAGAGTTTAATTGGATTGTTCGTAACACAAAGAAAGGGTAAATCTTTCAGGTGATGGATGCCCCATTTACCCTGAA
GGGATTATTATGCATTGTGTGCCTCTATCAAAATATCTCATGTACCCTATAAATATATATGCCTGTGTACCCACAAAAATTAAAAATTA
AATAAAAACTTCATTAAAAATAACTTCAAACACTACAGAAATGTTAAAAGTAGAAGTGACCTTTCCGTAGAATGTCATCCCTGTACTTT
GGCAGAGAATTTCAATGGAAGCATCCAGAAAACCCTGGCTCGGAATCTGAGCTTGATTATTTCAGTGTTTCAGGTCTCACATGGGGGAC
AGTCTTGCCACTGTCAAAGGCCATCATGAGAGTTACAGCAGACTTTTGATGAAATTATGTCCACAGTGGTCCCTGGTCCTTTGTGGTTT
GAGATATATTCTTTGTCCCCACCCCTGCCCCTTGCTTCACATCTTCTAGGAATCCAGATACATTAAAGCAAAGTGACGGCACAGCCCTG
TCCATTTCCTGATCTGTTGCTGACTGTACCACTGAGCTGCTCTCTCCTACTGAGCCCCGCATGGTTCCTCCTGTTTTCCTGCCCTCTCA
CTTGCTGGTGTTGGCCTGCTCTGGCTGTTCTGCTTGGGGTTAGGTGAGGATATGGGGCAGAAGGCTTCCAGCCTGGAGCGCCTATGGTG
TCCCAAGACAAGGGCCTGCCTCTGGCCTCTGGGTGTGTCCTCCTGCGAGTTTTAGAGAAATCTGGGCAGGCTCAGACTGAATGCCTGCT
TCTCCTTGCAGCCTTCTGAAGATTCCCATGACACACACCTGATTTACCTTCAGCTCTTTTACTTGCTGGGTAAGCACAGCCCTGGATTA
CTTGGCATCTGGAATTAACTGAAGGGTAACACTTTCTTAACCTTATTTGCAAAATGAATAGGAAGGAGACCCCTTCTAACTTCTGGAAG
CACAAAAGTGTGTTTATCCTTCAGGAGCATTCTTGGCTGCTGAAAAATCATCATGGCTAGCTTTCACTTTCACGTATTGAGCTAGTAGG
TGGTATCAATAGTAGCTAATATCTATTTCATGTTTTTTTGTGTAGGAGGGACCCGGCGTGCAATTTGCATGCATTGTCTCATTTTATCC
TCCCAGCAGGTAAAAGAGCTGGGAGGCAGTAAAGGGAGTTGTTAATTTCTCACAAGGAAGGTGAAACTTAGGAATGTTAAATACCCCTC
TTGGTGTACACAGTATGCAATGGAGGCAGGGTGCAAATGTGCTATGGAGATTTCAGAGCCTGGGCTTCCCAGAGGACGCCTTTGCCCCA
CAGAATCTTTGCTCCATTTCTCCACTTTGCTGCCTCTATGAATCAATGATTCTGTGTTGTCCATTTTTCCTTTGCAATCTTTTGCCATC
TCTTCATTAAAAATGGTCTCCATCATTCTGGCCAAGCACAGTGACTAACATACAATAGGTGTGCAGTAAATGTCTAGTGGATGAATACC
CAGTGTCAACATTAATTGAGCACCACTAGCTGCCAAGCAGCCAAGCACCCTATATCTGGGAGATGTAGAATTTAAGGCTCTCTACTCAC
TGTCCTCAAGCTGCTCACAGCTTTGTGTAAGACACTCAAGGGAACATGCAAAGCTCAGGGCAGCTCAATTTGGGGCGAGGGTTATGGAG
AAAATACAGTCTGCATCCTTTTAGTACATGCAGTTCTGCATCTTGGGGTGCAGTGCTAGAGTGAATGACACAGTAGAGGTGACAGGCTT
AGCATTTAAATTTGATAATCTCATGATGCCATGGGAGCACTGGAGAAGGGGCACCAGTGGATTCAGCTGACACTCGCAACGCGTTAATG
AGAAGATTGGGAAAGGCTGCACAGAGGAGGAGAAGGGCTTTGTAGGATGAGTAGAGGTTCACCAAGGGTGGGTAACAGTTAGAGGAACA
GCAGGTACAGAAGCCCACACGAGAATCAGTGAATGACACATCCACCATGTCCAGAGATCAGGGATGGTTGCAGCTGAAGCTAAAAGTGT
AAGAGCTTGGACTGTGGATCTTCAATCCCACTTCATCTTATTCCCACCCATATCGCTGGTCCTATCCTTTTCTCACCTGTAACACAACT
ATGAGACTGGTACCTTCCTAGGAGGGGTGTTGAGAGTCTGAGCCACACTCAAATTCTTGGTGTGCAGTGTCTCTGATCTGAAATGTGCA
CCGTCAATGTTAGCAGTGGGTATTACCTTTGGCCTGATTGTGAAGCGGGTTTGAGACAGAAGTGAAGCAGTTCAATTTGTTGTGTGATC
TTCTTCTTCCCCAAAAGAAGGTTTTCATCCTCCTTAAGGCTTTTGCCCACCCTTCCCTTCCTTCACATCCCTGGTGGGTACCCTTCCCA
CCTGCAGGAAGCCTACTCATCTTTGAGTTTTGGCCTGCCACCTGCCCCTTCCCTCGCTCTGCAGTCTTGGTTGGCCTCCAGGTGTGTGT
GTGTGTGTGTGTGTGTGTGTGTGTGGGTGTGTGGCCCTGGGCTGCACTGTGCCTGCTGGAGCAGGTGCAGTTGCCCTTTCACCATGCGT
CCTTCTTTGGATCTGACCCCTTCGGTCCCTCTGTATTTAGAGTCGAGGAACGTCCTCCACTATAATCATCAGAAGTGTCATTGCTAGTT
GCACTCAGTTTTCTATGGTGTTTGGGGGAGAAATTGGGGTGCTGGATTTTGGTTTTAACTCTTTCCCACCTTAACCAGTGACTGTGGAA
AGTTGCACATCCTCCCCTTCCTCACAGCTGCGGTCTGTTCTTCCTCCGGGTGTTACGGTTTGTGAGAGCCACGAGCCTACACAGAGCTG
GCCTGATTTCTGGAGCCATAGCAAGAAGGGAGAGGTGTGTGTCCTCTCTGTCTCTGTCTCTGTCTCTGTCTCTGACTCTGTCTGTCTCT
GTCTCTGTCTCTCTCTCTCTCTCTCTCTTCTTTCTCCCTCTCCCTTTTTCACATGTTGTTTTCTGATTATAAGAGGAATACTTCATCAT
TAGACATATTTCGAGCCTCCGTTCCTCATCTATGAAATAAGCATAGTAAAGGACTGAAGGATTTAAAGGGATGTGGCCAAGTGTGCAAA
GTCATATCTGCTCAATAATTTATTTTCCTTGTTTTCTTTTCCATTTCCTTCTTCTCCTCTAAGCCCCTCCACATCTCCGTGATATAGCC
TGTACTCCTTTCCTCATTTTCTTTTCTCTGACACAAACCGAAGTTCCTACTTCTGCTATTTCCTAGTTCAGTCACCTTGGACAAGGCAC
CCTGGGGAGCCTTACCTGCTCATATGCACAAGCTTCTACCAACTGCTTCCAAGGGAAGCGTGGGCAGGAAGGGAGACCCTGAGTGTTTG
GACCCCGCCTGTGCTCAGCACAGAATGCAGCTCAGTAACGGCAGGTCGTCCGCTCCTTCTTCTCCCTTTCAGCAACCATCACTCGTATT
CTTTTTTATCTCCAAGGCCTCATGCTGGAAACAGATTCATTGCTCTCTGAGCAGTAAGTCCTCTGAAACAGGGTTTCTCTGCGGAGTTC
TGTGGCACCCCTGCCTTACCTGGGGGACCTGCCAAAGAGGCACCCTCCACCCCCCCTCACCCTCAGCCAGGTTTACTGAACAGAGTTAC
GGGGGTGGGCCTCCGGATCTGCATTGGAGCAAGTCCACAGGTGCTGCTTAGGCACACTCACCTTTGAGAGCCACTCACCTAGACGGGAA
GACTGGGAGAAGCCTTTTAACACACCACTACTGTGACTTGCACCATGGAGACACCTGGAAAACATACAGTGATTCACACACACGCCCCA
AACAGCAGTGTGTGTGTGTGTGTGTGTCTGTGTATGTGTGGTTTCACACTTGGTTTGCTATGAATGCTCTAATGCCACCCAGTTCCCAC
AGAGCTCTGGCGACTCAGCTTCCCCAAGTCAGGGTTCTTTTCTGGAGTCCTATTCTGACAGGTTTTTATTTTTATTTTTTTAATTATTT
TATTTTATTTTTATTTTATTAGTATTATTATATCTGACACGTTTTTAATACAGTGAATTTGTATGTCTCCTGTGTATTTATTTTAGCTG
CTCCCTTCTTTCCCCTTCTCTCCCTTTCCTCTCATCTCCTCCCGCTTTCGTCCTTTACCTTTCCCTCCATGATATTACCATTACCCGTA
CACTCTTCCCATCTCCTGGCCCCTCCAGTTCTGAAGGCATCGGCACTAAGTGGGATCTGTCCTCTAAGCAGGCGGAGGAGGGAACCATG
TCAGCTCCTAGGTGCCCCCAGAGCACCTGCTGCCAGTTTTCCACAACTCCTGTCCTCCGTGGCGCTTGGGTGACAGCAGAGCAGAGCCG
ATGTGCCTGCTCCTGGTCATGGGCTGTTCGTGCTGTCATCGCTTTCATTTGCCCCATCCCCCCAATTTGGACTTACCCAGAATCCTTGG
GCAACCTACATCGCTGCCAAAGTTTACTTAGCGAGATGTGCCTGCGTGACTTGAGTAGAGACCTGCTTCTGAGAAAGTCAGAAGTGCAT
TCCAAAGTCAGGAGCGTGACCTCCCCGGAAGCCCAGCTAATCCAGGTTTTAAAGAGGAGTTGCAACAAAGGTGGTGCTTATGGGATGTA
TGGAGCTGTGaGATCTGCCACAGGACAGGTTTAGCAGAGTGGCGTGGTGGAAAGTTTCCTCTCGCCGAGGAGGACAACTCCCCAGCGCC
TGAGACAGCGGGAGCTCAGAGCGCCTAGAATGTGCCCAGATTCTAAAGTGAAGCAGGCTGTGGTGTGATGATGGCCCCCGACGGCACTT
GCAGTCTTATGGACCTGGGTTCAACCCCACCCTCTTCAGCCACACAAACTGGGCAAGGGGCCCGCCCTTTTCACGCTGCTGTTTCTGTA
ACTTTCAGATATGAGACCTGTCTGTGGGGTTGTGAGGATACATGGGGATTAAGTGGGAAATTAAAAGGGTTTTCTTTGTGACTGGTGAC
TGATAAATGCCATTTCCTTTCATCCTCCTTATTTTGACATTAGCCCCAACCTCTACCCCGAGGGTCTCTCTCTCTCTCTTCCCAACTTA
GATTCCTCTCAGCGCACACATAGACAACCCAGAAAAGCAGCTCTGATCTCTCGTGGAGCTTCCCTGAACTCCACACCGCACATCTACTA
CTGTTTAGGATAACATTTTGACTTAGTCCTTAAACGGACTATTTCTCATAGTCCTGTTTTGTAAATCACATTTTAACCTCTTTGCAGAC
AGGGACCAGTATAAACTCTTGAGTCTCTGTCAAACTTAGTGAACTTTCTTTCCAGCCTGGGCAACATGATGAAAACCTCTCTCTACAAA
AAAAATACAAAAATTAGCAGGGCATGGTGGCTCGCACCCGTAGTCCCAGCTACTCTGGAGGCTGAGGTCGGAGAATTGCTTGAGCCTGG
GAGTTTGAGGCTGCAGTGAGCCATGATCACGCCACTGCACTCCACCCTGGGTGACAGAACCGGACTCTGTCTCAAAAAAACAACAACCC
CCCTCCCCCACAAAAAACCTTACTGAACTTTCTTGGAGAACATGCACACTGTAGAACCTTCTGTATTTGGAATATTCAAGTGCAGGTCC
TGCTGTTCTGTAAAGAACTGTCACCTTGAGTCATTGTTTGACGGCTCAGGTCCTTGGCACTTTACCCCTTTGTTCATGATTGATTGACA
TTTGGACCTTTGCTGACCTTTCCCTGACGCATTTTCTGGGCATAGTCAAACAAAAGGAAATTGAAAAAAAAAACCCCACAAATTTTGAA
GGTCTGTCACTTGGAGAAGAACAGTGTTCATGACTCTTCTTCGTCATTTCAGCATCTTAATCATCTTGAGCCTAAAGATGAACCAGAGT
GGAGGTGAGCCTGATTAAATGGGAGCTTTTCCTATATTGAGGATCTACTACGTGCAACGACCTCACTTGGGTTATCTCTAGCCTTCCCC
CAACTCTGCAAGGCATTTCACAGATATGGATGCAGGTTCAAAGAACTGAAGCTTAAAGCTTCGCAGATTTGGAAGCCTCAGAGCTAGAA
TTCTCCCCCTAGCCTGTTTGTCTCTGAAGCGTTGGTTATTGCTTGACATTGCATCCCTCCCTCTTTTTTCCCTCTCTTCCTCTTTCCCT
TCCCTGCCACTGTCTGCCTGTCTTCCTGTCTCCCTCTCTTTCTGTCTTAAAATTACTGTTGCTATTCCTTACTGGTCTGGTCTGGTCTA
GTTAATTAAAAAGAAGGCTATCGTGAGGTAACCCTGGGATCCTTATTGGCACAGTCATATTCCCATAACTTTTAGGTGCCTGAGTCCTA
ATGGATCTGTAAGACAGTGCCCATCAGCGAGAAATACCCACAGCCTCCCTGGATGCTGCCCACAGGCATCTTGCAGATGAGGGTGTTTG
CCTTCTCACCTGCAAGGGGGTGGGGAGAAGTGGCCAGCCGTCGCCTGTTCTGGGGACGCCTGCCTCCCCACTCCCTGAGCCTAGGGGGA
ATGATATTATGGCTTCAGGCCACAACTAGACACATTATCTACTCTGACAGGGAGCCAGTCTGGAGAAGGATGTCATGGATGACGTGAAG
AAATTACTGAGGGAAAAAGAGCAAGGCGTGAGCCCTATTGGGGCTGTTTTGGTCAACTCCTGGCACTAGATGATATTGATGCTTTGGAA
ATCTCTATTTTCCTTCCATTTCCCAGCAAAGTGCCATTCCCATCTCTTTCTTTTAGAAGCTTCTTATCCTATGGCTCTGCCTCCAGTCT
TTCTCATCACATAATTCTTCAAAGGCAAGGCAGACTAGTGGTTAAGAACAAAGACCCAGGGCTGGGCGTGGTGGCTCCTGCCTGTAATC
CCAGCACTTCGGCAGGCCAAGGCCGGGGTATCAGTTGAGCTCAGCACTTGGAGCCCAGCTTTGCCAACATGGTGAAACCCTGTCTCTGC
TAAAAATACAAAAATTAGCCGGGCGTGGTGGCAGGTGCCTGTAATCCTAGCTACTTGGGAGGCTGAGGCAGGAGAATCACTTGAACCCA
GGAGGTGGAGGTTGTAGTGAGCTGAGATCACGCCACTGCACTCCAGCCTGGGTGACAGAGCAAGACTCTATCTCAATTAAACAAAACAA
AACAAAACAAACAAACAAAAAAGAACAAAGTCTCCCGAGGCAGTGCCTGGGCTCGTATCCTAGCTCTGTCTCTTGCTGACTCCTGCATG
GCTTGGGCATACTTGGGCTTTGCTGTGCACCAGTGTTCTCGTTTCTTAGTGACTTCGTATCTCACAGCATTGTTGGAGATTACAGGAAC
ACAACCAATGCTCTCAGAACCACCACTCAGGAAGTGTGGTAATTACATGCTCTTCTGGGCTTCTGGTCATGCCACCTTCGACGGACAAT
CCGCCAGCACCCACCACTTTAAGGACCAGTTCCTCCTCTAGGCATTGTGCCCAGCGCACCCTTCTCATGGAGATTTGCTTCTCACTCTG
CATCTGCAGGTCTTCGCTGGGTGATCTTGGGCAAGTCACTTTACTTCTCCAGGCCTTACTTCTCAGCTCTTCAGTGAGGACATTGAACG
AGATTATTTCCAAGGTTTCTTGTGTCCTGACGTACTGCGGCCTCTCACATTCTATGTGGTCCAACCTTTCCTCCTGTTCCTCAGCTTCT
GCCAGCCCGCCACGGCAACCTGGTTATCAACAAATGTCCTCGCTTATGGACATGTCCTGCTTCTCTGGAAATCTTACTACACCCTGAAA
GCTCCATGAAGTCAGGATTGTGTCTTTCATGTTTGATATGATTTCTGGCTTCTAGCACTGTGCTAGGAGCTAGCACGTGAACACTGTCT
GTGTAGCTGGAATTAATCACCCACTATTCCAGTCAACAATTCCTTTTTTTTTTGAGACGGAGTCTTGCTCTGTCCCCCAGGCTGGAGTG
CAGTGGCATGATCTTGGCTCACTGCAACCTCCACTTCCGGGTTCACGCCATTCTCCTGCCTCAGCCTCCTCACTAGCTGGGACTACAGG
CGCTCGCCACCACGCCCGGCTAATTTTTTTTTTTTTGTATTTTTAGTAGAGGTGGGGTTTCACTGTGTTAGCCAGGATGGTCTCGATCT
CCTGACCTCATGATCCGCCTGTCTCGGTCTCCCAAAGTGCTGGGATTATAGGCATGAGCCATCGCCCCCGGCCCTTTTTTTTTTTTTTT
TTTTCCGAGATGGAGTCTCACTCTGTCTCCCAGGCTGGAGTGCAGTGGCATGATCACTGCTCATTGCAACTTCCGCCTGCTGGGTTCAA
CCAATTATCCTCCCTTACCCTCCCGACTACCTGCGACTATAGGCGTGCACCACCATGACTGGCTAATTTTTTGTACTTTAGTAGAGACG
GGGTTTTGCCATGCTGGCCAGGCTGGTCTTGAACTCCTGACCTCGTGATCCACACGCCTCAGCCTCCCAACGTGCTGGGATTACAGGCG
TGAGCCACTGCGCTCAGCTTTCCAGTCATTTCTTAAGCGATGTTCAGTTCCCAAGCACTGTGCCCGACACGCATGGGTTGCTGGACGCT
GGCAGAACCCGAATGTCAGTATTCCTGAAGAATGGCTCTAAGCAATGTACCTTGCAGAGCTCAAGCAGGGCTCTGCTGCATCATGCCCA
TGAAGGTGCCTGCTGGTAGAAGGCTGGTTCTATACTGGCTGCTTGCCTCCTCCTTGTGGAGACCCACATTGTGTCTCCCAAAGTGCAGA
AGGAAATCAAGCCCTTAATAACATTAGGGAACTGGACTGGGTTCGGTTTGCATCTCCCGAGTCTGACTCGTCGGAGTAACCACCATAAT
AGCAGCGCTGTGCTGTTGAGGGTGACTTTGACTATCTGCTTCTCTTGTGAGCCCACTGTGTGCCGGAGCACTCTCTATACATTCAGTCT
TGTCCGCAGATGAACTCCAGTGGACACATGTTTCTATTCCTTATTATTATTATTATCATTTGTTTTTCTGAGATAATAATAATAATATT
ATTATTAATATTATTGTTATTATATTATTATTATTATTATTATTATTATTATTTAGATGCAGTTTCACTCTTGTCGCCCAGGTTGGACT
GCAGTCGCACAATCACAATCTCAGCTCACTGCAACCTCCGCCTTCTGGGTTCAAGTGATTCTCCTGCCTCAGCCTCCCGAGTAGCTGAG
ATTACAGGTGCCCGCCACCACACTCAGCTAATTTTTGTATTTTTGGTAGCGATGGAGTTTCAACATGTTAGTTACCCCGGTCTCGAACT
TCTGACCTTAGGTGATCCACTTGCCTTGGCCTCCCAAAGTCCTGCCATCACAGGTGTGAGCCACCACACCCGGCCTATTATTATTATTT
TTTGAGACAGGGTCTTACTTTGTCACCTAGGCTGTTGCCTCACTGCAACCTCCACTGACTTCCTGGACTCAAGGAATCCTCTTACCTCA
GCCTCCTGAGTAGCTGGGACTACACGTCCGCCACCACTGCCACCTAATTTTTGTATTTTTTGTAGAGATATGCTTTCACCATGTTGTCC
ACGCTCGTCTCAAACTCCTGGCTTCAAGTGATCTCCCCGCCTCCACCTTCCAAAGTCCTACGATTACACGCATGAGCCGCTGTGCCCGC
CTTATTCTTTGTTTTATTGAACAGATGAGGAAACACTTTCAATGAGCTTAAGGGATGTGCCCAAGGCCACACGGTTGTGACCACAGCAT
GAGGATTCACACCTTGGTTCCTGTGGCTCCCATCCCCCCTCCCCTGAATCAGCAATCGCGTGAGTGTGACGGGAGCAGGCACTCCAACC
CTTGGGAATTCCAAAGCAGCTGATCCATGTCATCCCAGCACACGTTAGCGACGAACTGAAACCAAGCAAAAATAGAGCCTGTCAGCGCG
GGGACAGCTGCCGCTTTCTCTGGGAACAGATCTCCCCCATTTATCTTCCAGGTTAGGAAACCCCCAGCTCCTGGCGTACCACAGAGAAA
TAATGTGAGCACAGCCAGAGAAATTGCCACCTCTCCCTTCCTCAGAAGAAGATAAAGGAGACATCGTCTTTTTGGCATCAGTTTTGAAG
AAATCTTTTTTTTTTTTTGAGATCCAGTCTCCCTCCTGTTGCCCAGCCTGGACTCCAATCGCACAATCTCCGCTCACTGCAACCTCCCC
CTCCCAGGTTCAAGCGATTCTCCTGACTCAGCCTCCCACGTAGCTGGGATTACAGGTGCCCGCCACCACGCCCAGCTAATTTTTGTATT
TTTAGTACAGAGGGGCTTTCGCCATGTTGCCCAGTCTCCTCTCGAACTCATGACCTCAGGTCATCCACCTCCTCGGCCTCCCAAAGTGC
TCCCATTACACCCGTGAGCCACCACACCCAGCCACTTTTGAACAAATCTTACTTCACAATCCTCACAGAAAGAGAAATTGAGGCACACT
TAGCATGTGAGACTGATTTCTCACTTCTGCTGCTACAACTGCTGCCCCATTGACCGGGTCTCAGAGGTGTTATAATTAACATCCTCTGA
CTCTTGCTGGTACTAAAATTAACAACATGACTGGTCCCTTCTGCAAGACAGGGAAAGAGATACACACATGTGCACATACACGCACACAC
ATGCCTGCACACACACACTTGCACGCACACATGCATGCACACACATCACACACACACACACACACACACACACAGACACATGCACAGTC
CAGAACGCTCACAGACAGAGAGAGAGAGACGAACGCACAGCCACACACACACTCCCAGACACTTATTATGGCTCCCCCTGCAAACGGTG
CTCCCCTCAAAGCTTTAGGACCAGGGACGCCTTCCCTCACTTTCTGTGTTCTGTACCTCATACCCCATTTACCAGCCTGTCCCAACCCT
GTATACTTTAAGTCTTCAGTTACCAAAGCATCCATCTGCCCGTCCTATATTGAGGGGGAGTGGATGCGCAGAGGTGGTCTGTTGGTGTT
GCGTACGCAATAGACGCACCCTGTTTTTCGTTTTTCTTCCAAATGTCTTCGGCAGGGCCTCACCTAGTGCAAATACCTGGTGGGGAGGG
TCAAGAAGGAAATGACAGTGGGGGGAATGGGGAGGGATAGCATTACGAGATATACCTAATGTTAAATGACGAGTTAATGGGTGTAGCAC
ACAAGCATGGCACATGTATACATATGTAACAAACCTGCACGTTGTGCACATGTACCCTAAAACTTAAAGTATAATAAAAAAAAATAAAA
TTAAAAAAATAAAATAAAAAAAGAATTCCTATTTTAACTTTGGAAAAAAAGAAAAAAAGAAGGAAATGACAGATTTGGTCCTGACAGCC
TGGGACGCAGAGAGGAGAGAGATGGAGCCAAGAGAATGCGGCTGGGGCCACGTTTTCTTGTGGCGCAATTCCGTGTCACTGGGCCAGAG
CATGCTTATTGTGAAGACTTCCCCTCAGTGTCCTCTGGGCAAGGCTCACCTGTGAGGCAGTCGCCCGTCAGAATACCATCAAACACGGA
AACCTGGAAGCTGCCGATTCCCAGGCCTTGAAGGGGTGTTCTGATGTCTTTGACATTCCAGCAAACATATTTTCACAGGAAAACTCAAA
AAATATAAACTCCCTTTTTATAGAAATAATGTGCATGGAGAAAAGTGCACATGTCATATGTGTATCGATAGCTTGACGAATGTTCTCAA
ACTCAACCTGAGTGTGGAGCCAGCACCTGGATCAATAGAACATTTCCAGCATTTTGGAAGCCCCCTCCCAGTCAGTCACTCAGGAGAAC
TTTGTCCTGACTTCTAACCACACAGATTAGTTTAACCTGTTTATGTACTTAATCTAAATAACATCATCACACACTCCCCTGTGTCTTGC
TTCTTTCACTCAAAATTATGTTTGTCGGATATATTCATGTGCTACCTGCATTTTCAGATCATTCTCACTGGTCGCTAGATTGTGTGCAT
ATTTTAAAAAATCCATTCTACTGTTGATGGGCATTTGAGTAGTTTCCAGTGTTTGGCTGTTATGAATAGAGCTGCTGTGATATCCTTAG
ACTTGTCTTCTGGTGAACATATGCACACATATCTGTGCATTGATACCACGGAGTGGAGCTGCAGGGTCATAGGGTTTGGGTATATTCAG
CATTAGTAAATAGTGCAGAACAGTTTCCCAAAGTGGTTGTTTATCCTTTTTCAGAGTGTCTGTTCACCAGCACGTATGAATTCCAGTTC
CACATCAAAGCCAATTATTGGCATTGTGTGGTTTTTAAATTTTAGCCAACCCATTACAGAAAGAGCAAATGAAAGGAATCTAACAGCAA
GTAATCAGGAGAGAAAAGAAAATGCTCCCTGAGAAAGCATTATGATTTCATGATACGCCAAATCCCGGTGTACAATGCCACACATTGTG
TACTTTTGCAGGCGGAGGGCGAGGTGCAGCCACCTTCTTGTGAGCATCAGGGCAGTCCTTGCCATTGGTCAGGGACCACCACTTGACTG
CAGTAATACCCAACAGACCATGTCATAATTCTTCCTCAGGTGACAGAGCTTTAATGGATCCGACCCCAACAGAACAAACTTGGCTTCAG
ACTTGCCTGTGGGCCTCAGTAAATATTCAACAGACTGTGTCCCCCTGTTCACTCCCCTGTCATTAGCTGACGCTGCTACCATAAATCAC
AGCTCCCCTTCCTGTTGTAAGTGGCCCCTCTCAGCTGACGGTACTCCAGTCCTGACATTCAGGAGCAGTGCCTCTCCTGGGCTGCTTCC
CAGGGCTGGCAGGTCTTGGTGGTCACTTGTAGGAGCCGAGGACTGAGTCTGTGCAGTCCTGGAGTCACTGCTGTGGGGTGACTTCCAGG
AGCCTCTTCCATTTGATGTGAGAATGATAGATCCGTCAAGCTCTTAATATGTGCCCAGCAACATGCTAAGTCCTTTATGTTGTTTATTG
CAACCTGTCACAGTCCATTCAGGCTGGTATAACAAACCACCTTAGACTGGTAGCTTGTAAACAACAGAACTTTATTTGTCATGGTTCTG
GACGTGGGCAAGTCCAACATTAAGGCTCACACATATTTGGTGTCTGGTGAGGGCCTGTTTCCTGGTTTAGCAATGATGCCTTCTTGTTG
GGTCTTTATATGGTGAAAGGATCAAATAAGGTATTTTATACCTCTTTTATAAGGGCACTAAGCCCTCCAGCCTCATGACCGAATCACTT
TTCAAAGGCCTCCCCTTTTGAGATCATCACCTTGGCAGTTAAGAGTTCAACGTATGAATTTAGGGGTCGGGGGTGTAGGTAACAAACAT
TCAAAACATCATACAACCTCACACTACTTCTATGAAATGAGTAGATGTTCTTTACCACCATTTTCAGATGAGGAAATTAAGCGTTAAAA
ACATTTCCCCAGAGTAAGAAACGTAGTAAATTTGGGAGTCTAACTTCAAACTGTAATTTTTAATTACTGTACTTCCTTCTTCTTTTCTC
TTAGGCGCTGTTTAGATACTTGAGCTTCCTTTTGAAGCCATCTTCCTCTTCGGTCTAAAAGCTTTCAGGTAGATTTAAATGGGACAACA
AGGCCAGATGCAGTGGCTAACACCTCTAATCCCACCTATTTGGGAGGCTGAGGCAGGAGAATCACTTGAACCTGGGAGGTGGAAGTTGC
AGTGAGCTGAGATGGCACCCTTCCACTCCAGCCTCCGCAACAGAGCAAGACTCTGTCTCAAGGAAAAAATAAATAAAATAAATAAATAA
ATAAATGGGACAAGAGGAAGTCACCTCCAGACCACATATTCCGTTCACATGTAATTATCTGCTTTTTCAATGATTTTAACAAATTAGAA
CCACTGTTTTTCTCTGGCTCTTTTCATGACTTTTTTCAAATGTTGGTTTTCAGCAGTTCAACTATTATATACTTAGGTGTGATTTAAAA
AAATATATATCCTGCATGGGATTTGCTGATATTCTTATATCTATAAATTAATGTCTTTCACTGAATTTAGAACACTTTCATCCCTTCCC
ACCCCCGATATTGATCTTGCTCCATTCTCTTCTCTCCTTTTGGGACTAAAATTACATGTCTGTTAGGCTTTTTGATATTGTCCTACAGG
TCTCTCAGGCTCTCTTCAATTTTTCAAATTTTTTTCTGTTATTCAGATTAAATAATTTCTATTGGCCTCTCCTCAAATTGTACTGACTC
TTGGTTCCTCTTCAGTCTGCCGTTAAATGGATCCAATTGATTGTTTCAGATTTTTTTTTTAATTGTAGAATTTCCATTTGGCTCTTTCT
TTGCTAAGCTTTCTTATCTTTTTATTTACTAAGAATACATTTTCCTTTGAGTCTTTGAGCATAGTTATAATAGCTGCTTAAAAAACCCT
TGTCTCCTAATTCCAGAAACTGGGTCATCTCAGGATCATGGGGACGCTCCCCATAACTATCTTTTTTCTTAAGAATGGATTACATTTCC
TGTTTTTGTTTTCTTTTTAGTTGTTTGTTATATTGAGCTATTATAAATTATATCACACACATTGTGAGTGATACTTCGTAAGGACTGAT
TCTGTTTTGTTCCTCCAAAGAGTAGTGCTTTTTTGTTTCAGTAGGTAGTTAATTCAGTAATCTAAGACTTCAAATTCTGACTCCCTAGT
ATTCGCCAGCAATATAAATTGCTGTTTAGTTCTTTTCGCTTTAACTCGGCTACTCCATACAAACTTGGCTCAGGAGTCACTTAGTGACT
TAGGGAACATTTGTACACAGAATTTGAACTCCCCTTTTTGGAAACTCTCTTTTTACCAGGATTTCTTCTTTCACTTTCACCTATTGTGT
TGCCTTGATCTCTGTCCTGATGTTTCAGTGCAGCAACACCATCTGAGTGTTAGCCACTCACATGTTGCAGAACCGGGCCTACTCTCAGA
CTAAAATTGACCAAAAGCACCAACTCACTCGCTTGCTGAATGTCCAGGTCTTGATCACACTCCAGAATGTCCTACTTTTGGTCATCAGG
TGCCTAGATTTTTTAGAAATCTTTGTTCTAATTTTTACAAATATTTGTTCAGAGTTTATAGTTGCTATCTACCAGGGAACTGCTTCAAT
AGGAACTACTCAACCATTGCCAGAACCTTCTTGAACTTTCTAGAAACACTGTCTTATTTATTTTATTTTATTTTTTAGACAAAATCTCA
CTCTGTGCTCTGCCACCCAGGCTGGCGTGCAATGGTGTAATCTTGGTTCACTGCAACCTCCACCTCCCGAGTTCAAGTGATTCTCGTTC
CTCAGCCTCCCAGGTAGCTGGGATTACAGGCACCTGCCACCACACCTGGCTAATTGTTTGTATTTTTAGTAGAGACGGGGTTTCACCAC
GTTGCCCAGTCTCGTCTCAAACTCCTGACTTCAGGCGATCCACCCATCTCAGCCTCCCAAAGTGCTGGGATTTACAGGCATGAGCCACC
ACGCTTGGCCTAGAAACACTGTCTTATTTAAATAAGAAAGAGTCTCTCTCAGAATGGAAAGAAAACACCTTTCGAGTCAAACCCAACTT
GGATGCAGAATCTCACTCTATCTTGGTTTTTTTTTTTTTTTTTTTTTTTTTTTGAGACTCAATCTCACTCTGTCACCTAGGCTAGAGTG
CAGTGGCATGATCTTGGCTCACTACAACCTCGGCCTCCTGGGTTCAAGCCATTCTTCTGCCTTAGCCTCCTGAGTAGCTGTGATTACAG
GTGTGTGCCACCACGCCTGGCTGATTTTTTGTAATTTTAGTAGAGACGGGGTTTCACCGTGTTAGCCAGGATGATCTCGATCTCCTGAC
CTCGTGATCCACCCGCCTCCCCCTCCCAAACTCCTGGGATTACACGCCTGAGCCACCGCACTCCGCTGCATATGTTTTTTAAGAAGAAA
ACGTACCTTACTGTTTCCTTCCTGATTTCTTCACTAGCCGCTGCAAGTGTGTGTGGCTCTGTGCATTGTCCGCAGGGTCACGTACCTAC
CTTGATTTCCATTTTGGCAATGAGTGGTGCCTCCTCATGCAAGACTTCTTAAAACAGCAGATCGGTGCCCTCTCTTATTCTTGTATCGC
AGCCAGAATAACCTCCCCCTAAATGTCCACATCCGAATCCCTAGAACCTGCGAATATGTTGCATGTCAAAAGGGACTTTGCAGATGCTA
GTAACGCTAAGCACCCTCACGTGCACCCATCATTTTGGATCATCTCCGTGAGCCCAATATAATCACATGCGTCCTTAGGACTCGAACAG
GGAGGCAGAGAGTTATTCAGAGACAGAGATATGGCCACAGAAGCAGACTCACACATGCCTTGGTGTTGTATTTGAAGATGGAGGAAGAA
GCCACAGCCAAGGCCTATGAAGCAGCCTTTAGAAGCTTCCAAAACCAACCAAACACATTCCCTCTCAGCCACCAGAAAGAAATGCAGCC
CTCCCAACAGCCTGATTTTAGCTCAGTGAGACCCATGTCAGACTTCCAGAAATGTAAGATCATAAGTGTGTGCTCTTTTATTTTTTATT
TCTTTTTCTGTTTTAAACCACTACGTTTGTAAACATTTCTTTGGCAGTAATCGTTGCTTTTGATGGAAGTTGGAGCAGTGGTCAACCTC
GTCCCCTTTGGAGCCAGTCTCCCACTCCGCCTTCTTTCAAACCCCTTTTCCTTTCTCTCTTTCTTCCTTCCTTCCTTCCTTCCTTCCTT
CCTTCCTTCCTTCCTTCCTTCCCTCCTTCCTTCCCTCTCTCTCTTTCTTTCTTTCTTCTTTTTTTTTTTGAGATAGAGTCTCATTCTGT
GGCCCAGGCTCGAATGCAGTGGTGTGATCTTGGCTCACTGCAACCTCCGTCTCCTTGGTTTAAGCCATTCTCATTCCTCGCCCTCCCAA
ATACCTGGAATTACACGCATGCACCACCACACCCAGCTAATTTTTGTATTTTTAGTAGAGACAGGGTTTCACCATGTTGGCCAGGCTGG
TCTTGAATTCCTGGCCTCAAGGGATCCACCAGCCTCGGCATCACAAAGTCCTAGGATTACAGGCGTGAGCCACCGCCCCCAGCCTCAAA
CCCTTTTCCTGTGATTTTTTTTTTTTTTTTTAAAGCTGTGTGACCTTGTGCAAGTTGCTTCACTTCTCTGGCCCTCAAATTTCTCATCT
CTAAAGTCGGTTAACAATAATCTACAACTCATAGTTTTTTGTGAGGATGAAATGAGGGTTTCATACATGCAGAAAGCCTGGAAGTAAGT
TTTAGCTGTGGCCATTCTGGGCAGGAATCCCTCACTACGTAACTTAGAGCGGCGGATCAGGGCACAGACTTGGGACACAGACTCTCTAG
CTTCAGATTTAGGCTTCTTACTAGTCTGAAGTAGAATGGTTGTCTGTTCCTCCACTGCTTCATCTGGAAAACAGGGGCAGGCATCCCAG
GGTGTTTGAGAGATCATATCAGATGGAGAGGCCAGAGTTTTTAGCCGATACATCATAGACATTCCCCAAAGGACAGTTATTGTTATCAT
TCGGTGGATGGGGAATATTGAGCCTCCAGCCCTAGGCATTGTGGATCAATTTGTTCTTCAGTTATCTTTGGAATAATTTAAATACACTG
GATTGGGAAGACAAAGCTATCACCCTCCTGATGGGAGAGTGCTGGGTACCTAGCAGGTGCTCAGTAGTTAGTGAATGAATGAGTAAGAA
CACAACACGTCCTAGGCCCTGCCCTGAACAGGCTCACAGTCCACCACAAGGCCTGGAGTCAGGCCAGGGTTTTGTGGTTGTCTTTAAAA
AGAAACAAAACAAAGAACTACTTCCTACCAGCTGACCCTAAACCCCTCAGCTCCCTGCAGGGACCTCTCAGCCCTGCAGGGGAGCCCAG
GCTATGTGCCAGGCCTGGCCTTCCTGTCAGCTTTCTAAGCCAGTCACAAACATTCTCCTCTCAACTTTATAATGGATGCCCCCCACCCT
CCTCTCCCCTCTCAGGGTGCTCCTGAGGCTATGAGCAGCTGGCAGTTCCCTGTCTGAGAGGTCCTTCGTACCGCAGGAGAGGCAGCTCT
CACCTGAAGGCCTCCCTGGACACCGGTCTCAACTTTGTGTGTTACCAGTTATGGAGAATTATTTTTAGTGAGTTACACTCTGTTAGGGC
CAAACACAACGAGCAAAGAGTCACTGAATACTGCTCTGACTCAGTGACTGTGACAAGACTGTTTTTTTCTTCTTCCCTGTTGCAGGCCT
GATCTTTTGGCCAGAAGGAGATTAAAAAGATGCCCCTCAAGATGGCTGTGCCTGTCAGCTGCATGGAGCTTCGTTCAAGTATTTTCTGA
GCCTGATGGATTTACAGTGATCTTCAGTCGTCTGGCGAATAACGCTGGTGGAACCATGCACTGGAATGACACACGCCCGGCACATTTCA
GGATACTAAAAGTGGTTTTAAGGGAGGCTGTGGCTGAATGCCTCATGGATTCTTACAGCTTGGATGTCCATGGGGGACGAAGGACTGCA
GCTGGCTGAGAGGGTTGAGATCTCTGTTTACTTAGATCTCTGCCAACTTCCTTTGGGTCTCCCTATGGAATGTAAGACCCCGACTCTTC
CTGCTGAAGCATCTGATGCACGTTCCATCCGGCGCTCAGCTGGGCTTGACCTGAGGCTGTCCTCCCTGCTCACGGAGGGTTCACTGGGA
TGTGGCTGGTGTTCTCAACGAGGTGGCATTTAATGAGCACCCTCTGTGTGGTTGGCGTGATGCTGGGTGCCTGGGGGAGACAGATGGAA
CTAAAGAGGTCCTTCCTGGAAGCCGTTACTGTTCCCTGCCCTGACGTGTGTTATTGTACCTGTCACCATTTTCTGATGTTCCCAGTTTA
CGTATCTGTCACCATCTACCTCCCTATAAAGTCCCTCTCCTGTTGGTTCTGTGTCCTTAAGACATTGTGTGGCATTTGGCACAGTCTCT
CATGCTGCAGAAAGGGThAATTAAGACAGGATTGTTAATGAATGAAGACATGGGAGATTCAATGGAAAGATATGCATGGGGGGATAATT
TCTTGGCTCTGTGCTCAGAGCTAGGAACAACATAAGCCCATATGCCTGGTGCTGTGAGGGTTGTATGTGGGCAGATCCATGTCGACCCA
GACAGGCCTCGGTATCACTCTTTAACACATACACACTCCATGCCTCCAGGCAGACTGATGGATTCCTTTCCCTGCAGTGAAGGGTTAAG
TATTATCACACGTCACAGGACTGGAATTGAAGATCGCCAAGTAGACATTGAAATGCTGACCACATGACAGACACTCAGCATGTGGTGGG
TGATGTTGGCTGCGGGTAGCTTTTGGGGATGGTGGACATTTCTTGGCCCTGATCTTTCTGAGCAGAGCCAGGGAGGCAGTGCTGCACTC
TGACCTGGGCACAGCTGGTTGGCGTGGACAGTTGATTAAAGGTCACAGTTTCCCTGCAGCCCATGGGAAGGCAGAGTGACAAGTCAGTG
GCAGATGAGGAAATGGAGTCTCATGCCAGCTGGAGGGAGGGAGCAGCTGGGTTGGCCACAAAGGCCTGTTGCAGTTCTGCAGCTGACAA
GTCGAAGCACTGGCCTCTCAGGTCCAGGCCTGCTGCCCTGCGACCCTCCTAGCTCTGAGCGATGAGTGGTGCCAAGGTGCCTGAAGGAG
AGTTGCAGCTCAAACAGCAAGTGGTCCAGCTCTCAGACCACTTGAGGCCACCTGGCATGTGGCGGCCAAGACCACAGTTGGGGCGGGGC
CTTGAGTCTGATTTTGATTGGATTCCTAGTTCGGCTCATGCCTGACAGCACTTTCTCAGACAGGCTTCCCTGACCTCCTAAGGTAAATA
ATGTCCCTCTGGGTGTTACATGCTGTTCTCACCCATAGCTCTTAGATGTGTTCTAATTGTATATTCATATGTGCAATTTACTGGGTTTC
CTCTCTTTCTCTCACACTAGAAAGTAATCTTTCTGAGTTGAGGAAATGCATTTGCCTTGCACACAGGCTAGACACATACATAGTAGGCA
TTTTATTTGTTGCATCACAGTTGCCTTCATCAAATATGGTGACTCTCCCTGGTTTTCTCTTGGTTTCTTTGAAAAGGTCAGACAGAAGG
GAAACACTCTTTTCTTGAGTATATATATGACTTAGCCCAGCAATCCCACTAGGAGAGCAATGGTTTATCCATTATACAGTTGAGCGCCT
GGAGGCTCTGAGGGGCCATGTACCTTCCCCAAGGTTATTTATCCAGGAGAAGACACACCCAGAACCTCACAACTGCCTCGTGTTTTATA
CCAAACAAGGCCCCTTCAGCTGAAGACTTTTCCTTGCGGTCTTGGGAAGGGTGTGTGTGACGGTGAGCTGCAGCAGGTGCTGGGCTGTG
CTTGACTGTGGTCACGTAGGGGCAGGCAGAGGAGCCATTTCGGGGGGCTGCTGAGGGTCTGGAGAGAGGCTCACAGTGGTGAGCAAAGA
CCTTCTGGGATGGGAGGGGATCAGTTGGCAAAGACAGAGGCAGATACTCCCCACTCCTTTGTGCCTTCCCAGTCCTCTCTCACCCCTTC
CTGCCCTCTCCCCACACCCTATCGTGGTGGCAGCTGTGCCTCACTTATCACATGGTCCATAGCTCTGTTTTTTCCCCTTTTATCAGTTA
CTGCCTAGAACAACCCAATTGTGGTGGTCTGGGTTATCTCCCACTCTTCACAGTAGGGCCAGAGTGGGCTCAGACACCAAACGACACAG
AATGGTTATGGCAATGAATAAGATTCCTTCTCTGTGTCAGCAAGGATGCTGATGTAGTAATAATCTCCATTTATTAAGCCATGCTGTCT
ACAGGGCACATAGCACATGTTTTTCCATTCAAACCTCAAAACAGAGCTATGAGATGCGCACCATTATGATGATTATCTGTTTTACTGTT
TTTTGTTTTGTTTTGTTTTTGTTGTTTTTTTTGAGACTGAGTCTTACTCTATCATCCAGCCTGCACTGCAGTCGCCCAATCTCGGCTCA
CTGCAACCTCCACCTCCTTGGTTCAAGTGATTCTTCTGCGTCAGCCTCCCAAGTAGCTGGGACTACAGGCGTGCCCCACCATGCCTGGC
TAATTTTTGTTGGCCAGGCTGGTCTTGAACTCCTGACTTCAAGTGATCTGCCTGCCTCGGCCTCCTAAAGAGCTGCGATTACAAGCGTG
ACCCACCTTGCCCGGTCTGTTTTACTGTTGAAGTAACTGGAGCACCCAGAGGGAAATCAAGTGTCCCTAGAGAATACAAGCACCATTTG
GACAGCCCTGTCTGACTCCAAAGCCACTGCACAACATTGCCTGGAGCTGAGAGATGCACATTCTCCCTTCTTGGCCACTGGTGACTGTG
ACCTTGGGCAAACTACCTCTGCTTTTTGGGCCTTAGTACATTCATTCATAGAGAAGCTGGCTAATTTCTCTTCTCAGCCTGGAGACTTG
AGAGTGAAACGGGCACTGTATCACTTACTCTGGGATGACAGGTGTATACGAGGATGTAGCTGGGCAAGCCACGCCTACTGGCTCGCACA
CTCATCAGTTAGATCCTCGGTCCTGGTGCTGCTCGTCCATCATCCATACAAGGCTGGCTCTCCCAGTTTTCCCTGGACTTTTGCCTGAA
CAGTTTGCATTTCTACTCGTGGTGCAGGAGCTTGCTGTTCTGATTTCATGTCACAAGCCTGCTTTCAGCCACTTAGAAGAGCCCTGTTA
TCCCTGACTGCCGCTACAGCAGTCCCAACCTGTCTTGTTTTCACTTGATGGGCTCCTGCAATTATTGACACCACACTCTCTACTCCTAC
GGGCTGCCCTGCCACCTACACTGACATCCTGGCCAGGTTCCTGGAGATGGCCTGGCATCTTGGACCTGTTCCCCACGGGGTGCCTGGAG
CCTCTGATGGTCTACCCAGGGTGGCAATGAGGGAAAGGCTGTGACTTGATCATCTCCAGGGGTTCTTCGGTGAACAGCCCAAGGTTGCT
TAACAGCTGGTGTGTATGAATACCTGGAATGTGCCAGTTCACCTTCTAATCTGGCAAGGAGAATGTGTCTCCTCCTTATGCATATGAAC
AAACTGAGGTTCAGTGAGATCCAGAAAGCTGCCACGGTTACTGATGGAGAGAGCAGAGAAGCTGGTGTTTGCAGTCCCATCTCTCAGCC
TTCACACCCCTACTCCTGTCCAGCCAGTGTTTCTCAAACCCTCCTGATCAGCAATGCAAGATGATTTCATGTTATAGATAAGAATAAAA
AAATTGTTTTCTGTTTAACTCAAATTACAAAAAGGCAACAATTGGTATGTGCGACCTGTGGTTTTGCAGATGATACTGCTTACGATCTT
GGTACTTAACAAAAGGTCAACTTTTCAAAAATACTATTAGTGACATGTGGACCTAGTCCTCCTGAACAGGACTACATTGGGGCACCGGT
AATTGTTTCTATTTGCGGTACTCTCCCTGTGTCCCTCTGGCCACCCCACTCGACGCAGTGTCTGAGCCTGTCACTTGAGTAGTACCTCT
GTGTCATGTCTGCTGATTCTCCCCAAATCCTGAAGATTCATGATGAAGTGACTCGCCGGCTTGGTCTGAAGCTAGATTGAAAACAATAA
GGATCCCAGAACGATACCACTTTACAATCCTATAATTTGCCTCAAATTGCCTGCAGTTACTATCTCAGCCCTGCCTGTTATGTTCATTG
AGCACCCAAAGTTTTTCAGTCAATTCCTGAGTTAATTATTCTCTGCCATTCAATTATGAAATAGTAAATATTTCCACTATGCAATCAAT
TGGTGACTTATTCATGTATTCATTTCATTCATTTAGTCTCAATAAATTGAAGATAAGGGCTGAGCACAGTGGCTCACACATGTAATCCC
AGCACTTTGCGAGCCCCACCTGGGCAGATCACCTGAGGTCAGGAGTTCAAGACCAGCCTAACCAACATACCGAAACCCCATCACTACTA
AAAATAGAAAAATTAGCTGGGTGTGGTGGCATGCGCCTGTAATCCCAGCTACTCAGGAGGCTGACGCAGGACAATCATTTGAATCTCGG
AGGCGGAGGTTGCAGTGAGCCGAGATTGCACCACTGCACTCCAGCCTGCGTGACAAACTGACACTCCATCTCCCAAATAAATAAATAAA
TAAATAAAACATAATATTACCTACCCCATGACTTTATTATCACAATTAAATAAGAGAACATATTAAAAGGTTTCATTCAGTGCCAGGCA
TATAATATGTACTCAGGGAATACTAGTTTTTTTTAAATAAAATTTTAAAATGGGATTAGAAGGTCAAAGCATATACGCATAAAGGTATA
AAAAATATTGAAGATCAGTCAGTTACTAAAATTTAACATTACGTTTAGCTCTGAGCTTCCTAATTAGCACAACATGCTAACTAGGTTAT
ATTTCTATCTAAACTAAAGATTGGCAAACTTTTTATATCAAGGGTTAGGTGACAAATGTTTTCAGCTTTCCAGGCCACACACCTCTGTA
CTGCTTGTCAGTTCTGCCTTTGTATCTGGAAAGTAGCCATACATAATATGTAAATGAGTGGGTGTCGCTGTGTGCCAATAAAACTTTAT
TTACAAGAACTGGCAATAGGCCTTTAGGCTGTACTTTCTCCTTGGCCTAAATAAAGCAAACATGTTTGTCTTTCAAAGGCAGAAACTCC
TCCTGGATCATAAACATTGAAAAAAAAATTGTTACAAGATGCAATATTTCTGTGAGACTTGTTAAGCAGTATATGCGCAGTGCTTTCAT
TAGGATTTTACAGAAAACTTAGAAGATGGCCTTCACATGGCTAGTTTCCTTACTGTTCATTCACCAGACATTTACTGAGGGCTTACTAT
GTCCTAGGCTCTGATATCCATACATGGTAAAACGTAAAGGCATGTGCATTTTGGCAGGGGGTGCTGCATTGACAAGGAGTGGTCTCGGT
GACAGTTTTGGGAAGTCAGTTTAGAACAGTATTGGACACACTGTTCCATCCCTGAATTACACACAGGCCTCAATCCTAACTTGAGTGGG
CTTTTGGTCCAGCAGGCCTGGGCTCCAGGGGCAGTCACTCAGCGTCCTGCCTCAAGAGTGGTTGTGTCATGAAATCTTGGCCCTCTTTG
ACTGATAGCTACAATTATTTGAAGAGTGTGGTTTTTAGAACAAAACTAAATCTGTTTTGAAGCTCTGCTGTTTTATACCTGCTGTGTGA
TCTTGCTCAAGTTTTTATTTTTATTTTTTTCTGTATAAGGGATTTTATTTTATTTTTATTTATTATCTATGTATTTATTTTATTTTAAG
TTCTGGGGTACATGTGCAGGATGTGCAGGTTTGTTACATGGTAGACGTGTGCCATGTGATTTGCTGCATCTATCACCCCATCACCTAGA
TATTAAGCCCAGCATGCATTAGCTATTTTTCCTGATGCCTTTCCTCCCCCCCCCCGCCCCCGACCCACAGGCCCCAATGTGCTCAAGTG
TTTTAATCTCTCTGAGCCTCACTTTCTTCATCTGCAAACCATAACACCTCTGAATCTATTTGTTACTCTAATGCTAGCTATCTAGTGTT
TGCTCTCTAAATGTTAGTTCCTCCACCTGGGGGCTGGGCGGGCGGTGGCTGACCTCACCCCATCAAGCTGTCTCCTGGCTTTCCCAGCT
ACAGCCTGTCCGTATGAACATTCCAGCCCCCTCCTCCTGCGGCAGTATTCAGTGTAACAAGCCTTGAGAAAACACTGGCCTGTGGAATT
CCAAAGTTAATGAAGGGAACAGATTCCAAGAGGGTCTCTGTTTGACCTTCCACTCTTTCCCCAACCCCGTTACCCCTGAGCGAGTTTGG
GGGCAAGCATAGGTTTGTAGGCACTGCAAAGAAGAACTCTCACGCTGTGGTGCAGGACCGTCGCCTTGGCCTCTGGATATCTGGGCCAG
GTGGGTCCATTTGGGCCTCTTCCCTACAGCGCCTGGTCAGACTGCAAGAGACATAGGAAGCAGCCAGGGCAGGCTATCTTAAGAGGAGG
GAGCATATTTGATAAAGTTAGAAAATGATTCTCAGCTGTCCATAGGAAGAGCAGAGTAAGGGGCTATGTGAGAAGTTTGGAAGATGGCC
CAGTTCCAAAAATGAAGAAAGAAAGTAAGAAAGAAAGAGAGAAAGAGAGAAAGAAAGCAACCAAGGAACGAAGGAAGGAAAGAAAGAGG
AAAGAAAGAAAGAGGAAAGAAAGAAGGAAAGAAAGAACGAAGGAAAGAAAGAAAGAAGAGAGGGAGAGGAAGGAAGGAAGAAAGGAAAG
AAAGAAAGAAAGACAGACAAAGAAAGAAAGACAGGAAGGAAGGAAGAAAGGAAAGAAAGAAAGAAAAAGGAAGGAAGGAAAGAAGGAAG
GAGGGGAAGAAGCAAGAAACAAGGAAAGAAACATTCATTATTCTGTCCTCTGAACCTGAGTGAAGAAAAATACCCTGTCCTTTGTACCT
GCGTGAAGAGAGAAAAGAAAAGAAAAGAAAAGAAAGAAAGAAAGAAAGAAAAGAAAGACAGGAAGGAAGAAAGGAAAGAAAGAAAGAAA
GAAAGAAAGAAAAAAAGACAGACAGGAAGGAAGAAAGGAAAGAAAGAAAGAAAAGCGAAGGAAGGAAGGAAAGAAGGAAGGAAGGAAGA
AAGAACGAAAGAAAGAACATTCATTATTCTCTCCTCTGAACCTCAGTGAACAAATATACTCTGTCCTTTGTACCTGCGTGAAGAAAGAA
AAGAAAAGAAAGAAAAGGGAAGGAAGGAAGGAAAGAAGGAAGGAAGGAAGAAAGAAGGAAAGAAAGAAGATTCATTATTCTGTCCTCTG
AACCTGAGTGAACAAATATACTCTGTCCTTTGTACCTGCGTGAAGAAAGAAGAGAAAAGAAAGAAAAGAAAGAAAGAAAGAAAGAAACG
AAGGAAGGAAGAAAGAAAGAGTGTAAAGAAATAAAAGGCTCTAAAGAAATAAAATAATGTAAAGAAAGAAAATAATGTAAAGAAAGAAA
AGAAAGGAAAGAAAGATTCATTACTATCCTTTGGACCTGAGTGGTGTTTTCAGTTAAAATGAGTGGAGTAGAACCCAGAGATCAGGCTT
TTGACCACGGTAAACCCTCAAGATACATTCTGTTTCTGCCTCAGCGAACCTTCACGGAAAAGGCTTTGGAGACCAAGCTACGCAGGGAA
GCGCCTTTGCTTATCATGCTGGAGACCAGACTGAGGCAGTGAAGTCACCATAATTGATGGGCCACACTAGACTGGAGCCAACCATGGCC
CATTGCTGAGACTCTGGGCCTCAGGGTGGCCTTGGCTGGAACCTGTATTCCAGTGGCCCTGGTGGCTCTCTTCCCTCTCACCTTCCCAG
GAGGCAGATTATTTCCCTGTGGTGGGGGTGAGGCGGTGGCGTGGGGGCTTCTTCCCTCTCTCTCTGTCTTGTAGCCTTGTTAAAATAGT
GGCCACACGCTGATAGCTGGATTTCCAGTGCAGTTGCTGGGGATGGGGGTCTTTCCCTCTCATCTGTCTGTCACCCTTTATCACTCCAC
AAGGGGTGCGCGTACTGCACACAAACACTGTGTCCCACCTGATTACTGGGAAGTGCAATGGACACCCCCACCCCCCGTTCACTTATCTG
GCTCCAACCGAAGAACAGCTGTTGGTGGTCAGGTCTCTGAGTCACTCAGTAGCTTGGGACTGGGTTCAGTGGGAGATGATGGCATAAAC
AAATAAACACTGGCTGGCCCCCAGCTCTCAGCTTTCTGCTCACTGGGGGGCCCCAGAAGCTTCCTGTTGCGTCTGCAGGGAAAATAAGC
ATACCCATAATGAAAATAGTTTTCCTGATGGAGAAATGCTCTGGGGAGGTTGTTGAGGCATGTTACAAAATCCCTGAGGCTTTCTGGCT
CTGTGAGCTAAGGGAGGACACTTGGCCTCTCTGGGCTTTCGCTTGCTCCTTTGGCAAGCCTGTTATTGTGAAAGTGAGATGATCTGGGA
GGTAAAACTGGCCAGTTGTCCATCTCCCCTCTCTGAACATCATGCATAGTAACCCCTGAATCTCATTTAAATTTGGAAGTCAGACTGAC
TTTGTGACTGTCCTAGTTTCCGGCTGTTGAGTCAGCTCAGGCAGTTCTTGGGACTTCTGTGAGTCTCTGGCTCCCCGTCTCTCAATGGG
GTGACAGTCCTCGTCTTGTGCCCCTCACGCGCTTGTTGAGATCACAGATGAGTCACCTCTGGGAAACTGCCTGAGAGCTGGATGACACC
ACTCTGTGGGATTCCATTTCATCCACAGAGACATCTGGAAAAGGAGGCAGAGCAGAGCCTGACTTTTGATTATTTGGGGAGGGAGCCTG
GAGCTCGTGGAAGTGAGGGCATAGAGCACATTCCTTTCAGGCCAGGCAGTGCTTGGCTGCTGAGGCACCGGTGGAGACCTCATGATCTG
GTGGTCCGCCTCCCTCTTGTGCTGTGTGTGCCTGGAGCTCTCTGGCTTCCTTGGCTTTCCACTGTCTTTCCCAAAGCCTCCATGTATTC
TAGAATCTGCTATGCAGTTTCACGGGGATCTCTGTATAGTGTGAATATCTAGCTCTGAAACACAAAAGAGAAAGCTACAAAGCCCAACG
TAACAAAAGCCGTATTCCCACCCAAGGATTAGCACATGCTCATGAGGTGGCATGCTTTGCTTTGGATCATTGTCTTTATTTTATTTTAT
TTTGTTTATTTATTTATTTATTATTTATTTTTTGAGATGCAGTCTCGCTCTGTTCCCCAGGCTGGAATGCAGTGGTGCAATCTCAGCTC
ACTCCACCCTCTGCCTCCCAGGTTCAAGCCATTCTCCTGCCTCAGCCTCCTGAGTAGCTGCGACTGCAGCCGCGTACCACCACACCTGG
CTAATTTTTTATATATTTAGTAGAGATGGGGTTTCACTGTGTTAGCCACGATGGTCTTGATCTTCTGACCTCATGATCCCCCCACCTCG
GCCTCCCAAAGTGCTGCCATTACACCCATGAGCCACCACACCCAGCCTGGATCATTGTCTTTAAAGAAATTTAAAAAAAAAAATAGATA
AATGCGAACTCTATGACCCTTGTCTCCCATTCCCCATCCTTCTCTTCATACAAGTACCTCTCCTGAACTTCCGGTATAACACGTCATGC
TCTTACAATTTCATTACAAATCTATTTGAAGATCACTGGTAGCACCATTTTCTGTGTTTTTAAATTCTTAGGTAGCTGACATAACGTAT
AAGATGTCTGCACTTTCCTCTTTTCTCCCGGCGCTATGCTGGCCAGATGATGCTACTATGCTTGTTCATTCATTCCCTGGGCCACAGAG
TCTTGGGTTGTTTCCAGAATTTTGCTTTTGTAAACAATCCTGTCATGAGCATCCTAGCACAGGGCTGCAGGTGCATGTCTGGGTGTGTC
CCAGCCACTACAGGGCACACAGCATGCACATTTGCAGCCTGACTGGAGGTTGCCACATCACTCTCCAGTTGGCACTATTTCCTAGGTTC
CTTCCCTCACAGCCTGCCAACTTCTGCAAGCCAGCAGTGGGTGGTCTTCACCACTATATACCTCTGCCAGTCAGCATCCCCCACACCAT
GTACCAGCTCATGACACGCCTGAATGGGGTGAATTCTCGAAGCGGACAAGGCAAGTGCCAGCCCAGCTCCAGACTCTGGGTGTGGAACG
ACCAACATAATGCGCCCCCCTTGCAGAACCAGCAGAAAGAACAAAGAAATAGGCGCCTGGAGTGGGGGTGTGAATGAAGACATTGCTGT
TGAGAAGATACTTGTGAACAACCCAACACCGTTGTCTGCCCCTGGCTCCATATCATCAGGCTGCACATCGAGGTCCCAGGCACCATTGC
TGTGGGCTTTTGGGCAGTTGGATGCACAGTGGAGACCTGAGGAGGAAGTTCCCCTTGCTGCACACGCCTGCTGGAGGGGCTGGCTGGAG
TCTGGCTCTGGCCCTCACTCACTGAGGGTTATCCAAGTGACCCATTGCAGGAAGAGGGGTGGGGGACCCAGAGTTACTGTAACCACTTT
ATGAGTCTGTCTCAGCCACCATCTGCGCCTTCTACCAAACCCCACACCACAGGCACATTTTACCAAACCCAAGATTGACATGCCCTCGC
TGGCCCAGAAGGACACTTCAGGGCAGCAGGGCTGTAAAGGTCAGGAGGGGGAAACTTCTGGAAGCAACTTCAATCTTGTGGGAAAAAGC
TGACATAAATGAACAAGGAATGATTTTTTTCTTTTCTTTTTTTTATTTTTCTTTTTAGAGAGACAGTCTTGATATGTTGCCTAGGCTGG
TCTCAAACTTTTGAACTTGAGTGATCCTCCCACCTTGGCCTCCCAAAGTGCTGGAATTATAGGCATGAACCACCATTGTACAGCCACAA
GGGAAGATTAAACATAGACAGTTTGAGAAGAGTTTTTTTTTTTTTAAATTTCTAATTTTATATTTAATAGAGATGCAGTCTCACTATGT
TGCTCAGGCTAGTCTTAAAGTCCTGACCTCAACCGATCCTCCTGCCTTGTTCTCCCAAAGTGCTGGGATTACAGGCTTGACCCACTGCA
CCTGGCCAAGGGTACTTTTTTTTTTTTTAAGAAAAATATCGTTAGAAATAGAAAATTTGCATTTGGTAATGGATACTGGATAGTATGAA
ATAACCTTTTTACTTCTTCAACACTTCCCATGATAATAACACTAATACAAGATCTTTGGTGAAAACTTAGAATGCGTACAAATGTGTTG
GTGGTTGAAAGAGAGCCCCATAACCCTCCACTGAGGAGTAACCTCTGTTTGACCTTAGAAATAATACCTGTGAACAGAATGGCCAACTT
GTCCTGGTTTGCCAGAGACTTTCCTGGTTTTGGCACGGAAAGTCCAACACTCTGGGAACCTCCTCAGTCCTGTGCAAACTGGGATGGTC
GTTCACCCTACCTGTCAATTGAATGATACCACTGAGCATCAAGCACTTTGACAGTTCACAAGTCATTTCTAGTGCTGTCATTGTGTATA
CTTGGTTCCCCCTGTGTCCAAGGCATTGTGCGAGGCTGCATGTTCTGGAGCTACACCAGAGCTCCAGAGCTCAAGTTCTATTTTCTTTT
TTTTTTTTTTTCACTTTTTATTTTAGAATCAAGAGATGCATATGCAGGTTTGTTACAGAGGTATATGGGATGCGGTTGAAGTTTGGAGT
ATGATTGAACTCATCACGCAGGTACTGAGCATAGTATCCAGTAGTTTTTCAATCCTTGTCCCCCTCTTTTCCTCCCCTTTCTAGTAGCC
CTCAGCATCTATTGTTCCCATCTTTATGCCCATGTGTACCCAATGTTTAGTTCCCACTTATAAGTGAGAACATGCGGTATTTTGTTTTC
TGTTTCAGCATTAGTTTGCTTATGATAATGGCCTCCAGCTGCATCCATGTTGCTGCACACAGCGTGATTCCATTCTTTTATATGGTTGC
ATAGTATTTCTTGGTGTATATCTCCCACATTTTCTTTATCCAGTCCACCACTGACGGGCATTTATGTTAATTCCATGTCTTTGCTATTG
TGAATAGTGCTGCCATCAACACATGCGTGCGTGTGTACTTTTCGTAGAACAATTTATTTTCCTTTGAGTATACACCCAGGTATGAGATT
GGTCGGTTAAATGGTAGTTCAACTTGGAGTTTTCTGAGAAATCTCTAAACTGCTCCTCACAGTGCCTCGACTAATTTACATTCCCACCA
ACACTGTGTCAGTGTCTCCTTTTCTACACACGCCCACCAACATCTGTTATTTTTTGACTTTTTAATGAAAACCATTCTGATTGGCATGA
AATGTATCTCATTGTGGTTTTGATTTGAATTTTTCCAATGACTAGTGATGTTGTGCATTTTTTCATGTTTGGTGGCTGCATGAATGTCT
TCTTTTGAGCAGTGTCCGTCCTTGTGCTTTGCCCACTTTTTAATGGGATTATTTGCTTCTTTGCTTTTTGATTTAAGTTCCTTATCGAT
TCTGGATATTGGAACTTTGTCAAATGCATACTTTGTGAATATTTTCTCCCATTCTGTAGCTTGTCTGTGTACTCCCTTGATAGTTTCTT
TTGCTCTGCAGAAACTCTTTAGTTTAATTAGGCCCCAATTGTCAATTTTTGTTTTTGTTGCAGTTGTTTTTGAGGACTTAGCCATAAAT
TCCTGTCAAGGCCTATATTGACACGGGTATTTCCTCGGTTTTCTTCTAGGATTTTTATAGTTTGAAGTCTTAAGTCTTTAAGCCATGTT
GAGTTATTTTTTGTGTGGTGATAAATAGGGATTCTGTTTCATTTTCTTCTGCATATGGATAGCCATTTATCCCAGCACCATTTATTGAA
TAGGAAGTCTTTTCCCCATTGCCTCTTTTTGTCAAGTTTGTTGAAGATCAGATGGTTGTAGATAAGTGGCTTTATTTCTCGATTCTCTA
TTCTGTTCCGTTGCTCTGTGTATTTGTTTTTGCACCAGTACCATGCTGTTTGGGTTACTGTAGCCTTGTAGTATAATTTCAAGTCAGGT
AATGTGAGGTCTCTAGCTTTGTTCTTATTTCTTAGAATTGCTTTGGTCATTCAGCCTCTTTTTTGGTTCCATGTGAATTTTAGAATAGT
TTTTTCTACTTCTGTGAAAAATGATGTTGATAGTTTGAAAGAAATAACATTGAATCTATAAATTGCTTTGGACAGTATGGCCATTTTAA
TGATTTTGATTCTTCCAATCAATGAGCATGAAATGTTTTTCCATTTATTTGTGTCATGTCTGATTTCTTTCAACAATGTTTTGTAGTCC
TTGTAGAGATCTTTCACCTCCTTGGTTAGAAAGTATTACTAGATATTTTATTTATGTGTCTATGAAGCCCTGTTTTCTATCCTTAAGGA
GCTTACAACTTCTCTTTGGTATGCAGTAGCCGCATTTCACTTGTGAGAGTGGGGGTCTTACTGTAGCCTGGGCAATACCCTCTTTTATT
ATTGGGTACTAAGGATTTAAGGATATTTTTGTTCTGGTGGGTTATCCAGTTTTGCAGGTTGGTACCAGGAGAGAGGATGCATGTTGGTA
CCAATGCTGAATTTGACTGCCCCTGGCCCTGCTGTGCAGCACTTTCAGACCCCTAAATGATCTCACCCATTCTGGCGCCAGCTCAGTGT
TTTACAGTCATGAACATGATGTTGATGATCATGATGATGATGTCAACATCCATCATTCTGAGGTGTGTGTCTATCTCTGGCCTCGTTAT
ACCTGCAGAACATTCATGTGGTAGCCAATACCTGGGAAAATAATCCCACTTCCCAGGCAGCAAACCGTGGCCCTCCGTTAGGCGGATGG
CATCTCACACTCATCAAGTCTTGAGTGTATGGGTCCCTGGCCAGCTTTGGCAACTGTGAGGCAGGCATTATCCTTCCATTTTACCACTC
ACAGACTGAGGCCGGTGATGGGGAGCACCTGCGCCTCTTGGCTTGTCTGTGGTAGCCCACGCTGGCTACATAATATGTGGGGCTCAGTG
CAAAATCAAATATGGGCTCTCTTGTGTAAAAATTAGGAAGAATTTCTAGATGCTGACACCACAGCTTTAAACCAAGCTCGGGCCCCTCT
GAGCCAGGAGCCCTGTGTGCCTGCACAGGCTGTATGTCCAAGGAGCTGGTCCTAGGGCAGGACTAAGGTTCAGGCTTCGTATTGACTTC
CATGACCAAGCATCCTGGGTCCAGGCTGTGTTTTGTGTGTCTCCACAGCTCTCCTCTATCTGCTCATAAGTCCCTCTCGTGCTCAAAGG
TACCTCGCTTCCTTGCAGAAAAGTTTCTCTCTCCCCTGCCCTTCCCCAGCCTCAAAATACACAAATGCTCAGATGAATACTTAACAATC
CAACTGCACCAGCTTCTGTCTGTGACTTCATCACTTAATGTGATTTTGGGGGATGCAACATTGGGGGTGATGCTTTAGCATGCCTTCTC
TCTCCCACTCAGGAACATGGGCAAATGCCATGGGCTCGCAGCTCTCACCTGCTCTTTTCTGCTGAGCTTCACTATCTCTTCTGGGCACA
GATTGGTTACAGGGATATAGACCACCTGTGTCGTGCCCTGGGGGTTATGTCCACGGGGAGCCTCACATGAAGAAGAGAATTTCCTTTGG
GATGTGTATTTCTTGTGGTCTTAGCATGCTGGCAATTCTTATTTTTCGCATCATTTAATCCTTACAACAACTTTGTAAGGTTTTCACAT
CCATTCGTCATTTCTCAGTTGAGCACCTCAACTCAGGTGAATGTGTTTGTATGAATGTTTGTTGAGCACCTACTATGTGCAGGGCAGTG
TAGTAAAAAATTGAGGACCTGTGCCTTTTTTTTTGAGTATATATAATTCTGTTCTTCTGTCATTTTACATGACTTTCACCTCATTTCAT
TGCTGTAGCAACACTGTGAGGTTGTTGCTGTCACCCCCATTTCTCAGGCGAGGAAATAGAGGTTGAGTCAATCTCCCATCTTGGATTTA
CCTGACATCACAGTCTGAGTCTTTTGTGGCATGTGCAAGAGTGCACTGGCTTTCGGGGCCTGAGCTGGCTGGCCTGGATTGAATCCTGG
CTTTGCCATTTCCAGCTGTGGTCTGAGGCAAGTTATTTAACTTCTCTTGACCTCGGTTTCCCCATCTCTAAAATGGGGCTATACTAGTT
GCTCTGTGTAGGAAGGGTCTGAGGTTTCAGTAAGATACATTTTGCACAAACCCCACACACACCAAGTATCCATCAACCCTGGTTATCCT
TATAACCTCACAGCGGTTTGGAGAGGGTTACGAACTTGCAGATGGTCACATAATCGGTGTATGGCAGATCTTGGGCAGAGCTGGACTGG
TCTTCCTAATCAGAGCTCCTTTCCCCACTCTGCTTTCATGGACAGACCCTAATGACCACCTGGGTTATGTTTTTGTCCCTGTCAATTCC
TTTGTCCTTGCTGATAACCTAAGGGAACCTTGAGAGCCAGAACGAGACAGGAAGAAAATGCCTGGCTGCTCCTTGCGGGGTTAATTAAA
CCATTCCTGGGGTTTCATTTGCTCTCTAGTTAGCACCAAAATAAATCCTCCAAATGCCTTTGAGCCTTCCAGTTCACATTGCATTTTGA
ATTCTCACAGCACCAGCATTTTACATCTTCCTGAGCATGATAAATGTTTACATACCACCGAAGATAATGGTAAAAGGAGAAACTGGGGG
TAGTTCAGGCTGTGGCTGAGCAGATTGTAACTCTTTGGTGTGACCAATCGCTGACAAGTTGCTTAAAGGACACATCTGATGTCCCCTGC
TCCTAGCCCTTCAGAGGGGGATGGCGGGTCGACTCTGTTTTGCCATCCGGAGAATCCATTGCCTTCCCGATGCTTCTGGGGTCTCATGC
TTGAGTCTTGTTTCCATGCGTTCTGTCAGATGTTAAGGTCTTGTGCATAATGGTTGAGGTGTCAATGTCCAGGCGTTAATGTCACCATT
CTTATCTAATTGTTACCCTTTATTGAGTGCCTACTATGTGCCAGGATTTAAATATTTCTCTCAGCAACCCAAACATGTAGATGTCATAT
CACCTTCATTTTACAGATGTGAAAACAGGCTCGGACACGCTGAGACACCCACCCATTAAGGACTCAGCTGGGATTCCCAGCCTTCCCTG
GCACTCTCTCTTTTTTTTTTTTTTTTTTCTGTTTTGAGACACAGTCTTGCTCTGTCACCCACGCTGGAGTGCAGTGGCACAATATCAGC
TCACTTCAACCTCCCTCTCCCGGCCTCAACCAATTCTCCTGTCTCAGCCTCCCGAGTAGCTGGATTTGCAGGCATGTGCCACCGCACCC
GGCTAATTTTTATTTTTAATTTTTTTTGGTAGAGACGGGGTTTCACCATGTTGGCCACGCTACTCTTGAACTCCTCACCTCAGGTAATC
TGCCAGCCTCGGCCTCCCAAAGTGCTGGGATTACACCTCTGACCCACCATCCCCGCCCTTCCCTGCCACTCTTCAGCATGTTTCCAGAC
CGCCTGCTTCCTACCCCAGACATGTCAAAATGCGGTGCTGTAAATGACAGGGCTCTGGTCCCAGATGGACACCCCCGGCCCTTTGAAGA
CACTCTGGCCTTCAGGAGTCTGGATCTTTATCCCTCATCGAACTGACACCGGCCTCCTATGTTTACAGTGCCTGTTCTCCTGCAGCCTC
TCCGGAAGTCATCAGCCGTGCTGGCCAGCACTCCGGAGGATGACAGGAAATTCTGCTGCAGCTCTGTGGGCTGGCTGGGGTGCATGCTG
GACAGGGACCCCCCCCACAGGGCTGGTAGGGACGAAAACACCACTGAAATAGTTCAGACCGTATTGTCACTGAATTCCTCCATTAGTAT
GCAGTGCTGCCAATGATCAGATTTGCCTCACGAAGCCCAGATGGCACAAAGATTCTGTTAAATCTTTATATTTTTCCACCAAATCCTCA
CCGTTTCAGACCCTGTTTTCTAAGCTATCACCCTAATAATTGTGGATTTTGGATTTCCAACCCACGTTTTATAGTGGTTGTAAGGAGGG
GATGGTTGGGTTGGGGATTGGGATGGGAGGGGGGTAGCTGAAGAAATAAGAAAGTGCTGCAGAAAGGAGCACATTCCACACCCACCTTC
TTCCTGATTGTGTTTCCCTCCAAGCAGCGAACAAGACCCTGATTCAACTGGCTCAGTGGCCTCCCTCCCTGGAGTTGGGTTCCTATGCC
TTCCACTCACTAAGGAGGAAATTCCATTTCAGTTAATCTGTGGCCGCGCATCACCCATGCCATTCTCTATCTTAATTTTATGGCTGCCT
CATCTTTTTGGAATTCGCCTTCTTTTTGTGTGTTGGGTGATAAGTCAAGGACACATATGGCACAGCTCCACAAAATACAACCACATTTT
AGTATTCCGTACACAGTCCTTTCTGGGCATCCCACCCTTTGTATTTTCACCTTGGTCGGCCGTGAGCACCGTGGATCTTCATTATCTGT
GCATGCATGTGCATCCCTTCATCCCTTCCTCTTCACTTTCTTTTCTTTTTTTTTCTTTTTTCTTTCTTTCTTTTTTTTTTTGAGATGGA
ATCTTCCTCTGTCACCCAGGCTGGAGTACAGTGGCCTGATATTGACTCGCTGCAACCTCCGCCTCCTGGGTTCAAGCGATTCTCCTCCC
TCAGCCTCCTGAGTAGCTGCGATTACAGGGTTGTGCCACCACGCCCGCCTAATTTTTGTATTTTTAGTAAAGACGCGGTTTCACCATAT
TGACCAGGCTGGTCTCAAACTCCTGACCTTGTGATCCGCCCACCTCAGCCTCCCAAAGTGTTGGGATTACAGGCGTGAGCCACCACATC
CAGCCTCCTGTTCAGTTTCTTAACCCTTGATGGGACTCCCGGCTAAACACCCCCATTGTCTGCCACCTTGAGATTACATGCAGTAGCAC
TGCTGGTTTCATGGTGGACTTTGAGTCGGATGGAGGAACTCCTTGGAGTGCGGGAGGAAGGGCTGCGGAAGGTGTCTGAACTATCGTAG
CAGTACGGTCCCTGCCACAGGGAGGTGAGGCAGCCATCATCACCACTCCTAAGAGGTACGGTCACCCTCCGCCTCTGTCTCCCTCTCCA
GTCCCCATCCAATTTCACCTTTGAACCCAGCTGCCGGGTCTTCAAAGCCTGTTCTGACTTACCCAGCACAGTTGCTTGCTCTCTCCTCT
TAGCTTATAGACGCTGCCCGTTTTTTCCCCACGTGGCCCCTGCCATTTAGGTCCATGTCTCTCGTGATCCTGTGTGTATTTCCAGTGGA
AGGACAAGGTTTGGCCCACTGTATATTTGAAGGCCCTCAATATGGCGTTAGTTCGTATTTACTGATGGATGAAGAACTCTGAGGAGATC
TGGTTGAAAGAGACTTTTAAAAATTAAACGGAAATGGGTCACGCAGGCCGCGTTTTTGTCCTTGCCTATTGGCTACTTTTCCTATTCAG
ATCATCTGTGAATGTAAGTGACTGTCTGCTGTGTTCGCAGTGTGGTTACTGGGAAAACAGAGCTCTCTCTCCCCAAATCAGGTGAAACT
ACAGGAAAACAGTAGGCAAGAGTCTTCAAAGTGATGCATCTCGAGAGAGAGAGGGCCTGGGTCTGAATCATGGCTTTGTGCCTTACCAG
CCTGTGATTCTAGTCACTTTACCTTCTGAGCCTTGTTTTTCCCAGCTGCAAAATGAGTGTGATAATAGCGCTCGCTTCAGGGGATTGTT
TCAAGGATCAGTTCAAATAGTGCACACCGAATGTTTGGCATATGTGCGGAGCTCTTTGGGCGTGACTCATGGGCAACTGTTTTTCATTT
AAGAACTGAGTGGATTGGACCTTTAAGAACTCCTTTGGTCCCACTGTCATGGGAGGTTAGTGCTGTCTGGAGCAGGCATGGGGATGTCG
TGGTACAGGGTAGGGTGTAGGGGTCCCCAGAATTGGGGGCCTCCCTCTTCGAAAGGGACAGTGGGCAGAGTCCATGCAGGCGCTCTGGG
GCTGACAGGGACCTGGGACTTCTTTCTGAGAGGCAGTGACCAGCCAACTGGAGTGGAGGGTTGTGGGGAAAATGTTTGGCCAAAGGATT
TAGGAACCAGAGAGAAGCAGGCACATGATGCTCCTTTTCTTGGTGGGGTGTTCATCAGGCCTGTAGCTGCCACAGTCATCACTGTGGCT
GAGATTCATACCTGTAGCACCAGAGTGGGAGGCAATTTTTCCAACATGGGGCAGAGGCTGGGTGTGTGGGGATTGGTTGCAAGGTGCTG
GGAGTGGGGTGCAGGGAAGCGCCTCTGCACACCACGTCCGTCCTCATCCTGCTATGAGGTCAGCTCTGTACATGTGCTGCAAGAGCAAT
TAGCGGGGTGCCTAAGCAGTGTCCATCATGGACGTGGGTCTCTGCTGGCCACGCAAGGGGAACATGGGACTGGTGGGAAGATTGGGGAG
ATGGAAGCGGGGGCAGTTGTGTGCGAAAGGTACAGGGTCGTGGAAACATGCACAAGAAAGTGACATTTTAGAAGGTGGTTTTTGTTCCA
TAATAAATTTATTAGCTTTGGAATCTGACTCTATTGCCTTGGCTTCTCCAAACTTTGGTGTCCTCATCTGCAAACTGGGGATCACAATC
TCAGTTTTGCAGGGAATGTGAAATCCTGACCTGAAATGATCACATGTGTAGGAAGCTGACAAAGAATGCTAATTTTCTTCCTTCTTTTC
TTTATGGCCTTTCCTTGCTAAATCTCTGTGTGATTGATAACTCATGAAGAACCTACCAGTCAAATCAGTTAAATTAAATGACTTCCTAA
TGATAATAATTAGATTAGTAACTAATAATCATAGCTGACATTTATCTAGTGCTTATTAACTTCTGGGCAGTGCGAATTTGGTCTCATCT
GTTAAATAATTTAATTCCTGTAATAATCCTAGGAGGATGGTGCTATAATTAGCCCCATTTTACAAACAAGAAAACTGGAGGATCAGAGA
GGCTAAGTATAATGTCTAATCTCACACTGCTGAAAGTGGCTTGCTGGTGTTCCAGCCCCAACAGTCTGGCTCCAGAGTTTGTGCTATTA
ACCATTATATCAGTCTGCTGTGTGTCTGTGTGTTTGTCTCTCTGTGTGTGTGTGAGAGAGAGACAGAGAGAGATTGTGCATTGTACGGT
GAAGAGCAATACAAATCATTATAAGGCATTCCTGTGCTACAGTCTTGGTGGGTCAGGCTTACTTATAGAAGCTCTGAGAAGGATGGGGC
TATGGGCTTTAACTCAGTTCTGACAATCTGTCACTCCCAGAACCTCAGGCAGGCTGGACCTATAGACTTCCACCTCCTCTTCCTCCTGC
CCTCAACCGTCCAGGGACTCAGAGGAGGTCAGACAGGTGGGACTCCCACAATAGTGACCTCCTCTCCCACTTGCAGCCTATGTCGGGCT
CCTCTTATAGAGCCACCCCCTTGCTTATGCTGTAGTGTTCCTTTGTTCTGGCCATAGTTTTCCTGGGCCTGTGTCAGTTCAGTGCGTCC
AGATGCAGCTGGGTTGAGACTCAAAGCCCTGAAGATACCGCAAGGGGAGTCCTCCTGTGTCCACTTGCTCTGACCTGCACAGCAGGTCG
GAGTGCCCGCCGCTCCCCGGCTGCCAGCTGTTTACCTGGCCGGCCTTGCAAAGGCTCTTCAGCGCCAGGCCCCACCCGGTGAATCACTG
AAACTGATCCCCGTGGCCACTGTGAAGTACGGGACTGGAGGTCTCCGGAGGAGATGGCAGCTCTAATAAAAACAGGCTGCCAAACCGCA
GAAACTGTATAAGCCTCTGTTTCTCATCCTCGCTACCACCCCTCCGTAGTCCCTGTTTTACAGTTGAGGAAACCGTGGCCCAGGGAAGG
TCGGGAACTTGTCCAAAGGCACAGTCATAGAACTAGGGCTTACACCAGCTGTGTCTAATAGCATGCGTGCCCCCTCTGCTCTGCTTTTA
TTTGCTTCTTGGAGTGCTGTTTAGAGAAATTGATGGTTCTTTTGTTGTGACTAGGGGTCCGCACTATGGGTGACCTCCCTGCTCCCTTT
GGCCTCAGGCCCTTATCTTAGGCGATGGCCAAAAGAACAGCTTTACATTCAGTAAACTGTTTTAACTGAACCTCACCACAGGTTTACGG
CTGGTGCTCGTTTCATAATGGATAAACAATGGGTGTGGCTCAGTGTGGGGAAGTTGGTGGATGGGGAGAGGGCAGCAGAGTAACCAGCT
TTTAGTGGAGACCTGGATTTGAGTGCCCTGAGTTAGGTGAGCCTTTGTACCTCTTTCTTCACTTCTTGGAGCAGAACATTAGTTTCTCC
AGATCCCACATGTCATAAAGTTCTGTGATCCTGCAGTTTGAACATCCTGGCATTCAGCTCAGCCCACTCTGCAGAAATCTATACTAAAC
ATGTAGACAGTTTAGGAAGAGGGATTGTTTATTGTGTTGAATGTTATAGAATAATAGCTACTTAAATTTTGGAGCACTTGCCATTCATC
TTCCAAGTGATTTCTACGTGTTGACTCATGTAACACAGGCAACCACCCTATAATAAGTACTATGTTTACACACCCACTCTACAGATGAG
GAAACTGAGGCTCAGAGGAATTAAGTAATCTGCCAAAGATCCCTTGGCAAGTAAGTGGCTAAGGGGGGATTGGAACCCAGGGAGAGAGC
ATAATCCTAGGAGGAGATTCTGCCAAGGTACAGCCCTCCACCATGGAAGGGCAGCAGCCTCCTGTGAGCTGACTCACTTGAGTTATTCC
ACACCATCCCAGTGGAGAAGTTGGATTATCCCTATTGTGATAGAGGAAGAAAGGTGAAGGAATTTGCCAGAAATTATAAATGGTGACAT
TCGCATTGAACACATGTCCACTGACCCCAAATACTGGGTCATTTTATATCTTTCCCTCCCTTCTTTTTTTTTTTTTTAGACTAGTCTCA
CTCTGTCACCCAGGCTGGAGTGCCATGGCACGATCTTGCCTCACTGCAACCTCCACCTCCCGGGTTCAAGCAATTCTTCTGCCTCAGCC
TTCCAAGTAGCTGGGATTACAGACACCTGCCACCAGGCCACACGAATTTTTTGGATTTTTAGTAGAAATGGGGTTTCACCATGTTGGCC
AGGCTGGTCTTGAACTCCTCACCTCACGTAATCCACCTGCCTTCACCTCCCAAAGTGTTGGGATTACAGCCGTGAGCCACGCCCACCCA
CCCCCTTCACACCCCACCTTGTTTTTTTCTTTTTAGTAGAGATGGGGTTTTACCGTGTTGGCCAGGCTGGTCTTCAACTCCTGATCTCA
AGTGATCCCCTTGCCTCGGCCTCCCAAAGTGCTGGGATTACTCGTGTGAGCCACCACGCTTGGCCTCTTCCCTCCCTTCTTGCTAACTT
TGAAGCTCAGCTCCTTGTCCAAGAGCCTTTCTGTCCAGGTGTTTTCATGGTGCCTGTGCCTTCTGCTGGTTTGGAGTGAGCGGTTTCCA
CAGCATCCTCCCATCAGCCATGCATTCCTCAGGGTCACGCCCAACATGCTCTTCATATCTGCTCCCTGCCACTTGCCCTTCAAACCAGG
AATTCACTGCACCTAGCCTGATCCATTGACCTGGGCCCAGTATAGGCCCTCCCATGTTGGGAGGCCATGGGGTGTTAAGGTTAATGCTG
CGCCACACATGACTGTGATGAGTCCCTTTCTCTCTCCTGAAGAACGTGTTTCCATCCGCGACAGGGGCAGTTAAGCCTGGCTTCTGTTG
GGCGCTGCTGCTTGGGTGGGTCCAGTCTGCCCCATGTGGGGTCTGCATTCTATGCCAAGTGTTGCCACCTTCTGCCAGGTACACCTCTT
CAGTATGGAAGCACTTCCCTGTGCTCAGCCTCTCCCCGACTGTGCACATTTCCTTCTCTTCTGCTGGGGAAATGGAAAACAGCTGACCA
CTGATTCCTTGAAATCACCTACTCAGACACTCCAGGGTCTGTGTGGGATTAAGTCGTCCGCAGTCTTTCCCTCCCTTAATCTCTTCTAA
CCTTCCTCCAAACTCCCCTTTCCCACCCCATTTTAATCATCCCCTTTGCTCTCGCCTGGACATCTCCACGGCCTCCAGTGCCTCTCTAA
GTGAAGCGTTTGGGGCCACACACATGGCTGTCTCTTCTCTCCACGCCCACATGCAGAATCCAGCAGCCCCCTGCCCTCACACCTGCTTT
TCAGTAGTGGCTGGGACTGCCAGATGAATAGGCTTGAGGCAGCATACGAGTTTGCTATACACTTGCTCAAAACATTAGTGGACTCAAAA
CCAAAAAGACAACCCAGTTTCAGAATCGGCAGAAGACTTCAACACACATTTCTCCAGAGAAGTTATATAAATGGCCAATAAACACATGA
AAAGATCCTCAGTAGCCATGGTTGTTACCGAGATGCAAATTAAAACCATGAGATACCCCTTCATACCTACTAGGATGGCTGTAATTAAA
CACCACCACCACCAACAGCAAAAAAAAAAAAAAAAAAAAAAAGGCAGAAAATAGCAAGTGTTGGTGAGGATGTAGAGAAATTGGAACCC
TTGTACATTGCTGGTAGGAATGTAAGATCATACAGTCTCTGTGCAAGACAGTTTGGTGGTTCCTCAAAAAGTCAAGTATAGAATTACAT
ATCACCCAGCAATTCCACTCCTAGGTGTATATCCAAGAGAGTTGAAGGCGTATGTCCACAAAAAGCTTTTACAGGCATGTGCACTACTC
ACAACAGCCAGAACGTGGCAACAACCCACATGTCCAATGTCTGTCCGCGGAAGAATAGCAAACAGTATTCAACTGTAAAATGGAATGAG
ACATTGATACATGTTACAACATGGATGAACCTCAAAAATGCTGTTCTAGGTGACAGAAGCTAGGCACAAAAAGTCACATATTGTCTGAT
TCCATTTTTATCAATTTTCAAGAATAAGCAAAATTATATAGACAGAAAGCAGATTAATCCTTGCTCGGGGCCATGGGGAGTAGCTGCTT
AATGGGTACAAAGTTGTTGTTGGTTTTTTTTTTTTTTTTTTTGAGACCGAGTTTCCCTCTTGTTTCCCAGGCTCGACTGCTGGAGTGCA
CTGGTGCAATCTCAGCTCACTGCAACCTCCACCTCCTGTGTTCAAGTGATTCTCCTGCCTCAGCCTCCTGAGCGGTTAGGGTTACAGTC
ATGTGCCATCACACCCGGCTAATTTTCTATTTTTGGTAGACATGGGGTTTCACCATGTTGGCCAGGCTGGTCTGAAACTCCTGACTTCA
GGTGATCCACCCACCTCGGCCTCCCAAAGTGTTGGGATTACAGCTGTGAGCCACCACACCTGGCCAATGAGTACAAAGTTTTATTTGAG
GGTGATGAAAATGTTTTGCAACCAGATACACATGGTGATTATACCACATTGTGAATGTACTAGACAAACGTCACTGATTTTGTACACTT
TAAAATGGTTAATATTACTTTATGCAGATTTTACCTCCATTAAAACAGNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNCCTGTAATCCCAGCACTTTGGGTAGGCCGAGGCGGGCGGCAGTATCACCTGAAGTCGGGAGTTTGAG
ACCAGCCTGACCAACATGGTAGAAATCCTTTCCCTACTAAAAATACGAAATTAGCCAGGCGTGGTGGCACGTGCCTGTAATCCCAGCTA
CTTGGGAGGCTGAGGCAGGAGAATTGCTTGAATCTGGGAGGCAGAGGTTGTGGTGAGCCGAGATTGCATCATTGCATTTCAGCCTGGGC
AACAAGAGCGAAACTCCATCTCAAAAAAAAAAAAAAAAAAAAAAAAGAGGATATGTGGACACTATGTCTGGCACTTAAACTGGTGGATT
CTATAGAGATGGGATGGGTTCATTAGCCCCATTCTACAGATGAGAAAGTAGAGGCTCCATTACAGCACACAGTGTAGAAGTGGAACCAG
GATTGGTCTGACTCTAGAATGCTGCTTTTGATCACTTTTCTATGTTCCTCTTCCTCTACAGTCGACAGGTTTTGTTTTGTTTCGCTTTG
CTTCAGATTCTTGGCTCATGCCAAGTGCTGTGTGCCATGGGTACATCACCTGACCCTGAAGAGCCCAGATCTATGTCTTTTCATGCAAC
CTTGCCCTGTGCTTTCCACATATTCACATCCTTCACAGTCAATCCCACCCTGGCCACTAGGCCAATAGGGACTCCCCACATCCCACTTA
CAGGGGGGCAAATATTTATAGCCAAAAGAAAACAGAAGATGTGGGTTTGAGTCACAGTTCCACTATGTGGTCATGGATGAGTGCCTTCA
ACCCTCAGTACCTCAGTTTCCCTAAATGTGCAGTGGGAGAGCATGAGAACACTTATCTGATGTCTTCCCTGGGGTTTTGTGAGGATTAA
ACACACCATGTAGGGGCAAGTGCTTTGAGAATGGAAAAGCACTGTGCAGACATGAGTGATGTGCATGATGGTTATTGTTAGATGTTCTC
TCCTGGAATCAAGTTCAAGCTTCTTGCATACTCAGCAGAGAACAGACCCAAACTCCCGAATCTAGTGCTGTGGCATGTGGTTGGTGCTC
AATAATGATTTGTTGTGTGAACAACTGCATGAATGAGTAGTAGGTCAATGACCTCAATGTCGGCCTGGTCAGGATTTAGATTTCTTGTC
TTTGTGTGTGCTCAGCCCAGGACTGAATGCACAGCAGGCTGTCAGGAAGTAGTCATGGATTGATGTGTCCGTATTCGCCATAACCAAGC
AGGAGATAGATGATTTCGTTCTGGTCTCTGGATTTTGGCTGCATTTTCTCTCATAGTAGGAATTATTCCCGGAAGATCCAAATGCTCAG
CTTTGTCCTCCTCTCATCCTCGTGAGGCGTGTGATCCAGGTGAGAAGCAGGGAGAACTGCCCTTCCTGGTGGGAAGTTGTGGGCCATCA
CCTTAAGCTTGGCTTGTAGTGGTGGGAGACCTGTGTTCCTTCCTTCCTGAGCCCCACTTCCTCTTGATTTTATTGTATTTTTAGCAGCA
CTGACCTGAAGCTTTCTGGGTTGCATGTTAGAGGCAGCACTTCTTGCACAACCTAGGGGAGCGTGAGTATCGTTTCGGTTCTGATAGCA
GGATGGATTGTTATGTATCCTTTCTGATCCAGGCTACCAAAACCTTTAGATGTTAGGATGGATTATCTCACCTTGCACAAGACAGTGAG
TGCTAGAGTTTAAGTTTTCTCCTCTGCAAAATAAGGAGTGGCAGTGACACTTACAGCATCTCTCAAGCCCACTTTGGCCCAAAACTTCC
ATGACTGTGGCAGTCTACAGTAAATGTGTTTGATAGCCCCTAGTTACAACTCTCTTCTGTCTCTTCCCCTTTCTGCCTGCGCTACCATC
TAATCCATCCACGCAACCACCCATCCACCCACCCACATTTCCTAGCCAGCTCTCTGGACCAGGCCTTGTGTTAAGTCCTGAAGACACAC
AAAAGGCTAAAACAGCCCCTGGTCTTAAGGACTCACAGATCATGAGGACGCGATAGATACTGGAATAGAAGGCAAAAGTCAAATGTCAT
GAGTGCTGGGATTCAGAAAGCTGCAGGCTGCTCTGGTAGCAATAAAGGAAACCAGCTCTCCCTTGACATCCCTTTCATGAGGGTTATAG
CCTGGATCCTGTGATGACCCAGGACAGACTGGTCTTGATCCAGGGCAACCATATCAGTGACCTCAAGAGGCCCCTTTTAGCCACAGTAA
TCTATGATGTGGAGTTCTCAATTGCTGGCTTTCCTGGAGAAAGAGAGAAGGAAGAAATAGAGGGGCCGGTCAGCCCTCCTGTACATTTA
GTTTGCGGTCTCAAACCTCTGTTTCCTGGAGCTGCAGGCTGTATGTGTGGGAGGTGCACAGCAGTGGCTTCGCCCCCCCAACGCGCCTG
CAGCCCGCTCTGGGGACAGCCGTCTTTTGGCAGAGTCTGCCTCTGCAGTGGTCGCAGCTGGCCCAGGCAGACCTCCTCTCTGTCCCGTT
AGTCAGCGTCTGGTGGCCAAGGCCAGAGTGGAGCCTGTGTTTCCGACAGGCCAGCTGCCCCTCAGACTCAGGCAGCGCCGTGATTCTGA
GGGTGATGCTGGCGTTCGCCCCTCAACCCTGTCCCCCACAAGCAGATTACGCACAAGGCAGCATTTCTAATAAGCAGCCTCTGCTTGCC
CCGTAGCAGATGAAACACTGCGTGTAAGAGTGGGTAATTTTGGGTTTTCCTGATGAAAGTAGTTGACATTTGTGCTGAGACCTCATGCG
CTCTGCAGCGCTGCCTTTCATCTCTGCTCTCAAAGCCCTGCTCATCTCTGCCATGACTCGGGGGCCGCCTGCGCCACCGGAATTCAGTT
CGGCCAGTGGTGAAATATAGAGGAAATGGTGGAATGACCCCTGAAGGCACTGGATGGGGGAGTGGAGACAAGTCCTGACTTTCCTGGTG
GAGGGGATGTTCCTGGGGGTGTGGAACTGTGTATCGGCCCATGCAGCACAGCCAGGGAAAGAGTCTGCATCTTGGAGCTTGGTATGGCT
GCCTAGATTGGAAAGGCAGCTCCTCCACGGACTTGCTCCGTGAATGCAGGGCATCCTTTCTCTTCCCTGATCTGTGTCCTCATTTGTCA
GTGGGAATAATAGAATAACATTACCACCAACGACGATGGCTTTCACAACTGGAACCACTAGGCCCTCAGGAATGTTTACTGGCTTCTAG
TCTCATTGATAAACTGAAGCCATTCTGCCATAGCTCTAGAGAACTGGCACTAGGGGCTTGCATGATTCATCTGAGTTCCCCAGACAATG
CTGGCACATAGTAGGCCCTCCATAAAGTAAGAAGGGACACTTTGCTCCTGACATCCATACAGCTCCTCTAGCTTTGCAGACAAACCCCT
AGAGAGAAAGAATGTGAAGGAACAAAGGGTCAGGTTAGGTTGTGCAACCTTTCTGATATGCATTTGTAATGCAGTGAGCAAATGCACAA
GCCTTGAGTCGCTGCATTCTTTTCTAACCACCCAAGAGATAAGTTGAACCCCCACTCGTAGCAACCTGGCCACTTGTCTGTTTAGTCAG
CCCTGGAATACCTCCACTGACGGGGCACTCACTACCACTGAAAGATGGTGTGCAGAATTCTAATGTTCTTCCTCTCGCGGGGTCGTGGG
GGCTTTCTTCCAGTTCTCTGGGTTCTGATCGTTGGGGCTGTCCTGACACCCTTCTTCAGATGACCGCTGTCAGGTTGGTGAAGGCAGTG
CGCGTTGGGCCTTCCCTCCCTAGAGATGTGATTCCACGTCGGGTACCAGGAGCCTGGCCACCCCTCAGCTTCTGCTAACCAGGGAGCAG
TGAACCCCGCTTTACTTTTGTTGGCTTGTTTATTTTCTAATGTCACCTGCCTCTGCACTTTAAGTAGTATTTGGGCTGCTATGGAGTGT
GCACATTCTAACACAGCACCTTGTCAGCTGGCTGAGAAGCTGAAAATTCTCGGCATTCCTCCTTGATGTCAATTACTTCAAGGAGCAGG
AAGGTGACTTCTTTTAACAAGTGGAAAGGATTCAGGTAACAAAGCCATTTCCTCTTCTTAACCCTCTACTGTCCAAAGTTATTGACTTA
GCGACACAGTCTGTTTATCTGGGCTTCACGAGCACGGCTTTCGACACTGAAGGCTTCTCCCCACTTTTGTGCAATGTTATATATTTTTT
CTCACTCTTCATCCAGTCTGAGTCCCGTCTGCCCCCTCCCCTGAGCAGTCACCATCCTGCTACCCACTCCCCCTCTATTGGCCACTGTG
GCATAGGGTGGTGGAAAGGACGCTGGACTGAGAGCCCAGAGATCAGATGTCTGACCTTCCCTCTGCCTGGAAGAGCTGCCTGGCCTCTG
ATCACGGTCCCTACCTTCGTTTTCTGGCCTCACTCACTGTGGCCCACCATCCACCAGTCCAGGTGTCCTGGGCACTCGGGGAACGTCAG
AACTCGAAGGAAACCCTCCAAGCCCTCCCATGCAGGCCCACCATTCCACTGTCAGGTTTTCTTCCCTTTCCTTCTCCTCTACATTTTGT
GCCAGCAGTTTGGGGGTCTCCTGAATCTTATCCCTTCGCAGCGGTAGGCAGGTCCAATTTCTGCCCAGTCACTGAACATGAAGAAGCAT
CTATACCCCGAGTTAAATCCAAAACACTTGGCCTGACACACCTGCCCTTCAACTCTCCCCCAGCCTCTCCTCTTGTCCCCAACAGTGGT
TCATCTTCTAGCCACACAGAAATTGAGGGGGTCCTGTGTTATCTGTGTTCCTGCAGACAGAGCTTCCCTAGGACAGCAAAAGTCATCCT
AACATAAAAATATGCATGAATAAACTCACTGCCAACATTCAGCCCACCCACATCCAATCATAATCAGTCATTTACTGATCCCCAGACGT
TGTAAACATCTGTCAGTCCACTGCAAGAACAGCAGAAACTCCTAGCTGACCTCCATTTGGCCTACTCACTCAATTAAGTCATCTTACTC
ATTCGCTTTGTAAAAGCAAACTGAGTGAGGCCCCAGGCTCATTGAAGACTCTGTGCCTTACCCAGAACCCCAACTTTCCAGATTCAATA
TTTTTTCTATTTCTTAGCTGTGTTACCTTCCCCAACTTAATTAACCTCTCTCTCCCTCCATGTTCCATCTGTAAATGAGACTAATACTA
GTACCCACCTTCTAAACTCATTATGAGCATTAAATGAATTAGTACGTTTAAAGGCTTAGAACAGTGTTTTATGATACGATAAACACTCA
ATAAATGTTAGCTATTGATATTGGTGTGCCCAGAAGGCTTGTTACTACTAGTTGATTTATGTCCTTCCCAAAAGTTGTTGTGTTGGTAA
TTAAGTACGACATAAACTAATCAAAATTGACTTTATAAAGAGTTGTTTTATGAAAAGTTTAGTACTTTGGATAGAATGGATAATATTGA
TTTGCTAAAAAAAAATTGTCACCAAATAACTTAAAGCCAAAACAGCTGTAAAAAATTGTAAAACCATAGTTTGTATTCCATATGCTTTA
CAGGTGATTAACTTTGCCACTGCTTTAAAGAAACTGAAACCAGCAATTGTAGATGTTGACTTCTGAGTGTGTATTTTGCATCGAAGATC
ATTTGCATCTCTAGTCACCGTGCCAAAAAAAGATCCTACCCTTTGATTGGTTAATAAATATCCAACTATAATAAAATATTTAAGTGTTG
GTCCTTAATGATTGCCTACATTTTCAATGAAATGACAGTGGCACAACTGAGGAACAGGCGTTTTTCTCTCCAAGGCAGATCAGCTGCCA
CAAATTGCACGTTTTGTACCAGCTGCAGGGTTTCCTGGAATCAGCAAACGCCTGTGAGAGGCTGCATGAACCACACACGCTTACTGACA
CCTCCTGTGTGCCGAGCGCTGAGTAGTGCTGAGGATATAGCAATTGGGGAGCACTACCACAATCAGCGTTGTTAACCGTCGAGAAGAGA
AAGGAAAGATGATGTCTCTCTATGCAAATCAAGGGAAAGTGATTTAAAAATGTTAAAAAAAAAAACTTAACAGAGCTCTTCGAAAGGTG
TTTTGCAAAATGATTCCTTGGATTAATGATGAAGGAGTGGAGTGAAGGAAGTTTATATTTTGTGAGAAAACGTGTGCTAAGGAACCCCT
CGACATTAGATTCATTCTGTACACAACTCAGCAAATTAGATCATAGTTTAAAATGATAATTTCAGGGGCCGGGCACAGTGGCTCCTGCC
TCTAATCCCAACACTTTGGGAGGCCGAGGAGGGAGGATCATTTGAGGCCAGGAGTTTGAGACCAGTCTGGCCAACATATCGAAACCCCA
TCTCTATTAAAAAATACAGAAAATTAGCTGGGCTTTGTGGTGCATGCCTATCTTCCTCTCTACTTGGGAGGCTGTGCATGAGAATCANN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNACTGCAACCTCTGCCTCCTGGGTTCAAGCAATTTACC
TGCCTCTGCCTCCCGAGTAGCTAGGACTACAAGCATGTGCCACCACGCCCGGTTAATTTTTGTATTTTTAGTAGAGACGGTGTTTCACC
ATATTGGTCAGACTGGTCTTGAACTCTTGACCTCGTGATCTGCCCGCCTTGGCCTCCCAAAATGCAGGGATTACAGGTCTGAGCCACTG
CACCTGGCCAGTTCAGGTTTCAAAGTATAACATCAATCTTATTTTATGTATAGTTTATGTGTAATTATAAATTGTATACTAATAAAGTC
ATTTTTATAATTCTTTATTTCTTTTTCCAAGATACCCACCCCCCCCCCACCAAGGAGGGCTAGGACAAGTGACAAAAGTCACCCCAGAT
CACAGCCAAATGGGTAGAGCTAGGAATCCGAAGTAGGTGATGAAGCTTGATTTTTCAAGATGCTGCAACCCCTCTCCCCCATATGCTAC
ATTAAATATCTGCACCTATTTTAAGTGCGTTCTTTTCTGGGGTAATGAGGTTCTCTTCTGCCTCAGTTTTCCATGGCTGCAGAGCTCCT
GCTGTTCCTTCTGCCTAGTTCACTCTTCTTTGCCTTGACTGCACCTCTCTGAGCTACCTTCCTACCTCCATCCTTGCATTCATTAGCGG
TTTCTTTATGTGTCCAGCTCACTGTGGGACCCAGGACTCCTTGAAGAGGGAGATCTTGCTCATCCATCTGTGTCCCCCACTCTACTGCC
AACACAGTGGTAGGAAGGGAAGGCGGCAGGCTTAGGCGAGGTCCTGCAGGCTGGACAGGATCTGTCACCATTACTTAGCCCAGGCCCAT
CTCTTTACTTCCAGACCAACTGGTGTCACCATAATGCCTTCTGGCAATTTGAAGATTCTGGCAACCTCTTACCGGATCCAGCTGGCAGC
ATAATCCCTGTGCATTAGCCACTTTCAGAGGTATCTCCGCCCCCAAAAGAGTCACGGGAGTCCCAGACATGAAGGAGCATTAGTGGCCA
TCTCCCCACCTTGCTGTATCAGCAAAACCGCTAAGCCCTGACCCATATTTTGGGCAGTGCTCACCCTATCTCATTTTATCTTACTGTAT
ATCTCGTTTGTATCATTTTATTAGGTATCTTATTTTATCTCATTATATGCTAATCTCATTTGCTGGGTAGACCAGTCACCAGGGCTGGC
ACAAGTGACTGGAATTGCCCAAGGTCACAGACAAGTGACCTTTACAGATAGGACTCCGAAGCAGGTGGCGGAGTCACCGTTGAGTTCAT
CATAGCGATGCTGGCTGCCTCAAAGTACTGTTTTAGTTAATGCTCACAAGCAACTCTGCGAAGCAGACGGGCCATTCATTTGTGCTTCA
CAGTGTGGGAAACTGAGTCACAGCAGGTTCATGTCACACAGCTGATAAATGACAAAGCCAGATGTGAAGCCTGTGACTTCCATTCCAAC
ATTCATCTTTGATGAAGCGTTCAGGTTGGGGCCTGGTGTGGGACATGCTCAGAGGAGGACGAGGAGGATGATTTGGGGGAGGCCTCAGA
AGGCGTTCCTGGTCTGAGTGTGCTGCTCTCACTGCCTTGGTTTGGGTCCCCGCTTCTGAGCAGGATCTTCTATTGGCAGACGGTTCAAT
CCAGTGGGTGCCATGAAGGAAGATGTCTCCCCAACAGCATCACACTCTCACGGACTGTTCACATCACATTAAAAGCGCTGAGACTCTGG
CTTTTGGTGAAAACGGCTATTTTGTCTTGTTCCACGGGAGCTGGGAAGAGGAGACTAAACACAGGAGCCTCCCCCTAACCCACGGGCAC
ATCGAGAAGGAGGAAGGCAGCCCATGCTGGGCAGCAAAAGGGAGAGAAGGCGAGGGCCAGAGCCCTGGGAAGTGTCAGATCCTGGTGTG
CCGCGCTTCTCCCAGGTTGTATAATCAGCTGTCACTTCAGCATTTCCCTGCATCAGCCTTCCCAAGTACAGATTGAACATTAAACAAAC
AGTGACGGCTTTATAAATATTTAACTCCCACTGTTTAGGAATTGCTCGGGGCAGGAGACAAAATAAGAGTTCCTGGCTGTCTGCTCCAT
GCCCCCTCTGGCATGGTGTTGGAGGCCGCCGCTCAGAGATTTGCCGAGTCGTGGGCAGCAGCTATTCTCTTCTTCCTTTCCAGGGGGAT
GTGTTTGGGGACACATGTCTGTGTGTTCATGGAGCTGTTAGGGCCTCTTCTAGAAGCCACCAGTTTGCGTTTTCATACAGTGGCTGGGA
TCCATCATTCCTGGTGTTATTCCTTCACATCAGAGCAAACTTCTTCCTTTCTTTTTTATATATAGCACCTTTATTGAGATATAATTCAC
ATATAATTCACCTGAAACTCACCCATTTAAGGTATACAACACAATTTTTTTTGTTTGTTTGTTTCTTTGAGACAGAGTCTTGCTCTGTG
ACCCAGGCTGGAGTGCAGTGGTGCCATCTTGGCTCACTGCAACCTCCGCCTCCCCGGTTCAGCCTATTCTGCTTCAGCCTCCCCAGTAG
CTGAGACTACAGGCACACACCACCATGTCTGGCTAACTTTTGTATTTTTTTTTTTTTTTTTTTTGAGACAGAGTCTCACTCTGTTGCCC
AGGCTGGAGTGCAGTGGCTCGATGTCGGCTCACTGTAAGCTCCGCCTCCCGTGTTCATGCCATTCTCCTGCCTCAGCCTCCTGAGTAGC
TGGGACTACAGGCGCCCACCACCACGCCCGACTAATTTTTTGTATTTTTAGTCGAGATGGGCTTTCGCCGTGTTAGCCAGGATGGTCTT
GATCTCCTGACCTCGTGATCCGCCCACCTCGCCCTCCCAAACTCCTGGGATTACAGGTGTGAACCACCGCACCAGACCCCAACTTTTGT
ATTTTTAGTAGAGATGGGGTTTTGCCACGTTCCCCAGACTGGTCTTGAACTCCTGGCCTTAAGCGATCCACCCACCTCGGCCTCCCTTA
GTGCTCGCATTATAGGCAGGAGCCACGGTGCTCGGCCAACACAATTGTTCTTAATATATTCATGGAGTTGTGCAACCATCACCACAATC
AATTTGAGAGCATTTTGATCACCCCAAAAAGAAATCGTTTTTATTACCACTCACCTCTTTATTCCCACAATTCTCTGCGTGCTAAGAAA
TTGTTGAGCTACTTGTACTACAAATATACCTATCCTGGAATGGTCTTTTGTGCCTGGCTTCTTTCACTTGGTGTAATGTTTTCAAGGTT
TATCTGTGTTGTAGCATAGATCAGCATGTCATTTCTCCTTAGGGCTGAATACTGTTCCCTAGCACAAGTATATTGCATTTTTAAAAATC
TACTCATCTCTTGATGAACATTTCGGGACAGCAAAGCTCTAGTTCAAAAATCAAGATCAAAAATCAAAAAAATATAGTTGAAAAAATCA
AAAAAATCTAGTTGAAAAATCCAGCATCATGTGATTAAGGCCAAACTCAACTTCACAAAAGAGAGCCTCTACTATTTTGGGAAAGATCT
AGAAGGTTATCTCTAACCATATGGTGGATGTTACAATGTCTTCAGTGGACACGCCATTCAGCTTTTATTCACCACTTACCATATGCTCG
CCACTATGGTAGCATTCATTAATTAATTTATTGAAAATAATTATTCACATTATATAGTCTTGTGAAAAGATTTATCAGAGGCTGTTCTG
TGAATTTTAGAGGTTTTTTGTTTTTGTTTTTTTTTGAAATGGAGTTTTACTTTTGTTGCCCACGCTGGAGTGCAGTCGTGCCATCTCGG
CTCACCGCAACCTCTGCCTCCTGGGTTCAACCCATTCTCCTGCCTCACCCTCCCAACTACCTGCCACTATAGGCATACACCACCATGCC
TGGCTAATTTTGTATTTTTAGTAGAGATGGGGTTTCACCATGTTGGCCGGGCTGCTCTCGAACTCCTGACCTCAGGTGAGCCACCCACC
TCGCCCTCCCAAAGTCCTCGGATTACAGGCATGAGCCACCGTGCCCACGTGAGTTTTAGACTTTTTTCGCCTCTAAGAATGCGACCATG
AGTTTGTGTTCTCAGCTCGTTTCATTTGATTTCAGATGTCAGCTCTCTTTAGGAGAGAGGCGTGGAGAAAAGGTGGGACTCTCTGCAGT
CCTGTTCTTGTTCTGCCCACTTCCCTCCAGTCATGGTCTCTGGCCGGCTCTCTTCTCCCCAGAGTGGTTCTTTTCCTGCCCCTGGTGTC
CCTCTAAGACCCAGATGTTTCCAGCACCCATTTGAGAGGCTGATTCAGCCCAGGCAGTGTCTGGCCTAGGGAAGGTGACTGCAGGAAAT
GTTAAAAAATGGGGGCTGTTATCATGCCTGAGCATCGCACAGTCCCTCCGTTGTGACTAATTGTAGTGGTTGTTGTTGAAGGGCTGCCC
CAGGTCCCCCTCCCTGGACCGGTACACTCACCCCAGCTGCCGAGACGGCGGCTGCTAATGTCCACTGCTGCTAATGTCCACAGCACCAC
TTCTCTGGACATTAGCCTTCCTCCAACTGGAGCTGCTTCATCCGGGAACTTCCTCACCCTGCACCACCCCTCGCTGACTCAGCGGTGCA
AAGGGCTCCTCCTCCTTGTTTCCATGTGGGGTAAACTCCATGGTGCGGTTCATGTTCCACCCCATCCTGTGGATGGCCTGAGTTGGTCC
CCAGCCGAGACCACCTCCTTCCCTTGCCTGGCTCTGCCACCTTCCCTTCCTTTCTCCTGAGGTTGCTCCCTGTTAAATCACACAGACAG
ACATTCCCTCCTCAGGATCTGTCCCCGGGTGCCCACCCTCAGACACTATCCGGCAACACGTTCCCTTTCCTGAGTCCCCTCTGATGATG
TATCTGACTTGGAGATCCTTGGCCTGCTCCCTCTCTTTTCAGTTCTCTTCTGGAGAATAGAAGCTTCCTGGGCTGGCCTGTTGAGTGTT
TTGTTTGGTTGGTTTTGATTTCCAAGTCTTTTCAGGCTAATCGGACACGAGGAAAGGTCTGCATTGAATCACAGCCTTCCCACTGTTCC
CCCCTTCCTTCCCTCCTCCGTTCATTCCCCCGAACATCCTTTTCTCTGCCGACAATCACCTCACCAGAAAGGGGAAAAGGACGTGGGAA
GGCTGCATGGGAAACTCATGGAACTTGGTACCAAACAGCTCTGGGTTCAAGTCCGCGCCCTGTTGCTCAGGAGCCAGGCACTCAGTTGT
GCTGGCTTCTCTAACCCCCAACTTCTTCTATGAAAATGGGACTATTCGTCTCTACTCCTTGCATTATTCTCGGGATCGGAAATAATCTG
GCTCAATCATGTGGTGCAGTGACTAGCATCGACGCGACACCCTCTCCACACAGGCTGGAAAGACATCCAGAAGGAGTCCTGGGCTCCCT
CACCACGCAGGGGCTCAGCCTCCCCAGATAGTTGTCATCACCCATCCAGGCCCGTTTCTGGTCTCATTGCTTTTGTTCATGTACTTTCC
TCCTCGGTTGCTCTTCCTTTTCTCTCTGACCTGTCAAAAAGCCCCATTGTAAGCAACATTTAAATCCTACCTCTTCCATGAAGCCCTTT
TGGATGCCTCTTAGGGGGATGAGTTCTTTCCATCCTAGCACTGCTTGCTGCATGGTCCAGGATCTGTGGGGCCTGTCTTCTTTACCACT
TTGCTCACTTCATGAATGTGGGGGTGTATGAAGAGTGTGATGAAATTAGAGGCTATGTTCTGGAGTCAGACTGATCAGTACTGGGATTC
TGGCCATACCCCCTCCTTAGCTGGGTGACTTCAGGCAACTGACTGTACCATCCTGAAAGTCATCTGTAAGCCGTGCTGACCCCCCAACC
ACAGGACACTTGTGCCCGCTGCACGTGCTCTGTCAGCAGTGACTTCTCACTACCTGGCATCCTCTGAATTCACGAGCAGTTTGAAGAAT
GACCAAATACTGAGCAAAGCTCATAGGATTAAATACACACAGACCTCTGTAAGTTAACACCAAGCTAAGGGTTAAATACTTTACAAAAA
TTCTCAGTGCGGTTGGTGGGTGAGAACGGCTGTCGTCCCCTCTGGCTGCCTTGCCAAGCTGGTAGAGGGGCCATCTCCCACTACCTACT
GACAAAGATTCTTTGCTTGACCCAATTACCCAATTTAATCAGACACTTAAGTCTTTTCCTAGCCCCATCTGTGTACTTCCTGTAAAATC
TAGTTTTAGCAAAAAACTCTGCTGAGTCAGTTTGGTAAGGACCACCGGCCGTCAATATCTTCCATATCTGATAGATAGGGTTCCTCATC
TTCTACCACCCTCCAGATGATCTCTGGTCACCCTGGCCTGCCTTTAGCAAGAATCTTGATAGGTTGATTTAACCAGAATCCCCCTTACT
CCTGATGTTGCCTGTTAGTAATTTTCTATCCTCTGACCCCCCACCCTCCTTCTTGGCTGTGAATTTCCACTTGCTCAGGCTGTATTGAG
TTGAGCCCTATCTTTCTCCTACACTGCAAATTCCCATTGCCGTGGTCCCTATACCTATCAGGATGGTCCTGAATTGTCTTCTTTATCAT
CTGCTCTGCTCTCCCAGAGCTTAAGCCCAGGCAGCTGAGTTAAAAGTGTGACCAAATGGACCATCTTGTCCCTCAAAATTGGGACCTAA
GCTCGATTCCGAGATTTAATCATGGCAGAGGAGTTTACTCTGCTAACCACCATCACCCCACCCTATCCCCCAGAGGGGTGCGGTGAGTA
AGGGGGTGGCAACTGCTTGTTTCCAATCTCCCATCTCTCTCTGGCCCTCCTCTGTCTCTGGCCTCCCTTCTCCCTTTGAAAGGGACACA
GTGCTGCTCACAGCCTGTCCTGCTGCGGTGGCTGCAGAGAGGGGCTGACAATAAGCCTGTGTCCGAGGCAGCCCTCCAAATACAGCAGG
GCTGGGTTGACCTTTAAATAGATGGGCTCTGGGCCACTCAGTTCCTGGAAGTTTGAAGAAAGTCAGCCTTTCAAGTGATCCCACTTCCA
ACCTCAGGAAAGATCGCTGTGTTTGTAAAGGGTATAATTTACTGTTGCTTTTGCACTTCATGATATGCGCCTGCACAGCCAGTGGGAGC
CAGCATGCCACAATTAGGAGGGGAAAATCCTCAAGTATTTTAATATACCCCTACTTAGAAGCAAGATAAACATATGTATTGTACTTTGG
GACAGAAGCAAGGGGGGTTTAGCGGGTGGGAAAGGGAGAGTCCTGGCATTGCATGAAAGGTCTTTTTAAGCTGAACTCTGAGGGTTTTC
AGTTTTTCATTTTTCTGTTTTTTTTTTTTTTTTATTTGTAAGCAAAGCTGGCATGAGCAAATGATGCCAGGATCTACTTGGCTGTGTAA
AAGCTGTAGTCCCTCCTCAGACAGGCAGACTCCTAGAACCCTCCCTAAGCCATAAACTAGTACACCGTTTTGCCTCGCCCAATTAAAAA
TGATTCGGTGTGGCTTGTCATCACAGTTGACTTAGATGATTTACTTTCTTCCAGGAAATCTTCCTCCCAACTCTTGCTTTGCAATTAGG
CAAAAGAAAAATAAATGTTAATATGTTCTGTGCTCCCAGGGAAAATTTTTTTAAAAACAAAATAAAACACTTTGGGAGGCCGAGGCAGG
CGGATCACGAGGTCAGGAGATCGAGACCATCCTGGCTAACACGGTGAAACCCCGTCTCTACTGAAAATACAAAAAAAATTAGCCGGGCA
TGGTGGCGGGCGCCTGTAGTCCCAGCTACTCGGGAGGCTGAGGCAGGAGAATGGCGTGAACCCGGGAGGCGGAGCTTGCAGTGAGCCGA
GATCGCACCACTGGACTCCAGCCTGGGTGACAGAGCGAGACTCCGTCTCAAAAAAAAAAAAGAAGTGTCAAGGTCATGAGTGTCAGGGA
AAGACTGAGAAATGGTTCCAGACTGAAAGACACTAAAGAGATGTGAGAATTAAATGCAAAGCTTGATTCTGGACAATTGTCAAACCTTG
GATCGCTCGCAGCCTTAGATGTTAGTTACGTATCTGTGTCAGCTTCCTGATCTTCCTATACAACATGGTCCTAAGGGAGAATGTCTTTG
CTTTAGAAAAATACACATGGAAGTACTCTGTAGTGATACGGCTTCAGGTCCACTACTCCCTCTCACATGCTGTAGGGGAAAAGGATTTG
CTGTGTGTTCCCAACACGTTTGTGATTATTTCACAAATAACAATAATTAAAAAAAACCAACCTCTGGTGGGGCGGTGCGGAATTATTAT
TATTTTTTTTTGGGACAGAGTCTCACTCTGTTGCTCAGGTTGGAGGCCACTGGCATGATCTTACCTCACTACAACCCTCAACTCCTGAG
TTCAAGCGATTCTTGTGCCTCAGCCTCCCAATTAGCTGGGATTACAGGCATGCGCCACCAGGTCTGGCTAATTTTTTTGCATTTTTAGT
AGAGACGAACTTTTGCCATGTTGGCCAGGCTGGTCTCAAACTCCTGACCTCAGGTCATCCAACCACCTCGGCCTCCCAAAGTGCTGAGA
TTAGAGGCGTGAGTCATCATGCCTGGCAGTAATCTTTTCAGTGGAGAATAAAATAAACCCGGGTTTGAATTCTGACCCTTTAAATTTAC
TCAGGGTTTCACTTCAGCCAGGGAACATAACCTTTCTGTGCCTCAGTTTCCTCATTTTTCAAAACAAACATGGTGTGTGTGTGCATCTC
CATCTATGAGTTGTTGTGAATAGGAATGTAGGGGCCCCAGCAGGGTGTGTAAAGGTACCAGTTCCTGGGGCGTCAAAGGTTTTTCTTTC
CCTTTGTACCCCCTCACTGACATGCCCACGTCTGTCTGTGTACAGCAGTGGGTGGATTTACAGACTCCGCTGAGAACCCCCTTATAGCA
TATTTTCAGGTTCGTGATGATATCATGGGCTGTGACCCTGGGAGTTTAATTGGAATGAATTTCAAGGTTTTGTTAAACTTGGCTAGAAT
CTTCCTATATCTCCAGCAAGATATTGATGGGGTTTTTGTTGTTGCTGTTGTTTTTGAGACAGGGTCTTGCTCTGTCACCCAGGCTGGAG
TTCAGTGGTGCCGTCACTGCAGCCTCAACCTCCCAGGCTCAAGGGATCCTCCCCTTCAGCGTCCCTAGTAGCTGGGACTACAGGTGCGC
TCCACCACACCCAGCTAGTTTTTGAATTTTTTTGTACTGATGGGGTTTCACTATGTTGCCCACGCTGGTCTCAAACTCCTGGGCTCAAG
CCATCTGCCTACCTTAGCCTCATAAAGTGCTGGAATTCCACCTGTGAGCCACTGCACCTGGCCAAGAACTTGGTGTTTCTTATTTCCAA
GCTTATCTCCTCTGTTATTTGCTCCCAGGGAGACCCCTTTTCCATTTATGTTTCCAGGGTGCTGGGGGGAAGTGATTGCCCTCATCTCT
TTTGCCCAGCCACCAAACCCAGTGCAGACTCTGCTGAATGCAGGCCCCATCTCTGTATTTTACTTATTCCATTTCCTGTCTTGAAGGTG
GGGGCACTATGTTTATCTCTAGGACCCTGACTGGCTCATCATTGAAAGCTTATTTTGTGCAAAGCATTGTTCTGAGCACTGTGTGTGTA
TGATTCTACTTCATCCTCTGTAACCCTAGCAGTCCGGTCAAATAGTATCATTTTATAGACAAGGCAACTGAGGCACAGAGCAGCTCAGC
AGCTTGCCCAGATCACACAGCTGGAAAGTGGTAGAGCTGGAATCTGGATGGGTGCTCGCAGCCTGGCCTCAGAGTCGGTGCTAACCACT
GAGCGGCACTGCCACCCAACGGATGGGAGCCTGCAGGATGAGGCCATGGGAATTGATTTCACTCAGCGCGAGCCAAGCAGAACTCCCGT
CCAGACCTCTGTCCACCACCCTAATAAAGCCAGTGTTTACACAGGCTGGAGGTGCTCATCAGATGTTTTTGCCAATCCCCAAATACGGC
CACGGCCAGAAAGTGAGAGGGGTGCCTATTATGTAACGCCCTGCGGCTGGTCCCCAGACAATGCCACTTCCTCGCTTGGCGCTGTGTGA
CTGCATAACAACGGCTTACTCATTTCCTCTGCAAAACTTCACAGGGCCTCTTTGCCCCATTTCCTCCTCCCACCCCATCCTCAGGCTTC
CTCTGTTGAATTTTCTCCAGATTCCTGGGAACACCACCATGGAGGGCCTGGCCTTTGGCGGTGGACAGTCTGTGGCTGGGTGGGAACGT
CAGGAGTGGGGCCTGCCTCTTTGCTGGGAATGCCGTGCACTCCCAGCCCACTTGCCCCATGCCAGCACTCGAGGCAGGTGAGGCCCAGC
CATGGGTATAAGGTGCTCCCAGGCCGGCATTAGCCTCAAGGAGGCAGCGGTTCCATGTTTCTGCTCCTCTCCCATTACTAACGGGTGTC
TGCTTTAGGTTTGATCTCCCTCCCTTCTGCCCCTCTCCTGGTAAAGGGATGAATAATGAAGGCCTTCCTTGGGAAGTCTGAGATGGATT
ACCCCAAGGAGGCTCGAACATGGGAGGACATGCGAGAACTATTGTCACATGAGTTTTCTTTCTATCCTGTCTGCAGGACTTCGCAGGTG
AGCAGTAACGGAAATCAGCCCCGTTTATTCCTCCCAGCACCTGCCTTATCCAACTCCCCACGCTGTGGCTGAGTCCCAGCCTGCTATGG
AAGCATCACTGGACTCCCATTGAACTCTGTGCAGATTCGCTGTTCGTAGACATGGTACCTGATGGACACCAAGCTACGTACAGCTTCAA
GGCCCCTTCTTTCTTTCCTAATAAAAAATAAAAAATAAAAACATAATACACGTTATATTTCTATCTTATAATACACATTTCTTATTTTT
ATCTCAATAAGCTGATAGATATATACTAGATATACACATCTCTCACTGTACATACATACACAAACACACACACACGTACATGCATGTAT
ATGCGTATCTATCTCCAATGCATGTGGAGTGTTGTGCCACGTCATCCTCACATTTAATTTTTCTGTTCTATTTATCATTCTTAAGGATT
GTCAGGCATTAGGGATTTATCTATAGCAATTTGGATCTTAGCAGTTCTTTTCATGGGTTATCTTATTTTTTCCTTCCAACAACCTTGGA
GGCTATAAAAGGGCATATTATTGCCCTTTTATAGATGGGAATTAATTTATTAGCAAACTCAGTTTCATGATTTTTTTTTTCTTTTGAGA
TGGAGTTTCGCTCTTGTTGCCCAAGCTGGAGTGCAATGGCACGATCTCGGCTCACTGCAACCTCTGCCTCCCGGGTTCAAGCGATTCTC
CTGCCTCAGCCACTGGAGTAGCTCGGATTACAGGCATGCCCCACCATATCTGGCTAATTTTATATGTTTACTAGAGATGGGGTTTCTCC
ATGTTGGTCAGGCTGGTCTCGAACTCCCAACCTCAGGTGATCCACCTGCCTTGGCCTCCCAAAGTGCTGGGGTTACAGGCGCGAGCCAT
TGTGCCTGGCCAGTATCATGGATATTTTTAAAGTGCTTGTTGTGTGTTGGGCACTACTATGTCCTGCATTTAGAAATAGAACTCATTCC
TATTCTCTGCTTTCCCATGGAGAGAGGCACAAACCTGCGGGTATAGGTGGGTATAGTTTCTTAATGTGCACACCATCGTAAAGTGGAGA
CCCTGTGCGTGAGATGTGTGCTGTGTACAGGGTGGGCAGGAGAGAGACTGGTGAGGGGATGATGCCCTTATGCTGGAAGGGGCAAGATG
AAGCCTCTCTGGGTTTGGTGGGACCTTGGAGACATCTGCCTAGTGTGTGGTCACCCAGAGGCCAAGGATGTGCTGTTTCTACTTGCTTG
CTGCTTTCTCTGGGAGTGGCCATTTCTTCCAAGGCAGTGTAAGAGCCTGGGCATAGGAGCCAGGCAGAACTGTGTGGGGATGTTGCCTC
CATAGCTTTCTAGATGAGCAACCTTAGGAAAGTTTGAACAGTTTCTCTACATCCTGGGTTCTTCATCCATGAAATGGGAATTATTGTTT
GAGCCTCTAAAGTGAGACAGTCAAATGAGATGGGCATTTGAAGCCTTCAGCAGGGTTCCAGCCTCACAGCTAACTCTCAACTAGCTGTG
ATGGTTACAGAGCTGTAGGTTGCTCATAAGTCAACATTGTAGTTGCTGGAGCCATCAACTCAGCTATAGGATACAACACTGAGCCCTTT
TCACTATCCTTTGATGGGTCTCCTTTTCCCTAGTATGGGCCATCGGCTAAGGTGATTGAAAAAGGGCCTTTCATGTTTTCAAAGGGGCA
AAGAAACTTCAGTAGTTGGTTGGGCATTTTATTCAGTAGAGGTGACTCCATGCAGTTCTTTTTGGGGGAAGGATGCTGCACATTCCAGG
GTTTAACAAAAATTTATTGAATGCTTACTTTGCCCCAGATACCTGTCTTGGTAAAGGTGATAAATAAGACAGCCTCATCCTCATCTTCA
TCAAGCCACCAGTCTGGTGGTGGTGACTGGGGTGGGGACTGCTGAGGCATTGGGCCCCTTGAAGCCTTCCCTCTGGGTGTCTTCCTCCC
CAGGATGGTCTGGGCTCTCTCGGGATGGGAGGAAGTTGGTCTGAAAGTGCCTCAGTGATCGCTGAAATAGGTTTGCCAAAGATAGAGGG
GAAAGAGATGGCCAGCCTTTGTTTAGGGACTCCCAGAATGGTCTTAGGTCAGGAAAGAGCGAATGGGTACATTTCTCCCAATTTCTGGG
CTGGAGATTCCAAACTGACTCCTTCATGGCAGGCTTCCTTATCTCTGCAACATTTGTTTACATGTTCCCTTCATAGATTTCTAACAGGA
TGAAGACTTTGTTCCTTGGGGTAAGGGTCTACCTCCAGGTGTGTGGGGAAAAGTTGAGAAGCTGCTAAATTCTTGGCTTTGACTTATCA
ATGGTTTTTCGGAGGGCCTGGAGCTGGGCTGGGACTGATCAAATTTGGTAATTGCTTAGGCAGCCTCCACTTTTGTTGCTCCTGGGGCC
AGATGGCGTTGGGCTAGAAACAATATCGGGGAGTTCTGTTTGGTTTTAACTGCTGTACCTGGGTGCTCTGTTATCCAACCAGGCCCTCT
CCTAAGTTCTCTTCTTTTTTTTTGAGACAGAGTTTCACTCTTGTTGCCCAGGCTGGAGTGTAATGGCGCTATCTCACAGCAACCTCTGC
CTCCCAGGTTCAAGCCATTCTCCTGCCTCAGCCGCCGTAGTAGCTGGGATTACAGGCATGCACCACCACACCCAGCTAATTTTGTATTT
TTAGTAGAGACGGGGTTTCTCCATGTTCGTCAGGCTGGTCTCGAGCTCCGGACCTCAGGTGATCTGCCCGCCTCGGCCTCCCAAAGTGC
TGGGATTACAGGCGTGAGCCCTGTAATCCCGGTGCCCAGCCAGTTCTCTTCTTTTTTTAAGCCTCTTCCTTTAGAAGGAAGAAAACATA
ATCTATATGCTGGCTGGCTCTACACAGCAGCAGGCATCACAAGGGGTTTTTGTCTGCTTTTGCTTCTGAAGGTGAACCCACTATCTATT
TAGAGAGGCCACGTCAGGGTGTGTGTGTGTGTATGTGTCTTTTTCCCTAGGGTGGGTATTTTCATTCANNNNNNNNNNNNNNNNNNNNT
GTTGTTATGATCCCCTGTTGTAGAAGAAGCAGTGAAGTTTTAGAGAGATGAAGTGCTTTGTCTCATGTCACACAACTGATAGATGACAG
AATCACATATGTATTCAGGGGTCTGGTCCAAACCCTAGAAGAAGGTGAGCCATGCATGGCCCGATGGCACAGAGCATGAGCTCTGGTTG
CACAATCGTGGGAGGTTACTTAATCTTGAGAAACCTCAGTTTTCTTATCTGTAAAATGGGACAGTATGTATCCCAAAGGGCCATTTTGA
CCATTTATCAAAGTCAGTTTCTCCTCCGTATGGAGCGGAGAACAGTGTCCAGCATGCAGTACGTGCTCTGATGATCTTGGCTGTAATGA
CCATCCTGGTTAGCGTGCATTTGGCCAAAAGCTGGAGTAACTGGTAGCTTCCTGATCTGAGAGGTGTCAGTGGGTGGTGTGGCCCAAAG
ATTGAGAATCTTGTCAGGGGTGGCCACGTGAGGGTGGAAGTGGTTTGAGTGAGGAATGGTCTTGAGGTAAGAGTTGGACAAACAGTGTC
TTCTCTCTCTCTCTCTTTTTTTTTTTTTTTTTTTTTTTTACTTTTTTTTCAGATGGAGTTTTGCTCTTTTTGCCCAGGCCAGAGTGCAA
TGGCCCGATCTTGGCTCACCACAACCTCCGCGTCCCAGGTTCAACTGATTCTCTTGCCTCAGCCTCCCGAGTAGCTCCGATTACAGACA
TGCACCACCCACGCCCGGCTAATTTTGTATTTTTAGGAGAGATGGGGTTTCTCCATGTTGGTCGGGCTGCTCTCCAACTCCCGACCTCA
GGTGTTCTGCCTGCCTTGGCCTCCCAAAGTCCTGGGATTATAGCCGTGAGCCACCCCACCTGCTCGACAGTGTCTTCTCTAAGCAGAAC
TGTACCCCTGAAGCGCCCAGGCTAGAACTCTCTATTGAATGAGGATCACTCCATTGTGCCAGCTGCTCTCATATTGCTTTGATGGCCTT
TCTCAGAAAGGAGAAAGGTGGTGCTGGGTGTGGCTTTGGCTTCCTATGGCCGCCCCAATGCTGGTCTGACACTTTCTGAATCTGGTTTG
TGGTTGGATCTGTGCATCCTACCGCTCCACTCCATGTCCTGGCCTTGGAGAAATCATTTCCCTCCCGCCGTGATTGTTCACCTTCAAAG
GTCAACAAAGGCAGTAGGCACTGCTGAGCTCTTGCAGCCTTCAGGGCTCAGCTGCTTCCTCTCCTGCTTCCAGTGAGGGGTTGCTTGAG
GGTGCCTTTAGGAACGAACCTCACCCCTAATGTAACTTGAGCAGGTCTTTGCTCTAAACTCTTCGCTGGGAACATACCCTCCCGTGGGA
CTTGGCATGGCACCCACCTCTGCAGAGCCACCTGTGCCACAGAATAACCTCTTCTCCCTAGACACACTGTGTGTCCTTGGTTGGATTCT
CTAGATGGGAGGGACCCCCTGAAGCATAACTGTGTATGTTCACATATGTGCCTTGGAAGAGTTTTATGCAGGAAAGGATTTGTAAATCA
GAAAAATGAATGCTGGTATCTAATTTCCTGTCCCCGTTTTAAGTCCAGACCAAGTCACCTAACCCCTCGGGCTTGCAGCCCGCATCATC
CACTGGGTGATTACCTTGAGTTATCTGGTTCAGTCCCTTTGCTGTGGGCACCCTCTTTACCTCTACAAGTAAACTGCAATTAATCTGAC
AGCAGGGCCAAGGCAACTGACGAGTCTTGCGTGCAAATCTTGAGATCCTTTGGCACATTATCTAATTCATTGTTTTTCTTTTCTTTTTT
CTTTTCTTTTCTTTTTTTTTTAGACAGGCTGGAGTGCAGTCGCACGATCTTGGCTCACTGCAACCTCTGCCTCCTGGGTTCAAGTGATT
TTTGTGCCTCTACCCTCCTGAGTAGCTGGGATTCCAGGTGTGTGCCATCACCCCCAGCTAATTTTTGTATTTTTAGTAGAGACAGGGTT
TTGCCATGTAGGTCAGGCTAGTCTACAAGTCTGGCCTTAAGTGATCCACCCTCCTTGGGCAGGGTGCCACCTGCCTGAAAGAAAATGCC
CACCCTAGGGAAAAAGACACACACACACACACACACACACACACACACAGAGAGAGAGAGAGAGAGAGAGAGACAGAGACCAACGTGGC
CTCTCTAAATAGGTAGTGGGTTAACCTTCAGAAGCAAAGGCAGACAAAAACCACTTGTGATACCTGCTGCTGCGTAGAGCCAGCCAGCA
TATAGATTATGCTTTCTTCCTTCTGAAGGAAGACTCTAGGATTACAGGAGTGAGCCTGGCCCCAATTCAATTCAATCTTTATGGGCATC
GAACAAGGCCATTATTTATTACCCATGCTTTACAGAAGTCAAACCCAAGTCTCAGAGAGGGAAGACATTTTTTAGTCTCACTGCGAAAG
TGACAGAGCTAGAATTTAGTCTCTGTCACGCTAAATCCTGCTACATGCTCCGTCTAGTTCAGAGCCTGTCTGCAGGTCGACACTGGAGT
CCCTCAGTTTTCCTCATCAGGCTCCTGGTCTGATTCTTCCAAGATCACTCTCTTTCCCTCAGCGGAAGCCAGCTGGGAATCACTGGCCT
TGTAACCTCTTCAGGCGACCTCCTTGGGATGTTCACAAGGAGCATAGACATCTGGAGAATGATACTGTTTTTTCCTAGGGCTGGGATGG
GGGTGGTATCCCTCACATCATTCCAGAAACAGCGCCAGCCCTGCTTTTATCAGCGGGGGCCACAAAGAGGCGACAAGCCCTGATCAAAT
GTGAGCGAGATTGACAATCTCAGCACTTTCCAGGGAAAGCATCCCGTCCCACATGGCTTCACGCCTCCAAGGAAGCCCCATCCTGAGAC
AGAGAGACCCCAAATCACAGTGTGTTTCCTTTTGGCCAGAGATGAGGCTTACTCATCTCAGACAGCTGTGTCCACGGCATGTTCCTCAA
GGACAGCTGCTCCAGATGAAAAGAAGAGATGCTCCCAGAGCCTTGAGGGGCTGCTGATAGGAACCCAAGGCCTCACCCTGGGGCTGTAT
CCTGTGGCTTCAAAAATATATGTATTTGAAAGCTTCTAGCAAAACAGATTACACAAGAACGCAAATGTAAGATCCCTTTGGGATTCTAA
CTTTTTTTCTTAAGGTGTTTAAAGAAAAATGACACCCTTAAAAAGAAATGCCATTTGTTTTCAAAGCTGCAGCACCAGAGGACATGGAA
GAGAAAGGCTGAAGTGTCCCTGAAAATGAGTGAAGATTCCAGTCTGTTTTTACTAATGTCCAAAATCTTAGGTTTATTGCCTTTGCGTT
CCTCTGCCCCAGGGTCTCTGTGTGGCAGGCGACTGGCTCTCCTTCCATGGTGCAGTCATCGTCTGGCGGAATGCATGCTCACACACGGA
AGTTGGTTGAGCCTCTCCTTTGAGCAGTCCTTCCTCTGGTGACGTGCACAGGCGCCCATCATGCCCATTTCATAGATGAGGAAACTGAG
GCTGAGAGGGATGCTGCAATTTAGGACTTCTGGGTGATATCACAGAAGCCTCATTCTCTTTTGTGGCATGTACCTTCTCTATTTGGGAA
GTTAAATTGGACTATGGGGATTGGACCAAGGGGTCTTCTTGCTTCCGCTTTCTCTAGGCCAAGGCTTTGCAGTTATGGAGGAAGAAAGA
GCTGGGGTCTGTCTTGGCAACCCTGTGACTCTTCTCCCCTATCCTCACCTGCCTTCTCACTTGACCAACATATTTTAAGGGCTCCATGT
ACAAAGGTAGGGCTGTGCCCCTTTCCCAACCAGCACCTTGCCCCTGTACCCTGATATGGGATGGTTAAGGCACACAGGCTTCATGGATG
GGCCCCTACGACCCCACATGGAGCAGACCTGGACAGGGTATGGTCCCCCAGGTGGAGGTGCTGGAGAAAAGCCGCGTTAGGCAGCACGG
GAGCCAGGTGCGGGGTCTCTCAAAGCCTCGAGTCCCTCCCAGTGCTGTGCTCTGTGAGGACTGCTTTTGACTGCAGGGCACTGACACTC
ATACCTTTTGCTTCTGCAGCTCACCATACTCCCTCGAGCCTTCTCCCGACCTGCGCGGGTCACCTTCGCACATACAGCCATCATGATGG
TACTTTAAGTGGAGGCTGAATCATCTCCCCTTTGAGCTGCTTGGCACGTGGCTCCCTTGGTGTTCCCCTTTTACTGCCAGGACACTGAG
ATTTCGAGACGTAAGTGGCTTACCTGAGGCCATGTCCTAACAGAGAAGATGAAGAGATGATTGAAACAGGCCTAAGACCAGACCTAAGG
GTCTGTACATTTTCCACATACTTTCCATATCTTTAGAGGCCTGACCAAAGCAGATCTTTTCCTTTCTTCTAGGTAAGTCCAAAGGCACC
TGCCTGCTGGGCCCACTGTTTTCTAACTTTCCTAACTTTCTGATCCCTTGGAGGTGATAATCAAATATTCTAGTCTGAGGCATTGGGAT
ACATCGTGCTAGGTTCTGACACTCTGCGTCAGGCCTCAACCCTGCATTTTGTGGAGGTGGGTGGGAGAATGTTCCCCTGGGGAACATGC
CTAGACACGGGGGACAACAGTTGCCCTCATGCGGAGGTACCTGTTTACTCGCTCTTATGCGACCGCTTTCACAAAACCACTGCAGGTGA
GTGAGTTCCTGCTGAATATCAGCCCTGGTGTCTCTAGACTCATTATTTCCCCCACCCAACCCCTATGTTAGTTCATCTCCAGCCACATT
TTTATTGCCATAATCCAGGCCTGGACAGCCCAACATCTTTTAACAATTTTAATTACTGAAAATAATAACTGCATTTTTTTTAAAGCCCA
ACTTTTGGTAGAGTCAGCCCAAAATACAGTCTTTGTGTTCCCATCTGGGAACTGGATTTGGAATTGTTCTTCCATGAGACTGCAGAGCA
GAACCGCAGGGCCAGAGGTCCCACGAGCTGGTCACACCCGGTTCTGCTCCTTGCTGGCTGAGTGACCTTGGGCATTGTGATTCATATTC
TCATCTCCCTTCAAGTGTGGTTACGAATGCTTTTTATCCTGCTTATCAATTTCTATGATTGCCATAAAATATTAACACAAATTTAGCAT
TTATATAACATAATATATTAATGCATAGTTTTTTTGTGGGGGGGGGCGGTGGAGGGGCAGTGTTCAGACAGAGTCTTCCTCTGTCACCC
AGGCTGGAGTCCATGCTGTGATCCTGGCCCACTGAACCTTGACCTCCAGGGCTCAAGGGGTCCTCCCACCTCAGCCTCCAGAGTAGCTG
GGACTTCAGGCATGAGCTATCACACCTGGCTAATTTTTTGTATTTTTTGTACAGACGAGGTCTCACTATCTTGCCCAGGCAGGCCTGGA
ACTCCTCGACTCAAGCGATCCTCCCACCTCGGCCTTCTAAAGTGCTGTGATTACAGGTCTGAGCCACTATGTCCGCCTCACGTCCAGTT
ATCTAGGGCACAGGTCAGACATGGATCTCTCCAGGCTAACATCAAGGAACCTGTAGGCTGCCTTCCATTCTGGAGACTGCAGCGGAGAG
TCTGTTCCTTGCTCATTCTGGATGTTGGCAGAATTTGTTTCCTTGAGGTTCTAGGGCTGAGATCCCGTTTTCTTGCTGGCTGTCCAGAT
CCATTGAGCTTCTGGAAGCCTCCTCGCTACGCTTGTGGCCTCCTCTCTCCATTTTCAAGTCCAGCAATGGTGGGTCCAGTTCCTTTTAG
GATTTAAATCTTTTCTCCCTCTTTCTTTTCACCTCTGTGATCTCAGCTAGAAAAGATTATCTAATTTTAAGGATTCATGTGATTGGACT
GGGGCCACCCTGATAATCCAGAATAATCCCTACTTCAAGGTCCTTAGGTTTAATCACATCTAGGAAGTTCTATTGCCATGGGAGGCAAC
ATATTCACAGGTTCCAGGAAATTCCGACATGGGCATCTCTTTGATTGGAATGAGGGGTGGCAGGTATTCTGACACAGTACCTCACAAGG
GTTGCTGTGATGATGAAGTGAATGGAGACACGTAAAGCGCTTAAAATATTATGTAGAGCCATGTGAATTTGGCAGCATTGAATTGTTGA
CCTATAAAAGGTCAACTTAATAGTATCTGGTCTATAGTAAATCTTCAGTAAATGTTTCCTCTCATTACTCTAGTGGTAGAAATGTCATG
CTTTATTTAGCCAGTTTGCTATTGTTGGGCATTACTTTTTAAAGAGTTATTATTTTGTTATTATAGTATATATGTGAACACCGTGATCA
TCATGCCTTTGCAAATTTTATTCTGATTTAGGGCAATGCATGTCTTTCCACGCCACACTCAGTCTCCTGCTCTGGTGCCAGTGTCTCTG
TCGCCTGAGGGGCCCCTCTTTCTTGCAGAGCCCCTCATCCAGCACCAGCTCAGCTCTGCGCCATGTGTGTGGGGTGGGACTTTTCTCTT
TTGGGAAGTGGGCCATGCCTCTCCTTCCTGAGACCATCCCCTTTGGCTTCCTCGGCGCCCTTGGCTCTTTGAACCAGCCTGCAGGGCAC
CACACCTGTGGGCCCATTTTTGTCAGGAAACGACTCCCTTCCCAAGGGCACCCTGAGTCCCACGTCCCCAGGCCCACAGATGTTGGGCC
CTCAGAGTGTGATCCCATGTCCCCAGCTCTGTAACTCCCTGCATGTGGCTCTTAAGACTCACAAGCCCATGTTTGCCTTGGCCAGACAC
TTTCGCCTGTCAGTTCTGCCAGTTGGCAAGTGTCATCCCCACTGTGTGAATGTGGAAGGGATAATGAAGAATTTCCACGGCTTGCCTAG
GGTCGTTGAGCAATGTGGAAGGGATCATGGAGAATTTCCATGGCTTGCCTAAGGTCATCAAGCTGGCATCTCAACCCAGGTTCATCTGC
CTGTCATGACAGGAAAGAAATCAGGTGTGATTCTGTTCTTGTTGACCCATAGCCACCTCTCTCATTAAGGCTTCTCCTGCAGGCAACTA
CACAGCCAGTGGTGGGAAAGCACCTAATTTACATCTTCATGCATTGAAACTGATGTCACTCACCTCTCCCCCTTGACCTGGAAGAGCCC
CTGAAGGCTGCCATAGGGCAAATGATAACCCATTCTACAGCTGCTGAAACTGAGGCCCAGGGAGTGTCCAGACCTTTCTGAGGTCCACC
ATGTACTCTTGACCAGGATGAGTGATAGCCACCTGGTGACAATGTTGAAGGTTCTCATTCAGTAGAGCTGGGTTCAATTCTCACTTGCC
TGACCAGCTGGTGCCTCATTTTGCTCTAAGTATTGGAGCCTCCAGCTTTATTTTCTTTCAGTGGGAAAGACTGTGAGATTCAGTCCCTC
CTTGAAGCTGCGCATCGACTCTCCTCTCCTGGGTACTCTTTAGGTAGTGGCTGTTCTGAAGTGGCTTTGGAAACCCTGGAATGAAACTG
TCATCTCCAATGCTGAGATTTGTATAAACTGTCATTTTAACTGTGATTATTATTCCTTAGGATTTGAGAAATAAAATCTGAGGAGGGAG
CAGGACCTGCCTCAGGGCCTCCTCTCCAACTTGAGTGACCCATCTTCCCAGGTCTCCTTTTGTTCTGTCTTGAGCCTCTCTCCTGAGGT
CCTGAAACAAGAGAATGTGCTGTTGTAGGGGGCCTGGAAATGTCCCATGGACCATCTCGTGACTCAGTAAGATGATGTGTGTCCTCTGG
GGACAGAAGGAGGTCCTGCACAGAGTGTTCCTGCCCGTTGACGCCCTCCCCAGATCCTAACGCGAAGTCTGACACATAGTAGCCTCCCG
GTAATCTTAATAAGCAAAAGGACACATTTCTCAAGTGATCTTAGCACCATTTCATGCAATCAATTCCAGCCCAGCCCTTTATTCTGGCC
AGCCCTGGTGGGTCCATTAGCTGCAAAAATTAGTCGTGCTCAGTTTTAATGCAAATGACTAAGTATTACTTTATTGAATTGATTAATAA
AGAAGCTCTCTTTGTATGATATCAAGACCATCAAGAAATTGCAAAGACATAGGTAGGGTCTACAGCAGAGTTTCTCAGCCTTGGCGCTG
TTCATATTTTGGGTCAGATGATTTGTTGTGGGGGCTGTCCTATGTATTGTAGGATGTTGCTCGGCATCTCTGGCCTCTACCCACTGGAT
GCCAGTAGCTCTGTCAGTCGCAACCCCAGCTTCCAGTTGGGACAACCTAAAGTGTTTCCAGACATTGCCACATGTTCTTTTGTGGTGGT
GTTGGTGCTGGTCCGTACATATGTGTGTGTAAAAATCTCCCTTTGTTGAGAACCACTAATCTAGAGACCACCTAATTAGGGCTGACGTG
GGCTCTTCCACAGAGCCTTCTCCTTAAAGACAGATAGGTTGATGGACACTTGGATTTAGGGAAAAGAAAACTGAGTTGAGGGTTTAAAG
TGAATTTCATCCCCAATGTGGAAGATGAGCGTTCCGTTCCTGGCTGTAGGATGTGGAGCATGTTACTCCACATGTGGGAGAAAAAAATC
ATCCCATCTACTTCAGGACTTTTGTGTGATTAAACAGGCAACATAGTCTAGTGCTTTTCATTCCATGCGCACTCAATAAAAAATAGGCG
TTATATTTATGAATAGTAACAAAAAGCCTCTTAAGGAAAATTATTTTGGGCCCTTGTTCAAGGTTCAGTTGACCATTTATTTTTAAAGA
GATGGGGGTGGCCCAGGCTGGAGTAGAGTGGTGCCATCCTAGTTCACTGCATCCTCAAACTCCTGGGCTCAATCGATCCTCCTGCTTCA
ACATCCCAAAGTGCTAGTATTATAGGCATCAGCCATTACACCTGGCCCATTTGACTATTTTTTCTGCTTGATTGTAGAGAAGGGAATCT
AAGTCATGAAGATCTAAACTATAAAACCTGCGTGGCTTAGAGGCTGAGCATGGATTGTGATTTAAGGAAGCCCCAGGTTCAATTTCTGG
TTCTGCCACTTGCTAGCCATGGTGAGACCTTGCCCAGCTTTCATACTTTCAGGTTCTTTGTCTGTAAAATGGGAACAATAAGAGTACTG
ACCTCACCCGGCTCTTGTGCTACTGGCTGTGCTGCCTGAATGGGCCCTCCTTTATTTGAATCCCTCTCCCACAGTTGTCCCATTTTTTT
TTTTTTTTAAATGGTCACCCAAGTTCCATTATGGACGATCAGGCATTCAGGGGAAGGAGGGATCCAAAGCAGAGACAAAACCAGCCAGC
CAGCTCATCCCCTCTCCAGCTGATACCGAGGAGCAGGATGCCAGTGGGAAGAAGCGTGGCCATATGGCTGGCAGGATGCCTCACTGGTA
ACAGTCAGCCCGGCCCCTTTGAACTGGTTTTAAGACCAATTTGGTAACTTGATTTGGAAAGTGGTATCGATGACTCCGAGGGCGCAGGC
TGCTCATTAGCTGCTGTCTGGGCCCGGCAAATTGGTTTAAGTGGAATGAAATTGGTTCCTTCCCTCCCCGTGCTCATGGCTGAGGTCCA
GGCCCGATTGTAATGGGAACCTCTCCTGGGCAAAGGACATAGAAAAGCTTTCCGGGGAGTCTGACCCTCTTTTTTCATATCTGAATTCT
ACTTTCCCCTGCGGCTCAACGCAATAGCCTCATAAAACATAAAAATCAACCTGAAGTGGGTGGGGTGAGTGTGTGTGTGGGAACTTGGA
TCAGAGCACAATCAACAATGAACAGTGGCCGAGTGTTTTTCAAGGGCCAGTTAACCATTCCCCTGTTGAGACACTGTGCTTGATGCATT
TTTTTTCTTTTCTTTCTTTCTTTTTTATTCTCCCATATTACAAACACTGCTGGGAGGAACATCCGCACATGCTTTCTTGGTCACTTATG
TGAGAGCTCATCCACCATTTATACCCTGACGCGACTTCACTGCCGTCGAGGCTAAGGAACATTTTCTGTTTCATAAGACGCGATCACAT
TGCCCTCCAGAACAATCATGCCAGTTTACACTCCCACTCACTGTACACAAGAGCCCTGTTTTCCCCCATCTGGTCACCCACATTTGATA
GGTCAGAATTTTGCTTTTTCCCAGTCTCATCCCGGTCATCCCATTTCTCGTAGTTCCCCTGATTCCCAGGGCGATCACCACCCTGCCCT
CAACTGCCCCATGCTGAACGCCCTCCAAACGCAGCATGAATCCTCACAGTTCCCAGCCTCTGCCCTGACAACCTAGGAGGACACGCTGG
TCTGCTCCATTGCTTATCTGAAACGATTCCCTTTCCCTCTTCCTTTTGTTGCCTTGAGTGTCAGTTTTGGACCTCCCACTGAGCAGTTT
TTTCTCTCACGTAGCCATAGACAGGACTGAGTTCTTTGGAAAATAATCACATCCCACTGTTGTTCCCATTCGTTCATTTTTTCCTTCAA
CTAACCAACATTATTCAGCACTTTACTCACCACAGGATGTCGAACCGAAGGCCTGCCTTTTCCGACACTCACCGTCCCCAGTCAGCTCT
GTGTCCTAGGGAGAGACCCATGAGCTCTCTGGGCCTCCTTTTGTTTCTCTGTAATAAAGGACTAAAGCTACCAGCCTCGCTGCGGGTTG
GAAAAAAGAAAGAGTTAGTGTAACACATGTCAATGTGTACGAACGGGTTTAGAGACAGACAAAACATTGCACCAAGGCAGGGCTCGCTG
GAGTGCAGACCCAGGAAAAACACTGCAGGCCAAATTGCTTCTGGTCTGTCAGGGTACCAGCCACACATCCAACGCAGGGAACTTAGGGC
CATTGGCCTTTCTGCTCATGCTTACTCTCCCAAGCTTGTTGGGTTATTATCATGCACAAGCCATTCAATCCATTGTATTGGGTGCCTAA
TATATCAGACACTCTTAGGTCCTAGCGATGTAAGAGCAAGCAAAACAGACATGGTTCTGCCCTTGTGGGCTGTATAGTCTAGTGGAGGA
AGGGGGAAGAGGTTAAGTATTTGTAGGAAGGTGAACCCCATGTTAAGAAAGCATCTTCCATCTTTCAAATGTTTCCAAGTAGCTGGTTA
TTCATGATAGCGAGGAAATGGTGGTCATCTCCATTGCTCTGGTAACTAGTTTGTGTCTGAGAAAGTGCTGATTTCCAAGAAAAGGAAAG
AGACCACGGATGAATGATAGCAAGACTATTAAACAGGAATACAAAGAAGAGCTCACATTTATTGAGCACCTACTATATGTGCTGGACCC
TCTTGTAAGAGCTTCATACAAATTAGGCAGACAGGCAATATTATTATCCCCATTTGATCTACAAGGAAACTGAGTACAAAGTGATAGCT
TTTCTGCAGAGCTTGTGCACAGGGGCCAAAGTTTGGGAACAATGCCTTTCTTTAATTTGAAAGTATCTGTTGTTGGTGTCACTTGTTGG
AATTCCTAGAATCCCAGAAAAGTAAGTTTAAGTTTTCTTCTGCCAGCATCCGTCAGCCTACCTTTGACCCAAGGTCACGACTCCCTCCC
TCCTCCCTTCCCTTCCCTGCTCTGACATTCTTACCTTGTTTTTGACTTCCTTATTCCCTAGCTTCCTCCAAGCAGCAGGCTCTGGGAAC
AGTCCCGTTCATTTATATAACAAATATTTATTGAAATGCAGACTTGTTGTTTTCATAATACCTGTGTACAAGTTAAACCCGTGGTCACT
TGTTCATGTGCCTCTTGTTTATGACAAGAGGTGGTCCCTTGTTGAAAGCCTGTCACATGCCAGGTACTCAGTCTTTTCTTTCCTTCTAA
TAAGTAGGTGAGTATTTTCCTTCCTTTGCACACGAGGAAATGGAGGCTCAGAGAGCTTAGGTAGCTTACTCAAAGCCACACAGCTGGGT
TAAATGGGTTCAGCACCATGAAAATCTGACCTTTAATGTCTGCACTCTTATTCTCTGGCTTATTGGGAAAGAATTGGACAGGGATGGTA
AATGGAGTTGATGGAGGCAAAGAGATAATTACTGTTGCTCAAAGGTGGATCGAGAGAGTTTGGGCAAAAACGGAACCACGAACAAGCCG
TTGAAAGTCCCCTCATCCTGGACCTGTGGTCTGTCCTTTGTAACTGCTCTTTTTTTGGCATTCCCTCTGTCTCCTCTGCCCATCCCAAT
GCCAGGCACACTCAGCCTGTAGGGATTTACAAGGTGGGCCTCAGGTTTCTCCCCTCCCAGGTGGGCATAGGAATCACTGCTTAGCTTAG
ATTTGGCAGAATGAAAGAGTCAGGCAGAGTCTCACCCGGCCCTGTTTCTTAGCTGCTTTGACACCTGGGGCAGGTTTTTGGACCTCTCT
GGACATGGCCACCTGTGTTGTCACTGCCCACCACAGGCTGGGTCTGTGAATGGAAGACTGAAGAAAAGGCGATGATCACAGTCCAGGGA
CAAGGTTTCTGAATCTTGTGAAATAGTTACAAATACTATGAAACAGGAATACAAAGAAAAGCTCACATTTATTAAGCACTTACTTTGTG
CTATACCCTCTTGTAAGAGCCTTGTACAAATTAGGCAGACAGACAATATTATTATCCCCATTTTACCTATAAGGAAACTGAGTGCAAAG
TGACAGCTTTTCTGCAGAGCCTGTGCACAGGGGTCAAAGTTTGGGAACACATTGCCTTTCTTTAATCTGAAACTATCTGCTGTTGGTCT
CATTTGTTGAAATTCCTAGAATCCCAGAGAAGTAAGTTTTAGCTTTCTTCTGCCAGCCCCGGTCAGCCTGCCTTTGACCCAAGGCCATA
GTAAGTTCCAGAGTGACTTCCCGTCTGTTTTATTATCCCTGACAAAGGGATACAGTATTGTGCGGTGGCTGTGCATGGCGTGTAGTAGT
GTTTTCCTTCTCTTCAAAAGCAGCTGCTGAGTGGTGGAGCACAGAGGTATTCCCTGAGCCCCACCTTGTGCCCATGGTAGGTCCAGGGC
GTTTGGGGGAAGGAGACCGACTCTAGGTGGTCCCGTCTGGTTTCTCAGGGGAGAAAATTCATAGATCCATCTCCTAGCTGTCATCCAAT
TCTGTGTAGAAGGCGAAAGCCAAGGTTGCCCTCAGATTTTGATCTTGGTTCACTCTGATTTCTCTATGCCACACTAGTAGAGCTTCAGG
GAAGCTTGGTGACTATGACTGGATGAATTCTGTCCCCCTTAAAACCATAGGTTGAAGTCCTACTCACCCATACCTCAGAACGTCACTGT
GTTCGGAAATAGGGTCTCTAGAGAGGTACAGTTAACTGAGCTCACGTCGCGGGCCTCCAATATGGTCAGACTGGTGCCTGTCCTTATCA
GAAGAAGAGATTAAGATGACAAAGGAGCAGTCAAGACCACGTGTACACACAGGGAGGAGGTGCCCATCTACAAGCCCAGGACAGAGGCC
TCAGAAGAAACAACTCTGACCACACCTTGATCCTGGACTTCTGGCTTTCAGAATTGTGAGGAAATAAACCTCTGTTGTCTGAGCAACCC
AGCCTGTGAAAATTTGTTGTAGCAGCCCTGGCCGATCAATACAGTGACCATGAGCAAGTCACTCACCCTCTCTGAACCCCCTTGGTCTC
AGCTCTAAAAAGAAGCCTGAGAATTTCTGCCTCACAGGCTGTGGTAAGAGGCATCAGAAATAGCCGACTTTCATCGGAGGCTCACTGCG
AGCCAGGGCCTGTTTAAGCCTTTTCCATGTCTGAACACACTTCATCTTCATGTGTAGCCTTTGGCTGGGCAGGGCAGATACTATTGTTC
CCATTTTACACGTGACCAGAGTGGAGCACAGAGCTGTTAATTAACCTGTCCCATGTTAGTCAGCAAGGAAGGGGCTGGTCCATATGTGG
AACTTCGTAGTTGTACTCTGGTGCCCACACACCAGATCACTATGCCGTCCCAGCTGTTGTTACAATAAGACTATGTAGGTGAAGGGCCC
ACCCAGTAGTGCCTGGCCTATGGGACACATTGAAGACCAGCCCCCTGTGACCAGAACCTCCCATTTGCTGCATTGAGTTTGGAATGTTT
GAACTCCAGGAGCTGTGTGTGTGTGTGTGTGTGTGTGTGTGTGTGTGTGTGTGTGTGTGTATCTGTCTGTCTCCCTCTCTCTCCTCTCT
CCCCTACCTTCCTTTTCTTCCCTCCTGTTTTTGGGAGGGAGCTCAGCTTCCCTTCACTGCCACTGCCCATCAGCATCCTCTCCCTATTC
AGCCACCCCCACCCCCCATCCCTGGTATTAATTGACTTTGGCTCCTGTCCTGGAGCTCAGCCTGGCACCCAGACCTGCCGTGCTATACT
AACACCTCTCACATGGTTAAACCAATGGGGGTGGCATTTAGCAGATGACGCTGAAACCCGTGAATCTGCCCTTTCTCACTGGGCCCTCA
CTGCTCTAGAGTCCCCGCCTCTTTTACGAAGCGGCTGTCATGGTTGATGGGTGAGTGTCAGGCCCCACCCAGGGACAGGGAGGCCCAGT
GCCGGCTGTGGAGTTACCCAGACCTTACGGTAAAAACGCATCACCCTGAATGGGGGCCAGGCCAGGCATCGGCCCAAGCCGGCCCTGCC
CAGCGGCTGGATTCTGACTCGCTCTGCCTTAGTCACAGCCCAGAGACTCTTCTGCTTCCAGCTGAGGCTGTCCCCTGCCTTCCCTGGCG
GCCCGGGTTCCTGGAAAAGCTGTGTGCCAGAGGACGGGAGTTTCCCTCCAACTTAAGCCATTAGAAAGCTCATTTATTGAGCACTTACT
AAGTGTCAGAGACTGTGCTAGGCACTTTACATGAATCTTCTCATTTAATCCTTTTAATAAACTTCCGGGGCACATGCTCTTCTAATCTC
CCTTTTACTGATGATGCAGGCTTCACAGTGGGAATGCTAACCGCAGAAAGCAGCTCACCTCACTTGGGTGCTACTGTGTGCCAAGTCCT
GGGGTTAGCACCCAATAGCCCCGTGGCGGCATGCTGTTATTACTCTCCCTGCATAGGAGTGGGAGGCTGAGACCTGGGTGTTTCTGGGT
GTCCCAGCTCCTGTATGCCATCTGCCTGTCCAGCCCTTAGCCACAAAGCTGTTGTGCCATGTGGGGAGATGCGGCCAGTTTCCCAGGTG
AGGCTGGTCCTGCTTCAAGCG

| | |
|---|---|
| Human PVT1 Transcript | (SEQ ID NO: 31) |

AGCACATGGCCCCGCSGGCCGGCCGGGCTCGGGGCGGCCGGGACGACGAGGGGCGACCACGAGCTGCGAGCAAAGATGTGCCCCGGGAC
CCCCGGCACCTTCCACTGGATTTCCTTGCGGAAAGGATGTTGGCGGTCCCTGTGACCTGTGGAGACACCGCCAGATCTGCCCTCCACAG
GCCTGATCTTTTCGCCAGAAGGAGATTAAAAACATGCCCCTCAACATGCCTGTGCCTGTCAGCTGCATGGAGCTTCGTTCAAGTATTTT
CTGAGCCTGATGGATTTACACTGATCTTCAGTGGTCTGGGGAATAACGCTGGTGGAACCATGCACTGGAATGACACACGCCCGGCACAT
TTCAGGATACTAAAAGTGGTTTTAAGGGAGGCTGTGGCTGAATGCCTCATGGATTCTTACAGCTTGGATGTCCATGGGGGACGAAGGAC
TCCAGCTCCCTGACACCGTTGAGATCTCTGTTTACTTAGATCTCTCCCAACTTCCTTTCGCTCTCCCTATCGAATGTAAGACCCCGACT
CTTCCTGGTGAAGCATCTGATGCACGTTCCATCCGGCGCTCAGCTGGGCTTGAGCTGACCATACTCCCTGGAGCCTTCTCCCGAGGTGC
GCGGGTGACCTTGGCACATACAGCCATCATGATGGTACTTTAAGTGGAGGCTGAATCATCTCCCCTTTGAGCTGCTTGGCACGTGGCTC
CCTTGGTGTTCCCCTTTTACTGCCAGGACACTGAGATTTGGAGAGGTAAGTGGCTTACCTGAGGCCATGTGCTAACAGAGAAGATGAAG
AGATGATTGAAACAGGCCTAAGACCAGACCTAAGGGTCTGTACATTTTCCACATACTTTCCATATCTTTAGAGGCCTGACCAAAGCAGA
TCTTTTCCTTTCTTCTAGGTAAGTCCAAAGGCACCTGCCTGCTGGGCCCACTGTTTTCTAACTTTCCTAACTTTCTGATCCCTTGGAGG
TGATAATCAAATATTCTAGTCTGAGGCATTGGGATACATGGTGCTAGGTTCTGAGACTCTGCGTCAGGCCTGAACCCTGCATTTTGTGG
AGGTGGGTGGGAGAATGTTCCCCTGGGGAACATGCCTAGACACGGGGGACAACAGTTGCCCTCATGGGGAGGTACCTGTTTACTCGCTG
TTATGGGACCGCTTTCACAAAACCACTGCAGGTGAGTGAGTTCCTGCTGAATATCAGGCCTGGTGTCTCTAGACTCATTATTTCCCCCA
CCCAACCCCTATGTTAGTTCATCTCGAGCCACATTTTTATTGCCATAATCCAGGCCTGGACAGGCCAAGATCTTTTAACAATTTTAATT
ACTGAAAATAATAACTGCATTTTTTTTAAAGCCCAACTTTTGGTAGAGTCAGCCCAAAATACAGTCTTTGTGTTGCCATCTGGGAACTG
GATTTGGAATTGTTCTTCCATGAGACTGCAGAGCAGAACGGCAGGGCCAGAGGTCCCACGAGCTGGTCAGACCCGGTTCTGCTCCTTGC
TGGCTGAGTGACCTTGGGCATTGT

| | |
|---|---|
| Human PVT1 Protein | (SEQ ID NO: 33) |

MGPRAGRARGGRDEEGRRRAASKDVPRDPRHLPVDFLAERMLAVPVTCGDTARSALHRPDLLARRRLKRCPSRWLCLSAAWSFVQVFSE
PDGFTVIFSGLGNNAGGTMHWNDTRPAHFRILKVVLREAVAECLMDSYSLDVHGGRRTAAG

Certain aspects of the present invention are described in greater detail in the non-limiting examples that follow.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all and only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

### Insertion Site Analysis Following Tumor Induction in Mice

Tumors are induced in mice using either mouse mammary tumor virus (MMTV) or munine leukemia virus (MLV). MMTV causes mammary adenocarcinomas and MLV causes a variety of different hematopoetic malignancies (primarily T- or B-cell lymphomas). Three routes of infection are used: (1) injection of neonates with purified virus preparations, (2) infection by milk-borne virus during nursing, and (3) genetic transmission of pathogenic proviruses via the germ-line (Akvr1 and/or Mtv2). The type of malignancy present in each affected mouse is determined by histological analysis of H&E-stained thin sections of formalin-fixed, paraffin-embedded biopsy samples. Host DNA sequences flanking all clonally-integrated proviruses in each tumor are recovered by nested anchored-PCR using two virus-specific primers and two primers specific for a 40 bp double stranded DNA anchor ligated to restriction enzyme digested tumor DNA. Amplified bands representing host/virus junction fragments are cloned and sequenced. Then the host sequences (called “tags”) are used to BLAST analyze the Celera mouse genomic sequence. For each individual tag, three parameters are recorded: (1) the mouse chromosome assignment, (2) base pair coordinates at which the integration occurred, and (3) provirus orientation. Using this information, all available tags from all analyzed tumors are mapped to the mouse genome. To identify the protooncogene targets of provirus insertion mutation, the provirus integration pattern at each cluster of integrants is analyzed relative to the locations of all known genes in the transcriptome. The presence of provirus at the same locus in two or more independent tumors is prima facie evidence that a protooncogene is present at or very near the proviral integration sites. This is because the genome is too large for random integrations to result in observable clustering. Any clustering that is detected is unequivocal evidence for biological selection during tumorigenesis. In order to identify the human orthologs of the protooncogene targets of provirus insertion mutation, a comparative analysis of syntenic regions of the mouse and human genomes is performed.

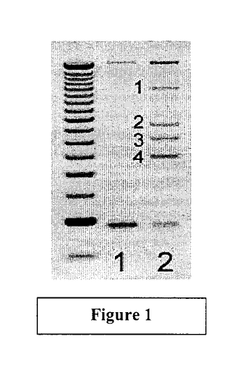

An example of PCR amplification of host/virus junction fragments is presented in FIG. 1. Lane 1 contains the amplification products from normal control DNA and lane 2 contains the amplification products from tumor DNA. The bands result from 5′ host/virus junction fragments present in the DNA samples. Lane 1 has bands from the env/3′ LTR junctions from all proviruses (upper) and the host/5′ LTR from the pathogenic endogenous Mtv2 provirus present in this particular mouse strain. This endogenous provirus is detected because its sequence is identical to the new clonally integrated proviruses in the tumor. All four new clonally integrated proviruses known to be in this tumor are readily detected.

### Example 2

### Analysis of Quantitative RT-PCR: Comparative C_{T} Method

The expression level of target genes is quantified using the ABI PRISM 7900HT Sequence Detection System (Applied Biosystems, California). The method is based on the quantitation of the initial copy number of target template in comparison to that of a reference (normalizer) housekeeper gene (Pre-Developed TaqMan® Assay Reagents Gene Expression Quantification Protocol, Applied Biosystems, 2001). Accumulation of DNA product with each PCR cycle is related to amplicon efficiency and the initial template concentration. Therefore the amplification efficiency of both the target and the normalizer must be approximately equal. The threshold cycle (C_{T}), which is dependent on the starting template copy number and the DNA amplification efficiency, is a PCR cycle during which PCR product growth is exponential. With a similar dynamic range for the target and normalizer, the comparative C_{T} method is applicable.

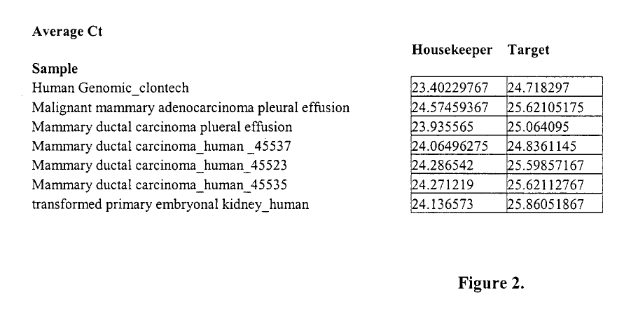

An example of the comparative C_{T} method of gene expression for quantitative RT-PCR is shown in FIG. 2. In the first step, assays are performed in quadruplicate on a normal tissue and several sample tissues. In these tissues, the means and standard deviations of C_{T} values are determined for housekeeper genes (chosen as controls if shown to be biologically stable among various samples, irrespective of disease state) and for the target gene. FIG. 2 shows an example of average C_{T} values for a housekeeper gene and target gene. These values can fall within a range from upper teens to 40 depending on the intrinsic expression level of the gene in the particular tissue. The coefficient of variance of all replicate sets cannot exceed 1.5%.

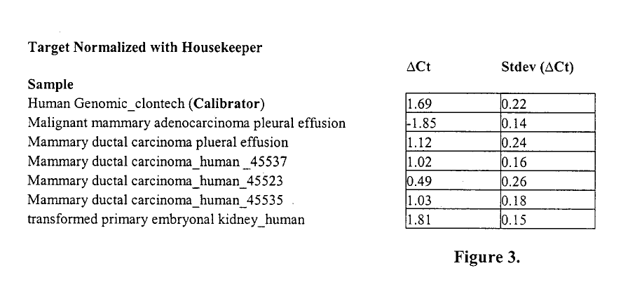

An assessment of how the ΔC_{T} changes with template dilution verifies that the efficiencies of the target and housekeeper amplicons are approximately equal if the log input amount of template RNA versus ΔC_{T} plot has a slope <0.10. With the relative efficiencies verified for target and housekeeper, the ΔΔC_{T} comparative calculation becomes valid, as mentioned above. An example of the calculated difference between the C_{T} values of target and housekeeper genes (ΔC_{T}) for various samples is shown in FIG. 3. The ΔΔC_{T} is calculated for each sample by subtracting its ΔC_{T} value from the ΔC_{T} value of the baseline (calibrator) sample. If the expression is increased in some samples and decreased in others, ΔΔC_{T} will be a mixture of negative and positive values. The final step in the calculation is to transform these values to absolute values. The formula for this is:

Comparative expression level=2^{-ΔΔCT}

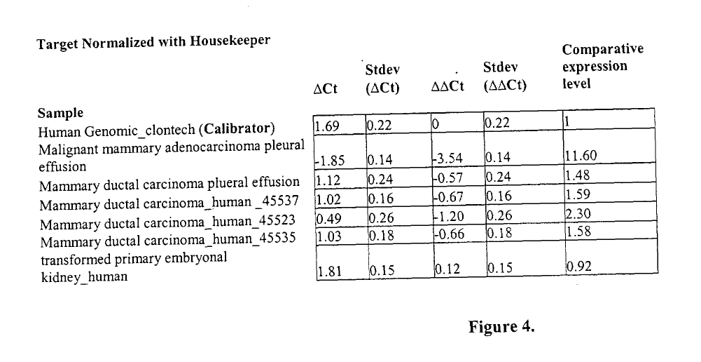

The final value for the calibrator should always be one. FIG. 4 shows the ΔΔC_{T} and comparative expression level for each sample from FIG. 3.

### Example 3

### Analysis of mRNA Expression of Various Genes in Cancer Samples

mRNA was prepared from various cancer samples as by standard procedures as are known in the art. Gene expression was measured as described in Example 2 above and as further described below. The 5′-nuclease (TaqMan) chemistry differs from standard PCR by the addition of a dual-labeled (reporter and quencher) fluorescent probe which anneals between the two PCR primers. The fluorescence of the reporter dye is quenched by the quencher being in close proximity. During thermal cycling, the 5′ nuclease activity of Taq DNA polymerase cleaves the annealed probe and liberates the reporter and quencher dyes. An increase in fluorescence is seen, and the cycle number in which the fluorescence increases above background is related to the starting template concentration in a log-linear fashion.

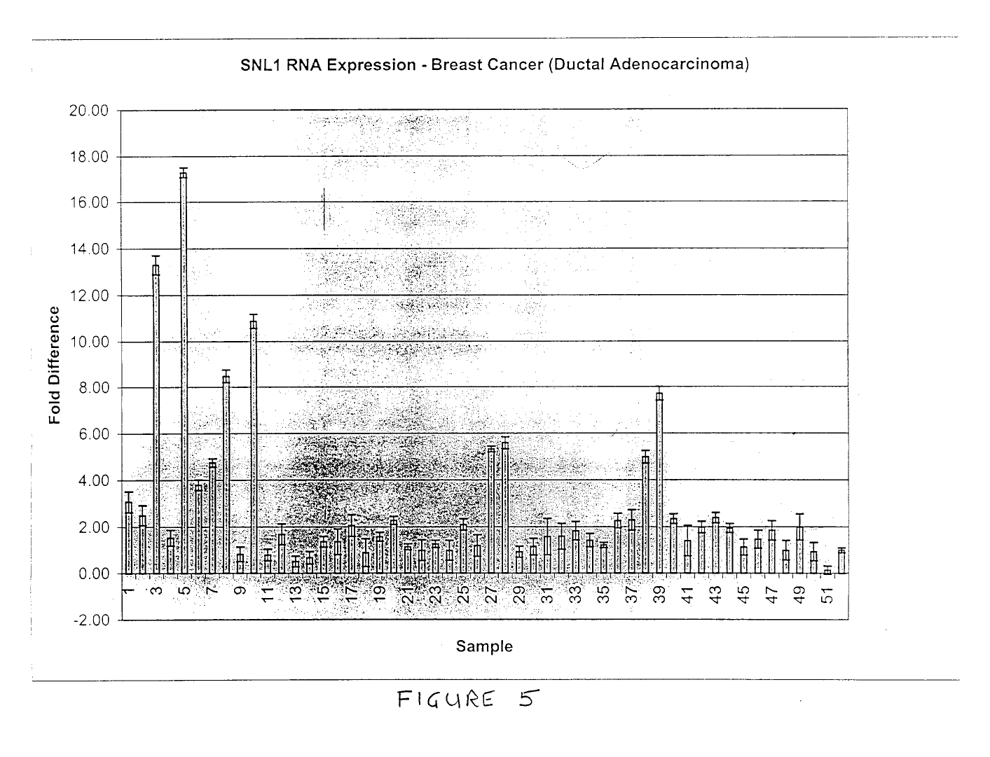

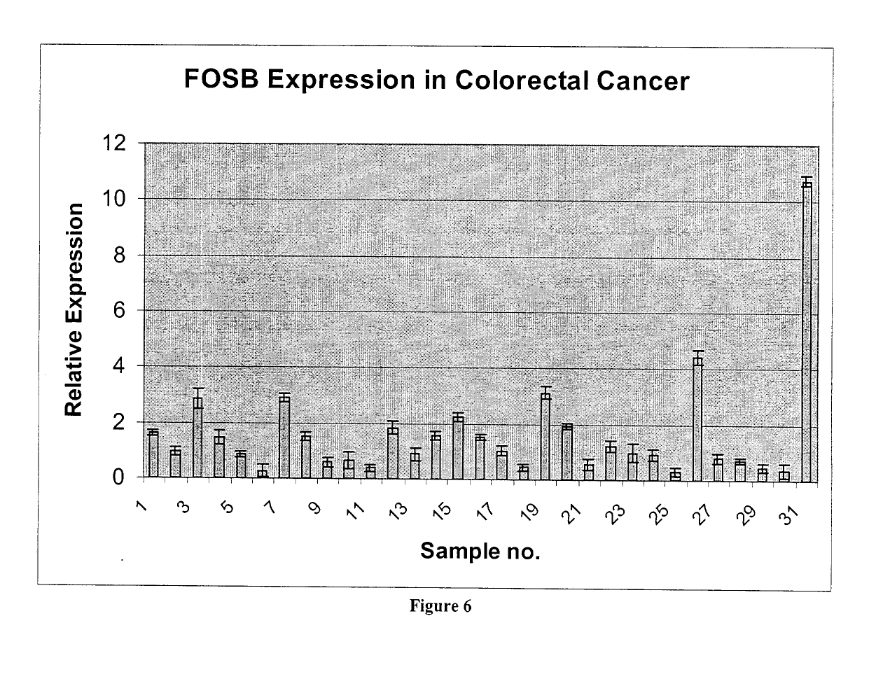

For data analysis, expression level of the target gene was normalized with the expression level of a house keeping gene. The mean level of expression of the housekeeping gene was subtracted from the mean expression level of the target gene. Standard deviation was then determined. In addition, the expression level of the target gene in cancer tissue is compared with the expression level of the target gene in normal tissue. In FIGS. 5-24, bars represent the mean of expression level and error bars represent standard deviation.

(i) SNL1

FIG. 5 depicts mRNA expression of SNL1 in breast cancer tissue compared with expression in normal tissue. Samples 1-50 are breast cancer samples. Samples 51 and 52 are normal tissues. SNL1 was up-regulated in approximately 20% of breast cancer samples examined.

(ii) FOSB

FIG. 6 depicts mRNA expression of FOSB in colon cancer tissue compared with expression in normal tissue. Samples 1-11 are normal samples. Samples 12-31 are colon cancer tissues.

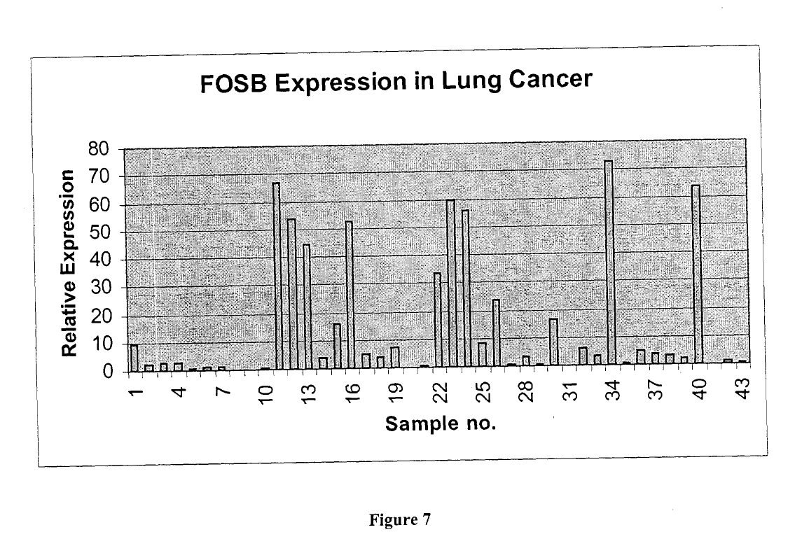

FIG. 7 depicts mRNA expression of FOSB in lung cancer tissue compared with expression in normal tissue. Samples 1-9 are normal samples. Samples 10-43 are lung cancer tissues.

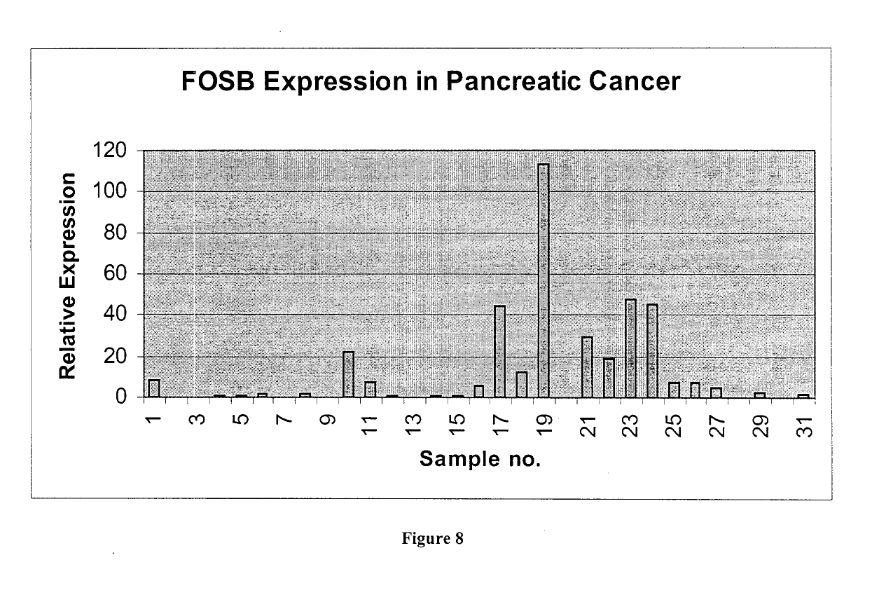

FIG. 8 depicts mRNA expression of FOSB in pancreas cancer tissue compared with expression in normal tissue. Samples 1-10 are normal samples. Samples 11-31 are pancreas cancer tissues.

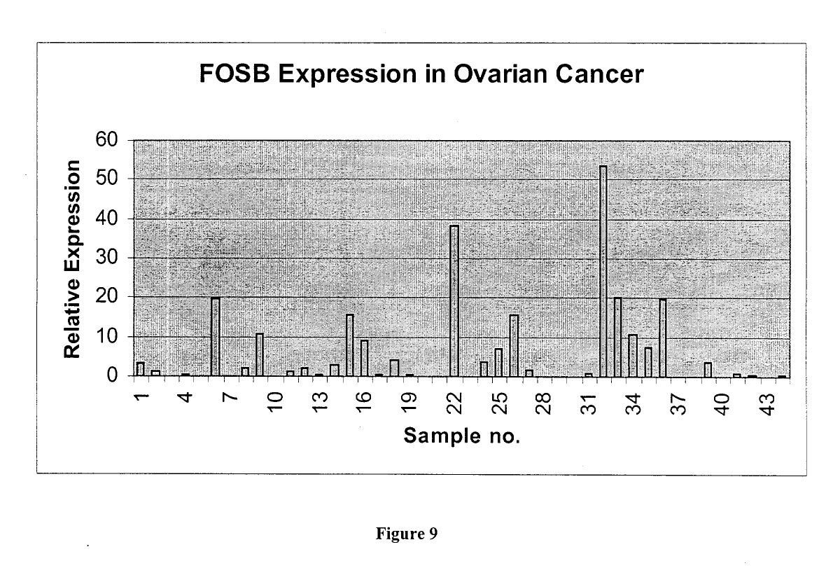

FIG. 9 depicts mRNA expression of FOSB in ovary cancer tissue compared with expression in normal tissue. Samples 1-16 are normal samples. Samples 17-44-are ovary cancer tissues.

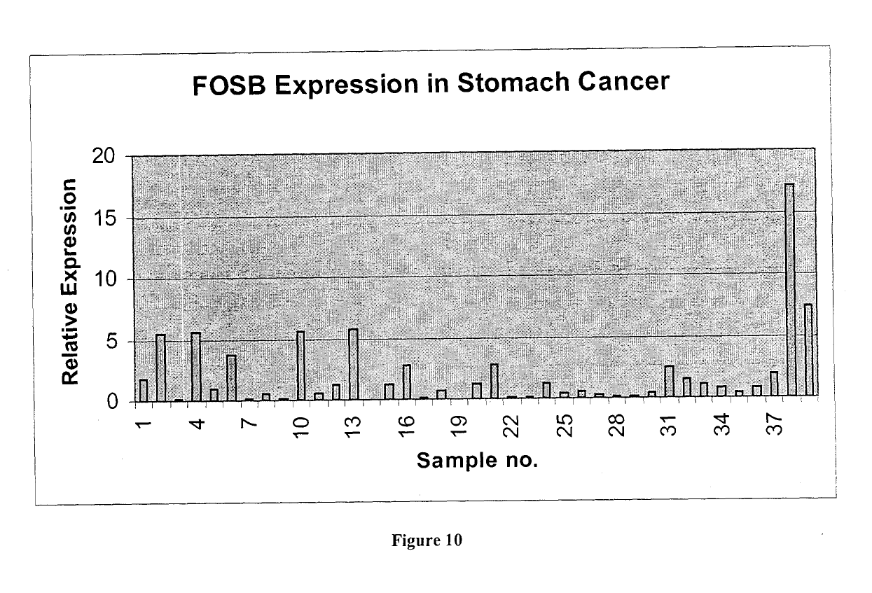

FIG. 10 depicts mRNA expression of FOSB in stomach cancer tissue compared with expression in normal tissue. Samples 1-10 are normal samples. Samples 11-39 are stomach cancer tissues.

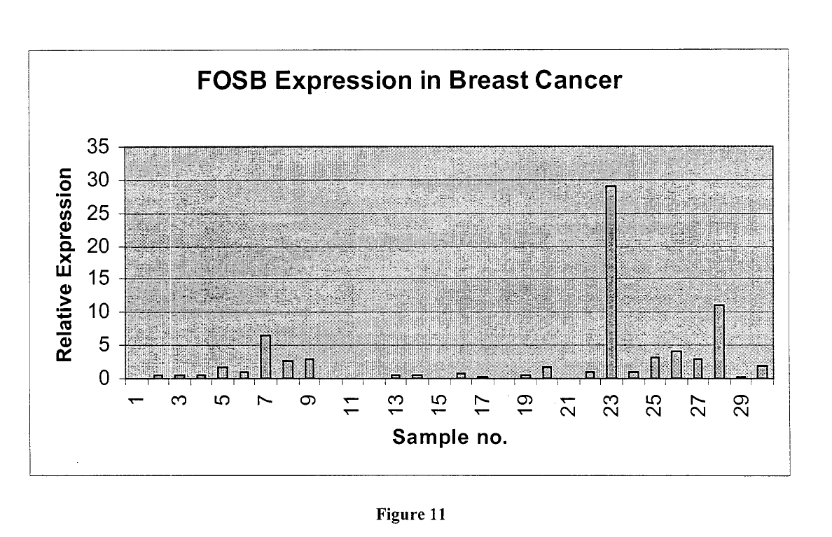

FIG. 11 depicts mRNA expression of FOSB in breast cancer tissue compared with expression in normal tissue. Samples 1-9 are normal samples. Samples 10-30 are breast cancer tissues. FOSB was up-regulated in approximately 26% of breast cancer samples examined.

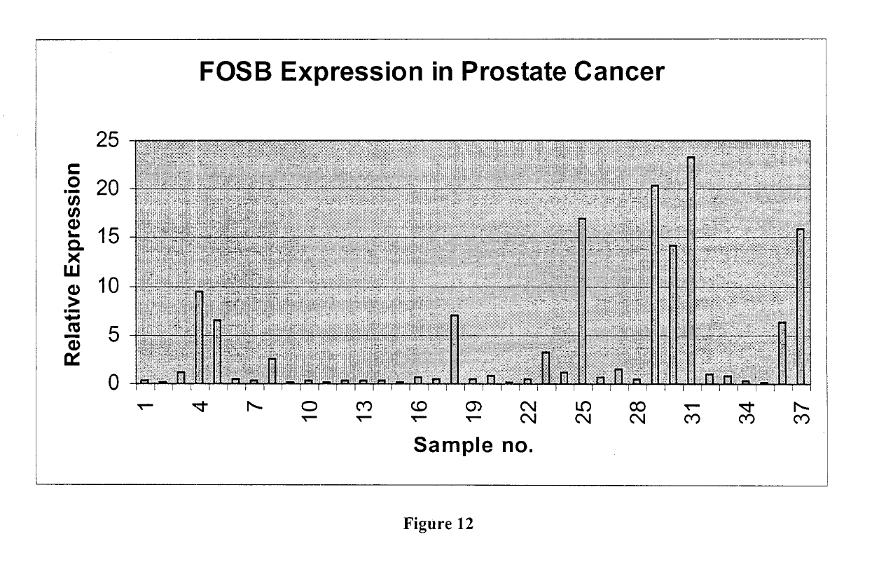

FIG. 12 depicts mRNA expression of FOSB in prostate cancer tissue compared with expression in normal tissue. Samples 1-7 are normal samples. Samples 8-37 are prostate cancer tissues.

(iii) MYC

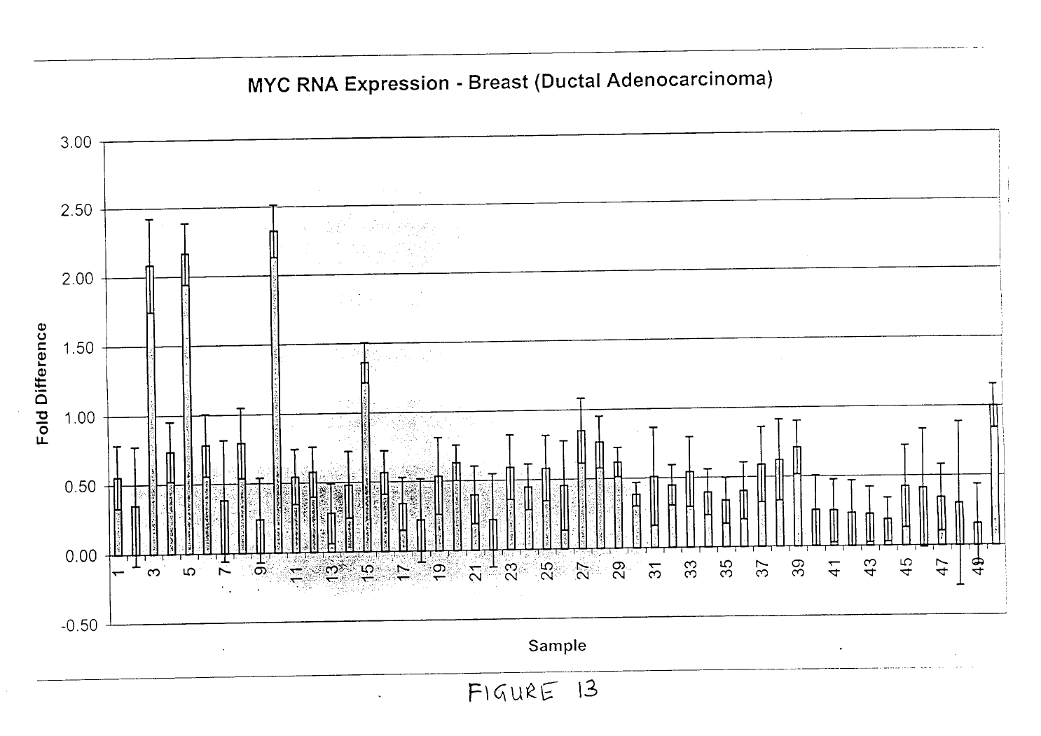

FIG. 13 depicts mRNA expression of MYC in breast cancer tissue compared with expression in normal tissue. Samples 1-50 are breast cancer samples. MYC was up-regulated in approximately 8% of breast cancer samples examined.

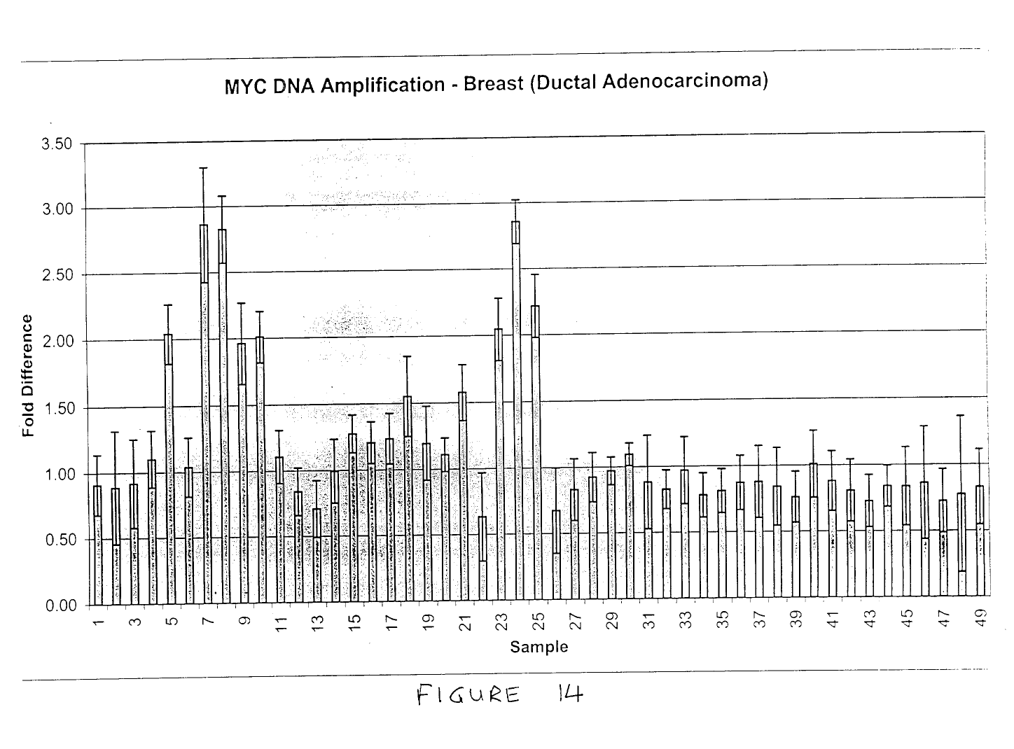

FIG. 14 depicts DNA amplification of MYC in breast cancer tissue compared with expression in normal tissue. Samples 1-49 are breast cancer samples. MYC DNA was amplified in approximately 21% of breast cancer samples examined.

(iv) CCND1

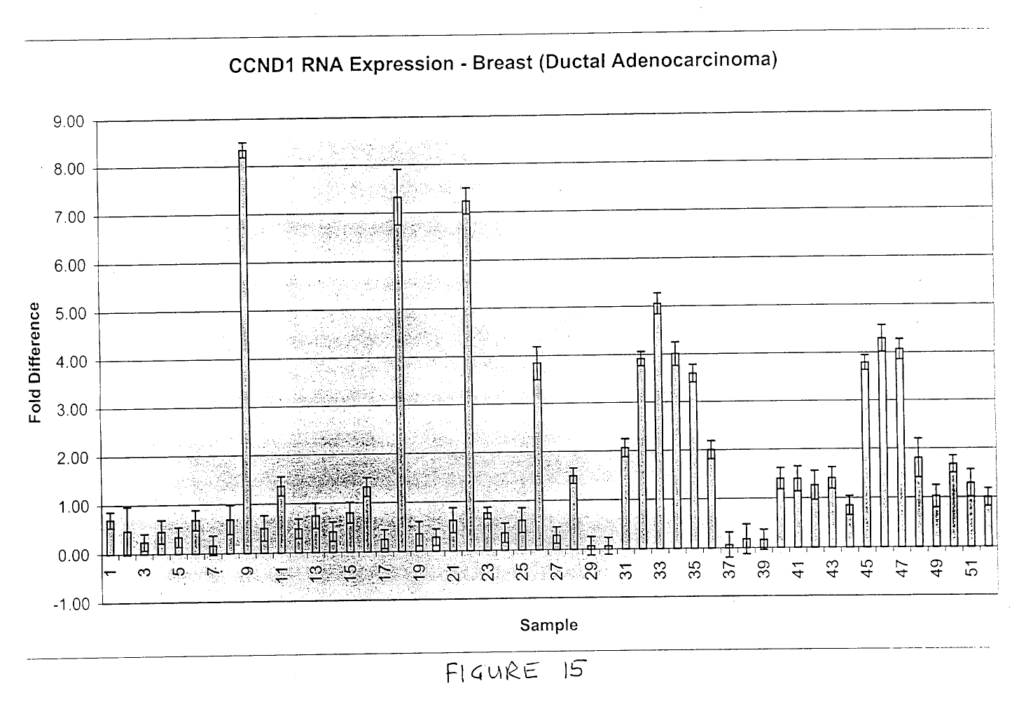

FIG. 15 depicts mRNA expression of CCND1 in breast cancer tissue compared with expression in normal tissue. Samples 1-50 are breast cancer samples. Samples 51 and 52 are normal tissues. CCND1 was up-regulated in approximately 26% of breast cancer samples examined.

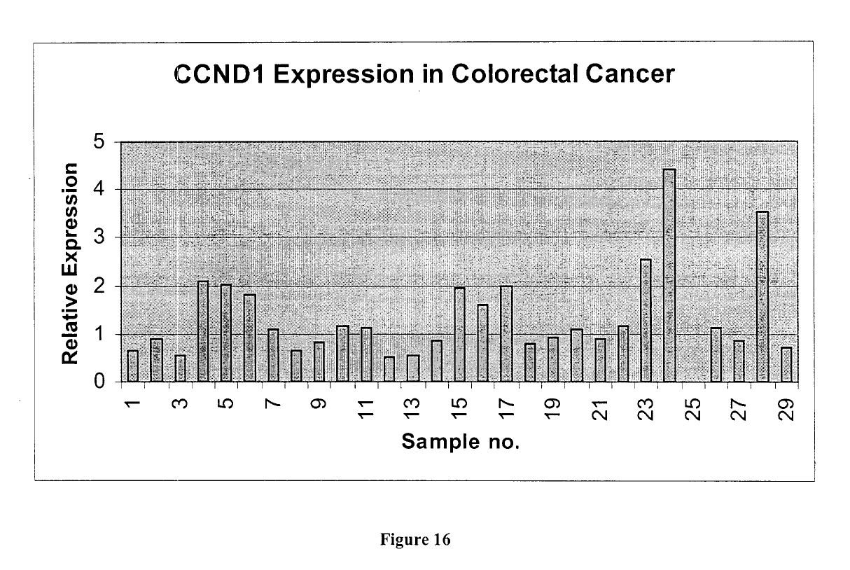

FIG. 16 depicts mRNA expression of CCND1 in colon cancer (sigmoid) tissue compared with expression in normal tissue. Samples 1-12 are normal samples. Samples 13-29 are colon cancer tissues.

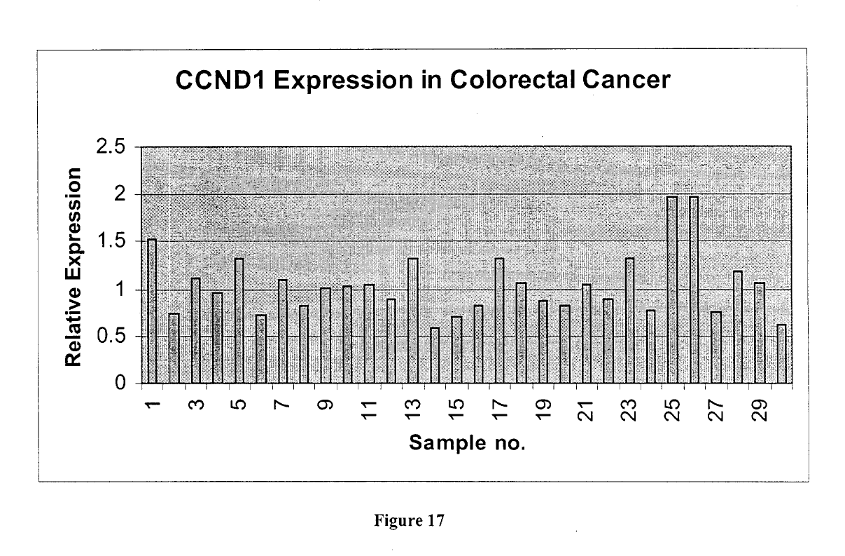

FIG. 17 depicts mRNA expression of CCND1 in colon cancer (transverse) tissue compared with expression in normal tissue. Samples 1-12 are normal samples. Samples 13-30 are colon cancer tissues.

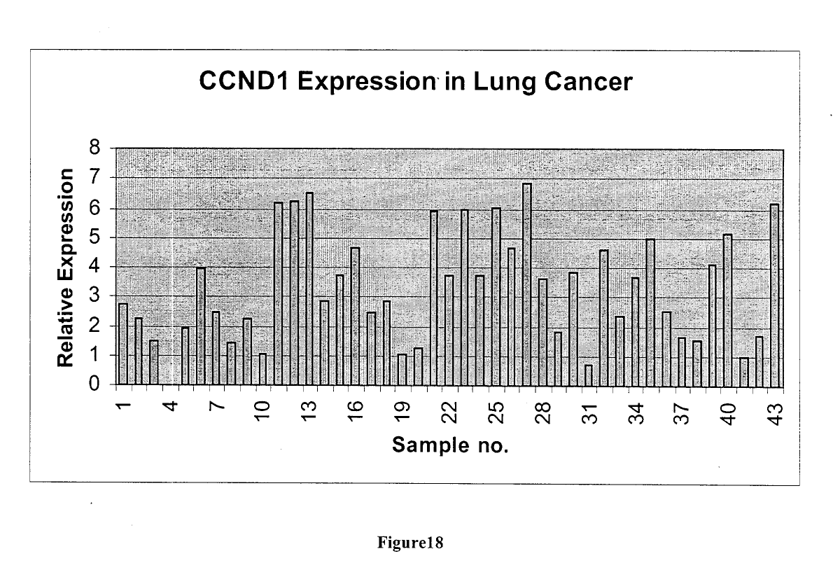

FIG. 18 depicts mRNA expression of CCND1 in lung tissue compared with expression in normal tissue. Samples 1-9 are normal samples. Samples 10-43 are lung cancer tissues.

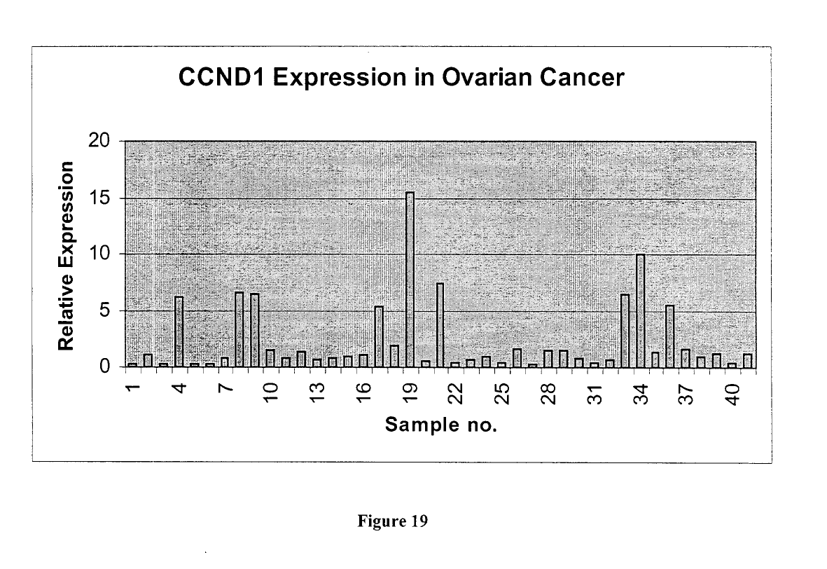

FIG. 19 depicts mRNA expression of CCND1 in ovary tissue compared with expression in normal tissue. Samples 1-14 are normal samples. Samples 15-41 are ovary cancer tissues.

(v) NFKB1

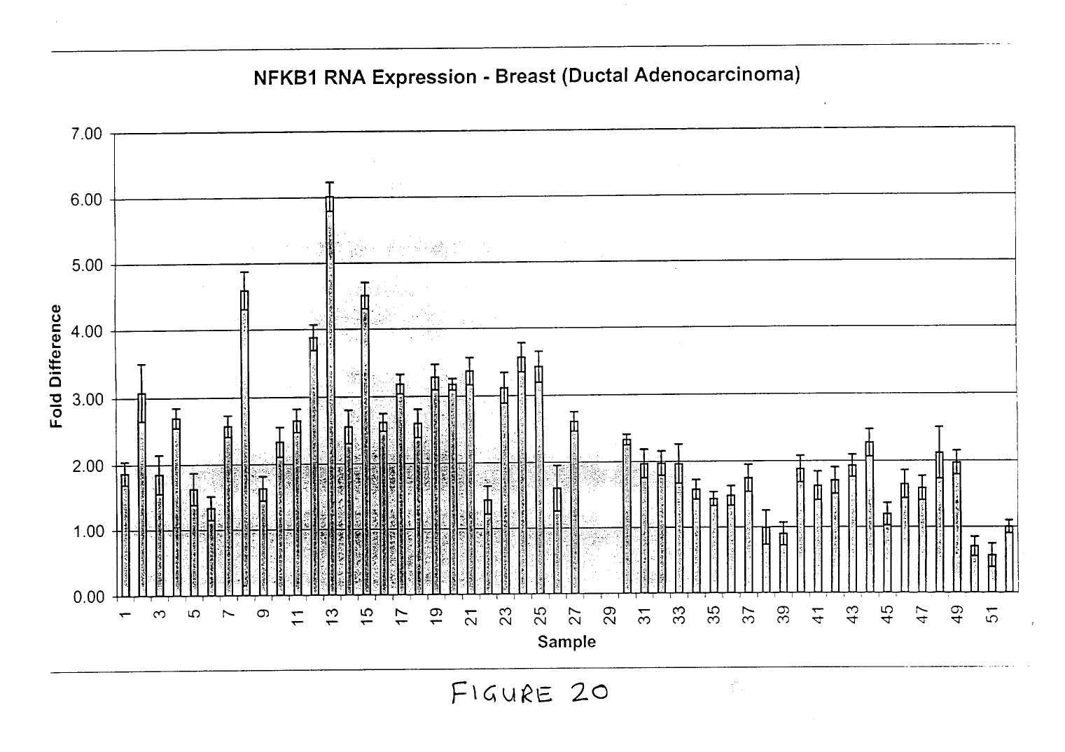

FIG. 20 depicts mRNA expression of NFKB1 in breast cancer tissue compared with expression in normal tissue. Samples 1-50 are breast cancer samples. Samples 51 and 52 are normal tissues. NFKB1 was up-regulated in approximately 25% of breast cancer samples examined.

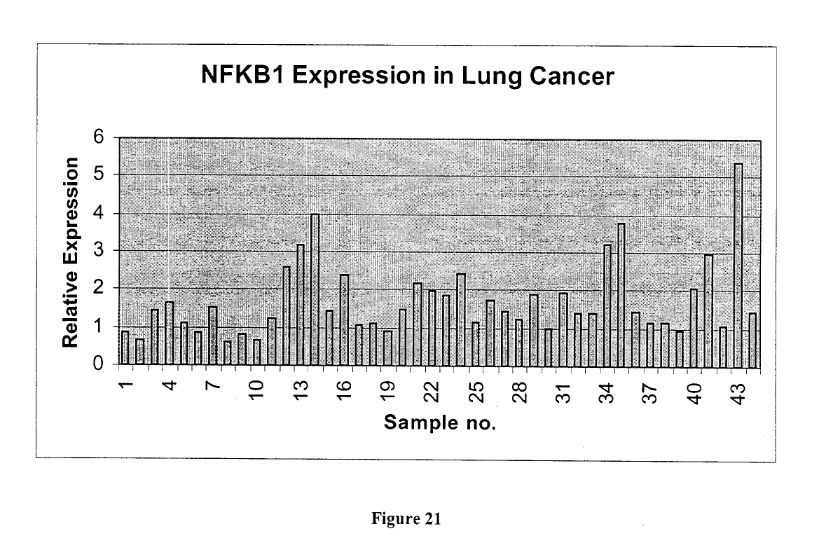

FIG. 21 depicts mRNA expression of NFKB1 in lung tissue compared with expression in normal tissue. Samples 1-9 are normal samples. Samples 10-44 are lung cancer tissues.

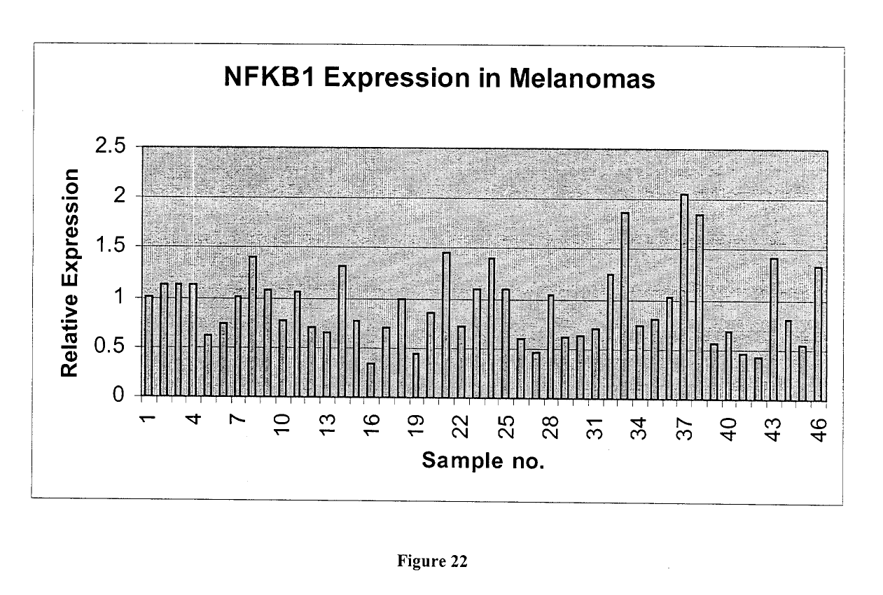

FIG. 22 depicts mRNA expression of NFKB1 in skin tissue compared with expression in normal tissue. Samples 1-9 are normal samples. Samples 10-46 are skin cancer tissues.

(vi) PVT1

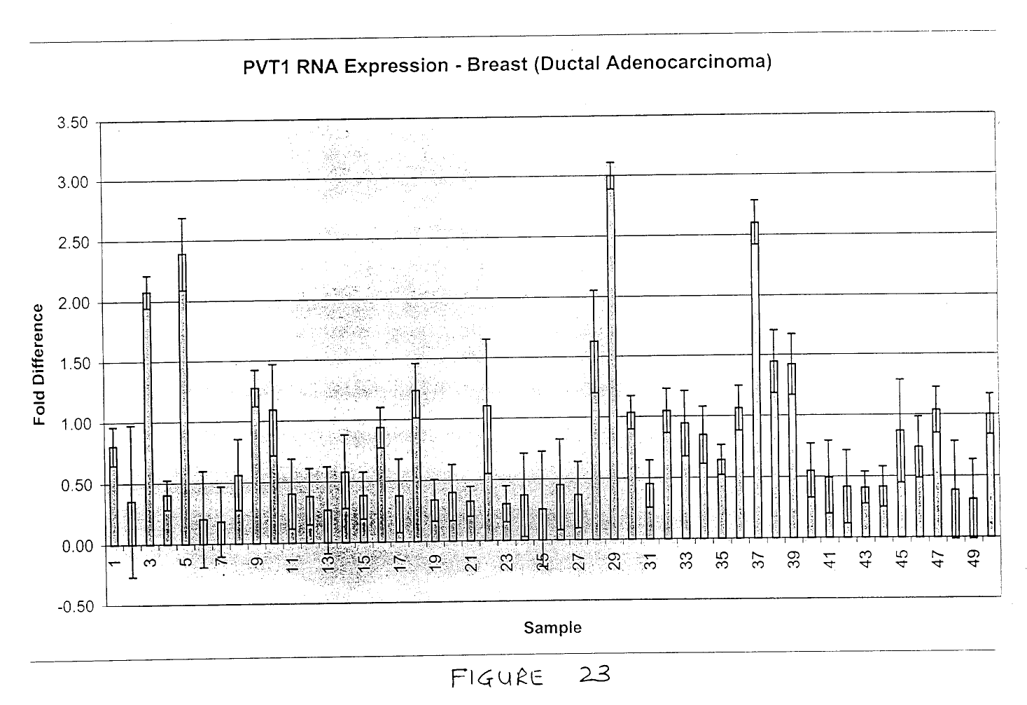

FIG. 23 depicts mRNA expression of PVT1 in breast cancer tissue compared with expression in normal tissue. Samples 1-50 are breast cancer samples. PVT1 was up-regulated in approximately 10% of breast cancer samples examined.

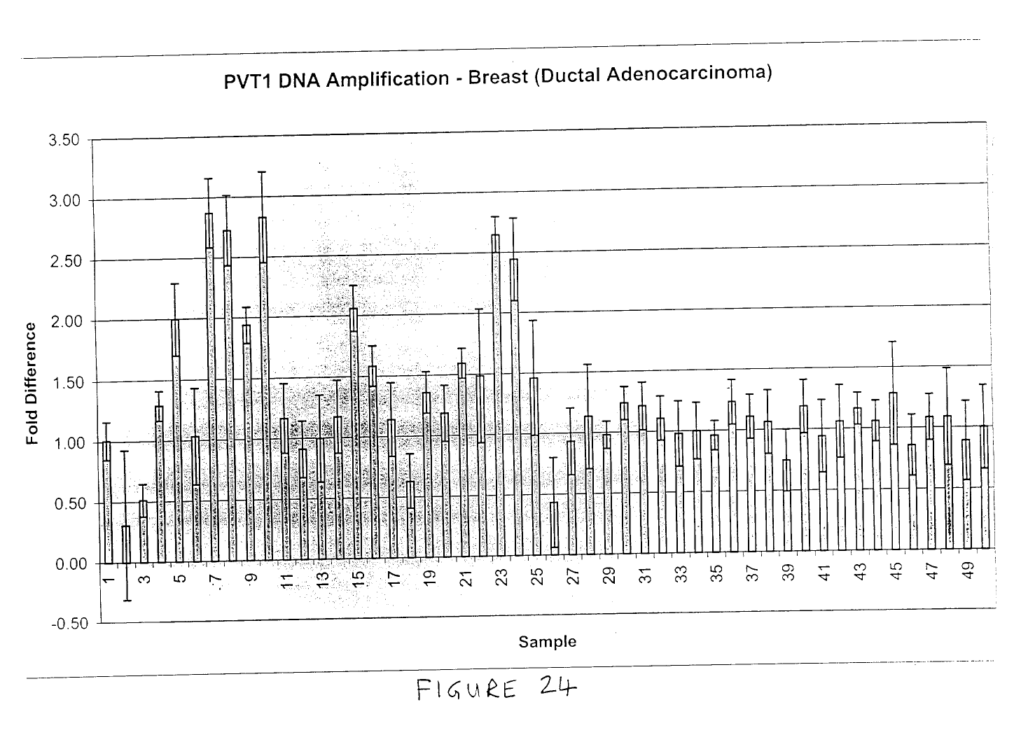

FIG. 24 depicts DNA amplification of PVT1 in breast cancer tissue compared with expression in normal tissue. Samples 1-50 are breast cancer samples.

### Example 4

### Detection of Elevated Levels of cDNA Associated with Cancer Using Arrays

cDNA sequences representing a variety of candidate CA genes to be screened for differential expression in cancer are assayed by hybridization on polynucleotide arrays. The cDNA sequences include cDNA clones isolated from cell lines or tissues of interest. The cDNA sequences analyzed also include polynucleotides comprising sequence overlap with sequences in the Unigene database, and which encode a variety of gene products of various origins, functionality, and levels of characterization. cDNAs are spotted onto reflective slides (Amersham) according to methods well known in the art at a density of 9,216 spots per slide representing 4,068 sequences (including controls) spotted in duplicate, with approximately 0.8 μl of an approximately 200 ng/μl solution of cDNA.

PCR products of selected cDNA clones corresponding to the gene products of interest are prepared in a 50% DMSO solution. These PCR products are spotted onto Amersham aluminum microarray slides at a density of 9216 clones per array using a Molecular Dynamics Generation III spotting robot. Clones are spotted in duplicate, for a total of 4608 different sequences per chip.

cDNA probes are prepared from total RNA obtained by laser capture microdissection (LCM, Arcturus Enginering Inc., Mountain View, Calif.) of tumor tissue samples and normal tissue samples isolated from patients.

Total RNA is first reverse transcribed into cDNA using a primer containing a T7 RNA polymerase promoter, followed by second strand DNA synthesis. cDNA is then transcribed in vitro to produce antisense RNA using the T7 promoter-mediated expression (see, e.g., Luo et al (1999) Nature Med 5:117-122), and the antisense RNA is then converted into cDNA. The second set of cDNAs are again transcribed in vitro, using the T7 promoter, to provide antisense RNA. This antisense RNA is then fluorescently labeled, or the RNA is again converted into cDNA, allowing for a third round of T7-mediated amplification to produce more antisense RNA. Thus the procedure provides for two or three rounds of in vitro transcription to produce the final RNA used for fluorescent labeling. Probes are labeled by making fluorescently labeled cDNA from the RNA starting material. Fluorescently labeled cDNAs prepared from the tumor RNA sample are compared to fluorescently labeled cDNAs prepared from normal cell RNA sample. For example, the cDNA probes from the normal cells are labeled with Cy3 fluorescent dye (green) and the cDNA probes prepared from suspected cancer cells are labeled with Cy5 fluorescent dye (red).

The differential expression assay is performed by mixing equal amounts of probes from tumor cells and normal cells of the same patient. The arrays are prehybridized by incubation for about 2 hrs at 60° C. in 5×SSC, 0.2% SDS, 1 mM EDTA, and then washing three times in water and twice in isopropanol. Following prehybridization of the array, the probe mixture is then hybridized to the array under conditions of high stringency (overnight at 42° C. in 50% formamide, 5×SSC, and 0.2% SDS. After hybridization, the array is washed at 55° C. three times as follows: 1) first wash in 1×SSC/0.2% SDS; 2) second wash in 0.1×SSC/0.2% SDS; and 3) third wash in 0.1×SSC.

The arrays are then scanned for green and red fluorescence using a Molecular Dynamics Generation III dual color laser-scanner/detector. The images are processed using BioDiscovery Autogene software, and the data from each scan set normalized. The experiment is repeated, this time labeling the two probes with the opposite color in order to perform the assay in both “color directions.” Each experiment is sometimes repeated with two more slides (one in each color direction). The data from each scan is normalized, and the level of fluorescence for each sequence on the array expressed as a ratio of the geometric mean of 8 replicate spots/genes from the four arrays or 4 replicate spots/gene from 2 arrays or some other permutation.

Normalization: The objective of normalization is to generate a cDNA library in which all transcripts expressed in a particular cell type or tissue are equally represented (S. M. Weissman, Mol Biol. Med. 4(3):133-143 (1987); Patanjali, et al., Proc. Natl. Acad. Sci. USA 88(5):1943-1947 (1991)), and therefore isolation of as few as 30,000 recombinant clones in an optimally normalized library may represent the entire gene expression repertoire of a cell, estimated to number 10,000 per cell.

Total RNA is extracted from harvested cells using RNeasy™ Protect Kit (Qiagen, Valencia, Calif.), following manufacturer's recommended procedures. RNA is quantified using RiboGreen™ RNA quantification kit (Molecular Probes, Inc. Eugene, Oreg.). One fig of total RNA is reverse transcribed and PCR amplified using SMART™ PCR cDNA synthesis kit (ClonTech, Palo Alto, Calif.). The cDNA products are size-selected by agarose gel electrophoresis using standard procedures (Sambrook, J. T., et al. Molecular Cloning: A Laboratory Manual, 2d ed., Cold Spring Harbor Laboratory Press, NY). The cDNA is extracted using Bio 101 Geneclean® II kit (Qbiogene, Carlsbad, Calif.). Normalization of the cDNA is carried out using kinetics of hybridization principles: 1.0 μg of cDNA is denatured by heat at 100° C. for 10 minutes, then incubated at 42° C. for 42 hours in the presence of 120 mM NaCl, 10 mM Tris.HCl (pH=8.0), 5 mM EDTA.Na+ and 50% formamide. Single-stranded cDNA (“normalized”) is purified by hydroxyapatite chromatography (#130-0520, BioRad, Hercules, Calif.) following the manufacturer's recommended procedures, amplified and converted to double-stranded cDNA by three cycles of PCR amplification, and cloned into plasmid vectors using standard procedures (Sambrook, J. T., et al. Molecular Cloning: A Laboratory Manual, 2d ed., Cold Spring Harbor Laboratory Press, NY). All primers/adaptors used in the normalization and cloning process are provided by the manufacturer in the SMART™ PCR cDNA synthesis kit (ClonTech, Palo Alto, Calif.). Supercompetent cells (XL-2 Blue Ultracompetent Cells, Stratagene, California) are transfected with the normalized cDNA libraries, plated on solid media and grown overnight at 36° C.

The sequences of 10,000 recombinants per normalized library are analyzed by capillary sequencing using the ABI PRISM 3700 DNA Analyzer (Applied Biosystems, California). To determine the representation of transcripts in a library, BLAST analysis is performed on the clone sequences to assign transcript identity to each isolated clone, i.e., the sequences of the isolated polynucleotides are first masked to eliminate low complexity sequences using the XBLAST masking program (Claverie “Effective Large-Scale Sequence Similarity Searches,” Computer Methods for Macromolecular Sequence Analysis, Doolittle, ed., Meth. Enzymol. 266:212-227 Academic Press, NY, N.Y. (1996); see particularly Clayerie, in “Automated DNA Sequencing and Analysis Techniques” Adams et al., eds., Chap. 36, p. 267 Academic Press, San Diego, 1994 and Clayerie et al. Comput. Chem. (1993) 17:191). Generally, masking does not influence the final search results, except to eliminate sequences of relative little interest due to their low complexity, and to eliminate multiple “hits” based on similarity to repetitive regions common to multiple sequences, e.g., Alu repeats. The remaining sequences are then used in a BLASTN vs. GenBank search. The sequences are also used as query sequence in a BLASTX vs. NRP (non-redundant proteins) database search.

Automated sequencing reactions are performed using a Perkin-Elmer PRISM Dye Terminator Cycle Sequencing Ready Reaction Kit containing AmpliTaq DNA Polymerase, FS, according to the manufacturer's directions. The reactions are cycled on a GeneAmp PCR System 9600 as per manufacturer's instructions, except that they are annealed at 20° C. or 30° C. for one minute. Sequencing reactions are ethanol precipitated, pellets are resuspended in 8 microliters of loading buffer, 1.5 microliters is loaded on a sequencing gel, and the data is collected by an ABI PRISM 3700 DNA Sequencer. (Applied Biosystems, Foster City, Calif.).

The number of times a sequence is represented in a library is determined by performing sequence identity analysis on the cloned cDNA sequences and assigning transcript identity to each isolated clone. First, each sequence is checked to determine if it is a bacterial, ribosomal, or mitochondrial contaminant. Such sequences are excluded from the subsequent analysis. Second, sequence artifacts, such as vector and repetitive elements, are masked and/or removed from each sequence.

The remaining sequences are compared via BLAST (Altschul et. al, J. Mol. Biol., 215:40, 1990) to GenBank and EST databases for gene identification and are compared with each other via FastA (Pearson & Lipman, PNAS, 85:2444, 1988) to calculate the frequency of cDNA appearance in the normalized cDNA library. The sequences are also searched against the GenBank and GeneSeq nucleotide databases using the BLASTN program (BLASTN 1.3MP: Altschul et al., J. Mol. Bio. 215:403, 1990). Fourth, the sequences are analyzed against a non-redundant protein (NRP) database with the BLASTX program (BLASTX 1.3MP: Altschul et al., supra). This protein database is a combination of the Swiss-Prot, PIR, and NCBI GenPept protein databases. The BLASTX program is run using the default BLOSUM-62 substitution matrix with the filter parameter: “xnu+seg”. The score cutoff utilized is 75. Assembly of overlapping clones into contigs is done using the program Sequencher (Gene Codes Corp.; Ann Arbor, Mich.). The assembled contigs are analyzed using the programs in the GCG package (Genetic. Computer Group, University Research Park, 575 Science Drive, Madison, Wis. 53711) Suite Version 10.1.

### Example 5

### Detection of CA-Sequences in Human Cancer Cells and Tissues

DNA from prostate and breast cancer tissues and other human cancer tissues, human colon, normal human tissues including non-cancerous prostate, and from other human cell lines are extracted following the procedure of Delli Bovi et al. (1986, Cancer Res. 46:6333-6338). The DNA is resuspended in a solution containing 0.05 M Tris HCl buffer, pH 7.8, and 0.1 mM EDTA, and the amount of DNA recovered is determined by microfluorometry using Hoechst 33258 dye. Cesarone, C., et al., Anal Biochem 100:188-197 (1979).

Polymerase chain reaction (PCR) is performed using Taq polymerase following the conditions recommended by the manufacturer (Perkin Elmer Cetus) with regard to buffer, Mg²⁺, and nucleotide concentrations. Thermocycling is performed in a DNA cycler by denaturation at 94° C. for 3 min. followed by either 35 or 50 cycles of 94° C. for 1.5 min., 50° C. for 2 min. and 72° C. for 3 min. The ability of the PCR to amplify the selected regions of the CA gene is tested by using a cloned CA polynucleotide(s) as a positive template(s). Optimal Mg²⁺, primer concentrations and requirements for the different cycling temperatures are determined with these templates. The master mix recommended by the manufacturer is used. To detect possible contamination of the master mix components, reactions without template are routinely tested.

Southern blotting and hybridization are performed as described by Southern, E. M., (J. Mol. Biol. 98:503-517, 1975), using the cloned sequences labeled by the random primer procedure (Feinberg, A. P., et al., 1983, Anal. Biochem. 132:6-13). Prehybridization and hybridization are performed in a solution containing 6×SSPE, 5% Denhardt's, 0.5% SDS, 50% formamide, 100 μg/ml denaturated salmon testis DNA, incubated for 18 hrs at 420 C., followed by washings with 2×SSC and 0.5% SDS at room temperature and at 37° C. and finally in 0.1×SSC with 0.5% SDS at 68° C. for 30 min (Sambrook et al., 1989, in “Molecular Cloning: A Laboratory Manual”, Cold Spring Harbor Lab. Press). For paraffin-embedded tissue sections the conditions described by Wright and Manos (1990, in “PCR Protocols”, Innis et al., eds., Academic Press, pp. 153-158) are followed using primers designed to detect a 250 bp sequence.

### Example 6

### Detection of CA Sequences in Human Cancer Cells and Tissues

DNA from human cancer tissues, normal human tissues and from other human cell lines is extracted following the procedure of Delli Bovi et al. (1986, Cancer Res. 46:6333-6338). The DNA is resuspended in a solution containing 0.05 M Tris HCl buffer, pH 7.8, and 0.1 mM EDTA, and the amount of DNA recovered is determined by microfluorometry using Hoechst 33258 dye. Cesarone, C. et al., Anal Biochem 100:188-197 (1979).

Polymerase chain reaction (PCR) is performed using Taq polymerase following the conditions recommended by the manufacturer (Perkin Elmer Cetus) with regard to buffer, Mg²⁺, and nucleotide concentrations. Thermocycling is performed in a DNA cycler by denaturation at 94° C. for 3 min. followed by either 35 or 50 cycles of 94° C. for 1.5 min., 50° C. for 2 min. and 72° C. for 3 min. The ability of the PCR to amplify the selected regions of CA genes is tested by using a cloned CA polynucleotide(s) as a positive template(s). Optimal Mg²⁺, primer concentrations and requirements for the different cycling temperatures are determined with these templates. The master mix recommended by the manufacturer is used. To detect possible contamination of the master mix components, reactions without template are routinely tested.

Southern blotting and hybridization are performed as described by Southern, E. M., (J. Mol. Biol. 98:503-517, 1975), using the cloned sequences labeled by the random primer procedure (Feinberg, A. P., et al., 1983, Anal. Biochem. 132:6-13). Prehybridization and hybridization are performed in a solution containing 6×SSPE, 5% Denhardt's, 0.5% SDS, 50% formamide, 100 μg/ml denaturated salmon testis DNA, incubated for 18 hrs at 42° C., followed by washings with 2×SSC and 0.5% SDS at room temperature and at 37° C. and finally in 0.1×SSC with 0.5% SDS at 68° C. for 30 min (Sambrook et al., 1989, in “Molecular Cloning: A Laboratory Manual”, Cold Spring Harbor Lab. Press). For paraffin-embedded tissue sections the conditions described by Wright and Manos (1990, in “PCR Protocols”, Innis et al., eds., Academic Press, pp. 153-158) are followed using primers designed to detect a 250 bp sequence.

### Example 7

### Expression of Cloned Polynucleotides in Host Cells

To study the protein products of CA genes, restriction fragments from CA DNA are cloned into the expression vector pMT2 (Sambrook, et al., Molecular Cloning: A Laboratory Manual, Cold Spring Harbor Laboratory Press pp 16.17-16.22 (1989)) and transfected into COS cells grown in DMEM supplemented with 10% FCS. Transfections are performed employing calcium phosphate techniques (Sambrook, et al (1989) pp. 16.32-16.40, supra) and cell lysates are prepared forty-eight hours after transfection from both transfected and untransfected COS cells. Lysates are subjected to analysis by immunoblotting using anti-peptide antibody.

In immunoblotting experiments, preparation of cell lysates and electrophoresis are performed according to standard procedures. Protein concentration is determined using BioRad protein assay solutions. After semi-dry electrophoretic transfer to nitrocellulose, the membranes are blocked in 500 mM NaCl, 20 mM Tris, pH 7.5, 0.05% Tween-20 (TTBS) with 5% dry milk. After washing in TTBS and incubation with secondary antibodies (Amersham), enhanced chemiluminescence (ECL) protocols (Amersham) are performed as described by the manufacturer to facilitate detection.

### Example 8

### Generation of Antibodies Against Polypeptides

Polypeptides, unique to CA genes are synthesized or isolated from bacterial or other (e.g., yeast, baculovirus) expression systems and conjugated to rabbit serum albumin (RSA) with m-maleimido benzoic acid N-hydroxysuccinimide ester (MBS) (Pierce, Rockford, Ill.). Immunization protocols with these peptides are performed according to standard methods. Initially, a pre-bleed of the rabbits is performed prior to immunization. The first immunization includes Freund's complete adjuvant and 500 μg conjugated peptide or 100 μg purified peptide. All subsequent immunizations, performed four weeks after the previous injection, include Freund's incomplete adjuvant with the same amount of protein. Bleeds are conducted seven to ten days after the immunizations.

For affinity purification of the antibodies, the corresponding CA polypeptide is conjugated to RSA with MBS, and coupled to CNBr-activated Sepharose (Pharmacia, Uppsala, Sweden). Antiserum is diluted 10-fold in 10 mM Tris-HCl, pH 7.5, and incubated overnight with the affinity matrix. After washing, bound antibodies are eluted from the resin with 100 mM glycine, pH 2.5.

### Example 9

### Generation of Monoclonal Antibodies Against a CA Polypeptide

A non-denaturing adjuvant (Ribi, R730, Corixa, Hamilton Mont.) is rehydrated to 4 ml in phosphate buffered saline. 100 μl of this rehydrated adjuvant is then diluted with 400 μl of Hank's Balanced Salt Solution and this is then gently mixed with the cell pellet used for immunization. Approximately 500 μg conjugated peptide or 100 μg purified peptide and Freund's complete are injected into Balb/c mice via foot-pad, once a week. After 6 weeks of weekly injection, a drop of blood is drawn from the tail of each immunized animal to test the titer of antibodies against CA polypeptides using FACS analysis. When the titer reaches at least 1:2000, the mice are sacrificed in a CO₂ chamber followed by cervical dislocation. Lymph nodes are harvested for hybridoma preparation. Lymphocytes from mice with the highest titer are fused with the mouse myeloma line X63-Ag8.653 using 35% polyethylene glycol 4000. On day 10 following the fusion, the hybridoma supernatants are screened for the presence of CAP-specific monoclonal antibodies by fluorescence activated cell sorting (FACS). Conditioned medium from each hybridoma is incubated for 30 minutes with a combined aliquot of PC3, Colo-205, LnCap, or Panc-1 cells. After incubation, the cell samples are washed, resuspended in 0.1 ml diluent and incubated with 1 μg/ml of FITC conjugated F(ab′)2 fragment of goat anti-mouse IgG for 30 min at 4° C. The cells are washed, resuspended in 0.5 ml FACS diluent and analyzed using a FACScan cell analyzer (Becton Dickinson; San Jose, Calif.). Hybridoma clones are selected for further expansion, cloning, and characterization based on their binding to the surface of one or more of cell lines which express the CA polypeptide as assessed by FACS. A hybridoma making a monoclonal antibody designated mAbCA which binds an antigen designated Ag-CA.x and an epitope on that antigen designated Ag-CA.x.1 is selected.

### Example 10

### ELISA Assay for Detecting CA Related Antigens

To test blood samples for antibodies that bind specifically to recombinantly produced CA antigens, the following procedure is employed. After a recombinant CA related protein is purified, the recombinant protein is diluted in PBS to a concentration of 5 μg/ml (500 ng/100 μl). 100 microliters of the diluted antigen solution is added to each well of a 96-well Immulon 1 plate (Dynatech Laboratories, Chantilly, Va.), and the plate is then incubated for 1 hour at room temperature, or overnight at 4° C., and washed 3 times with 0.05% Tween 20 in PBS. Blocking to reduce nonspecific binding of antibodies is accomplished by adding to each well 200 μl of a 1% solution of bovine serum albumin in PBS/Tween 20 and incubation for 1 hour. After aspiration of the blocking solution, 100 μl of the primary antibody solution (anticoagulated whole blood, plasma, or serum), diluted in the range of 1/16 to 1/2048 in blocking solution, is added and incubated for 1 hour at room temperature or overnight at 4° C. The wells are then washed 3 times, and 100 μl of goat anti-human IgG antibody conjugated to horseradish peroxidase (Organon Teknika, Durham, N.C.), diluted 1/500 or 1/1000 in PBS/Tween 20, 100 μl of o-phenylenediamine dihydrochloride (OPD, Sigma) solution is added to each well and incubated for 5-15 minutes. The OPD solution is prepared by dissolving a 5 mg OPD tablet in 50 ml 1% methanol in H₂O and adding 50 μl 30% H₂O₂ immediately before use. The reaction is stopped by adding 25 l of 4M H₂SO₄. Absorbances are read at 490 nm in a microplate reader (Bio-Rad).

### Example 11

### Identification and Characterization of CA Antigen on Cancer Cell Surface

A cell pellet of proximately 25 ul packed cell volume of a cancer cell preparation is lysed by first diluting the cells to 0.5 ml in water followed by freezing and thawing three times. The solution is centrifuged at 14,000 rpm. The resulting pellet, containing the cell membrane fragments, is resuspended in 50 μl of SDS sample buffer (Invitrogen, Carlsbad, Calif.). The sample is heated at 80° C. for 5 minutes and then centrifuged for 2 minutes at 14,000 rpm to remove any insoluble materials.

The samples are analyzed by Western blot using a 4 to 20% polyacrylamide gradient gel in Tris-Glycine SDS (Invitrogen; Carlsbad Calif.) following the manufacturer's directions. Ten microliters of membrane sample are applied to one lane on the polyacrylamide gel. A separate 10 μL sample is reduced first by the addition of 2 μL of dithiothreitol (100 mM) with heating at 80° C. for 2 minutes and then loaded into another lane. Pre-stained molecular weight markers SeeBlue Plus2 (Invitrogen; Carlsbad, Calif.) are used to assess molecular weight on the gel. The gel proteins are transferred to a nitrocellulose membrane using a transfer buffer of 14.4 g/l glycine, 3 g/l of Tris Base, 10% methanol, and 0.05% SDS. The membranes are blocked, probed with a CAP-specific monoclonal antibody (at a concentration of 0.5 ug/ml), and developed using the Invitrogen WesternBreeze Chromogenic Kit-AntiMouse according to the manufacturer's directions. In the reduced sample of the tumor cell membrane samples, a prominent band is observed migrating at a molecular weight within about 10% of the predicted molecular weight of the corresponding CA protein.

### Example 12

### Preparation of Vaccines

The present invention also relates to a method of stimulating an immune response against cells that express CA polypeptides in a patient using CA polypeptides of the invention that act as an antigen produced by or associated with a malignant cell. This aspect of the invention provides a method of stimulating an immune response in a human against cancer cells or cells that express CA polynucleotides and polypeptides. The method comprises the step of administering to a human an immunogenic amount of a polypeptide comprising: (a) the amino acid sequence of a huma CA protein or (b) a mutein or variant of a polypeptide comprising the amino acid sequence of a human endogenous retrovirus CA protein.

### Example 13

### Generation of Transgenic Animals Expressing Polypeptides as a Means for Testing Therapeutics

CA nucleic acids are used to generate genetically modified non-human animals, or site specific gene modifications thereof, in cell lines, for the study of function or regulation of prostate tumor-related genes, or to create animal models of diseases, including prostate cancer. The term “transgenic” is intended to encompass genetically modified animals having an exogenous CA gene(s) that is stably transmitted in the host cells where the gene(s) may be altered in sequence to produce a modified protein, or having an exogenous CA LTR promoter operably linked to a reporter gene. Transgenic animals may be made through a nucleic acid construct randomly integrated into the genome. Vectors for stable integration include plasmids, retroviruses and other animal viruses, YACs, and the like. Of interest are transgenic mammals, e.g. cows, pigs, goats, horses, etc., and particularly rodents, e.g. rats, mice, etc.

The modified cells or animals are useful in the study of CA gene function and regulation. For example, a series of small deletions and/or substitutions may be made in the CA genes to determine the role of different genes in tumorigenesis. Specific constructs of interest include, but are not limited to, antisense constructs to block CA gene expression, expression of dominant negative CA gene mutations, and over-expression of a CA gene. Expression of a CA gene or variants thereof in cells or tissues where it is not normally expressed or at abnormal times of development is provided. In addition, by providing expression of proteins derived from CA in cells in which it is otherwise not normally produced, changes in cellular behavior can be induced.

DNA constructs for random integration need not include regions of homology to mediate recombination. Conveniently, markers for positive and negative selection are included. For various techniques for transfecting mammalian cells, see Keown et al., Methods in Enzymology 185:527-537 (1990).

For embryonic stem (ES) cells, an ES cell line is employed, or embryonic cells are obtained freshly from a host, e.g. mouse, rat, guinea pig, etc. Such cells are grown on an appropriate fibroblast-feeder layer or grown in the presence of appropriate growth factors, such as leukemia inhibiting factor (LIF). When ES cells are transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting chimeric animals screened for cells bearing the construct. By providing for a different phenotype of the blastocyst and the ES cells, chimeric progeny can be readily detected.

The chimeric animals are screened for the presence of the modified gene and males and females having the modification are mated to produce homozygous progeny. If the gene alterations cause lethality at some point in development, tissues or organs are maintained as allogeneic or congenic grafts or transplants, or in in vitro culture. The transgenic animals may be any non-human mammal, such as laboratory animals, domestic animals, etc. The transgenic animals are used in functional studies, drug screening, etc., e.g. to determine the effect of a candidate drug on prostate cancer, to test potential therapeutics or treatment regimens, etc.

### Example 14

### Diagnostic Imaging Using CA Specific Antibodies

The present invention encompasses the use of antibodies to CA polypeptides to accurately stage cancer patients at initial presentation and for early detection of metastatic spread of cancer. Radioimmunoscintigraphy using monoclonal antibodies specific for CA polypeptides can provide an additional cancer-specific diagnostic test. The monoclonal antibodies of the instant invention are used for histopathological diagnosis of carcinomas.

Subcutaneous human xenografts of cancer cells in nude mice is used to test whether a technetium-99m (^{99m}Tc)-labeled monoclonal antibody of the invention can successfully image the xenografted cancer by external gamma scintography as described for seminoma cells by Marks, et al., Brit. J. Urol. 75:225 (1995). Each monoclonal antibody specific for a CA polypeptide is purified from ascitic fluid of BALB/c mice bearing hybridoma tumors by affinity chromatography on protein A-Sepharose. Purified antibodies, including control monoclonal antibodies such as an avidin-specific monoclonal antibody (Skea, et al., J. Immunol. 151:3557 (1993)) are labeled with ^{99m}Tc following reduction, using the methods of Mather, et al., J. Nucl. Med. 31:692 (1990) and Zhang et al., Nucl. Med. Biol. 19:607 (1992). Nude mice bearing human cancer cells are injected intraperitoneally with 200-500 μCi of ^{99m}Tc-labeled antibody. Twenty-four hours after injection, images of the mice are obtained using a Siemens ZLC3700 gamma camera equipped with a 6 mm pinhole collimator set approximately 8 cm from the animal. To determine monoclonal antibody biodistribution following imaging, the normal organs and tumors are removed, weighed, and the radioactivity of the tissues and a sample of the injectate are measured. Additionally, CA-specific antibodies conjugated to antitumor compounds are used for cancer-specific chemotherapy.

### Example 15

### Immunohistochemical Methods

Frozen tissue samples from cancer patients are embedded in an optimum cutting temperature (OCT) compound and quick-frozen in isopentane with dry ice. Cryosections are cut with a Leica 3050 CM mictrotome at thickness of 5 μm and thaw-mounted on vectabound-coated slides. The sections are fixed with ethanol at −20° C. and allowed to air dry overnight at room temperature. The fixed sections are stored at −80° C. until use. For immunohistochemistry, the tissue sections are retrieved and first incubated in blocking buffer (PBS, 5% normal goat serum, 0.1% Tween 20) for 30 minutes at room temperature, and then incubated with the CA protein-specific monoclonal antibody and control monoclonal antibodies diluted in blocking buffer (1 μg/ml) for 120 minutes. The sections are then washed three times with the blocking buffer. The bound monoclonal antibodies are detected with a goat anti-mouse IgG+IgM (H+L) F(ab′)²-peroxidase conjugates and the peroxidase substrate diaminobenzidine (1 mg/ml, Sigma Catalog No. D 5637) in 0.1 M sodium acetate buffer pH 5.05 and 0.003% hydrogen peroxide (Sigma cat. No. H1009). The stained slides are counter-stained with hematoxylin and examined under Nikon microscope.

Monoclonal antibody against a CA protein (antigen) is used to test reactivity with various cell lines from different types of tissues. Cells from different established cell lines are removed from the growth surface without using proteases, packed and embedded in OCT compound. The cells are frozen and sectioned, then stained using a standard IHC protocol. The CellArray™ technology is described in WO 01/43869. Normal tissue (human) obtained by surgical resection are frozen and mounted. Cryosections are cut with a Leica 3050 CM mictrotome at thickness of 5 μm and thaw-mounted on vectabound-coated slides. The sections are fixed with ethanol at −20° C. and allowed to air dry overnight at room temperature. PolyMICA™ Detection kit is used to determine binding of a CA-specific monoclonal antibody to normal tissue. Primary monoclonal antibody is used at a final concentration of 1 μg/ml.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. An isolated nucleic acid comprising at least 10 contiguous nucleotides of a sequence selected from the group consisting of the human polynucleotide mRNA sequences of SEQ ID NOS: 5, 11, 17, 23, 29 and 32 shown in Tables 1-6, or its complement.

2. A host cell comprising a recombinant nucleic acid of claim 1.

3. An expression vector comprising the isolated nucleic acid according to claim 1.

4. A host cell comprising the expression vector of claim 3.

5. The polynucleotide according to claim 1, wherein said polynucleotide, or its complement or a fragment thereof, further comprises a detectable label.

6. The polynucleotide according to claim 1, wherein said polynucleotide, or its complement or a fragment thereof, is attached to a solid support.

7. The polynucleotide according to claim 1, wherein said polynucleotide, or its complement or a fragment thereof, is prepared at least in part by chemical synthesis.

8. The polynucleotide according to claim 1, wherein said polynucleotide, or its complement or a fragment thereof, is an antisense fragment.

9. The polynucleotide according to claim 1, wherein said polynucleotide, or its complement or a fragment thereof, is single stranded.

10. The polynucleotide according to claim 1, wherein said polynucleotide, or its complement or a fragment thereof, is double stranded.

11. The polynucleotide according to claim 1, comprising at least 15 contiguous nucleotides.

12. The polynucleotide according to claim 1, comprising at least 20 contiguous nucleotides.

13. A microarray for detecting a cancer associated (CA) nucleic acid comprising:
at least one probe comprising at least 10 contiguous nucleotides of a sequence selected from the group consisting of the human polynucleotide sequences of SEQ ID NOS: 5, 11, 17, 23, 29 and 32 shown in Tables 1-6, or its complement.

14. The microarray according to claim 13, comprising at least 15 contiguous nucleotides.

15. The microarray according to claim 13, comprising at least 20 contiguous nucleotides.

16. An isolated polypeptide, encoded within an open reading frame of a CA sequence selected from the group consisting of the human genomic polynucleotide sequences of SEQ ID NOS: 4, 10, 16, 22, 28 and 31 shown in Tables 1-6, or its complement.

17. The polypeptide of claim 16, wherein said polypeptide comprises the amino acid sequence encoded by a human polynucleotide selected from the group consisting of SEQ ID NOS: 5, 11, 17, 23, 29 and 32 shown in Tables 1-6.

18. The polypeptide of claim 16, wherein said polypeptide comprises the amino acid sequence encoded by a human coding sequence selected from the group consisting of, SEQ ID NOS: 6, 12, 18, 24, 30 and 33 shown in Tables 1-6.

19. The polypeptide of claim 16, wherein said polypeptide comprises the amino acid sequence of an epitope of the amino acid sequence of a CA polypeptide selected from the group consisting of SEQ ID NOS: 6, 12, 18, 24, 30 and 33 shown in Tables 1-6.

20. The polypeptide of claim 16, wherein said polypeptide or fragment thereof is attached to a solid support.

21. An isolated antibody or antigen binding fragment thereof, that binds to a polypeptide according to anyone of claims 16-20.

22. The isolated antibody or antigen binding fragment thereof according the claim 21, wherein said antibody or fragment thereof is attached to a solid support.

23. The isolated antibody or antigen binding fragment thereof according the claim 21, wherein said antibody is a monoclonal antibody.

24. The isolated antibody or antigen binding fragment thereof according the claim 21, wherein said antibody is a polyclonal antibody.

25. The isolated antibody or antigen binding fragment thereof according the claim 21, wherein said antibody or fragment thereof further comprises a detectable label.

26. An isolated antibody that binds to a polypeptide, or antigen binding fragment thereof, according to any of claims 16-20, prepared by a method comprising the following steps of: (i) immunizing a host animal with a composition comprising said polypeptide, or antigen binding fragment thereof, and ii) collecting cells from said host expressing antibodies against the antigen or antigen binding fragment thereof.

27. A kit for diagnosing the presence of cancer in a test sample, said kit comprising at least one polynucleotide that selectively hybridizes to a CA polynucleotide sequence selected from the group consisting of the polynucleotide sequences of SEQ ID NOS: 4, 10, 16, 22, 28 and 31 shown in Tables 1-6, a fragment thereof, or their complement.

28. A kit for diagnosing the presence of cancer in a test sample, said kit comprising at least one polynucleotide that selectively hybridizes to the sequence of a polynucleotide sequence selected from the group consisting of the polynucleotide sequences of SEQ ID NOS: 5, 11, 17, 23, 29 and 32 shown in Tables 1-6, a fragment thereof, or their complement.

29. An electronic library comprising a polynucleotide, or fragment thereof, comprising a CA polynucleotide sequence selected from the group consisting of the polynucleotide sequences of SEQ ID NOS: 4, 10, 16, 22, 28 and 31 shown in Tables 1-6.

30. An electronic library comprising a polynucleotide, or fragment thereof, comprising a CA polynucleotide sequence selected from the group consisting of the polynucleotide sequences of SEQ ID NOS: 5, 11, 17, 23, 29 and 32 shown in Tables 1-6.

31. An electronic library comprising a polypeptide, or fragment thereof, comprising a CA polypeptide encoded by a polynucleotide of a sequence selected from the group consisting of the polynucleotide sequences of SEQ ID NOS: 6, 12, 18, 24, 30 and 33 shown in Tables 1-6.

32. A method for screening for anticancer activity in a potential drug, the method comprising:
(a) providing a cell that expresses a cancer associated (CA) gene encoded by a nucleic acid sequence selected from the group consisting of the sequences of SEQ ID NOS: 4, 10, 16, 22, 28 and 31 shown in Tables 1-6 or fragment thereof;
(b) contacting a tissue sample derived from a cancer cell with an anticancer drug candidate; and
(c) monitoring an effect of the anticancer drug candidate on an expression of the CA gene in the tissue sample.

33. The method of screening for anticancer activity according to claim 32, wherein the CA gene comprises at least one nucleic acid sequence selected from the group consisting of the sequences of SEQ ID NOS: 5, 11, 17, 23, 29 and 32 shown in Tables 1-6.

34. The method of screening for anticancer activity according to claim 32, further comprising:
(d) comparing the level of expression of the in the absence of said drug candidate to the level of expression in the presence of the drug candidate.

35. The method of screening for anticancer activity according to claim 33, wherein the drug candidate modulates the activity of a CAP sequence selected from the group consisting of SNL, FOSB, CCND1, MYC, NFKB1, and PVT1.

36. A method for detecting cancer associated with expression of a polypeptide in a test cell sample, comprising the steps of:
(i) detecting a level of expression of at least one polypeptide having an amino acid sequence encoded by a human coding sequence selected from the group consisting of of SEQ ID NOS: 6, 12, 18, 24, 30 and 33 shown in Tables 1-6, or a fragment thereof; and
(ii) comparing the level of expression of the polypeptide in the test sample with a level of expression of polypeptide in a normal cell sample, wherein an altered level of expression of the polypeptide in the test cell sample relative to the level of polypeptide expression in the normal cell sample is indicative of the presence of cancer in the test cell sample.

37. A method for detecting cancer associated with expression of a polypeptide in a test cell sample, comprising the steps of:
(i) detecting a level of activity of at least one polypeptide having an amino acid sequence encoded by a human coding sequence selected from the group consisting of of SEQ ID NOS: 6, 12, 18, 24, 30 and 33 shown in Tables 1-6, or a fragment thereof, wherein said activity corresponds to at least one activity for the polypeptide listed in Table 130; and
(ii) comparing the level of activity of the polypeptide in the test sample with a level of activity of polypeptide in a normal cell sample, wherein an altered level of activity of the polypeptide in the test cell sample relative to the level of polypeptide activity in the normal cell sample is indicative of the presence of cancer in the test cell sample.

38. A method for detecting cancer associated with the presence of an antibody in a test serum sample, comprising the steps of:
(i) detecting a level of an antibody against an antigenic polypeptide having an amino acid sequence encoded by a human coding sequence selected from the group consisting of of SEQ ID NOS: 6, 12, 18, 24, 30 and 33 shown in Tables 1-6, or antigenic fragment thereof; and
(ii) comparing said level of said antibody in the test sample with a level of said antibody in the control sample, wherein an altered level of antibody in said test sample relative to the level of antibody in the control sample is indicative of the presence of cancer in the test serum sample.

39. A method for screening for a bioactive agent capable of modulating the activity of a CA protein (CAP), wherein said CAP is encoded by a nucleic acid comprising a nucleic acid sequence selected from the group consisting of the polynucleotide sequences of SEQ ID NOS: 5, 11, 17, 23, 29 and 32 shown in Tables 1-6, said method comprising:
a) combining said CAP and a candidate bioactive agent; and
b) determining the effect of the candidate agent on the bioactivity of said CAP.

40. The method of screening for the bioactive agent according to claim 39, wherein the bioactive agent affects the expression of the CA protein (CAP).

41. The method of screening for the bioactive agent according to claim 39, wherein the bioactive agent affects the activity of the CA protein (CAP), wherein the CAP is selected from the group consisting of SNL, FOSB, CCND1, MYC, NFKB1, and PVT1.

42. A method for diagnosing cancer comprising:
a) determining the expression of one or more genes comprising a nucleic acid sequence selected from the group consisting of the human sequences outlined in Tables 1-6, in a first tissue type of a first individual; and
b) comparing said expression of said gene(s) from a second normal tissue type from said first individual or a second unaffected individual;
wherein a difference in said expression indicates that the first individual has cancer.

43. The method for diagnosing cancers according to claim 42, wherein the difference in said expression indicates that the first individual has a propensity towards cancer.

44. The method for diagnosing cancers according to claim 42, wherein the gene comprises SNL1 sequences corresponding to SEQ ID NOS: 4, 5 and 6 and the tissue is breast cancer tissue.

45. The method for diagnosing cancers according to claim 42, wherein the gene comprises FOSB sequences corresponding to SEQ ID NOS: 10-12 and the tissue is selected from the group consisting of colon cancer, lung cancer, pancreatic cancer, ovarian cancer, stomach cancer, breast cancer and prostate cancer tissue.

46. The method for diagnosing cancers according to claim 42, wherein the gene comprises MYC sequences corresponding to SEQ ID NOS: 22-24 and the tissue is breast cancer tissue.

47. The method for diagnosing cancers according to claim 42, wherein the gene comprises an CCND1 sequences corresponding to SEQ ID NOS: 16-18 and the tissue is selected from the group consisting of colon cancer (sigmoid), colon cancer (transverse), lung cancer, ovarian cancer, and breast cancer tissue.

48. The method for diagnosing cancers according to claim 42, wherein the gene comprises an NFKB1 sequences corresponding to SEQ ID NOS: 28-30 and the tissue is selected from the group consisting of lung cancer, skin cancer, and breast cancer tissue.

49. The method for diagnosing cancers according to claim 42, wherein the gene comprises PVT1 sequences corresponding to SEQ ID NOS: 31-33 and the tissue is breast cancer tissue.

50. The method for diagnosing cancers according to claim 42, wherein the gene expression in the cancer tissue is up-regulated relative to the gene expression in the normal tissue.

51. The method for diagnosing cancers according to claim 50, wherein the difference in said expression indicates that the first individual has a propensity towards cancer.

52. A method for diagnosing cancer or a propensity towards cancer comprising determining the amplification of one or more genes comprising a DNA sequence selected from the group consisting of the human sequences outlined in Tables 1-6, in a first tissue type of a first individual relative to a second normal tissue type from said first individual or a second unaffected individual, wherein an amplification of the DNA indicates that the first individual has cancer or a propensity towards cancer.

53. The method for diagnosing cancer or a propensity towards cancer according to claim 52, wherein the gene comprises MYC and the DNA sequence is SEQ ID NO: 22, or a fragment thereof.

54. The method for diagnosing cancer or a propensity towards cancer according to claim 52, wherein the gene comprises PVT1 and the DNA sequence is SEQ ID NO: 31, or a fragment thereof.

55. A method for treating cancers comprising administering to a patient an inhibitor of a CA protein (CAP), wherein said CAP is encoded by a nucleic acid comprising a human nucleic acid sequence selected from the group consisting of the sequences outlined in Tables 1-6.

56. The method for treating cancers according to claim 55, wherein the inhibitor of a CA protein (CAP) binds to the CA protein.

57. The method for treating cancers according to claim 55, wherein the inhibitor of a CA protein (CAP) modulates the activity of a CAP sequence selected from the group consisting of SNL, FOSB, CCND1, MYC, NFKB1, and PVT1.
